SEQUENCE LISTING (416 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - protein, 19 aa, synthetic construct
  SITE 16: note = X is 1,13-diamino-4,7,10-trioxatridecan-succinamic acid (1-amino-15-oxo-4,7,10-trioxa-14-azaoctadecan-18-oic acid)
  SITE 17: note = X is 1,13-diamino-4,7,10-trioxatridecan-succinamic acid (1-amino-15-oxo-4,7,10-trioxa-14-azaoctadecan-18-oic acid)
  SITE 18: note = X is 1,13-diamino-4,7,10-trioxatridecan-succinamic acid (1-amino-15-oxo-4,7,10-trioxa-14-azaoctadecan-18-oic acid)
  SITE 19: note = X is L-lysine with D-biotin attached to side chain
  MOD_RES 18: note = AMIDATION
  DISULFID 5..10: note = Cys Cys connected through disulfide bond
MTAFCKRANCLTDGSXXXX

SEQ ID NO: 3 - protein, 15 aa, synthetic construct
  MOD_RES 14: note = AMIDATION
  DISULFID 5..10: note = Cys Cys connected through disulfide bond
MTAFCKRANCLTDGS

SEQ ID NO: 4 - protein, 15 aa, synthetic construct
  SITE 1: note = X is acetyl
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTAFCKRANCLTDXX

SEQ ID NO: 5 - protein, 14 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  MOD_RES 13: note = AMIDATION
  DISULFID 6..11: note = Cys Cys connected through disulfide bond
XXTAFCKRANCLTD

SEQ ID NO: 6 - protein, 15 aa, synthetic construct
  SITE 1: note = X is n-hexanoic acid
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTAFCKRANCLTDXX

SEQ ID NO: 7 - protein, 13 aa, synthetic construct
  SITE 1: note = X is acetyl
  SITE 12: note = X is 1,13-diamino-4,7,10-trioxatridecan-succinamic acid (1-amino-15-oxo-4,7,10-trioxa-14-azaoctadecan-18-oic acid)
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 3..8: note = Cys Cys connected through disulfide bond
XFCKRANCLTDXX

SEQ ID NO: 8 - protein, 14 aa, synthetic construct
  SITE 1: note = X is acetyl
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 4..9: note = Cys Cys connected through disulfide bond
XAFCKRANCLTDXX

SEQ ID NO: 9 - protein, 12 aa, synthetic construct
  SITE 1: note = X is acetyl
  SITE 11: note = X is 1,13-diamino-4,7,10-trioxatridecan-succinamic acid (1-amino-15-oxo-4,7,10-trioxa-14-azaoctadecan-18-oic acid)
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 2..7: note = Cys Cys connected through disulfide bond
XCKRANCLTDXX

SEQ ID NO: 10 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  MOD_RES 12: note = AMIDATION
  DISULFID 6..11: note = Cys Cys connected through disulfide bond
XXTAFCKRANCLT

SEQ ID NO: 11 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  MOD_RES 11: note = AMIDATION
  DISULFID 6..11: note = Cys Cys connected through disulfide bond
XXTAFCKRANCL

SEQ ID NO: 12 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  MOD_RES 10: note = AMIDATION
  DISULFID 6..11: note = Cys Cys connected through disulfide bond
XXTAFCKRANC

SEQ ID NO: 13 - protein, 14 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 12: note = X is (S)-2-aminohexanoic acid
  MOD_RES 13: note = AMIDATION
  DISULFID 6..11: note = Cys Cys connected through disulfide bond
XXTAFCKRANCXTD

SEQ ID NO: 14 - protein, 15 aa, synthetic construct
  SITE 1: note = X is acetyl
  SITE 11: note = X is (S)-2-aminohexanoic acid
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTAFCKRANCXTDXX

SEQ ID NO: 15 - protein, 15 aa, synthetic construct
  SITE 1: note = X is acetyl
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTAFCKRANCXTDXX

SEQ ID NO: 16 - protein, 14 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 12: note = X is tert leucine
  MOD_RES 13: note = AMIDATION
  DISULFID 6..11: note = Cys Cys connected through disulfide bond
XXTAFCKRANCXTD

SEQ ID NO: 17 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRANCXTD

SEQ ID NO: 18 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRENCXTD

SEQ ID NO: 19 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTPFCKRANCXTD

SEQ ID NO: 20 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCPRANCXTD

SEQ ID NO: 21 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKPANCXTD

SEQ ID NO: 22 - protein, 13 aa, synthetic construct
  SITE 1: note = X is acetyl
  SITE 8: note = X is D-alanine
  SITE 11: note = X is (S)-2-aminohexanoic acid
  MOD_RES 12: note = AMIDATION
  DISULFID 5..10: note = Cys Cys connected through disulfide bond
XTAFCKRXNCXTD

SEQ ID NO: 23 - protein, 13 aa, synthetic construct
  SITE 1: note = X is acetyl
  SITE 8: note = X is 2-amino-2-methylpropanoic acid
  SITE 11: note = X is (S)-2-aminohexanoic acid
  MOD_RES 12: note = AMIDATION
  DISULFID 5..10: note = Cys Cys connected through disulfide bond
XTAFCKRXNCXTD

SEQ ID NO: 24 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRPNCXTD

SEQ ID NO: 25 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRAPCXTD

SEQ ID NO: 26 - protein, 13 aa, synthetic construct
  SITE 1: note = X is acetyl
  SITE 11: note = X is (S)-2-aminohexanoic acid
  SITE 12: note = X is D-threonine
  MOD_RES 12: note = AMIDATION
  DISULFID 5..10: note = Cys Cys connected through disulfide bond
XTAFCKRANCXXD

SEQ ID NO: 27 - protein, 13 aa, synthetic construct
  SITE 1: note = X is acetyl
  SITE 11: note = X is (S)-2-aminohexanoic acid
  SITE 13: note = X is D-aspartic acid
  MOD_RES 12: note = AMIDATION
  DISULFID 5..10: note = Cys Cys connected through disulfide bond
XTAFCKRANCXTX

SEQ ID NO: 28 - protein, 13 aa, synthetic construct
  SITE 1: note = X is acetyl
  SITE 2: note = X is D-threonine
  SITE 11: note = X is (S)-2-aminohexanoic acid
  MOD_RES 12: note = AMIDATION
  DISULFID 5..10: note = Cys Cys connected through disulfide bond
XXAFCKRANCXTD

SEQ ID NO: 29 - protein, 13 aa, synthetic construct
  SITE 1: note = X is acetyl
  SITE 3: note = X is D-alanine
  SITE 11: note = X is (S)-2-aminohexanoic acid
  MOD_RES 12: note = AMIDATION
  DISULFID 5..10: note = Cys Cys connected through disulfide bond
XTXFCKRANCXTD

SEQ ID NO: 30 - protein, 13 aa, synthetic construct
  SITE 1: note = X is acetyl
  SITE 4: note = X is D-phenyl alanine
  SITE 11: note = X is (S)-2-aminohexanoic acid
  MOD_RES 12: note = AMIDATION
  DISULFID 5..10: note = Cys Cys connected through disulfide bond
XTAXCKRANCXTD

SEQ ID NO: 31 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XAAFCKRANCXTD

SEQ ID NO: 32 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCARANCXTD

SEQ ID NO: 33 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKAANCXTD

SEQ ID NO: 34 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRAACXTD

SEQ ID NO: 35 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRANCXAD

SEQ ID NO: 36 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAFCKRANCXTA

SEQ ID NO: 37 - protein, 13 aa, synthetic construct
  SITE 1: note = X is acetyl
  SITE 11: note = X is (S)-2-aminohexanoic acid
  SITE 13: note = X is 4-aminobutanoic acid
  DISULFID 5..10: note = Cys Cys connected through disulfide bond
XTAFCKRANCXTX

SEQ ID NO: 38 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XSAFCKRANCXTD

SEQ ID NO: 39 - protein, 13 aa, synthetic construct
  SITE 1: note = X is acetyl
  SITE 3: note = X is 2-amino-2-methylpropanoic acid
  SITE 11: note = X is (S)-2-aminohexanoic acid
  MOD_RES 12: note = AMIDATION
  DISULFID 5..10: note = Cys Cys connected through disulfide bond
XTXFCKRANCXTD

SEQ ID NO: 40 - protein, 13 aa, synthetic construct
  SITE 1: note = X is acetyl
  SITE 11: note = X is (S)-2-aminohexanoic acid
  MOD_RES 12: note = AMIDATION
  DISULFID 5..10: note = Cys Cys connected through disulfide bond
XTAACKRANCXTD

SEQ ID NO: 41 - protein, 15 aa, synthetic construct
  SITE 1: note = X is n-hexanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTAFCKRANCXTDXX

SEQ ID NO: 42 - protein, 13 aa, synthetic construct
  SITE 1: note = X is acetyl
  SITE 3: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKRANCXTD

SEQ ID NO: 43 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 44 - protein, 13 aa, synthetic construct
  SITE 1: note = X is acetyl
  SITE 3: note = X is 2-amino-2-methylpropanoic acid
  SITE 8: note = X is hydroxyproline ((2S,4R)-4-hydroxypyrrolidine-2-carboxylic acid)
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 45 - protein, 13 aa, synthetic construct
  SITE 1: note = X is acetyl
  SITE 3: note = X is 2-amino-2-methylpropanoic acid
  SITE 8: note = X is (2S,3aS,7aS)-octahydro-1H-indole-2-carboxylic acid
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 46 - protein, 13 aa, synthetic construct
  SITE 1: note = X is acetyl
  SITE 3: note = X is 2-amino-2-methylpropanoic acid
  SITE 8: note = X is (R)-5,5-dimethylthiazolidine-4-carboxylic acid
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 47 - protein, 13 aa, synthetic construct
  SITE 1: note = X is acetyl
  SITE 3: note = X is 2-amino-2-methylpropanoic acid
  SITE 8: note = X is (2S,4R)-4-fluoropyrrolidine-2-carboxylic acid
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 48 - protein, 13 aa, synthetic construct
  SITE 1: note = X is acetyl
  SITE 3: note = X is 2-amino-2-methylpropanoic acid
  SITE 6: note = X is (2S,4R)-4-amino-pyrrolidine-2-carboxylic acid
  SITE 11: note = X is tert leucine
  MOD_RES 12: note = AMIDATION
  DISULFID 5..10: note = Cys Cys connected through disulfide bond
XTXFCXRANCXTD

SEQ ID NO: 49 - protein, 13 aa, synthetic construct
  SITE 1: note = X is acetyl
  SITE 3: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCERANCXTD

SEQ ID NO: 50 - protein, 13 aa, synthetic construct
  SITE 1: note = X is acetyl
  SITE 3: note = X is 2-amino-2-methylpropanoic acid
  SITE 6: note = X is (S)-2-amino-3-(4-aminophenyl)propanoic acid
  SITE 11: note = X is tert leucine
  MOD_RES 12: note = AMIDATION
  DISULFID 5..10: note = Cys Cys connected through disulfide bond
XTXFCXRANCXTD

SEQ ID NO: 51 - protein, 13 aa, synthetic construct
  SITE 1: note = X is acetyl
  SITE 3: note = X is 2-amino-2-methylpropanoic acid
  SITE 7: note = X is citrulline
  SITE 11: note = X is tert leucine
  MOD_RES 12: note = AMIDATION
  DISULFID 5..10: note = Cys Cys connected through disulfide bond
XTXFCKXANCXTD

SEQ ID NO: 52 - protein, 13 aa, synthetic construct
  SITE 1: note = X is acetyl
  SITE 3: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKQANCXTD

SEQ ID NO: 53 - protein, 13 aa, synthetic construct
  SITE 1: note = X is acetyl
  SITE 3: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCQRANCXTD

SEQ ID NO: 54 - protein, 13 aa, synthetic construct
  SITE 1: note = X is acetyl
  SITE 3: note = X is 2-amino-2-methylpropanoic acid
  SITE 6: note = X is D-lysine
  SITE 11: note = X is tert leucine
  MOD_RES 12: note = AMIDATION
  DISULFID 5..10: note = Cys Cys connected through disulfide bond
XTXFCXRANCXTD

SEQ ID NO: 55 - protein, 13 aa, synthetic construct
  SITE 1: note = X is acetyl
  SITE 3: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKEANCXTD

SEQ ID NO: 56 - protein, 13 aa, synthetic construct
  SITE 1: note = X is acetyl
  SITE 3: note = X is 2-amino-2-methylpropanoic acid
  SITE 7: note = X is D-arginine
  SITE 11: note = X is tert leucine
  MOD_RES 12: note = AMIDATION
  DISULFID 5..10: note = Cys Cys connected through disulfide bond
XTXFCKXANCXTD

SEQ ID NO: 57 - protein, 13 aa, synthetic construct
  SITE 1: note = X is acetyl
  SITE 3: note = X is 2-amino-2-methylpropanoic acid
  SITE 8: note = X is (S)-4,4-difluoropyrrolidine-2-carboxylic acid
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 58 - protein, 13 aa, synthetic construct
  SITE 1: note = X is acetyl
  SITE 3: note = X is 2-amino-2-methylpropanoic acid
  SITE 5: note = X is S-methyl cysteine
  SITE 11: note = X is tert leucine
  MOD_RES 12: note = AMIDATION
  CROSSLNK 5..10: note = Smc Cys connected through a methylene bond (S-CH2-S)
XTXFXKRANCXTD

SEQ ID NO: 59 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCQRXNCXTD

SEQ ID NO: 60 - protein, 13 aa, synthetic construct
  SITE 1: note = X is acetyl
  SITE 3: note = X is 2-amino-2-methylpropanoic acid
  SITE 7: note = X is citrulline
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKXXNCXTD

SEQ ID NO: 61 - protein, 12 aa, synthetic construct
  SITE 1: note = X is acetyl
  SITE 3: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRANCXT

SEQ ID NO: 62 - protein, 12 aa, synthetic construct
  SITE 1: note = X is acetyl
  SITE 2: note = X is 2-amino-2-methylpropanoic acid
  SITE 10: note = X is tert leucine
  MOD_RES 11: note = AMIDATION
  DISULFID 4..9: note = Cys Cys connected through disulfide bond
XXFCKRANCXTD

SEQ ID NO: 63 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKQXNCXTD

SEQ ID NO: 64 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXFCKEXNCXTD

SEQ ID NO: 65 - protein, 12 aa, synthetic construct
  SITE 1: note = X is acetyl
  SITE 2: note = X is 2-amino-2-methylpropanoic acid
  SITE 7: note = X is 2-amino-2-methylpropanoic acid
  SITE 10: note = X is tert leucine
  MOD_RES 11: note = AMIDATION
  DISULFID 4..9: note = Cys Cys connected through disulfide bond
XXFCKRXNCXTD

SEQ ID NO: 66 - protein, 15 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTXFCKRXNCXTDXX

SEQ ID NO: 67 - protein, 14 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 9: note = X is 2-amino-2-methylpropanoic acid
  SITE 12: note = X is tert leucine
  MOD_RES 13: note = AMIDATION
  DISULFID 6..11: note = Cys Cys connected through disulfide bond
XXTXFCKRXNCXTD

SEQ ID NO: 68 - protein, 14 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 13: note = AMIDATION
  DISULFID 6..11: note = Cys Cys connected through disulfide bond
XXTXFCKRANCXTD

SEQ ID NO: 69 - protein, 13 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 70 - protein, 15 aa, synthetic construct
  SITE 1: note = X is n-hexanoic acid
  SITE 3: note = X is 2-amino-2-methylpropanoic acid
  SITE 8: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTXFCKRXNCXTDXX

SEQ ID NO: 71 - protein, 15 aa, synthetic construct
  SITE 1: note = X is n-hexanoic acid
  SITE 3: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTXFCKRANCXTDXX

SEQ ID NO: 72 - protein, 15 aa, synthetic construct
  SITE 1: note = X is acetyl
  SITE 3: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 14: note = AMIDATION
  DISULFID 5..10: note = Cys Cys connected through disulfide bond
XTXFCKRANCXTDXX

SEQ ID NO: 73 - protein, 13 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Ttds linker (1-amino-15-oxo-4,7,10-trioxa-14-azaoctadecan-18-oic acid) interspersed
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 74 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 75 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKQXNCXT

SEQ ID NO: 76 - protein, 14 aa, synthetic construct
  SITE 1: note = X is n-hexanoic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 77 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain
  SITE 11: note = X is tert leucine
  MOD_RES 11: note = AMIDATION
  DISULFID 5..10: note = Cys Cys connected through disulfide bond
XTXFCKRXNCXT

SEQ ID NO: 78 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKXXNCXT

SEQ ID NO: 79 - protein, 14 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 13: note = AMIDATION
  DISULFID 6..11: note = Cys Cys connected through disulfide bond
XXTXFCKRANCXTD

SEQ ID NO: 80 - protein, 13 aa, synthetic construct
  SITE 1: note = X is acetyl
  SITE 3: note = X is 2-amino-2-methylpropanoic acid
  SITE 8: note = X is lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 81 - protein, 15 aa, synthetic construct
  SITE 1: note = X is n-hexanoic acid
  SITE 3: note = X is 2-amino-2-methylpropanoic acid
  SITE 8: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 14: note = AMIDATION
  DISULFID 5..10: note = Cys Cys connected through disulfide bond
XTXFCKRXNCXTDXX

SEQ ID NO: 82 - protein, 13 aa, synthetic construct
  SITE 1: note = X is acetyl
  SITE 3: note = X is 2-amino-2-methylpropanoic acid
  SITE 8: note = X is lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid) attached to side chain with Ttds linker (1-amino-15-oxo-4,7,10-trioxa-14-azaoctadecan-18-oic acid) interspersed
  SITE 11: note = X is tert leucine
  MOD_RES 12: note = AMIDATION
  DISULFID 5..10: note = Cys Cys connected through disulfide bond
XTXFCKRXNCXTD

SEQ ID NO: 83 - protein, 14 aa, synthetic construct
  SITE 1: note = X is acetyl
  SITE 3: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRANCXTXX

SEQ ID NO: 84 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Ttds linker (1-amino-15-oxo-4,7,10-trioxa-14-azaoctadecan-18-oic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 85 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain
  SITE 11: note = X is tert leucine
  MOD_RES 11: note = AMIDATION
  DISULFID 5..10: note = Cys Cys connected through disulfide bond
XTXFCKQXNCXT

SEQ ID NO: 86 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Ttds linker (1-amino-15-oxo-4,7,10-trioxa-14-azaoctadecan-18-oic acid) interspersed
  SITE 11: note = X is tert leucine
  MOD_RES 11: note = AMIDATION
  DISULFID 5..10: note = Cys Cys connected through disulfide bond
XTXFCKQXNCXT

SEQ ID NO: 87 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRANCXT

SEQ ID NO: 88 - protein, 14 aa, synthetic construct
  SITE 1: note = X is n-hexanoic acid
  SITE 3: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRANCXTXX

SEQ ID NO: 89 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 90 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKQXNCXTXX

SEQ ID NO: 91 - protein, 14 aa, synthetic construct
  SITE 1: note = X is n-hexanoic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKQXNCXTXX

SEQ ID NO: 92 - protein, 13 aa, synthetic construct
  SITE 1: note = X is acetyl
  SITE 3: note = X is 2-amino-2-methylpropanoic acid
  SITE 4: note = X is (S)-2-amino-3-(naphthalen-1-yl)propanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXXCKRXNCXTD

SEQ ID NO: 93 - protein, 13 aa, synthetic construct
  SITE 1: note = X is acetyl
  SITE 3: note = X is 2-amino-2-methylpropanoic acid
  SITE 4: note = X is (S)-2-amino-3-(naphthalen-2-yl)propanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXXCKRXNCXTD

SEQ ID NO: 94 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXWCKRXNCXTD

SEQ ID NO: 95 - protein, 13 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXXCKRXNCXTD

SEQ ID NO: 96 - protein, 13 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXXCKRXNCXTD

SEQ ID NO: 97 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXYCKRXNCXTD

SEQ ID NO: 98 - protein, 13 aa, synthetic construct
  SITE 1: note = X is acetyl
  SITE 3: note = X is 2-amino-2-methylpropanoic acid
  SITE 4: note = X is (S)-2-amino-3-(2-chlorophenyl)propanoic acid
  SITE 8: note = X is 2-amino-2-methylpropanoic acid
  SITE 11: note = X is tert leucine
  MOD_RES 12: note = AMIDATION
  DISULFID 5..10: note = Cys Cys connected through disulfide bond
XTXXCKRXNCXTD

SEQ ID NO: 99 - protein, 9 aa, synthetic construct
  SITE 1: note = X is acetyl
  MOD_RES 8: note = AMIDATION
  DISULFID 3..8: note = Cys Cys connected through disulfide bond
XNCKRWACK

SEQ ID NO: 100 - protein, 9 aa, synthetic construct
  SITE 1: note = X is acetyl
  MOD_RES 8: note = AMIDATION
  DISULFID 3..8: note = Cys Cys connected through disulfide bond
XRCKGVMCK

SEQ ID NO: 101 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 4-(aminomethyl)benzoic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 102 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 103 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(2-(2-aminoethoxy)ethoxy)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 104 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 105 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 6-aminohexanoic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 106 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 3-aminopropanoic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 107 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Cmp linker (2-(piperidin-4-yl)acetic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 108 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(4-aminopiperidin-1-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 109 - protein, 13 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 5..10: note = Cys Cys connected through disulfide bond
XTXFCKRXNCXTX

SEQ ID NO: 110 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(4-aminopiperidin-1-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 111 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 112 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(2-(2-aminoethoxy)ethoxy)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 113 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 114 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Pamb linker (4-(aminomethyl)benzoic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 115 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with APAc linker (2-(4-(amino)piperidin-1-yl)acetic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 116 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 117 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Ahx linker (6-aminohexanoic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 118 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is pentane-1,5-diamine
  SITE 14: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 5..10: note = Cys Cys connected through disulfide bond
XTXFCKRXNCXTXX

SEQ ID NO: 119 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 4-aminobenzoic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 120 - protein, 15 aa, synthetic construct
  SITE 1: note = X is 2-(6-amino-3-imino-4,5-disulfo-3H-xanthen-9-yl )-5-((3-(1-(5-carboxypentyl)-1,9-dihydro-8H-dibenzo[b,f][1 ,2,3]triazolo[4,5-d]azocin-8-yl)-3-oxopropyl)carbamoyl)ben zoic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 3: note = X is 1,13-diamino-4,7,10-trioxatridecan-succinamic acid (1-amino-15-oxo-4,7,10-trioxa-14-azaoctadecan-18-oic acid)
  SITE 5: note = X is 2-amino-2-methylpropanoic acid
  SITE 10: note = X is 2-amino-2-methylpropanoic acid
  SITE 13: note = X is tert leucine
  MOD_RES 14: note = AMIDATION
  DISULFID 7..12: note = Cys Cys connected through disulfide bond
XXXTXFCKRXNCXTD

SEQ ID NO: 121 - protein, 16 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is 1,13-diamino-4,7,10-trioxatridecan-succinamic acid (1-amino-15-oxo-4,7,10-trioxa-14-azaoctadecan-18-oic acid)
  SITE 16: note = X is 2-(6-amino-3-imino-4,5-disulfo-3H-xanthen-9-yl )-5-((3-(1-((R)-5-amino-6-(((S)-4-amino-1-(((S)-1-carboxy- 2-(1H-imidazol-4-yl)ethyl)amino)-1,4-dioxobutan-2-yl)amino )-6-oxohexyl)-1,9-dihydro-8H-dibenzo[b,f][1,2,3]triazolo[4 ,5-d]azocin-8-yl)-3-oxopropyl)carbamoyl)benzoic acid
  MOD_RES 15: note = AMIDATION
  DISULFID 5..10: note = Cys Cys connected through disulfide bond
XTXFCKRXNCXTDXXX

SEQ ID NO: 122 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-aminobenzoic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 123 - protein, 12 aa, synthetic construct
  SITE 1: note = X is acetyl
  SITE 3: note = X is 2-amino-2-methylpropanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with Pab linker (4-amino benzoic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 124 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 125 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-chlorophenyl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 126 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 127 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 128 - protein, 13 aa, synthetic construct
  SITE 1: note = X is 2-[1,4,7,10-Tetraazacyclododecane-4,7,10-trisa cetate]-pentanedioic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 129 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXXCKRXNCXT

SEQ ID NO: 130 - protein, 12 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXXCKRXNCXT

SEQ ID NO: 131 - protein, 14 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 132 - protein, 14 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 133 - protein, 14 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKRXNCXTXX

SEQ ID NO: 134 - protein, 12 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 7: note = X is citrulline
  SITE 8: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXXCKXXNCXT

SEQ ID NO: 135 - protein, 14 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKRXNCXTXX

SEQ ID NO: 136 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTAGA (2-[1,4,7,10-Tetraazacycl ododecane-4,7,10-trisacetate]-pentanedioic acid) attached to side chain
  MOD_RES 13: note = AMIDATION
  DISULFID 5..10: note = Cys Cys connected through disulfide bond
XTXFCKRXNCXTXX

SEQ ID NO: 137 - protein, 12 aa, synthetic construct
  SITE 1: note = X is acetyl
  SITE 3: note = X is 2-amino-2-methylpropanoic acid
  SITE 4: note = X is (S)-2-amino-3-(3-chlorophenyl)propanoic acid
  SITE 8: note = X is lysine with DOTAGA (2-[1,4,7,10-Tetraazacyclod odecane-4,7,10-trisacetate]-pentanedioic acid) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXXCKRXNCXT

SEQ ID NO: 138 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKRXNCXTXX

SEQ ID NO: 139 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(3-chlorophenyl)propanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKRXNCXTXX

SEQ ID NO: 140 - protein, 14 aa, synthetic construct
  SITE 1: note = X is butylcarbamic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 141 - protein, 13 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 142 - protein, 12 aa, synthetic construct
  SITE 1: note = X is acetyl
  SITE 3: note = X is 2-amino-2-methylpropanoic acid
  SITE 8: note = X is lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain with O2Oc linker (2-(2-(2-aminoethoxy)ethoxy)acetic acid) interspersed
  SITE 11: note = X is tert leucine
  MOD_RES 11: note = AMIDATION
  DISULFID 5..10: note = Cys Cys connected through disulfide bond
XTXFCKRXNCXT

SEQ ID NO: 143 - protein, 14 aa, synthetic construct
  SITE 1: note = X is butylcarbamic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 13: note = AMIDATION
  DISULFID 5..10: note = Cys Cys connected through disulfide bond
XTXFCKRXNCXTXX

SEQ ID NO: 144 - protein, 14 aa, synthetic construct
  SITE 1: note = X is butylcarbamic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with LuDOTA (Lu(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 13: note = AMIDATION
  DISULFID 5..10: note = Cys Cys connected through disulfide bond
XTXFCKRXNCXTXX

SEQ ID NO: 145 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 146 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 147 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-3-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 148 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-4-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 149 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(quinolin-3-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 150 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3,4-dichlorophenyl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 151 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(5-chloro-1H-indol-3-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 152 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(5-bromo-1H-indol-3-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 153 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(6-chloro-1H-indol-3-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 154 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXLXFCKRXNCXT

SEQ ID NO: 155 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXIXFCKRXNCXT

SEQ ID NO: 156 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(4-chlorophenyl)propanoic acid
  SITE 7: note = X is (S)-2-amino-5-(pyridin-2-ylamino)pentanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 157 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(3-chlorophenyl)propanoic acid
  SITE 7: note = X is (S)-2-amino-5-(pyridin-2-ylamino)pentanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 158 - protein, 14 aa, synthetic construct
  SITE 1: note = X is butylcarbamic acid
  SITE 3: note = X is 2-amino-2-methylpropanoic acid
  SITE 7: note = X is (S)-2-amino-5-(pyridin-2-ylamino)pentanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKXXNCXTXX

SEQ ID NO: 159 - protein, 15 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is D-leucine
  SITE 5: note = X is D-glutamic acid
  SITE 6: note = X is 2-amino-2-methylpropanoic acid
  SITE 11: note = X is 2-amino-2-methylpropanoic acid
  SITE 14: note = X is tert leucine
  MOD_RES 14: note = AMIDATION
  DISULFID 8..13: note = Cys Cys connected through disulfide bond
XXTXXXFCKRXNCXT

SEQ ID NO: 160 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 161 - protein, 14 aa, synthetic construct
  SITE 1: note = X is butylcarbamic acid
  SITE 3: note = X is 2-amino-2-methylpropanoic acid
  SITE 4: note = X is (S)-2-amino-3-(3-chlorophenyl)propanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXXCKXXNCXTXX

SEQ ID NO: 162 - protein, 14 aa, synthetic construct
  SITE 1: note = X is butylcarbamic acid
  SITE 3: note = X is 2-amino-2-methylpropanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKXXNCXTXX

SEQ ID NO: 163 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-2-yl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 164 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-(carboxymethyl)phenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 165 - protein, 14 aa, synthetic construct
  SITE 1: note = X is acetyl
  SITE 3: note = X is 2-amino-2-methylpropanoic acid
  SITE 7: note = X is citrulline
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKXXNCXTXX

SEQ ID NO: 166 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-sulfophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 167 - protein, 14 aa, synthetic construct
  SITE 1: note = X is 2-(piperidin-4-yl)acetic acid
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 13: note = AMIDATION
  DISULFID 5..10: note = Cys Cys connected through disulfide bond
XTXFCKRXNCXTXX

SEQ ID NO: 168 - protein, 13 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKXXNCXTX

SEQ ID NO: 169 - protein, 13 aa, synthetic construct
  SITE 1: note = X is n-hexanoic acid
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKXXNCXTX

SEQ ID NO: 170 - protein, 13 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKRXNCXTX

SEQ ID NO: 171 - protein, 13 aa, synthetic construct
  SITE 1: note = X is n-hexanoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKRXNCXTX

SEQ ID NO: 172 - protein, 13 aa, synthetic construct
  SITE 1: note = X is butylcarbamic acid
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKXXNCXTX

SEQ ID NO: 173 - protein, 13 aa, synthetic construct
  SITE 1: note = X is butylcarbamic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain with Cmp linker (2-(piperidin-4-yl)acetic acid) interspersed
  MOD_RES 12: note = AMIDATION
  DISULFID 5..10: note = Cys Cys connected through disulfide bond
XTXXCKRXNCXTX

SEQ ID NO: 174 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXRCXT

SEQ ID NO: 175 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXLCXT

SEQ ID NO: 176 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXWCXT

SEQ ID NO: 177 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXSCXT

SEQ ID NO: 178 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXECXT

SEQ ID NO: 179 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is D-leucine
  MOD_RES 11: note = AMIDATION
  DISULFID 5..10: note = Cys Cys connected through disulfide bond
XTXXCKXXNCXT

SEQ ID NO: 180 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is (S)-2-aminohexanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXNCXT

SEQ ID NO: 181 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 6: note = X is (S)-2-aminohexanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCXRXNCXT

SEQ ID NO: 182 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is citrulline
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXNCXT

SEQ ID NO: 183 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 184 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXF

SEQ ID NO: 185 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXECXT

SEQ ID NO: 186 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXDCXT

SEQ ID NO: 187 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXFCXT

SEQ ID NO: 188 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXGCXT

SEQ ID NO: 189 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is (S)-2-amino-5-guanidino-5-oxopentanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXNCXT

SEQ ID NO: 190 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 7: note = X is N-omega-acetyl-L-arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKXXNCXT

SEQ ID NO: 191 - protein, 11 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXXCKRXNCXT

SEQ ID NO: 192 - protein, 11 aa, synthetic construct
  SITE 1: note = X is acetyl
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 193 - protein, 11 aa, synthetic construct
  SITE 1: note = X is 3-hydroxy propionic acid
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 194 - protein, 11 aa, synthetic construct
  SITE 1: note = X is iso valeric acid (3-methylbutanoic acid)
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 195 - protein, 11 aa, synthetic construct
  SITE 1: note = X is succinic acid
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 196 - protein, 11 aa, synthetic construct
  SITE 1: note = X is 3-sufamoylpropanoic Acid
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 197 - protein, 12 aa, synthetic construct
  SITE 1: note = X is acetyl
  SITE 3: note = X is 2-amino-2-methylpropanoic acid
  SITE 8: note = X is (S)-2-methylpyrrolidine-2-carboxylic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 198 - protein, 12 aa, synthetic construct
  SITE 1: note = X is acetyl
  SITE 3: note = X is 2-amino-2-methylpropanoic acid
  SITE 8: note = X is 2-amino-2-ethylbutanoic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 199 - protein, 12 aa, synthetic construct
  SITE 1: note = X is acetyl
  SITE 3: note = X is 2-amino-2-methylpropanoic acid
  SITE 8: note = X is 4-amino-piperidine-4-carboxylic acid with DOTA (2,2 &apos;,2 &apos; &apos;,2 &apos; &apos; &apos;-(1,4,7, 10-Tetraazacyclododecane-1,4,7,10-tetrayl)tetraacetic acid) attached
  SITE 11: note = X is tert leucine
  MOD_RES 11: note = AMIDATION
  DISULFID 5..10: note = Cys Cys connected through disulfide bond
XTXFCKRXNCXT

SEQ ID NO: 200 - protein, 12 aa, synthetic construct
  SITE 1: note = X is acetyl
  SITE 3: note = X is 2-amino-2-ethylbutanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 201 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXW

SEQ ID NO: 202 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXLCXT

SEQ ID NO: 203 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXWCXT

SEQ ID NO: 204 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 9: note = X is D-asparagine
  SITE 11: note = X is tert leucine
  MOD_RES 11: note = AMIDATION
  DISULFID 5..10: note = Cys Cys connected through disulfide bond
XTXFCKRXXCXT

SEQ ID NO: 205 - protein, 11 aa, synthetic construct
  SITE 1: note = X is Hydantoin-5-acetic acid (2-(2,5-dioxoimidazolidin-4-yl)acetic acid)
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 206 - protein, 12 aa, synthetic construct
  SITE 1: note = X is acetyl
  SITE 3: note = X is (S)-2-methylpyrrolidine-2-carboxylic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 207 - protein, 12 aa, synthetic construct
  SITE 1: note = X is acetyl
  SITE 3: note = X is N-alpha methyl glycine/sarcosine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXT

SEQ ID NO: 208 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 209 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 6-aminohexanoic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 210 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(4-aminopiperidin-1-yl)acetic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 211 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-amino-2-methylpropanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXFCKRXNCXT

SEQ ID NO: 212 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperazin-1-yl)acetic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 213 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXSCXT

SEQ ID NO: 214 - protein, 12 aa, synthetic construct
  SITE 1: note = X is acetyl
  SITE 3: note = X is 2-amino-2-methylpropanoic acid
  SITE 5: note = X is D-cysteine
  SITE 8: note = X is 2-amino-2-methylpropanoic acid
  SITE 11: note = X is tert leucine
  MOD_RES 11: note = AMIDATION
  DISULFID 5..10: note = D-Cys Cys connected through disulfide bond
XTXFXKRXNCXT

SEQ ID NO: 215 - protein, 12 aa, synthetic construct
  SITE 1: note = X is acetyl
  SITE 3: note = X is 2-amino-2-methylpropanoic acid
  SITE 5: note = X is D-cysteine
  SITE 8: note = X is 2-amino-2-methylpropanoic acid
  SITE 10: note = X is D-cysteine
  SITE 11: note = X is tert leucine
  MOD_RES 11: note = AMIDATION
  DISULFID 5..10: note = D-Cys D-Cys connected through disulfide bond
XTXFXKRXNXXT

SEQ ID NO: 216 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 10: note = X is D-cysteine
  SITE 11: note = X is tert leucine
  MOD_RES 11: note = AMIDATION
  DISULFID 5..10: note = Cys D-Cys connected through disulfide bond
XTXFCKRXNXXT

SEQ ID NO: 217 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 3-aminopropanoic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 218 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 219 - protein, 12 aa, synthetic construct
  SITE 1: note = X is acetyl
  SITE 3: note = X is 2-amino-2-methylpropanoic acid
  SITE 8: note = X is N-alpha methyl glycine/sarcosine
  SITE 11: note = X is tert leucine
  MOD_RES 11: note = AMIDATION
  DISULFID 5..10: note = Cys Cys connected through disulfide bond
XTXFCKRXNCXT

SEQ ID NO: 220 - protein, 12 aa, synthetic construct
  SITE 1: note = X is acetyl
  SITE 3: note = X is 2-amino-2-methylpropanoic acid
  SITE 6: note = X is N-alpha methyl lysine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCXRXNCXT

SEQ ID NO: 221 - protein, 12 aa, synthetic construct
  SITE 1: note = X is acetyl
  SITE 3: note = X is 2-amino-2-methylpropanoic acid
  SITE 7: note = X is N-alpha methyl arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKXXNCXT

SEQ ID NO: 222 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is D-leucine
  MOD_RES 11: note = AMIDATION
  DISULFID 5..10: note = Cys Cys connected through disulfide bond
XTXFCKRXNCXT

SEQ ID NO: 223 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is 2-amino-2-methylpropanoic acid
  MOD_RES 11: note = AMIDATION
  DISULFID 5..10: note = Cys Cys connected through disulfide bond
XTXFCKRXNCXT

SEQ ID NO: 224 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXQ

SEQ ID NO: 225 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXE

SEQ ID NO: 226 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXL

SEQ ID NO: 227 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXG

SEQ ID NO: 228 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXP

SEQ ID NO: 229 - protein, 12 aa, synthetic construct
  SITE 1: note = X is acetyl
  SITE 4: note = X is N-alpha methyl phenylalanine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTAXCKRXNCXT

SEQ ID NO: 230 - protein, 12 aa, synthetic construct
  SITE 1: note = X is acetyl
  SITE 4: note = X is (S)-2-amino-2-methyl-3-phenylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTAXCKRXNCXT

SEQ ID NO: 231 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-aminobenzoic acid
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXFCKRXNCXT

SEQ ID NO: 232 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with InDOTA (In(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 13: note = AMIDATION
  DISULFID 5..10: note = Cys Cys connected through disulfide bond
XTXFCKRXNCXTXX

SEQ ID NO: 233 - protein, 13 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 234 - protein, 13 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 235 - protein, 14 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 13: note = X is 2-(piperidin-4-yl)acetic acid
  SITE 14: note = X is D-lysine with LuDOTA (Lu(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 13: note = AMIDATION
  DISULFID 5..10: note = Cys Cys connected through disulfide bond
XTXFCKRXNCXTXX

SEQ ID NO: 236 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRXNCXT

SEQ ID NO: 237 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is citrulline
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 238 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKRXNCXT

SEQ ID NO: 239 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  MOD_RES 10: note = AMIDATION
  DISULFID 4..9: note = Cys Cys connected through disulfide bond
XXXCKRXNCXT

SEQ ID NO: 240 - protein, 13 aa, synthetic construct
  SITE 1: note = X is Eu(III) - 2,2?,2??,2???-(1,4,7,10-Tetraazacycl ododecane-1,4,7,10-tetrayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 241 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 6-aminohexanoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKRXNCXT

SEQ ID NO: 242 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(4-aminopiperidin-1-yl)acetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKRXNCXT

SEQ ID NO: 243 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 3-aminopropanoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKRXNCXT

SEQ ID NO: 244 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 245 - protein, 13 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 6: note = X is (S)-2-amino-5-guanidino-5-oxopentanoic acid
  SITE 7: note = X is 2-amino-2-methylpropanoic acid
  SITE 10: note = X is tert leucine
  SITE 12: note = X is 2-(piperidin-4-yl)acetic acid
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 4..9: note = Cys Cys connected through disulfide bond
XXXCKXXNCXTXX

SEQ ID NO: 246 - protein, 13 aa, synthetic construct
  SITE 1: note = X is 3-sufamoylpropanoic Acid
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is 2-(piperidin-4-yl)acetic acid
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 4..9: note = Cys Cys connected through disulfide bond
XXXCKRXNCXTXX

SEQ ID NO: 247 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKRXNCXT

SEQ ID NO: 248 - protein, 12 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 4..9: note = Cys Cys connected through disulfide bond
XXXCKRXNCXTX

SEQ ID NO: 249 - protein, 12 aa, synthetic construct
  SITE 1: note = X is 3-sufamoylpropanoic Acid
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 4..9: note = Cys Cys connected through disulfide bond
XXXCKRXNCXTX

SEQ ID NO: 250 - protein, 12 aa, synthetic construct
  SITE 1: note = X is 3-hydroxy propionic acid
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 4..9: note = Cys Cys connected through disulfide bond
XXXCKRXNCXTX

SEQ ID NO: 251 - protein, 12 aa, synthetic construct
  SITE 1: note = X is iso valeric acid (3-methylbutanoic acid)
  SITE 2: note = X is 2-amino-2-methylpropanoic acid
  SITE 7: note = X is 2-amino-2-methylpropanoic acid
  SITE 10: note = X is tert leucine
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 4..9: note = Cys Cys connected through disulfide bond
XXFCKRXNCXTX

SEQ ID NO: 252 - protein, 11 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with APAc linker (2-(4-(amino)piperidin-1-yl)acetic acid) interspersed
  SITE 10: note = X is tert leucine
  MOD_RES 10: note = AMIDATION
  DISULFID 4..9: note = Cys Cys connected through disulfide bond
XXXCKRXNCXT

SEQ ID NO: 253 - protein, 11 aa, synthetic construct
  SITE 1: note = X is 3-sufamoylpropanoic Acid
  SITE 2: note = X is 2-amino-2-methylpropanoic acid
  SITE 3: note = X is (S)-2-amino-3-(4-chlorophenyl)propanoic acid
  SITE 7: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with APAc linker (2-(4-(amino)piperidin-1-yl)acetic acid) interspersed
  SITE 10: note = X is tert leucine
  MOD_RES 10: note = AMIDATION
  DISULFID 4..9: note = Cys Cys connected through disulfide bond
XXXCKRXNCXT

SEQ ID NO: 254 - protein, 11 aa, synthetic construct
  SITE 1: note = X is 3-hydroxy propionic acid
  SITE 2: note = X is 2-amino-2-methylpropanoic acid
  SITE 3: note = X is (S)-2-amino-3-(4-chlorophenyl)propanoic acid
  SITE 7: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with APAc linker (2-(4-(amino)piperidin-1-yl)acetic acid) interspersed
  SITE 10: note = X is tert leucine
  MOD_RES 10: note = AMIDATION
  DISULFID 4..9: note = Cys Cys connected through disulfide bond
XXXCKRXNCXT

SEQ ID NO: 255 - protein, 11 aa, synthetic construct
  SITE 1: note = X is iso valeric acid (3-methylbutanoic acid)
  SITE 2: note = X is 2-amino-2-methylpropanoic acid
  SITE 3: note = X is (S)-2-amino-3-(4-chlorophenyl)propanoic acid
  SITE 7: note = X is lysine with DOTA (2,2 &apos;,2 &apos; &apos;,2 &apos; &apos; &apos;-(1,4,7,10-Tetraazacyclododecane-1,4,7 ,10-tetrayl)tetraacetic acid) attached to side chain with APAc linker (2-(4-(amino)piperidin-1-yl)acetic acid) interspersed
  SITE 10: note = X is tert leucine
  MOD_RES 10: note = AMIDATION
  DISULFID 4..9: note = Cys Cys connected through disulfide bond
XXXCKRXNCXT

SEQ ID NO: 256 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 6: note = X is (S)-2-aminohexanoic acid
  SITE 7: note = X is (S)-2-amino-5-guanidino-5-oxopentanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCXXXNCXT

SEQ ID NO: 257 - protein, 11 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XTXXCKRXNCX

SEQ ID NO: 258 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  DISULFID 5..10: note = Cys Cys connected through disulfide bond
XTXXCKRXNCXT

SEQ ID NO: 259 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is threoninol
  DISULFID 5..10: note = Cys Cys connected through disulfide bond
XTXXCKRXNCXX

SEQ ID NO: 260 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XTXXCKRXNCXX

SEQ ID NO: 261 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 2-hydrazineylethan-1-amine
  DISULFID 5..10: note = Cys Cys connected through disulfide bond
XTXXCKRXNCXX

SEQ ID NO: 262 - protein, 12 aa, synthetic construct
  SITE 1: note = X is iso valeric acid (3-methylbutanoic acid)
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 11: note = AMIDATION
  DISULFID 4..9: note = Cys Cys connected through disulfide bond
XXXCKRXNCXTX

SEQ ID NO: 263 - protein, 13 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRANCXT

SEQ ID NO: 264 - protein, 15 aa, synthetic construct
  SITE 1: note = X is n-hexanoic acid
  SITE 3: note = X is 2-amino-2-methylpropanoic acid
  SITE 8: note = X is 2-amino-2-methylpropanoic acid
  SITE 11: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is D-lysine with LuDOTA (Lu(III) - 2,2 &apos;,2 &apos; &apos;,2 &apos; &apos; &apos;-(1,4,7,10-Tetraazacyc lododecane-1,4,7,10-tetrayl)tetraacetic acid complex) attached to side chain
  MOD_RES 14: note = AMIDATION
  DISULFID 5..10: note = Cys Cys connected through disulfide bond
XTXFCKRXNCXTDXX

SEQ ID NO: 265 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRANCXT

SEQ ID NO: 266 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is 2-amino-2-methylpropanoic acid
  SITE 4: note = X is (S)-2-amino-3-(4-chlorophenyl)propanoic acid
  SITE 7: note = X is (S)-2-amino-5-guanidino-5-oxopentanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKXXNCXT

SEQ ID NO: 267 - protein, 13 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 6: note = X is N-omega-acetyl-L-arginine
  SITE 7: note = X is 2-amino-2-methylpropanoic acid
  SITE 10: note = X is tert leucine
  SITE 12: note = X is 2-(piperidin-4-yl)acetic acid
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 4..9: note = Cys Cys connected through disulfide bond
XXXCKXXNCXTXX

SEQ ID NO: 268 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 6: note = X is (S)-2-aminohexanoic acid
  SITE 7: note = X is N-omega-acetyl-L-arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCXXXNCXT

SEQ ID NO: 269 - protein, 13 aa, synthetic construct
  SITE 1: note = X is 3-hydroxy propionic acid
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is 2-(piperidin-4-yl)acetic acid
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 4..9: note = Cys Cys connected through disulfide bond
XXXCKRXNCXTXX

SEQ ID NO: 270 - protein, 13 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 12: note = X is 2-(piperidin-4-yl)acetic acid
  SITE 13: note = X is D-lysine with DOTA (2,2 &apos;,2 &apos; &apos;,2 &apos; &apos; &apos;-(1,4,7,10-Tetraazacyclododec ane-1,4,7,10-tetrayl)tetraacetic acid) attached to side chain
  MOD_RES 12: note = AMIDATION
  DISULFID 4..9: note = Cys Cys connected through disulfide bond
XXXCKRXNCXTXX

SEQ ID NO: 271 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is 2-amino-2-methylpropanoic acid
  SITE 4: note = X is (S)-2-amino-3-(4-chlorophenyl)propanoic acid
  SITE 7: note = X is N-omega-acetyl-L-arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XXXXCKXXNCXT

SEQ ID NO: 272 - protein, 11 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is 2,2-dimethylpropan-1-amine
  DISULFID 5..10: note = Cys Cys connected through disulfide bond
XTXFCKRXNCX

SEQ ID NO: 273 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 12: note = X is butan-1-amine
  DISULFID 5..10: note = Cys Cys connected through disulfide bond
XTXFCKRXNCXX

SEQ ID NO: 274 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XTXFCKRXNCXX

SEQ ID NO: 275 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  SITE 12: note = X is 4-aminobutanoic acid
  MOD_RES 11: note = AMIDATION
  DISULFID 5..10: note = Cys Cys connected through disulfide bond
XTXFCKRXNCXX

SEQ ID NO: 276 - protein, 11 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XTXFCKRXNCX

SEQ ID NO: 277 - protein, 11 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is (S)-2-amino-4,4-dimethylpentanamide
  MOD_RES 10: note = AMIDATION
  DISULFID 5..10: note = Cys Cys connected through disulfide bond
XTXFCKRXNCX

SEQ ID NO: 278 - protein, 11 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  DISULFID 5..10: note = Cys Cys connected through disulfide bond
XTXFCKRXNCX

SEQ ID NO: 279 - protein, 13 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 2-hydrazineylethan-1-amine
  SITE 13: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 5..10: note = Cys Cys connected through disulfide bond
XTXXCKRXNCXXX

SEQ ID NO: 280 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XTXXCKRXNCX

SEQ ID NO: 281 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XXXXCKRXNCX

SEQ ID NO: 282 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCKRXNCX

SEQ ID NO: 283 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 2-hydrazineylethan-1-amine
  DISULFID 5..10: note = Cys Cys connected through disulfide bond
XTXXCKRXNCXX

SEQ ID NO: 284 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 2-hydrazineylethan-1-amine
  DISULFID 5..10: note = Cys Cys connected through disulfide bond
XXXXCKRXNCXX

SEQ ID NO: 285 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXFCKRXNCX

SEQ ID NO: 286 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 8: note = X is 2-amino-2-methylpropanoic acid
  SITE 11: note = X is tert leucine
  SITE 12: note = X is 2-hydrazineylethan-1-amine
  DISULFID 5..10: note = Cys Cys connected through disulfide bond
XTXFCKRXNCXX

SEQ ID NO: 287 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 8: note = X is 2-amino-2-methylpropanoic acid
  SITE 11: note = X is tert leucine
  SITE 12: note = X is 2-hydrazineylethan-1-amine
  DISULFID 5..10: note = Cys Cys connected through disulfide bond
XXXFCKRXNCXX

SEQ ID NO: 288 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  SITE 13: note = X is 2-hydrazineylethan-1-amine
  DISULFID 6..11: note = Cys Cys connected through disulfide bond
XXTXFCKRXNCXX

SEQ ID NO: 289 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XTXFCKRXNCX

SEQ ID NO: 290 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 8: note = X is 2-amino-2-methylpropanoic acid
  SITE 11: note = X is tert leucine
  MOD_RES 10: note = AMIDATION
  DISULFID 5..10: note = Cys Cys connected through disulfide bond
XXXFCKRXNCX

SEQ ID NO: 291 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XTXFCKRXNCXX

SEQ ID NO: 292 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XXXFCKRXNCXX

SEQ ID NO: 293 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 9: note = X is 2-amino-2-methylpropanoic acid
  SITE 12: note = X is tert leucine
  SITE 13: note = X is 3-aminopropanoic acid
  MOD_RES 12: note = AMIDATION
  DISULFID 6..11: note = Cys Cys connected through disulfide bond
XXTXFCKRXNCXX

SEQ ID NO: 294 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 13: note = X is 2-hydrazineylethan-1-amine
  DISULFID 6..11: note = Cys Cys connected through disulfide bond
XXTXXCKRXNCXX

SEQ ID NO: 295 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XTXXCKRXNCXX

SEQ ID NO: 296 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 4-(aminomethyl)benzoic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 3-aminopropanoic acid
  MOD_RES 11: note = AMIDATION
  DISULFID 5..10: note = Cys Cys connected through disulfide bond
XXXXCKRXNCXX

SEQ ID NO: 297 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 13: note = X is 3-aminopropanoic acid
  MOD_RES 12: note = AMIDATION
  DISULFID 6..11: note = Cys Cys connected through disulfide bond
XXTXXCKRXNCXX

SEQ ID NO: 298 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is (S)-2-amino-3-hydroxy-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 299 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is (S)-2-amino-3-hydroxy-2-methylpropanoic acid
  SITE 5: note = X is (S)-2-amino-3-(4-chlorophenyl)propanoic acid
  SITE 9: note = X is (S)-2-amino-3-hydroxy-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 300 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is (S)-2-amino-3-hydroxy-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 301 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is N-alpha methyl lysine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 302 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is N-alpha methyl leucine
  MOD_RES 12: note = AMIDATION
  DISULFID 6..11: note = Cys Cys connected through disulfide bond
XXTXXCKRXNCXT

SEQ ID NO: 303 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is N-alpha methyl arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 304 - protein, 14 aa, synthetic construct
  SITE 1: note = X is acetyl
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 13: note = X is 2-hydrazineylethan-1-amine
  SITE 14: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 6..11: note = Cys Cys connected through disulfide bond
XXTXXCKRXNCXXX

SEQ ID NO: 305 - protein, 13 aa, synthetic construct
  SITE 1: note = X is acetyl
  SITE 2: note = X is 6-(aminomethyl)nicotinic acid
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  SITE 12: note = X is 2-hydrazineylethan-1-amine
  SITE 13: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 5..10: note = Cys Cys connected through disulfide bond
XXXXCKRXNCXXX

SEQ ID NO: 306 - protein, 12 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  SITE 11: note = X is 2-hydrazineylethan-1-amine
  SITE 12: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 4..9: note = Cys Cys connected through disulfide bond
XXXCKRXNCXXX

SEQ ID NO: 307 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 308 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 309 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 13: note = X is N1,N1-dimethylethane-1,2-diamine
  DISULFID 6..11: note = Cys Cys connected through disulfide bond
XXTXXCKRXNCXX

SEQ ID NO: 310 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 13: note = X is N1-methylethane-1,2-diamine
  DISULFID 6..11: note = Cys Cys connected through disulfide bond
XXTXXCKRXNCXX

SEQ ID NO: 311 - protein, 16 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is 1,13-diamino-4,7,10-trioxatridecan-succinamic acid (1-amino-15-oxo-4,7,10-trioxa-14-azaoctadecan-18-oic acid)
  SITE 16: note = X is L-lysine with D-biotin attached to side chain
  MOD_RES 15: note = AMIDATION
  DISULFID 6..11: note = Cys Cys connected through disulfide bond
XXTXXCKRXNCXTXXX

SEQ ID NO: 312 - protein, 13 aa, synthetic construct
  SITE 1: note = X is Ga(III) - 2,2?,2??,2???-(1,4,7,10-Tetraazacycl ododecane-1,4,7,10-tetrayl)tetraacetic acid complex
  SITE 2: note = X is 1,13-diamino-4,7,10-trioxatridecan-succinamic acid (1-amino-15-oxo-4,7,10-trioxa-14-azaoctadecan-18-oic acid)
  SITE 4: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXFCKRANCXT

SEQ ID NO: 313 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is (R)-2-amino-3-hydroxy-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 314 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is (R)-2-amino-3-hydroxy-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 315 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is N-alpha methyl threonine
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXXAXCKRXNCXT

SEQ ID NO: 316 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 10: note = X is N-alpha methyl asparagine
  SITE 12: note = X is tert leucine
  MOD_RES 12: note = AMIDATION
  DISULFID 6..11: note = Cys Cys connected through disulfide bond
XXTXXCKRAXCXT

SEQ ID NO: 317 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 13: note = X is N-alpha methyl threonine
  MOD_RES 12: note = AMIDATION
  DISULFID 6..11: note = Cys Cys connected through disulfide bond
XXTXXCKRXNCVX

SEQ ID NO: 318 - protein, 14 aa, synthetic construct
  SITE 1: note = X is D-biotin (5-((3aS,4S,6aR)-2-oxohexahydro-1H-th ieno[3,4-d]imidazol-4-yl)pentanoic acid)
  SITE 2: note = X is 1,13-diamino-4,7,10-trioxatridecan-succinamic acid (1-amino-15-oxo-4,7,10-trioxa-14-azaoctadecan-18-oic acid)
  SITE 3: note = X is 1,13-diamino-4,7,10-trioxatridecan-succinamic acid (1-amino-15-oxo-4,7,10-trioxa-14-azaoctadecan-18-oic acid)
  SITE 5: note = X is 2-amino-2-methylpropanoic acid
  SITE 6: note = X is (S)-2-amino-3-(4-chlorophenyl)propanoic acid
  SITE 10: note = X is 2-amino-2-methylpropanoic acid
  SITE 13: note = X is tert leucine
  MOD_RES 13: note = AMIDATION
  DISULFID 7..12: note = Cys Cys connected through disulfide bond
XXXTXXCKRXNCXT

SEQ ID NO: 319 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is L-lysine with D-biotin attached to side chain
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 320 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 321 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is D-glutamic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 322 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTEXCKRXNCXT

SEQ ID NO: 323 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is (S)-2-amino-2-methylsuccinic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 324 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 12: note = X is tert leucine
  MOD_RES 12: note = AMIDATION
  DISULFID 6..11: note = Cys Cys connected through disulfide bond
XXTXXCKRENCXT

SEQ ID NO: 325 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is D-glutamic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 326 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is (S)-2-amino-2-methylsuccinic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 327 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is (R)-2-amino-2-methylsuccinic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 328 - protein, 12 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 6: note = X is N-omega-methyl-L-arginine
  SITE 7: note = X is 2-amino-2-methylpropanoic acid
  SITE 10: note = X is tert leucine
  SITE 11: note = X is 2-hydrazineylethan-1-amine
  SITE 12: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 4..9: note = Cys Cys connected through disulfide bond
XXXCKXXNCXXX

SEQ ID NO: 329 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is (S)-2,5-diaminopentanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 330 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is homo arginine (N6-carbamimidoyl-L-lysine)
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 331 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is N-epsilon acetyl lysine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 332 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is homo glutamine ((S)-2,6-diamino-6-oxohexanoic acid)
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXRXNCXT

SEQ ID NO: 333 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is (S)-2,5-diaminopentanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 334 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is homo arginine (N6-carbamimidoyl-L-lysine)
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 335 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is N-epsilon acetyl lysine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 336 - protein, 12 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 5: note = X is epsilon-methyl lysine
  SITE 7: note = X is 2-amino-2-methylpropanoic acid
  SITE 10: note = X is tert leucine
  SITE 11: note = X is 2-hydrazineylethan-1-amine
  SITE 12: note = X is 2,2 &apos;,2 &apos; &apos;,2 &apos; &apos; &apos;-(1,4,7,10-Tetraazacyclododecane-1,4,7,10-tetrayl)te traacetic acid
  DISULFID 4..9: note = Cys Cys connected through disulfide bond
XXXCXRXNCXXX

SEQ ID NO: 337 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is homo glutamine ((S)-2,6-diamino-6-oxohexanoic acid)
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 338 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is (R)-2-amino-2-methylsuccinic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKRXNCXT

SEQ ID NO: 339 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is N-omega-(ethylcarbamoyl)-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 340 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is (S)-2-amino-3-(3-carbamimidoylureido)propanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCKXXNCXT

SEQ ID NO: 341 - protein, 12 aa, synthetic construct
  SITE 1: note = X is acetyl
  SITE 3: note = X is 2-amino-2-methylpropanoic acid
  SITE 4: note = X is (S)-2-amino-3-(3-fluorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 342 - protein, 12 aa, synthetic construct
  SITE 1: note = X is acetyl
  SITE 3: note = X is 2-amino-2-methylpropanoic acid
  SITE 4: note = X is (S)-2-amino-3-(3-cyanophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 343 - protein, 12 aa, synthetic construct
  SITE 1: note = X is acetyl
  SITE 3: note = X is 2-amino-2-methylpropanoic acid
  SITE 4: note = X is (S)-2-amino-3-(m-tolyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 344 - protein, 12 aa, synthetic construct
  SITE 1: note = X is acetyl
  SITE 3: note = X is 2-amino-2-methylpropanoic acid
  SITE 4: note = X is (S)-2-amino-3-(4-fluorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 345 - protein, 12 aa, synthetic construct
  SITE 1: note = X is acetyl
  SITE 3: note = X is 2-amino-2-methylpropanoic acid
  SITE 4: note = X is S-benzyl cysteine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 346 - protein, 12 aa, synthetic construct
  SITE 1: note = X is acetyl
  SITE 3: note = X is 2-amino-2-methylpropanoic acid
  SITE 4: note = X is (S)-2-amino-3-(3-(trifluoromethyl)phenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 347 - protein, 12 aa, synthetic construct
  SITE 1: note = X is acetyl
  SITE 3: note = X is 2-amino-2-methylpropanoic acid
  SITE 4: note = X is (S)-2-amino-3-(4-(trifluoromethyl)phenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 348 - protein, 12 aa, synthetic construct
  SITE 1: note = X is acetyl
  SITE 3: note = X is 2-amino-2-methylpropanoic acid
  SITE 4: note = X is homo phenylalanine ((S)-2-amino-4-phenylbutanoic acid)
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 349 - protein, 10 aa, synthetic construct
  SITE 1: note = X is alpha hydroxy isobutyric acid (2-hydroxy-2-methylpropanoic acid)
  SITE 2: note = X is (S)-2-amino-3-(4-chlorophenyl)propanoic acid
  SITE 6: note = X is 2-amino-2-methylpropanoic acid
  SITE 9: note = X is tert leucine
  MOD_RES 9: note = AMIDATION
  DISULFID 3..8: note = Cys Cys connected through disulfide bond
XXCKRXNCXT

SEQ ID NO: 350 - protein, 12 aa, synthetic construct
  SITE 1: note = X is acetyl
  SITE 3: note = X is 2-amino-2-methylpropanoic acid
  SITE 4: note = X is (S)-2-amino-3-(4-cyanophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 351 - protein, 12 aa, synthetic construct
  SITE 1: note = X is acetyl
  SITE 3: note = X is 2-amino-2-methylpropanoic acid
  SITE 4: note = X is (S)-2-amino-3-(p-tolyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXNCXT

SEQ ID NO: 352 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(6-chloro-1H-indol-3-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 353 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-2-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 354 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 355 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-(trifluoromethyl)phenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 356 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-(trifluoromethyl)phenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 357 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-fluorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 358 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(3-cyanophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 359 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(m-tolyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 360 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-fluorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 361 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCKXXNCX

SEQ ID NO: 362 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXRXNCX

SEQ ID NO: 363 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 11: note = AMIDATION
XXTXXSKRXNSX

SEQ ID NO: 364 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 9: note = X is 2-amino-2-methylpropanoic acid
  SITE 12: note = X is tert leucine
  MOD_RES 11: note = AMIDATION
XXTXXAKRXNAX

SEQ ID NO: 365 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 6: note = X is homo serine
  SITE 9: note = X is 2-amino-2-methylpropanoic acid
  SITE 11: note = X is homo serine
  SITE 12: note = X is tert leucine
  MOD_RES 11: note = AMIDATION
XXTXXXKRXNXX

SEQ ID NO: 366 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 367 - protein, 12 aa, synthetic construct
  SITE 1: note = X is Ga(III) - 2,2?,2??,2???-(1,4,7,10-Tetraazacycl ododecane-1,4,7,10-tetrayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 368 - protein, 13 aa, synthetic construct
  SITE 1: note = X is Ga(III) - 2,2?,2??,2???-(1,4,7,10-Tetraazacycl ododecane-1,4,7,10-tetrayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 369 - protein, 12 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 370 - protein, 13 aa, synthetic construct
  SITE 1: note = X is In(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 371 - protein, 12 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 11: note = AMIDATION
  DISULFID 6..11: note = Cys Cys connected through disulfide bond
XXTXXCXXXNCX

SEQ ID NO: 372 - protein, 13 aa, synthetic construct
  SITE 1: note = X is Lu(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 373 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is tert leucine
  SITE 4: note = X is 2-amino-2-methylpropanoic acid
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXXXFCKRXNCXT

SEQ ID NO: 374 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 3: note = X is tert leucine
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXXXXCXXXNCXT

SEQ ID NO: 375 - protein, 12 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 6: note = X is S-methyl cysteine
  SITE 9: note = X is 2-amino-2-methylpropanoic acid
  SITE 12: note = X is tert leucine
  MOD_RES 11: note = AMIDATION
  CROSSLNK 6..11: note = Smc Cys connected through a methylene bond (S-CH2-S)
XXTXXXKRXNCX

SEQ ID NO: 376 - protein, 10 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  SITE 10: note = X is tert leucine
  MOD_RES 9: note = AMIDATION
  DISULFID 4..9: note = Cys Cys connected through disulfide bond
XXXCKRXNCX

SEQ ID NO: 377 - protein, 9 aa, synthetic construct
  SITE 1: note = X is acetyl
  SITE 2: note = X is (S)-2-amino-3-(4-chlorophenyl)propanoic acid
  SITE 6: note = X is 2-amino-2-methylpropanoic acid
  SITE 9: note = X is tert leucine
  MOD_RES 8: note = AMIDATION
  DISULFID 3..8: note = Cys Cys connected through disulfide bond
XXCKRXNCX

SEQ ID NO: 378 - protein, 9 aa, synthetic construct
  SITE 1: note = X is acetyl
  SITE 2: note = X is 2-amino-2-methylpropanoic acid
  SITE 3: note = X is (S)-2-amino-3-(4-chlorophenyl)propanoic acid
  SITE 7: note = X is 2-amino-2-methylpropanoic acid
  MOD_RES 8: note = AMIDATION
  DISULFID 4..9: note = Cys Cys connected through disulfide bond
XXXCKRXNC

SEQ ID NO: 379 - protein, 8 aa, synthetic construct
  SITE 1: note = X is acetyl
  SITE 2: note = X is (S)-2-amino-3-(4-chlorophenyl)propanoic acid
  SITE 6: note = X is 2-amino-2-methylpropanoic acid
  MOD_RES 7: note = AMIDATION
  DISULFID 3..8: note = Cys Cys connected through disulfide bond
XXCKRXNC

SEQ ID NO: 380 - protein, 12 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCKRXMCXT

SEQ ID NO: 381 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega,N-omega dimethyl arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 382 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is N-epsilon dimethyl lysine (N6,N6-dimethyl-L-lysine)
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 383 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is N-epsilon trimethyl lysine ((S)-5-amino-5-carboxy-N,N,N-trimethylpentan-1-aminium)
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 384 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is N-epsilon isopropyl lysine (N6-isopropyl-L-lysine)
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 385 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-nitro-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 386 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is D-alanine
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 387 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(5-chloro-1H-indol-3-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 388 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(5-bromo-1H-indol-3-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 389 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(quinolin-3-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 390 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-2-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 391 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(pyridin-3-yl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 392 - protein, 15 aa, synthetic construct
  SITE 1: note = X is acetyl
  SITE 3: note = X is 2-amino-2-methylpropanoic acid
  SITE 4: note = X is (S)-2-amino-3-(4-chlorophenyl)propanoic acid
  SITE 6: note = X is epsilon-methyl lysine
  SITE 7: note = X is N-omega-methyl-L-arginine
  SITE 8: note = X is 2-amino-2-methylpropanoic acid
  SITE 11: note = X is tert leucine
  SITE 13: note = X is 1,13-diamino-4,7,10-trioxatridecan-succinamic acid (1-amino-15-oxo-4,7,10-trioxa-14-azaoctadecan-18-oic acid)
  SITE 14: note = X is 1,13-diamino-4,7,10-trioxatridecan-succinamic acid (1-amino-15-oxo-4,7,10-trioxa-14-azaoctadecan-18-oic acid)
  SITE 15: note = X is 2-(6-amino-3-imino-4,5-disulfo-3H-xanthen-9-yl )-5-((3-(1-((R)-5-amino-6-(((S)-4-amino-1-(((S)-1-carboxy- 2-(1H-imidazol-4-yl)ethyl)amino)-1,4-dioxobutan-2-yl)amino )-6-oxohexyl)-1,9-dihydro-8H-dibenzo[b,f][1,2,3]triazolo[4 ,5-d]azocin-8-yl)-3-oxopropyl)carbamoyl)benzoic acid
  MOD_RES 14: note = AMIDATION
  DISULFID 5..10: note = Cys Cys connected through disulfide bond
XTXXCXXXNCXTXXX

SEQ ID NO: 393 - protein, 14 aa, synthetic construct
  SITE 1: note = X is 2-(6-amino-3-imino-4,5-disulfo-3H-xanthen-9-yl )-5-((3-(1-(5-carboxypentyl)-1,9-dihydro-8H-dibenzo[b,f][1 ,2,3]triazolo[4,5-d]azocin-8-yl)-3-oxopropyl)carbamoyl)ben zoic acid
  SITE 2: note = X is 1,13-diamino-4,7,10-trioxatridecan-succinamic acid (1-amino-15-oxo-4,7,10-trioxa-14-azaoctadecan-18-oic acid)
  SITE 3: note = X is 1,13-diamino-4,7,10-trioxatridecan-succinamic acid (1-amino-15-oxo-4,7,10-trioxa-14-azaoctadecan-18-oic acid)
  SITE 5: note = X is 2-amino-2-methylpropanoic acid
  SITE 6: note = X is (S)-2-amino-3-(4-chlorophenyl)propanoic acid
  SITE 8: note = X is epsilon-methyl lysine
  SITE 9: note = X is N-omega-methyl-L-arginine
  SITE 10: note = X is 2-amino-2-methylpropanoic acid
  SITE 13: note = X is tert leucine
  MOD_RES 13: note = AMIDATION
  DISULFID 7..12: note = Cys Cys connected through disulfide bond
XXXTXXCXXXNCXT

SEQ ID NO: 394 - protein, 12 aa, synthetic construct
  SITE 1: note = X is acetyl
  SITE 3: note = X is 2-amino-2-methylpropanoic acid
  SITE 4: note = X is (S)-2-amino-3-(4-hydroxy-3-iodophenyl)propanoic acid
  SITE 6: note = X is epsilon-methyl lysine
  SITE 7: note = X is N-omega-methyl-L-arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCXXXNCXT

SEQ ID NO: 395 - protein, 13 aa, synthetic construct
  SITE 1: note = X is 2,2 &apos;,2 &apos; &apos;,2 &apos; &apos; &apos;-(1,10-dioxa-4,7,13,16-tetraazacyclooctadecane-4,7,1 3,16-tetrayl)tetraacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 396 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega,N-omega &apos;-dimethyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 397 - protein, 12 aa, synthetic construct
  SITE 1: note = X is 2-(piperidin-4-yl)acetic acid
  SITE 3: note = X is 2-amino-2-methylpropanoic acid
  SITE 4: note = X is (S)-2-amino-3-(4-hydroxy-3-iodophenyl)propanoic acid
  SITE 6: note = X is epsilon-methyl lysine
  SITE 7: note = X is N-omega-methyl-L-arginine
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXXCXXXNCXT

SEQ ID NO: 398 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-delta,N-omega dimethyl arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 399 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-delta,N-omega,N-omega &apos; trimethyl arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 400 - protein, 13 aa, synthetic construct
  SITE 1: note = X is 4-(4-carboxybutanamido)-6-((16-((6-carboxypyri din-2-yl)methyl)-1,4,10,13-tetraoxa-7,16-diazacyclooctadec an-7-yl)methyl)picolinic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 401 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-delta methyl arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 402 - protein, 13 aa, synthetic construct
  SITE 1: note = X is 2-(4,7,10-tris(2-amino-2-oxoethyl)-1,4,7,10-te traazacyclododecan-1-yl)acetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 403 - protein, 11 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  DISULFID 6..11: note = Cys Cys connected through disulfide bond
XXTXXCXXXNC

SEQ ID NO: 404 - protein, 13 aa, synthetic construct
  SITE 1: note = X is Bi(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 405 - protein, 13 aa, synthetic construct
  SITE 1: note = X is La(III) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 406 - protein, 13 aa, synthetic construct
  SITE 1: note = X is Pb(II) - 2,2 &apos;,2 &apos; &apos;,2 &apos; &apos; &apos;-(1,4,7,10-Tetraazacyclododecane-1,4,7,10-tet rayl)tetraacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 407 - protein, 13 aa, synthetic construct
  SITE 1: note = X is Bi(III) - 2-(4,7,10-tris(2-amino-2-oxoethyl)-1 ,4,7,10-tetraazacyclododecan-1-yl)acetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 408 - protein, 13 aa, synthetic construct
  SITE 1: note = X is In(III) - 2-(4,7,10-tris(2-amino-2-oxoethyl)-1 ,4,7,10-tetraazacyclododecan-1-yl)acetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 409 - protein, 13 aa, synthetic construct
  SITE 1: note = X is Pb(II) - 2-(4,7,10-tris(2-amino-2-oxoethyl)-1, 4,7,10-tetraazacyclododecan-1-yl)acetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 410 - protein, 13 aa, synthetic construct
  SITE 1: note = X is 2,2 &apos;,2 &apos; &apos;-(10-(2-amino-2-oxoe thyl)-1,4,7,10-tetraazacyclododecane-1,4,7-triyl)triacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 411 - protein, 13 aa, synthetic construct
  SITE 1: note = X is Pb(II) - 2,2 &apos;,2 &apos; &apos;-(10-(2-ami no-2-oxoethyl)-1,4,7,10-tetraazacyclododecane-1,4,7-triyl) triacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 412 - protein, 13 aa, synthetic construct
  SITE 1: note = X is Bi(III) - 2,2 &apos;,2 &apos; &apos;-(10-(2-am ino-2-oxoethyl)-1,4,7,10-tetraazacyclododecane-1,4,7-triyl )triacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 413 - protein, 13 aa, synthetic construct
  SITE 1: note = X is In(III) - 2,2 &apos;,2 &apos; &apos;-(10-(2-am ino-2-oxoethyl)-1,4,7,10-tetraazacyclododecane-1,4,7-triyl )triacetic acid complex
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  MOD_RES 12: note = AMIDATION
  DISULFID 6..11: note = Cys Cys connected through disulfide bond
XXTXXCXXXNCXT

SEQ ID NO: 414 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  SITE 13: note = X is 2-hydrazineylethan-1-amine
  DISULFID 6..11: note = Cys Cys connected through disulfide bond
XXTXFCXXXNCXX

SEQ ID NO: 415 - protein, 13 aa, synthetic construct
  SITE 1: note = X is 2,2 &apos;,2 &apos; &apos;,2 &apos; &apos; &apos;-(1,4,7,10-Tetraazacyclododecane-1,4,7,10-tetrayl)te traacetic acid
  SITE 2: note = X is 2-(piperidin-4-yl)acetic acid
  SITE 4: note = X is 2-amino-2-methylpropanoic acid
  SITE 5: note = X is (S)-2-amino-3-(4-chlorophenyl)propanoic acid
  SITE 7: note = X is epsilon-methyl lysine
  SITE 8: note = X is N-omega-methyl-L-arginine
  SITE 9: note = X is 2-amino-2-methylpropanoic acid
  SITE 12: note = X is tert leucine
  SITE 13: note = X is 3-aminopropanoic acid
  MOD_RES 12: note = AMIDATION
  DISULFID 6..11: note = Cys Cys connected through disulfide bond
XXTXXCXXXNCXX

SEQ ID NO: 416 - protein, 12 aa, synthetic construct
  SITE 1: note = X is acetyl
  SITE 3: note = X is 2-amino-2-methylpropanoic acid
  SITE 8: note = X is 2-amino-2-methylpropanoic acid
  SITE 11: note = X is tert leucine
  MOD_RES 11: note = AMIDATION
  DISULFID 5..10: note = Cys Cys connected through disulfide bond
XTXFCKRXNCXT

(19) United States
(12) Patent Application Publication
(10) Pub. No.: US 2026/0083865 A1
(43) Pub. Date: Mar. 26, 2026

(54) PROSTATE SPECIFIC MEMBRANE ANTIGEN (PSMA) LIGANDS AND USE THEREOF

(71) Applicant: 3B Pharmaceuticals GMBH, Berlin (DE)

(72) Inventors: Anne BREDENBECK, Berlin (DE); Christian Haase, Berlin (DE); Dirk Zboralski, Berlin (DE); Eberhard Schneider, Michendorf OT Wildenbruch (DE); Ina Wilkening, Berlin (DE); Jan Ungewiss, Berlin (DE); Jessica Wahsner-Teschner, Köln (DE); Judith Weber, Berlin (DE); Mattias Paschke, Berlin (DE); Naowras Al-Obaidi, Berlin (DE)

(73) Assignee: 3B Pharmaceuticals GMBH, Berlin (DE)

(21) Appl. No.: 19/109,312

(22) PCT Filed: Sep. 6, 2023
(86) PCT No.: PCT/EP2023/074507

§ 371 Date: Mar. 6, 2025

Related U.S. Application Data
(60) Provisional application No. 63/374,841, filed on Sep. 7, 2022.

(51) Int. Cl. A61K 51/08; A61P 35/00

(52) U.S. Cl. CPC A61K 51/088; A61P 35/00; A61K 2121/00; A61K 2123/00

(57) ABSTRACT

The present disclosure is related to a compound of Formula (I)

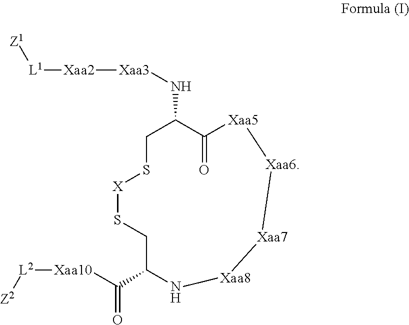

### FIELD OF INVENTION

The present invention is related to a chemical compound; a ligand of prostate specific membrane antigen (PSMA); a composition comprising the compound; the compound, or the composition, respectively, for use in a method for the diagnosis of a disease; the compound, or the composition, respectively, for use in a method for the treatment of a disease; the compound, or the composition, respectively, for use in a method of diagnosis and treatment of a disease which is also referred to as “thera(g)nosis” or “thera(g)nostics”; the compound, or the composition, respectively, for use in a method for delivering a therapeutically active nuclide or a diagnostically active nuclide to a PSMA expressing tissue; a method for the diagnosis of a disease using the compound, or the composition, respectively; a method for the treatment of a disease using the compound, or the composition, respectively; a method for the diagnosis and treatment of a disease which is also referred to as “thera(g)nosis” or “thera(g)nostics, using the compound or the composition, respectively; a method for the delivery of a therapeutically active nuclide or a diagnostically active nuclide to a PSMA expressing tissue using the compound, or the composition, respectively.

### BACKGROUND

Prostate cancer (PCa) is one of the most frequently diagnosed cancers in men, and is the second most common cause of cancer-related death after lung cancer in males. The risk of developing prostate cancer increases dramatically with age, particularly for men over 50. With an aging population and increases in life expectancy that have marked the last thirty years, the incidence rate of prostate cancer in the United States is approaching one in six men.

Early diagnosis and successful treatment of prostate cancer continues to be a major clinical challenge. Apart from new technologies that accurately detect prostatic lesions, understanding significant molecular cascades during prostate carcinogenesis, metastasis, and drug resistance are critical for the development of new therapeutic agents and intervention strategies. A molecular prostate cancer hallmark is the aberrant expression of the transmembrane glycoprotein prostate specific membrane antigen (PSMA) at the plasma membrane of almost every prostatic neoplasia. PSMA's expression profile on prostate cancers and its limited expression in normal organs suggest that it might play an important role in prostate cancer and represents an attractive target for pharmacological intervention. The ability to target PSMA for the specific delivery of anti-cancer compounds could be useful in the treatment of cancer and other diseases and conditions that express PSMA.

PSMA is a trans-membrane, 750 amino acid type II glycoprotein (SEQ ID NO: 1) that has abundant and restricted expression on the surface of prostate cancers, particularly in androgen-independent, advanced and metastatic disease. The latter is important since almost all prostate cancers become androgen independent over time. PSMA possesses the criteria of a promising target for therapy, i.e., abundant and restricted (to prostate) expression at all stages of the disease, presentation at the cell surface but not shed into the circulation, and association with enzymatic or signaling activity. Metastatic spread and disease progression under androgen deprivation therapy signify the onset of metastatic castration resistant prostate cancer (mCRPC)—the lethal form of the disease, which severely deteriorates the quality of life of patients. Although therapies are approved for mCRPC, their survival benefit is generally limited to less than 6 months.

PSMA is also present in the endothelial cells of the neovasculature of non-prostate tumors including kidney, lung, stomach, colon, and breast where it may facilitate endothelial cell sprouting and invasion through its regulation of lytic proteases that have the ability to cleave the extracellular matrix.

The selective targeting of cancer cells with radiopharmaceuticals, either for imaging or therapeutic purposes is challenging. A variety of radionuclides are known to be useful for radio-imaging or cancer radiotherapy.

Despite the significant advancement in the diagnosis and treatment of cancer, improved therapies are still being sought. There is a clinical need for improved therapies for the treatment of cancer, such as prostate cancer, including therapies which can provide a more effective and/or sustained response.

### SUMMARY OF THE INVENTION

One aspect of the present disclosure pertains a compound of Formula (I)

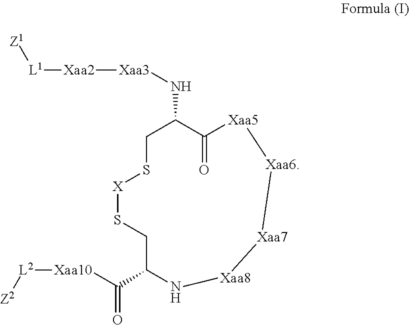

wherein
X is selected from the group consisting of bond and —CH₂—;
Z¹ is selected from the group consisting of chelator and NT;
   NT is selected from the group consisting of H, Ac, Hex, HPA, HO-Succinyl, SaPr, Iva, HYDAc, Bio, nBuCAyl, AF488Ahx, and Hib;
L¹ is selected from the group consisting of a bond and -(Xaa1)ₖ-,
   k is selected from the group consisting of 1, 2, and 3,
   Xaa1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI),

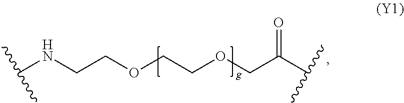

g is an integer selected from the group consisting of 0-23,
wherein,
if k=1, Xaa1 is covalently bound to Z¹ and covalently bound to Xaa2,
if k=2, a first of the two Xaa1 is covalently bound to Z¹ and covalently bound to a second of the two Xaa1, and the second of the two Xaa1 is covalently bound to the first of the two Xaa1 and covalently bound to Xaa2,
if k=3, a first of the three Xaa1 is covalently bound to Z¹ and covalently bound to a second of the three Xaa1, the second of the three Xaa1 is covalently bound to the first of the three Xaa1 and covalently bound to a third of the three Xaa1, and the third of the three Xaa1 is covalently bound to the second of the three Xaa1 and covalently bound to Xaa2;
wherein when L¹ is bond, then Z¹ is NT;
Xaa2 is selected from the group consisting of Formula (II) and Formula (III):

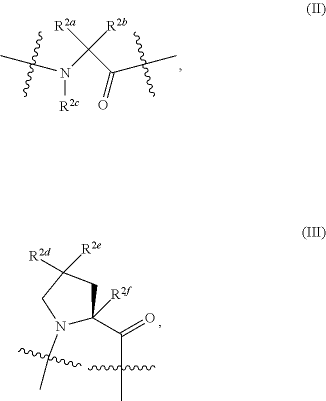

wherein R^{2a} is selected from the group consisting of H, (C₁-C₆)alkyl, and CH₂R^{2g};
   R^{2g} is selected from the group consisting of OH, and CO₂H;
R^{2b} is selected from the group consisting of H and (C₁-C₆)alkyl;
or alternatively, R^{2a} and R^{2b} can together form a 5 or 6 membered carbocycle or heterocycle,
R^{2c} is selected from the group consisting of H and CH₃;
R^{2d} is selected from the group consisting of H, F, and OH;
R^{2e} is selected from the group consisting of H and F;
R^{2f} is selected from the group consisting of H and CH₃;
with the proviso that Xaa2 can be absent when L¹ is bond and NT is Hib;
Xaa3 is selected from Formula (IV):

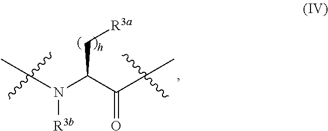

wherein R^{3a} is selected from the group consisting of aryl, (C₅-C₆)heteroaryl, indol-3-yl, 6-chloro-1H-indol-3-yl, and —S—CH₂-phenyl; and wherein said aryl or said heteroaryl ring of R^{3a} is optionally substituted by 1, 2, or 3 substituents independently selected from the group consisting of halogen, (C₁-C₆)alkyl, CN, OH, —O(C₁-C₃)alkyl, wherein said (C₁-C₆)alkyl may optionally be substituted by one or more fluorine;
R^{3b} is selected from the group consisting of H and CH₃;
h is selected from the group consisting of 1 and 2;
Xaa5 is selected from the group consisting of Formula (Va), Formula (Vb), Hgn, lys, Nle, Tap, Aph, and Gln:

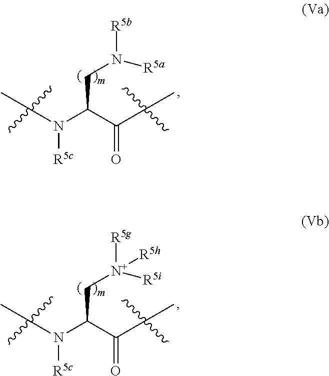

wherein R^{5a} is selected from the group consisting of H, (C₁-C₆)alkyl, Ac, C(═NR^{5d})NR^{5e}R^{5f}, and Bio;
   R^{5d} is selected from the group consisting of H and CH₃;
   R^{5e} and R^{5f} are independently selected from the group consisting of H and (C₁-C₆)alkyl;
R^{5b} is selected from the group consisting of H and (C₁-C₆)alkyl;
R^{5c} is selected from the group consisting of H and CH₃;
m is selected from the group consisting of 2, 3, 4, and 5; and
R^{5g}, R^{5h}, and R⁵ⁱ are independently selected from the group consisting of (C₁-C₆)alkyl;
Xaa6 is selected from the group consisting of Formula (VI), Formula (VII), Nle, and arg:

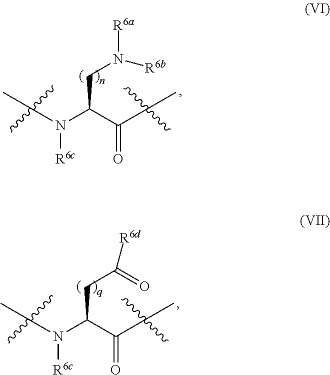

wherein R^{6a} is selected from the group consisting of H, C(═NR^{6e})NR^{6f}R^{6g}, C(═O)R^{6h}, and pyridyl;
   R^{6e} is selected from the group consisting of H and CH₃;
   R^{6f} is selected from the group consisting of H, (C₁-C₆)alkyl, Ac, NO₂, and C(═O)NR⁶ⁱR^{6j};
      R⁶ⁱ and R^{6j} are independently selected from the group consisting of H and (C₁-C₂)alkyl;
   R^{6g} is selected from the group consisting of H and (C₁-C₆)alkyl;
   or alternatively, R^{6e} and R^{6f} can together form a 5 or 6 membered heterocycle;
   R^{6h} is selected from the group consisting of (C₁-C₆)alkyl, NR^{6k}R^{6m}, and NR^{6m}C(═NR^{6p})NR^{6q}R^{6r};
      R^{6k} and R^{6m} are independently selected from the group consisting of H and (C₁-C₆)alkyl;
      R⁶ⁿ and R^{6p} are independently selected from the group consisting of H and CH₃;
      R^{6q} and R^{6r} are independently selected from the group consisting of H and (C₁-C₆)alkyl;
R^{6b} is selected from the group consisting of H and (C₁-C₆)alkyl;
R^{6c} is selected from the group consisting of H and CH₃;
n is selected from the group consisting of 1, 2, 3, and 4;
R^{6d} is selected from the group consisting of NR^{6s}C(═NR^{6t})NR^{6u}R^{6v}, OH, and NR^{6w}R^{6x};
   R^{6s} and R^{6t} are independently selected from the group consisting of H and CH₃;
   R^{6u}, R^{6v}, R^{6w}, and R^{6x} are independently selected from the group consisting of H and (C₁-C₆)alkyl; and
q is selected from the group consisting of 2, 3, and 4;
Xaa7 is an amino acid residue, wherein the amino acid residue is preferably selected from the group consisting of Formula (VIII), Formula (IX), Formula (X), Formula (XI), Dtc, and Oic:

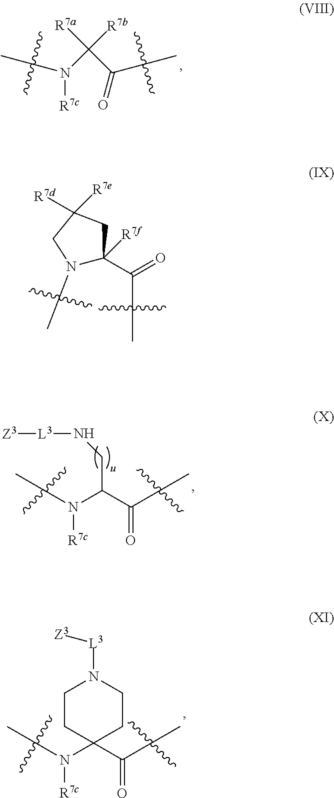

wherein R^{7a} is selected from the group consisting of H, (C₁-C₆)alkyl, and (CH₂)ₜR^{7g};
   R^{7g} is selected from the group consisting of OH, CO₂H, and NR^{7h}R⁷ⁱ;
      R^{7h} and R⁷ⁱ are independently selected from the group consisting of H and (C₁-C₆)alkyl;
   t is selected from the group consisting of 1, 2, 3 and 4;
R^{7b} is selected from the group consisting of H and (C₁-C₆)alkyl;
or alternatively, R^{7a} and R^{7b} can together form a 5 or 6 membered carbocycle or heterocycle,
R^{7c} is selected from the group consisting of H and CH₃;
R^{7d} is selected from the group consisting of H, F, and OH;
R^{7e} is selected from the group consisting of H and F;
R^{7f} is selected from the group consisting of H and CH₃;
u is selected from the group consisting of 2, 3, and 4;
L³ is selected from the group consisting of bond and -(Xab1)ᵥ-,
   wherein v is selected from the group consisting of 1, 2, and 3,
   Xab1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI),
   if v=1, Xab1 is covalently bound to Z³ and covalently bound to a side chain amino function of Formula (X) or of Formula (XI),
   if v=2, a first of the two Xab1 is covalently bound to Z³ and covalently bound to a second of the two Xab1, and the second of the two Xab1 is covalently bound to the first of the two Xab1 and covalently bound to a side chain amino function of Formula (X) or of Formula (XI),
   if v=3, a first of the three Xab1 is covalently bound to Z³ and covalently bound to a second of the three Xab1, the second of the three Xab1 is covalently bound to the first of the three Xab1 and covalently bound to a third of the three Xab1, and the third of the three Xab1 is covalently bound to the second of the three Xab1 and covalently bound to a side chain amino function of Formula (X) or of Formula (XI);
Z³ is selected from the group consisting of H and chelator;
Xaa8 is an α-amino acid residue, wherein the α-nitrogen atom of Xaa8 is unsubstituted or is optionally substituted by CH₃;
Xaa10 is Formula (XII):

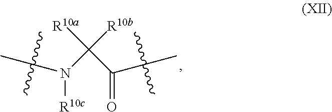

wherein:
R^{10a} is selected from the group consisting of (C₁-C₆)alkyl;
R^{10b} is selected from the group consisting of H and (C₁-C₆)alkyl;
or alternatively, R^{10a} and R^{10b} can together form a 5 or 6 membered carbocycle or heterocycle,
R^{10c} is selected from the group consisting of H and CH₃;
L² is selected from the group consisting of:
   bond and -Xaa11-(Xaa12)ₛ-, such that Xaa11 is covalently bound to Xaa10;
   Xaa11 is an amino acid residue, wherein the amino acid residue is preferably selected from the group consisting of an α-amino acid residue, a β-amino acid residue, a γ-amino acid residue, a δ-amino acid residue, an ε-amino acid residue, an ω-amino acid residue, and an amino acid residue of Formula (YI);
   s is selected from the group consisting of 0, 1, 2, 3, 4, and 5; and
   Xaa12 is each and individually an amino acid residue, wherein the amino acid residue is preferably selected from the group comprising an α-amino acid residue, a β-amino acid residue, a γ-amino acid residue, a δ-amino acid residue, an ε-amino acid residue, an ω-amino acid residue, and an amino acid residue of Formula (YI),
   if s=0, Xaa11 is covalently bound to Z²,
   if s=1, Xaa12 is covalently bound to Xaa11 and covalently bound to Z²,
   if s=2, a first of the two Xaa12 is covalently bound to Xaa11 and covalently bound to a second of the two Xaa12, and the second of the two Xaa12 is covalently bound to the first of the two Xaa12 and covalently bound to Z²,
   if s=3, a first of the three Xaa12 is covalently bound to Xaa11 and covalently bound to a second of the three Xaa12, the second of the three Xaa12 is covalently bound to the first of the three Xaa12 and covalently bound to a third of the three Xaa12, and the third of the three Xaa12 is covalently bound to the second of the three Xaa12 and covalently bound to Z²,
   if s=4, a first of the four Xaa12 is covalently bound to Xaa11 and covalently bound to a second of the four Xaa12, the second of the four Xaa12 is covalently bound to the first of the four Xaa12 and covalently bound to a third of the four Xaa12, the third of the four Xaa12 is covalently bound to the second of the four Xaa12 and covalently bound to a fourth of the four Xaa12, and the fourth of the four Xaa12 is covalently bound to the third of the four Xaa12 and covalently bound to Z²,
   if s=5, a first of the five Xaa12 is covalently bound to Xaa11 and covalently bound to a second of the five Xaa12, the second of the five Xaa12 is covalently bound to the first of the five Xaa12 and covalently bound to a third of the five Xaa12, the third of the five Xaa12 is covalently bound to the second of the five Xaa12 and covalently bound to a fourth of the five Xaa12, the fourth of the five Xaa12 is covalently bound to the third of the five Xaa12 and covalently bound to a fifth of the five Xaa12, and the fifth of the five Xaa12 is covalently bound to the fourth of the five Xaa12 and covalently bound to Z²;
Z² is selected from the group consisting of CT, XDa-chelator and an α-amino acid residue of Formula (CT-I):

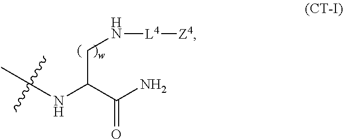

CT is selected from the group consisting of Formula (CT-II), Formula (CT-III), AF488N3K-NH₂, OH, and Throl-OH:

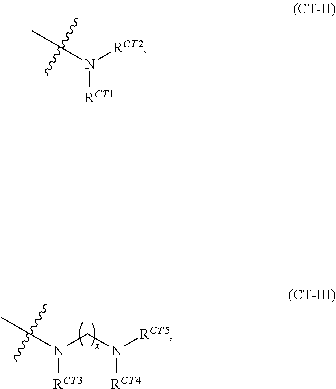

wherein:
   R^{CT1} is selected from the group consisting of H and CH₃;
   R^{CT2} is selected from the group consisting of H and (C₁-C₆)alkyl;
   R^{CT3} and R^{CT4} are each and individually selected from the group consisting of H and CH₃;
   R^{CT5} is selected from the group consisting of H and (C₁-C₆)alkyl;
   x is selected from the group consisting of 2-10;
XDa is a diamine, wherein said diamine is preferably selected from the group consisting of en and Ape;
w is selected from the group consisting of 1, 2, 3, 4, 5, and 6;
L⁴ is selected from the group consisting of a bond and -(Xac1)_{y}-,
wherein:
   y is selected from the group consisting of 0, 1, 2, and 3,
   Xac1 is each and individually an amino acid residue, preferably the amino acid residue is selected from the group comprising an α-amino acid residue, a β-amino acid residue, a γ-amino acid residue, a δ-amino acid residue, an ε-amino acid residue, an ω-amino acid residue and an amino acid residue of Formula (YI),
   if y=0, the side chain amino function of the α-amino acid residue of Formula (CT-I) is covalently bound to Z⁴,
   if y=1, Xac1 is covalently bound to the side chain amino function of the α-amino acid residue of Formula (CT-I) and covalently bound to Z⁴,
   if y=2, a first of the two Xac1 is covalently bound to the side chain amino function of the α-amino acid residue of Formula (CT-I) and covalently bound to a second of the two Xac1, and the second of the two Xac1 is covalently bound to the first of the two Xac1 and covalently bound to Z⁴,
   if y=3, a first of the three Xac1 is covalently bound to the side chain amino function of the α-amino acid residue of Formula (CT-I) and covalently bound to a second of the three Xac1, the second of the three Xac1 is covalently bound to the first of the three Xac1 and covalently bound to a third of the three Xac1, and the third of the three Xac1 is covalently bound to the second of the three Xac1 and covalently bound to Z⁴,
   if y=4, a first of the four Xac1 is covalently bound to the side chain amino function of the α-amino acid residue of Formula (CT-I) and covalently bound to a second of the four Xac1, the second of the four Xac1 is covalently bound to the first of the four Xac1 and covalently bound to a third of the four Xac1, the third of the four Xac1 is covalently bound to the second of the four Xac1 and covalently bound to a fourth of the four Xac1, and the fourth of the four Xac1 is covalently bound to the third of the four Xac1 and covalently bound to Z⁴;
      Z⁴ is selected from the group consisting of H and chelator;
with the proviso that if L² is bond and CT is Formula (CT-II) wherein R^{CT1} is selected from the group consisting of H and CH₃ and R^{CT2} is (C₄-C₆)alkyl, then Xaa10 can be absent; and wherein the compound of Formula (I) may optionally comprise a therapeutically active nuclide or a diagnostically active nuclide.

### BRIEF DESCRIPTION OF THE DRAWINGS

The following detailed description, given by way of example, but not intended to limit the invention, is further illustrated by reference to the following figures from which further features, embodiments and advantages, may be taken.

FIG. 1(a) is an illustration of the PSMA activity inhibition assay.

FIG. 1(b) shows enzyme inhibition curves for PSM-0374, PSM-0516, PSM-0194, PSM-0416, PSM-0424, and 2-PMPA in a human PSMA activity inhibition assay.

FIG. 2 shows a representative radiochromatogram for PSM-0433 labeled with ¹¹¹In.

FIGS. 3(a)-3(ii) show % ID/g uptake (biodistribution) of ¹¹¹In-labeled compounds in a PC3-PIP tumor model in mice (see Example 35).

FIG. 3(a) shows the % ID/g uptake of ¹¹¹In-PSM-0234 at 1 h, 4 h, and 24 h, post injection.

FIG. 3(b) shows the % ID/g uptake of ¹¹¹In-PSM-0425 at 1 h, 4 h, and 24 h, post injection.

FIG. 3(c) shows the % ID/g uptake of ¹¹¹In-PSM-0218 at 1 h, 4 h, and 24 h, post injection.

FIG. 3(d) shows the % ID/g uptake of ¹¹¹In-PSM-0365 at 1 h, 4 h, and 24 h, post injection.

FIG. 3(e) shows the % ID/g uptake of ¹¹¹In-PSM-0580 at 1 h, 4 h, and 24 h, post injection.

FIG. 3(f) shows the % ID/g uptake of ¹¹¹In-PSM-0492 at 1 h, 4 h, and 24 h, post injection.

FIG. 3(g) shows the % ID/g uptake of ¹¹¹In-PSM-0285 at 1 h, 4 h, and 24 h, post injection.

FIG. 3(h) shows the % ID/g uptake of ¹¹¹In-PSM-0237 at 1 h, 4 h, and 24 h, post injection.

FIG. 3(i) shows the % ID/g uptake of ¹¹¹In-PSM-0428 at 1 h, 4 h, and 24 h, post injection.

FIG. 3(j) shows the % ID/g uptake of ¹¹¹In-PSM-0283 at 1 h, 4 h, 24 h, 48 h, and 72 h, post injection.

FIG. 3(k) shows the % ID/g uptake of ¹¹¹In-PSM-0573 at 1 h, 4 h, 24 h, 48 h, and 72 h, post injection.

FIG. 3(l) shows the % ID/g uptake of ¹¹¹In-PSM-0190 at 4 h and 24 h, post injection.

FIG. 3(m) shows the % ID/g uptake of ¹¹¹In-PSM-0239 at 1 h, 4 h, and 24 h, post injection.

FIG. 3(n) shows the % ID/g uptake of ¹¹¹In-PSM-0371 at 1 h, 4 h, and 24 h, post injection.

FIG. 3(o) shows the % ID/g uptake of ¹¹¹In-PSM-0339 at 1 h, 4 h, and 24 h, post injection.

FIG. 3(p) shows the % ID/g uptake of ¹¹¹In-PSM-0301 at 1 h, 4 h, and 24 h, post injection.

FIG. 3(q) shows the % ID/g uptake of ¹¹¹In-PSM-0243 at 1 h, 4 h, and 24 h, post injection.

FIG. 3(r) shows the % ID/g uptake of ¹¹¹In-PSM-0199 at 1 h, 4 h, 24 h, and 48 h, post injection.

FIG. 3(s) shows the % ID/g uptake of ¹¹¹In-PSM-0361 at 1 h, 4 h, 24 h, and 48 h, post injection.

FIG. 3(t) shows the % ID/g uptake of ¹¹¹In-PSM-0273 at 1 h, 4 h, 24 h, and 48 h, post injection.

FIG. 3(u) shows the % ID/g uptake of ¹¹¹In-PSM-0433 at 1 h, 4 h, 24 h, 48 h, and 72 h, post injection.

FIG. 3(v) shows the % ID/g uptake of ¹¹¹In-PSM-0534 at 1 h, 4 h, 24 h, 48 h, and 72 h, post injection.

FIG. 3(w) shows the % ID/g uptake of ¹¹¹In-PSM-0269 at 1 h, 4 h, 24 h, 48 h, and 72 h, post injection.

FIG. 3(x) shows the % ID/g uptake of ¹¹¹In-PSM-0267 at 1 h, 4 h, 24 h, 48 h, and 72 h, post injection.

FIG. 3(y) shows the % ID/g uptake of ¹¹¹In-PSM-0481 at 1 h, 4 h, 24 h, and 48 h, post injection.

FIG. 3(z) shows the % ID/g uptake of ¹¹¹In-PSM-0416 at 1 h, 4 h, 24 h, 48 h, and 72 h, post injection.

FIG. 3(aa) shows the % ID/g uptake of ¹¹¹In-PSM-0194 at 1 h, 4 h, 24 h, and 48 h, post injection.

FIG. 3(bb) shows the % ID/g uptake of ¹¹¹In-PSM-0377 at 1 h, 4 h, 24 h, and 48 h, post injection.

FIG. 3(cc) shows the % ID/g uptake of ¹¹¹In-PSM-0516 at 1 h, 4 h, 24 h, 48 h, and 72 h, post injection.

FIG. 3(dd) shows the % ID/g uptake of ¹¹¹In-PSM-0467 at 1 h, 4 h, 24 h, 48 h, and 72 h, post injection.

FIG. 3(ee) shows the % ID/g uptake of ¹¹¹In-PSM-0384 at 1 h, 4 h, 24 h, 48 h, and 72 h, post injection.

FIG. 3(ff) shows the % ID/g uptake of ¹¹¹In-PSM-0449 at 1 h, 4 h, 24 h, and 48 h, post injection.

FIG. 3(gg) shows the % ID/g uptake of ¹¹¹In-PSM-0241 at 1 h, 4 h, 24 h, 48 h, and 72 h, post injection.

FIG. 3(hh) shows the % ID/g uptake of ¹¹¹In-PSM-0579 at 1 h, 4 h, 24 h, and 48 h, post injection.

FIG. 3(ii) shows the % ID/g uptake of ¹¹¹In-PSM-0531 at 1 h, 4 h, 24 h, 48 h, and 72 h, post injection.

FIGS. 4(a)-4(r) show % ID/g uptake (biodistribution) of ¹¹¹In-labeled compounds in a C₄₋₂ tumor model in mice (see Example 35).

FIG. 4(a) shows the % ID/g uptake of ¹¹¹In-PSM-0285 at 1 h, 4 h, and 24 h post injection.

FIG. 4(b) shows the % ID/g uptake of ¹¹¹In-PSM-0428 at 1 h, 4 h, and 24 h post injection.

FIG. 4(c) shows the % ID/g uptake of ¹¹¹In-PSM-0492 at 1 h, 4 h, 24 h, and 72 h post injection.

FIG. 4(d) shows the % ID/g uptake of ¹¹¹In-PSM-0365 at 1 h, 4 h, and 24 h post injection.

FIG. 4(e) shows the % ID/g uptake of ¹¹¹In-PSM-0218 at 1 h, 4 h, and 24 h post injection.

FIG. 4(f) shows the % ID/g uptake of ¹¹¹In-PSM-0243 at 1 h, 4 h, and 24 h post injection.

FIG. 4(g) shows the % ID/g uptake of ¹¹¹In-PSM-0339 at 1 h, 4 h, and 24 h post injection.

FIG. 4(h) shows the % ID/g uptake of ¹¹¹In-PSM-0301 at 1 h, 4 h, and 24 h post injection.

FIG. 4(i) shows the % ID/g uptake of ¹¹¹In-PSM-0283 at 1 h, 4 h, 24 h, and 72 h post injection.

FIG. 4(j) shows the % ID/g uptake of ¹¹¹In-PSM-0433 at 1 h, 4 h, 24 h, and 72 h post injection.

FIG. 4(k) shows the % ID/g uptake of ¹¹¹In-PSM-0194 at 1 h, 4 h, 24 h, and 72 h post injection.

FIG. 4(l) shows the % ID/g uptake of ¹¹¹In-PSM-0345 at 1 h, 4 h, 24 h, 48 h, and 72 h post injection.

FIG. 4(m) shows the % ID/g uptake of ¹¹¹In-PSM-0380 at 1 h, 4 h, 24 h, 48 h, and 72 h post injection.

FIG. 4(n) shows the % ID/g uptake of ¹¹¹In-PSM-0483 at 1 h, 4 h, 24 h, and 48 h post injection.

FIG. 4(o) shows the % ID/g uptake of ¹¹¹In-PSM-0420 at 1 h, 4 h, 24 h, 48 h, and 72 h post injection.

FIG. 4(p) shows the % ID/g uptake of ¹¹¹In-PSM-0246 at 1 h, 4 h, 24 h, and 48 h post injection.

FIG. 4(q) shows the % ID/g uptake of ¹¹¹In-PSM-0244 at 1 h, 4 h, 24 h, 48 h, and 72 h post injection.

FIG. 4(r) shows the % ID/g uptake of ¹¹¹In-PSM-0203 at 1 h, 4 h, 24 h, and 48 h post injection.

FIG. 5 shows the observed in vivo biodistribution of ¹⁷⁷Lu-PSM-0194 over time (at 4, 24 and 76 hours p.i.) % ID/g, decay-corrected mean values for each organ (blood, kidney (L), kidney (R), liver, tail and tumor), ROI are shown) in the ST1273 model.

FIG. 6 shows individual ST1273 tumor volumes over time after treatment with ¹⁷⁷Lu-PSM-0194 (dotted line at study day 0 indicates the day of treatment).

FIG. 7 shows the amino acid sequence of PSMA (SEQ ID NO: 1).

### DETAILED DESCRIPTION OF THE INVENTION

The present disclosure relates to novel compounds suitable for use as diagnostic agents and/or pharmaceutical agents, for the diagnosis and/or treatment of prostate cancer and other diseases and conditions mediated by PSMA. The present disclosure provides novel compounds, capable of interacting with PSMA, that can deliver a therapeutically active nuclide or a diagnostically active nuclide, which can provide for the detection, treatment, and/or management of various diseases associated with one or more PSMA expressing tumors or cells, including prostate cancer.

The present disclosure is based on the surprising finding that the compounds of the disclosure provide for highly specific and potent binding to PSMA. These compounds are able to interact with PSMA to achieve improved binding affinity and other properties as described herein. The compounds of the invention are surprisingly useful as imaging agents and useful in delivering radionuclides to tumors.

The compounds of the disclosure have one or more advantageous properties, including but not limited to, rapid tumor uptake, prolonged tumor retention, rapid clearance of the compound from non-tumor tissues, improved efficacy, and/or favorable biodistribution properties, with improved toxicity and side effect profiles. In an embodiment and as preferably used herein, a compound shows rapid tumor uptake if, within one hour after administration of the compound to a subject with a tumor, at least 0.1% of the amount of the compound administered to the subject is taken up by the tumor; such tumor uptake is preferably determined by nuclear imaging.

For effective clinical utilization, PSMA ligand selection should be based, for example, on rapid uptake and persistent localization at the target site, with negligible retention in non-targeted tissues. Low levels of endogenous PSMA expression have also been found in organs such as normal prostate, proximal tubules of the kidneys, the lacrimal and salivary glands, the spleen, the liver, the intestinal membranes, the testes, the ovaries, and the brain (Chakravarty, et al 2018 Am J Nucl Med Mol Imaging, 8(4): 247-267), which insofar constitute non-target tissues.

For diagnosis and/or treatment, compounds of the disclosure can be complexed with radionuclides that are α-emitters, β-emitters, γ-emitters, or auger emitters. Radionuclides that are α-emitters are capable of destroying tumors while causing very limited damage to the surrounding healthy tissue due to the short penetration depth of a particles. Their high linear energy transfer (LET) gives them an increased relative biological effectiveness (RBE) as compared to other radionuclide therapies. Furthermore, when α-emitting radionuclides are targeted to specific tumor cells in the body, they can be very effective in destroying metastases, which are difficult to treat by currently employed techniques (de Kruijff et al, 2015 Pharmaceuticals, 8, 321-336). However, toxicity is a primary limitation of the use of α-emitters.

Irradiation of salivary glands is reported to be the main dose-limiting side effect for small molecule PSMA-targeted agents used for the delivery of α-emitting radionuclides such as actinium-225 (²²⁵Ac), particularly due to the irreversible nature of the xerostomia.

Compounds of the invention surprisingly demonstrate low binding to human salivary glands as compared to known PSMA inhibitors.

In the clinic, treatment with an α-emitting radionuclide, such as ²²⁵Ac, complexed to a PSMA inhibitor has been shown to result in irreversible, grade 3/4 xerostomia leading to a significant impairment of the patients' quality of life and thus represents a dose-limiting side effect for therapeutic use of α-emitting small molecule PSMA inhibitors (Tonnesmann et al, 2019, Pharmaceuticals 12, 18). PSMA-targeting antibodies, however, have shown no significant uptake in salivary glands. While the salivary glands are known to possess low levels of PSMA, the detected salivary gland uptake of PSMA-inhibitors in clinical studies does not correlate with the relatively low physiological PSMA-expression in that tissue, meaning the binding to the salivary gland is largely non-specific (Tonnesmann et al, 2019).

The PSMA ligands disclosed herein are surprisingly suitable as carriers for α-emitters for therapy because they can provide for effective treatment of diseases associated with one or more PSMA expressing tumors or cells, including prostate cancer, with reduced salivary gland uptake. This makes it possible to administer such compounds in higher doses, potentially resulting in improved response rates and better tumor control.

The compounds of the disclosure have a favorable uptake ratio of tumor to non-tumor tissue (e.g., salivary glands, kidneys, or other non-tumor tissues).

In certain embodiments, the favorable tumor to non-tumor tissue uptake of the present compounds allows delivery of a radioactive nuclide at a dose that could reduce tumor growth, or partially or completely destroy the tumor, while minimizing side effects. In certain embodiments, due to their favorable uptake ratio of tumor to non-tumor targets, compounds of the present disclosure surprisingly are able to overcome the unwanted side effect of severe xerostomia associated with known PSMA-inhibitors. In certain embodiments, compounds of the present disclosure can advantageously provide for the effective treatment of diseases associated with one or more PSMA expressing tumors or cells, including prostate cancer, and may allow administration of higher doses, potentially resulting in improved response rates and better tumor control. In certain embodiments, compounds of the present disclosure can advantageously maximize therapeutic efficacy while minimizing negative side effects.

In some embodiments, compounds of the disclosure may advantageously be used in a method for the identification of a subject or a method for the selection of a subject from a group of subjects or the method for the stratification of a group of subjects, wherein the subject is likely to respond or likely not to respond to a treatment of a disease, wherein the method comprises carrying out a method of diagnosis using compounds according to the disclosure. In certain embodiments, such methods may advantageously optimize drug treatment, including minimizing risks and maximizing efficacy, for example by helping healthcare professionals identify subjects who might benefit the most from a given therapy and avoid unnecessary treatments.

The present disclosure is further described herein, including in the embodiments below.

Embodiment 1. A compound of Formula (I), or a pharmaceutically acceptable salt, a pharmaceutically acceptable solvate or a hydrate thereof,

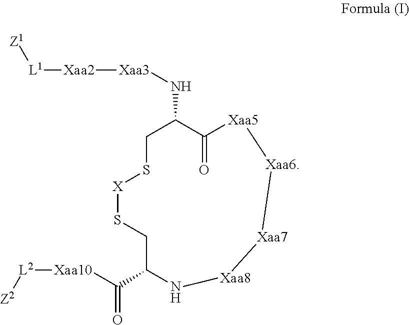

wherein
X is selected from the group consisting of bond and —CH₂—;
Z¹ is selected from the group consisting of chelator and NT;
   NT is selected from the group consisting of H, Ac, Hex, HPA, HO-Succinyl, SaPr, Iva, HYDAc, Bio, nBuCAyl, AF488Ahx, and Hib;
L¹ is selected from the group consisting of a bond and -(Xaa1)ₖ-,
   k is selected from the group consisting of 1, 2, and 3,
   Xaa1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI),

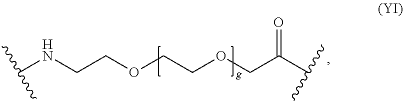

g is an integer selected from the group consisting of 0-23,
wherein,
if k=1, Xaa1 is covalently bound to Z¹ and covalently bound to Xaa2,
if k=2, a first of the two Xaa1 is covalently bound to Z¹ and covalently bound to a second of the two Xaa1, and the second of the two Xaa1 is covalently bound to the first of the two Xaa1 and covalently bound to Xaa2,
if k=3, a first of the three Xaa1 is covalently bound to Z¹ and covalently bound to a second of the three Xaa1, the second of the three Xaa1 is covalently bound to the first of the three Xaa1 and covalently bound to a third of the three Xaa1, and the third of the three Xaa1 is covalently bound to the second of the three Xaa1 and covalently bound to Xaa2;
wherein when L¹ is bond, then Z¹ is NT;
Xaa2 is selected from the group consisting of Formula (II) and Formula (III):

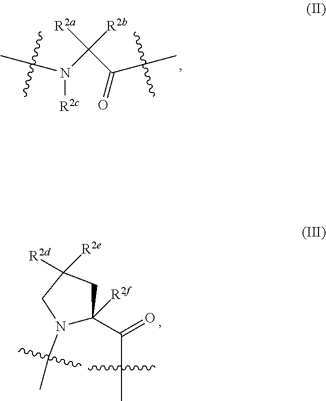

wherein R^{2a} is selected from the group consisting of H, (C₁-C₆)alkyl, and CH₂R^{2g};
   R^{2g} is selected from the group consisting of OH, and CO₂H;
R^{2b} is selected from the group consisting of H and (C₁-C₆)alkyl;
or alternatively, R^{2a} and R^{2b} can together form a 5 or 6 membered carbocycle or heterocycle,
R^{2c} is selected from the group consisting of H and CH₃;
R^{2d} is selected from the group consisting of H, F, and OH;
R^{2e} is selected from the group consisting of H and F;
R^{2f} is selected from the group consisting of H and CH₃;
with the proviso that Xaa2 can be absent when L¹ is bond and NT is Hib;
Xaa3 is selected from Formula (TV):

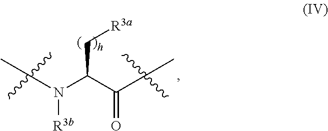

wherein R^{3a} is selected from the group consisting of aryl, (C₅-C₆)heteroaryl, indol-3-yl, 6-chloro-1H-indol-3-yl, and —S—CH₂-phenyl; and wherein said aryl or said heteroaryl ring of R^{3a} is optionally substituted by 1, 2, or 3 substituents independently selected from the group consisting of halogen, (C₁-C₆)alkyl, CN, OH, and —O(C₁-C₃)alkyl, wherein said (C₁-C₆)alkyl may optionally be substituted by one or more fluorine;
R^{3b} is selected from the group consisting of H and CH₃;
h is selected from the group consisting of 1 and 2;
Xaa5 is selected from the group consisting of Formula (Va), Formula (Vb), Hgn, lys, Nle, Tap, Aph, and Gln:

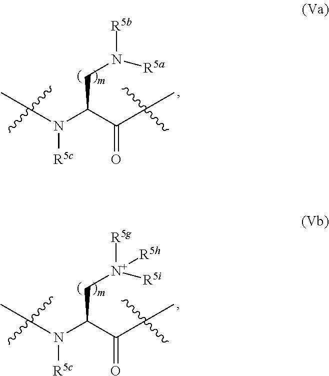

wherein R^{5a} is selected from the group consisting of H, (C₁-C₆)alkyl, Ac, C(═NR^{5d})NR^{5e}R^{5f}, and Bio;
   R^{5d} is selected from the group consisting of H and CH₃;
   R^{5e} and R^{5f} are independently selected from the group consisting of H and (C₁-C₆)alkyl;
R^{5b} is selected from the group consisting of H and (C₁-C₆)alkyl;
R^{5c} is selected from the group consisting of H and CH₃;
m is selected from the group consisting of 2, 3, 4, and 5; and
R^{5g}, R^{5h}, and R⁵ⁱ are independently selected from the group consisting of (C₁-C₆)alkyl;
Xaa6 is selected from the group consisting of Formula (VI), Formula (VII), Nle, and arg:

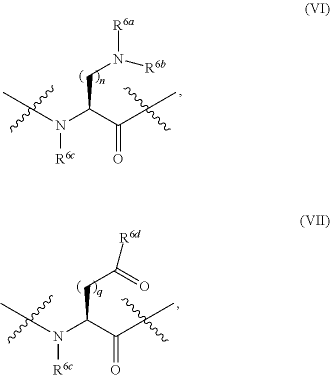

wherein R^{6a} is selected from the group consisting of H, C(═NR^{6e})NR^{6f}R^{6g}, C(═O)R^{6h} and pyridyl;
   R^{6e} is selected from the group consisting of H and CH₃;
   R^{6f} is selected from the group consisting of H, (C₁-C₆)alkyl, Ac, NO₂, and C(═O)NR⁶ⁱR^{6j};
      R⁶ⁱ and R^{6j} are independently selected from the group consisting of H and (C₁-C₂)alkyl;
   R^{6g} is selected from the group consisting of H and (C₁-C₆)alkyl;
   or alternatively, R^{6e} and R^{6f} can together form a 5 or 6 membered heterocycle;
   R^{6h} is selected from the group consisting of (C₁-C₆)alkyl, NR^{6k}R^{6m}, and NR⁶ⁿC(═NR^{6p})NR^{6q}R^{6r};
      R^{6k} and R^{6m} are independently selected from the group consisting of H and (C₁-C₆)alkyl;
      R⁶ⁿ and R^{6p} are independently selected from the group consisting of H and CH₃;
      R^{6q} and R^{6r} are independently selected from the group consisting of H and (C₁-C₆)alkyl;
R^{6b} is selected from the group consisting of H and (C₁-C₆)alkyl;
R^{6c} is selected from the group consisting of H and CH₃;
n is selected from the group consisting of 1, 2, 3, and 4;
R^{6d} is selected from the group consisting of NR^{6s}C(═NR^{6t})NR^{6u}R^{6v}, OH, and NR^{6w}R^{6x};
   R^{6s} and R^{6t} are independently selected from the group consisting of H and CH₃;
   R^{6u}, R^{6v}, R^{6w}, and R^{6x} are independently selected from the group consisting of H and (C₁-C₆)alkyl; and
q is selected from the group consisting of 2, 3, and 4;
Xaa7 is an amino acid residue, wherein the amino acid residue is preferably selected from the group consisting of Formula (VIII), Formula (IX), Formula (X), Formula (XI), Dtc, and Oic:

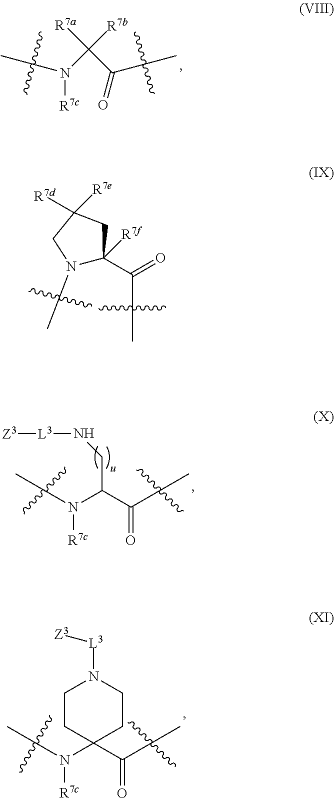

wherein R^{7a} is selected from the group consisting of H, (C₁-C₆)alkyl, and (CH₂)ₜR^{7g};
   R^{7g} is selected from the group consisting of OH, CO₂H, and NR^{7h}R⁷ⁱ;
      R^{7h} and R⁷ⁱ are independently selected from the group consisting of H and (C₁-C₆)alkyl;
   t is selected from the group consisting of 1, 2, 3 and 4;
R^{7b} is selected from the group consisting of H and (C₁-C₆)alkyl;
or alternatively, R^{7a} and R^{7b} can together form a 5 or 6 membered carbocycle or heterocycle,
R^{7c} is selected from the group consisting of H and CH₃;
R^{7d} is selected from the group consisting of H, F, and OH;
R^{7e} is selected from the group consisting of H and F;
R^{7f} is selected from the group consisting of H and CH₃;
u is selected from the group consisting of 2, 3, and 4;
L³ is selected from the group consisting of bond and -(Xab1)ᵥ-,
   wherein v is selected from the group consisting of 1, 2, and 3,
   Xab1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI),
   if v=1, Xab1 is covalently bound to Z³ and covalently bound to a side chain amino function of Formula (X) or of Formula (XI),
   if v=2, a first of the two Xab1 is covalently bound to Z³ and covalently bound to a second of the two Xab1, and the second of the two Xab1 is covalently bound to the first of the two Xab1 and covalently bound to a side chain amino function of Formula (X) or of Formula (XI),
   if v=3, a first of the three Xab1 is covalently bound to Z³ and covalently bound to a second of the three Xab1, the second of the three Xab1 is covalently bound to the first of the three Xab1 and covalently bound to a third of the three Xab1, and the third of the three Xab1 is covalently bound to the second of the three Xab1 and covalently bound to a side chain amino function of Formula (X) or of Formula (XI);
Z³ is selected from the group consisting of H and chelator;
Xaa8 is an α-amino acid residue, wherein the α-nitrogen atom of Xaa8 is unsubstituted or is optionally substituted by CH₃;
Xaa10 is Formula (XII):

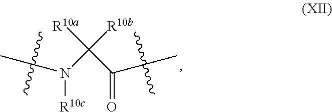

wherein:
R^{10a} is selected from the group consisting of (C₁-C₆)alkyl;
R^{10b} is selected from the group consisting of H and (C₁-C₆)alkyl;
or alternatively, R^{10a} and R^{10b} can together form a 5 or 6 membered carbocycle or heterocycle,
R^{10c} is selected from the group consisting of H and CH₃;
L² is selected from the group consisting of:
   bond and -Xaa11-(Xaa12)ₛ-, such that Xaa11 is covalently bound to Xaa10;
   Xaa11 is an amino acid residue, wherein the amino acid residue is preferably selected from the group consisting of an α-amino acid residue, a β-amino acid residue, a γ-amino acid residue, a δ-amino acid residue, an ε-amino acid residue, an ω-amino acid residue, and an amino acid residue of Formula (YI);
   s is selected from the group consisting of 0, 1, 2, 3, 4, and 5; and
   Xaa12 is each and individually an amino acid residue, wherein the amino acid residue is preferably selected from the group comprising an α-amino acid residue, a β-amino acid residue, a γ-amino acid residue, a δ-amino acid residue, an ε-amino acid residue, an ω-amino acid residue, and an amino acid residue of Formula (YI),
   if s=0, Xaa11 is covalently bound to Z²,
   if s=1, Xaa12 is covalently bound to Xaa11 and covalently bound to Z²,
   if s=2, a first of the two Xaa12 is covalently bound to Xaa11 and covalently bound to a second of the two Xaa12, and the second of the two Xaa12 is covalently bound to the first of the two Xaa12 and covalently bound to Z²,
   if s=3, a first of the three Xaa12 is covalently bound to Xaa11 and covalently bound to a second of the three Xaa12, the second of the three Xaa12 is covalently bound to the first of the three Xaa12 and covalently bound to a third of the three Xaa12, and the third of the three Xaa12 is covalently bound to the second of the three Xaa12 and covalently bound to Z²,
   if s=4, a first of the four Xaa12 is covalently bound to Xaa11 and covalently bound to a second of the four Xaa12, the second of the four Xaa12 is covalently bound to the first of the four Xaa12 and covalently bound to a third of the four Xaa12, the third of the four Xaa12 is covalently bound to the second of the four Xaa12 and covalently bound to a fourth of the four Xaa12, and the fourth of the four Xaa12 is covalently bound to the third of the four Xaa12 and covalently bound to Z²,
   if s=5, a first of the five Xaa12 is covalently bound to Xaa11 and covalently bound to a second of the five Xaa12, the second of the five Xaa12 is covalently bound to the first of the five Xaa12 and covalently bound to a third of the five Xaa12, the third of the five Xaa12 is covalently bound to the second of the five Xaa12 and covalently bound to a fourth of the five Xaa12, the fourth of the five Xaa12 is covalently bound to the third of the five Xaa12 and covalently bound to a fifth of the five Xaa12, and the fifth of the five Xaa12 is covalently bound to the fourth of the five Xaa12 and covalently bound to Z².
Z² is selected from the group consisting of CT, XDa-chelator and an α-amino acid residue of Formula (CT-I):

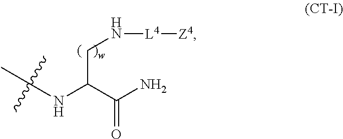

CT is selected from the group consisting of Formula (CT-II), Formula (CT-III), AF488N3K-NH₂, OH, and Throl-OH:

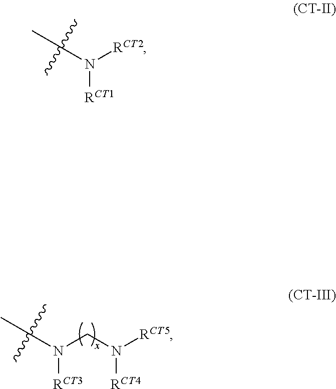

wherein:
   R^{CT1} is selected from the group consisting of H and CH₃;
   R^{CT2} is selected from the group consisting of H and (C₁-C₆)alkyl;
   R^{CT3} and R^{CT4} are each and individually selected from the group consisting of H and CH₃;
   R^{CT5} is selected from the group consisting of H and (C₁-C₆)alkyl;
   x is selected from the group consisting of 2-10;
XDa is a diamine, wherein said diamine is preferably selected from the group consisting of en and Ape;
w is selected from the group consisting of 1, 2, 3, 4, 5, and 6;
L⁴ is selected from the group consisting of a bond and -(Xac1)_{y}-,
wherein:
   y is selected from the group consisting of 0, 1, 2, and 3,
   Xac1 is each and individually an amino acid residue, preferably the amino acid residue is selected from the group comprising an α-amino acid residue, a β-amino acid residue, a γ-amino acid residue, a δ-amino acid residue, an F-amino acid residue, an ω-amino acid residue and an amino acid residue of Formula (YI),
   if y=0, the side chain amino function of the α-amino acid residue of Formula (CT-I) is covalently bound to Z⁴,
   if y=1, Xac1 is covalently bound to the side chain amino function of the α-amino acid residue of Formula (CT-I) and covalently bound to Z⁴,
   if y=2, a first of the two Xac1 is covalently bound to the side chain amino function of the α-amino acid residue of Formula (CT-I) and covalently bound to a second of the two Xac1, and the second of the two Xac1 is covalently bound to the first of the two Xac1 and covalently bound to Z⁴,
   if y=3, a first of the three Xac1 is covalently bound to the side chain amino function of the α-amino acid residue of Formula (CT-I) and covalently bound to a second of the three Xac1, the second of the three Xac1 is covalently bound to the first of the three Xac1 and covalently bound to a third of the three Xac1, and the third of the three Xac1 is covalently bound to the second of the three Xac1 and covalently bound to Z⁴,
   if y=4, a first of the four Xac1 is covalently bound to the side chain amino function of the α-amino acid residue of Formula (CT-I) and covalently bound to a second of the four Xac1, the second of the four Xac1 is covalently bound to the first of the four Xac1 and covalently bound to a third of the four Xac1, the third of the four Xac1 is covalently bound to the second of the four Xac1 and covalently bound to a fourth of the four Xac1, and the fourth of the four Xac1 is covalently bound to the third of the four Xac1 and covalently bound to Z⁴;
Z⁴ is selected from the group consisting of H and chelator;
with the proviso that if L² is bond and CT is Formula (CT-II) wherein R^{CT1} is selected from the group consisting of H and CH₃ and R^{CT2} is (C₄-C₆)alkyl, then Xaa10 can be absent;
and wherein the compound of Formula (I) may optionally comprise a therapeutically active nuclide or a diagnostically active nuclide.

Embodiment 2. A compound of Formula (I), or a pharmaceutically acceptable salt, a pharmaceutically acceptable solvate or a hydrate thereof,

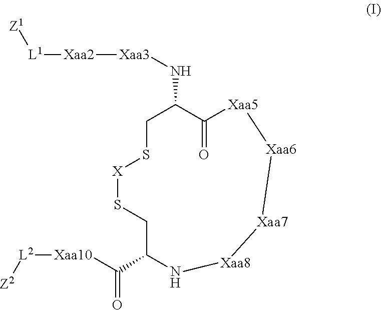

wherein
X is selected from the group consisting of bond and —CH₂—;
Z¹ is selected from the group consisting of chelator and NT;
   NT is selected from the group consisting of H, Ac, Hex, HPA, HO-Succinyl, SaPr, Iva, HYDAc, Bio, nBuCAyl, AF488Ahx, and Hib;
L¹ is selected from the group consisting of bond and -(Xaa1)ₖ-;
   k is selected from the group consisting of 1, 2, and 3;
   wherein,
   if k=1, Xaa1 is covalently bound to Z¹ and covalently bound to Xaa2,
   if k=2, a first of the two Xaa1 is covalently bound to Z1 and covalently bound to a second of the two Xaa1, and the second of the two Xaa1 is covalently bound to the first of the two Xaa1 and covalently bound to Xaa2,
   if k=3, a first of the three Xaa1 is covalently bound to Z1 and covalently bound to a second of the three Xaa1, the second of the three Xaa1 is covalently bound to the first of the three Xaa1 and covalently bound to a third of the three Xaa1, and the third of the three Xaa1 is covalently bound to the second of the three Xaa1 and covalently bound to Xaa2;
   wherein when L¹ is bond, then Z¹ is NT;
   Xaa1 is each and individually selected from the group consisting of Thr, Ala, Ser, Pamp, Leu, Ile, Nmt, Pamb, Ahx, APAc, PPAc, Bal, Cmp, Pab, O2Oc, Met, and Ttds;
Xaa2 is selected from the group consisting of Aib, Ala, Amd, Ams, amd, ams, Deg, Nmg, Pam, and Pro; with the proviso that Xaa2 can be absent when L¹ is bond and NT is Hib;
Xaa3 is selected from the group consisting of Phe, Nmf, 1Ni, 2Ni, 6Clw, Cys(Bzl), Hfe, Trp, Mpa, Opa, and Ppa, wherein Phe, Nmf, and Hfe are optionally substituted by 1 or 2 substituents independently selected from the group consisting of halogen, CH₃, CN, CF₃, and OH;
Xaa5 is selected from the group consisting of Formula (Va), Formula (Vb), Hgn, lys, Nle, Tap, Aph, and Gln:

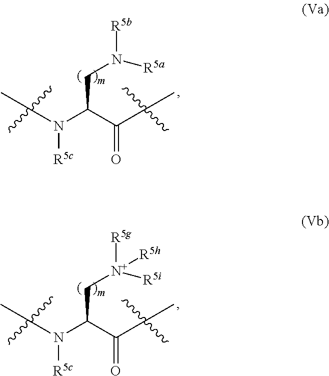

wherein R^{5a} is selected from the group consisting of H, (C₁-C₆)alkyl, Ac, C(═NR^{5d})NR^{5e}R^{5f} and Bio;
   R^{5d} is selected from the group consisting of H and CH₃;
   R^{5e} and R^{5f} are independently selected from the group consisting of H and (C₁-C₆)alkyl;
R^{5b} is selected from the group consisting of H and (C₁-C₆)alkyl;
R^{5c} is selected from the group consisting of H and CH₃;
m is selected from the group consisting of 2, 3, 4, and 5; and
R^{5g}, R^{5h}, and R⁵ⁱ are independently selected from the group consisting of (C₁-C₆)alkyl;
Xaa6 is selected from the group consisting of Formula (VI), Formula (VII), Nle, and arg:

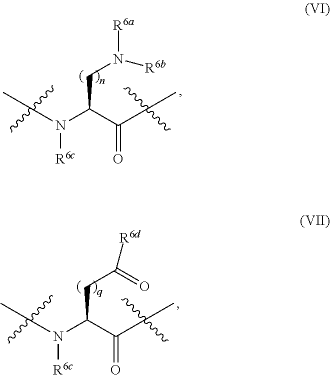

wherein R^{6a} is selected from the group consisting of H, C(═NR^{6e})NR^{6f}R^{6g}, C(═O)R^{6h}, and pyridyl;
   R^{6e} is selected from the group consisting of H and CH₃;
   R^{6f} is selected from the group consisting of H, (C₁-C₆)alkyl, Ac, NO₂, and C(═O)NR⁶ⁱR^{6j};
      R⁶ⁱ and R^{6j} are independently selected from the group consisting of H and (C₁-C₂)alkyl;
   R^{6g} is selected from the group consisting of H and (C₁-C₆)alkyl;
   or alternatively, R^{6e} and R^{6f} can together form a 5 or 6 membered heterocycle;
   R^{6h} is selected from the group consisting of (C₁-C₆)alkyl, NR^{6k}R^{6m}, and NR⁶ⁿC(═NR^{6p})NR^{6q}R^{6r};
      R^{6k} and R^{6m} are independently selected from the group consisting of H and (C₁-C₆)alkyl;
      R^{6m} and R^{6p} are independently selected from the group consisting of H and CH₃;
      R^{6q} and R^{6r} are independently selected from the group consisting of H and (C₁-C₆)alkyl;
R^{6b} is selected from the group consisting of H and (C₁-C₆)alkyl;
R^{6c} is selected from the group consisting of H and CH₃;
n is selected from the group consisting of 1, 2, 3, and 4;
R^{6d} is selected from the group consisting of NR^{6s}C(═NR^{6t})NR^{6u}R^{6v}, OH, and NR^{6w}R^{6x};
   R^{6s} and R^{6t} are independently selected from the group consisting of H and CH₃;
   R^{6u}, R^{6v}, R^{6w}, and R^{6x} are independently selected from the group consisting of H and (C₁-C₆)alkyl; and
q is selected from the group consisting of 2, 3, and 4;
Xaa7 is selected from the group consisting of Formula (VIII), Formula (IX), Formula (X), Formula (XI), Dtc, and Oic:

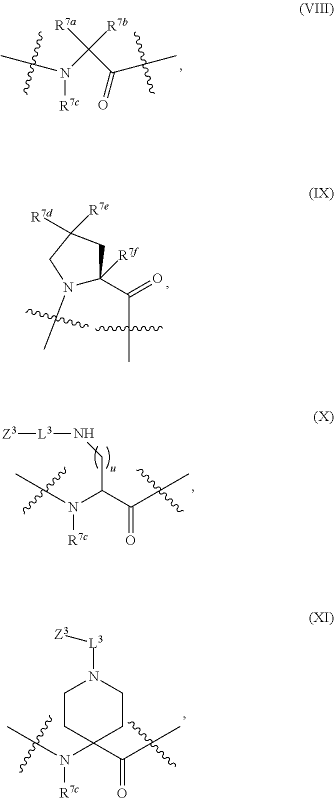

wherein R^{7a} is selected from the group consisting of H, (C₁-C₆)alkyl, and (CH₂)ₜR^{7g};
   R^{7g} is selected from the group consisting of OH, CO₂H, and NR^{7h}R⁷ⁱ;
      R^{7h} and R⁷ⁱ are independently selected from the group consisting of H and (C₁-C₆)alkyl;
   t is selected from the group consisting of 1, 2, 3 and 4;
R^{7b} is selected from the group consisting of H and (C₁-C₆)alkyl;
or alternatively, R^{7a} and R^{7b} can together form a 5 or 6 membered carbocycle or heterocycle,
R^{7c} is selected from the group consisting of H and CH₃;
R^{7d} is selected from the group consisting of H, F, and OH;
R^{7e} is selected from the group consisting of H and F;
R^{7f} is selected from the group consisting of H and CH₃;
u is selected from the group consisting of 2, 3, and 4;
L³ is selected from the group consisting of bond and -(Xab1)ᵥ-;
   v is selected from the group consisting of 1, 2, and 3;
   Xab1 is each and individually selected from the group consisting of Ttds, Pamb, APAc, O2Oc, Ahx, Pab, and Cmp;
   wherein, if v=1, Xab1 is covalently bound to Z³ and covalently bound to a side chain amino function of Formula (X) or of Formula (XI),
   if v=2, a first of the two Xab1 is covalently bound to Z³ and covalently bound to a second of the two Xab1, and the second of the two Xab1 is covalently bound to the first of the two Xab1 and covalently bound to a side chain amino function of Formula (X) or of Formula (XI),
   if v=3, a first of the three Xab1 is covalently bound to Z3 and covalently bound to a second of the three Xab1, the second of the three Xab1 is covalently bound to the first of the three Xab1 and covalently bound to a third of the three Xab1, and the third of the three Xab1 is covalently bound to the second of the three Xab1 and covalently bound to a side chain amino function of Formula (X) or of Formula (XI);
Z³ is selected from the group consisting of H and chelator;
Xaa8 is an α-amino acid residue, wherein the α-nitrogen atom of Xaa8 is unsubstituted or is optionally substituted by CH₃;
Xaa10 is Formula (XII)

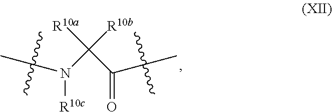

wherein
R^{10a} is selected from the group consisting of (C₁-C₆)alkyl;
R^{10b} is selected from the group consisting of H and CH₃;
R^{10c} is selected from the group consisting of H and CH₃;
L² is selected from the group consisting of
   bond and -Xaa11-(Xaa12)ₛ-, such that Xaa11 is covalently bound to Xaa10;
   Xaa11 is selected from the group consisting of Thr, Ala, Bal, Gab, Gln, Glu, Gly, Leu, Nmt, Phe, Pro, and Trp;
   s is selected from the group consisting of 0, 1, 2, 3, 4, and 5; and
   Xaa12 is each and individually selected from the group consisting of Asp, asp, Ala, Gab, Ttds, Pamb, Cmp, O2Oc, APAc, Gly, Ser, Lys(Bio), and Pab;
   wherein, if s=0, Xaa11 is covalently bound to Z²,
   if s=1, Xaa12 is covalently bound to Xaa11 and covalently bound to Z²,
   if s=2, a first of the two Xaa12 is covalently bound to Xaa11 and covalently bound to a second of the two Xaa12, and the second of the two Xaa12 is covalently bound to the first of the two Xaa12 and covalently bound to Z²,
   if s=3, a first of the three Xaa12 is covalently bound to Xaa11 and covalently bound to a second of the three Xaa12, the second of the three Xaa12 is covalently bound to the first of the three Xaa12 and covalently bound to a third of the three Xaa12, and the third of the three Xaa12 is covalently bound to the second of the three Xaa12 and covalently bound to Z²,
   if s=4, a first of the four Xaa12 is covalently bound to Xaa11 and covalently bound to a second of the four Xaa12, the second of the four Xaa12 is covalently bound to the first of the four Xaa12 and covalently bound to a third of the four Xaa12, the third of the four Xaa12 is covalently bound to the second of the four Xaa12 and covalently bound to a fourth of the four Xaa12, and the fourth of the four Xaa12 is covalently bound to the third of the four Xaa12 and covalently bound to Z²,
   if s=5, a first of the five Xaa12 is covalently bound to Xaa11 and covalently bound to a second of the five Xaa12, the second of the five Xaa12 is covalently bound to the first of the five Xaa12 and covalently bound to a third of the five Xaa12, the third of the five Xaa12 is covalently bound to the second of the five Xaa12 and covalently bound to a fourth of the five Xaa12, the fourth of the five Xaa12 is covalently bound to the third of the five Xaa12 and covalently bound to a fifth of the five Xaa12, and the fifth of the five Xaa12 is covalently bound to the fourth of the five Xaa12 and covalently bound to Z²;
Z² is selected from the group consisting of CT, -en-chelator, -Ape-chelator, and Formula (CT-I)

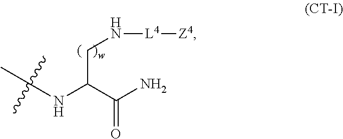

CT is selected from the group consisting of NH₂, en, en(Me)₂, en(Me), NHBu, NHnPen, AF488N3K-NH₂, OH, and Throl-OH;
w is selected from the group consisting of 2, 3, and 4;
L⁴ is selected from the group consisting of a bond, Ttds, Pamb, APAc, O2Oc, Ahx, Pab, and Cmp;
Z⁴ is chelator;
with the proviso that Xaa10 can be absent if L² is bond and CT is NHnPen;
and wherein the compound of Formula (I) may optionally comprise a therapeutically active nuclide or a diagnostically active nuclide.

Embodiment 3. The compound of any one of Embodiments 1 and 2, or a pharmaceutically acceptable salt, a pharmaceutically acceptable solvate or a hydrate thereof, wherein only 1 of Z¹, Z², Z³, and Z⁴ comprises a chelator.

Embodiment 4. The compound of any one of Embodiments 1, 2, and 3, or a pharmaceutically acceptable salt, a pharmaceutically acceptable solvate or a hydrate thereof, wherein X is a bond.

Embodiment 5. The compound of any one or Embodiments 1, 2, and 3, or a pharmaceutically acceptable salt, a pharmaceutically acceptable solvate or a hydrate thereof, wherein X is CH₂.

Embodiment 6. The compound, or a pharmaceutically acceptable salt, a pharmaceutically acceptable solvate or a hydrate thereof, of any one of Embodiments 1, 2, 3, 4, and 5, wherein
Z² is CT; and
Xaa7 is selected from the group consisting of Formula (VIII), Formula (IX), Dtc, and Oic.

Embodiment 7. The compound of Embodiment 6, or a pharmaceutically acceptable salt, a pharmaceutically acceptable solvate or a hydrate thereof wherein Z¹ is chelator.

Embodiment 8. The compound of any one of Embodiments 6 and 7, or a pharmaceutically acceptable salt, a pharmaceutically acceptable solvate or a hydrate thereof, wherein
L¹ is -(Xaa1)ₖ-; and wherein k is selected from the group consisting of 1 and 2.

Embodiment 9. The compound of any one of Embodiments 6, 7, and 8, or a pharmaceutically acceptable salt, a pharmaceutically acceptable solvate or a hydrate thereof, wherein k is 2, and L¹ is Formula (XIII):

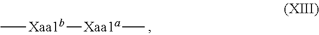

wherein Xaa1^{a} is covalently bound to Xaa2;
Xaa1^{a} is selected from the group consisting of Thr, Ile, and Leu; and
Xaa1^{b} is selected from the group consisting of Cmp, Ttds, Pamb, Ahx, APAc, Bal, O2Oc, and Pab.

Embodiment 10. The compound of Embodiment 9, or a pharmaceutically acceptable salt, a pharmaceutically acceptable solvate or a hydrate thereof, wherein Xaa1^{a} is Thr.

Embodiment 11. The compound of any one of Embodiments 9 and 10, or a pharmaceutically acceptable salt, a pharmaceutically acceptable solvate or a hydrate thereof, wherein Xaa1^{b} is Cmp.

Embodiment 12. The compound of any one of Embodiments 6, 7, and 8, or a pharmaceutically acceptable salt, a pharmaceutically acceptable solvate or a hydrate thereof, wherein k is 1, and Xaa1 is selected from the group consisting of Cmp, Pamb, Bal, Pab, Ahx, APAc, Thr, Pamp, and PPAc.

Embodiment 13. The compound of Embodiment 12, or a pharmaceutically acceptable salt, a pharmaceutically acceptable solvate or a hydrate thereof, wherein Xaa1 is Pamb.

Embodiment 14. The compound of any one of Embodiments 6, 7, 8, 9, 10, 11, 12 and 13, or a pharmaceutically acceptable salt, a pharmaceutically acceptable solvate or a hydrate thereof, wherein L² is selected from the group consisting of bond and -Xaa11-(Xaa12)ₛ-; and s is selected from the group consisting of 0 and 1.

Embodiment 15. The compound of Embodiment 14, or a pharmaceutically acceptable salt, a pharmaceutically acceptable solvate or a hydrate thereof, wherein L² is -Xaa11-(Xaa12)ₛ-;
s is selected from the group consisting of 0 and 1; and
Xaa11 is selected from the group consisting of Thr, Bal, Gln, Phe, Gab, Nmt, Gly, Leu, Trp, Glu, and Pro.

Embodiment 16. The compound of any one of Embodiments 14 and 15, or a pharmaceutically acceptable salt, a pharmaceutically acceptable solvate or a hydrate thereof, wherein Xaa11 is selected from the group consisting of Thr and Bal.

Embodiment 17. The compound of Embodiment 16, or a pharmaceutically acceptable salt, a pharmaceutically acceptable solvate or a hydrate thereof, wherein Xaa11 is Thr.

Embodiment 18. The compound of Embodiment 16, or a pharmaceutically acceptable salt, a pharmaceutically acceptable solvate or a hydrate thereof, wherein Xaa11 is Bal.

Embodiment 19. The compound of any one of Embodiments 15, 16, 17 and 18, or a pharmaceutically acceptable salt, a pharmaceutically acceptable solvate or a hydrate thereof, wherein s is 0.

Embodiment 20. The compound of any one of Embodiments 14, 15, 16, 17 and 18, or a pharmaceutically acceptable salt, a pharmaceutically acceptable solvate or a hydrate thereof, wherein s is 1; and Xaa12 is Asp.

Embodiment 21. The compound of Embodiment 14, or a pharmaceutically acceptable salt, a pharmaceutically acceptable solvate or a hydrate thereof, wherein L² is bond.

Embodiment 22. The compound of any one of Embodiments 14, 15, 16, 17, 18, 19, 20 and 21, or a pharmaceutically acceptable salt, a pharmaceutically acceptable solvate or a hydrate thereof, wherein CT is selected from the group consisting of NH₂, en, en(Me), en(Me)₂, NHBu, Throl-OH, and OH.

Embodiment 23. The compound of Embodiment 22, or a pharmaceutically acceptable salt, a pharmaceutically acceptable solvate or a hydrate thereof, wherein CT is NH₂.

Embodiment 24. The compound of any one of Embodiments 6, 7, 8, 9, 10, 11, 12, 13 and 14, or a pharmaceutically acceptable salt, a pharmaceutically acceptable solvate or a hydrate thereof, wherein L²-Z² is selected from the group consisting of Thr-NH₂, Bal-NH₂, Glu-NH₂, Pro-NH₂, Gln-NH₂, Trp-NH₂, Leu-NH₂, Gly-NH₂, Nmt-NH₂, Gab-NH₂, Phe-NH₂, Throl-OH, and Thr-OH.

Embodiment 25. The compound of Embodiment 24, or a pharmaceutically acceptable salt, a pharmaceutically acceptable solvate or a hydrate thereof, wherein L²-Z² is selected from the group consisting of Thr-NH₂ and Bal-NH₂.

Embodiment 26. The compound of Embodiment 25, or a pharmaceutically acceptable salt, a pharmaceutically acceptable solvate or a hydrate thereof, wherein L²-Z² is Thr-NH₂.

Embodiment 27. The compound of Embodiment 25, or a pharmaceutically acceptable salt, a pharmaceutically acceptable solvate or a hydrate thereof, wherein L²-Z² is Bal-NH₂.

Embodiment 28. The compound of any one of Embodiments 6, 7, 8, 9, 10, 11, 12, 13, 14, 15, 16, 17, 18, 19, 20, 21, 22, 23, 24, 25, 26 and 27, or a pharmaceutically acceptable salt, a pharmaceutically acceptable solvate or a hydrate thereof, wherein Xaa7 is selected from the group consisting of Formula (VIII), Formula (IX), Dtc, and Oic, wherein
R^{7a} is selected from the group consisting of H, (C₁-C₆)alkyl, and (CH₂)ₜR^{7g};
   t is selected from the group consisting of 1 and 2; and
   R^{7g} is selected from the group consisting of OH, CO₂H, and NH₂.

Embodiment 29. The compound of Embodiment 28, or a pharmaceutically acceptable salt, a pharmaceutically acceptable solvate or a hydrate thereof, wherein Xaa7 is selected from the group consisting of Aib, Ala, Glu, Pro, Dfp, glu, Amd, 4Tfp, Pam, Deg, Nmg, Ams, ams, amd, Dtc, and Oic.

Embodiment 30. The compound of Embodiment 28, or a pharmaceutically acceptable salt, a pharmaceutically acceptable solvate or a hydrate thereof, wherein Xaa7 is selected from Formula (VIII), and wherein
R^{7a} is selected from the group consisting of H, (C₁-C₂)alkyl, CH₂OH, CH₂CO₂H, and CH₂CH₂CO₂H;
   R^{7b} is selected from the group consisting of H and (C₁-C₂)alkyl; and
   R^{7c} is H.

Embodiment 31. The compound of any one of Embodiments 28, 29 and 30, or a pharmaceutically acceptable salt, a pharmaceutically acceptable solvate or a hydrate thereof, wherein Xaa7 is selected from the group consisting of Aib and Ala.

Embodiment 32. The compound of any one of Embodiments 28, 29, 30 and 31, or a pharmaceutically acceptable salt, a pharmaceutically acceptable solvate or a hydrate thereof, wherein Xaa7 is Aib.

Embodiment 33. The compound of any one of Embodiments 28, 29, 30 and 31, or a pharmaceutically acceptable salt, a pharmaceutically acceptable solvate or a hydrate thereof, wherein Xaa7 is Ala.

Embodiment 34. A compound, or a pharmaceutically acceptable salt, a pharmaceutically acceptable solvate or a hydrate thereof, of any one of Embodiments 1, 2, 3, 4 and 5, wherein
Z¹ is NT, and
Xaa7 is selected from the group consisting of Formula (VIII), Formula (IX), Dtc, and Oic.

Embodiment 35. A compound of Embodiment 34, or a pharmaceutically acceptable salt, a pharmaceutically acceptable solvate or a hydrate thereof, wherein Z² is selected from the group consisting of en-chelator, -Ape-chelator, and Formula (CT-I), wherein if Z² is Formula (CT-I), then Z⁴ is chelator.

Embodiment 36. A compound of any one of Embodiments 34 and 35, or a pharmaceutically acceptable salt, a pharmaceutically acceptable solvate or a hydrate thereof, wherein
L¹ is selected from the group consisting of bond and (Xaa1)ₖ; and
k is selected from the group consisting of 1 and 2.

Embodiment 37. The compound of Embodiment 36, or a pharmaceutically acceptable salt, a pharmaceutically acceptable solvate or a hydrate thereof, wherein k is 2 and L¹ is Formula (XIII)

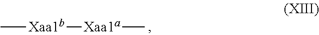

wherein Xaa1^{a} is covalently bound to Xaa2 of Formula (I);
Xaa1^{a} is Thr; and
Xaa1^{b} is Met or Cmp.

Embodiment 38. The compound of Embodiment 37, or a pharmaceutically acceptable salt, a pharmaceutically acceptable solvate or a hydrate thereof, wherein Xaa1^{b} is Met.

Embodiment 39. The compound of any one of Embodiments 37 and 38, or a pharmaceutically acceptable salt, a pharmaceutically acceptable solvate or a hydrate thereof, wherein NT is selected from the group consisting of H, Ac, and nBuCAyl.

Embodiment 40. The compound of Embodiment 39, or a pharmaceutically acceptable salt, a pharmaceutically acceptable solvate or a hydrate thereof, wherein NT is Ac.

Embodiment 41. The compound of Embodiment 36, or a pharmaceutically acceptable salt, a pharmaceutically acceptable solvate or a hydrate thereof, wherein k is 1, and Xaa1 is selected from the group consisting of Thr, Ala, Pamp, and Ser.

Embodiment 42. The compound of any one of Embodiments 37 and 38, or a pharmaceutically acceptable salt, a pharmaceutically acceptable solvate or a hydrate thereof, wherein Xaa1 is Thr.

Embodiment 43. The compound of any one of Embodiments 41 and 42, or a pharmaceutically acceptable salt, a pharmaceutically acceptable solvate or a hydrate thereof, wherein Xaa1 is selected from the group consisting of Thr and Pamp; and NT is selected from the group consisting of Ac, nBuCAyl, and Hex.

Embodiment 44. The compound of Embodiment 43, or a pharmaceutically acceptable salt, a pharmaceutically acceptable solvate or a hydrate thereof, wherein Xaa1 is Thr and NT is Ac.

Embodiment 45. The compound of Embodiment 36, or a pharmaceutically acceptable salt, a pharmaceutically acceptable solvate or a hydrate thereof, wherein L¹ is bond.

Embodiment 46. The compound of Embodiment 45, or a pharmaceutically acceptable salt, a pharmaceutically acceptable solvate or a hydrate thereof, wherein NT is selected from the group consisting of Ac, HPA, HYDAc, Iva, SaPr, and HO-Succinyl.

Embodiment 47. The compound of Embodiment 46, or a pharmaceutically acceptable salt, a pharmaceutically acceptable solvate or a hydrate thereof, wherein NT is Ac.

Embodiment 48. The compound of any one of Embodiments 34, 35, 36, 37, 38, 39, 40, 41, 42, 43, 44, 45, 46 and 47, or a pharmaceutically acceptable salt, a pharmaceutically acceptable solvate or a hydrate thereof, wherein
L² is -Xaa11-(Xaa12)ₛ-; and
s is selected from the group consisting of 0, 1, and 2.

Embodiment 49. The compound of Embodiment 48, or a pharmaceutically acceptable salt, a pharmaceutically acceptable solvate or a hydrate thereof, wherein Z² is Formula (CT-I).

Embodiment 50. The compound of any one of Embodiments 48 and 49, or a pharmaceutically acceptable salt, a pharmaceutically acceptable solvate or a hydrate thereof, wherein Xaa11 is Thr.

Embodiment 51. The compound of any one of Embodiments 48, 49 and 50, or a pharmaceutically acceptable salt, a pharmaceutically acceptable solvate or a hydrate thereof, wherein L² is
-Xaa11-(Xaa12)ₛ; s is 1; and
Xaa12 is selected from the group consisting of Asp, Cmp, Ttds, Pamb, O2Oc, APAc, and Pab.

Embodiment 52. The compound of Embodiment 51, or a pharmaceutically acceptable salt, a pharmaceutically acceptable solvate or a hydrate thereof, wherein Xaa11 is Thr and Xaa12 is Cmp.

Embodiment 53. The compound of any one of Embodiments 48, 49 and 50, or a pharmaceutically acceptable salt, a pharmaceutically acceptable solvate or a hydrate thereof, wherein L² is -Xaa11-(Xaa12)ₛ; and s is 2; and L² has the structure
-Xaa11-Xaa12^{a}-Xaa12^{b}-; wherein
Xaa12^{a} is selected from the group consisting of Asp, Cmp, Ttds, Pamb, O2Oc, APAc, and Pab; and
Xaa12^{b} is Ttds.

Embodiment 54. The compound of Embodiment 53, or a pharmaceutically acceptable salt, a pharmaceutically acceptable solvate or a hydrate thereof, wherein Xaa11 is Thr, Xaa12^{a} is Asp and Xaa12^{b} is Ttds.

Embodiment 55. The compound of any one of Embodiments 34, 35, 36, 37, 38, 39, 40, 41, 42, 43, 44, 45, 46, 47 and 48, or a pharmaceutically acceptable salt, a pharmaceutically acceptable solvate or a hydrate thereof, wherein Z² is selected from the group consisting of -en-chelator and -Ape-chelator.

Embodiment 56. The compound of Embodiment 55, or a pharmaceutically acceptable salt, a pharmaceutically acceptable solvate or a hydrate thereof, wherein Z² is en-chelator.

Embodiment 57. The compound of any one of Embodiments 55 and 56, or a pharmaceutically acceptable salt, a pharmaceutically acceptable solvate or a hydrate thereof, wherein L² is a bond or Xaa11.

Embodiment 58. The compound of Embodiment 57, or a pharmaceutically acceptable salt, a pharmaceutically acceptable solvate or a hydrate thereof, wherein L² is a bond.

Embodiment 59. The compound of Embodiment 57, or a pharmaceutically acceptable salt, a pharmaceutically acceptable solvate or a hydrate thereof, wherein L² is Xaa11 and Xaa11 is Thr.

Embodiment 60. The compound of Embodiment 55, or a pharmaceutically acceptable salt, a pharmaceutically acceptable solvate or a hydrate thereof, wherein Z² is -Ape-chelator, L² is -Xaa11-; and Xaa11 is Thr.

Embodiment 61. The compound of any one of Embodiments 34, 35, 36, 37, 38, 39, 40, 41, 42, 43, 44, 45, 46, 47, 48, 49, 50, 51, 52, 53, 54, 55, 56, 57, 58, 59 and 60, or a pharmaceutically acceptable salt, a pharmaceutically acceptable solvate or a hydrate thereof, wherein Xaa7 is selected from Formula (VIII), Formula (IX), Dtc, and Oic, wherein
R^{7a} is selected from the group consisting of H, (C₁-C₆) alkyl, and (CH₂)ₜR^{7g};
   R^{7g} is selected from the group consisting of OH and CO₂H;
   t is selected from the group consisting of 1 and 2; and
R^{7b} is selected from the group consisting of H and (C₁-C₆) alkyl.

Embodiment 62. The compound of Embodiment 61, or a pharmaceutically acceptable salt, a pharmaceutically acceptable solvate or a hydrate thereof, wherein Xaa7 is selected from the group consisting of Aib, Ala, Glu, Pro, Dfp, glu, Amd, 4Tfp, Pam, Deg, Nmg, Ams, ams, amd, Dtc, and Oic.

Embodiment 63. The compound of Embodiment 61, or a pharmaceutically acceptable salt, a pharmaceutically acceptable solvate or a hydrate thereof, wherein Xaa7 is Formula (VIII), and wherein
R^{7a} is selected from the group consisting of H, (C₁-C₂)alkyl, CH₂OH, CH₂CO₂H, and CH₂CH₂CO₂H;
R^{7b} is selected from the group consisting of H and (C₁-C₂)alkyl; and
R^{7c} is H.

Embodiment 64. The compound of any one of Embodiments 61, 62 and 63, or a pharmaceutically acceptable salt, a pharmaceutically acceptable solvate or a hydrate thereof, wherein Xaa7 is selected from the group consisting of Ala and Aib.

Embodiment 65. The compound of any one of Embodiments 61, 62, 63 and 64, or a pharmaceutically acceptable salt, a pharmaceutically acceptable solvate or a hydrate thereof, wherein Xaa7 is Aib.

Embodiment 66. The compound of any one of Embodiments 49, 50, 51, 52, 53, 54, 55, 56, 57, 58, 59, 60, 61, 62, 63, 64 and 65, or a pharmaceutically acceptable salt, a pharmaceutically acceptable solvate or a hydrate thereof, wherein Xaa7 is Ala.

Embodiment 67. A compound, or a pharmaceutically acceptable salt, a pharmaceutically acceptable solvate or a hydrate thereof, of any one of Embodiments 1, 2, 3, 4 and 5, wherein Xaa7 is selected from the group consisting of Formula (X) and Formula (XI);
Z³ is chelator;
Z¹ is NT; and
Z² is CT.

Embodiment 68. The compound of Embodiment 67, or a pharmaceutically acceptable salt, a pharmaceutically acceptable solvate or a hydrate thereof, wherein
L¹ is selected from the group consisting of bond and -(Xaa1)ₖ-; and k is 1.

Embodiment 69. The compound of Embodiment 68, or a pharmaceutically acceptable salt, a pharmaceutically acceptable solvate or a hydrate thereof, wherein Xaa1 is Thr.

Embodiment 70. The compound of Embodiment 68, or a pharmaceutically acceptable salt, a pharmaceutically acceptable solvate or a hydrate thereof, wherein L¹ is bond.

Embodiment 71. The compound of any one of Embodiments 67, 68, 69 and 70, or a pharmaceutically acceptable salt, a pharmaceutically acceptable solvate or a hydrate thereof, wherein NT is selected from the group consisting of Ac, SaPr, Iva, and HPA.

Embodiment 72. The compound of Embodiment 71, or a pharmaceutically acceptable salt, a pharmaceutically acceptable solvate or a hydrate thereof, wherein NT is Ac.

Embodiment 73. The compound of any one of Embodiments 67, 68, 69, 70, 71 and 72, or a pharmaceutically acceptable salt, a pharmaceutically acceptable solvate or a hydrate thereof, wherein L² is selected from the group consisting of bond and -Xaa11-(Xaa12)ₛ-, wherein s is selected from the group consisting of 0 and 1.

Embodiment 74. The compound of Embodiment 73, or a pharmaceutically acceptable salt, a pharmaceutically acceptable solvate or a hydrate thereof, wherein Xaa11 is selected from the group consisting of Thr, Gln, Phe, Gab, Nmt, Bal, Gly, Leu, Trp, Glu, and Pro.

Embodiment 75. The compound of any one of Embodiments 73 and 74, or a pharmaceutically acceptable salt, a pharmaceutically acceptable solvate or a hydrate thereof, wherein Xaa11 is Thr.

Embodiment 76. The compound of any one of Embodiments 73, 74 and 75, or a pharmaceutically acceptable salt, a pharmaceutically acceptable solvate or a hydrate thereof, wherein s is 1; and Xaa12 is Asp.

Embodiment 77. The compound of any one of Embodiments 73, 74, 75 and 76, or a pharmaceutically acceptable salt, a pharmaceutically acceptable solvate or a hydrate thereof, wherein CT is NH₂.

Embodiment 78. The compound of any one of Embodiments 67, 68, 69, 70, 71, 72, 73, 74, 75, 76 and 77, or a pharmaceutically acceptable salt, a pharmaceutically acceptable solvate or a hydrate thereof, wherein Xaa7 is selected from the group consisting of Lys and Apc, wherein to the ε-nitrogen atom of Lys or the γ-nitrogen atom of Apc a chelator is attached, wherein an optional linker is interspersed between Apc or Lys and the chelator.

Embodiment 79. The compound of Embodiment 78, or a pharmaceutically acceptable salt, a pharmaceutically acceptable solvate or a hydrate thereof, wherein the chelator is selected from the group consisting of DOTA, DOTAGA, LSC, NOPO, PCTA, DOTAM, Macropa, Crown, NOTA, and NODAGA.

Embodiment 80. The compound of any one of Embodiments 78 and 79, or a pharmaceutically acceptable salt, a pharmaceutically acceptable solvate or a hydrate thereof, wherein if a linker is interspersed, the linker is selected from the group consisting of 020c, Pab, Ahx, APAc, Pamb, Cmp and Ttds.

Embodiment 81. The compound of any one of Embodiments 67, 68, 69, 70, 71, 72, 73, 74, 75, 76, 77, 78, 79 and 80, or a pharmaceutically acceptable salt, a pharmaceutically acceptable solvate or a hydrate thereof, wherein Xaa7 is selected from the group consisting of Apc(DOTA), Lys(DOTAGA-O2Oc), Lys(DOTA-O2Oc), Lys(DOTA-Pab), Lys(DOTA-Ahx), Lys(DOTA-APAc), Lys(DOTA-Pamb), Lys(DOTA-Cmp), Lys(DOTA-Ttds), Lys(DOTA).

Embodiment 82. The compound of any one of Embodiments 67, 68, 69, 70, 71, 72, 73, 74, 75, 76 and 77, or a pharmaceutically acceptable salt, a pharmaceutically acceptable solvate or a hydrate thereof, wherein Xaa7 is selected from Formula (X), wherein
u is 4;
L³ is -(Xab1)ᵥ-;
v is 1; and
Xab1 is selected from the group consisting of Ttds, Pamb, APAc, O2Oc, Ahx, and Pab.

Embodiment 83. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81 and 82, or a pharmaceutically acceptable salt, a pharmaceutically acceptable solvate or a hydrate thereof, wherein chelator is selected from the group consisting of DOTA, DOTAGA, LSC, NOPO, PCTA, DOTAM, Macropa, Crown, NOTA, and NODAGA.

Embodiment 84. The compound of Embodiment 83, or a pharmaceutically acceptable salt, a pharmaceutically acceptable solvate or a hydrate thereof, wherein the chelator is selected from the group consisting of DOTA and DOTAGA.

Embodiment 85. The compound of Embodiment 84, or a pharmaceutically acceptable salt, a pharmaceutically acceptable solvate or a hydrate thereof, wherein the chelator is DOTA.

Embodiment 86. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 and 85, or a pharmaceutically acceptable salt, a pharmaceutically acceptable solvate or a hydrate thereof, wherein Xaa2 is selected from the group consisting of Aib, Ala, Ams, ams, Deg, Pam, and Pro.

Embodiment 87. The compound of Embodiment 86, or a pharmaceutically acceptable salt, a pharmaceutically acceptable solvate or a hydrate thereof, wherein Xaa2 is selected from the group consisting of Aib, Ala, Pam, Deg, Ams, and ams.

Embodiment 88. The compound of Embodiment 87, or a pharmaceutically acceptable salt, a pharmaceutically acceptable solvate or a hydrate thereof, wherein Xaa2 is Aib.

Embodiment 89. The compound of Embodiment 87, or a pharmaceutically acceptable salt, a pharmaceutically acceptable solvate or a hydrate thereof, wherein Xaa2 is Ala.

Embodiment 90. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88 and 89, or a pharmaceutically acceptable salt, a pharmaceutically acceptable solvate or a hydrate thereof, wherein Xaa3 is selected from the group consisting of Phe, 1Ni, 2Ni, 6Clw, Cys(Bzl), Hfe, and Trp, wherein Phe, Nmf, and Hfe are optionally substituted by 1 or 2 substituents independently selected from the group consisting of Cl, CH₃, F, CN, CF₃, and OH.

Embodiment 91. The compound of Embodiment 90, or a pharmaceutically acceptable salt, a pharmaceutically acceptable solvate or a hydrate thereof, wherein Xaa3 is selected from the group consisting of Phe, Nmf, and Hfe, and wherein Phe, Nmf, and Hfe are optionally substituted by 1 substituent selected from the group consisting of Cl, CH₃, F, CN, CF₃, and OH.

Embodiment 92. The compound of Embodiment 91, or a pharmaceutically acceptable salt, a pharmaceutically acceptable solvate or a hydrate thereof, wherein Xaa3 is Phe optionally substituted by 1 substituent selected from the group consisting of Cl, CH₃, F, CN, CF₃, and OH.

Embodiment 93. The compound of Embodiment 92, or a pharmaceutically acceptable salt, a pharmaceutically acceptable solvate or a hydrate thereof, wherein Xaa3 is Phe optionally substituted by 1 substituent selected from the group consisting of Cl, CH₃, F, CN, CF₃.

Embodiment 94. The compound of Embodiment 93, or a pharmaceutically acceptable salt, a pharmaceutically acceptable solvate or a hydrate thereof, wherein Xaa3 is selected from the group consisting of Phe, Pcf, Mcf, Mff, Mnf, Mmf, Pmf, Pnf, Pff, Mtf, and Ptf.

Embodiment 95. The compound of Embodiment 94, or a pharmaceutically acceptable salt, a pharmaceutically acceptable solvate or a hydrate thereof, wherein Xaa3 is selected from the group consisting of Phe and Pcf.

Embodiment 96. The compound of Embodiment 94, or a pharmaceutically acceptable salt, a pharmaceutically acceptable solvate or a hydrate thereof, wherein Xaa3 is Phe.

Embodiment 97. The compound of Embodiment 94, or a pharmaceutically acceptable salt, a pharmaceutically acceptable solvate or a hydrate thereof, wherein Xaa3 is Pcf.

Embodiment 98. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 and 97, or a pharmaceutically acceptable salt, a pharmaceutically acceptable solvate or a hydrate thereof, wherein Xaa5 is selected from the group consisting of Formula (Va), Formula (Vb), Hgn, and Nle, and
R^{5g}, R^{5h}, and R⁵ⁱ are CH₃.

Embodiment 99. The compound of Embodiment 98, or a pharmaceutically acceptable salt, a pharmaceutically acceptable solvate or a hydrate thereof, wherein Xaa5 is selected from Formula (Va), and
R^{5a} is selected from the group consisting of H, CH₃, Ac, and C(═NR^{5d})NR^{5e}R^{5f};
   R^{5e} and R^{5f} are independently selected from the group consisting of H and CH₃;
R^{5b} is H;
R^{5c} is H; and
m is selected from the group consisting of 3 and 4.

Embodiment 100. The compound of Embodiment 99, or a pharmaceutically acceptable salt, a pharmaceutically acceptable solvate or a hydrate thereof, wherein R^{5a} is selected from the group consisting of H and CH₃; and m is 4.

Embodiment 101. The compound of any one of Embodiments 98, 99 and 100, or a pharmaceutically acceptable salt, a pharmaceutically acceptable solvate or a hydrate thereof, wherein Xaa5 is Lys.

Embodiment 102. The compound of any one of Embodiments 98, 99 and 100, or a pharmaceutically acceptable salt, a pharmaceutically acceptable solvate or a hydrate thereof, wherein Xaa5 is Lys(Me).

Embodiment 103. The compound of any one of Embodiments 98, 99 and 100, or a pharmaceutically acceptable salt, a pharmaceutically acceptable solvate or a hydrate thereof, wherein Xaa5 is Kip.

Embodiment 104. The compound of any one of Embodiments 98, 99 and 100, or a pharmaceutically acceptable salt, a pharmaceutically acceptable solvate or a hydrate thereof, wherein Xaa5 is KMe3.

Embodiment 105. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103 and 104, or a pharmaceutically acceptable salt, a pharmaceutically acceptable solvate or a hydrate thereof, wherein Xaa6 is selected from the group consisting of Formula (VI), Formula (VII), and Nle,
wherein R^{6a} is selected from the group consisting of H, C(═NR^{6e})NR^{6f}R^{6g}, and C(═O)R^{6h};
   R^{6e} is H;
   R^{6f} is selected from the group consisting of H, CH₃, Ac, NO₂, and C(═O)NR⁶ⁱR^{6j};
   R^{6g} is selected from the group consisting of H and CH₃;
   R^{6h} is selected from the group consisting of CH₃, NH₂, and NHC(═NH)NH₂;
R^{6b} is H;
R^{6c} is H; and
R^{6d} is selected from the group consisting of NHC(═NH)NH₂ and NH₂.

Embodiment 106. The compound of Embodiment 105, or a pharmaceutically acceptable salt, a pharmaceutically acceptable solvate or a hydrate thereof, wherein Xaa6 is selected from the group consisting of Arg, Arg(Me), Cit, Egd, RMe2a, RMe3, Nle, Gln, Lys(Ac), Hgn, Arg(EtCAyl), Urr, Arg(Ac), Gln(Gu), Orn, Har, RMe2, and Eew.

Embodiment 107. The compound of Embodiment 105, or a pharmaceutically acceptable salt, a pharmaceutically acceptable solvate or a hydrate thereof, wherein Xaa6 is selected from Formula (VI), and wherein
n is 3;
R^{6a} is C(═NH)NHR^{6f}; and
   R^{6f} is selected from the group consisting of H, Ac, NO₂, and CH₃.

Embodiment 108. The compound of any one of Embodiments 105, 106 and 107, or a pharmaceutically acceptable salt, a pharmaceutically acceptable solvate or a hydrate thereof, wherein Xaa6 is Arg.

Embodiment 109. The compound of any one of Embodiments 105, 106 and 107, or a pharmaceutically acceptable salt, a pharmaceutically acceptable solvate or a hydrate thereof, wherein Xaa6 is Arg(Me).

Embodiment 110. The compound of any one of Embodiments 105 and 106, or a pharmaceutically acceptable salt, a pharmaceutically acceptable solvate or a hydrate thereof, wherein Xaa6 is Cit.

Embodiment 111. The compound of any one of Embodiments 105 and 106, or a pharmaceutically acceptable salt, a pharmaceutically acceptable solvate or a hydrate thereof, wherein Xaa6 is Egd.

Embodiment 112. The compound of any one of Embodiments 105 and 106, or a pharmaceutically acceptable salt, a pharmaceutically acceptable solvate or a hydrate thereof, wherein Xaa6 is RMe2a.

Embodiment 113. The compound of any one of Embodiments 105 and 106, or a pharmaceutically acceptable salt, a pharmaceutically acceptable solvate or a hydrate thereof, wherein Xaa6 is RMe3.

Embodiment 114. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 and 113, or a pharmaceutically acceptable salt, a pharmaceutically acceptable solvate or a hydrate thereof, wherein Xaa8 is selected from the group consisting of Formula (XIV), Gly, Val, Met, Ile and Thr:

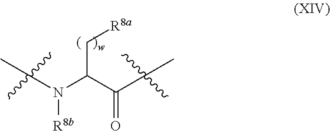

wherein R^{8a}, is selected from the group consisting of H, OH, NH₂, COOH, C(═O)NH₂, NHC(═NH)NH₂, (C₁-C₈)alkyl, aryl, and heteroaryl;
w is selected from the group consisting of 1, 2, and 3; and
R^{8b} is selected from the group consisting of H and CH₃.

Embodiment 115. The compound of Embodiment 114, or a pharmaceutically acceptable salt, a pharmaceutically acceptable solvate or a hydrate thereof, wherein
R^{8a} is selected from the group consisting of OH, COOH, C(═O)NH₂, phenyl, NHC(═NH)NH₂, indole, and CH(CH₃)₂; and
w is selected from the group consisting of 1 and 2.

Embodiment 116. The compound of any one of Embodiments 114 and 115, or a pharmaceutically acceptable salt, a pharmaceutically acceptable solvate or a hydrate thereof, wherein Xaa8 is selected from the group consisting of Asn, Trp, Phe, Arg, Ser, Gly, Leu, Asp, Nmn, Glu, and asn.

Embodiment 117. The compound of Embodiment 116, or a pharmaceutically acceptable salt, a pharmaceutically acceptable solvate or a hydrate thereof, wherein Xaa8 is Asn.

Embodiment 118. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116 and 117, or a pharmaceutically acceptable salt, a pharmaceutically acceptable solvate or a hydrate thereof, wherein Xaa10 is Formula (XII):

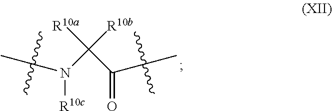

wherein
R^{10b} is selected from the group consisting of H and CH₃;
R^{10c} is H.

Embodiment 119. The compound of Embodiment 118, or a pharmaceutically acceptable salt, a pharmaceutically acceptable solvate or a hydrate thereof, wherein Xaa10 is selected from the group consisting of Tle, Leu, Val, Npg, and Ile.

Embodiment 120. The compound of Embodiment 119, or a pharmaceutically acceptable salt, a pharmaceutically acceptable solvate or a hydrate thereof, wherein Xaa10 is selected from the group consisting of Tle, Leu, Val, and Npg.

Embodiment 121. The compound of Embodiment 120, or a pharmaceutically acceptable salt, a pharmaceutically acceptable solvate or a hydrate thereof, wherein Xaa10 is Tle.

Embodiment 122. The compound of Embodiment 120, or a pharmaceutically acceptable salt, a pharmaceutically acceptable solvate or a hydrate thereof, wherein Xaa10 is Leu.

Embodiment 123. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 and 33, or a pharmaceutically acceptable salt, a pharmaceutically acceptable solvate or a hydrate thereof, wherein the compound is a compound of Formula (Ia).

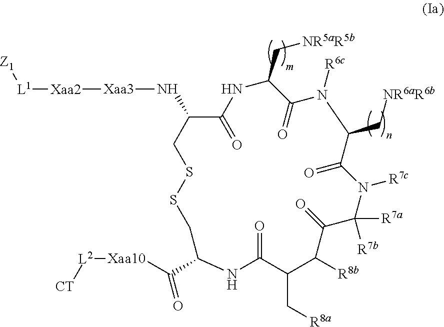

Embodiment 124. The compound of Embodiments 123, or a pharmaceutically acceptable salt, a pharmaceutically acceptable solvate or a hydrate thereof, wherein Xaa3 is selected from the group consisting of Phe, 1Ni, 2Ni, 6Clw, Cys(Bzl), Hfe, and Trp, wherein Phe, Nmf, and Hfe are optionally substituted by 1 or 2 substituents independently selected from the group consisting of Cl, CH₃, F, CN, CF₃, and OH.

Embodiment 125. The compound of Embodiment 124, or a pharmaceutically acceptable salt, a pharmaceutically acceptable solvate or a hydrate thereof, wherein Xaa3 is selected from the group consisting of Phe, Nmf, and Hfe, and wherein Phe, Nmf, and Hfe are optionally substituted by 1 substituent selected from the group consisting of Cl, CH₃, F, CN, CF₃, and OH.

Embodiment 126. The compound of Embodiment 125, or a pharmaceutically acceptable salt, a pharmaceutically acceptable solvate or a hydrate thereof, wherein Xaa3 is Phe optionally substituted by 1 substituent selected from the group consisting of Cl, CH₃, F, CN, CF₃, and OH.

Embodiment 127. The compound of Embodiment 125, or a pharmaceutically acceptable salt, a pharmaceutically acceptable solvate or a hydrate thereof, wherein Xaa3 is Phe.

Embodiment 128. The compound of Embodiment 125, or a pharmaceutically acceptable salt, a pharmaceutically acceptable solvate or a hydrate thereof, wherein Xaa3 is Pcf.

Embodiment 129. The compound of any one of Embodiments 123, 124, 125, 126, 127 and 128, or a pharmaceutically acceptable salt, a pharmaceutically acceptable solvate or a hydrate thereof, wherein Xaa10 is Formula (XII), wherein

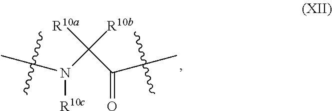

R^{10b} is selected from the group consisting of H and CH₃;
R^{10c} is H.

Embodiment 130. The compound of Embodiment 129, or a pharmaceutically acceptable salt, a pharmaceutically acceptable solvate or a hydrate thereof, wherein Xaa10 is selected from the group consisting of a compound of Formula (XIV)

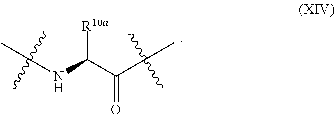

Embodiment 131. The compound of any one of Embodiments 129 and 130, or a pharmaceutically acceptable salt, a pharmaceutically acceptable solvate or a hydrate thereof, wherein R^{10a} is selected from the group consisting of C(CH₃)₃, CH₂CH(CH₃)₂, CH(CH₃)₂, CH(CH₃)C₂H₅ and CH₂C(CH₃)₃.

Embodiment 132. The compound of Embodiment 131, or a pharmaceutically acceptable salt, a pharmaceutically acceptable solvate or a hydrate thereof, wherein R^{10a} is C(CH₃)₃.

Embodiment 133. The compound of Embodiment 131, or a pharmaceutically acceptable salt, a pharmaceutically acceptable solvate or a hydrate thereof, wherein R^{10a} is CH₂CH(CH₃)₂.

Embodiment 134. The compound of any one of Embodiments 123, 124, 125, 126, 127, 128, 129, 130, 131, 132 and 133, or a pharmaceutically acceptable salt, a pharmaceutically acceptable solvate or a hydrate thereof, wherein the compound is a compound of Formula (Ib)

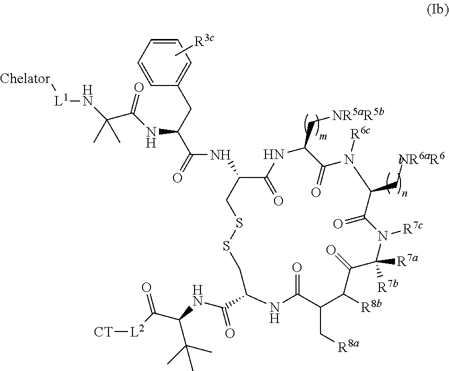

wherein R^{3c} is selected from the group consisting of H, Cl, CH₃, F, CN, CF₃, and OH; and
R^{3c} is at the meta or para position of the phenyl ring of Formula (Ib).

Embodiment 135. The compound of Embodiment 134, or a pharmaceutically acceptable salt, a pharmaceutically acceptable solvate or a hydrate thereof, wherein R^{3c} is H.

Embodiment 136. The compound of Embodiment 134, or a pharmaceutically acceptable salt, a pharmaceutically acceptable solvate or a hydrate thereof, wherein R^{3c} is Cl, wherein R^{3c} is at the paraposition of the phenyl ring of Formula (Ib).

Embodiment 137. The compound of any one of Embodiments 123, 124, 125, 126, 127, 128, 129, 130, 131, 132, 133 and 134, or a pharmaceutically acceptable salt, a pharmaceutically acceptable solvate or a hydrate thereof, wherein
R^{5a} is selected from the group consisting of H, CH₃, C(CH₃)₂, Ac, and C(═NR^{5d})NR^{5e}R^{5f};
   R^{5d}, R^{5e} and R^{5f} are independently selected from the group consisting of H and CH₃;
R^{5b} is selected from the group consisting of H and CH₃; and
m is selected from the group consisting of 3 and 4.

Embodiment 138. The compound of Embodiment 137, or a pharmaceutically acceptable salt, a pharmaceutically acceptable solvate or a hydrate thereof, wherein R^{5a} is selected from the group consisting of H and CH₃, R^{5b} is H and m is 4.

Embodiment 139. The compound of Embodiment 137, or a pharmaceutically acceptable salt, a pharmaceutically acceptable solvate or a hydrate thereof, wherein R^{5a} is CH₃, R^{5b} is H and m is 4.

Embodiment 140. The compound of Embodiment 137, or a pharmaceutically acceptable salt, a pharmaceutically acceptable solvate or a hydrate thereof, wherein R^{5a} is C(CH₃)₂, R^{5b} is H and m is 4

Embodiment 141. The compound of any one of Embodiments 123, 124, 125, 126, 127, 128, 129, 130, 131, 132, 133, 134, 135, 136, 137, 138, 139 and 140, or a pharmaceutically acceptable salt, a pharmaceutically acceptable solvate or a hydrate thereof, wherein
R^{6a} is selected from the group consisting of H, C(═NR^{6e})NR^{6f}R^{6g}, and C(═O)R^{6h};
   R^{6e} is selected from the group consisting of H and CH₃,
   R^{6f} is selected from the group consisting of H, CH₃, Ac, NO₂, and C(═O)NR⁶ⁱR^{6j};
      R⁶ⁱ is selected from the group consisting of H and (C₁-C₂)alkyl;
      R^{6j} is H;
   R^{6g} is selected from the group consisting of H and CH₃;
   R^{6h} is selected from the group consisting of CH₃, NH₂, and NHC(═NH)NH₂;
R^{6b} and R^{6c} are each and individually selected from the group consisting of H and CH₃; and
n is selected from the group consisting of 3 and 4.

Embodiment 142. The compound of Embodiment 141, or a pharmaceutically acceptable salt, a pharmaceutically acceptable solvate or a hydrate thereof, wherein n is selected from the group consisting of 3 and 4, and R^{6c} is H.

Embodiment 143. The compound of any one of Embodiments 123, 124, 125, 126, 127, 128, 129, 130, 131, 132, 133, 134, 135, 136, 137, 138, 139, 140, 141 and 142, or a pharmaceutically acceptable salt, a pharmaceutically acceptable solvate or a hydrate thereof, wherein
n is 3; and
R^{6a} is C(═NR^{6e})NR^{6f}R^{6g};
   R^{6e} is H;
   R^{6f} is selected from the group consisting of H, CH₃, Ac, NO₂, and C(═O)NHR⁶ⁱ;
   R^{6g} is H; and
R^{6b} is H.

Embodiment 144. The compound of Embodiment 143, or a pharmaceutically acceptable salt, a pharmaceutically acceptable solvate or a hydrate thereof, wherein R^{6f} is selected from the group consisting of H, Ac, NO₂, and CH₃.

Embodiment 145. The compound of any one of Embodiments 141, 142, 143 and 144, preferably of Embodiment 143, or a pharmaceutically acceptable salt, a pharmaceutically acceptable solvate or a hydrate thereof, wherein R^{6f} is H.

Embodiment 146. The compound of any one of Embodiments 141, 142, 143 and 144, preferably of Embodiment 143, or a pharmaceutically acceptable salt, a pharmaceutically acceptable solvate or a hydrate thereof, wherein R^{6f} is CH₃.

Embodiment 147. The compound of any one of Embodiment 143, or a pharmaceutically acceptable salt, a pharmaceutically acceptable solvate or a hydrate thereof, wherein
n is 3, and R^{6e}, R^{6f} and R^{6g} are each and independently selected from the group consisting of CH₃.

Embodiment 148. The compound of any one of Embodiments 141 and 142, or a pharmaceutically acceptable salt, a pharmaceutically acceptable solvate or a hydrate thereof, wherein
n is 3; and
R^{6a} is C(═O)R^{6h}, wherein R^{6h} is NH₂.

Embodiment 149. The compound of any one of Embodiments 123, 124, 125, 126, 127, 128, 129, 130, 131, 132, 133, 134, 135, 136, 137, 138, 139, 140, 141, 142, 143, 144, 145, 146, 147 and 148, wherein
R^{7a} is selected from the group consisting of H, (C₁-C₂)alkyl, CH₂OH, and CH₂CO₂H, and CH₂CH₂CO₂H; and
R^{7b} is selected from the group consisting of H and (C₁-C₂)alkyl.

Embodiment 150. The compound of Embodiment 149, or a pharmaceutically acceptable salt, a pharmaceutically acceptable solvate or a hydrate thereof, wherein R^{7c} is H.

Embodiment 151. The compound of any one of Embodiments 149 and 150, or a pharmaceutically acceptable salt, a pharmaceutically acceptable solvate or a hydrate thereof, wherein
R^{7a} is selected from the group consisting of (C₁-C₂)alkyl, CH₂OH, CH₂CO₂H, and CH₂CH₂CO₂H; and
R^{7b} is selected from the group consisting of (C₁-C₂)alkyl.

Embodiment 152. The compound of Embodiment 151, or a pharmaceutically acceptable salt, a pharmaceutically acceptable solvate or a hydrate thereof, wherein R^{7a} is CH₃; and R^{7b} is CH₃.

Embodiment 153. The compound of Embodiment 151, or a pharmaceutically acceptable salt, a pharmaceutically acceptable solvate or a hydrate thereof, wherein R^{7a} is H; and R^{7b} is CH₃.

Embodiment 154. The compound of any one of Embodiments 123, 124, 125, 126, 127, 128, 129, 130, 131, 132, 133, 134, 135, 136, 137, 138, 139, 140, 141, 142, 143, 144, 145, 146, 147, 148, 149, 150, 151, 152 and 153, or a pharmaceutically acceptable salt, a pharmaceutically acceptable solvate or a hydrate thereof, wherein
R^{8a} is selected from the group consisting of H, OH, COOH, C(═O)NH₂, CH₂CH₂NHC(═NH)NH₂, (C₁-C₈)alkyl, aryl, and heteroaryl;
R^{8b} is selected from the group consisting of H and CH₃.

Embodiment 155. The compound of any one of Embodiments 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153 and 154, or a pharmaceutically acceptable salt, a pharmaceutically acceptable solvate or a hydrate thereof, wherein
R^{8a} is selected from the group consisting of OH, COOH, C(═O)NH₂, CH(CH₃)₂, CH₂CH₂NHC(═NH)NH₂, phenyl, and indole.

Embodiment 156. The compound of Embodiment 155, wherein R^{8b} is H.

Embodiment 157. The compound of any one of Embodiments 154, 155 and 156, or a pharmaceutically acceptable salt, a pharmaceutically acceptable solvate or a hydrate thereof, wherein R^{8a} is C(═O)NH₂.

Embodiment 158. The compound of any one of Embodiments 134, 135, 136, 137, 138, 139, 140, 141, 142, 143, 144, 145, 146, 147, 148, 149, 150, 151, 152, 153, 154, 155, 156 and 157, or a pharmaceutically acceptable salt, a pharmaceutically acceptable solvate or a hydrate thereof, wherein R^{3c} is selected from the group consisting of H, Cl, CH₃, F, CN, and CF₃.

Embodiment 159. The compound of Embodiment 158, or a pharmaceutically acceptable salt, a pharmaceutically acceptable solvate or a hydrate thereof, wherein R^{3c} is H.

Embodiment 160. The compound of Embodiment 158, or a pharmaceutically acceptable salt, a pharmaceutically acceptable solvate or a hydrate thereof, wherein R^{3c} is Cl and is in the para position.

Embodiment 161. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 and 33, or a pharmaceutically acceptable salt, a pharmaceutically acceptable solvate or a hydrate thereof, wherein
Xaa2 is Aib or Ala;
Xaa3 is Phe or Pcf;
Xaa5 is Lys(Me), Lys, Kip or KMe3;
Xaa6 is Arg(Me), Arg, Egd, Cit, RMe2a or RMe3;
Xaa7 is Aib or Ala;
Xaa8 is Asn; and
Xaa10 is Tle or Leu.

Embodiment 162. The compound of Embodiment 161, or a pharmaceutically acceptable salt, a pharmaceutically acceptable solvate or a hydrate thereof, wherein
Xaa2 is Aib or Ala;
Xaa3 is Phe or Pcf;
Xaa5 is Lys(Me), Lys or Kip;
Xaa6 is Arg(Me), Arg, Egd, Cit or RMe2a;
Xaa7 is Aib or Ala;
Xaa8 is Asn; and
Xaa10 is Tle or Leu.

Embodiment 163. The compound of Embodiment 161, or a pharmaceutically acceptable salt, a pharmaceutically acceptable solvate or a hydrate thereof, wherein
Xaa2 is Aib or Ala;
Xaa3 is Phe or Pcf;
Xaa5 is Lys(Me) or Lys;
Xaa6 is Arg(Me) or Arg;
Xaa7 is Aib or Ala;
Xaa8 is Asn; and
Xaa10 is Tle or Leu.

Embodiment 164. The compound of Embodiment 161, or a pharmaceutically acceptable salt, a pharmaceutically acceptable solvate or a hydrate thereof, wherein
Xaa2 is Aib or Ala;
Xaa3 is Phe or Pcf;
Xaa5 is Lys(Me) or Lys;
Xaa6 is Arg(Me) or Arg;
Xaa7 is Aib or Ala;
Xaa8 is Asn; and
Xaa10 is Tle.

Embodiment 165. The compound of any one of Embodiments 161, 162, 163, 164 and 165, or a pharmaceutically acceptable salt, a pharmaceutically acceptable solvate or a hydrate thereof, wherein
Z¹ is chelator; and
L¹ is Formula (XIII)

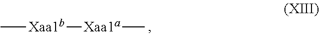

wherein Xaa1^{a} is covalently bound to Xaa2;
Xaa1^{a} is Thr; and
Xaa1^{b} is Cmp.

Embodiment 166. The compound of any one of Embodiments 161, 162, 163 and 164, or pharmaceutically acceptable salt, solvate or hydrate thereof, wherein
Z¹ is chelator; and
L¹ is Pamb.

Embodiment 167. The compound of any one of Embodiments 161, 162, 163, 164, 165 and 166, or pharmaceutically acceptable salt, solvate or hydrate thereof, wherein
Z² is NH₂; and
L² is Thr.

Embodiment 168. The compound of any one of Embodiments 161, 162, 163, 164, 165 and 166, or pharmaceutically acceptable salt, solvate or hydrate thereof, wherein
Z² is NH₂; and
L² is Bal.

Embodiment 169. The compound of any one of Embodiments 161, 162, 163, 164, 165 and 166, or pharmaceutically acceptable salt, solvate or hydrate thereof, wherein
Z² is NH₂; and
L² is Thr-Asp.

Embodiment 170. The compound of any one of Embodiments 161, 162, 163, 164, 165 and 166, or pharmaceutically acceptable salt, solvate or hydrate thereof, wherein
Z² is NH₂; and
L² is bond.

Embodiment 171. The compound of any one of Embodiments 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 and 66, or pharmaceutically acceptable salt, solvate or hydrate thereof, wherein
Xaa2 is Aib or Ala;
Xaa3 is Phe or Pcf;
Xaa5 is Lys or Lys(Me);
Xaa6 is Arg or Arg(Me);
Xaa7 is Aib or Ala;
Xaa8 is Ans; and
Xaa10 is Tle or Leu.

Embodiment 172. The compound of Embodiment 171, or pharmaceutically acceptable salt, solvate or hydrate thereof, wherein
Z¹ is Ac; and
L¹ is Thr.

Embodiment 173. The compound of Embodiment 171, or pharmaceutically acceptable salt, solvate or hydrate thereof, wherein
Z¹ is Ac; and
L¹ is bond.

Embodiment 174. The compound of Embodiment 171, or pharmaceutically acceptable salt, solvate or hydrate thereof, wherein
Z¹ is Ac; and
L¹ is Formula (XIII)

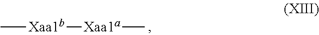

wherein Xaa1^{a} is covalently bound to Xaa2 of Formula (I);
Xaa1^{a} is Thr; and
Xaa1^{b} is Met or Cmp.

Embodiment 175. The compound of any one of Embodiments 171, 172, 173 and 174, or pharmaceutically acceptable salt, solvate or hydrate thereof, wherein
Z2 is chelator; and
L2 is en.

Embodiment 176. The compound of any one of Embodiments 171, 172, 173 and 174, or pharmaceutically acceptable salt, solvate or hydrate thereof, wherein
Z2 is chelator; and
L2 is -Xaa11-Xaa12^{a}-Xaa12^{b}-; wherein
Xaa11 is Thr;
Xaa12^{a} is Asp; and
Xaa12^{b} is Ttds.

Embodiment 177. The compound of any one of Embodiments 171, 172, 173 and 174, or pharmaceutically acceptable salt, solvate or hydrate thereof, wherein
Z2 is chelator; and
L2 is -Xaa11-Xaa12; wherein
Xaa11 is Thr;
Xaa12^{a} is Cmp; and
Xaa12^{b} is Ttds.

Embodiment 178. The compound of any one of Embodiments 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176 and 177, or pharmaceutically acceptable salt, solvate or hydrate thereof, wherein chelator is selected from the group consisting of DOTA, DOTAGA, LSC, NOPO, PCTA, DOTAM, Macropa, Crown, NOTA, and NODAGA.

Embodiment 179. The compound of Embodiment 178, or pharmaceutically acceptable salt, solvate or hydrate thereof, wherein chelator is selected from the group consisting of DOTA and DOTAGA.

Embodiment 180. The compound of Embodiment 179, or pharmaceutically acceptable salt, solvate or hydrate thereof, wherein the chelator is DOTA.

Embodiment 181. The compound of Embodiment 1, or pharmaceutically acceptable salt, solvate or hydrate thereof, wherein the compound is selected from the following

DOTA-Cmp-Thr-Aib-Pcf-[Cys-Lys(Me)-Arg(Me)-Aib-Asn-Cys]-Tle-Thr-NH₂ (PSM-0194);
DOTA-Cmp-Thr-Aib-Pcf-[Cys-Lys-Arg-Aib-Asn-Cys]-Tle-NH₂ (PSM-0433);
DOTA-Cmp-Thr-Aib-Phe-[Cys-Lys-Arg-Aib-Asn-Cys]-Tle-Thr-NH₂ (PSM-0492);
Ac-Thr-Aib-Phe-[Cys-Lys-Arg-Aib-Asn-Cys]-Tle-Thr-NH₂ (PSM-0178);
Ac-Thr-Aib-Pcf-[Cys-Lys-Cit-Aib-Asn-Cys]-Tle-Thr-NH₂ (PSM-0179);
Ac-Thr-Aib-Phe-[Cys-Lys-Gln-Ala-Asn-Cys]-Tle-Thr-Asp-NH₂ (PSM-0180);
Ac-Thr-Aib-Phe-[Cys-Lys-Arg-Hyp-Asn-Cys]-Tle-Thr-Asp-NH₂ (PSM-0181);
Ac-Thr-Aib-Phe-[Cys-Lys-Arg-Aib-Asn-Cys]-Tle-Thr-Asp-Ttds-Ttds-AF488N3K-NH₂ (PSM-0183);
DOTA-Thr-Aib-Phe-[Cys-Lys-Arg-Aib-Asn-Cys]-Tle-NH₂ (PSM-0184);
Ac-Pamp-Aib-Pcf-[Cys-Lys-Arg-Aib-Asn-Cys]-Tle-en-DOTA (PSM-0186);
Ac-Thr-Deg-Phe-[Cys-Lys-Arg-Aib-Asn-Cys]-Tle-Thr-NH₂ (PSM-0187);
SaPr-Aib-Pcf-[Cys-Lys-Arg-Lys(DOTA-APAc)-Asn-Cys]-Tle-Thr-NH₂ (PSM-0188);
Ac-Thr-Aib-Pcf-[Cys-Lys-Gln(Gu)-Aib-Asn-Cys]-Tle-Thr-NH₂ (PSM-0189);
Ac-Thr-Aib-Pcf-[Cys-Lys-Arg-Aib-Asn-Cys]-Tle-Thr-lys(DOTA-Cmp)-NH₂ (PSM-0190);
Ac-Thr-Aib-Phe-[Cys-Lys-Arg-Aib-Asn-Cys]-Tle-Thr-APAc-lys(DOTA)-NH₂ (PSM-0191);
DOTA-Ttds-Thr-Aib-Phe-[Cys-Lys-Arg-Ala-Asn-Cys]-Tle-Thr-Asp-NH₂ (PSM-0193);
Ac-Thr-Ala-Phe-[Cys-Lys-Arg-Glu-Asn-Cys]-Nle-Thr-Asp-NH₂ (PSM-0197);
DOTA-Cmp-Tle-Aib-Phe-[Cys-Lys-Arg-Aib-Asn-Cys]-Tle-Thr-NH₂ (PSM-0198);
DOTA-Thr-Aib-Phe-[Cys-Lys-Arg-Aib-Asn-Cys]-Tle-en (PSM-0199);
DOTA-Cmp-Thr-Aib-Pcf-[Cys-Lys(Me)-Arg-Aib-Asn-Cys]-Tle-NH₂ (PSM-0200);
DOTA-Pamb-Thr-Aib-Phe-[Cys-Lys-Arg-Aib-Asn-Cys]-Tle-Thr-NH₂ (PSM-0202);
DOTA-Cmp-Thr-Aib-Pcf-[Cys-KMe2-Arg(Me)-Aib-Asn-Cys]-Tle-Thr-NH₂ (PSM-0203);
nBuCAyl-Thr-Aib-Pcf-[Cys-Lys-Cit-Aib-Asn-Cys]-Tle-Thr-Cmp-lys(DOTA)-NH₂ (PSM-0204);
Ac-Thr-Aib-Pcf-[Cys-Nle-Arg-Aib-Asn-Cys]-Tle-Thr-NH₂ (PSM-0205);
nBuCAyl-Thr-Aib-Mcf-[Cys-Lys-Cit-Aib-Asn-Cys]-Tle-Thr-Cmp-lys(DOTA)-NH₂ (PSM-0207);
Ac-Thr-Aib-Phe-[Cys-Lys-Arg-Aib-Asn-Cys]-NHnPen (PSM-0208);
DOTA-Bal-Thr-Aib-Phe-[Cys-Lys-Arg-Aib-Asn-Cys]-Tle-Thr-NH₂ (PSM-0209);
Ac-Thr-Aib-Phe-[Cys-Gln-Arg-Aib-Asn-Cys]-Tle-Thr-Asp-NH₂ (PSM-0210);
Ac-Thr-Aib-Phe-[Cys-Lys-Arg-Lys(DOTA-Pab)-Asn-Cys]-Tle-Thr-NH₂ (PSM-0211);
Ac-Thr-Aib-Phe-[Cys-Lys-Arg-Aib-Asn-Cys]-Tle-Thr-Asp-Ttds-lys(DOTA)-NH₂ (PSM-0212);
SaPr-Aib-Pcf-[Cys-Lys-Arg-Aib-Asn-Cys]-Tle-Thr-Cmp-lys(DOTA)-NH₂ (PSM-0215);
DOTA-Thr-Aib-Pcf-[Cys-Lys-Arg-Aib-Asn-Cys]-Tle-Bal-NH₂ (PSM-0216);
Ac-Thr-Aib-Phe-[Cys-Lys-Arg-Dfp-Asn-Cys]-Tle-Thr-Asp-NH₂ (PSM-0217);
DOTAGA-Cmp-Thr-Aib-Phe-[Cys-Lys-Arg-Aib-Asn-Cys]-Tle-Thr-NH₂ (PSM-0218);
DOTA-Cmp-Leu-Aib-Phe-[Cys-Lys-Arg-Aib-Asn-Cys]-Tle-Thr-NH₂ (PSM-0220);
DOTA-Pamb-Aib-Phe-[Cys-Lys-Arg-Aib-Asn-Cys]-Tle-en (PSM-0221);
DOTA-Thr-Aib-Pcf-[Cys-Lys-Arg-Aib-Asn-Cys]-Tle-en (PSM-0222);
DOTA-Cmp-Aib-Pcf-[Cys-Lys-Arg(Ac)-Aib-Asn-Cys]-Tle-Thr-NH₂ (PSM-0223);
SaPr-Aib-Phe-[Cys-Lys-Arg-Aib-Asn-Cys]-Tle-Thr-NH₂ (PSM-0224);
DOTA-Cmp-Thr-Aib-Pcf-[Cys-Har-Arg-Aib-Asn-Cys]-Tle-Thr-NH₂ (PSM-0225);
H-Met-Thr-Ala-Phe-[Cys-Lys-Arg-Ala-Asn-Cys]-Leu-Thr-Asp-Gly-Ser-Ttds-Ttds-Ttds-
Lys(Bio)-NH₂ (PSM-0226);
DOTA-Cmp-Thr-Aib-Pcf-[Cys-Lys-Arg-amd-Asn-Cys]-Tle-Thr-NH₂ (PSM-0227);
Ac-Aib-Phe-[Cys-Lys-Arg-Aib-Asn-Cys]-Tle-Thr-NH₂ (PSM-0228);
Ac-Thr-Aib-Pcf-[Cys-Lys-Arg-Aib-Met-Cys]-Tle-Thr-NH₂ (PSM-0229);
Ac-Thr-Aib-Phe-[Cys-Lys-Arg-Aib-Asn-Cys]-Tle-NH₂ (PSM-0230);
Ac-Thr-Aib-Phe-[Cys-Lys-Arg-Ala-Asn-Cys]-Tle-Thr-Ttds-lys(DOTA)-NH₂ (PSM-0231);
Ac-Thr-Aib-Phe-[Cys-Lys-Arg-Nmg-Asn-Cys]-Tle-Thr-NH₂ (PSM-0232);
Ac-Thr-Aib-Phe-[Cys-Lys-Arg-Pam-Asn-Cys]-Tle-Thr-NH₂ (PSM-0233);
DOTA-Ttds-Thr-Aib-Phe-[Cys-Lys-Cit-Aib-Asn-Cys]-Tle-Thr-NH₂ (PSM-0234);
Ac-Thr-Aib-Phe-[Cys-Lys-Nmr-Aib-Asn-Cys]-Tle-Thr-NH₂ (PSM-0235);
Ac-Thr-Aib-Pcf-[Cys-Nle-Gln(Gu)-Aib-Asn-Cys]-Tle-Thr-NH₂ (PSM-0236);
DOTA-Ttds-Thr-Aib-Phe-[Cys-Lys-Arg-Ala-Asn-Cys]-Tle-Thr-NH₂ (PSM-0237);
Ac-Thr-Aib-Pcf-[Cys-Lys-Cit-Aib-Trp-Cys]-Tle-Thr-NH₂ (PSM-0238);
Hex-Thr-Aib-Pcf-[Cys-Lys-Cit-Aib-Asn-Cys]-Tle-Thr-lys(DOTA-Cmp)-NH₂ (PSM-0239);
Ac-Thr-Aib-Pcf-[Cys-Lys-Cit-Aib-Arg-Cys]-Tle-Thr-NH₂ (PSM-0240);
DOTA-Cmp-Thr-Aib-Phe-[Cys-Lys-Arg-Aib-Asn-Cys]-Tle-en (PSM-0241);
Ac-Thr-Aib-Mcf-[Cys-Lys-Arg-Lys(DOTA-O20c)-Asn-Cys]-Tle-Thr-NH₂ (PSM-0243);
DOTA-Cmp-Thr-Aib-Pcf-[Cys-Kip-Arg(Me)-Aib-Asn-Cys]-Tle-Thr-NH₂ (PSM-0244);
DOTA-Cmp-Thr-Aib-Pcf-[Cys-Lys-Orn-Aib-Asn-Cys]-Tle-Thr-NH₂ (PSM-0245);
DOTA-Cmp-Thr-Aib-Pcf-[Cys-Lys(Me)-Arg(Me)-ala-Asn-Cys]-Tle-Thr-NH₂ (PSM-0246);
DOTA-Cmp-Thr-Aib-Pcf-[Cys-Lys(Me)-RMe2-Aib-Asn-Cys]-Tle-Thr-NH₂ (PSM-0247);
DOTA-Cmp-Aib-Pcf-[Cys-Lys-Arg-Aib-Asn-Cys]-Tle-Thr-NH₂ (PSM-0248);
Ac-Thr-Aib-1Ni-[Cys-Lys-Arg-Aib-Asn-Cys]-Tle-Thr-Asp-NH₂ (PSM-0249);
DOTA-Cmp-Thr-Aib-Pcf-[Cys-Lys-Arg-Aib-Asn-Cys]-Tle-Thr-Ttds-Ttds-Lys(Bio)-NH₂ (PSM-0250);
DOTA-Ttds-Thr-Ala-Phe-[Cys-Lys-Arg-Ala-Asn-Cys]-Leu-Thr-Asp-NH₂ (PSM-0251);
Ac-Thr-Aib-Phe-[Cys-Lys-Cit-Aib-Asn-Cys]-Tle-Thr-Cmp-lys(DOTA)-NH₂ (PSM-0252);
Ac-Thr-Aib-Phe-[Cys-Lys-Glu-Aib-Asn-Cys]-Tle-Thr-Asp-NH₂ (PSM-0253);
DOTA-Cmp-Thr-Aib-Pcf-[Cys-Lys(Me)-Eew-Aib-Asn-Cys]-Tle-Thr-NH₂ (PSM-0254);
DOTA-Cmp-Thr-Aib-Mpa-[Cys-Lys-Cit-Aib-Asn-Cys]-Tle-Thr-NH₂ (PSM-0255);
DOTA-Cmp-Thr-ams-Pcf-[Cys-Lys-Arg-Aib-Asn-Cys]-Tle-Thr-NH₂ (PSM-0256);
DOTA-Cmp-Thr-Aib-Opa-[Cys-Lys(Me)-Arg(Me)-Aib-Asn-Cys]-Tle-Thr-NH₂ (PSM-0257);
nBuCAyl-Thr-Aib-Phe-[Cys-Lys-Opy-Aib-Asn-Cys]-Tle-Thr-Cmp-lys(DOTA)-NH₂ (PSM-0258);
DOTA-Ttds-Thr-Aib-Phe-[Cys-Lys-Arg-Aib-Asn-Cys]-Tle-Thr-NH₂ (PSM-0259);
DOTA-Cmp-Thr-Aib-Pcf-[Cys-Lys-Arg-glu-Asn-Cys]-Tle-Thr-NH₂ (PSM-0260);
DOTA-APAc-Aib-Phe-[Cys-Lys-Arg-Aib-Asn-Cys]-Tle-Thr-NH₂ (PSM-0261);
Ac-Thr-Aib-Phe-[Cys-Lys-Arg-Aib-Asn-Cys]-Tle-Glu-NH₂ (PSM-0262);
DOTA-APAc-Aib-Pcf-[Cys-Lys-Arg-Aib-Asn-Cys]-Tle-Thr-NH₂ (PSM-0263);
Ac-Thr-Aib-Mtf-[Cys-Lys-Arg-Aib-Asn-Cys]-Tle-Thr-NH₂ (PSM-0264);
Ac-Thr-Ala-Phe-[Cys-Lys-Arg-Ala-Asn-Cys]-Tle-Thr-Asp-Ttds-lys(DOTA)-NH₂ (PSM-0266);
HPA-Aib-Pcf-[Cys-Lys-Arg-Aib-Asn-Cys]-Tle-Thr-lys(DOTA)-NH₂ (PSM-0267);
Ac-Thr-Aib-Pcf-[Cys-Lys-Arg-Aib-Asn-Cys]-Tle-en-DOTA (PSM-0269);
DOTA-Pamb-Aib-Phe-[Cys-Lys-Arg-Aib-Asn-Cys]-Tle-Thr-NH₂ (PSM-0270);
AF488Ahx-Ttds-Ttds-Thr-Aib-Phe-[Cys-Lys-Arg-Aib-Asn-Cys]-Tle-Thr-Asp-NH₂ (PSM-0272);
DOTA-Pamb-Aib-Pcf-[Cys-Lys-Arg-Aib-Asn-Cys]-Tle-en (PSM-0273);
DOTA-Cmp-Thr-Aib-Eaa-[Cys-Lys-Cit-Aib-Asn-Cys]-Tle-Thr-NH₂ (PSM-0274);
Ac-Thr-Aib-Mcf-[Cys-Lys-Cit-Aib-Asn-Cys]-Tle-Thr-Cmp-lys(DOTA)-NH₂ (PSM-0275);
Ac-Thr-Aib-Pnf-[Cys-Lys-Arg-Aib-Asn-Cys]-Tle-Thr-NH₂ (PSM-0278);
Ac-Thr-Aib-Phe-[Cys-Lys-Arg-Aib-Ser-Cys]-Tle-Thr-NH₂ (PSM-0279);
Ac-Thr-Aib-Phe-[Cys-Lys-Cit-Aib-Asn-Cys]-Tle-Thr-Asp-NH₂ (PSM-0280);
Ac-Aib-Pcf-[Cys-Lys-Arg-Lys(DOTA-APAc)-Asn-Cys]-Tle-Thr-NH₂ (PSM-0282);
DOTA-Pamb-Aib-Pcf-[Cys-Lys-Arg-Aib-Asn-Cys]-Tle-Thr-NH₂ (PSM-0283);
Ac-Thr-Aib-Mcf-[Cys-Lys-Cit-Lys(DOTA-O20c)-Asn-Cys]-Tle-Thr-NH₂ (PSM-0284);
Ac-Thr-Aib-Phe-[Cys-Lys-Arg-Aib-Asn-Cys]-Tle-Thr-Ttds-lys(DOTA)-NH₂ (PSM-0285);
Ac-Thr-Ala-Phe-[Cys-Lys-Arg-Ala-Asn-Cys]-Nle-Thr-Asp-NH₂ (PSM-0287);
Ac-Thr-Aib-Phe-[Cys-Lys-Arg-Aib-Asn-Cys]-Tle-Thr-lys(DOTA)-NH₂ (PSM-0288);
Hex-Thr-Aib-Phe-[Cys-Lys-Gln-Aib-Asn-Cys]-Tle-Thr-Ttds-lys(DOTA)-NH₂ (PSM-0289);
Iva-Aib-Phe-[Cys-Lys-Arg-Aib-Asn-Cys]-Tle-Thr-lys(DOTA)-NH₂ (PSM-0292);
Ac-Thr-Aib-Pcf-[Cys-Lys-Cit-Aib-Ser-Cys]-Tle-Thr-NH₂ (PSM-0293);
Ac-Thr-Aib-Pff-[Cys-Lys-Arg-Aib-Asn-Cys]-Tle-Thr-NH₂ (PSM-0294);
Ac-Thr-Aib-Phe-[Cys-Lys-Arg-Ala-Asn-Cys]-Tle-Thr-Asp-Ttds-lys(DOTA)-NH₂ (PSM-0295);
DOTA-Cmp-Thr-Aib-Mpa-[Cys-Lys(Me)-Arg(Me)-Aib-Asn-Cys]-Tle-Thr-NH₂ (PSM-0296);
Ac-Thr-Aib-Phe-[Cys-Lys-Arg-Aib-Asn-Cys]-Tle-Gab-NH₂ (PSM-0297);
Ac-Aib-Pcf-[Cys-Lys-Arg(Ac)-Aib-Asn-Cys]-Tle-Thr-Cmp-lys(DOTA)-NH₂ (PSM-0298);
Ac-Ala-Phe-[Cys-Lys-Arg-Ala-Asn-Cys]-Leu-Thr-Asp-Ttds-lys(DOTA)-NH₂ (PSM-0299);
Ac-Thr-Aib-Phe-[Cys-Lys-Arg-Aib-Asn-Cys]-Tle-OH (PSM-0300);
Ac-Thr-Aib-Mcf-[Cys-Lys-Arg-Lys(DOTAGA-O20c)-Asn-Cys]-Tle-Thr-NH₂ (PSM-0301);
DOTA-Cmp-Thr-Aib-Pcf-[Cys-Lys-Arg-Amd-Asn-Cys]-Tle-Thr-NH₂ (PSM-0302);
Ac-Thr-Aib-Phe-[Cys-Lys-Gln-Aib-Asn-Cys]-Tle-Thr-Ttds-lys(DOTA)-NH₂ (PSM-0303);
DOTA-Cmp-Thr-Aib-6Clw-[Cys-Lys-Cit-Aib-Asn-Cys]-Tle-Thr-NH₂ (PSM-0304);
DOTA-Ttds-Thr-Ala-Phe-[Cys-Lys-Arg-Ala-Asn-Cys]-Leu-Thr-NH₂ (PSM-0305);
DOTA-Cmp-Thr-Aib-Pcf-[Cys-Lys-Arg-Aib-Asn-Cys]-Tle-en(Me)2 (PSM-0306);
Ac-Thr-Aib-Phe-[Cys-Lys-Cit-Ala-Asn-Cys]-Tle-Thr-Asp-NH₂ (PSM-0307);
DOTA-Cmp-Thr-Aib-5Clw-[Cys-Lys(Me)-Arg(Me)-Aib-Asn-Cys]-Tle-Thr-NH₂ (PSM-0308);
DOTA-Ahx-Aib-Pcf-[Cys-Lys-Arg-Aib-Asn-Cys]-Tle-Thr-NH₂ (PSM-0310);
Ac-Thr-Ala-Phe-[Cys-Lys-Arg-Ala-Asn-Cys]-Nle-Thr-Gab-OH (PSM-0313);
DOTA-Cmp-Thr-Aib-Pcf-[Cys-Lys-Arg-ams-Asn-Cys]-Tle-Thr-NH₂ (PSM-0314);
Ac-Thr-Aib-Phe-[Cys-Lys-arg-Ala-Asn-Cys]-Tle-Thr-Asp-NH₂ (PSM-0315);
DOTA-Pab-Aib-Phe-[Cys-Lys-Arg-Aib-Asn-Cys]-Tle-Thr-NH₂ (PSM-0316);
DOTA-Cmp-Thr-Aib-Pcf-[Cys-Lys-Har-Aib-Asn-Cys]-Tle-Thr-NH₂ (PSM-0317);
Ac-Thr-Aib-Pcf-[Cys-Lys-Arg-Aib-Asn-Cys]-Tle-Thr-Asp-NH₂ (PSM-0318);
Ac-Thr-Ala-Nmf-[Cys-Lys-Arg-Aib-Asn-Cys]-Tle-Thr-NH₂ (PSM-0319);
Ac-Aib-Pcf-[Cys-Lys-Arg(Me)-Aib-Asn-Cys]-Tle-en-DOTA (PSM-0320);
Ac-Thr-Aib-Phe-[Cys-Lys-Arg-Apc(DOTA)-Asn-Cys]-Tle-Thr-NH₂ (PSM-0321);
Ac-Thr-Aib-Pcf-[Cys-Lys-Cit-Aib-Asn-Cys]-Tle-Thr-lys(DOTA-Cmp)-NH₂ (PSM-0322);
HPA-Aib-Pcf-[Cys-Lys-Arg-Lys(DOTA-APAc)-Asn-Cys]-Tle-Thr-NH₂ (PSM-0324);
Ac-Thr-Aib-Phe-[Cys-Lys-Arg-Lys(DOTA)-Asn-Cys]-Tle-Thr-NH₂ (PSM-0326);
SaPr-Aib-Pcf-[Cys-Lys-Arg-Aib-Asn-Cys]-Tle-Thr-lys(DOTA)-NH₂ (PSM-0328);
Ac-Thr-Aib-Pcf-[Cys-Lys-Cit-Aib-Asn-Cys]-Tle-Thr-Cmp-lys(DOTA)-NH₂ (PSM-0329);
Ac-Thr-Ala-Phe-[Cys-Lys-Arg-Ala-Asn-Cys]-Leu-Thr-Asp-Ttds-lys(DOTA)-NH₂ (PSM-0330);
DOTA-Cmp-Thr-Aib-Pcf-[Smc-Lys-Arg-Aib-Asn-Cys]-Tle-NH₂
(alternative: DOTA-Cmp-Thr-Aib-Pcf-[Cys-Lys-Arg-Aib-Asn-Smc]-Tle-NH₂) (PSM-0332);
Ac-Thr-Aib-Phe-[Cys-Lys-Gln-Aib-Asn-Cys]-Tle-Thr-Asp-NH₂ (PSM-0334);
Ac-Thr-Ala-Phe-[Cys-Lys-Arg-Ala-Ala-Cys]-Nle-Thr-Asp-NH₂ (PSM-0335);
Ac-Thr-Aib-Pcf-[Cys-Lys-Arg-Aib-Asn-Cys]-Tle-Thr-NH₂ (PSM-0336);
Ac-Thr-Aib-Pcf-[Cys-Lys-Nle-Aib-Asn-Cys]-Tle-Thr-NH₂ (PSM-0338);
Ac-Thr-Aib-Phe-[Cys-Lys-Arg-Aib-Asn-Cys]-Tle-Thr-Cmp-lys(DOTAGA)-NH₂ (PSM-0339);
DOTA-Cmp-Thr-Aib-Pcf-[Cys-Lys-Nmr-Aib-Asn-Cys]-Tle-Thr-NH₂ (PSM-0340);
DOTA-Cmp-Thr-Aib-Pcf-[Cys-Lys-Lys(Ac)-Aib-Asn-Cys]-Tle-Thr-NH₂ (PSM-0341);
DOTA-Ttds-Thr-Ala-Phe-[Cys-Lys-Arg-Ala-Asn-Cys]-Tle-Thr-Asp-NH₂ (PSM-0342);
DOTA-Cmp-Thr-Aib-Pcf-[Cys-Lys-Arg(Me)-Aib-Asn-Cys]-Tle-NH₂ (PSM-0345);
DOTA-Cmp-Thr-Amd-Pcf-[Cys-Lys-Arg-Aib-Asn-Cys]-Tle-Thr-NH₂ (PSM-0346);
Ac-Aib-Pcf-[Cys-Lys-Arg-Aib-Asn-Cys]-Tle-Thr-Cmp-lys(DOTA)-NH₂ (PSM-0349);
Ac-Thr-Aib-Phe-[Cys-Lys-Arg-Aib-Asn-Cys]-leu-Thr-NH₂ (PSM-0350);
DOTA-Cmp-Thr-Aib-Pcf-[Cys-Lys-Arg(Me)-Aib-Asn-Cys]-Tle-Thr-NH₂ (PSM-0351);
DOTA-Bal-Aib-Pcf-[Cys-Lys-Arg-Aib-Asn-Cys]-Tle-Thr-NH₂ (PSM-0352);
Iva-Aib-Pcf-[Cys-Lys-Arg-Lys(DOTA-APAc)-Asn-Cys]-Tle-Thr-NH₂ (PSM-0353);
DOTA-Cmp-Thr-Aib-Mcf-[Cys-Lys(Me)-Arg(Me)-Aib-Asn-Cys]-Tle-NH₂ (PSM-0354);
DOTA-Cmp-Thr-Aib-Pcf-[Cys-Lys(Me)-RMe3-Aib-Asn-Cys]-Tle-Thr-NH₂ (PSM-0355);
DOTA-Cmp-Ile-Aib-Phe-[Cys-Lys-Arg-Aib-Asn-Cys]-Tle-Thr-NH₂ (PSM-0357);
DOTA-Cmp-Thr-Aib-Phe-[Cys-Lys-Arg-Aib-Asn-Cys]-Tle-NH₂ (PSM-0361);
Ac-Thr-Aib-Phe-[Cys-Lys-Glu-Ala-Asn-Cys]-Tle-Thr-Asp-NH₂ (PSM-0363);
DOTA-Cmp-Thr-Aib-Pcf-[Cys-Lys-Cit-Aib-Asn-Cys]-Tle-Thr-NH₂ (PSM-0365);
DOTA-Cmp-Thr-amd-Pcf-[Cys-Lys-Arg-Aib-Asn-Cys]-Tle-Thr-NH₂ (PSM-0366);
Ac-Thr-Aib-Pcf-[Cys-Lys-Arg-Aib-Asn-Cys]-Tle-NH₂ (PSM-0367);
DOTA-Ahx-Thr-Aib-Phe-[Cys-Lys-Arg-Aib-Asn-Cys]-Tle-Thr-NH₂ (PSM-0368);
DOTA-Cmp-Thr-Ams-Pcf-[Cys-Lys-Arg-Aib-Asn-Cys]-Tle-Thr-NH₂ (PSM-0369);
Ac-Thr-Aib-Pcf-[Cys-Lys-Arg-Lys(DOTA-O20c)-Asn-Cys]-Tle-Thr-NH₂ (PSM-0370);
nBuCAyl-Thr-Aib-Pcf-[Cys-Lys-Arg-Aib-Asn-Cys]-Tle-Thr-Cmp-lys(DOTA)-NH₂ (PSM-0371);
Ac-Thr-Aib-Pmf-[Cys-Lys-Arg-Aib-Asn-Cys]-Tle-Thr-NH₂ (PSM-0372);
Ac-Thr-Aib-Phe-[Cys-Lys-Arg-Aib-Asn-Cys]-Tle-Thr-Asp-NH₂ (PSM-0374);
Ac-Thr-Aib-Phe-[Cys-Lys-Arg-Deg-Asn-Cys]-Tle-Thr-NH₂ (PSM-0375);
Ac-Thr-Aib-Phe-[Cys-Lys-Arg-Aib-Asn-Cys]-Tle-Trp-NH₂ (PSM-0376);
DOTA-Cmp-Thr-Aib-Pcf-[Cys-Lys-Arg-Aib-Asn-Cys]-Val-Nmt-NH₂ (PSM-0377);
H-Cmp-Thr-Aib-Phe-[Cys-Lys-Arg-Aib-Asn-Cys]-Tle-Thr-Cmp-lys(DOTA)-NH₂ (PSM-0378);
Ac-Cmp-Thr-Aib-Pcf-[Cys-Lys-Arg-Aib-Asn-Cys]-Tle-en-DOTA (PSM-0379);
DOTA-Cmp-Thr-Aib-Pcf-[Cys-Lys(Me)-Arg(Me)-Aib-Asn-Cys]-Tle-NH₂ (PSM-0380);
DOTA-Pamb-Aib-Pcf-[Cys-Lys-Arg-Aib-Asn-Cys]-Tle-NH₂ (PSM-0381);
DOTA-Cmp-Thr-Aib-Pcf-[Cys-Lys-Arg-Ams-Asn-Cys]-Tle-Thr-NH₂ (PSM-0382);
Ac-Thr-Aib-Mcf-[Cys-Lys-Arg-Aib-Asn-Cys]-Tle-Thr-Asp-NH₂ (PSM-0383);
DOTA-Cmp-Thr-Aib-Pcf-[Cys-Lys-Arg-Aib-Asn-Cys]-Tle-en (PSM-0384);
Ac-Thr-Aib-Phe-[Cys-Lys-Gln-Lys(DOTA)-Asn-Cys]-Tle-Thr-NH₂ (PSM-0385);
Ac-Thr-Aib-Phe-[Cys-Lys-Arg-Aib-Asn-Cys]-Tle-Pro-NH₂ (PSM-0388);
Ac-Thr-Ala-Phe-[Cys-Lys-Arg-Ala-Asn-Cys]-Nle-Thr-asp-NH₂ (PSM-0389);
DOTA-Cmp-Thr-Aib-6Clw-[Cys-Lys(Me)-Arg(Me)-Aib-Asn-Cys]-Tle-NH₂ (PSM-0390);
Ac-Thr-Aib-Phe-[Cys-Lys-Arg-Aib-Asn-Cys]-Tle-Thr-Ape-DOTA (PSM-0391);
Ac-Thr-Aib-Phe-[Cys-Lys-Arg-4Tfp-Asn-Cys]-Tle-Thr-Asp-NH₂ (PSM-0392);
Ac-Thr-Aib-Pcf-[Cys-Lys-Arg-Aib-Asn-Cys]-Tle-en (PSM-0393);
Ac-Thr-Aib-Phe-[Cys-Lys-Arg-Aib-Asn-Cys]-Aib-Thr-NH₂ (PSM-0394);
DOTA-Cmp-Thr-Aib-Pff-[Cys-Lys(Me)-Arg(Me)-Aib-Asn-Cys]-Tle-NH₂ (PSM-0395);
DOTA-Cmp-Thr-Aib-Mtf-[Cys-Lys(Me)-Arg(Me)-Aib-Asn-Cys]-Tle-NH₂ (PSM-0396);
Ac-Thr-Aib-Phe-[Cys-Lys-Arg-Lys(DOTA)-Asn-Cys]-Tle-Thr-Asp-NH₂ (PSM-0397);
DOTA-Ttds-Thr-Aib-Phe-[Cys-Lys-Arg-Aib-Asn-Cys]-Tle-Thr-Asp-NH₂ (PSM-0398);
Hex-Thr-Aib-Pcf-[Cys-Lys-Arg-Aib-Asn-Cys]-Tle-Thr-lys(DOTA-Cmp)-NH₂ (PSM-0400);
Ac-Thr-Aib-Phe-[Cys-Lys-Arg-Lys(DOTA-O20c)-Asn-Cys]-Tle-Thr-NH₂ (PSM-0401);
Ac-Thr-Aib-Phe-[Cys-Lys-Arg-Aib-asn-Cys]-Tle-Thr-NH₂ (PSM-0402);
Ac-Thr-Aib-Phe-[Cys-Lys-Arg-Oic-Asn-Cys]-Tle-Thr-Asp-NH₂ (PSM-0403);
Ac-Aib-Pcf-[Cys-Lys-Arg-Aib-Asn-Cys]-Tle-Thr-lys(DOTA)-NH₂ (PSM-0404);
Hex-Thr-Ala-Phe-[Cys-Lys-Arg-Ala-Asn-Cys]-Leu-Thr-Asp-Ttds-lys(DOTA)-NH₂ (PSM-0405);
DOTA-Cmp-Thr-Ams-Pcf-[Cys-Lys-Arg-Ams-Asn-Cys]-Tle-Thr-NH₂ (PSM-0407);
Ac-Thr-Nmg-Phe-[Cys-Lys-Arg-Aib-Asn-Cys]-Tle-Thr-NH₂ (PSM-0408);
DOTA-Cmp-Thr-Aib-Opa-[Cys-Lys(Me)-Arg(Me)-Aib-Asn-Cys]-Tle-NH₂ (PSM-0409);
DOTA-Cmp-Thr-Aib-Pcf-[Cys-Lys(Me)-Arg-Aib-Asn-Cys]-Tle-Thr-NH₂ (PSM-0410);
Ac-Thr-Aib-Hfe-[Cys-Lys-Arg-Aib-Asn-Cys]-Tle-Thr-NH₂ (PSM-0411);
Ac-Thr-Aib-Mmf-[Cys-Lys-Arg-Aib-Asn-Cys]-Tle-Thr-NH₂ (PSM-0412);
DOTA-PPAc-Aib-Phe-[Cys-Lys-Arg-Aib-Asn-Cys]-Tle-Thr-NH₂ (PSM-0413);
Ac-Thr-Ala-Amf-[Cys-Lys-Arg-Aib-Asn-Cys]-Tle-Thr-NH₂ (PSM-0414);
Ac-Thr-Aib-Phe-[Cys-Lys-Arg-Lys(DOTA-Cmp)-Asn-Cys]-Tle-Thr-NH₂ (PSM-0415);
DOTA-Cmp-Thr-Aib-Pcf-[Cys-Lys-Arg-Glu-Asn-Cys]-Tle-Thr-NH₂ (PSM-0416);
Ac-Thr-Aib-Phe-[Cys-Lys-Arg-Lys(DOTA-Pamb)-Asn-Cys]-Tle-Thr-NH₂ (PSM-0419);
DOTA-Cmp-Thr-Aib-Pcf-[Smc-Lys(Me)-Arg(Me)-Aib-Asn-Cys]-Tle-NH₂
(alternative: DOTA-Cmp-Thr-Aib-Pcf-[Cys-Lys(Me)-Arg(Me)-Aib-Asn-Smc]-Tle-NH₂) (PSM-0420);
Ac-Thr-Aib-Pcf-[Cys-Lys-Cit-Aib-Asn-Cys]-leu-Thr-NH₂ (PSM-0421);
Ac-Thr-Aib-Phe-[Cys-Tap-Arg-Ala-Asn-Cys]-Tle-Thr-Asp-NH₂ (PSM-0422);
Ac-Thr-Aib-Phe-[Cys-Lys-Gln-Lys(DOTA-Ttds)-Asn-Cys]-Tle-Thr-NH₂ (PSM-0423);
Ac-Aib-Pcf-[Cys-Lys-Arg-Aib-Asn-Cys]-Tle-NH₂ (PSM-0424);
Hex-Thr-Aib-Phe-[Cys-Lys-Arg-Aib-Asn-Cys]-Tle-Thr-Asp-Ttds-lys(DOTA)-NH₂ (PSM-0425);
Ac-Thr-Aib-Phe-[Cys-Lys-Arg-Aib-Asn-Cys]-Tle-Bal-NH₂ (PSM-0426);
nBuCAyl-Thr-Aib-Pcf-[Cys-Lys-Opy-Aib-Asn-Cys]-Tle-Thr-Cmp-lys(DOTA)-NH₂ (PSM-0427);
Ac-Thr-Aib-Phe-[Cys-Lys-Arg-Aib-Asn-Cys]-Tle-Thr-Cmp-lys(DOTA)-NH₂ (PSM-0428);
DOTA-Cmp-Thr-Aib-Mcf-[Cys-Lys-Cit-Aib-Asn-Cys]-Tle-Thr-NH₂ (PSM-0431);
DOTA-Pamb-Aib-Pcf-[Cys-Lys-Arg-Aib-Asn-Cys]-Tle-Bal-NH₂ (PSM-0432);
nBuCAyl-Thr-Aib-Phe-[Cys-Lys-Cit-Aib-Asn-Cys]-Tle-Thr-Cmp-lys(DOTA)-NH₂ (PSM-0434);
Ac-Thr-Aib-Phe-[Cys-Gln-Arg-Ala-Asn-Cys]-Tle-Thr-Asp-NH₂ (PSM-0435);
DOTA-Bal-Aib-Phe-[Cys-Lys-Arg-Aib-Asn-Cys]-Tle-Thr-NH₂ (PSM-0437);
Ac-Thr-Aib-Phe-[Cys-Aph-Arg-Ala-Asn-Cys]-Tle-Thr-Asp-NH₂ (PSM-0439);
Ac-Thr-Aib-Cys(Bzl)-[Cys-Lys-Arg-Aib-Asn-Cys]-Tle-Thr-NH₂ (PSM-0441);
Ac-Aib-Phe-[Cys-Lys-Arg-Aib-Asn-Cys]-Tle-Thr-Asp-NH₂ (PSM-0442);
Ac-Aib-Pcf-[Cys-Lys-Arg-Aib-Asn-Cys]-NH₂ (PSM-0443);
Iva-Aib-Pcf-[Cys-Lys-Arg-Aib-Asn-Cys]-Tle-Thr-lys(DOTA)-NH₂ (PSM-0444);
DOTA-Cmp-Thr-Aib-Pcf-[Cys-Lys-Arg-Aib-Asn-Cys]-Tle-Thr-NH₂ (PSM-0445);
DOTA-Pab-Thr-Aib-Phe-[Cys-Lys-Arg-Aib-Asn-Cys]-Tle-Thr-NH₂ (PSM-0448);
DOTA-Cmp-Thr-Aib-Phe-[Cys-Lys-Arg-Aib-Asn-Cys]-Tle-Bal-NH₂ (PSM-0449);
Iva-Aib-Phe-[Cys-Lys-Arg-Aib-Asn-Cys]-Tle-Thr-NH₂ (PSM-0450);
Ac-Thr-Aib-Phe-[Cys-Nmk-Arg-Aib-Asn-Cys]-Tle-Thr-NH₂ (PSM-0451);
nBuCAyl-Thr-Aib-Pcf-[Cys-Lys-Arg-Aib-Asn-Cys]-Tle-Thr-lys(DOTA-Cmp)-NH₂ (PSM-0452);
Ac-Thr-Aib-Phe-[Cys-Lys-Arg-Aib-Asn-Cys]-Tle-Leu-NH₂ (PSM-0453);
DOTA-Cmp-Thr-Aib-Ppa-[Cys-Lys-Cit-Aib-Asn-Cys]-Tle-Thr-NH₂ (PSM-0454);
DOTA-Cmp-Nmt-Ala-Pcf-[Cys-Lys-Arg-Aib-Asn-Cys]-Tle-Thr-NH₂ (PSM-0455);
DOTA-Cmp-Thr-Aib-Pcf-[Cys-Lys-Arg-Aib-Asn-Cys]-Tle-en(Me) (PSM-0456);
DOTA-Pamb-Aib-Phe-[Cys-Lys-Arg-Aib-Asn-Cys]-Tle-NH₂ (PSM-0458);
DOTA-Cmp-Thr-Aib-Mnf-[Cys-Lys(Me)-Arg(Me)-Aib-Asn-Cys]-Tle-NH₂ (PSM-0459);
Ac-Thr-Ala-Phe-[Cys-Lys-Arg-Pro-Asn-Cys]-Nle-Thr-Asp-NH₂ (PSM-0460);
Hib-Pcf-[Cys-Lys-Arg-Aib-Asn-Cys]-Tle-Thr-NH₂ (PSM-0461);
Hex-Thr-Aib-Phe-[Cys-Lys-Arg-Ala-Asn-Cys]-Tle-Thr-Ttds-lys(DOTA)-NH₂ (PSM-0462);
Ac-Thr-Aib-Phe-[Cys-Lys-Arg-Lys(DOTA-APAc)-Asn-Cys]-Tle-Thr-NH₂ (PSM-0464);
Ac-Thr-Aib-Phe-[Cys-Lys-Arg-Lys(DOTA-Ttds)-Asn-Cys]-Tle-Thr-NH₂ (PSM-0465);
DOTA-Cmp-Thr-Aib-Pcf-[Cys-Lys(Ac)-Arg-Aib-Asn-Cys]-Tle-Thr-NH₂ (PSM-0466);
DOTA-Cmp-Thr-Aib-Pcf-[Cys-Lys-Arg-Aib-Asn-Cys]-Tle-Bal-NH₂ (PSM-0467);
Ac-Ser-Ala-Phe-[Cys-Lys-Arg-Ala-Asn-Cys]-Nle-Thr-Asp-NH₂ (PSM-0469);
nBuCAyl-Thr-Aib-Mcf-[Cys-Lys-Arg-Aib-Asn-Cys]-Tle-Thr-Cmp-lys(DOTA)-NH₂ (PSM-0470);
Ac-Thr-Aib-Phe-[Cys-Lys-Arg-Aib-Asn-Cys]-Tle-Thr-Pab-lys(DOTA)-NH₂ (PSM-0471);
Ac-Aib-Pcf-[Cys-Lys(Me)-Arg-Aib-Asn-Cys]-Tle-en-DOTA (PSM-0472);
Ac-Thr-Ala-Phe-[Cys-Lys-Arg-Ala-Asn-Cys]-Nle-Ala-Asp-NH₂ (PSM-0476);
Ac-Thr-Aib-Phe-[Cys-Lys-Arg-Aib-Asn-Cys]-Tle-Gly-NH₂ (PSM-0477);
Ac-Thr-Aib-Phe-[Cys-Lys-Arg-Aib-Asp-Cys]-Tle-Thr-NH₂ (PSM-0478);
Ac-Thr-Aib-Phe-[Cys-Lys-Arg-Aib-Gly-Cys]-Tle-Thr-NH₂ (PSM-0479);
DOTA-Ttds-Thr-Ala-Phe-[Cys-Lys-Arg-Ala-Asn-Cys]-Leu-NH₂ (PSM-0480);
Ac-Aib-Pcf-[Cys-Lys(Me)-Arg(Me)-Aib-Asn-Cys]-Tle-en-DOTA (PSM-0481);
Ac-Thr-Aib-Pcf-[Cys-Lys-Arg-Aib-Asn-Cys]-Tle-Thr-Cmp-lys(DOTA)-NH₂ (PSM-0482);
DOTA-Cmp-Thr-Aib-Pcf-[Cys-KMe3-Arg(Me)-Aib-Asn-Cys]-Tle-Thr-NH₂ (PSM-0483);
nBuCAyl-Thr-Aib-Phe-[Cys-Lys-Arg-Aib-Asn-Cys]-Tle-Thr-Cmp-lys(DOTA)-NH₂ (PSM-0484);
DOTA-Cmp-Thr-Aib-Pcf-[Cys-Hgn-Arg-Aib-Asn-Cys]-Tle-Thr-NH₂ (PSM-0485);
Ac-Thr-Aib-Mcf-[Cys-Lys-Arg-Aib-Asn-Cys]-Tle-Thr-Cmp-lys(DOTA)-NH₂ (PSM-0486);
Ac-Thr-Aib-Tyr-[Cys-Lys-Arg-Aib-Asn-Cys]-Tle-Thr-Asp-NH₂ (PSM-0488);
DOTA-Cmp-Aib-Pcf-[Cys-Lys-Gln(Gu)-Aib-Asn-Cys]-Tle-Thr-NH₂ (PSM-0489);
DOTA-Cmp-Thr-Aib-Pcf-[Cys-Lys(Bio)-Arg-Aib-Asn-Cys]-Tle-Thr-NH₂ (PSM-0490);
Ac-Ala-Ala-Phe-[Cys-Lys-Arg-Ala-Asn-Cys]-Nle-Thr-Asp-NH₂ (PSM-0491);
Ac-Thr-Aib-Pcf-[Cys-Lys-Arg-Aib-Asn-Cys]-Tle-Thr-OH (PSM-0493);
Ac-Aib-Pcf-[Cys-Lys-Gln(Gu)-Aib-Asn-Cys]-Tle-Thr-Cmp-lys(DOTA)-NH₂ (PSM-0494);
Crown-Cmp-Thr-Aib-Pcf-[Cys-Lys(Me)-Arg(Me)-Aib-Asn-Cys]-Tle-Thr-NH₂ (PSM-0495);
Ac-Thr-Aib-Pcf-[Cys-Lys-Cit-Aib-Glu-Cys]-Tle-Thr-NH₂ (PSM-0496);
DOTA-Cmp-Thr-Aib-Pcf-[Cys-Lys-Arg-Aib-Asn-Cys]-Nml-Thr-NH₂ (PSM-0497);
H-Met-Thr-Ala-Phe-[Cys-Lys-Arg-Ala-Asn-Cys]-Leu-Thr-Asp-Gly-Ser-NH₂ (PSM-0498);
DOTA-Cmp-Thr-Aib-Pcf-[Cys-Lys(Me)-Egd-Aib-Asn-Cys]-Tle-Thr-NH₂ (PSM-0499);
nBuCAyl-Thr-Aib-Pcf-[Cys-Lys-Cit-Aib-Asn-Cys]-Tle-Thr-lys(DOTA-Cmp)-NH₂ (PSM-0500);
Ac-Thr-Aib-Phe-[Cys-Lys-Arg-Ala-Asn-Cys]-Nle-Thr-Asp-NH₂ (PSM-0501);
Hex-Thr-Aib-Phe-[Cys-Lys-Arg-Ala-Asn-Cys]-Tle-Thr-Asp-Ttds-lys(DOTA)-NH₂ (PSM-0502);
DOTA-Cmp-Thr-Aib-Mmf-[Cys-Lys(Me)-Arg(Me)-Aib-Asn-Cys]-Tle-NH₂ (PSM-0503);
DOTA-Ttds-Thr-Ala-Phe-[Cys-Lys-Arg-Ala-Asn-Cys]-Nle-Thr-Asp-NH₂ (PSM-0504);
DOTA-O2Oc-Thr-Aib-Phe-[Cys-Lys-Arg-Aib-Asn-Cys]-Tle-Thr-NH₂ (PSM-0505);
Ac-Aib-Phe-[Cys-Lys-Arg-Ala-Asn-Cys]-Tle-Thr-Asp-NH₂ (PSM-0506);
DOTA-Cmp-Aib-Phe-[Cys-Lys-Arg-Aib-Asn-Cys]-Tle-Thr-NH₂ (PSM-0507);
Ac-Thr-Pam-Phe-[Cys-Lys-Arg-Aib-Asn-Cys]-Tle-Thr-NH₂ (PSM-0508);
HPA-Aib-Pcf-[Cys-Lys-Arg-Aib-Asn-Cys]-Tle-Thr-Cmp-lys(DOTA)-NH₂ (PSM-0509);
Ac-Thr-Aib-Phe-[Cys-Lys-Arg-Aib-Asn-Cys]-Tle-NHBu (PSM-0510);
DOTA-Cmp-Thr-Aib-Pcf-[Cys-Orn-Arg-Aib-Asn-Cys]-Tle-Thr-NH₂ (PSM-0511);
DOTA-Cmp-Thr-Aib-Opa-[Cys-Lys-Cit-Aib-Asn-Cys]-Tle-Thr-NH₂ (PSM-0512);
DOTA-Ahx-Aib-Phe-[Cys-Lys-Arg-Aib-Asn-Cys]-Tle-Thr-NH₂ (PSM-0513);
Ac-Thr-Aib-Pcf-[Cys-Lys-Arg-Aib-Asn-Cys]-Tle-Bal-NH₂ (PSM-0514);
Macropa-Cmp-Thr-Aib-Pcf-[Cys-Lys(Me)-Arg(Me)-Aib-Asn-Cys]-Tle-Thr-NH₂ (PSM-0515);
Ac-Aib-Pcf-[Cys-Lys-Arg-Aib-Asn-Cys]-Tle-en-DOTA (PSM-0516);
Ac-Thr-Aib-2Ni-[Cys-Lys-Arg-Aib-Asn-Cys]-Tle-Thr-Asp-NH₂ (PSM-0517);
HYDAc-Aib-Phe-[Cys-Lys-Arg-Aib-Asn-Cys]-Tle-Thr-NH₂ (PSM-0518);
DOTA-Ttds-Thr-Aib-Phe-[Cys-Lys-Gln-Aib-Asn-Cys]-Tle-Thr-NH₂ (PSM-0521);
Ac-Thr-Aib-Phe-[Cys-Lys-Arg-Aib-Asn-Cys]-Tle-Thr-Pamb-lys(DOTA)-NH₂ (PSM-0522);
Ac-Thr-Aib-Phe-[Cys-Lys-Arg-Aib-Asn-Cys]-Tle-Thr-O2Oc-lys(DOTA)-NH₂ (PSM-0529);
HPA-Aib-Phe-[Cys-Lys-Arg-Aib-Asn-Cys]-Tle-Thr-NH₂ (PSM-0530);
DOTA-Cmp-Thr-Aib-Pcf-[Cys-Lys-Hgn-Aib-Asn-Cys]-Tle-Thr-NH₂ (PSM-0531);
Ac-Thr-Aib-Phe-[Cys-Lys-Arg-Dtc-Asn-Cys]-Tle-Thr-Asp-NH₂ (PSM-0532);
Ac-Thr-Ala-Phe-[Cys-Lys-Arg-Ala-Asn-Cys]-Nle-Thr-Asp-Ttds-lys(DOTA)-NH₂ (PSM-0533);
DOTA-Thr-Aib-Pcf-[Cys-Lys-Arg-Aib-Asn-Cys]-Tle-NH₂ (PSM-0534);
Ac-Thr-Aib-Phe-[Cys-lys-Arg-Ala-Asn-Cys]-Tle-Thr-Asp-NH₂ (PSM-0535);
DOTA-Cmp-Tle-Aib-Pcf-[Cys-Lys(Me)-Arg(Me)-Aib-Asn-Cys]-Tle-Thr-NH₂ (PSM-0538);
DOTA-Thr-Aib-Phe-[Cys-Lys-Arg-Aib-Asn-Cys]-Tle-Bal-NH₂ (PSM-0539);
Ac-Thr-Aib-Phe-[Cys-Lys-Arg-Aib-Asn-Cys]-Tle-Gln-NH₂ (PSM-0540);
Ac-Thr-Aib-Phe-[Cys-Lys-Arg-Aib-Phe-Cys]-Tle-Thr-NH₂ (PSM-0541);
Ac-Thr-Aib-Phe-[Cys-Lys-Arg-Lys(DOTA-Ttds)-Asn-Cys]-Tle-Thr-Asp-NH₂ (PSM-0542);
DOTA-APAc-Thr-Aib-Phe-[Cys-Lys-Arg-Aib-Asn-Cys]-Tle-Thr-NH₂ (PSM-0543);
Ac-Thr-Aib-Phe-[Cys-Lys-Arg-Aib-Leu-Cys]-Tle-Thr-NH₂ (PSM-0545);
DOTA-Cmp-Thr-Aib-Pcf-[Cys-Lys-Urr-Aib-Asn-Cys]-Tle-Thr-NH₂ (PSM-0546);
Ac-Thr-Pro-Phe-[Cys-Lys-Arg-Ala-Asn-Cys]-Nle-Thr-Asp-NH₂ (PSM-0547);
DOTA-Cmp-Thr-Aib-Pcf-[Cys-Nmk-Arg-Aib-Asn-Cys]-Tle-Thr-NH₂ (PSM-0548);
DOTA-Cmp-Thr-Aib-Ptf-[Cys-Lys(Me)-Arg(Me)-Aib-Asn-Cys]-Tle-NH₂ (PSM-0549);
Ac-Thr-Aib-Ptf-[Cys-Lys-Arg-Aib-Asn-Cys]-Tle-Thr-NH₂ (PSM-0550);
Ac-Thr-Aib-Mff-[Cys-Lys-Arg-Aib-Asn-Cys]-Tle-Thr-NH₂ (PSM-0551);
Ac-Thr-Aib-Phe-[Cys-Lys-Arg-Ala-Asn-Cys]-Tle-Thr-NH₂ (PSM-0552);
DOTA-Cmp-Thr-Aib-Mff-[Cys-Lys(Me)-Arg(Me)-Aib-Asn-Cys]-Tle-NH₂ (PSM-0553);
Ac-Thr-Aib-Pcf-[Cys-Lys-Arg-Aib-Asn-Cys]-Tle-Throl-OH (PSM-0554);
Ac-Thr-Aib-Mnf-[Cys-Lys-Arg-Aib-Asn-Cys]-Tle-Thr-NH₂ (PSM-0555);
Ac-Thr-Aib-Phe-[Cys-Lys-Arg-Aib-Asn-Cys]-Tle-Phe-NH₂ (PSM-0556);
Ac-Thr-Aib-Ocf-[Cys-Lys-Arg-Aib-Asn-Cys]-Tle-Thr-Asp-NH₂ (PSM-0558);
Ac-Aib-Pcf-[Cys-Lys-Arg-Aib-Asn-Cys]-Tle-Thr-NH₂ (PSM-0559);
Ac-Thr-Aib-Phe-[Cys-Lys-Arg-Aib-Asn-Cys]-Npg-NH₂ (PSM-0560);
Ac-Thr-Ala-Phe-[Cys-Lys-Arg-Ala-Asn-Cys]-Nle-Thr-Ala-NH₂ (PSM-0562);
Ac-Thr-Aib-Phe-[Cys-Lys-Arg-Aib-Glu-Cys]-Tle-Thr-NH₂ (PSM-0563);
Ac-Thr-Aib-Phe-[Cys-Lys-Arg-Ala-Asn-Cys]-Tle-Thr-Asp-NH₂ (PSM-0565);
Ac-Thr-Aib-Phe-[Smc-Lys-Arg-Ala-Asn-Cys]-Tle-Thr-Asp-NH₂
(alternative: Ac-Thr-Aib-Phe-[Cys-Lys-Arg-Ala-Asn-Smc]-Tle-Thr-Asp-NH₂) (PSM-0567);
Hex-Thr-Ala-Phe-[Cys-Lys-Arg-Ala-Asn-Cys]-Tle-Thr-Asp-Ttds-lys(DOTA)-NH₂ (PSM-0568);
Ac-Thr-Aib-Pcf-[Cys-Lys-Arg(Ac)-Aib-Asn-Cys]-Tle-Thr-NH₂ (PSM-0569);
Ac-Thr-Aib-Pcf-[Cys-Nle-Arg(Ac)-Aib-Asn-Cys]-Tle-Thr-NH₂ (PSM-0570);
nBuCAyl-Thr-Aib-Mcf-[Cys-Lys-Opy-Aib-Asn-Cys]-Tle-Thr-Cmp-lys(DOTA)-NH₂ (PSM-0571);
DOTA-Thr-Aib-Pcf-[Cys-Lys-Arg-Aib-Asn-Cys]-Tle-Thr-NH₂ (PSM-0573);
DOTA-Pamb-Aib-Phe-[Cys-Lys-Arg-Aib-Asn-Cys]-Tle-Bal-NH₂ (PSM-0574);
Bio-Ttds-Ttds-Thr-Aib-Pcf-[Cys-Lys-Arg-Aib-Asn-Cys]-Tle-Thr-NH₂ (PSM-0575);
Hex-Thr-Aib-Phe-[Cys-Lys-Arg-Aib-Asn-Cys]-Tle-Thr-Ttds-lys(DOTA)-NH₂ (PSM-0576);
HO-Succinyl-Aib-Phe-[Cys-Lys-Arg-Aib-Asn-Cys]-Tle-Thr-NH₂ (PSM-0577);
Ac-Thr-Ala-Phe-[Cys-Lys-Arg-Aib-Asn-Cys]-Nle-Thr-Asp-NH₂ (PSM-0578);
DOTA-Cmp-Thr-Aib-Pcf-[Cys-Lys-Arg(EtCAyl)-Aib-Asn-Cys]-Tle-Thr-NH₂ (PSM-0579);
DOTA-Thr-Aib-Phe-[Cys-Lys-Arg-Aib-Asn-Cys]-Tle-Thr-NH₂ (PSM-0580);
Ac-Thr-Aib-Phe-[Cys-Lys-Arg-Lys(DOTA-Ahx)-Asn-Cys]-Tle-Thr-NH₂ (PSM-0582);
DOTA-Cmp-Thr-Aib-Mcf-[Cys-Lys-Arg-Aib-Asn-Cys]-Tle-Thr-NH₂ (PSM-0583);
Ac-Thr-Aib-Pcf-[Cys-Lys-Cit-Aib-Leu-Cys]-Tle-Thr-NH₂ (PSM-0584);
DOTA-Cmp-Thr-Aib-Pcf-[Cys-Lys-Arg-Ala-Nmn-Cys]-Tle-Thr-NH₂ (PSM-0585);
Ac-Thr-Aib-Trp-[Cys-Lys-Arg-Aib-Asn-Cys]-Tle-Thr-Asp-NH₂ (PSM-0587);
Ac-Thr-Aib-Phe-[Cys-Lys-Arg-Aib-Trp-Cys]-Tle-Thr-NH₂ (PSM-0589);
DOTA-Cmp-Thr-Aib-Pcf-[Cys-Lys(Me)-RMe2a-Aib-Asn-Cys]-Tle-Thr-NH₂ (PSM-0590):
Macropa-Cmp-Thr-Aib-Pcf-[Cys-Lys(Me)-Arg(Me)-Aib-Asn-Cys]-Tle-Thr-NH2 (PSM-0591);
DOTA-Cmp-Thr-Aib-Pcf-[Cys-Lys(Me)-RMe1-Aib-Asn-Cys]-Tle-Thr-NH2 (PSM-0592);
DOTAM-Cmp-Thr-Aib-Pcf-[Cys-Lys(Me)-Arg(Me)-Aib-Asn-Cys]-Tle-Thr-NH2 (PSM-0593);
DOTA-Cmp-Thr-Aib-Pcf-[Cys-Lys(Me)-Arg(Me)-Aib-Asn-Cys]-OH (PSM-0594);
LSC-Cmp-Thr-Aib-Pcf-[Cys-Lys(Me)-Arg(Me)-Aib-Asn-Cys]-Tle-Thr-NH2 (PSM-0601);
DOTA-Cmp-Thr-Aib-Phe-[Cys-Lys(Me)-Arg(Me)-Aib-Asn-Cys]-Tle-en-H (PSM-0605)
and
DOTA-Cmp-Thr-Aib-Pcf-[Cys-Lys(Me)-Arg(Me)-Aib-Asn-Cys]-Tle-Bal-NH2(PSM-0606).

Embodiment 182. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 and 181, or pharmaceutically acceptable salt, solvate or hydrate thereof, wherein the compound is capable of binding to PSMA.

Embodiment 183. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181 and 182, or pharmaceutically acceptable salt, solvate or hydrate thereof, wherein the compound comprises a diagnostically active nuclide or a therapeutically active nuclide.

Embodiment 184. The compound of Embodiment 183, or pharmaceutically acceptable salt, solvate or hydrate thereof, wherein the compound comprises a diagnostically active nuclide.

Embodiment 185. The compound of Embodiment 184, or pharmaceutically acceptable salt, solvate or hydrate thereof, wherein the diagnostically active nuclide is a diagnostically active radionuclide.

Embodiment 186. The compound of Embodiment 185, or pharmaceutically acceptable salt, solvate or hydrate thereof, wherein the diagnostically active radionuclide is selected from the group consisting of ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I.

Embodiment 187. The compound of Embodiment 186, or pharmaceutically acceptable salt, solvate or hydrate thereof, wherein the diagnostically active radionuclide is selected from the group consisting of ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, and ²⁰³Pb.

Embodiment 188. The compound of Embodiment 187, or pharmaceutically acceptable salt, solvate or hydrate thereof, wherein the diagnostically active radionuclide is selected from the group consisting of ¹⁸F, ⁶⁴Cu, ⁶⁸Ga, and ¹¹¹In.

Embodiment 189. The compound of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 and 183, or pharmaceutically acceptable salt, solvate or hydrate thereof, wherein the compound comprises a therapeutically active nuclide.

Embodiment 190. The compound of Embodiment 189, or pharmaceutically acceptable salt, solvate or hydrate thereof, wherein the therapeutically active nuclide is a therapeutically active radionuclide.

Embodiment 191. The compound of Embodiment 190, or pharmaceutically acceptable salt, solvate or hydrate thereof, wherein the therapeutically active radionuclide is selected from the group consisting of ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, and ²¹¹At.

Embodiment 192. The compound of Embodiment 191, or pharmaceutically acceptable salt, solvate or hydrate thereof, wherein the therapeutically active radionuclide is selected from the group consisting of ⁴⁷Sc, ⁶⁷Cu, ⁹⁰Y, ¹⁶¹Tb, ¹⁷⁷Lu, ¹⁸⁸Re, ²¹²Pb, ²¹²Bi, ²¹³Bi, ²²⁵Ac, and ²²⁷Th.

Embodiment 193. The compound of Embodiment 192, or pharmaceutically acceptable salt, solvate or hydrate thereof, wherein the therapeutically active radionuclide is selected from the group consisting of ⁹⁰Y, ¹⁶¹Tb, ¹⁷⁷Lu, ²¹²Pb, ²²⁵Ac, and ²²⁷Th.

Embodiment 194. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187 and 188, for use in a method for the diagnosis of a disease.

Embodiment 195. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 189, 190, 191, 192 and 193, for use in a method for the treatment of a disease.

Embodiment 196. The compound or pharmaceutically acceptable salt, solvate or hydrate thereof for use of any one of Embodiments 194 and 195, wherein the disease is a disease involving the prostate specific membrane antigen (PSMA) protein.

Embodiment 197. The compound or pharmaceutically acceptable salt, solvate or hydrate thereof for use of any one of Embodiments 194, 195 and 196, wherein the disease involves cells showing upregulated expression of prostate specific membrane antigen (PSMA), preferably diseased tissue containing cells showing upregulated expression of PSMA.

Embodiment 198. The compound or pharmaceutically acceptable salt, solvate or hydrate thereof for use of any one of Embodiments 194, 195, 196 and 197, wherein the disease is a neoplasm, preferably a cancer or tumor.

Embodiment 199. The compound or pharmaceutically acceptable salt, solvate or hydrate thereof for use of Embodiment 198, wherein the tumor is selected from the group comprising an advanced tumor, a metastatic tumor, and a primary tumor.

Embodiment 200. The compound or pharmaceutically acceptable salt, solvate or hydrate thereof for use of any one of Embodiments 198 and 199, wherein the tumor is selected from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, a carcinoma, a squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), and combinations thereof.

Embodiment 201. The compound or pharmaceutically acceptable salt, solvate or hydrate thereof for use of Embodiment 200, wherein the tumor is a prostate tumor or a metastasized prostate tumor.

Embodiment 202. The compound or pharmaceutically acceptable salt, solvate or hydrate thereof for use of Embodiment 198, wherein the cancer, is selected from the group comprising: prostate cancer (e.g., metastatic castration resistant prostate cancer), renal cancer (e.g., clear cell carcinoma), head cancer, neck cancer, head and neck cancer, lung cancer (e.g., non-small cell lung cancer), salivary gland cancer, breast cancer, colorectal cancer, esophageal cancer, stomach cancer, liver cancer (e.g., hepatocellular cancer), thyroid cancer, glioblastoma, glioma, gall bladder cancer, laryngeal cancer, leukemia/lymphoma, uterine cancer, skin cancer (e.g., melanoma), endocrine cancer, sarcoma, urinary cancer, pancreatic cancer, gastrointestinal cancer, ovarian cancer, cervical cancer, endometrial cancer, fallopian tube cancer, primary peritoneal cancer, hematological cancer (e.g., diffuse large B cell lymphoma, Hodgkin's lymphoma, Non-Hodgkin's lymphoma, follicular lymphoma, acute myeloid leukemia, or multiple myeloma), cancer of unknown primary, adenomas, and tumor neovasculature.

Embodiment 203. The compound or pharmaceutically acceptable salt, solvate or hydrate thereof for use of Embodiment 202, wherein the cancer is prostate cancer.

Embodiment 204. The compound or pharmaceutically acceptable salt, solvate or hydrate thereof for use of any one of Embodiments 194, 196, 197, 198, 199, 200, 201, 202 and 203, wherein the compound comprises a diagnostically active nuclide, preferably a diagnostically active radionuclide.

Embodiment 205. The compound or pharmaceutically acceptable salt, solvate or hydrate thereof for use of Embodiment 204, wherein the diagnostically active nuclide is selected from the group comprising ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, ¹²⁵I, preferably ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, ²⁰³Pb, and more preferably ¹⁸F, ⁶⁴Cu, ⁶⁸Ga, and ¹¹¹In.

Embodiment 206. The compound or pharmaceutically acceptable salt, solvate or hydrate thereof for use of any one of Embodiments 194 and 196, 197, 198, 199, 200, 201, 202, 203, 204 and 205, wherein the method for the diagnosis is an imaging method.

Embodiment 207. The compound or pharmaceutically acceptable salt, solvate or hydrate thereof for use of Embodiment 206, wherein the imaging method is selected from the group consisting of scintigraphy, Single Photon Emission Computed Tomography (SPECT), Positron Emission Tomography (PET), SPECT/computed tomography, PET/computed tomography, and combinations thereof.

Embodiment 208. The compound or pharmaceutically acceptable salt, solvate or hydrate thereof for use of any one of Embodiments 194, 196, 197, 198, 199, 200, 201, 202, 203, 204, 205, 206 and 207, wherein the method comprises the administration of a diagnostically effective amount of the compound to a subject, preferably to a mammal, wherein the mammal is selected from the group comprising man, companion animals, pets, and livestock, more preferably the subject is selected from the group comprising man, dog, cat, horse, and cow, and most preferably the subject is a human being.

Embodiment 209. The compound or pharmaceutically acceptable salt, solvate or hydrate thereof for use of any one of Embodiments 195, 196, 197, 198, 199, 200, 201, 202 and 203, wherein the compound comprises a therapeutically active nuclide, preferably a therapeutically active radionuclide.

Embodiment 210. The compound or pharmaceutically acceptable salt, solvate or hydrate thereof for use of Embodiment 209, wherein the therapeutically active nuclide is selected from the group comprising ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, ²¹¹At, preferably ⁴⁷Sc, ⁶⁷Cu, ⁹⁰Y, ¹⁶¹Tb, ¹⁷⁷Lu, ¹⁸⁸Re, ²¹²Pb, ²¹²Bi, ²¹³Bi, ²²⁵Ac, ²²⁷Th, and more preferably ⁹⁰Y, ¹⁶¹Tb, ¹⁷⁷Lu, ²¹²Pb, ²²⁵Ac, and ²²⁷Th.

Embodiment 211. The compound or pharmaceutically acceptable salt, solvate or hydrate thereof for use of any one of Embodiments 195, 196, 197, 198, 199, 200, 201, 202, 203 209 and 210, wherein the method comprises the administration of a therapeutically effective amount of the compound to a subject, preferably to a mammal, wherein the mammal is selected from the group comprising man, companion animals, pets, and livestock, more preferably the subject is selected from the group comprising man, dog, cat, horse, and cow, and most preferably the subject is a human being.

Embodiment 212. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, for use in a method for the identification of a subject, wherein the subject is likely to respond or likely not to respond to a treatment of a disease, wherein the method for the identification of a subject comprises carrying out a method of diagnosis using the compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, preferably a method for the diagnosis of a disease as described in any one of Embodiments 194, 196, 197, 198, 199, 200, 201, 202, 203, 204, 205, 206, 207 and 208.

Embodiment 213. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, for use in a method for the selection of a subject from a group of subjects, wherein the subject is likely to respond or likely not to respond to a treatment of a disease, wherein the method for the selection of a subject from a group of subjects comprises carrying out a method of diagnosis using the compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114,115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, preferably a method for the diagnosis of a disease as described in any one of Embodiments 194, 196, 197, 198, 199, 200, 201, 202, 203, 204, 205, 206, 207 and 208.

Embodiment 214. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosis using the compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 204 and 205, preferably a method for the diagnosis of a disease as described in any one of Embodiments 194, 196, 197, 198, 199, 200, 201, 202, 203, 204, 205, 206, 207 and 208.

Embodiment 215. The compound or pharmaceutically acceptable salt, solvate or hydrate thereof for use of any one of Embodiments 212, 213 and 214, wherein the disease is a disease involving the prostate specific membrane antigen (PSMA) protein.

Embodiment 216. The compound or pharmaceutically acceptable salt, solvate or hydrate thereof for use of any one of Embodiments 212, 213, 214 and 215, wherein the disease involves cells showing upregulated expression of prostate specific membrane antigen (PSMA).

Embodiment 217. The compound or pharmaceutically acceptable salt, solvate or hydrate thereof for use of any one of Embodiments 212, 213, 214, 215 and 216, wherein the disease is a neoplasm, preferably a cancer or tumor.

Embodiment 218. The compound or pharmaceutically acceptable salt, solvate or hydrate thereof for use of Embodiment 217, wherein the tumor is selected from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, a carcinoma, a squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), and combinations thereof.

Embodiment 219. The compound or pharmaceutically acceptable salt, solvate or hydrate thereof for use of Embodiment 218, wherein the tumor is a prostate tumor or a metastasized prostate tumor.

Embodiment 220. The compound or pharmaceutically acceptable salt, solvate or hydrate thereof for use of Embodiment 217, wherein the neoplasm, cancer, and tumor are each and individually selected from the group comprising prostate cancer (e.g., metastatic castration resistant prostate cancer), renal cancer (e.g., clear cell carcinoma), head cancer, neck cancer, head and neck cancer, lung cancer (e.g., non-small cell lung cancer), salivary gland cancer, breast cancer, colorectal cancer, esophageal cancer, stomach cancer, liver cancer (e.g., hepatocellular cancer), thyroid cancer, glioblastoma, glioma, gall bladder cancer, laryngeal cancer, leukemia/lymphoma, uterine cancer, skin cancer (e.g., melanoma), endocrine cancer, sarcoma, urinary cancer, pancreatic cancer, gastrointestinal cancer, ovarian cancer, cervical cancer, endometrial cancer, fallopian tube cancer, primary peritoneal cancer, hematological cancer (e.g., diffuse large B cell lymphoma, Hodgkin's lymphoma, Non-Hodgkin's lymphoma, follicular lymphoma, acute myeloid leukemia, or multiple myeloma), cancer of unknown primary, adenomas, and tumor neovasculature.

Embodiment 221. The compound or pharmaceutically acceptable salt, solvate or hydrate thereof for use of Embodiment 220, wherein the cancer is prostate cancer.

Embodiment 222. The compound or pharmaceutically acceptable salt, solvate or hydrate thereof for use of any one of Embodiments 212, 213, 214, 215, 216, 217, 218, 219, 220 and 221, wherein the method of diagnosis is an imaging method.

Embodiment 223. The compound or pharmaceutically acceptable salt, solvate or hydrate thereof for use of Embodiment 222 wherein the imaging method is selected from the group comprising scintigraphy, Single Photon Emission Computed Tomography (SPECT), Positron Emission Tomography (PET), SPECT/computed tomography, PET/computed tomography, and combinations thereof, and combinations thereof.

Embodiment 224. The compound or pharmaceutically acceptable salt, solvate or hydrate thereof for use of any one of Embodiments 212, 213, 214, 215, 216, 217, 218, 219, 220 and 221, wherein the compound comprises a diagnostically active nuclide, preferably a diagnostically active radionuclide.

Embodiment 225. The compound or pharmaceutically acceptable salt, solvate or hydrate thereof for use of Embodiment 224, wherein the diagnostically active nuclide is selected from the group comprising ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I, preferably ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, and ²⁰³Pb, and more preferably ¹⁸F, ⁶⁴Cu, ⁶⁸Ga, and ¹¹¹In.

Embodiment 226. The compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192 and 193, for use in a method for delivering a diagnostically active radionuclide or a therapeutically active radionuclide to prostate specific membrane antigen (PSMA).

Embodiment 227. The compound or pharmaceutically acceptable salt, solvate or hydrate thereof for use of Embodiment 226, wherein the diagnostically active radionuclide is selected from the group consisting of ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I, preferably ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, and ²⁰³Pb, and more preferably ¹⁸F, ⁶⁴Cu, ⁶⁸Ga, and ¹¹¹In.

Embodiment 228. The compound or pharmaceutically acceptable salt, solvate or hydrate thereof for use of Embodiment 226, wherein the therapeutically active radionuclide is selected from the group consisting of ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, ²¹¹At, preferably ⁴⁷Sc, ⁶⁷Cu, ⁹⁰Y, ¹⁶¹Tb, ¹⁷⁷Lu, ¹⁸⁸Re, ²¹²Pb, ²¹²Bi, ²¹³Bi, ²²⁵Ac, ²²⁷Th, and more preferably ⁹⁰Y, ¹⁶¹Tb, ¹⁷⁷Lu, ²¹²Pb, ²²⁵Ac, and ²²⁷Th.

Embodiment 229. The compound or pharmaceutically acceptable salt, solvate or hydrate thereof for use of any one of Embodiments 226-228, wherein the prostate specific membrane antigen (PSMA) is expressed by a cell, preferably a prostate cell, a metastasized prostate cell, a lung cell, a renal cell, a pancreatic cell, a bladder cell, a breast cell, a colon cell, a germ cell, an esophageal cell, a stomach cell, an endothelial cell and combinations thereof each showing upregulated expression of PSMA.

Embodiment 230. The compound or pharmaceutically acceptable salt, solvate or hydrate thereof for use of Embodiment 229, wherein the cell is contained in or part of a tissue, preferably a diseased tissue of a subject suffering from a disease.

Embodiment 231. The compound or pharmaceutically acceptable salt, solvate or hydrate thereof for use of Embodiment 230, wherein the disease involves cells showing upregulated expression of PSMA, preferably diseased tissue containing cells showing upregulated expression of PSMA.

Embodiment 232. The compound or pharmaceutically acceptable salt, solvate or hydrate thereof for use of any one of Embodiments 230 and 231, wherein the disease is a neoplasm, preferably a cancer or tumor.

Embodiment 233. The compound or pharmaceutically acceptable salt, solvate or hydrate thereof for use of Embodiment 232, wherein the tumor is selected from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, a carcinoma, a squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), and combinations thereof.

Embodiment 234. The compound or pharmaceutically acceptable salt, solvate or hydrate thereof for use of Embodiment 232, wherein the cancer is selected from the group comprising prostate cancer (e.g., metastatic castration resistant prostate cancer), renal cancer (e.g., clear cell carcinoma), head cancer, neck cancer, head and neck cancer, lung cancer (e.g., non-small cell lung cancer), salivary gland cancer, breast cancer, colorectal cancer, esophageal cancer, stomach cancer, liver cancer (e.g., hepatocellular cancer), thyroid cancer, glioblastoma, glioma, gall bladder cancer, laryngeal cancer, leukemia/lymphoma, uterine cancer, skin cancer (e.g., melanoma), endocrine cancer, sarcoma, urinary cancer, pancreatic cancer, gastrointestinal cancer, ovarian cancer, cervical cancer, endometrial cancer, fallopian tube cancer, primary peritoneal cancer, hematological cancer (e.g., diffuse large B cell lymphoma, Hodgkin's lymphoma, Non-Hodgkin's lymphoma, follicular lymphoma, acute myeloid leukemia, or multiple myeloma), cancer of unknown primary, adenomas, and tumor neovasculature.

Embodiment 235. The compound or pharmaceutically acceptable salt, solvate or hydrate thereof of any one of Embodiments 186-188, 205, 225, and 227, wherein the diagnostically active nuclide is ¹⁸F, wherein the diagnostically active nuclide is bound to aluminium, wherein the aluminium is bound to the chelator and bound to ¹⁸F.

Embodiment 236. A composition, preferably a pharmaceutical composition, wherein the composition comprises a compound or pharmaceutically acceptable salt, solvate or hydrate thereof according to any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 205, 210, 224, 225, 227 and 228, and a pharmaceutically acceptable excipient.

Embodiment 237. The composition of Embodiment 236 for use in any method as defined in any of the preceding embodiments.

Embodiment 238. A method for the diagnosis of a disease in a subject, wherein the method comprises administering to the subject a diagnostically effective amount of a compound or pharmaceutically acceptable salt, solvate or hydrate thereof according to any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187 and 188.

Embodiment 239. The method of Embodiment 238, wherein the compound or pharmaceutically acceptable salt, solvate or hydrate thereof comprises a diagnostically active nuclide, whereby the nuclide is preferably a diagnostically active radionuclide.

Embodiment 240. A method for the treatment of a disease in a subject, wherein the method comprises administering to the subject a therapeutically effective amount of a compound or pharmaceutically acceptable salt, solvate or hydrate thereof according to any one of Embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180,181, 189, 190, 191, 192 and 193.

Embodiment 241. The method of Embodiment 240, wherein the compound or pharmaceutically acceptable salt, solvate or hydrate thereof comprises a therapeutically active nuclide, whereby the nuclide is preferably a therapeutically active radionuclide.

Embodiment 242. The method of any one of Embodiments 238, 239, 240 and 241, wherein the disease is a disease involving the prostate specific membrane antigen (PSMA) protein.

Embodiment 243. The method of any one of Embodiments 238, 239, 240, 241 and 242, wherein the disease involves cells showing upregulated expression of prostate specific membrane antigen (PSMA), preferably diseased tissue containing cells showing upregulated expression of PSMA.

Embodiment 244. A kit comprising a compound or pharmaceutically acceptable salt, solvate or hydrate thereof according to any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 205, 210, 224, 225, 227 and 228, one or more optional excipient(s) and optionally one or more device(s), whereby the device(s) is/are selected from the group comprising a labeling device, a purification device, a handling device, a radioprotection device, an analytical device or an administration device.

Embodiment 245. The kit of Embodiment 244 for use in any method as defined in any of the preceding Embodiments.

Embodiment 246. Use of compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194 and 195 in or for the manufacture of a medicament, preferably a medicament for the treatment of a disease as disclosed herein.

Embodiment 247. Use of compound or pharmaceutically acceptable salt, solvate or hydrate thereof of any one of Embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194 and 195 in or for the manufacture of a diagnostic means, preferably a diagnostic means for the diagnosis of a disease as disclosed herein.

It will be acknowledged by a person skilled in the art that a compound of the disclosure is any compound disclosed herein, including but not limited to any compound described in any of the above embodiments and any of the following embodiments.

It will be acknowledged by a person skilled in the art that a method of the disclosure is any method disclosed herein, including but not limited to any method described in any of the above embodiments and any of the following embodiments.

It will be acknowledged by a person skilled in the art that a composition of the disclosure is any composition disclosed herein, including but not limited to any composition described in any of the above embodiments and any of the following embodiments.

It will be acknowledged by a person skilled in the art that a kit of the disclosure is any kit disclosed herein, including but not limited to any kit described in any of the above embodiments and any of the following embodiments.

It will be acknowledged by a person skilled in the art that the expression “aspect of the disclosure” is used synonymously with the term “aspect of the invention” and, respectively, “aspect of the present invention”, and that the expression “embodiment of the disclosure” is used synonymously with the term “embodiment of the invention” and, respectively, “embodiment of the present invention”.

Except where otherwise indicated, all numbers expressing quantities of amounts, conditions, and so forth used in the disclosure are to be understood as being modified in all instances by the term “about.” Accordingly, unless indicated to the contrary, the numerical parameters set forth in the present disclosure are approximations that may vary depending upon the desired properties sought to be obtained by the present disclosure. At the very least, and not to be considered as an attempt to limit the application of the doctrine of equivalents, each numerical parameter should be construed in light of the number of significant digits and ordinary rounding conventions.

Additionally, the disclosure of numerical ranges within the present disclosure is considered to be a disclosure of all numerical values and ranges within that range. For example, if a range is from 1 to 10, it is deemed to include, for example, 1, 2, 2.2, 3, 4, 5, 6, 7, 7.4, 7.6, 8, 8.7, 9, 9.5, 10, or any other value or range (integer or non-integer) within the range. Moreover, as used herein, the term “at least” includes the stated number, e.g., “at least 50” includes 50.

The expression alkyl as preferably used herein refers each and individually to a saturated, straight-chain or branched hydrocarbon group and is usually accompanied by a qualifier which specifies the number of carbon atoms it may contain. For example, the expression (C₁-C₆)alkyl means each and individually any of methyl, ethyl, n-propyl, isopropyl, n-butyl, isobutyl, sec-butyl, tert-butyl, n-pentyl, 1-methyl-butyl, 1-ethyl-propyl, 3-methyl-butyl, 1,2-dimethyl-propyl, 2-methyl-butyl, 1,1-dimethyl-propyl, 2,2-dimethylpropyl, n-hexyl, 1,1-dimethyl-butyl and any other isoform of alkyl groups containing six saturated carbon atoms.

In an embodiment and as preferably used herein, “(C₁-C₂)alkyl” means each and individually any of methyl and ethyl.

In an embodiment and as preferably used herein, “(C₁-C₃)alkyl” means each and individually any of methyl, ethyl, n-propyl and isopropyl.

In an embodiment and as preferably used herein, “(C₁-C₄)alkyl” means each and individually any of methyl, ethyl, n-propyl, isopropyl, n-butyl, isobutyl, sec-butyl and tert-butyl.

In an embodiment and as preferably used herein, “(C₁-C₆)alkyl” means each and individually any of methyl, ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl and 3,3-dimethyl-but-2-yl.

In an embodiment, and as preferably used herein, “(C₁-C₈)alkyl” refers to a saturated or unsaturated, straight-chain or branched hydrocarbon group having from 1 to 8 carbon atoms. Representative (C₁-C₈)alkyl groups include, but are not limited to, any of methyl, ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl, 3,3-dimethyl-but-2-yl, n-heptyl, 2-heptyl, 2-methyl-hexyl, 3-methyl-hexyl, 4-methyl-hexyl, 5-methyl-hexyl, 3-heptyl, 2-ethyl-pentyl, 3-ethyl-pentyl, 4-heptyl, 2-methyl-hex-2-yl, 2,2-dimethyl-pentyl, 3,3-dimethyl-pentyl, 4,4-dimethyl-pentyl, 3-methyl-hex-2-yl, 4-methyl-hex-2-yl, 5-methyl-hex-2-yl, 2,3-dimethyl-pentyl, 2,4-dimethyl-pentyl, 3,4-dimethyl-pentyl, 3-methyl-hex-3-yl, 2-ethyl-2-methyl-butyl, 4-methyl-hex-3-yl, 5-methyl-hex-3-yl, 2-ethyl-3-methyl-butyl, 2,3-dimethyl-pent-2-yl, 2,4-dimethyl-pent-2-yl, 3,3-dimethyl-pent-2-yl, 4,4-dimethyl-pent-2-yl, 2,2,3-trimethyl-butyl, 2,3,3-trimethyl-butyl, 2,3,3-trimethyl-but-2-yl, n-octyl, 2-octyl, 2-methyl-heptyl, 3-methyl-heptyl, 4-methyl-heptyl, 5-methyl-heptyl, 6-methyl-heptyl, 3-octyl, 2-ethyl-hexyl, 3-ethyl-hexyl, 4-ethyl-hexyl, 4-octyl, 2-propyl-pentyl, 2-methyl-hept-2-yl, 2,2-dimethyl-hexyl, 3,3-dimethyl-hexyl, 4,4-dimethyl-hexyl, 5,5-dimethyl-hexyl, 3-methyl-hept-2-yl, 4-methyl-hept-2-yl, 5-methyl-hept-2-yl, 6-methyl-hept-2-yl, 2,3-dimethyl-hex-1-yl, 2,4-dimethyl-hex-1-yl, 2,5-dimethyl-hex-1-yl, 3,4-dimethyl-hex-1-yl, 3,5-dimethyl-hex-1-yl, 3,5-dimethyl-hex-1-yl, 3-methyl-hept-3-yl, 2-ethyl-2-methyl-1-yl, 3-ethyl-3-methyl-1-yl, 4-methyl-hept-3-yl, 5-methyl-hept-3-yl, 6-methyl-hept-3-yl, 2-ethyl-3-methyl-pentyl, 2-ethyl-4-methyl-pentyl, 3-ethyl-4-methyl-pentyl, 2,3-dimethyl-hex-2-yl, 2,4-dimethyl-hex-2-yl, 2,5-dimethyl-hex-2-yl, 3,3-dimethyl-hex-2-yl, 3,4-dimethyl-hex-2-yl, 3,5-dimethyl-hex-2-yl, 4,4-dimethyl-hex-2-yl, 4,5-dimethyl-hex-2-yl, 5,5-dimethyl-hex-2-yl, 2,2,3-trimethyl-pentyl, 2,2,4-trimethyl-pentyl, 2,3,3-trimethyl-pentyl, 2,3,4-trimethyl-pentyl, 2,4,4-trimethyl-pentyl, 3,3,4-trimethyl-pentyl, 3,4,4-trimethyl-pentyl, 2,3,3-trimethyl-pent-2-yl, 2,3,4-trimethyl-pent-2-yl, 2,4,4-trimethyl-pent-2-yl, 3,4,4-trimethyl-pent-2-yl, 2,2,3,3-tetramethyl-butyl, 3,4-dimethyl-hex-3-yl, 3,5-dimethyl-hex-3-yl, 4,4-dimethyl-hex-3-yl, 4,5-dimethyl-hex-3-yl, 5,5-dimethyl-hex-3-yl, 3-ethyl-3-methyl-pent-2-yl, 3-ethyl-4-methyl-pent-2-yl, 3-ethyl-hex-3-yl, 2,2-diethyl-butyl, 3-ethyl-3-methyl-pentyl, 4-ethyl-hex-3-yl, 5-methyl-hept-3-yl, 2-ethyl-3-methyl-pentyl, 4-methyl-hept-4-yl, 3-methyl-hept-4-yl, 2-methyl-hept-4-yl, 3-ethyl-hex-2-yl, 2-ethyl-2-methyl-pentyl, 2-isopropyl-pentyl, 2,2-dimethyl-hex-3-yl, 2,2,4-trimethyl-pent-3-yl and 2-ethyl-3-methyl-pentyl. A (C₁-C₈)alkyl group can be unsubstituted or substituted with one or more groups, including, but not limited to, (C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment and as preferably used herein, “(C₂-C₆)alkyl” means each and individually any of ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl and 3,3-dimethyl-but-2-yl.

In an embodiment and as preferably used herein, “(C₄-C₆)alkyl” means each and individually any of n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl and 3,3-dimethyl-but-2-yl.

In an embodiment, and as preferably used herein, “carbocycle” refers to a saturated, unsaturated or aromatic mono- or bicyclic carbocyclic ring. A carbocycle can be unsubstituted or substituted with one or more groups, including, but not limited to, (C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment, and as preferably used herein, “heterocycle” refers to a saturated, unsaturated or aromatic mono- or bicyclic heterocyclic ring. A heterocycle group can be unsubstituted or substituted with one or more groups, including, but not limited to, (C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment, and as preferably used herein, “aryl” refers to a carbocyclic aromatic group. Examples of aryl groups include, but are not limited to, phenyl, naphthyl, and anthracenyl.

In an embodiment, and as preferably used herein, “heteroaryl” refers to a heterocyclic aromatic group. Examples of heteroaryl groups include, but are not limited to, furane, thiophene, pyridine, pyrimidine, benzothiophene, benzofurane, and quinoline.

In an embodiment, and as preferably used herein, “(C₅-C₆)heteroaryl” refers to a heterocyclic aromatic group consisting of 5 or 6 ring atoms wherein at least one atom is different from carbon, including, for example, nitrogen, sulfur or oxygen. A heterocyclic aromatic group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment, and as preferably used herein, “amino acid residue” refers to all atoms of an amino acid, which remain after the combination of said amino acid with other amino acids in a peptide chain.

In an embodiment, and as preferably used herein, “side chain” refers to all atoms of an amino acid residue that are not comprised in the “main chain” portion of said amino acid residue. “Main chain” refers to the structure that is formed by the consecutive connection of amino acids, whereby the α-nitrogen atom of an α-amino acid, the β-nitrogen atom of a β-amino acid, the γ-nitrogen of a γ-amino acid residue, the δ-nitrogen atom of a δ-amino acid, the ε-nitrogen of an ε-amino acid or the ω-nitrogen of an ω-amino acid is connected to the C-1 carbonyl atom of the preceeding amino acid.

In an embodiment, and as preferably used herein, atoms with unspecified atomic mass numbers in any structural formula or in any passage of the instant specification are either of unspecified isotopic composition, naturally occurring mixtures of isotopes or individual isotopes. This applies in particular to carbon, oxygen, nitrogen, sulfur, phosphorus, halogens and metal atoms, including but not limited to C, O, N, S, F, P, Cl, Br, At, Sc, Cr, Mn, Co, Fe, Cu, Ga, Sr, Zr, Y, Mo, Tc, Ru, Rh, Pd, Pt, Ag, In, Sb, Sn, Te, I, Pr, Pm, Dy, Sm, Gd, Tb, Ho, Dy, Er, Yb, Tm, Lu, Sn, Re, Rd, Os, Ir, Au, Pb, Bi, Po, Fr, Ra, Ac, Th, and Fm.

In an embodiment, and as preferably used herein, a “chelator” is a compound, which is capable of forming a chelate, whereby a chelate is a compound, including, for example, a cyclic compound where a metal or a moiety having an electron gap or a lone pair of electrons participates in the formation of the ring. In certain embodiments, a chelator is this kind of compound where a single ligand occupies more than one coordination site at a central atom.

In an embodiment, and as preferably used herein, a “diagnostically active compound” is a compound which is suitable for or useful in at least the diagnosis of a disease.

In an embodiment, and as preferably used herein, a “diagnostic agent” or a “diagnostically active agent” is a compound, which is suitable for or useful in at least the diagnosis of a disease.

In an embodiment, and as preferably used herein, a “diagnostically active radionuclide” is a radionuclide, which is suitable for or useful in at least the diagnosis of a disease. It will, however, also be acknowledged by a person skilled in the art that the use of said diagnostically active radionuclide may not be limited to diagnostic purposes, but can encompass their use in therapy and theragnostics.

In an embodiment, and as preferably used herein, a “therapeutically active compound” is a compound, which is suitable for or useful in at least the treatment of a disease.

In an embodiment, and as preferably used herein, a “therapeutic agent” or a “therapeutically active agent” is a compound which is suitable for or useful in at least the treatment of a disease.

In an embodiment, and as preferably used herein, a “therapeutically active radionuclide” is a radionuclide which is suitable for or useful in at least the treatment of a disease. It will, however, also be acknowledged by a person skilled in the art that the use of said therapeutically active radionuclide may not be limited to therapeutically purposes, but can encompass their use in diagnosis and theragnostics.

In an embodiment, and as preferably used herein, a “theragnostically active compound” is a compound, which is suitable for or useful in both the diagnosis and therapy of a disease.

In an embodiment, and as preferably used herein, a “theragnostic agent” or a “theragnostically active agent” is a compound which is suitable for or useful in both the diagnosis and therapy of a disease.

In an embodiment, and as preferably used herein, a “theragnostically active radionuclide” is a radionuclide, which is suitable for or useful in both the diagnosis and therapy of a disease.

In an embodiment, and as preferably used herein, “theragnostics” is a method for the combined diagnosis and therapy of a disease. In certain embodiments, the combined diagnostically and therapeutically active compounds used in theragnostics are radiolabeled.

In an embodiment, and as preferably used herein, “treatment of a disease” is treatment and/or prevention of a disease.

In an embodiment, and as preferably used herein, the terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

In an embodiment, and as preferably used herein, “preventing” or “prevent” describes reducing or eliminating the onset of the symptoms or complications of the disease, condition or disorder.

In an embodiment, and as preferably used herein, the term “subject” or “patient” includes a mammal. The mammal can be, e.g., any mammal, e.g., a human, companion animal, pet, livestock, dog, cat, horse, and cow.

In an embodiment, and as preferably used herein, a “disease involving the prostate specific membrane antigen (PSMA) protein” is a disease involving cells showing upregulated expression of PSMA, which are a or the cause for the disease and/or the symptoms of the disease, or are part of the pathology underlying the disease.

In an embodiment, and as preferably used herein, a “target cell” or “target tissue” is a cell or tissue, which is expressing prostate specific membrane antigen (PSMA) and is a or the cause for a disease and/or the symptoms of a disease, or is part of the pathology underlying a disease.

In an embodiment, and as preferably used herein, a “non-target cell” or “non-target tissue” is a cell or tissue, which is either not expressing prostate specific membrane antigen (PSMA) and/or is not a or the cause for a disease and/or the symptoms of a disease, or is part of the pathology underlying a disease.

In an embodiment, and as preferably used herein, a “neoplasm” is an abnormal new growth of cells. The cells in a neoplasm grow more rapidly than normal cells and will continue to grow if not treated. A neoplasm may be benign or malignant.

In an embodiment, and as preferably used herein, a “tumor” is a mass lesion that may be benign or malignant.

In an embodiment, and as preferably used herein, a “cancer” is a malignant neoplasm.

In an embodiment, and as preferably used herein, a “pharmaceutically acceptable excipient” refers to an ingredient other than the active agent(s) and/or compound(s) that is suitable for use in a pharmaceutical composition, including, but not limited to, pharmaceutically acceptable adjuvants, diluents, carriers, buffers, binders, colorants, lubricants, fillers, disintegrants, preservatives, surfactants, and stabilizers.

In an embodiment, the compounds disclosed and the compounds subject to the embodiments disclosed herein encompass a pharmaceutically acceptable salt of such compounds, a solvate of such compounds or a hydrate of such compounds.

In an embodiment, and as preferably used herein, a “linkage” is an attachment of two atoms of two independent moieties. A preferred linkage is a chemical bond or a plurality of chemical bonds. Preferably a chemical bond is a covalent bond or a plurality of chemical bonds.

Preferably the linkage is a covalent bond or a coordinate bond. As preferably used herein, an embodiment of a coordinate bond is a bond or group of bonds as realized when a metal is bound by a chelator. Depending on the type of atoms linked and their atomic environment different types of linkages are created. These types of linkage are defined by the type of atom arrangements created by the linkage. For instance, the linking of a moiety comprising an amine with a moiety comprising a carboxylic acid leads to a linkage named amide (which is also referred to as amide linkage, —CO—N—, —N—CO—). It will be acknowledged by a person skilled in the art that this and the following examples of creating linkages are only prototypical examples and are by no means limiting the scope of the instant application. It will be acknowledged by a person in the art that the linking of a moiety comprising an isothiocyanate with a moiety comprising an amine leads to thiourea (which is also referred to as a thiourea linkage, —N—CS—N—), and linking of a moiety comprising a C atom with a moiety comprising a thiol-group (—C—SH) leads to thioether (which is also referred to as a thioether linkage, —C—S—C). A non-limiting list of examples of linkages used in connection with the chelator and the rest of the compound of the disclosure and their characteristic type of atom arrangement is presented Table 1.

TABLE 1

| Linkage | Characteristic atom arrangement |
|---|---|
| Amide | 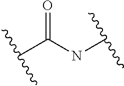 [SMILES: *NC(*)=O] |
| Sulfonamide | 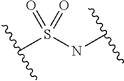 [SMILES: *NS(*)(=O)=O] |
| Urea | 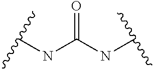 [SMILES: *NC(=O)N*] |
| Thioether | 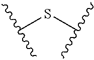 [SMILES: *S*] |
| Disulfide | 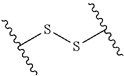 [SMILES: *SS*] |
| Carbamate | 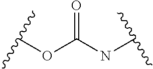 [SMILES: *NC(=O)O*] |
| Thiourea | 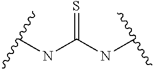 [SMILES: *NC(=S)N*] |
| Triazole | 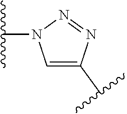 [SMILES: *c1cn(*)nn1] |

Examples of reactive groups which, in some embodiments of the disclosure, are used in the formation of linkages between the chelator and the rest of the compound of the disclosure are summarized in Table 2. It will, however, be understood by a person skilled in the art that neither the linkages nor the reactive groups forming such linkages for the formation of the compounds of the disclosure are limited to the ones of Table 2.

TABLE 2

| first reactive group | second reactive group | (type of) linkage |
|---|---|---|
| Amino | Carboxylic acid | Amide |
| Amino | Activated carboxylic acid | Amide |
| Carboxylic acid | Amino | Amide |
| Sulfhydryl | Michael acceptor | Thioether |
| | (e.g., Maleimide) | |
| Bromo | Sulfhydryl | Thioether |
| Isothiocyanate | Amino | Thiourea |
| Azide | Alkyne | Triazole |
| Isocyanate | Amino | Carbamate |

In an embodiment, and as preferably used herein, the term “activated carboxylic acid” refers to a carboxylic acid group with the general formula —CO—X, wherein X is a leaving group. For example, activated forms of a carboxylic acid group may include, but are not limited to, acyl chlorides, symmetrical or unsymmetrical anhydrides, and esters. In some embodiments, the activated carboxylic acid group is an ester with pentafluorophenol, nitrophenol, benzotriazole, azabenzotriazole, thiophenol, ethyl 2-cyano-2-(hydroxyimino)acetate or N-hydroxysuccinimide (NHS) as leaving group.

In an embodiment, and as preferably used herein, the term “mediating a linkage” means that a linkage or a type of linkage is established, preferably a linkage between two moieties.

Compounds of the disclosure may contain amino acid sequences as provided herein. Conventional amino acids, also referred to as natural amino acids are identified according to their standard three-letter codes and one-letter abbreviations, as set forth in Table 3.

TABLE 3
Conventional amino acids and their abbreviations

| | 3-letter | 1-letter |
|---|---|---|
| Amino acid | abbreviation | abbreviation |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Non-conventional amino acids, also referred to as non-natural amino acids, are any kind of non-oligomeric compound which comprises an amino group and a carboxylic group and is not a conventional amino acid.

Examples of non-conventional amino acids and other building blocks as used for the construction compounds of the invention are identified according to their abbreviation or name found in Table 4. The structures of some building blocks are depicted with an exemplary reagent for introducing the building block into the peptide (e.g., as carboxylic acid like) or these building blocks are shown as residue which is completely attached to another structure like a peptide or amino acid. The structures of the amino acids are shown as explicit amino acids and not as residues of the amino acids how they are presented after implementation in the peptide sequence. Some larger chemical moieties consisting of more than one moiety are also shown for the reason of clarity.

TABLE 4
Abbreviation, name and structure of non-natural amino-acid and other building
blocks and chemical moieties

| Abbreviation | Name | Residue | Building Block |
|---|---|---|---|
| 1Ni | 3-(1-naphthyl)alanine | 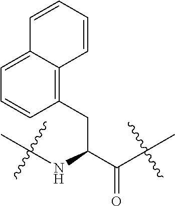 [SMILES: *N[C@@H](Cc1cccc2ccccc12)C(*)=O] | 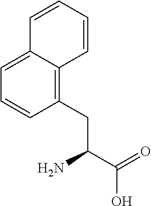 [SMILES: N[C@@H](Cc1cccc2ccccc12)C(=O)O] |
| 2Ni | 3-(2-naphthyl)alanine | 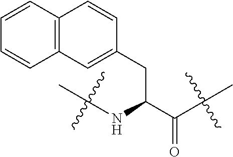 [SMILES: *N[C@@H](Cc1ccc2ccccc2c1)C(*)=O] | 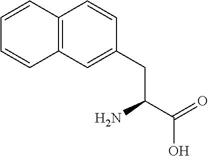 [SMILES: N[C@@H](Cc1ccc2ccccc2c1)C(=O)O] |
| 2Qi | 3-(2-quinolinyl)alanine | 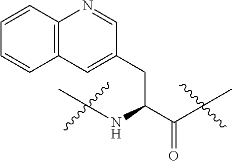 [SMILES: *N[C@@H](Cc1cnc2ccccc2c1)C(*)=O] | 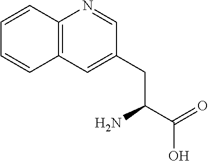 [SMILES: N[C@@H](Cc1cnc2ccccc2c1)C(=O)O] |
| 4Tfp | 4-trans-fluoro-proline | 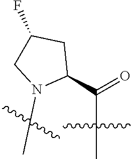 [SMILES: *C(=O)[C@@H]1C[C@@H](F)CN1*] | 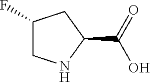 |
| 5Brw | 5-bromo-tryptophane | 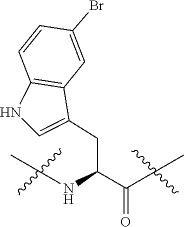 [SMILES: *N[C@@H](Cc1c[nH]c2ccc(Br)cc12)C(*)=O] | 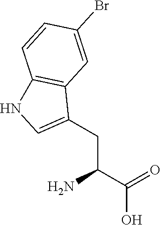 [SMILES: N[C@@H](Cc1c[nH]c2ccc(Br)cc12)C(=O)O] |
| 5Clw | 5-chloro-tryptophane | 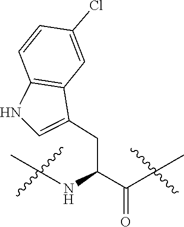 [SMILES: *N[C@@H](Cc1c[nH]c2ccc(Cl)cc12)C(*)=O] | 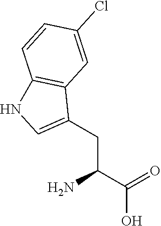 [SMILES: N[C@@H](Cc1c[nH]c2ccc(Cl)cc12)C(=O)O] |
| 6Clw | 6-chloro-tryptophane | 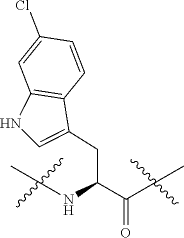 [SMILES: *N[C@@H](Cc1c[nH]c2cc(Cl)ccc12)C(*)=O] | 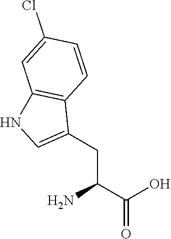 [SMILES: N[C@@H](Cc1c[nH]c2cc(Cl)ccc12)C(=O)O] |
| AF488 | Alexa Fluor 488 dye | 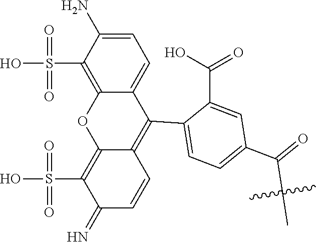 [SMILES: *C(=O)c1ccc(-c2c3ccc(=N)c(S(=O)(=O)O)c-3oc3c(S(=O)(=O)O)c(N)ccc23)c(C(=O)O)c1] | 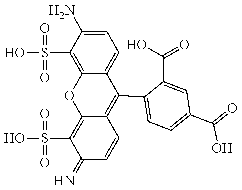 [SMILES: N=c1ccc2c(-c3ccc(C(=O)O)cc3C(=O)O)c3ccc(N)c(S(=O)(=O)O)c3oc-2c1S(=O)(=O)O] |
| AF488Ahx | AF488 bound to 6-amino hexanoic acid via dibenzocyclooctyne triazole | 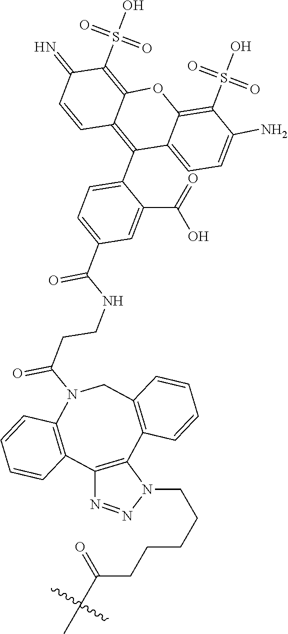 [SMILES: *C(=O)CCCCCn1nnc2c1-c1ccccc1CN(C(=O)CCNC(=O)c1ccc(-c3c4ccc(=N)c(S(=O)(=O)O)c-4oc4c(S(=O)(=O)O)c(N)ccc34)c(C(=O)O)c1)c1ccccc1-2] | 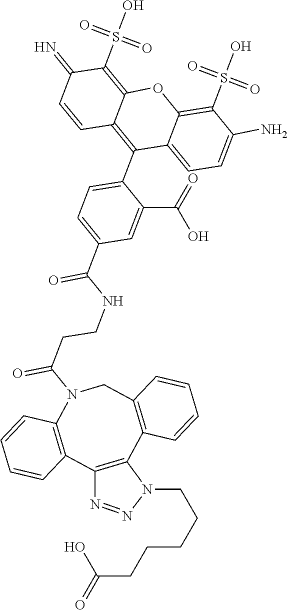 [SMILES: N=c1ccc2c(-c3ccc(C(=O)NCCC(=O)N4Cc5ccccc5-c5c(nnn5CCCCCC(=O)O)-c5ccccc54)cc3C(=O)O)c3ccc(N)c(S(=O)(=O)O)c3oc-2c1S(=O)(=O)O] |
| AF488N3K | AF488 bound to lysine hexanoic acid via dibenzocyclooctyne triazole | 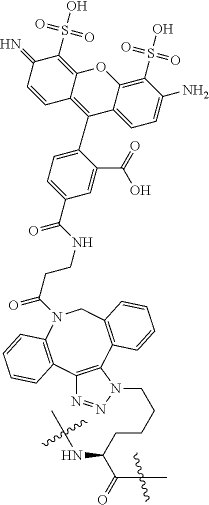 [SMILES: *N[C@@H](CCCCn1nnc2c1-c1ccccc1CN(C(=O)CCNC(=O)c1ccc(-c3c4ccc(=N)c(S(=O)(=O)O)c-4oc4c(S(=O)(=O)O)c(N)ccc34)c(C(=O)O)c1)c1ccccc1-2)C(*)=O] | 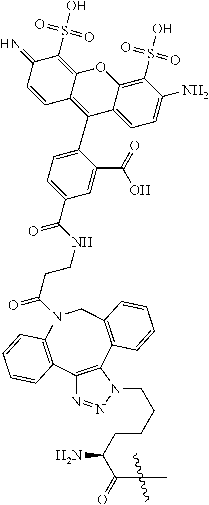 [SMILES: *C(=O)[C@@H](N)CCCCn1nnc2c1-c1ccccc1CN(C(=O)CCNC(=O)c1ccc(-c3c4ccc(=N)c(S(=O)(=O)O)c-4oc4c(S(=O)(=O)O)c(N)ccc34)c(C(=O)O)c1)c1ccccc1-2] |
| Ahx | 6-amino-hexanoic acid | 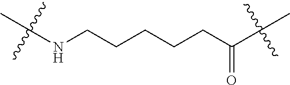 [SMILES: *NCCCCCC(*)=O] | 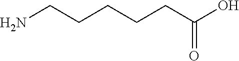 |
| Aib | 2-aminoisobutyric acid | 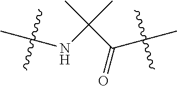 [SMILES: *NC(C)(C)C(*)=O] | 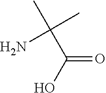 |
| ala | D-alanine | 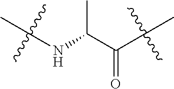 [SMILES: *N[C@H](C)C(*)=O] | 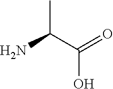 |
| Ala | alanine | 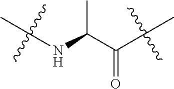 [SMILES: *N[C@@H](C)C(*)=O] | 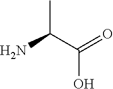 |
| amd | α-methyl-D-aspartic acid | 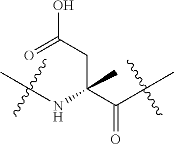 | 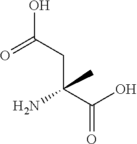 |
| Amd | α-methyl-aspartic acid | 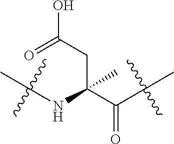 | 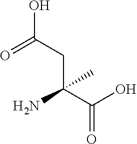 |
| Amf | α-methyl-phenyl alanine | 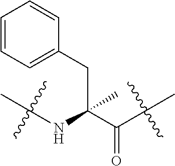 | 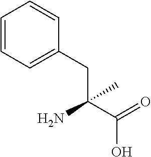 |
| ams | α-methyl-D-serine | 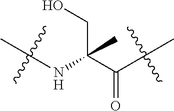 | 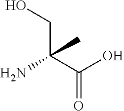 |
| Ams | α-methyl-serine | 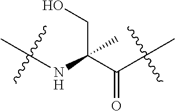 | 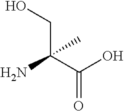 |
| APAc | (4-aminopiperidin-1- yl)acetic acid | 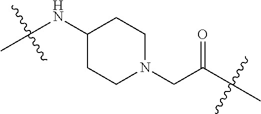 [SMILES: *NC1CCN(CC(*)=O)CC1] | 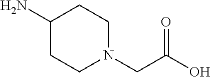 [SMILES: NC1CCN(CC(=O)O)CC1] |
| Apc | 4-amino-4- piperidinecarboxylic acid | 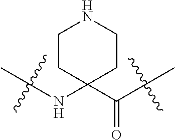 [SMILES: *NC1(C(*)=O)CCNCC1] | 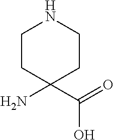 |
| Ape | 1,5-diaminopentane | 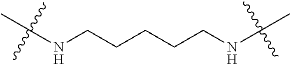 [SMILES: *NCCCCCN*] | 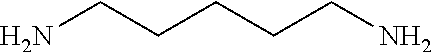 |
| Aph | 4-amino-phenylalanine | 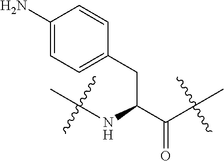 [SMILES: *N[C@@H](Cc1ccc(N)cc1)C(*)=O] | 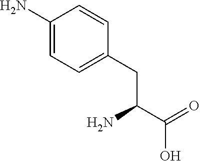 [SMILES: Nc1ccc(C[C@H](N)C(=O)O)cc1] |
| arg | D-arginine | 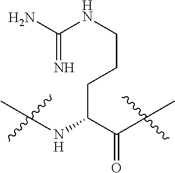 [SMILES: *N[C@H](CCCNC(=N)N)C(*)=O] | 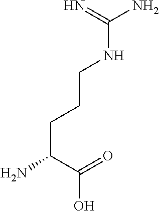 [SMILES: N=C(N)NCCC[C@@H](N)C(=O)O] |
| Arg | arginine | 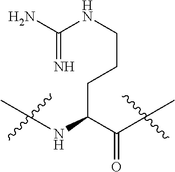 [SMILES: *N[C@@H](CCCNC(=N)N)C(*)=O] | 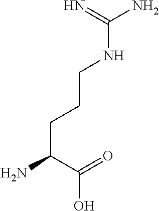 [SMILES: N=C(N)NCCC[C@H](N)C(=O)O] |
| Arg(Ac) | ω-acetyl-arginine | 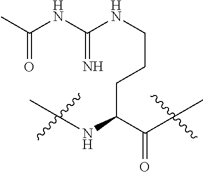 [SMILES: *N[C@@H](CCCNC(=N)NC(C)=O)C(*)=O] | 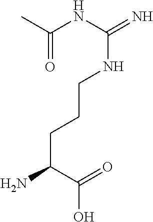 |
| Arg(EtCAyl) | | 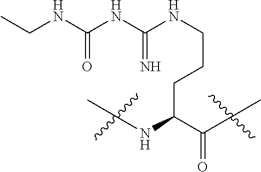 [SMILES: *N[C@@H](CCCNC(=N)NC(=O)NCC)C(*)=O] | 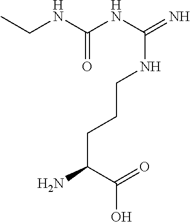 [SMILES: CCNC(=O)NC(=N)NCCC[C@H](N)C(=O)O] |
| Arg(Me) | ω-methyl-arginine | 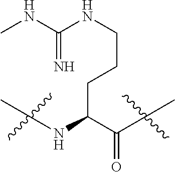 [SMILES: *N[C@@H](CCCNC(=N)NC)C(*)=O] | 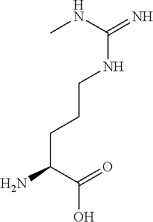 |
| asn | D-asparagine | 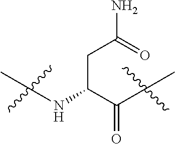 [SMILES: *N[C@H](CC(N)=O)C(*)=O] | 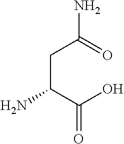 |
| Asn | asparagine | 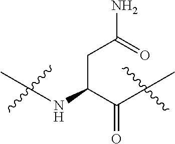 [SMILES: *N[C@@H](CC(N)=O)C(*)=O] | 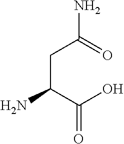 |
| asp | D-aspartic acid | 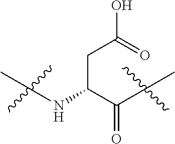 [SMILES: *N[C@H](CC(=O)O)C(*)=O] | 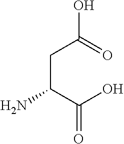 |
| Asp | aspartic acid | 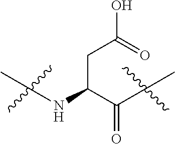 [SMILES: *N[C@@H](CC(=O)O)C(*)=O] | 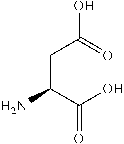 |
| Bal | ß-alanine | 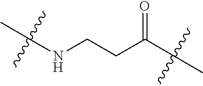 [SMILES: *NCCC(*)=O] | 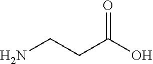 |
| Bio | D(+)-biotin | 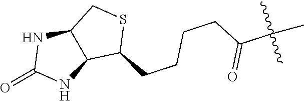 [SMILES: *C(=O)CCCC[C@@H]1SC[C@@H]2NC(=O)N[C@@H]21] | 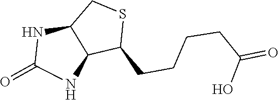 [SMILES: O=C(O)CCCC[C@@H]1SC[C@@H]2NC(=O)N[C@@H]21] |
| Bzl | benzyl | 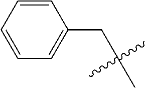 [SMILES: *Cc1ccccc1] | |
| Cit | citrulline | 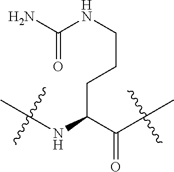 [SMILES: *N[C@@H](CCCNC(N)=O)C(*)=O] | 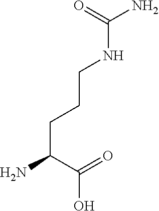 [SMILES: NC(=O)NCCC[C@H](N)C(=O)O] |
| Cmp | 4-carboxymethyl- piperidine | 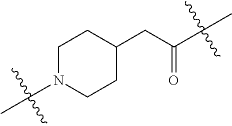 [SMILES: *C(=O)CC1CCN(*)CC1] | 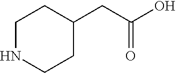 |
| Crown | Crown chelator | 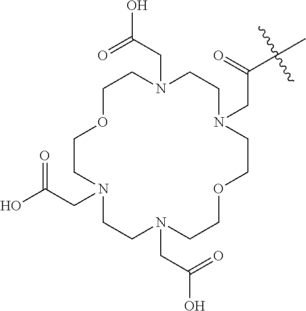 [SMILES: *C(=O)CN1CCOCCN(CC(=O)O)CCN(CC(=O)O)CCOCCN(CC(=O)O)CC1] | 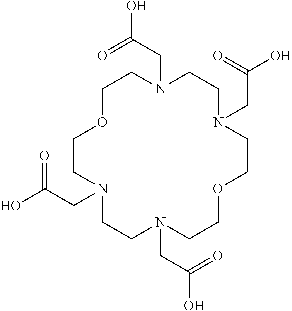 [SMILES: O=C(O)CN1CCOCCN(CC(=O)O)CCN(CC(=O)O)CCOCCN(CC(=O)O)CC1] |
| cys | D-cysteine | 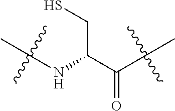 [SMILES: *N[C@H](CS)C(*)=O] | 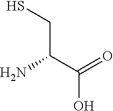 |
| Cys | cysteine | 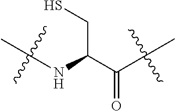 [SMILES: *N[C@@H](CS)C(*)=O] | 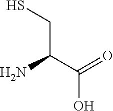 |
| DOTA | 2,2′,2″,2′′'-(1,4,7,10- tetraazacyclododecane- 1,4,7,10-tetrayl)tetraacetic acid | 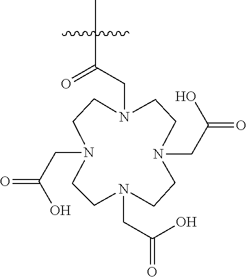 [SMILES: *C(=O)CN1CCN(CC(=O)O)CCN(CC(=O)O)CCN(CC(=O)O)CC1] | 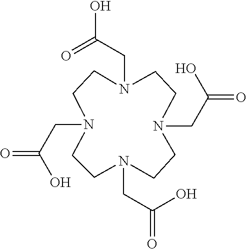 [SMILES: O=C(O)CN1CCN(CC(=O)O)CCN(CC(=O)O)CCN(CC(=O)O)CC1] |
| DOTAGA | 2-[1,4,7,10- tetraazacyclododecane- 4,7,10-tris(t-butyl- acetate)]-pentanedioic acid | 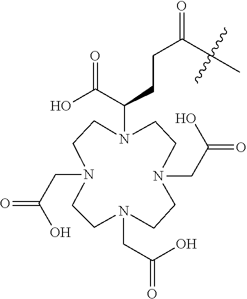 [SMILES: *C(=O)CC[C@H](C(=O)O)N1CCN(CC(=O)O)CCN(CC(=O)O)CCN(CC(=O)O)CC1] | 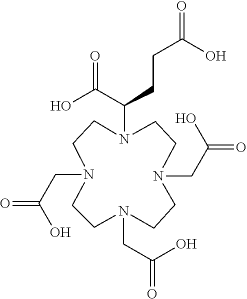 [SMILES: O=C(O)CC[C@H](C(=O)O)N1CCN(CC(=O)O)CCN(CC(=O)O)CCN(CC(=O)O)CC1] |
| DOTAM | 2-(4,7,10-tris(2-amino-2- oxoethyl)-1,4,7,10- tetraazacyclododecan-1- yl)acetic acid | 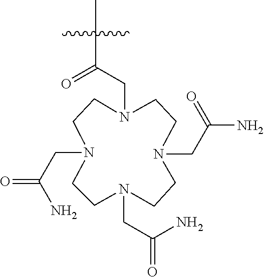 [SMILES: *C(=O)CN1CCN(CC(N)=O)CCN(CC(N)=O)CCN(CC(N)=O)CC1] | 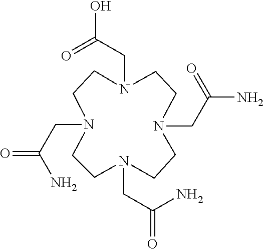 [SMILES: NC(=O)CN1CCN(CC(N)=O)CCN(CC(=O)O)CCN(CC(N)=O)CC1] |
| Deg | di-ethyl-glycine | 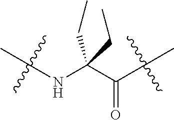 | 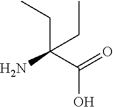 |
| Dfp | 4,4′-difluoroproline | 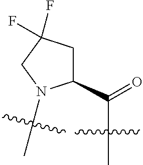 [SMILES: *C(=O)[C@@H]1CC(F)(F)CN1*] | 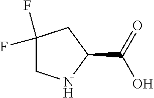 |
| Dtc | 5,5-dimethylthiazolidine- 4-carboxylic acid | 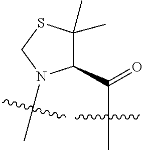 [SMILES: *C(=O)[C@H]1N(*)CSC1(C)C] | 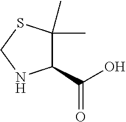 |
| Eaa | 3,4-dichloro- phenylalanine | 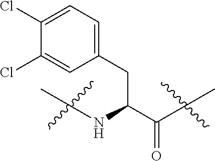 [SMILES: *N[C@@H](Cc1ccc(Cl)c(Cl)c1)C(*)=O] | 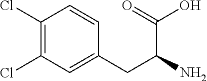 [SMILES: N[C@@H](Cc1ccc(Cl)c(Cl)c1)C(=O)O] |
| Egd | ω,ω'-dimethyl-arginine | 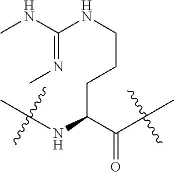 [SMILES: *N[C@@H](CCCN/C(=N/C)NC)C(*)=O] | 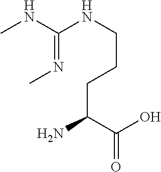 |
| Eew | ω-nitro-arginine | 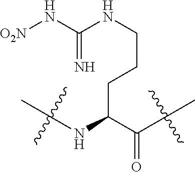 [SMILES: *N[C@@H](CCCNC(=N)N[N+](=O)[O-])C(*)=O] | 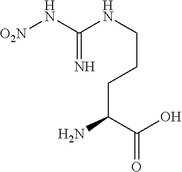 [SMILES: N=C(NCCC[C@H](N)C(=O)O)N[N+](=O)[O-]] |
| en | ethylene diamine as linker | 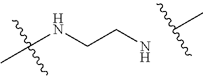 | 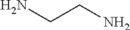 |
| en | ethylene diamine as C-terminal building block | 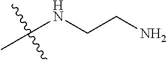 [SMILES: *NCCN] | 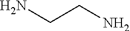 |
| en(Me) | N-methylethylene diamine | 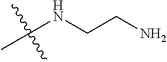 [SMILES: *NCCN] | 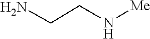 |
| en(Me)₂ | unsym-dimethylethylene diamine | 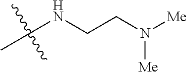 [SMILES: *NCCN(C)C] | 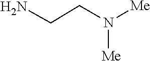 |
| EtCAyl | ethyl carbamoyl | 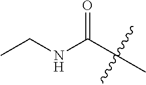 [SMILES: *C(=O)NCC] | |
| EuDOTA | Eu(III) DOTA complex | 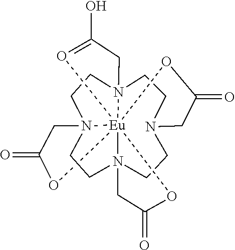 | |
| Fac | 4-carboxymethyl- phenylalanine | 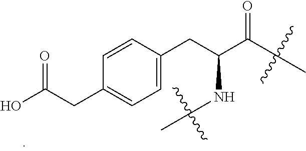 [SMILES: *N[C@@H](Cc1ccc(CC(=O)O)cc1)C(*)=O] | 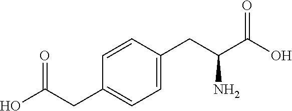 [SMILES: N[C@@H](Cc1ccc(CC(=O)O)cc1)C(=O)O] |
| Fso | 4-sulfo-phenylalanine | 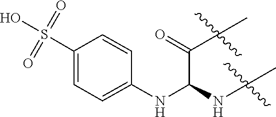 [SMILES: *N[C@@H](Nc1ccc(S(=O)(=O)O)cc1)C(*)=O] | 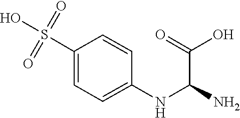 [SMILES: N[C@@H](Nc1ccc(S(=O)(=O)O)cc1)C(=O)O] |
| Gab | γ-aminobutyric acid | 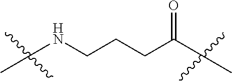 [SMILES: *NCCCC(*)=O] | 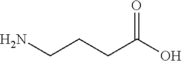 |
| GaDOTA | Ga(III) DOTA complex | 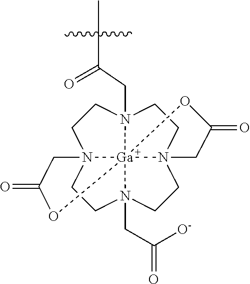 | |
| Gln | glutamine | 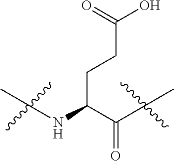 [SMILES: *N[C@@H](CCC(=O)O)C(*)=O] | 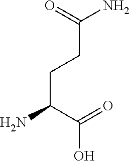 |
| Gln(Gu) | | 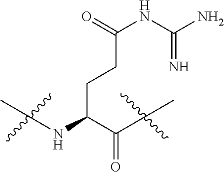 [SMILES: *N[C@@H](CCC(=O)NC(=N)N)C(*)=O] | 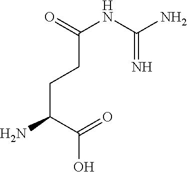 [SMILES: N=C(N)NC(=O)CC[C@H](N)C(=O)O] |
| glu | D-glutamic acid | 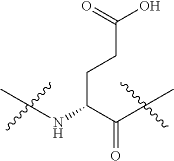 [SMILES: *N[C@H](CCC(=O)O)C(*)=O] | 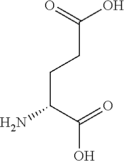 |
| Glu | glutamic acid | 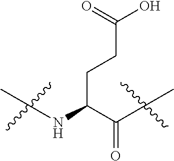 [SMILES: *N[C@@H](CCC(=O)O)C(*)=O] | 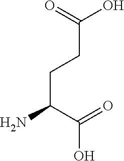 |
| Gly | gylcine | 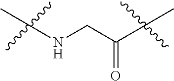 [SMILES: *NCC(*)=O] | 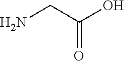 |
| Gu | guanidinyl | 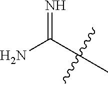 [SMILES: *C(=N)N] | |
| HPA | 3-hydroxy-propionic acid | 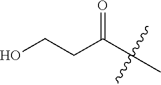 [SMILES: *C(=O)CCO] | 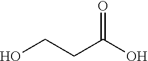 |
| Har | homoarginine | 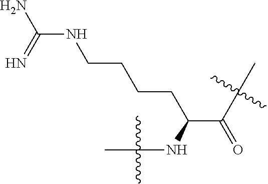 [SMILES: *N[C@@H](CCCCNC(=N)N)C(*)=O] | 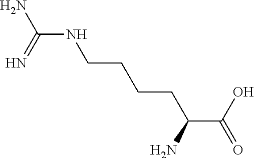 [SMILES: N=C(N)NCCCC[C@H](N)C(=O)O] |
| Hex | hexanoic acid | 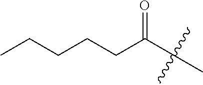 [SMILES: *C(=O)CCCCC] | 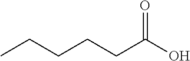 |
| Hfe | homo-phenylalanine | 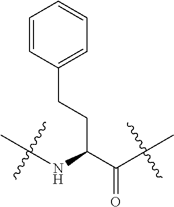 [SMILES: *N[C@@H](CCc1ccccc1)C(*)=O] | 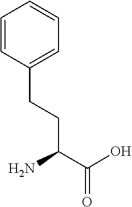 [SMILES: N[C@@H](CCc1ccccc1)C(=O)O] |
| Hgn | homo-glutamine | 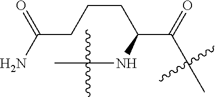 [SMILES: *N[C@@H](CCCC(N)=O)C(*)=O] | 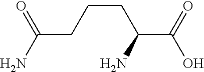 [SMILES: NC(=O)CCC[C@H](N)C(=O)O] |
| Hib | 2-hydroxy isobutyric acid | 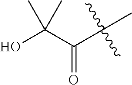 [SMILES: *C(=O)C(C)(C)O] | 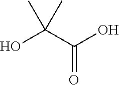 |
| Hse | homo serine | 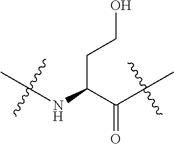 [SMILES: *N[C@@H](CCO)C(*)=O] | 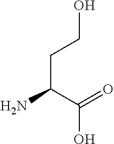 |
| HYDAc | hydantoin-5-acetic acid | 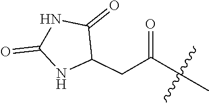 [SMILES: *C(=O)CC1NC(=O)NC1=O] | 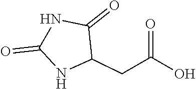 [SMILES: O=C(O)CC1NC(=O)NC1=O] |
| Hyp | 4-hydroxyproline | 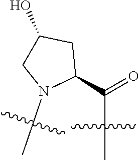 [SMILES: *C(=O)[C@@H]1C[C@@H](O)CN1*] | 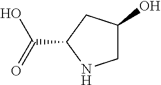 |
| Ile | isoleucine | 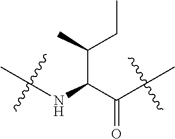 [SMILES: *N[C@H](C(*)=O)[C@@H](C)CC] | 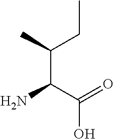 |
| InDOTA | In(III) DOTA complex | 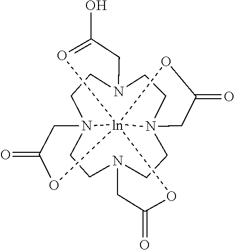 | |
| Iva | isovaleric acid | 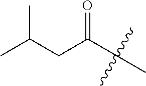 [SMILES: *C(=O)CC(C)C] | 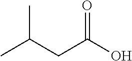 |
| Kip | ϵ-iso-propyl-lysine | 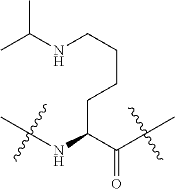 [SMILES: *N[C@@H](CCCCNC(C)C)C(*)=O] | 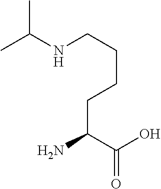 |
| KMe2 | ϵ-di-methyl-lysine | 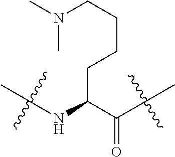 [SMILES: *N[C@@H](CCCCN(C)C)C(*)=O] | 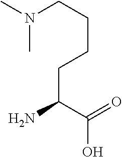 |
| KMe3 | ϵ-tri-methyl-lysine | 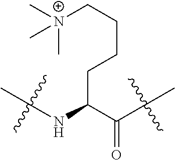 [SMILES: *N[C@@H](CCCC[N+](C)(C)C)C(*)=O] | 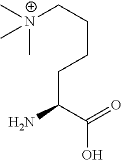 |
| leu | D-leucine | 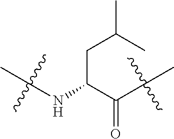 [SMILES: *N[C@H](CC(C)C)C(*)=O] | 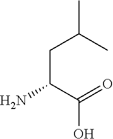 |
| Leu | leucine | 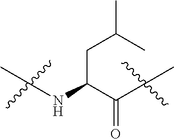 [SMILES: *N[C@@H](CC(C)C)C(*)=O] | 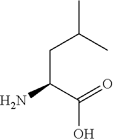 |
| LuDOTA | Lu(III) DOTA complex | 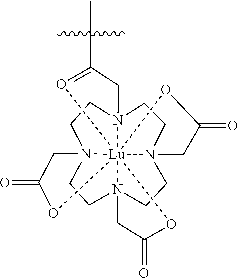 | |
| LSC | 2,2′,2″-(10-(2-amino-2- oxoethyl)-1,4,7,10- tetraazacyclododecane- 1,4,7-triyl)triacetic acid | 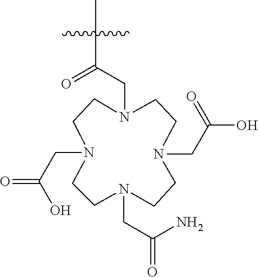 [SMILES: *C(=O)CN1CCN(CC(=O)O)CCN(CC(N)=O)CCN(CC(=O)O)CC1] | 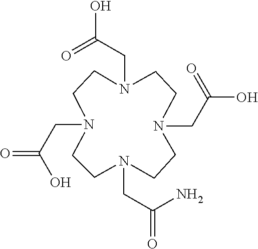 [SMILES: NC(=O)CN1CCN(CC(=O)O)CCN(CC(=O)O)CCN(CC(=O)O)CC1] |
| lys | D-lysine | 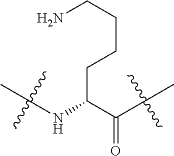 [SMILES: *N[C@H](CCCCN)C(*)=O] | 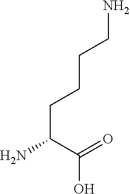 |
| Lys | lysine | 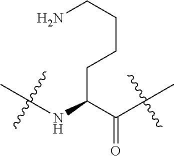 [SMILES: *N[C@@H](CCCCN)C(*)=O] | 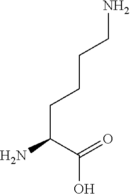 |
| Lys(Me) | ϵ-methyl-lysine | 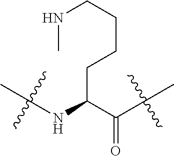 [SMILES: *N[C@@H](CCCCNC)C(*)=O] | 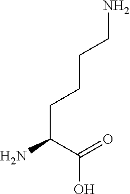 |
| Macropa | Macropa chelator with additional nitrogen derivatized with glutaric acid as linker | 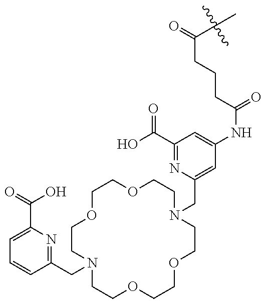 [SMILES: *C(=O)CCCC(=O)Nc1cc(CN2CCOCCOCCN(Cc3cccc(C(=O)O)n3)CCOCCOCC2)nc(C(=O)O)c1] | 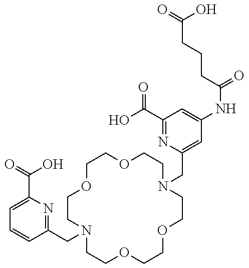 [SMILES: O=C(O)CCCC(=O)Nc1cc(CN2CCOCCOCCN(Cc3cccc(C(=O)O)n3)CCOCCOCC2)nc(C(=O)O)c1] |
| Mcf | meta-chloro-phenylalanine | 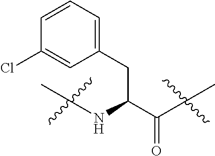 [SMILES: *N[C@@H](Cc1cccc(Cl)c1)C(*)=O] | 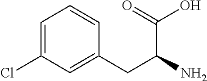 [SMILES: N[C@@H](Cc1cccc(Cl)c1)C(=O)O] |
| Me | methyl | 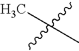 | |
| Met | methionine | 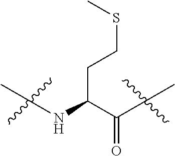 [SMILES: *N[C@@H](CCSC)C(*)=O] | 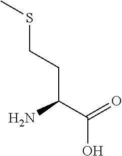 |
| Miy | meta-iodo-tyrosine | 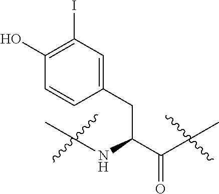 [SMILES: *N[C@@H](Cc1ccc(O)c(I)c1)C(*)=O] | 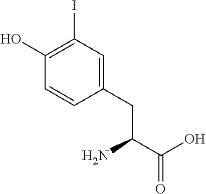 [SMILES: N[C@@H](Cc1ccc(O)c(I)c1)C(=O)O] |
| Mff | 3-fluoro-phenylalanine | 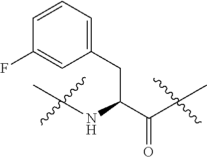 [SMILES: *N[C@@H](Cc1cccc(F)c1)C(*)=O] | 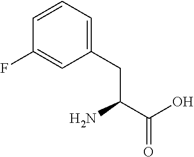 [SMILES: N[C@@H](Cc1cccc(F)c1)C(=O)O] |
| Mmf | 3-methyl-phenylalanine | 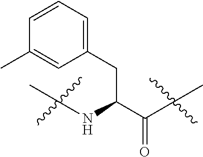 [SMILES: *N[C@@H](Cc1cccc(C)c1)C(*)=O] | 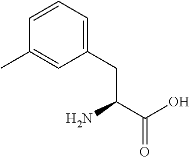 |
| Mnf | 3-cyano-phenylalanine | 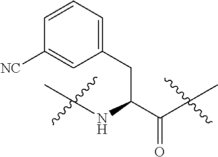 [SMILES: *N[C@@H](Cc1cccc(C#N)c1)C(*)=O] | 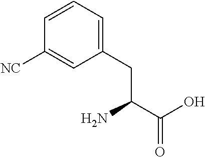 [SMILES: N#Cc1cccc(C[C@H](N)C(=O)O)c1] |
| Mtf | 3-trifluoromethyl- phenylalanine | 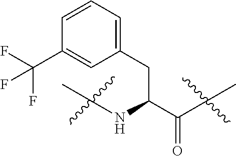 [SMILES: *N[C@@H](Cc1cccc(C(F)(F)F)c1)C(*)=O] | 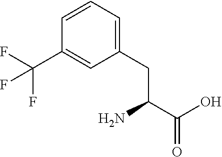 [SMILES: N[C@@H](Cc1cccc(C(F)(F)F)c1)C(=O)O] |
| Mpa | 3-pyridyl-alanine | 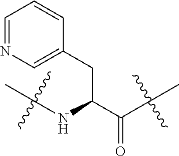 [SMILES: *N[C@@H](Cc1cccnc1)C(*)=O] | 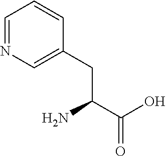 [SMILES: N[C@@H](Cc1cccnc1)C(=O)O] |
| N3Ahx | 6-azido-hexanoic acid | 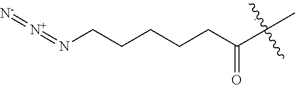 [SMILES: *C(=O)CCCCCN=[N+]=[N-]] | 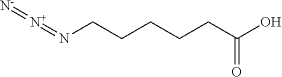 [SMILES: [N-]=[N+]=NCCCCCC(=O)O] |
| NHBu | n-butylamine at C-terminus | 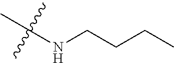 [SMILES: *NCCCC] | 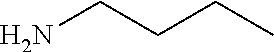 |
| NHnPen | neo-pentylamine at C-terminus | 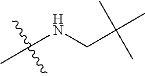 [SMILES: *NCC(C)(C)C] | 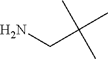 |
| Nle | norleucine | 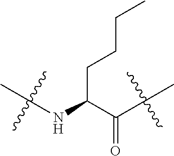 [SMILES: *N[C@@H](CCCC)C(*)=O] | 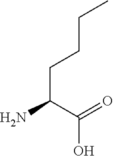 |
| Nmf | N-methyl-phenylalanine | 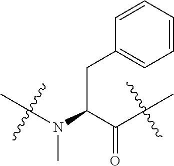 [SMILES: *C(=O)[C@H](Cc1ccccc1)N(*)C] | 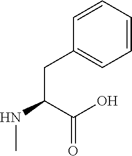 |
| Nmg | N-methyl-glycine | 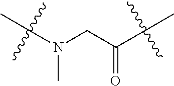 [SMILES: *C(=O)CN(*)C] | 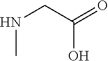 |
| Nmk | N-α-methyl-lysine | 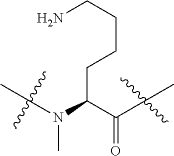 [SMILES: *C(=O)[C@H](CCCCN)N(*)C] | 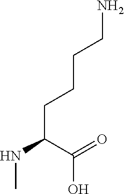 |
| Nml | N-methyl-leucine | 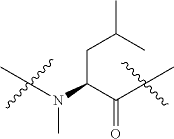 [SMILES: *C(=O)[C@H](CC(C)C)N(*)C] | 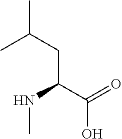 |
| Nmn | N-methyl-asparagine | 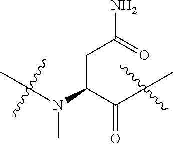 [SMILES: *C(=O)[C@H](CC(N)=O)N(*)C] | 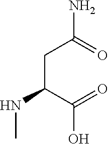 |
| Nmr | N-methyl-arginine | 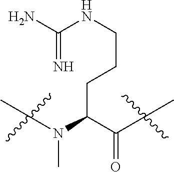 [SMILES: *C(=O)[C@H](CCCNC(=N)N)N(*)C] | 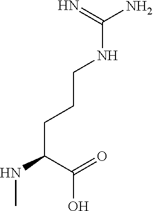 |
| Nmt | N-methyl-threonine | 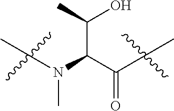 [SMILES: *C(=O)[C@H]([C@@H](C)O)N(*)C] | 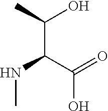 |
| Npg | neo-pentylglycine | 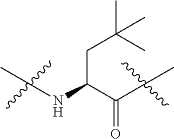 [SMILES: *N[C@@H](CC(C)(C)C)C(*)=O] | 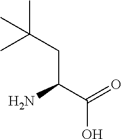 |
| O2Oc | [2-(2- amino- ethoxy)ethoxy]acetic acid | 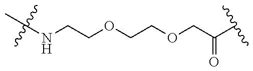 [SMILES: *NCCOCCOCC(*)=O] | 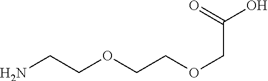 [SMILES: NCCOCCOCC(=O)O] |
| Ocf | ortho-chlorophenylalanine | 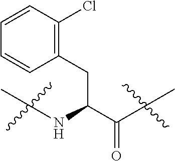 [SMILES: *N[C@@H](Cc1ccccc1Cl)C(*)=O] | 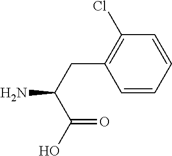 [SMILES: N[C@@H](Cc1ccccc1Cl)C(=O)O] |
| Oic | octahydro-1H-indole-2- carboxylic acid | 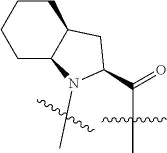 [SMILES: *C(=O)[C@@H]1C[C@@H]2CCCC[C@@H]2N1*] | 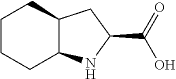 [SMILES: O=C(O)[C@@H]1C[C@@H]2CCCC[C@@H]2N1] |
| Opa | 2-pyridyl-alanine | 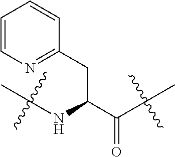 [SMILES: *N[C@@H](Cc1ccccn1)C(*)=O] | 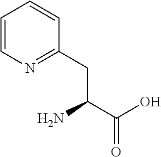 [SMILES: N[C@@H](Cc1ccccn1)C(=O)O] |
| Opy | γ-2-pyridyl-ornithine | 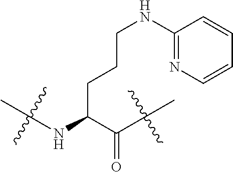 [SMILES: *N[C@@H](CCCNc1ccccn1)C(*)=O] | 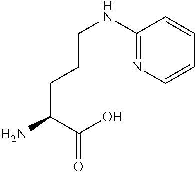 [SMILES: N[C@@H](CCCNc1ccccn1)C(=O)O] |
| Orn | ornithine | 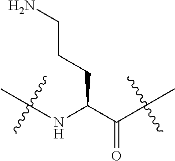 [SMILES: *N[C@@H](CCCN)C(*)=O] | 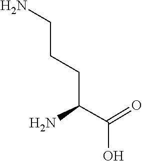 |
| Pab | 4-amino-benzoic acid | 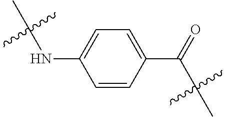 [SMILES: *Nc1ccc(C(*)=O)cc1] | 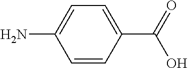 |
| Pam | α-methyl-proline | 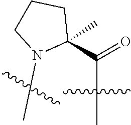 [SMILES: *C(=O)[C@]1(C)CCCN1*] | 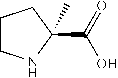 |
| Pamb | 4-aminomethyl-benzoic acid | 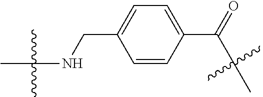 [SMILES: *NCc1ccc(C(*)=O)cc1] | 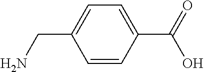 [SMILES: NCc1ccc(C(=O)O)cc1] |
| Pamp | 6-aminomethyl-pyridine- 3-carboxylic acid | 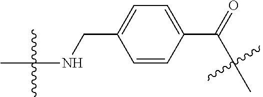 [SMILES: *NCc1ccc(C(*)=O)cc1] | 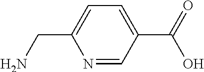 [SMILES: NCc1ccc(C(=O)O)cn1] |
| Pcf | para-chloro-phenylalanine | 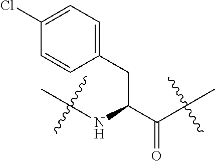 [SMILES: *N[C@@H](Cc1ccc(Cl)cc1)C(*)=O] | 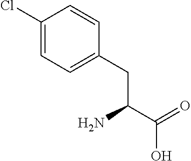 [SMILES: N[C@@H](Cc1ccc(Cl)cc1)C(=O)O] |
| Pff | 4-fluoro-phenylalanine | 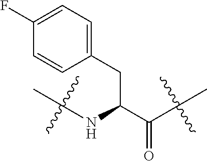 [SMILES: *N[C@@H](Cc1ccc(F)cc1)C(*)=O] | 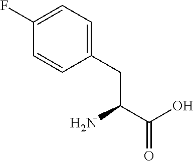 [SMILES: N[C@@H](Cc1ccc(F)cc1)C(=O)O] |
| phe | D-phenylalanine | 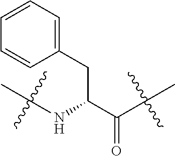 [SMILES: *N[C@H](Cc1ccccc1)C(*)=O] | 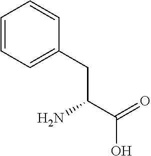 [SMILES: N[C@H](Cc1ccccc1)C(=O)O] |
| Phe | phenylalanine | 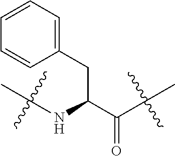 [SMILES: *N[C@@H](Cc1ccccc1)C(*)=O] | 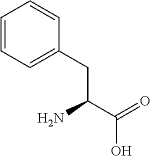 [SMILES: N[C@@H](Cc1ccccc1)C(=O)O] |
| Pmf | 4-methyl-phenylalanine | 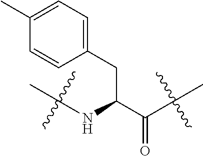 [SMILES: *N[C@@H](Cc1ccc(C)cc1)C(*)=O] | 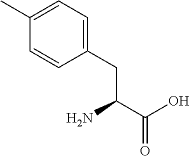 |
| Pnf | 4-cyano-phenylalanine | 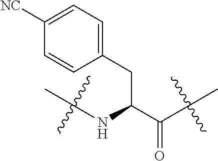 [SMILES: *N[C@@H](Cc1ccc(C#N)cc1)C(*)=O] | 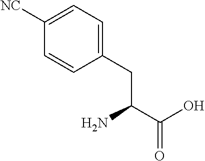 [SMILES: N#Cc1ccc(C[C@H](N)C(=O)O)cc1] |
| Ppa | 4-pyridyl-alanine | 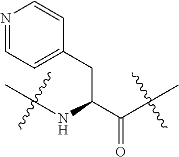 [SMILES: *N[C@@H](Cc1ccncc1)C(*)=O] | 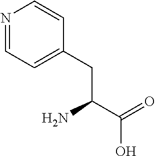 [SMILES: N[C@@H](Cc1ccncc1)C(=O)O] |
| PPAc | 4-carboxymethyl piperazine | 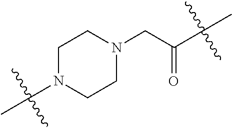 [SMILES: *C(=O)CN1CCN(*)CC1] | 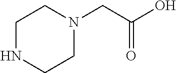 |
| Pro | proline | 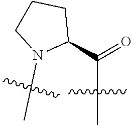 [SMILES: *C(=O)[C@@H]1CCCN1*] | 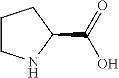 |
| Ptf | 4-trifluoromethyl- phenylalanine | 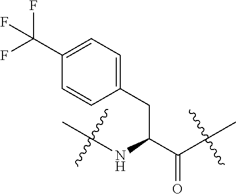 [SMILES: *N[C@@H](Cc1ccc(C(F)(F)F)cc1)C(*)=O] | 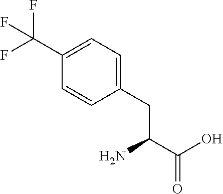 [SMILES: N[C@@H](Cc1ccc(C(F)(F)F)cc1)C(=O)O] |
| RMe1 | δ-methyl-arginine | 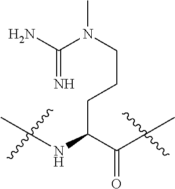 [SMILES: *N[C@@H](CCCN(C)C(=N)N)C(*)=O] | 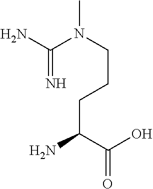 |
| RMe2 | ω,ω-dimethyl-arginine | 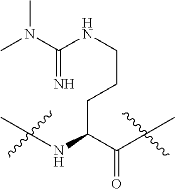 [SMILES: *N[C@@H](CCCNC(=N)N(C)C)C(*)=O] | 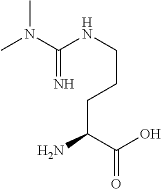 |
| RMe2a | δ,ω-dimethyl-arginine | 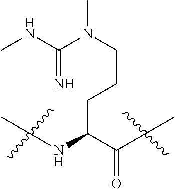 [SMILES: *N[C@@H](CCCN(C)C(=N)NC)C(*)=O] | 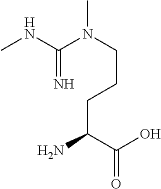 |
| RMe3 | δ,ω,ω′-trimethyl-arginine | 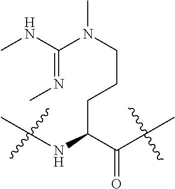 [SMILES: *N[C@@H](CCCN(C)/C(=N/C)NC)C(*)=O] | 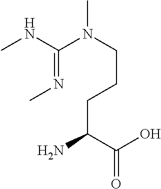 |
| SaPr | 3-sulfamoylpropanoic acid | 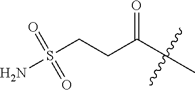 [SMILES: *C(=O)CCS(N)(=O)=O] | 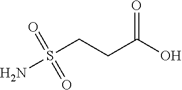 |
| Ser | serine | 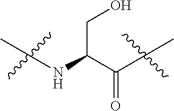 [SMILES: *N[C@@H](CO)C(*)=O] | 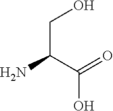 |
| Smc | S-methyl-cysteine | 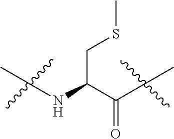 [SMILES: *N[C@@H](CSC)C(*)=O] | 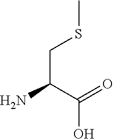 |
| Succinyl | succinic acid (as linker) | 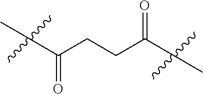 [SMILES: *C(=O)CCC(*)=O] | 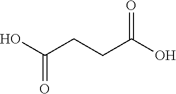 |
| Tap | 4-trans-aminoproline | 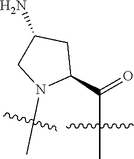 [SMILES: *C(=O)[C@@H]1C[C@@H](N)CN1*] | 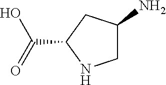 |
| thr | D-theronine | 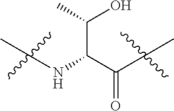 [SMILES: *N[C@@H](C(*)=O)[C@H](C)O] | 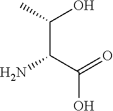 |
| Thr | threonine | 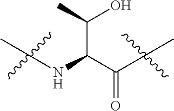 [SMILES: *N[C@H](C(*)=O)[C@@H](C)O] | 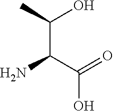 |
| Throl | threoninol | 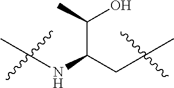 [SMILES: *C[C@@H](N*)[C@@H](C)O] | 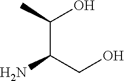 |
| Throl-OH | threoninol | 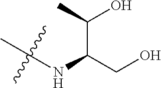 [SMILES: *N[C@H](CO)[C@@H](C)O] | 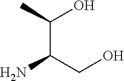 |
| Tle | tert-leucine | 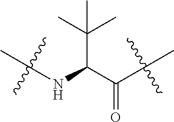 [SMILES: *N[C@H](C(*)=O)C(C)(C)C] | 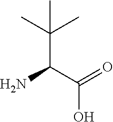 |
| Trp | tryptophane | 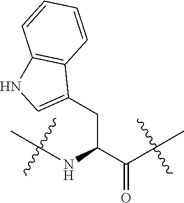 [SMILES: *N[C@@H](Cc1c[nH]c2ccccc12)C(*)=O] | 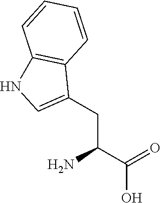 [SMILES: N[C@@H](Cc1c[nH]c2ccccc12)C(=O)O] |
| Ttds | 4,7,10-trioxa-14- azaoctadecan-18-oic acid | 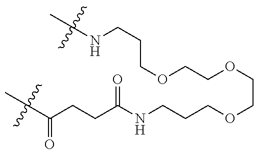 [SMILES: *NCCCOCCOCCOCCCNC(=O)CCC(*)=O] | 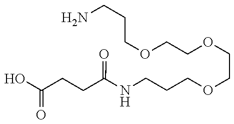 [SMILES: NCCCOCCOCCOCCCNC(=O)CCC(=O)O] |
| Tyr | tyrosine | 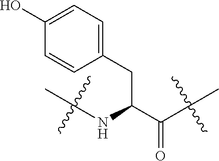 [SMILES: *N[C@@H](Cc1ccc(O)cc1)C(*)=O] | 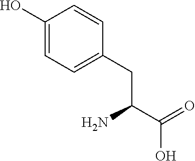 [SMILES: N[C@@H](Cc1ccc(O)cc1)C(=O)O] |
| Urr | 3-keto-4-aza-arginine | 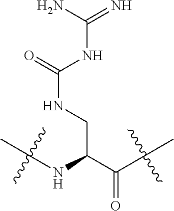 [SMILES: *N[C@@H](CNC(=O)NC(=N)N)C(*)=O] | 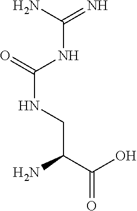 [SMILES: N=C(N)NC(=O)NC[C@H](N)C(=O)O] |
| Val | valine | 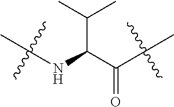 [SMILES: *N[C@H](C(*)=O)C(C)C] | 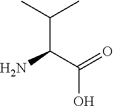 |

The amino acid sequences of the peptides provided herein are depicted in typical peptide sequence format, as would be understood by the ordinary skilled artisan. For example, the three-letter code of a conventional amino acid, or the code for a non-conventional amino acid or the abbreviations for additional building blocks, indicates the presence of the amino acid or building block in a specified position within the peptide sequence. The code for each amino acid or building block is connected to the code for the next and/or previous amino acid or building block in the sequence by a hyphen which (typically represents an amide linkage).

Where an amino acid contains more than one amino and/or carboxy group all orientations of this amino acid are in principle possible, but in α-amino acid the utilization of the α-amino and the α-carboxy group is preferred and otherwise preferred orientations are explicitly specified.

For amino acids, in their abbreviations the first letter indicates the stereochemistry of the C-α-atom if applicable. For example, a capital first letter indicates that the L-form of the amino acid is present in the peptide sequence, while a lower case first letter indicating that the D-form of the correspondent amino acid is present in the peptide sequence.

In an embodiment, and as preferably used herein, an aromatic amino acid is any kind of amino acid which comprises an aryl or heteroaryl group.

In an embodiment, and as preferably used herein, an aromatic α-amino acid is any kind of α-amino acid which comprises an aryl or heteroaryl group.

In an embodiment, and as preferably used herein, an α-amino acid is an amino acid wherein the amino and the carboxyl group are substituents of the same carbon atom.

Those skilled in the art will recognize if a stereocenter exists in the compounds disclosed herein irrespective thereof whether such stereocenter is part of an amino acid moiety or any other part or moiety of the compound of the disclosure. Accordingly, the present disclosure includes possible stereoisomers and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

In the present disclosure, the structural formula of the compound represents a certain isomer for convenience in some cases, but the present disclosure includes all isomers, such as geometrical isomers, optical isomers based on an asymmetrical carbon, stereoisomers, tautomers, and the like.

Unless indicated to the contrary, the amino acid sequences are presented herein in N- to C-terminus direction.

### Linear Peptides

A general linear peptide is typically written from the N- to C-terminal direction as shown below:

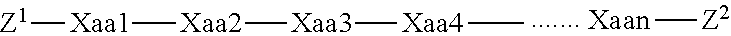

### Therein

1. Xaax is the abbreviation, descriptor or symbol for amino acids or building blocks at specific sequence position x as shown in Table 4, whereby the linear connection of individual Xaax is indicated by a hyphen,
2. Z¹ is a N-terminal group, which may be a chelator or an N-terminal group NT, e.g. ‘H’ (Hydrogen for a free N-terminal amino group) or an abbreviation for a specific terminating carboxylic acid like ‘Ac’ for acetic acid or other chemical group or structural formula of chemical groups linked to the N-terminal amino acid code (Xaa1) via a hyphen and
3. Z² is a C-terminal group which is typically is a C-terminal group CT such as ‘OH’ or ‘NH₂’ (as terminal carboxylic acid or amide), but which may also be an amino acid to which a chelator is attached via an optional linker.
   Branched Peptides with Side Chains Modified by Specific Building Blocks or Peptides

A general linear, branched peptide is written from the N- to C-terminal direction as shown below:

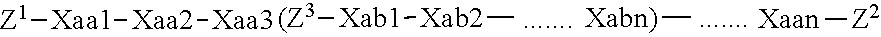

Therein the statements 1.-3. of the description of linear peptides for the specification of Xaax, Z¹ and Z² in the main chain of the branched peptide apply.

The position of a branch is specified by parentheses right next to a Xaax abbreviation. Branches typically occur at lysine (Lys) residues (or similar), which means that the branch is attached to side chain ε-amino function of the lysine via an amide bond.

The content of the parenthesis describes the sequence/structure of the peptide branch ‘Z³-Xab1-Xab2- . . . Xabn’. Herein
1. Xabx is the abbreviation, descriptor or symbol for amino acids or building blocks at specific sequence position x of said branch as shown in Table 4, whereby the linear connection of individual Xabx is indicated by a hyphen,
2. Z³ is a N-terminal group of said branch, which typically is a chelator linked to the N-terminal amino acid Xab1 or ‘H’, which indicates a free amino group of said Xab1, and
3. the last building block of said branch Xabn, which connects the branch with the main chain by forming an amide bond with its own carboxyl function with the side chain amino function of this lysine (or similar residue).

Furthermore the content of the parenthesis may be a chemical group (e.g. Me for a methyl group) attached to a specific hetero atom of the side chain of said building block/amino acid Xaax. For clarification such residues (e.g. Arg(Me) or Gln(Gu)) are comprised in Table 4.

### Cyclic Peptides

An exemplaric general cyclic peptide written from the N- to C-terminal direction is shown below:

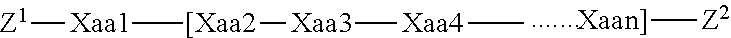

Therein the statements 1.-3. of the description of linear peptides for the specification of Xaax, Z¹ and Z² in the main chain of the cyclic peptide apply. The characteristics of the peptide cycle are specified by square brackets. Therein
1. the opening square bracket indicates the building block at whose side chain the cycle is initiated, and
2. the closing square bracket indicates the building block at whose side chain the cycle is terminated.

The chemical nature of the connection between these two resides is
1. typically a disulphide bond in case both of the residues/amino acids are cysteine and hence contain a sulfhydryl moiety, or
2. a thio-acetal connection in case one of said residues is an S-methyl cysteine and the other one is a cysteine.

Furthermore the cyclic peptide may contain branches at certain positions within its linear sequence. In the case the statements of ‘Branched peptides with side chains modified by specific building blocks or peptides’ apply.

As a non-limiting example, the structure of DOTA-Cmp-Thr-Aib-Phe-[Cys-Lys-Arg-Aib-Asn-Cys]-Tle-Thr-NH₂ (PSM-0492) is depicted below.

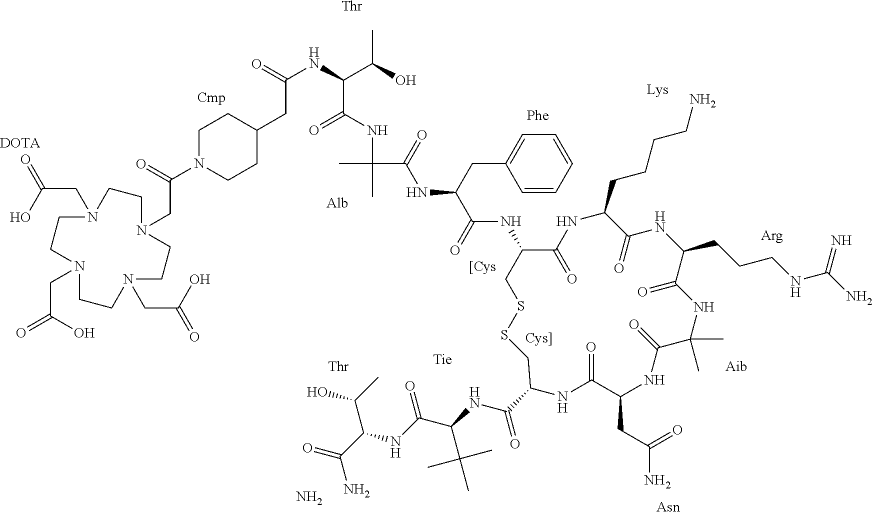

### Therein

1. The DOTA chelator corresponds to Z¹ in general Formula (I).
2. Cmp and Thr together form L¹ in general Formula (I).
3. Aib and Phe correspond to Xaa2 and Xaa3 respectively in general Formula (I).
4. Lys, Arg, Aib, and Asn correspond to Xaa5, Xaa6, Xaa7, and Xaa8 respectively in general Formula (I).
5. Tle corresponds to Xaa10 in general Formula (I).
6. Thr corresponds to L² in general Formula (I).
7. NH₂ corresponds to Z² in general Formula.
8. The opening square bracket (‘[’) left adjacent to the N-terminal cysteine in the sequence indicates that at this residue the cycle is initiated.
9. The closing square bracket (‘]’) adjacent to the N-terminal cysteine in the sequence indicates that at this residue the cycle is terminated.

In an embodiment, the compound of the invention comprises a chelator. Preferably, the chelator is part of the compound of the invention, whereby the chelator is either directly or indirectly such as by a linker attached to the compound of the invention. A preferred chelator is a chelator which forms metal chelates preferably comprising at least one radioactive metal. The at least one radioactive metal is preferably useful in or suitable for diagnostic and/or therapeutic and/or theranostic use and is more preferably useful in or suitable for imaging and/or radiotherapy.

Chelators in principle useful in and/or suitable for the practicing of the instant invention including diagnosis and/or therapy of a disease are known to the person skilled in the art. A wide variety of respective chelators is available and has been reviewed, e.g. by Banerjee et al. (Banerjee, et al., Dalton Trans, 2005, 24: 3886), and references therein (Price, et al., Chem Soc Rev, 2014, 43: 260; Wadas, et al., Chem Rev, 2010, 110: 2858). Such chelators include, but are not limited to linear, cyclic, macrocyclic, tetrapyridine, N3S, N2S2 and N4 chelators as disclosed in U.S. Pat. Nos. 5,367,080 A, 5,364,613 A, 5,021,556 A, 5,075,099 A and 5,886,142 A.

Representative chelators and their derivatives including any bifunctional versions that can be conjugated to the targeting vector include, but are not limited to the examples listed in Table 7.

TABLE 7
Examples of chelators with their corresponding chemical
names and exemplary reference of their disclosure.

| Chelator | Chemical name | Ref. |
|---|---|---|
| AAZTA | 1,4-bis(carboxymethyl)-6-[bis(carboxymethyl)]amino-6- | a |
| | methylperhydro-1,4-diazepine | |
| ATMP | (nitrilotris(methylene))tris(phosphonic acid) | aa |
| ATSM | Diacetyl bis(N⁴-methylthiosemicarbazone) | b |
| Aza crown ether | 1,10-dioxa-4,7,13,16-tetraazacyclooctadecane | dd |
| AZEP-DTPA | cis-(2,7-bis-(bis-carboxymethyl-amino)-methyl)-azepan-1-yl)- | c |
| | acetic | |
| BATA | 2,3,4,5,6,7,8,9,10,11,12,13,14,15-tetradecahydro- | c |
| | 1,16,4,7,10,13-benzodioxatetraaza-cyclooctadecine-4,7,10,13- | |
| | yl-tetraacetic acid | |
| BATPA | 1,2-bis(2-aminophenoxy)ethane-N,N,N′,N′-tetraacetic acid | s |
| BPCA | 2,2′,2″,2′″-(([2,2′-bipyridine]-6,6′- | a |
| | diylbis(methylene))bis(azanetriyl))tetraacetic acid | |
| Bp44mT | 2-benzoylpyridine 4,4-dimethyl-3-thiosemicarbazone | rr |
| C3B-DO2A | 2,2′-(1,4,7,10-tetraazabicyclo[5.5.3]pentadecane-4,10- | j |
| | diyl)diacetic acid | |
| CB-DO2A | 4,10-bis(carboxymethyl)-1,4,7,10- | a |
| | tetraazabicyclo[5.5.2]tetradecane | |
| CB-TE1A1P | 2-(11-(phosphonomethyl)-1,4,8,11- | a |
| | tetraazabicyclo[6.6.2]hexadecan-4-yl)acetic acid | |
| CB-TE2A | 4,11-bis-(carboxymethyl)-1,4,8,11-tetraazabicyclo[6.6.2]- | a |
| | hexadecane | |
| CB-TE2P | ((1,4,8,11-tetraazabicyclo[6.6.2]hexadecane-4,11- | a |
| | diyl)bis(methylene))bis(phosphonic acid) | |
| CB-TEAMA | 2-(11-(2-amino-2-oxoethyl)-1,4,8,11- | gg |
| | tetraazabicyclo[6.6.2]hexadecan-4-yl)acetic acid | |
| CB-TR2A | 2,2′-(1,4,8,11-tetraazabicyclo[6.5.2]pentadecane-4,11- | j |
| | diyl)diacetic acid | |
| CDTA | trans-1,2-diaminocyclohexane-N,N,N′,N′-tetraacetic acid | b |
| C-DTPA | bis(2-(2,6-dioxomorpholino)ethyl)glycine | c |
| CHX-A″-DTPA | N-(2-aminoethyl)-trans-1,2-diaminocyclohexane-N,N′,N″- | c |
| | pentaacetic acid | |
| CHX-macropa | rac-6,6′-(((16aS,20aS)/(16aR,20aR)-Hexadecahydro- | c |
| | 4H,13Hbenzo[b][1,4,10,13]tetraoxa[7,16]diazacyclooctadecine- | |
| | 4,13-diyl) bis(methylene))dipicolinic acid | |
| CHX-octapa | 6,6′-((cyclohexane-1,2- | h |
| | diylbis((carboxymethyl)azanediyl))bis(methylene))dipicolinic | |
| | acid | |
| Crown | 2,2′,2″,2′″-1,10-dioxa-4,7,13,16-tetraazacyclooctadecane- | h |
| | 4,7,13,16-tetrayl-tetraacetic acid | |
| Crown-N6₃A | 2,16-dioxo-3,6,9,12,15,21-hexaazabicyclo[15.3.1]henicosa- | c |
| | 1(21),17,19-triene-N(6),N(9),N(12)-triacetic acid | |
| Cyclam | 1,4,8,11-tetraazacyclotetradecane | b |
| Cyclen | 1,4,7,10-tetraazacyclododecane | b |
| DATA(M) | 2,2′-(6-((carboxymethyl)(methyl)amino)-6-methyl-1,4- | m |
| | diazepane-1,4-diyl)diacetic acid | |
| DATA(P) | (2S,2′S)-2,2′-(6-(((S)-1-carboxyethyl)amino)-6-methyl-1,4- | m |
| | diazepane-1,4-diyl)dipropionic acid | |
| DATA(Ph) | d2,2′-(5-((carboxymethyl)amino)-5-phenyldihydropyrimidine- | m |
| | 1,3(2H,4H)-diyl)diacetic acid | |
| DATA(PPh) | (2S,2′S)-2,2′-(5-(((S)-1-carboxyethyl)amino)-5- | m |
| | phenyldihydropyrimidine-1,3(2H,4H)-diyl)dipropionic acid | |
| DE4TA | 2,2′,2″-(10-(2-((carboxymethyl)amino)ethyl)-1,4,7,10- | s |
| | tetraazacyclododecane-1,4,7-triyl)triacetic acid | |
| Deferiprone | 3-hydroxy-1,2-dimethyl-4(1H)-pyridone | e |
| DEPA | 7-[2-(bis-carboxymethylamino)-ethyl]-4,10-bis-carboxymethyl- | c |
| | 1,4,7,10-tetraaza-cyclododec-1-yl-acetic acid | |
| DFC | N-[3-[(2S,5S,8S)-5,8-bis[3-[acetyl(hydroxy)amino]propyl]- | oo |
| | 3,6,9,12,15,18-hexaoxo-1,4,7,10,13,16-hexazacyclooctadec-2- | |
| | yl]propyl]-N-hydroxyacetamide | |
| DFO | N1-(5-aminopentyl)-N1-hydroxy-N4-(5-(N-hydroxy-4-((5-(N- | a |
| | hydroxyacetamido)pentyl)amino)-4- | |
| | oxobutanamido)pentyl)succinamide | |
| DFO-HOPO | N1-hydroxy-N1-(5-(4-(hydroxy(5-(1-hydroxy-6-oxo-1,6- | dd |
| | dihydropyridine-2- carboxamido)pentyl)amino)-4- | |
| | oxobutanamido)pentyl)-N4-(5-(N- | |
| | hydroxyacetamido)pentyl)succinamide | |
| DiAmSar | 1,8-diamino-3,6,10,13,16,19-hexaazabicyclo[6.6.6]icosane | a |
| Diphosphine | 2,3-bis(diphenylphosphaneyl)maleic acid | b |
| DM-TE2A | 2,2′-(4,11-dimethyl-1,4,8,11-tetraazacyclotetradecane-1,8- | a |
| | diyl)diacetic acid | |
| DO2A | 1,4,7,10-tetraazacyclododecane-1,7-diacetic acid | n |
| DO2a2p | 2,2′-(4,10-bis(phosphonomethyl)-1,4,7,10- | n |
| | tetraazacyclododecane-1,7-diyl)diacetic acid | |
| DO2AP | 2,2′-(4-(phosphonomethyl)-1,4,7,10-tetraazacyclododecane-1,7- | o |
| | diyl)diacetic acid | |
| DO2P | ((1,4,7,10-tetraazacyclododecane-1,7- | b |
| | diyl)bis(methylene))bis(phosphonic acid) | |
| DO2PA | 6,6′-((1,4,7,10-tetraazacyclododecane-1,7- | g |
| | diyl)bis(methylene))dipicolinic acid | |
| DO2Py2Am | 1,7-bis(2-pyridylmethyl)-4,10-bis((2-(2-hydroxyethoxy)ethyl)- | c |
| | 2-aminoacetamido)-1,4,7,10-tetraazacyclododecane | |
| DO3A | 1,4,7,10-tetraazacyclododecane-1,4,7-triacetic acid | c |
| DO3A-Bu | 10-(2,3-dihydroxy-(1-hydroxymethyl)propyl)-1,4,7,10- | c |
| | tetraazacyclododecane-1,4,7-triacetic acid | |
| DO3P | ((4-(phosphonomethyl)-1,4,7,10-tetraazacyclododecane-1,7- | b |
| | diyl)bis(methylene))bis(phosphonic acid) | |
| DO3PA | 2-(4,7,10-tris(phosphonomethyl)-1,4,7,10- | n |
| | tetraazacyclododecan-1-yl)acetic acid | |
| DOTA | 1,4,7,10-tetrazacyclododecane-1,4,7,10-tetraacetic acid | a |
| DOTA-2Py | 2,2′-(7,10-bis(pyridin-2-ylmethyl)-1,4,7,10- | h |
| | tetraazacyclododecane-1,4-diyl)diacetic acid | |
| DOTA-3Py | 2-(4,7,10-tris(pyridin-2-ylmethyl)-1,4,7,10- | h |
| | tetraazacyclododecan-1-yl)acetic acid | |
| DOTAGA | 2-(4,7,10-tris(carboxymethyl)-1,4,7,10-tetraazacyclododecan-1- | a |
| | yl)pentanedioic acid | |
| DOTG | 2,2′,2″,2′″-(1,4,7,10-tetraazacyclododecane-1,4,7,10- | s |
| | tetrayl)tetraglutaric acid | |
| DOTHA₂ | 2-(4,7,10-tris(2-(hydroxy(methyl)amino)-2-oxoethyl)-1,4,7,10- | l |
| | tetraazacyclododecan-1-yl)acetic acid | |
| DOTMA | (2R,2′R,2″R,2′″R)-2,2′,2″,2′″-(1,4,7,10-tetraazacyclododecane- | p |
| | 1,4,7,10-tetrayl)tetrapropionic acid | |
| DOTP | ((1,4,7,10-tetraazacyclododecane-1,4,7,10- | c |
| | tetrayl)tetrakis(methylene))tetrakis(phosphonic acid) | |
| DOTPA | 3,3′,3″,3′″-(1,4,7,10-tetraazacyclododecane-1,4,7,10- | s |
| | tetrayl)tetrapropionic acid | |
| DOTPH | 1,4,7,10-tetraazacyclododecane-1,4,7,10- | c |
| | tetrakis(methylenephosphinic acid) | |
| DOTPI | 1,4,7,10-tetraazacyclododecane-1,4,7,10-tetrakis(methylene(2- | c |
| | carboxyethylphosphinic acid)) | |
| DOTP-OEt | 1,4,7,10-tetraazacyclododecane-1,4,7,10- | c |
| | tetrakis(methylene(phosphonic acid monoethyl ester)) | |
| DOTPy/TPC | 1,4,7,10-tetrakis(2-pyridylmethyl)-1,4,7,10- | c |
| | tetraazacyclododecane | |
| DOTPyd | 1,4,7,10-tetrakis(3-pyridazylmethyl)-1,4,7,10- | c |
| | tetraazacyclododecane | |
| DOTPyr | 1,4,7,10-tetrakis(4-pyrimidylmethyl)-1,4,7,10- | c |
| | tetraazacyclododecane | |
| DOTPz | 1,4,7,10-tetrakis(2-pyrazinylmethyl)-1,4,7,10- | c |
| | tetraazacyclododecane | |
| Dp44mT | 2-(di(pyridin-2-yl)methylene)-N,N-dimethylhydrazine-1- | ff |
| | carbothioamide | |
| DpC | di-2-pyridylketone-4-cyclohexyl-4-methyl-3-thiosemicarbazone | mm |
| DTC | diethyldithiocarbamate | nn |
| DTCBP | (3-hydroxy-3,3-diphosphonopropyl)(methyl)carbamodithioic | ll |
| | acid | |
| DTPA | diethylenetriaminepentaacetic acid | a |
| DTPAm | 2,2′,2″,2′″-((((2-amino-2-oxoethyl)azanediyl)bis(ethane-2,1- | i |
| | diyl))bis(azanetriyl))tetraacetamide | |
| ECC | ethylenecysteamine cysteine | s |
| EDTA | ethylenediaminetetraacetic acid | c |
| EDTMP | ethylenediamine-N,N,N′,N″-tetramethylenephosphonic acid | g |
| EGTA | ethylene glycol bis(2-aminoethyl ether)-N,N,N′,N′-tetraacetic | s |
| | acid | |
| FSC | 3,15,27-triamino-7,19,31-trihydroxy-10,22,34-trimethyl- | s |
| | 1,13,25-trioxa-7,19,31-triaza-cyclohexatriaconta-9,21,33-triene- | |
| | 2,8,14,20,26,32-hexaone | |
| H₂ampa | 6,6′-(2,11-bis(2-amino-2-oxoethyl)-5,8-dioxa-2,11- | ss |
| | diazadodecane-1,12-diyl)dipicolinic acid | |
| H₂azapa | 6,6′-((ethane-1,2-diylbis(((1-benzyl-1H-1,2,3-triazol-4- | a |
| | yl)methyl)azanediyl))bis(methylene))dipicolinic acid | |
| H₂BA2A1Py | 2,2′-(7-(pyridin-2-ylmethyl)-2,3,6,7,8,9,11,12-octahydro-4H- | bb |
| | benzo[b][1,4]dioxa[7,10,13]triazacyclopentadecine-4,10(5H)- | |
| | diyl)diacetic acid | |
| H₂Bispa² | 6,6′-((9-hydroxy-1-methoxy-5-(methoxycarbonyl)-2,4- | i |
| | di(pyridin-2-yl)-3,7-diazabicyclo(3.3.1)nonane-3,7- | |
| | diyl)bis(methylene)) dipicolinic acid | |
| H₂CHX-DEDPA | 6,6′-((((1R,2R)-cyclohexane-1,2- | q |
| | diyl)bis(azanediyl))bis(methylene))dipicolinic acid | |
| H₂CHX-macropa | 6,6′-(((16aR,20aR)-hexadecahydro-4H,13H- | i |
| | benzo[b][1,4,10,13]tetraoxa[7,16]diazacyclooctadecine-4,13- | |
| | diyl)bis(methylene))dipicolinic acid | |
| H₂dedpa | 1,2-[[6-(carboxy)-pyridin-2-yl]-methylamino]ethane | a |
| H₂macrodipa | 6,6′-((1,4,7,13-tetraoxa-10,16-diazacyclooctadecane-10,16- | i |
| | diyl)bis(methylene))dipicolinic acid | |
| H₂macropa | N,N′-bis[(6-carboxy-2-pyridyl)methyl]-4,13-diaza-18-crown-6 | i |
| H₂ODO2A | 1-oxa-4,7,10-triazacyclododecane-4,10-diaceticacid | qq |
| H₃BA3A | 2,2′,2″-(5,6,8,9,11,12-hexahydro-2H- | bb |
| | benzo[b][1,4]dioxa[7,10,13]triazacyclopentadecine-4,7,10(3H)- | |
| | triyl)triacetic acid | |
| H₃macrotripa | 6,6′,6″-((1,7,13-trioxa-4,10,16-triazacyclooctadecane-4,10,16- | i |
| | triyl)tris(methylene))tripicolinic acid | |
| H₃THP | 4-amino-N1,N7-bis((3-hydroxy-1,6-dimethyl-4-oxo-1,4- | e |
| | dihydropyridin-2-yl)methyl)-4-(3-(((3-hydroxy-1,6-dimethyl-4- | |
| | oxo-1,4-dihydropyridin-2-yl)methyl)amino)-3- | |
| | oxopropyl)heptanediamide | |
| H₄(3,4,3-(LI-1,2- | N,N′-(butane-1,4-diyl)bis(1-hydroxy-N-(3-(1-hydroxy-6-oxo- | i |
| HOPO)) | 1,6-dihydropyridine-2-carboxamido)propyl)-6-oxo-1,6- | |
| | dihydropyridine-2-carboxamide) | |
| H₄(3,4,3-LI(Me-3,2- | N,N′-(butane-1,4-diyl)bis(3-hydroxy-N-(3-(3-hydroxy-1- | i |
| HOPO)) | methyl-2-oxo-1,2-dihydropyridine-4-carboxamido)propyl)-1- | |
| | methyl-2-oxo-1,2-dihydropyridine-4-carboxamide) | |
| H₄(Me-3,2-HOPO- | 4-((4-(3-(bis(2-(3-hydroxy-1-methyl-2-oxo-1,2- | i |
| OH) | dihydropyridine-4-carboxamido)ethyl)amino)-2-((bis(2-(3- | |
| | hydroxy-1-methyl-2-oxo-1,2-dihydropyridine-4- | |
| | carboxamido)ethyl)amino)methyl)propyl)phenyl)amino)-4- | |
| | oxobutanoic acid | |
| H₄neunpa | 6,6′-(((azanediylbis(ethane-2,1- | i |
| | diyl))bis((carboxymethyl)azanediyl))bis(methylene))dipicolinic | |
| | acid | |
| H₄noneunpa | 6,6′-(((oxybis-(ethane-2,1- | i |
| | diyl))bis((carboxymethyl)azanediyl))bis(methylene))-dipicolinic | |
| | acid | |
| H₄octapa | 6,6′-((ethane-1,2- | a |
| | diylbis((carboxymethyl)azanediyl))bis(methylene))dipicolinic | |
| | acid | |
| H₅decapa | 6,6′-(((((carboxymethyl)azanediyl)bis(ethane-2,1- | a |
| | diyl))bis((carboxymethyl)azanediyl))bis(methylene))dipicolinic | |
| | acid | |
| H₅DO3AP | 1,4,7,10-tetraazacyclododecane-1,4,7-triacetic-10- | i |
| | methylphosphonic acid | |
| H₆phospha | N,N′-(methylenephosphonate)-N,N′-[6- | a |
| | (methoxycarbonyl)pyridin-2-yl]-methyl-1,2-diaminoethane | |
| H₆Sbbpen | N,N′-bis(2-hydroxy-5-sulfonylbenzyl)-N,N′-bis-(2- | s |
| | methylpyridyl) ethylenediamine | |
| H₈(3,4,3-LI(CAM)) | N,N′-(butane-1,4-diyl)bis(N-(3-(2,3- | i |
| | dihydroxybenzamido)propyl)-2,3-dihydroxybenzamide) | |
| H₈(terephthalamide) | N¹,N¹′-((1²,1³,11²,11³-tetrahydroxy-2,10,12,20-tetraoxo- | i |
| | 3,6,9,13,16,19-hexaaza-1,11(1,4)-dibenzenacycloicosaphane- | |
| | 6,16-diyl)bis(ethane-2,1-diyl))bis(2,3-dihydroxy-N⁴-(2- | |
| | methoxyethyl)terephthalamide) | |
| HBED | 2,2′-(ethane-1,2-diylbis((2-hydroxybenzyl)azanediyl))diacetic | a |
| | acid | |
| HBED-CC | 3,3′-(((ethane-1,2- | a |
| | diylbis((carboxymethyl)azanediyl))bis(methylene))bis(4- | |
| | hydroxy-3,1-phenylene))dipropionic acid | |
| Hcb-te1pa | 6-((1,4,8,11-tetraazabicyclo[6.6.2]hexadecan-4-yl)methyl)picolinic | kk |
| | acid | |
| HEHA | 2,2′,2″,2′″,2″″,2′″″-(1,4,7,10,13,16-hexaazacyclooctadecane- | a |
| | 1,4,7,10,13,16-hexayl)hexaacetic acid | |
| HP-DO3A | 10-(2-hydroxypropyl)-1,4,7-tetraazacyclododecane-1,4,7- | r |
| | triacetic acid | |
| HYNIC | 6-hydrazineylnicotinic acid | w |
| LSC (alternative PSC) | 2,2′,2″-(10-(2-amino-2-oxoethyl)-1,4,7,10- | t |
| | tetraazacyclododecane-1,4,7-triyl)triacetic acid | |
| macropaquin | 6-((16-((8-hydroxyquinolin-2-yl)methyl)-1,4,10,13-tetraoxa- | c |
| | 7,16-diazacyclooctadecan-7-yl)methyl)picolinic acid | |
| macrophosphi | (((1,4,10,13-tetraoxa-7,16-diazacyclooctadecane-7,16- | c |
| | diyl)bis(methylene))bis(pyridine-6,2-diyl))bis(methylphosphinic | |
| | acid) | |
| macrophospho | (((1,4,10,13-tetraoxa-7,16-diazacyclooctadecane-7,16- | c |
| | diyl)bis(methylene))bis(pyridine-6,2-diyl))bis(phosphonic acid) | |
| macroquin-SO3 | 8-hydroxy-2-((16-((1-hydroxy-4-sulfoisoquinolin-3-yl)methyl)- | c |
| | 1,4,10,13-tetraoxa-7,16-diazacyclooctadecan-7- | |
| | yl)methyl)quinoline-5-sulfonic acid | |
| MAG3 | (2-mercaptoacetyl)glycylglycylglycine | w |
| MAMA | N-(2-mercaptoethyl)-2-((2-mercaptoethyl)amino)acetamide | w |
| MA-NOTMP | ((1,4,7-triazonane-1,4,7- | y |
| | triyl)tris(methylene))tris(methylphosphinic acid) | |
| Me-DO2Pa | 6,6′-((4,10-dimethyl-1,4,7,10-tetraazacyclododecane-1,7- | c |
| | diyl)bis(methylene))dipicolinic acid | |
| Me-DO2Sc | (2E,2′E)-2,2′-((4,10-dimethyl-1,4,7,10-tetraazacyclododecane- | c |
| | 1,7-diyl)bis(propan-1-yl-2-ylidene))bis(hydrazine-1- | |
| | carboxamide) | |
| MM-TE2A | 2,2′-(4-methyl-1,4,8,11-tetraazacyclotetradecane-1,8- | a |
| | diyl)diacetic acid | |
| MX-DTPA | 2,2′-((1-((2- | g |
| | (bis(carboxymethyl)amino)ethyl)(carboxymethyl)amino)propan- | |
| | 2-yl)azanediyl)diacetic acid | |
| N2S2 | 2,2′-(ethane-1,2-diylbis(azanediyl))bis(ethane-1-thiol) | w |
| N4 | N,N′-bis-(2-amino-ethyl)-propane-1,3-diamine | w |
| N₅3Pa | N,N″-bis(2-aminoethyl)-diethylenetriamino-N,N′,N″-tripicolinic | c |
| | acid | |
| NE3TA | 2,2′-(7-(2-((carboxymethyl)amino)ethyl)-1,4,7-triazonane-1,4- | l |
| | diyl)diacetic acid | |
| NETA | 2,2′-((2-(4,7-bis(carboxymethyl)-1,4,7-triazonan-1- | a |
| | yl)ethyl)azanediyl)diacetic acid | |
| NO2A | 2,2′-(1,4,7-triazonane-1,4-diyl)diacetic acid | s |
| NO2AP | 2,2′-(7-(((2-carboxyethyl)(hydroxy)phosphoryl)methyl)-1,4,7- | s |
| | triazonane-1,4-diyl)diacetic acid | |
| NODAGA | 2-(4,7-bis(carboxymethyl)-1,4,7-triazonan-1-yl)pentanedioic | a |
| | acid | |
| NODA-MPAA | 2,2′-(7-(4-(carboxymethyl)benzyl)-1,4,7-triazonane-1,4- | tt |
| | diyl)diacetic acid | |
| NODASA | 2-(4,7-bis(carboxymethyl)-1,4,7-triazonan-1-yl)succinic acid | s |
| NOPO | 3-(((4,7-bis((hydroxy(hydroxymethyl)phosphoryl)methyl)- | a |
| | 1,4,7-triazonan-1-yl)methyl)(hydroxy)phosphoryl)propanoic | |
| | acid | |
| NOTA | 1,4,7-triazacyclononane- N,N′,N″-triacetic acid | a |
| NOTAM | 2,2′,2″-(1,4,7-triazonane-1,4,7-triyl)triacetamide | z |
| NOTHA₂ | 2-(4,7-bis(2-(hydroxy(methyl)amino)-2-oxoethyl)-1,4,7- | l |
| | triazonan-1-yl)acetic acid | |
| NOTP | ((1,4,7-triazonane-1,4,7-triyl)tris(methylene))tris(phosphonic | s |
| | acid) | |
| NPTA | ((4-carboxymethyl-7-(2-(carboxymethylamino)-ethyl)-perhydro- | c |
| | 1,4,7-triazonin-1-yl)-acetic acid | |
| NxS4-x | stands for a group of tetradentate chelators with N-atoms (basic | w |
| | amine or non-basic amide) and thiols as donors stabilizing Tc- | |
| | complexes, especially Tc(V)-oxo complexes | |
| Oxabiphore | (((oxybis(ethane-2,1-diyl))bis(azanetriyl))tetrakis(methylene)) | g |
| | tetrakis(phosphonic acid) | |
| oxo-DO3A | 2,2′,2″-(1-oxa-4,7,10-triazacyclododecane-4,7,10-triyl)triacetic | a |
| | acid | |
| PCBA | 2-((1,4,7,10,13-pentaazacyclopentadecan-1-yl)methyl)benzoic | ee |
| | acid | |
| PCB-TE2A | 2,2′-(1,4,8,11-tetraazabicyclo[6.6.3]heptadecane-4,11- | k |
| | diyl)diacetic acid | |
| PCTA | 2,2′,2″-(3,6,9-triaza-1(2,6)-pyridinacyclodecaphane-3,6,9- | a |
| | triyl)triacetic acid | |
| p-EDDHA | 2,2′-(ethane-1,2-diylbis(azanediyl))bis(2-(4- | w |
| | hydroxyphenyl)acetic acid) | |
| PEPA | 2,2′,2″,2′″,2″″-(1,4,7,10,13-pentaazacyclopentadecane- | a |
| | 1,4,7,10,13-pentayl)pentaacetic acid | |
| PIH | (E)-N′-((3-hydroxy-5-(hydroxymethyl)-2-methylpyridin-4- | ff |
| | yl)methylene)isonicotinohydrazide | |
| PIP-DOTA | cis-((1R,11S)-6,9,15-tris-car-boxymethyl-3,6,9,15- | c |
| | tetraazabicyclo[9.3.1]pentadec-3-yl)-acetic acid | |
| PIP-DTPA | cis-2,6-bis(N,N-bis(carboxymethyl)aminomethyl)-1-piperidine | c |
| | acetic acid | |
| PSC (alternative LSC) | 2,2′,2″-(10-(2-amino-2-oxoethyl)-1,4,7,10- | t |
| | tetraazacyclododecane-1,4,7-triyl)triacetic acid | |
| PTSM | Pyruvaldehyde bis(N⁴-methylthiosemicarbazone) | b |
| Py4pa | 6,6′-(((pyridine-2,6-diylbis(methylene))- | h |
| | bis((carboxymethyl)azanediyl))bis(methylene))dipicolinic acid | |
| Pycup2A | 1,8-(2,6-Pyridinedimethylene)-1,4,8,11-tetraazacyclotetra- | l |
| | decane-2,4-diacetic acid | |
| QT | quinoline thiosemicarbazone | rr |
| Sarcophagine | 3,6,10,13,16,19-hexazabicyclo[6.6.6]icosane | b |
| SHBED | 2,2′-(ethane-1,2-diylbis((2-hydroxy-5- | a |
| | sulfobenzyl)azanediyl))diacetic acid | |
| Tachpyr | N1,N3,N5-tris(pyridin-2-ylmethyl)cyclohexane-1,3,5-triamine | b |
| TACN | 1,4,7-triazonane | v |
| TACN-TM | tris(2-mercaptoethyl)-1,4,7-triazacyclononane | a |
| TAM | 2,3-dihydroxyterephthalamide | u |
| TAME | 2-(aminomethyl)-2-methylpropane-1,3-diamine | v |
| TAME-Hex | (1,8-N,N′-bis(carboxymethyl)-1,4,8,11-tetraazacyclotetradecane | v |
| TCMC | 2,2′,2″,2′″-(1,4,7,10-tetraazacyclododecane-1,4,7,10- | a |
| | tetrayl)tetraacetamide | |
| TE2A | 2,2′-(1,4,8,11-tetraazacyclotetradecane-1,8-diyl)diacetic acid | a |
| TE2Py2Am | 1,8-bis(2-pyridylmethyl)-4,11-bis((2-(2-hydroxyethoxy)ethyl)- | a |
| | 2-aminoacetamido)-1,4,8,11-tetraaza- cyclotetradecane | |
| TE3A | 2,2′,2″-(1,4,8,11-tetraazacyclotetradecane-1,4,8-triyl)triacetic | a |
| | acid | |
| TETA | 2,2′,2″,2′″-(1,4,8,11-tetraazacyclotetradecane-1,4,8,11- | a |
| | tetrayl)tetraacetic acid | |
| tet-a | (7S,14R)-5,5,7,12,12,14-hexamethyl-1,4,8,11- | jj |
| | tetraazacyclotetradecane | |
| TETAM | 2,2′,2″,2′″-(1,4,8,11-tetraazacyclotetradecane-1,4,8,11- | pp |
| | tetrayl)tetraacetamide | |
| TETPA | 3,3′-(4,11-bis(carboxymethyl)-1,4,8,11- | s |
| | tetraazacyclotetradecane-1,8-diyl)dipropionic acid | |
| THPN | 1,3-propanediamine-N,N,N′,N′-tetrakis[(2-(aminomethyl)-3- | oo |
| | hydroxy-1,6-dimethyl-4(1H)-pyridinone)acetamide] | |
| TMT-amine | 2,2′,2″,2′″-(((4′-(3-amino-4-methoxyphenyl)-[2,2′:6′,2″- | hh |
| | terpyridine]-6,6″-diyl)bis(methylene))bis(azanetriyl))tetraacetic | |
| | acid | |
| TRAP | 3,3′,3″-(((1,4,7-triazonane-1,4,7- | a |
| | triyl)tris(methylene))tris(hydroxyphosphoryl))tripropanoic acid | |
| TREN(Me-3,2-HOPO) | N,N′-(((2-(3-hydroxy-2-oxo-1,2-dihydropyridine-4- | ii |
| | carboxamido)ethyl)azanediyl)bis(ethane-2,1-diyl))bis(3- | |
| | hydroxy-1-methyl-2-oxo-1,2-dihydropyridine-4-carboxamide) | |
| Triapine | (E)-2-((3-aminopyridin-2-yl)methylene)hydrazine-1- | ff |
| | carbothioamide | |
| TRITA | 2,2′,2″,2′″-(1,4,7,10-tetraazacyclotridecane-1,4,7,10- | s |
| | tetrayl)tetraacetic acid | |
| TRITAM | 2,2′,2″,2′″-(1,4,7,10-tetraazacyclotridecane-1,4,7,10- | pp |
| | tetrayl)tetraacetamide | |
| TTHA | triethylenetetramine-N,N,N′,N″,N′″,N′″-hexaacetic acid | s |
| [16]aneS₄ | 1,5,9,13-tetrathiacyclohexadecane | cc |
| [2,2,2]-cryptand | 4,7,13,16,21,24-hexaoxa-1,10-diazabicyclo(8.8.8) hexacosane | x |
| 6SS | N,N′-bis(2,2-dimethyl-2-mercaptoethyl)ethylenediamine-N,N′- | s |
| | diacetic acid | |
| ^{99m}Tc(CO)₃-Chelators | Ligand platform that accommodates the ^{99m}Tc(CO)₃-core | w |

a) Price, et al., Chem Soc Rev, 2014, 43: 260.
b) Wadas et al., Curr. Pharm. Des. 2007, 13 (1)
c) Franchi et al., Nucl. Med. Biol. 2022, 114-115, 168
d) Allott et al., Chem Commun (Camb) 2017, 53, 8529
e) Cusnir et al., Int J Mol Sci 2017, 18
f) Demoin, et al., Nucl Med Biol 2016, 43: 802
g) Egorova et al., Russ. Chem. Rev. 2019, 88 (9), 901
h) Yang et al., J. Nucl. Med. 2022, 63(1), 5
i) Grieve et al., Aust. J. Chem. 2022, 75, 65
j) Odendaal et al., Inorg Chem. 2011, 50(7), 3078
k) Pandya et al., Bioconjugate Chem. 2012, 23(3), 330
l) Gai et al., Inorg Chem. 2016, 55(14), 6892
m) Seemann et al., ChemMedChem 2015, 10(6), 1019
n) Kálmán et al., Inorg. Chem. 2008, 47(9), 3851
o) Travagin et al., Synlett 2020, 31, 1291
p) Woods et al., Chemistry 2019, 25(42), 9997
q) Ramogida et al., Inorg. Chem. 2015, 54, 2017
r) Barge et al., Org. Biomol. Chem. 2009, 7, 3810
s) Kostelnik et al., Chem. Rev. 2019, 119(2), 902
t) Li et al., Pharmaceutics 2023, 15, 414
u) McAuley, et al., Can. J. Chem. 1989, 67, 1657
v) Maecke et al. in: PET Chemistry (Springer Berlin Heidelberg) 2007, 64, 215
w) Mazzi et al. in: Technetium-99m pharmaceuticals (Berlin: Springer) 2007, 7-58
x) McDonagh et al., Inorg. Chem. 2021, 60, 10030
y) Poty et al., ChemMedChem 2015, 10(9), 1475
z) AlHokbany et al., EJNMMI Radiopharm Chem. 2022, 7(19)
aa) Urbanovsky et al., RSC Adv. 2020, 10, 21329
bb) Egorova et al., Molecules 2022, 27, 3115
cc) Straathof et al., Molecules 2021, 26, 4603
dd) Yu et al., J. Inorg. Biochem. 2016, 154, 89
ee) Lozza et al., Front. Oncol. 2013, 3, 247
ff) Heath et al., Nutrients 2013, 5(8), 2836
gg) Wadas et al., Chem. Rev. 2010, 110(5), 2858
hh) Stimmel et al., Bioconjugate Chem. 1995, 6(2), 219
ii) Yokel et al., J. Pharm. Sci. 2000, 89(4), 545
jj) Woodin et al., Eur. J. Inorg. Chem. 2005, 4829
kk) Lima et al., Inorg. Chem. 2014, 53(10), 5269
ll) Cai et al., J. Label Compd. Radiopharm 2014, 57, 224
mm) Guo et al., J Hematol Oncol. 2016, 9, 98
nn) Kanchi et al., Arab. J. Chem. 2014, 7(1), 11
oo) Feiner et al., Cancers 2021, 13, 4466
pp) Lyczko et al., Polyhedron 2020, 192, 114822
qq) Chang et al., Dalton Trans., 2013, 42, 6397
rr) Serda et al., PLOS ONE 2014, 9(10), e110291
ss) Ingham et al., Inorg. Chem. 2022, 61(24), 9119
tt) McBride et al., EJNMMI Research 2013, 3(36)

HYNIC, DTPA, EDTA, DOTA, TETA, and bisamino bisthiol (BAT)-based chelators are disclosed in U.S. Pat. No. 5,720,934; desferrioxamine (DFO) is disclosed in Doulias et al. (Doulias, et al., Free Radic Biol Med, 2003, 35: 719); tetrapyridine and N3S, N2S2 and N4 chelators are disclosed in U.S. Pat. Nos. 5,367,080 A, 5,364,613 A, 5,021,556 A, 5,075,099 A, 5,886,142 A, whereby all of the references are included herein by reference in their entirety. 6-amino-6-methylperhydro-1,4-diazepine-N,N′,N″,N″-tetraacetic acid (AAZTA) is disclosed in Pfister et al. (Pfister, et al., EJNMMI Res, 2015, 5: 74); deferiprone, a 1,2-dimethyl-3,4-hydroxypyridinone and hexadentate tris(3,4-hydroxypyridinone) (THP) are disclosed in Cusnir et al. (Cusnir, et al., Int J Mol Sci, 2017, 18); monoamine-monoamide dithiol (MAMA)-based chelators are disclosed in Demoin et al. (Demoin, et al., Nucl Med Biol, 2016, 43: 802); Macropa and analogs are disclosed in Thiele et al. (Thiele, et al., Angew Chem Int Ed Engl, 2017, 56: 14712); 1,4,7,10,13,16-hexaazacyclohexadecane-N,N′,N″,N′″,N″″,N′″″-hexaacetic acid (HEHA) and PEPA analogs are disclosed in Price and Orvig (Price, et al., Chem Soc Rev, 2014, 43: 260); pycup and analogs are disclosed in Boros et al. (Boros, et al., Mol Pharm, 2014, 11: 617) N, N-bis(2-hydroxybenzyl)ethylenediamine-N,N-diacetic acid (HBED), 1,4,7,10-tetrakis(carbamoylmethyl)-1,4,7,10-tetraazacyclododecane (TCM), 2-[(carboxymethyl)]-[5-(4-nitrophenyl-1-[4,7,10-tris-(carboxymethyl)-1,4,7,10-tetraazacyclododecan-1-yl]pentan-2-yl)-amino]acetic acid (3p-C-DEPA), CB-TE2A, TE2A, TE1A1P, DiAmSar, 1-N-(4-aminobenzyl)-3,6,10,13,16,19-hexaazabicyclo[6.6.6]-eicosane-1,8-diamine (SarAr), NETA, tris(2-mercaptoethyl)-1,4,7-triazacyclononane (TACN-TM), {4-[2-(bis-carboxymethyl-amino)-ethyl]-7-carboxymethyl-[1,4,7]triazonan-1-yl}-acetic acid (NETA), diethylenetriaminepentaacetic acid (DTP), 3-({4,7-bis-[(2-carboxy-ethyl)-hydroxy-phosphinoylmethyl]-[1,4,7]triazonan-1-ylmethyl}-hydroxy-phosphinoyl)-propionic acid (TRAP), NOPO, H4octapa, SHBED, BPCA, 3,6,9,15-tetraazabicyclo[9.3.1]-pentadeca-1(15),11,13-triene-3,6,9-triacetic acid (PCTA), and 1,4,7,10,13-pentaazacyclopentadecane-N,N′,N″,N′″,N″″-pentaacetic acid (PEPA) are disclosed in Price and Orvig (Price, et al., Chem Soc Rev, 2014, 43: 260); 1-hydroxy-2-pyridone ligand (HOPO) is disclosed in Allott et al. (Allott, et al., Chem Commun (Camb), 2017, 53: 8529); [4-carboxymethyl-6-(carboxymethyl-methyl-amino)-6-methyl-[1,4]diazepan-1-yl]-acetic acid (DATA) is disclosed in Tornesello et al. (Tornesello, et al., Molecules, 2017, 22: 1282); tetrakis(aminomethyl)methane (TAM) and analogs are disclosed in McAuley 1988 (McAuley, et al., Canadian Journal of Chemistry, 1989, 67: 1657); hexadentate tris(3,4-hydroxypyridinone) (THP) and analogs are disclosed in Ma et al. (Ma, et al., Dalton Trans, 2015, 44: 4884).

The diagnostic and/or therapeutic use of some of the above chelators is described in the prior art. For example, 2-hydrazino nicotinamide (HYNIC) has been widely used in the presence of a coligand for incorporation of ^{99m}Tc and ^{186,188}Re (Schwartz, et al., Bioconjug Chem, 1991, 2: 333; Babich, et al., J Nucl Med, 1993, 34: 1964; Babich, et al., Nucl Med Biol, 1995, 22: 25); DTPA is used in Octreoscan® for complexing ¹¹¹In and several modifications are described in the literature (Li, et al., Nucl Med Biol, 2001, 28: 145; Brechbiel, et al., Bioconjug Chem, 1991, 2: 187); DOTA-type chelators for radiotherapy applications are described by Tweedle et al. (U.S. Pat. No. 4,885,363); other polyaza macrocycles for chelating trivalent isotopes metals are described by Eisenwiener et al. (Eisenwiener, et al., Bioconjug Chem, 2002, 13: 530); and N4-chelators such as a ^{99m}Tc-N4-chelator have been used for peptide labeling in the case of minigastrin for targeting CCK-2 receptors (Nock, et al., J Nucl Med, 2005, 46: 1727).

In an embodiment the chelator is selected from the group, but not limited to, comprising DOTA, DOTAGA, DOTAM, Crown, DOTP, NOTA, NODAGA, NODA-MPAA, HBED, TETA, CB-TE2A, DTPA, CHX-A″-DTPA, DFO, Macropa, HOPO, TRAP, THP, DATA, NOPO, NOTP, PCTA, LSC (alternative: PSC), sarcophagine, FSC, NETA, NE3TA, H4octapa, pycup, HYNIC, NxS4-x (N4, N2S2, N3S), ^{99m}Tc(CO)₃-chelators and their analogs. Preferably, the chelator additionally comprises one or more functional groups or functionalities allowing attachment to the compound of the invention.

The chemical structures thereof being as follows:

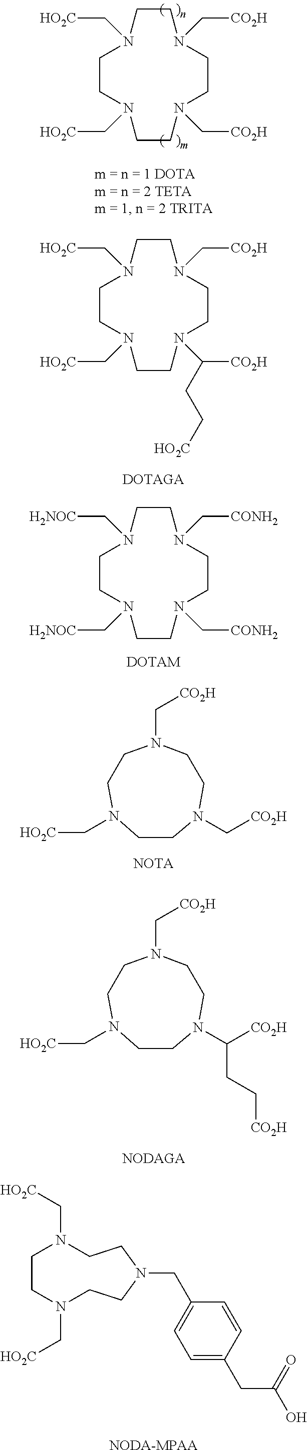

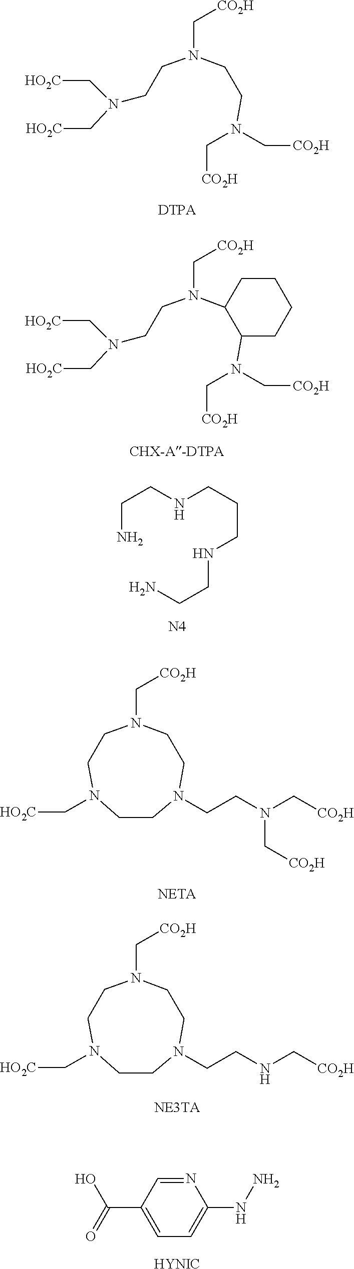

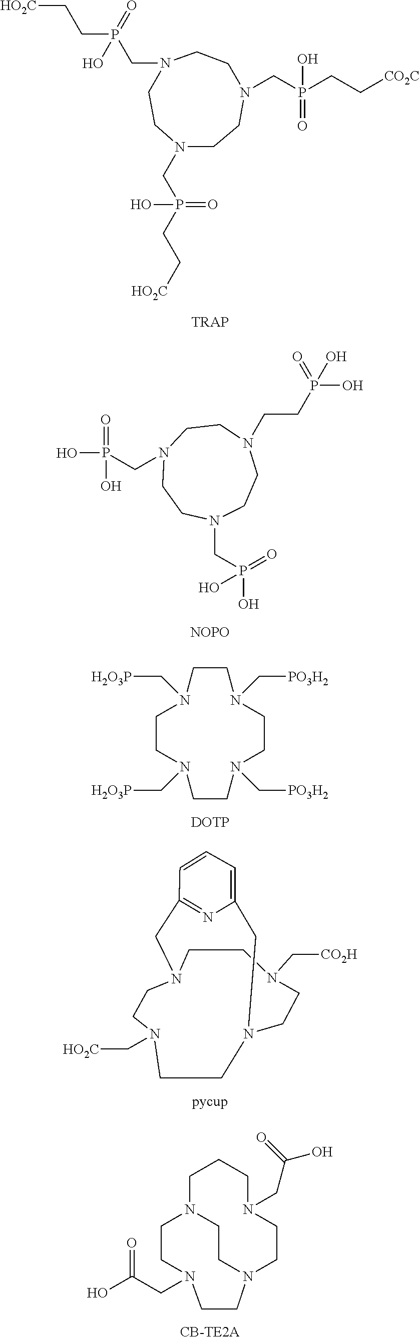

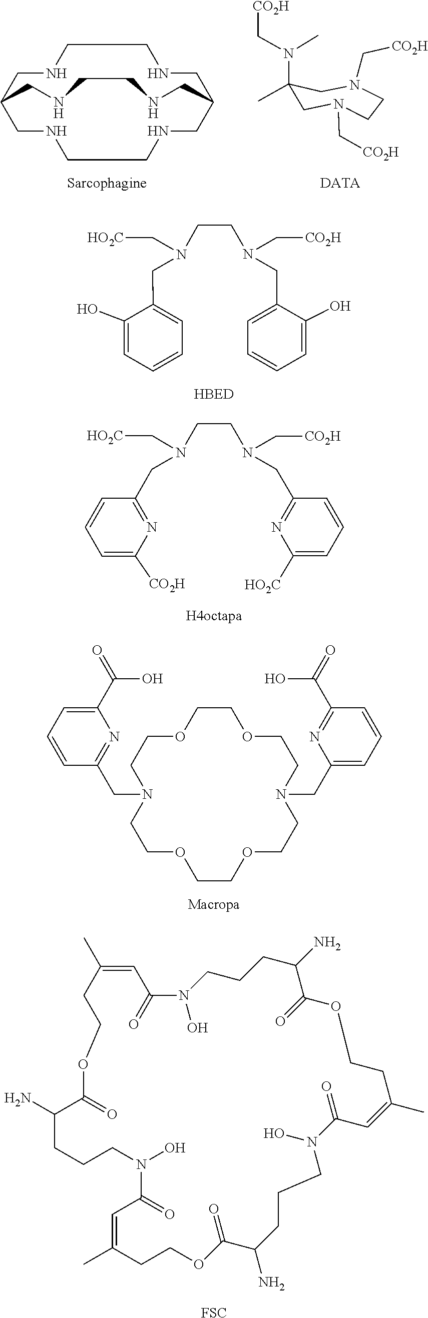

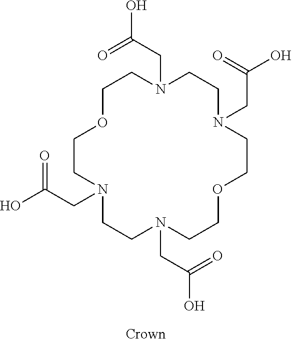 [SMILES: O=C(O)CN1CCOCCN(CC(=O)O)CCN(CC(=O)O)CCOCCN(CC(=O)O)CC1]

In one embodiment, the chelator is selected from the group consisting of DOTA, DOTAGA, NOPO, PCTA, NOTA, NODAGA, NODA-MPAA, HBED, TETA, CB-TE2A, DTPA, DFO, Macropa, Crown, DOTAM, HOPO, TRAP, THP, DATA, NOTP, LSC (alternative PSC), sarcophagine, FSC, NETA, H4octapa, Pycup, NₓS₄₋ₓ (N4, N2S2, N3S), Hynic, ^{99m}Tc(CO)₃-Chelators.

In another embodiment, the chelator is selected from the group consisting of DOTA, DOTAGA, NOPO, PCTA, DOTAM, Macropa, Crown, NOTA, NODAGA, NODA-MPAA, HBED, CB-TE2A, DFO, THP, LSC (alternative PSC) and N4.

In another embodiment, the chelator is selected from the group consisting of DOTA, DOTAGA, NOPO, PCTA, DOTAM, Macropa, Crown, NOTA, LSC (alternative PSC) and NODAGA.

In another embodiment, the chelator is selected from the group consisting of DOTA, DOTAM, Macropa, Crown, NOTA, LSC (alternative PSC) and NODAGA.

In another embodiment, the chelator is DOTA.

Preferably, the chelator additionally comprises one or more functional groups or functionalities allowing attachment to the compounds of the invention.

It will be acknowledged by the persons skilled in the art that the chelator, in principle, may be used regardless whether the compound of the invention is used in or suitable for diagnosis or therapy. Such principle is, among others, outlined in international patent application WO 2009/109332 A1.

It will be further acknowledged by the persons skilled in the art that the presence of a chelator in the compound of the invention includes, if not stated otherwise, the possibility that the chelator is complexed to any metal complex partner, i.e. any metal which, in principle, can be complexed by the chelator. An explicitly mentioned chelator of a compound of the invention or the general term chelator in connection with the compound of the invention refers either to the uncomplexed chelator as such or to the chelator to which any metal complex partner is bound, wherein the metal complex partner is any radioactive or non-radioactive metal complex partner. Preferably the chelator-metal complex, i.e. the chelator to which the metal complex partner is bound, is a stable chelator-metal complex.

Non-radioactive chelator-metal complexes have several applications, e.g., for assessing properties like stability or activity which are otherwise difficult to determine. One aspect is that cold variants of the radioactive versions of the metal complex partner (e.g., non-radioactive indium complexes as described in the examples) can act as surrogates of the radioactive compounds. Furthermore, they are valuable tools for identifying metabolites in vitro or in vivo, as well as for assessing toxicity properties of the compounds of invention. Additionally, chelator-metal complexes can be used in binding assays utilizing the fluorescence properties of some metal complexes with distinct ligands (e.g., Europium salts).

Chelators can be synthesized or are commercially available with a wide variety of (possibly already activated) groups for the conjugation to peptides or amino acids.

Direct conjugation of a chelator to an amino-nitrogen of the respective compound of invention is well possible for chelators selected from the group consisting of DTPA, DOTA, DOTAGA, NOTA, NODAGA, NODA-MPAA, HBED, TETA, CB-TE2A, DFO, DATA, sarcophagine and N4, preferably DTPA, DOTA, DOTAGA, NOTA, NODAGA, NODA-MPAA, CB-TE2A, and N4. The preferred linkage in this respect is an amide linkage.

Direct conjugation of an isothiocyanate-functionalized chelator to an amino-nitrogen of the respective compound of invention is well possible for chelators selected from the group consisting of DOTA, DOTAGA, NOTA, NODAGA, DTPA, CHX-A″-DTPA, DFO, and THP, preferably DOTA, DOTAGA, NOTA, NODAGA, DTPA, and CHX-A″-DTPA. The preferred linkage in this respect is a thiourea linkage.

Functional groups at a chelator which are preferred precursors for the direct conjugation of a chelator to an amino-nitrogen are known to the person skilled in the art and include but are not limited to carboxylic acid, activated carboxylic acid, e.g., active ester like for instance NHS-ester, pentafluorophenol-ester, HOBt-ester, HOAt-ester, and isothiocyanate.

Functional groups at a chelator which are preferred precursors for the direct conjugation of a chelator to a carboxylic group are known to the person skilled in the art and include but are not limited to alkylamino and arylamino nitrogens. Respective chelator reagents are commercially available for some chelators, e.g., for DOTA with either alkylamino or arylamino nitrogen.

Functional groups at a chelator which are preferred precursors for the direct conjugation of a chelator to a thiol group are known to the person skilled in the art and include but are not limited to maleimide nitrogens. Respective chelator reagents are commercially available for some chelators, e.g., for DOTA with maleimide nitrogen.

Functional groups at a chelator which are preferred precursors for the direct conjugation of a chelator to an azide group are known to the person skilled in the art and include but are not limited to acyclic and cyclic alkynes. Respective chelator reagents are commercially available for some chelators, e.g., for DOTA with propargyl or butynyl.

Functional groups at a chelator which are preferred precursors for the direct conjugation of a chelator to an alkyne group are known to the person skilled in the art and include but are not limited to alkyl and aryl azines. Respective chelator reagents are commercially available for some chelators, e.g., for DOTA with azidopropyl.

It will be acknowledged by a person skilled in the art that the radioactive nuclide which is or which is to be attached to the compound of the disclosure, is selected taking into consideration the disease to be treated and/or the disease to be diagnosed, respectively, and/or the particularities of the patient and patient group, respectively, to be treated and to be diagnosed, respectively.

In the present disclosure, a radioactive nuclide is also referred to as radionuclide. Radioactive decay is the process by which an atomic nucleus of an unstable atom loses energy by emitting ionizing particles (ionizing radiation). There are different types of radioactive decay. A decay, or loss of energy, results when an atom with one type of nucleus, called the parent radionuclide, transforms to an atom with a nucleus in a different state, or to a different nucleus containing different numbers of protons and neutrons. Either of these products is named the daughter nuclide. In some decays the parent and daughter are different chemical elements, and thus the decay process results in nuclear transmutation (creation of an atom of a new element). For example, the radioactive decay can be alpha decay, beta decay, and gamma decay. Alpha decay occurs when the nucleus ejects an alpha particle (helium nucleus). This is the most common process of emitting nucleons, but in rarer types of decays, nuclei can eject protons, or specific nuclei of other elements (in the process called cluster decay). Beta decay occurs when the nucleus emits an electron (β⁻-decay) or positron (β⁺-decay) and a type of neutrino, in a process that changes a proton to a neutron or the other way around. By contrast, there exist radioactive decay processes that do not result in transmutation. The energy of an excited nucleus may be emitted as a gamma ray in gamma decay, or used to eject an orbital electron by interaction with the excited nucleus in a process called internal conversion, or used to absorb an inner atomic electron from the electron shell whereby the change of a nuclear proton to neutron causes the emission of an electron neutrino in a process called electron capture (EC), or may be emitted without changing its number of proton and neutrons in a process called isomeric transition (IT). Another form of radioactive decay, the spontaneous fission (SF), is found only in very heavy chemical elements resulting in a spontaneous breakdown into smaller nuclei and a few isolated nuclear particles.

In an embodiment, described herein are compounds that comprise a radionuclide. Generally, the type of radionuclide used in a therapeutic radiopharmaceutical can be tailored to the specific type of cancer and the type of targeting moiety. Radionuclides that undergo α-decay produce particles composed of two neutrons and two protons, and radionuclides that undergo β-decay emit energetic electrons from their nuclei. Some radionuclides can also emit Auger electrons. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate.

Radionuclides that are α-emitters are capable of destroying tumors while causing very limited damage to the surrounding healthy tissue due to the short penetration depth of a particles. Their high linear energy transfer (LET) gives them an increased relative biological effectiveness (RBE) as compared to other radionuclide therapies. Furthermore, when α-emitting radionuclides are targeted to specific tumor cells in the body, they can be very effective in destroying metastases, which are difficult to treat by currently employed techniques (de Kruijff et al, Pharmaceuticals, 2015, 8:, 321-336).

In an embodiment of the present disclosure, the radionuclide can be used for labeling of the compound of the disclosure.

In an embodiment of the present disclosure, the radionuclide is suitable for complexing with a chelator, leading to a radionuclide chelate complex.

In a further embodiment one or more atoms of the compound of the disclosure are of non-natural isotopic composition, for example these atoms are radionuclides; for example radionuclides of carbon, oxygen, nitrogen, sulfur, phosphorus and halogens. These radioactive atoms are typically part of amino acids, in some case halogen containing amino acids, and/or building blocks and in some cases halogenated building blocks each of the compound of the disclosure.

In one embodiment of the present disclosure, the radionuclide has a half-life that allows for diagnostic and/or therapeutic medical use. Specifically, the half-life is between 1 min and 100 days.

In an embodiment of the present disclosure, the radionuclide has a decay energy that allows for diagnostic and/or therapeutic medical use. Specifically, for γ-emitting isotopes, the decay energy is between 0.004 and 10 MeV, for example, between 0.05 and 4 MeV, for diagnostic use. For positron-emitting isotopes, the decay energy is between 0.6 and 13.2 MeV, for example, between 1 and 6 MeV, for diagnostic use. For particle-emitting isotopes, the decay energy is between 0.039 and 10 MeV, for example, between 0.4 and 6.5 MeV, for therapeutic use.

In an embodiment of the present invention, the radionuclide is industrially produced for medical use. Specifically, the radionuclide is available in GMP quality.

In an embodiment of the present disclosure, the daughter nuclide(s) after radioactive decay of the radionuclide are compatible with the diagnostic and/or therapeutic medical use.

Furthermore, the daughter nuclides are either stable or further decay in a way that does not interfere with, or may even support, the diagnostic and/or therapeutic medical use.

Representative radionuclides, which may be used in connection with the present disclosure are well known to the person skilled in the art and include, but are not limited, to the following ones: ¹¹C, ¹³N, ¹⁸F, ²⁴Na, ²⁸Mg, ³¹Si, ³²P, ³³P, ³⁸S, ^{34m}Cl, ³⁸Cl, ³⁹Cl, ³⁷Ar, ⁴¹Ar, ⁴⁴Ar, ⁴²K, ⁴³K, ⁴⁴K, ⁴⁵K, ⁴⁷Ca, ⁴³Sc, ⁴⁴Sc, ^{44m}Sc, ⁴⁷Sc, ⁴⁸Sc, ⁴⁹Sc, ⁴⁵Ti, ⁴⁷V, ⁴⁸V, ⁴⁸Cr, ⁴⁹Cr, ⁵¹Cr, ⁵¹Mn, ⁵²Mn, ^{52m}Mn, ⁵⁶Mn, ⁵²Fe, ⁵⁹Fe, ⁵⁵Co, ⁶¹Co, ^{62m}Co, ⁵⁶Ni, ⁵⁷Ni, ⁶⁵Ni, ⁶⁶Ni, ⁶⁰Cu, ⁶¹Cu, ⁶⁴Cu, ⁶⁷Cu, ⁶²Zn, ⁶³Zn, ⁶⁹Zn, ^{69m}Zn, ^{71m}Zn, ⁷²Zn, ⁶⁵Ga, ⁶⁶Ga, ⁶⁷Ga, ⁶⁸Ga, ⁷⁰Ga, ⁷²Ga, ⁷³Ga, ⁶⁶Ge, ⁶⁷Ge, ⁶⁹Ge, ⁷¹Ge, ⁷⁵Ge, ⁷⁷Ge, ⁷⁸Ge, ⁶⁹As, ⁷⁰As, ⁷¹As, ⁷²As, ⁷⁴As, ⁷⁶As, ⁷⁷As, ⁷⁸As, ⁷⁰Se, ⁷²Se, ⁷³Se, ^{73m}Se, ⁸¹Se, ^{81m}Se, ⁸³S, ⁷⁴Br, ^{74m}Br, ⁷⁵Br, ⁷⁶Br, ⁷⁷Br, ⁸⁰Br, ^{80m}Br, ⁸²Br, ⁸³Br, ⁸⁴Br, ⁷⁴Kr, ⁷⁶Kr, ⁷⁷Kr, ⁷⁹Kr, ⁸⁵Kr, ⁸⁷Kr, ⁸⁸Kr, ⁷⁸Rb, ⁷⁹Rb, ⁸¹Rb, ⁸²Rb, ⁸⁴Rb, ^{84m}Rb, ⁸⁶Rb, ⁸⁸Rb, ⁸⁹Rb, ⁸⁰Sr, ⁸¹Sr, ⁸²Sr, ⁸³Sr, ^{85m}Sr, ⁸⁷Sr, ⁹¹Sr, ⁹²Sr, ⁸⁴Y, ⁸⁵Y, ^{85m}Y, ⁸⁶Y, ^{86m}Y, ⁸⁷Y, ^{87m}Y, ⁹⁰Y, ^{90m}Y, ^{91m}Y, ⁹²Y, ⁹³Y, ⁹⁴Y, ⁹⁵Y, ⁸⁶Zr, ⁸⁷Zr, ⁸⁹Zr, ⁹⁷Zr, ⁸⁸N, ⁸⁹N, ^{89m}N, ⁹⁰Nb, ⁹²Nb, ⁹⁵Nb, ^{95m}Nb, ⁹⁶Nb, ⁹⁷Nb, ^{98m}Nb, ¹⁰¹Mo, ¹⁰²Mo, ⁹⁰Mo, ⁹¹Mo, ^{93m}Mo, ⁹⁹Mo, ¹⁰¹Tc, ¹⁰⁴Tc, ⁹³Tc, ^{93m}Tc, ⁹⁴Tc, ^{94m}Tc, ⁹⁵Tc, ⁹⁶Tc, ^{99m}Tc, ¹⁰³Ru, ¹⁰⁵Ru, ⁹⁴Ru, ⁹⁵Ru, ⁹⁷Ru, h, ¹⁰⁰Rh, ^{101m}Rh, ¹⁰⁵Rh, ^{106m}Rh, ¹⁰⁷Rh, ⁹⁷Rh, ^{97m}Rh, ⁹⁹Rh, ^{99m}Rh, ¹⁰⁰Pd, ¹⁰¹Pd, ¹⁰³Pd, ¹⁰⁹Pd, ¹¹¹Pd, ^{111m}Pd, ¹¹²Pd, ⁹⁸Pd, ⁹⁹Pd, ¹⁰¹Ag, ¹⁰³Ag, ¹⁰⁴Ag, ^{104m}Ag, ¹⁰⁵Ag, ¹⁰⁶Ag, ^{106m}Ag, ¹¹¹Ag, ¹¹²Ag, ¹¹³Ag, ¹¹⁵Ag, ¹⁰⁴Cd, ¹⁰⁵Cd, ¹⁰⁷Cd, ¹¹¹Cd, ¹¹⁵Cd, ^{115m}Cd, ¹¹⁷Cd, ^{117m}Cd, ¹¹⁸Cd, ¹⁰⁷In, ^{108m}In, ¹⁰⁹In, ¹¹⁰In, ^{110m}In, ¹¹¹In, ¹¹²In, ¹¹³In, ^{114m}In, ^{115m}In, ^{116m}In, ¹¹⁷In, ^{117m}In, ^{119m}In, ¹⁰⁸Sn, ¹⁰⁹Sn, ¹¹⁰Sn, ¹¹¹Sn, ¹¹⁷Sn, ¹²¹Sn, ^{123m}Sn, ¹²⁵Sn, ¹²⁷Sn, ¹²⁸Sn, ¹¹⁵Sb, ¹¹⁶Sb, ^{116m}Sb, ¹¹⁷Sb, ^{118m}Sb, ¹¹⁹Sb, ¹²⁰Sb, ^{120m}Sb, ¹²²Sb, ¹²⁶Sb, ^{126m}Sb, ¹²⁷Sb, ¹²⁸Sb, ^{128m}Sb, ¹²⁹Sb, ^{129m}Sb, ¹³⁰Sb, ¹³¹Sb, ¹¹⁴Te, ¹¹⁶Te, ¹¹⁷Te, ¹¹⁸Te, ¹¹⁹Te, ^{119m}Te, ¹²¹Te, ¹²⁷Te, ¹²⁹Te, ^{129m}Te, ¹³¹Te, ^{131m}Te, ¹³²Te, ¹³³Te, ^{133m}Te, ¹³⁴Te, ¹¹⁸I, ¹¹⁹I, ¹²⁰I, ^{120m}I, ¹²¹I, ¹²³I, ¹²⁴I, ¹²⁶I, ¹²⁸I, ¹³⁰I, ¹³¹I, ¹³²I, ^{132m}I, ¹³³I, ¹³⁴I, ¹³⁵I, ¹²⁰Xe, ¹²¹Xe, ¹²²Xe, ¹²³Xe, ¹²⁵Xe, ¹²⁷Xe, ¹³³Xe, ^{133m}Xe, ¹³⁵Xe, ^{135m}Xe, ¹³⁸Xe, ¹²⁵Cs, ¹²⁷Cs, ¹²⁹Cs, ¹³⁰Cs, ¹³¹Cs, ¹³²Cs, ¹³⁴Cs, ¹³⁵Cs, ¹³⁶Cs, ¹³⁸Cs, ¹²⁴Ba, ¹²⁶Ba, ¹²⁷Ba, ¹²⁸Ba, ¹²⁹Ba, ^{129m}Ba, ¹³¹Ba, ^{131m}Ba, ¹³³Ba, ¹³⁵Ba, ¹³⁹Ba, ¹⁴⁰Ba, ¹⁴¹Ba, ¹⁴²Ba, ¹²⁹La, ¹³¹La, ¹³²La, ¹³³La, ¹³⁵La, ¹⁴⁰La, ¹⁴¹La, ¹⁴²La, ¹⁴³La, ¹³⁰Ce, ¹³²Ce, ¹³³Ce, ^{133m}Ce, ¹³⁴Ce, ¹³⁵Ce, ¹³⁷Ce, ^{137m}Ce, ¹⁴¹Ce, ¹⁴³Ce, ¹⁴⁶Ce, ¹³⁴Pr, ^{134m}Pr, ¹³⁶Pr, ¹³⁷Pr, ^{138m}Pr, ¹³⁹Pr, ¹⁴²Pr, ¹⁴³Pr, ¹⁴⁴Pr, ¹⁴⁵Pr, ¹⁴⁶Pr, ¹⁴⁷Pr, ¹³⁵Nd, ¹³⁶Nd, ¹³⁷Nd, ¹³⁸Nd, ¹³⁹Nd, ^{139m}Nd, ¹⁴⁰Nd, ¹⁴¹Nd, ¹⁴⁷Nd, ¹⁴⁹Nd, ¹⁵¹Nd, ¹⁵²Nd, ¹⁴¹Pm, ¹⁴⁸Pm, ^{148m}Pm, ¹⁴⁹Pm, ¹⁵⁰Pm, ¹⁵¹Pm, ¹⁴⁰Sm, ¹⁴¹Sm, ^{141m}Sm, ¹⁴²Sm, ¹⁵³Sm, ¹⁵⁵Sm, ¹⁵⁶Sm, ¹⁴⁵Eu, ¹⁴⁶Eu, ¹⁴⁷Eu, ¹⁵⁰Eu, ^{152m}Eu, ¹⁵⁴Eu, ¹⁵⁶Eu, ¹⁵⁷Eu, ¹⁵⁸Eu, ¹⁵⁹Eu, ¹⁴⁵Gd, ¹⁴⁶Gd, ¹⁴⁷Gd, ¹⁴⁹Gd, ¹⁵⁹Gd, ¹⁴⁷Tb, ¹⁴⁸Tb, ¹⁴⁹Tb, ¹⁵⁰Tb, ¹⁵¹Tb, ¹⁵²Tb, ¹⁵³Tb, ¹⁵⁴Tb, ^{154m}Tb, ¹⁵⁵Tb, ¹⁵⁶Tb, ^{156m}Tb, ¹⁶¹Tb, ¹⁶³Tb, ¹⁵¹Dy, ¹⁵²Dy, ¹⁵³Dy, ¹⁵⁵Dy, ¹⁵⁷Dy, ¹⁶⁵Dy, ¹⁶⁶Dy, ¹⁵⁴Ho, ¹⁵⁵Ho, ¹⁵⁶Ho, ¹⁵⁷Ho, ^{158m}Ho, ¹⁵⁹Ho, ¹⁶¹Ho, ¹⁶²Ho, ^{162m}Ho, ¹⁶⁴Ho, ^{164m}Ho, ¹⁶⁶Ho, ¹⁶⁷Ho, ¹⁵⁶Er, ¹⁵⁷Er, ¹⁵⁸Er, ¹⁵⁹Er, ¹⁶⁰Er, ¹⁶¹Er, ¹⁶³Er, ¹⁶⁵Er, ¹⁶⁹Er, ¹⁷¹Er, ¹⁷²Er, ¹⁶¹Tm, ¹⁶²Tm, ¹⁶³Tm, ¹⁶⁵Tm, ¹⁶⁶Tm, ¹⁶⁷Tm, ¹⁷²Tm, ¹⁷³Tm, ¹⁷⁵Tm, ¹⁶²Yb, ¹⁶³Yb, ¹⁶⁴Yb, ¹⁶⁶Yb, ¹⁶⁷Yb, ¹⁶⁹Yb, ¹⁷⁵Yb, ¹⁷⁷Yb, ¹⁷⁸Yb, ¹⁶⁷Lu, ¹⁶⁹Lu, ¹⁷⁰Lu, ¹⁷¹Lu, ¹⁷²Lu, ^{176m}Lu, ¹⁷⁷Lu, ¹⁷⁸Lu, ^{178m}Lu, ¹⁷⁹Lu, ¹⁶⁸Hf, ¹⁷⁰Hf, ¹⁷³Hf, ^{177m}Hf, ^{179m}Hf, ^{180m}Hf, ¹⁸¹Hf, ^{182m}Hf, ¹⁸³Hf, ¹⁸⁴Hf, ¹⁷²Ta, ¹⁷³Ta, ¹⁷⁴Ta, ¹⁷⁵Ta, ¹⁷⁶Ta, ¹⁷⁷Ta, ¹⁷⁸Ta, ¹⁸⁰Ta, ^{182m}Ta, ¹⁸³Ta, ¹⁸⁴Ta, ¹⁸⁵Ta, ¹⁸⁶Ta, ¹⁷⁴W, ¹⁷⁵W, ¹⁷⁷W, ¹⁷⁸W, ¹⁷⁹W, ¹⁸⁷W, ¹⁹⁰W, ¹⁷⁷Re, ¹⁷⁸Re, ¹⁷⁹Re, ¹⁸¹Re, ¹⁸²Re, ^{182m}Re, ¹⁸⁴Re, ¹⁸⁶Re, ¹⁸⁸Re, ^{188m}Re, ¹⁸⁹Re, ^{190m}Re, ¹⁸⁰Os, ¹⁸¹Os, ¹⁸²Os, ¹⁸³Os, ^{183m}Os, ¹⁹¹Os, ¹⁹³Os, ¹⁹⁶Os, ¹⁸²Ir, ¹⁸³Ir, ¹⁸⁴Ir, ¹⁸⁵Ir, ¹⁸⁶Ir, ^{186m}Ir, ¹⁸⁷Ir, ¹⁸⁸Ir, ¹⁸⁹Ir, ¹⁹⁰Ir, ¹⁹⁴Ir, ¹⁹⁵Ir, ^{195m}Ir, ^{196m}Ir, ¹⁸⁴Pt, ¹⁸⁶Pt, ¹⁸⁷Pt, ¹⁸⁸Pt, ¹⁸⁹Pt, ¹⁹¹Pt, ¹⁹⁵Pt, ¹⁹⁷Pt, ^{197m}Pt, ¹⁹⁹Pt, ²⁰⁰Pt, ²⁰²Pt, ¹⁸⁶Au, ¹⁹⁰Au, ¹⁹¹Au, ¹⁹²Au, ¹⁹³Au, ¹⁹⁴Au, ¹⁹⁶Au, ^{196m}Au, ¹⁹⁸Au, ^{198m}Au, ¹⁹⁹Au, ²⁰⁰Au, ^{200m}Au, ¹⁹⁰Hg, ¹⁹¹Hg, ¹⁹²Hg, ¹⁹³Hg, ¹⁹⁵Hg, ^{195m}Hg, ¹⁹⁷Hg, ^{197m}Hg, ¹⁹⁹Hg, ²⁰³Hg, ¹⁹⁴Tl, ^{194m}Tl, ¹⁹⁵Tl, ¹⁹⁶Tl, ^{196m}Tl, ¹⁹⁷Tl, ¹⁹⁸Tl, ^{198m}Tl, ¹⁹⁹Tl, ²⁰⁰Tl, ²⁰¹Tl, ²⁰²Tl, ¹⁹⁴Pb, ¹⁹⁵Pb, ¹⁹⁶Pb, ^{197m}Pb, ¹⁹⁸Pb, ¹⁹⁹Pb, ^{199m}Pb, ²⁰⁰Pb, ²⁰¹Pb, ^{202m}Pb, ²⁰³Pb, ²⁰⁴Pb, ²⁰⁹Pb, ²¹¹Pb, ²¹²Pb, ²¹⁴Pb, ²⁰⁰Bi, ^{200m}Bi, ²⁰¹Bi, ²⁰²Bi, ²⁰³Bi, ²⁰⁴Bi, ²⁰⁵Bi, ²⁰⁶Bi, ²¹⁰Bi, ²¹²Bi, ^{212m}Bi, ²¹³Bi, ²¹⁴Bi, ²⁰⁰Po, ²⁰¹Po, ²⁰²Po, ²⁰³Po, ²⁰⁴Po, ²⁰⁵Po, ²⁰⁶Po, ²⁰⁷Po, ²⁰⁵At, ²⁰⁶At, ²⁰⁷At, ²⁰⁸At, ²⁰⁹At, ²¹⁰At, ²¹¹At, ²⁰⁸Rn, ²⁰⁹Rn, ²¹⁰Rn, ²¹¹Rn, ²¹²Rn, ²²¹Rn, ²²²Rn, ²²³Rn, ²¹²Fr, ²²²Fr, ²²³Fr, ²²³Ra, ²²⁴Ra, ²²⁵Ra, ²²⁷Ra, ²³⁰Ra, ²²⁴Ac, ²²⁵Ac, ²²⁶Ac, ²²⁸Ac, ²²⁹Ac, ²²⁶Th, ²²⁷Th, ²³¹Th, ²³³Th, ²³⁴Th, ²³⁶Th, ²²⁷Pa, ²²⁸Pa, ²²⁹Pa, ²³⁰Pa, ²³²Pa, ²³³Pa, ²³⁴Pa, ²³⁵Pa, ²²⁹U, ²³⁰U, ²³¹U, ²³⁷U, ²³⁹U, ²⁴⁰U, ²⁴²U, ²³¹Np, ²³²Np, ²³³Np, ²³⁴Np, ^{236m}Np, ²³⁸Np, ²³⁹Np, ²⁴⁰Np, ²⁴¹Np, ²³²Pu, ²³⁵Pu, ²³⁷Pu, ²⁴³Pu, ²⁴⁵Pu, ²⁴⁶Pu, ²³⁵Am, ²³⁷Am, ²³⁸Am, ²³⁹Am, ²⁴⁰Am, ²⁴²Am, ²⁴⁴Am, ^{244m}Am, ²⁴⁵Am, ²⁴⁶Am, ^{246m}Am, ²⁴⁷Am, ²³⁹Cm, ²⁴⁰Cm, ²⁴¹Cm, ²⁵¹Cm, ²⁴⁵Bk, ²⁴⁶Bk, ²⁴⁸Bk, ²⁵⁰Bk, ²⁵¹Bk, ²⁴⁴Cf, ²⁴⁵Cf, ²⁴⁶Cf, ²⁴⁷Cf, ²⁵³Cf, ²⁵⁵Cf, ²⁴⁹Es, ²⁵⁰Es, ^{250m}Es, ²⁵¹Es, ²⁵³Es, ^{254m}Es, ²⁵⁵Es, ^{256m}Es, ²⁵⁰Fm, ²⁵¹Fm, ²⁵²Fm, ²⁵⁴Fm, ²⁵⁵Fm, ²⁵⁵Md, ²⁵⁶Md, ²⁵⁷Md, ²⁵⁹No. Their properties are described in more detail, for instance, in Nuclear Data Sheets (Elsevier, Amsterdam, NL).

In an embodiment of the present disclosure, the radionuclide is used for diagnosis. In some embodiments, the radioactive isotope is selected from the group including, but not limited to, ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I. In some embodiments, the radionuclide is selected from ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, and ²⁰³Pb. In some embodiments, the radionuclide is selected from ¹⁸F, ⁶⁴Cu, ⁶⁸Ga, and ¹¹¹In. In an embodiment of the present disclosure, the radionuclide is ¹⁸F, whereby ¹⁸F forms a covalent bond to aluminium and aluminium forms a metal complex with the chelator. Methods and compositions for ¹⁸F labeling of proteins, peptides and other molecules are, for example, disclosed in WO 2012/082618. It will, however, also be acknowledged by a person skilled in the art that the use of said radionuclide is not limited to diagnostic purposes, but encompasses their use in therapy and theragnostics when conjugated to the compound of the disclosure.

In an embodiment of the present disclosure, the radionuclide is used for therapy. In some embodiments, the radioactive isotope is selected from ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, and ²¹¹At. In some embodiments, the radioactive isotope is selected from ⁴⁷Sc, ⁶⁷Cu, ⁹⁰Y, ¹⁶¹Tb, ¹⁷⁷Lu, ¹⁸⁸Re, ²¹²Pb, ²¹²Bi, ²¹³Bi, ²²⁵Ac, and ²²⁷Th. In some embodiments, the radionuclide is selected from ⁹⁰Y, ¹⁶¹Tb, ¹⁷⁷Lu, ²¹²Pb, ²²⁵Ac, and ²²⁷Th. It will, however, also be acknowledged by a person skilled in the art that the use of said radionuclide is not limited to therapeutic purposes, but encompasses their use in diagnostic and theragnostics when conjugated to the compound of the disclosure.

In an embodiment, the compound of the invention and disclosure, respectively, comprise a nuclide which is bound, preferably coordinatively bound, by a chelator forming part of the compound. It will be acknowledged and appreciated by a person skilled in the art that the coordination geometry of such complex of the nuclide and the chelator may vary. Embodiments of the structure, coordination geometry and overall complex charge for various chelate complexes are shown in Table 8.

TABLE 8
Selected chelate complexes with structure, coordination geometry and overall
complex charge (coordinate bonds between metal center and ligand are shown as dotted
lines).

| | | | Donor | | |
|---|---|---|---|---|---|
| | | | sphere | Coordination | |
| | | | (total | geometry | |
| | | | coordination | (nuclide- | Complex |
| Nuclide | Chelator | Structure | number) | chelator) | charge |
| Al(III)- F | NOTA | 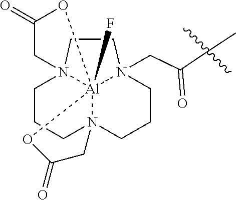 | N₃O₂ (6) | distorted octahedral^{b} | neutral |
| Cu(II) | DOTA | 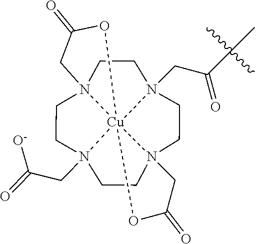 | N₄O₂ (6) | distorted octahedral^{a} | mono- anionic (−1) |
| | NODAGA | 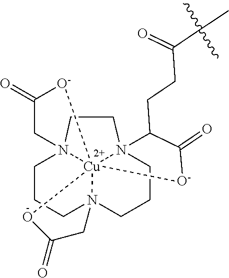 | N₃O₃ (6) | distorted trigonal prismatic^{a} | mono- anionic (−1) |
| Ga(III) | DOTA | 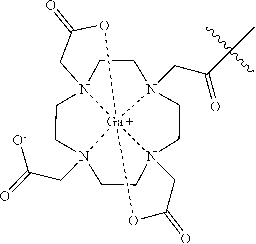 | N₄O₂ (6) | distorted octahedral^{a} | neutral |
| | NODAGA | 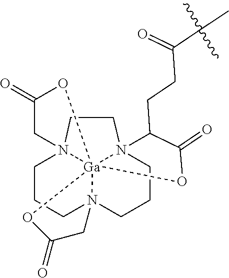 | N₃O₃ (6) | distorted octahedral^{a} | neutral |
| Y(III) | DOTA | 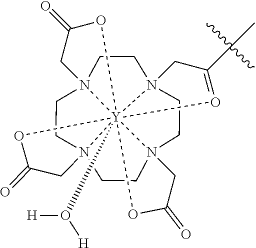 | N₄O₄ (9)^{c} | monocapped square antiprismatic^{a} | neutral |
| In(III) | DOTA | 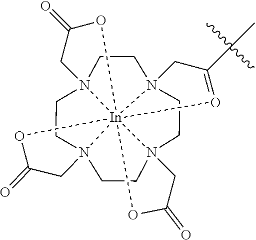 | N₄O₄ (8) | twisted square antiprismatic^{f} | neutral |
| | LSC (alternative PSC) | 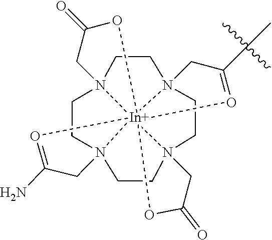 | Proposed; N₄O₄ (undetermined) | undetermined | mono- cationic (+1) |
| Tb(III) | DOTA | 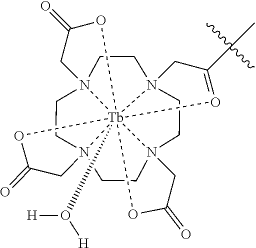 | N₄O₄ (9)^{c} | monocapped square antiprismatic^{d} | neutral |
| Lu(III) | DOTA | 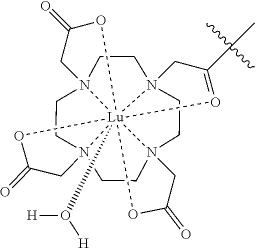 | N₄O₄ (9)^{c} | monocapped square antiprismatic^{b} | neutral |
| Pb(II) | DOTA | 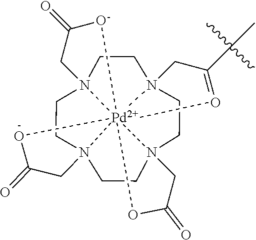 | N₄O₄ (8) | twisted square prismatic^{e} | mono- anionic (−1) |
| | DOTAM | 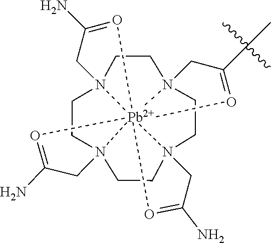 | N₄O₄ (8/9)^{g} | twisted square antiprismatic^{e} | di- cationic (+2) |
| | LSC (alternative PSC) | 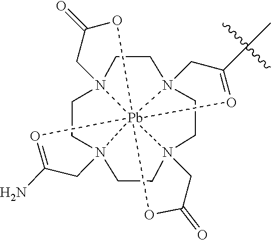 | Proposed: N₄O₄ undetermined | undetermined | neutral |
| Ac(III) | DOTA | 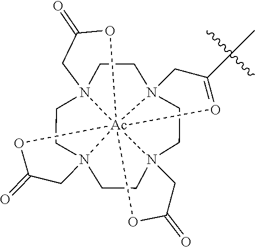 | Proposed: N₄O₄ undetermined | Proposed: capped inverted square antiprismatic^{e} | neutral |
| | Macropa | 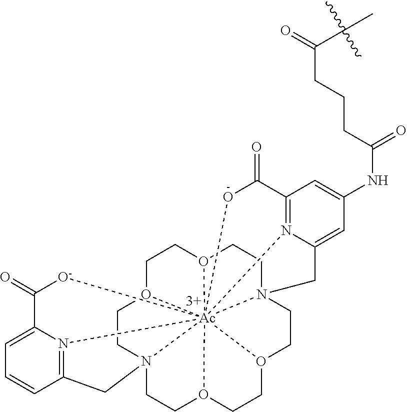 | Proposed: N₄O₆ undetermined | Proposed: irregular tridecahedron^{e} | mono- cationic (+1) |

^{a}Wadas et al., Chem. Rev. 2010, 100, 2858
^{b}Aime et al., Inorg. Chim. Acta 1996, 246, 423
^{c}The ligand DOTA acts as an octadentate ligand. To saturate the coordination sphere of the metal center, an additional (monodentate) ligand occupies the ninth coordination side. In aqueous solution, the additional ligand is usually a water molecule but can vary depending on the chemical composition of the environment. Exemplarily, the adjoining structure shows a water molecule as additional ligand.
^{d}Shiells et al., Dalton Trans. 2011, 40, 11451
^{e}Grieve et al., Aust. J. Chem. 2022, 75, 65
^{f}Liu et al., Inorg. Chem. 2003, 42, 8831
^{g}Two crystal structures of the Pb²⁺ complex of DOTAM have been published. The structures both showed the eight donor atoms encapsulating the ion but differed by the presence or absence of a water molecule weakly interacting with the Pb²⁺ ion (Grieve et al., Aust. J. Chem. 2022, 75, 65).
^{h}Archibald et al., EJNMMI radiopharm. chem. 2021, 6:30

In an embodiment, the compound of the disclosure is present as a pharmaceutically acceptable salt.

In certain embodiments, a “pharmaceutically acceptable salt” of a compound of the present disclosure is an acid salt or a base salt that is generally considered in the art to be suitable for use in contact with the tissues of human beings or animals without excessive toxicity or carcinogenicity, and, for example, without irritation, allergic response, or other problem or complication. Such salts include mineral and organic acid salts of basic residues, such as amines, as well as alkali or organic salts of acidic residues such as carboxylic acids. Compounds of the disclosure are capable of forming internal salts, which are also pharmaceutically acceptable salts.

Suitable pharmaceutically acceptable salts include, but are not limited to, salts of acids, such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to 4, i.e., 0, 1, 2, 3, or 4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of non-aqueous media, such as ether, ethyl acetate, ethanol, isopropanol or acetonitrile, is preferred.

In an embodiment, the compound of the disclosure is present as a pharmaceutically acceptable solvate.

In certain embodiments, a “pharmaceutically acceptable solvate” of a compound of the disclosure is a solvate of the compound of the disclosure formed by association of one or more solvent molecules to one or more molecules of a compound of the disclosure. In some embodiments, the solvent is one which is generally considered in the art to be suitable for use in contact with the tissues of human beings or animals without excessive toxicity or carcinogenicity, and for example, without irritation, allergic response, or other problem or complication. Such solvent includes an organic solvent, such as alcohols, ethers, esters and amines.

In an embodiment, the compound of the disclosure is present as a hydrate, preferably a pharmaceutically acceptable hydrate.

In certain embodiments, a “hydrate” of a compound of the disclosure is formed by association of one or more water molecules to one or more molecules of a compound of the disclosure. Such hydrates include, but are not limited to, a hemi-hydrate, mono-hydrate, dihydrate, trihydrate and tetrahydrate. Independent of the hydrate composition, all hydrates are generally considered as pharmaceutically acceptable.

The compound of the disclosure has a high binding affinity to PSMA. Because of this high binding affinity, the compound of the disclosure is effective as, useful as, and/or suitable as a targeting agent, where the target is PSMA and/or a cell and/or tissue expressing PSMA. In terms of cells and tissues thus targeted by the compound of the disclosure any cell and tissue, respectively, expressing PSMA is or may be targeted.

It is within the present disclosure that the compound of the disclosure is used or is for use in a method for the treatment of a disease as disclosed herein. In certain embodiments, such a method for the treatment of a disease as disclosed herein comprises the step of administering to a subject in need thereof a therapeutically effective amount of the compound of the disclosure. Such a method includes, but is not limited to, curative or adjuvant cancer treatment. It is used as palliative treatment where cure is not possible and the aim is for local disease control or symptomatic relief or as therapeutic treatment where the therapy has survival benefit and it can be curative.

The method for the treatment of a disease as disclosed herein includes the treatment of the diseases disclosed herein, including tumors and cancer, and may be used either as the primary therapy or as second, third, fourth, or last line therapy. It is also within the present disclosure to combine the compound of the disclosure with further therapeutic approaches. It is well known to the person skilled in the art that the precise treatment intent including curative, adjuvant, neoadjuvant, therapeutic, or palliative treatment intent will depend on the tumor type, location, and stage, as well as the general health of the patient.

In an embodiment of the present disclosure, the disease is selected from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, carcinoma, squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), prostate cancer (e.g., metastatic castration resistant prostate cancer), renal cancer (e.g., clear cell carcinoma), head cancer, neck cancer, head and neck cancer, lung cancer (e.g., non-small cell lung cancer), salivary gland cancer, breast cancer, colorectal cancer, esophageal cancer, stomach cancer, liver cancer (e.g., hepatocellular cancer), thyroid cancer, glioblastoma, glioma, gall bladder cancer, laryngeal cancer, leukemia/lymphoma, uterine cancer, skin cancer (e.g., melanoma), endocrine cancer, sarcoma, urinary cancer, pancreatic cancer, gastrointestinal cancer, ovarian cancer, cervical cancer, endometrial cancer, fallopian tube cancer, primary peritoneal cancer, hematological cancer (e.g., diffuse large B cell lymphoma, Hodgkin's lymphoma, Non-Hodgkin's lymphoma, follicular lymphoma, acute myeloid leukemia, or multiple myeloma), cancer of unknown primary, adenomas, and tumor neovasculature.

In some embodiments, the subjects treated with the presently disclosed compounds may be treated in combination with other non-surgical anti-proliferative (e.g., anti-cancer) drug therapy. In some embodiments, the compounds may be administered in combination with an anti-cancer compound such as a cytostatic compound. A cytostatic compound is a compound (e.g., a small molecule, a nucleic acid, or a protein) that suppresses cell growth and/or proliferation. In some embodiments, the cytostatic compound is directed towards the malignant cells of a tumor. In some embodiments, the cytostatic compound is one which inhibits the growth and/or proliferation of vascular smooth muscle cells or fibroblasts.

In some embodiments, the herein-described compounds are used or are for use in combination with a chemotherapeutic agent, e.g., a DNA damaging chemotherapeutic agent. Non-limiting examples of DNA damaging chemotherapeutic agents include topoisomerase I inhibitors, topoisomerase II inhibitors; alkylating agents; DNA intercalators; DNA intercalators and free radical generators such as bleomycin; and nucleoside mimetics.

In some embodiments, a compound described herein can be administered alone or in combination with one or more additional therapeutic agents. For example, the combination therapy can include a composition comprising a conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the additional therapeutic agent can be selected from cell cycle inhibitors, CDK inhibitors, radiation sensitizers, agents that upregulate PSMA expression, anti-angiogenesis agents, other drugs to reduce hypoxia to increase radiosensitivity, and/or kidney protectants. In some embodiments, the conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

Suitable anti-proliferative drugs or cytostatic compounds to be used in combination with the presently disclosed compounds include anti-cancer drugs. Numerous anti-cancer drugs which may be used are well known and include, but are not limited to: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azaribine; Azetepa; Azotomycin; Batimastat; Benzodepa; Bicalutamide; Bisantrene Hydrochloride; Bisnafide Dimesylate; Bizelesin; Bleomycin; Bleomycin Sulfate; Brequinar Sodium; Bropirimine; Bryostatin-1; Busulfan; Cactinomycin; Calusterone; Caracemide; Carbetimer; Carboplatin; Carmustine; Carubicin Hydrochloride; Carzelesin; Cedefingol; Celebrex; Chlorambucil; Cirolemycin; Cisplatin; Cladribine; Crisnatol Mesylate; Cyclophosphamide; Cytarabine; Dacarbazine; Dactinomycin; Daunorubicin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Doxorubicin Glucuronide; Cyano-morpholino Doxorubicin; 2-Pyrrolinodoxorubicin (2P-DOX), Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Epirubicin Glucuronide; Erbulozole; Esorubicin Hydrochloride; Estramustine; Estramustine Phosphate Sodium; Etanidazole; Etoposide; Etoposide Phosphate; Etoposide Glucuronide; Etoprine; Fadrozole Hydrochloride; Fazarabine; Fenretinide; Floxuridine (FUdR); 3′,5′-O-dioleoyl-FudR (FUdR-dO); Fludarabine; Fludarabine Phosphate; Fluorouracil; Fluorocitabine; Flutamide; Fluoxymesterone; Fosquidone; Fostriecin Sodium; Gemcitabine; Gemcitabine Hydrochloride; Hydroxyurea; Hydroxyprogesterone caproate; Idarubicin; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; L-asparaginase; Lanreotide Acetate; Letrozole; Leucovorin; Leuprolide Acetate; Liarozole Hydrochloride; Lometrexol Sodium; Lomustine; Losoxantrone Hydrochloride; Masoprocol; Maytansine; Mechlorethamine; Mechlorethamine Hydrochloride; Medroprogesterone acetate; Megestrol Acetate; Melengestrol Acetate; Melphalan; Menogaril; Mercaptopurine; 6-Mercaptopurine; Methotrexate; Methotrexate Sodium; Metoprine; Meturedepa; Mithramycin; Mitindomide; Mitocarcin; Mitocromin; Mitogillin; Mitomalcin; Mitomycin; Mitosper; Mitotane; Mitoxantrone; Mitoxantrone Hydrochloride; Mycophenolic Acid; Niraparib; Nocodazole; Nogalamycin; Olaparib; Ormaplatin; Oxisuran; Paclitaxel; Pegaspargase; Peliomycin; Pentamustine; Peplomycin Sulfate; Perfosfamide; Phenyl Butyrate, Pipobroman; Piposulfan; Piroxantrone Hydrochloride; Plicamycin; Plomestane; Porfimer Sodium; Porfiromycin; Prednimustine; Prednisone; Procarbazine; Procarbazine Hydrochloride; PSI-341, Puromycin; Puromycin Hydrochloride; Pyrazofurin; Riboprine; Rogletimide; Rucaparib; Safingol; Safingol Hydrochloride; Semustine; Semustine Streptozocin; Simtrazene; Sparfosate Sodium; Sparsomycin; Spirogermanium Hydrochloride; Spiromustine; Spiroplatin; Streptonigrin; Streptozocin; Sulofenur; Talazoparib; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan; Topotecan Hydrochloride; Toremifene Citrate; Trestolone Acetate; Triciribine Phosphate; Trimetrexate; Trimetrexate Glucuronate; Tubulozole Hydrochloride; Uracil Mustard; Uredepa; Vapreotide; Velaparib; Velcade; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine; Vinorelbine Tartrate; Vinrosidine Sulfate; Vinzolidine Sulfate; Vorozole; Zeniplatin; Zinostatin; and Zorubicin Hydrochloride.

Other anti-cancer drugs include, but are not limited to: 20-epi-1,25 dihydroxyvitamin D3; 5-ethynyluracil; abiraterone; acylfulvene; adecypenol; adozelesin; ALL-TK antagonists; ambamustine; amidox; amifostine; aminolevulinic acid; amrubicin; anagrelide; andrographolide; angiogenesis inhibitors; antagonist D; antagonist G; antarelix; anti-dorsalizing morphogenetic protein-1; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bisaziridinylspermine; bisnafide; bistratene A; bortezomib; breflate; budotitane; buthionine sulfoximine; calicheamicin; calcipotriol; calphostin C; camptothecin derivatives; canarypox IL-2; capecitabine; carboxamide-amino-triazole; carboxyamidotriazole; CaRest M3; CARN 700; cartilage derived inhibitor; casein kinase inhibitors (ICOS); castanospermine; cecropin B; cetrorelix; chlorins; chloroquinoxaline sulfonamide; cicaprost; cis-porphyrin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; daunomycin glucuronide; daunorubicin; dehydrodidemnin B; diethylstilbestrol; deslorelin; dexamethasone; dexifosfamide; dexrazoxane; dexverapamil; didemnin B; didox; diethylnorspermine; azacytidine; dihydro-5-azacytidine; dihydrotaxol, 9-; dioxamycin; diphenyl spiromustine; docosanol; dolasetron; doxifluridine; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; ethinyl estradiol; etoposide phosphate; exemestane; filgrastim; finasteride; flavopiridol; flezelastine; fluasterone; fludarabine; fluorodaunorunicin hydrochloride; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti-cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; saintopin; SarCNU; sarcophytol A; sargramostim; Sdi 1 mimetics; senescence derived inhibitor 1; sense oligonucleotides; signal transduction inhibitors; signal transduction modulators; single chain antigen binding protein; sizofuran; SN-38; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen; tamoxifen methiodide; tauromustine; taxanes; tazarotene; tecogalan sodium; tegafur; tellurapyrylium; telomerase inhibitors; temozolomide; testosterone proprionate, tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; titanocene dichloride; topsentin; toremifene; totipotent stem cell factor; translation inhibitors; tretinoin; triacetyluridine; triciribine; tropisetron; turosteride; tyrosine kinase inhibitors; tyrphostins; UBC inhibitors; ubenimex; urogenital sinus-derived growth inhibitory factor; urokinase receptor antagonists; variolin B; vector system, erythrocyte gene therapy; velaresol; veramine; verdins; vinorelbine; vinxaltine; vitaxin; zanoterone; zilascorb; zinostatin stimalamer; abrin; ricine; ribonuclease; onconase; rapLR1; DNase I; Staphylococcal enterotoxin-A; pokeweed antiviral protein; gelonin; diphtheria toxin; Pseudomonas exotoxin; and Pseudomonas endotoxin; or combinations of these.

In some embodiments, the drug to be used in combination with the disclosed compounds is selected from duocarmycin and its analogues, dolastatins, combretastatin, calicheamicin, N-acetyl-γ-calicheamycin (CMC), a calicheamycin derivative, maytansine and analogues thereof, DM-I, auristatin E, auristatin EB (AEB), auristatin EFP (AEFP), monomethyl auristatin E (MMAE), monomethyl auristatin F (MMAF), tubulysin, disorazole, the epothilones, Paclitaxel, docetaxel, Topotecan, echinomycin, estramustine, cemadotine, eleutherobin, methopterin, actinomycin, daunorubicin, the daunorubicin conjugates, mitomycin C, mitomycin A, vincristine, retinoic acid, camptothecin, a camptothecin derivative, SN38, maytansine, a derivative of the maytansinoid type, DM1, DM4, TK1, amanitin, a pyrrolobenzodiazepine, a pyrrolobenzodiazepine dimer, methotrexate, ilomedine, aspirin, an IMIDs, lenalidomide, pomalidomide.

The presently disclosed compounds can also be used in combination with any of the following treatments:

Therapy in combination with compounds targeting the androgen receptor, including androgen depletion approaches and antiandrogens. Such inhibitors include but are not limited to enzalutamide, apalutamide, darolutamide, etc.

Therapy in combination with inhibitors of Poly(ADP-ribose) polymerases (PARP), a class of chemotherapeutic agents directed at targeting cancers with defective DNA-damage repair (Yuan, et al., Expert Opin Ther Pat, 2017, 27: 363). Such PARP inhibitors include but are not limited to olaparib, rucaparib, velaparib, niraparib, talazoparib, pamiparib, iniparib, E7449, and A-966492.

Therapy in combination with inhibitors of signaling pathways and mechanisms leading to repair of DNA single and double strand breaks as, e.g., nuclear factor-kappaB signaling (Pilie, et al., Nat Rev Clin Oncol, 2019, 16: 81; Zhang, et al., Chin J Cancer, 2012, 31: 359). Such inhibitors include but are not limited to inhibitors of ATM and ATR kinases, checkpoint kinase 1 and 2, DNA-dependent protein kinase, and WEE1 kinase (Pilie, et al., Nat Rev Clin Oncol, 2019, 16: 81).

Therapy in combination with an immunomodulator (Khalil, et al., Nat Rev Clin Oncol, 2016, 13: 394), a cancer vaccine (Hollingsworth, et al., NPJ Vaccines, 2019, 4: 7), an immune checkpoint inhibitor (e.g., PD-1, PD-L1, CTLA-4-inhibitor) (Wei, et al., Cancer Discov, 2018, 8: 1069), a Cyclin-D-Kinase 4/6 inhibitor (Goel, et al., Trends Cell Biol, 2018, 28: 911), an antibody being capable of binding to a tumor cell and/or metastases and being capable of inducing antibody-dependent cellular cytotoxicity (ADCC) (Kellner, et al., Transfus Med Hemother, 2017, 44: 327), a T cell- or NK cell engager (e.g., bispecific antibodies) (Yu, et al., J Cancer Res Clin Oncol, 2019, 145: 941), a cellular therapy using expanded autologous or allogeneic immune cells (e.g., chimeric antigen receptor T (CAR-T) cells) (Khalil, et al., Nat Rev Clin Oncol, 2016, 13: 394). Immune checkpoint inhibitors include, but are not limited to nivolumab, ipilimumab, pembrolizumab, atezolizumab, avelumab, durvalumab, and cemiplimab.

According to the present disclosure, the compounds may be administered prior to, concurrent with, or following other anti-cancer compounds. The administration schedule may involve administering the different agents in an alternating fashion. In other embodiments, the compounds may be delivered before and during, or during and after, or before and after, or before and during and after treatment with other therapies. In some embodiments, the compound is administered more than 24 hours before the administration of the other anti-proliferative treatment. In some embodiments, more than one anti-proliferative therapy may be administered to a subject. For example, the subject may receive the present compounds, in combination with both surgery and at least one other anti-proliferative compound. In some embodiments, the compound may be administered in combination with more than one anti-cancer drug.

In some embodiments, the compounds of the present disclosure are used to detect cells and tissues overexpressing PSMA, whereby such detection is achieved by conjugating a detectable label to the compounds of the disclosure, for example a detectable radionuclide, or by attaching a dye to the peptide. In some embodiments, the cells and tissues detected are diseased cells and tissues and/or are either a or the cause for the disease and/or the symptoms of the disease, or are part of the pathology underlying the disease. In some embodiments, the diseased cells and tissues are causing and/or are part of an oncology indication (e.g., neoplasms, tumors, and cancers).

In some embodiments, the compounds of the present disclosure are used to treat cells and tissues overexpressing prostate specific membrane antigen (PSMA). In some embodiments, the cells and tissues treated are diseased cells and tissues and/or are either a or the cause for the disease and/or the symptoms of the disease, or are part of the pathology underlying the disease. In some embodiments, the diseased cells and tissues are causing and/or are part of an oncology indication (e.g., neoplasms, tumors, and cancers) and the therapeutic activity is achieved by conjugating a therapeutically active nuclide to the compounds of the present disclosure, for example, a therapeutically active radionuclide.

In a further embodiment, the compounds of the present disclosure are administered in therapeutically effective amounts. In some embodiments, a therapeutically effective amount is a dosage of the compound sufficient to provide a therapeutically or medically desirable result or effect in the subject to which the compound is administered. The therapeutically effective amount will vary with the particular condition being treated, the age and physical condition of the subject being treated, the severity of the condition, the duration of the treatment, the nature of the concurrent or combination therapy (if any), the specific route of administration and other factors within the knowledge and expertise of a healthcare practitioner. For example, in connection with methods directed towards treating subjects having a condition characterized by abnormal cell proliferation, an effective amount to inhibit proliferation would be an amount sufficient to reduce or halt altogether the abnormal cell proliferation so as to slow or halt the development of or the progression of a cell mass, such as, for example, a tumor. In an embodiment, and as preferably used herein, the term “inhibit” embraces all of the foregoing.

In some embodiments, a therapeutically effective amount will be an amount necessary to extend the dormancy of micrometastases or to stabilize any residual primary tumor cells following surgical or drug therapy.

Generally, a therapeutically effective amount may vary based on factors, such as the subject's age, condition, and sex, as well as the nature and extent of the disease in the subject, all of which can be determined by one of ordinary skill in the art. The dosage may be adjusted by the individual physician or veterinarian, particularly in the event of any complication. In some embodiments, a therapeutically effective amount includes, but not is limited to, an amount in a range from 0.1 μg/kg to about 2000 mg/kg, or from 1.0 μg/kg to about 1000 mg/kg, or from about 0.1 mg/kg to about 500 mg/kg, or from about 1.0 mg/kg to about 100 mg/kg, in one or more dose administrations daily, for one or more days. If desired, the effective daily dose of the active compound may be administered as two, three, four, five, six, or more sub-doses, for example administered separately at appropriate intervals throughout the day, optionally, in unit dosage forms. In some embodiments, the compounds are administered for more than 7 days, more than 10 days, more than 14 days, or more than 20 days. In some embodiments, the compound is administered over a period of weeks or months or years. In some embodiments, the compound is delivered on alternate days. For example, the agent is delivered every two days, or every three days, or every four days, or every five days, or every six days, or every week, or every month.

In some embodiments, the compounds of the present disclosure are for use in the treatment and/or prevention of a disease, whereby such treatment is radionuclide therapy.

For example, radionuclide therapy makes use of or is based on different forms of radiation emitted by a radionuclide. Such radiation can, for example, be any one of radiation of photons, radiation of electrons including but not limited to β-particles and Auger-electrons, radiation of protons, radiation of neutrons, radiation of positrons, radiation of α-particles or an ion beam. Depending on the kind of particle or radiation emitted by said radionuclide, radionuclide therapy can, for example, be distinguished as photon radionuclide therapy, electron radionuclide therapy, proton radionuclide therapy, neutron radionuclide therapy, positron radionuclide therapy, α-particle radionuclide therapy or ion beam radionuclide therapy. All of these forms of radionuclide therapy are encompassed by the present disclosure, and all of these forms of radionuclide therapy can be realized by the compound of the disclosure, wherein a radionuclide attached to the compound of the disclosure, is providing for this kind of radiation.

Radionuclide therapy preferably works by damaging the DNA of cells. The damage is caused by a photon, electron, proton, neutron, positron, α-particle or ion beam directly or indirectly ionizing the atoms which make up the DNA chain. Indirect ionization happens as a result of the ionization of water, forming free radicals, notably hydroxyl radicals, which then damage the DNA.

In the most common forms of radionuclide therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly.

Oxygen is a potent radiosensitizer, increasing the effectiveness of a given dose of radiation by forming DNA-damaging free radicals. Therefore, use of high pressure oxygen tanks, blood substitutes that carry increased oxygen, hypoxic cell radiosensitizers such as misonidazole and metronidazole, and hypoxic cytotoxins, such as tirapazamine may be applied.

Other factors that are considered when selecting a radioactive dose include whether the patient is receiving chemotherapy, whether radiation therapy is being administered before or after surgery, and the degree of success of surgery.

The total radioactive dose may be fractionated, i.e., spread out over time in one or more treatments for one or more of several important reasons. For example, fractionation allows normal cells time to recover, while tumor cells are generally less efficient in repair between fractions. For example, fractionation also allows tumor cells that were in a relatively radioresistant phase of the cell cycle during one treatment to cycle into a sensitive phase of the cycle before the next fraction is given. Similarly, tumor cells that were chronically or acutely hypoxic and, therefore, more radioresistant, may reoxygenate between fractions, improving the tumor cell kill.

It is generally known that different cancers respond differently to radiation therapy. The response of a cancer to radiation is described by its radiosensitivity. Highly radiosensitive cancer cells are rapidly killed by modest doses of radiation. These include leukemias, most lymphomas, and germ cell tumors.

It is important to distinguish radiosensitivity of a particular tumor, which to some extent is a laboratory measure, from “curability” of a cancer by an internally delivered radioactive dose in actual clinical practice. For example, leukemias are not generally curable with radiotherapy, because they are disseminated through the body. Lymphoma may be radically curable if it is localized to one area of the body. Similarly, many of the common, moderately radioresponsive tumors can be treated with curative doses of radioactivity if they are at an early stage. This applies, for example, to non-melanoma skin cancer, head and neck cancer, non-small cell lung cancer, cervical cancer, anal cancer, and prostate cancer.

The response of a tumor to radiotherapy is also related to its size. For complex reasons, very large tumors do not respond as well to radiation as smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radionuclide therapy. A third technique is to enhance the radiosensitivity of the cancer by giving certain drugs during a course of radiotherapy. Examples of radiosensitizing drugs include, but are not limited to Cisplatin, Nimorazole, and Cetuximab.

Introperative radiotherapy is a special type of radiotherapy that is delivered immediately after surgical removal of the cancer. This method has been employed in breast cancer (TARGeted Introperative radioTherapy), brain tumors and rectal cancers.

Radionuclide therapy is in itself painless. Many low-dose palliative treatments cause minimal or no side effects. Treatment with higher doses may cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radionuclide, dose, fractionation, concurrent chemotherapy), and the patient.

It is within the present disclosure that the method for the treatment of a disease of the invention may realize each and any of the above strategies which are as such known in the art, and which insofar constitute further embodiments of the disclosure.

It is also within the present disclosure that the compound of the disclosure is used in a method for the diagnosis of a disease as disclosed herein. In some embodiments, such a method comprises the step of administering to a subject in need thereof a diagnostically effective amount of the compound of the disclosure.

In accordance with the present disclosure, an imaging method is selected from the group consisting of scintigraphy, Single Photon Emission Computed Tomography (SPECT), Positron Emission Tomography (PET), computed tomography, and combinations thereof.

Scintigraphy is a form of diagnostic test or method used in nuclear medicine, wherein radiopharmaceuticals are internalized by cells, tissues and/or organs, for example, internalized in vivo, and radiation emitted by said internalized radiopharmaceuticals is captured by external detectors (gamma cameras) to form and display two-dimensional images. In contrast thereto, SPECT and PET forms and displays three-dimensional images. Because of this, SPECT and PET are classified as separate techniques to scintigraphy, although they also use gamma cameras to detect internal radiation. Scintigraphy is unlike a diagnostic X-ray where external radiation is passed through the body to form an image.

Single Photon Emission Tomography (SPECT) scans are a type of nuclear imaging technique using gamma rays. They are very similar to conventional nuclear medicine planar imaging using a gamma camera. Before the SPECT scan, the patient is injected with a radiolabeled chemical emitting gamma rays that can be detected by the scanner. A computer collects the information from the gamma camera and translates this into two-dimensional cross-sections. These cross-sections can be added back together to form a three-dimensional image of an organ or a tissue. SPECT involves detection of gamma rays emitted singly, and sequentially, by the radionuclide provided by the radiolabeled chemical. To acquire SPECT images, the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. This gives a total scan time of 15-20 minutes. Multi-headed gamma cameras are faster. Since SPECT acquisition is very similar to planar gamma camera imaging, the same radiopharmaceuticals may be used.

Positron Emitting Tomography (PET) is a non-invasive, diagnostic imaging technique for measuring the biochemical status or metabolic activity of cells within the human body. PET is unique since it produces images of the body's basic biochemistry or functions. Traditional diagnostic techniques, such as X-rays, computed tomography scans, or MRI, produce images of the body's anatomy or structure. The premise with these techniques is that any changes in structure or anatomy associated with a disease can be seen. Biochemical processes are also altered by a disease, and may occur before any gross changes in anatomy. PET is an imaging technique that can visualize some of these early biochemical changes. PET scanners rely on radiation emitted from the patient to create the images. Each patient is given a minute amount of a radioactive pharmaceutical that either closely resembles a natural substance used by the body or binds specifically to a receptor or molecular structure. As the radioisotope undergoes positron emission decay (also known as positive beta decay), it emits a positron, the antiparticle counterpart of an electron. After traveling up to a few millimeters, the positron encounters an electron and annihilates, producing a pair of annihilation (gamma) photons moving in opposite directions. These are detected when they reach a scintillation material in the scanning device, creating a burst of light, which is detected by photomultiplier tubes or silicon avalanche photodiodes. The technique depends on simultaneous or coincident detection of the pair of photons. Photons that do not arrive in pairs, i.e., within a few nanoseconds, are ignored. All coincidences are forwarded to the image processing unit where the final image data is produced using image reconstruction procedures.

SPECT/computed tomography and PET/computed tomography are the combination of SPECT and PET with computed tomography. The key benefits of combining these modalities are improving the reader's confidence and accuracy. With traditional PET and SPECT, the limited number of photons emitted from the area of abnormality produces a very low-level background that makes it difficult to anatomically localize the area. Adding computed tomography helps determine the location of the abnormal area from an anatomic perspective and categorize the likelihood that this represents a disease.

It is within the present disclosure that the method for the diagnosis of a disease of the disclosure may realize each and any of the above strategies which are as such known in the art, and which insofar constitute further embodiments of the disclosure.

In some embodiments, compounds of the present disclosure can be useful to stratify patients, i.e., to create subsets within a patient population that provide more detailed information about how the patient will respond to a given drug. Stratification can be a critical component to transforming a clinical trial from a negative or neutral outcome to one with a positive outcome by identifying the subset of the population most likely to respond to a novel therapy.

Stratification includes the identification of a group of patients with shared “biological” characteristics to select the optimal management for the patients and achieve the best possible outcome in terms of risk assessment, risk prevention and achievement of the optimal treatment outcome.

In some embodiments, a compound of the present disclosure may be used to assess or detect, a specific disease as early as possible (which is a diagnostic use), the risk of developing a disease (which is a susceptibility/risk use), the evolution of a disease including indolent vs. aggressive (which is a prognostic use) and it may be used to predict the response and the toxicity to a given treatment (which is a predictive use).

It is also within the present disclosure that the compounds of the disclosure may be used in a theragnostic method. The concept of theragnostics is to combine a therapeutic agent with a corresponding diagnostic test that can increase the clinical use of the therapeutic drug. The concept of theragnostics is becoming increasingly attractive and is widely considered the key to improving the efficiency of drug treatment by helping doctors identify patients who might profit from a given therapy and hence avoid unnecessary treatments.

The concept of theragnostics is to combine a therapeutic agent with a diagnostic test that allows doctors to identify those patients who will benefit most from a given therapy. In an embodiment, a compound of the present disclosure is used for the diagnosis of a patient, i.e., identification and localization of the primary tumor mass as well as potential local and distant metastases. Furthermore, the tumor volume can be determined, especially utilizing three-dimensional diagnostic modalities such as SPECT or PET. Only those patients having PSMA-positive tumor masses and who, therefore, might profit from a given therapy are selected for a particular therapy and hence unnecessary treatments are avoided. For example, such therapy is a PSMA targeted therapy using a compound of the present disclosure. In some embodiments, chemically identical tumor-targeted diagnostics, including, for example, imaging diagnostics for scintigraphy, PET or SPECT and radiotherapeutics are applied. Such compounds only differ in the radionuclide and therefore usually have a very similar if not identical pharmacokinetic profile. This can be realized using a chelator and a diagnostic or therapeutic radiometal. Alternatively, this can be realized using a precursor for radiolabeling and radiolabeling with either a diagnostic or a therapeutic radionuclide. In one embodiment diagnostic imaging is used by means of quantification of the radiation of the diagnostic radionuclide and subsequent dosimetry which is known to those skilled in the art and the prediction of drug concentrations in the tumor compared to vulnerable side effect organs. Thus, a truly individualized drug dosing therapy for the patient is achieved.

In some embodiments, the theragnostic method is realized with only one theragnostically active compound such as a compound of the present disclosure labeled with a radionuclide emitting diagnostically detectable radiation (e.g., positrons or gamma rays) as well as therapeutically effective radiation (e.g., electrons or alpha particles).

The disclosure also contemplates a method of intraoperatively identifying/disclosing diseased tissues expressing PSMA in a subject. Such method uses a compound of the disclosure, whereby in some embodiments such compound of the disclosure comprises a diagnostically active agent such as a diagnostically active radionuclide.

According to a further embodiment of the disclosure, the compound of the disclosure, particularly if complexed with a radionuclide, may be employed as adjunct or adjuvant to any other tumor treatment including, surgery as the primary method of treatment of most isolated solid cancers, radiation therapy involving the use of ionizing radiation in an attempt to either cure or improve the symptoms of cancer using either sealed internal sources in the form of brachytherapy or external sources, chemotherapy such as alkylating agents, antimetabolites, anthracyclines, plant alkaloids, topoisomerase inhibitors, and other antitumor agents, hormone treatments that modulate tumor cell behavior without directly attacking those cells, targeted agents which directly target a molecular abnormality in certain types of cancer including monoclonal antibodies and tyrosine kinase inhibitors, angiogenesis inhibitors, immunotherapy, cancer vaccination, palliative care including actions to reduce the physical, emotional, spiritual, and psycho-social distress to improve the patient's quality of life and alternative treatments including a diverse group of health care systems, practices, and products that are not part of conventional medicine.

In an embodiment of the methods of the disclosure, the subject is a patient. In an embodiment, a patient is a subject which has been diagnosed as suffering from or which is suspected of suffering from or which is at risk of suffering from or developing a disease, whereby the disease is a disease as described herein, a disease involving prostate specific membrane antigen (PSMA).

Dosages employed in practicing the methods for treatment and diagnosis, respectively, where a radionuclide is used and more specifically attached to or part of the compound of the disclosure will vary depending, e.g., on the particular condition to be treated, for example the known radiosensitivity of the tumor type, the volume of the tumor and the therapy desired. In general, the dose is calculated on the basis of radioactivity distribution to each organ and on observed target uptake. A γ-emitting complex may be administered once or at several times for diagnostic imaging. In animals, an indicated dose range may be, for example, from 0.1 ng/kg to 5 mg/kg of the compound of the disclosure complexed, e.g., with 1 kBq to 200 MBq of a γ-emitting radionuclide, including, but not limited to, ¹¹¹In or ⁸⁹Zr. An α- or β-emitting complex of the compound of the disclosure may be administered at several time points, e.g., over a period of 1 to 3 weeks or longer. In animals, an indicated dosage range may be, for example, from 0.1 ng/kg to 5 mg/kg of the compound of the disclosure complexed, e.g., with 1 kBq to 200 MBq of an α- or β-emitting radionuclide, including, but not limited to, ²²⁵Ac or ¹⁷⁷Lu. In larger mammals, including, for example, humans, an indicated dosage range may be, for example, from 0.1 ng/kg to 5 mg/kg or for example 0.1 ng/kg to 100 μg/kg of the compound of the disclosure complexed with, e.g., 10 to 1000 MBq of ay-emitting radionuclide, including, but not limited to, ¹¹¹In or ⁸⁹Zr. In larger mammals, including, for example, humans, an indicated dosage range may be, for example, from 0.1 ng/kg to 5 mg/kg or for example, from 0.1 ng/kg to 100 μg/kg of the compound of the disclosure complexed with, e.g., 1 to 100000 MBq of an α- or β-emitting radionuclide, including, but not limited to, ²²⁵Ac or ¹⁷⁷Lu.

In certain embodiments, uptake can be measured in terms of absorbed dose (mGy/MBq), SUVmax, and/or SUVmean. In animals, uptake across tissues is reported in percent injected dose/gram (% ID/g). Sensitivity to radiation is tumor and non-tumor tissue dependent. The favorable tumor to non-tumor tissue uptake of the present compounds allows delivery of a radioactive nuclide at a dose that could reduce tumor growth, or partially or completely destroys the tumor. At such dose, no permanent or critical damage to non-tumor tissue is expected.

In a further aspect, the instant disclosure is related to a composition and a pharmaceutical composition in particular, comprising the compound of the disclosure.

The pharmaceutical composition of the present disclosure comprises at least one compound of the disclosure and, optionally, one or more carrier substances, excipients and/or adjuvants. The pharmaceutical composition may additionally comprise, for example, one or more of water, buffers such as, e.g., neutral buffered saline or phosphate buffered saline, ethanol, mineral oil, vegetable oil, dimethylsulfoxide, carbohydrates such as e.g., glucose, mannose, sucrose or dextrans, mannitol, proteins, adjuvants, polypeptides or amino acids such as glycine, antioxidants, chelating agents such as EDTA or glutathione and/or preservatives. Furthermore, one or more other active ingredients may, but need not, be included in the pharmaceutical composition of the disclosure.

The pharmaceutical composition of the disclosure may be formulated for any appropriate route of administration, including, for example, topical such as, e.g., transdermal or ocular, oral, buccal, nasal, vaginal, rectal or parenteral administration. In an embodiment, and as preferably used herein, the term parenteral includes subcutaneous, intradermal, intravascular such as, e.g., intravenous, intramuscular, intrathecal and intraperitoneal injection, as well as any similar injection or infusion technique. In some embodiments, the route of administration is intravenous administration.

In an embodiment of the disclosure the compound of the disclosure comprising a radionuclide is administered by any conventional route, in particular intravenously, e.g., in the form of injectable solutions or suspensions. The compound of the disclosure may also be administered advantageously by infusion, e.g., by an infusion of 30 to 60 min.

In some embodiments, depending on the site of the tumor, the compound of the disclosure may be administered as close as possible to the tumor site, e.g., by means of a catheter. Such administration may be carried out directly into the tumor tissue or into the surrounding tissue or into the afferent blood vessels. The compound of the disclosure may also be administered repeatedly in doses, including, in some embodiments, in divided doses.

According to an embodiment of the disclosure, a pharmaceutical composition of the disclosure comprises a stabilizer, e.g., a free radical scavenger, which inhibits autoradiolysis of the compound of the disclosure. Suitable stabilizers include, e.g., serum albumin, ascorbic acid, retinol, gentisic acid or a derivative thereof, or an amino acid infusion solution such, e.g., used for parenteral protein feeding, for example, free from electrolyte and glucose, for example a commercially available amino acid infusion such as Proteinsteril® KE Nephro. In some embodiments, ascorbic acid and gentisic acid are used.

A pharmaceutical composition of the disclosure may comprise further additives, e.g., an agent to adjust the pH between 7.2 and 7.4, e.g., sodium or ammonium acetate or Na₂HPO₄. In some embodiments, the stabilizer is added to the non-radioactive compound of the disclosure and introduction of the radionuclide, for instance the complexation with the radionuclide, is performed in the presence of the stabilizer, either at room temperature or, for example, at a temperature of from 40 to 120° C. The complexation may conveniently be performed under air free conditions, e.g., under N₂ or Ar. In some embodiments, further stabilizer may be added to the composition after complexation.

Excretion of the compound of the disclosure, particularly if the compound comprises a radionuclide, essentially takes place through the kidneys. In some embodiments, further protection of the kidneys from radioactivity accumulation may be achieved by administration of lysine or arginine or an amino acid solution having a high content of lysine and/or arginine, e.g., a commercially available amino acid solution such as Synthamin®-14 or -10, prior to the injection of or together with the compound of the disclosure, particularly if the compound comprises a radionuclide. In some embodiments, protection of the kidneys may also be achieved by administration of plasma expanders, such as, e.g., gelofusine, either instead of or in addition to amino acid infusion. In some embodiments, protection of the kidneys may also be achieved by administration of diuretics providing a means of forced diuresis which elevates the rate of urination. Such diuretics include high ceiling loop diuretics, thiazides, carbonic anhydrase inhibitors, potassium-sparing diuretics, calcium-sparing diuretics, osmotic diuretics and low ceiling diuretics. In some embodiments, a pharmaceutical composition of the disclosure may contain, apart from a compound of the disclosure, at least one of these further compounds intended for or suitable for kidney protection, including, for example, kidney protection of the subject to which the compound of the disclosure is administered.

It will be understood by a person skilled in the art that the compounds of the disclosure are disclosed herein for use in various methods. It will be further understood by a person skilled in the art that the composition of the disclosure and the pharmaceutical composition of the disclosure can be equally used in said various methods. It will also be understood by a person skilled in the art that the composition of the disclosure and the pharmaceutical composition are disclosed herein for use in various methods. It will be equally understood by a person skilled in the art that the compounds of the disclosure can be equally used in said various methods.

It will be acknowledged by a person skilled in the art that the composition and/or the pharmaceutical composition as disclosed herein may contain one or more further compounds in addition to the compound of the disclosure. To the extent that such one or more further compounds are disclosed herein as being part of the composition of the disclosure and/or of the pharmaceutical composition of the disclosure, it will be understood that such one or more further compounds can be administered separately from the compound of the disclosure to the subject which is exposed to or the subject of a method of the disclosure. Such administration of the one or more further compounds can be performed prior to, concurrently with or after the administration of the compound of the invention. It will also be acknowledged by a person skilled in the art that in a method of the invention, apart from a compound of the invention, one or more further compounds may be administered to a subject. Such administration of the one or more further compounds can be performed prior to, concurrently with or after the administration of the compound of the disclosure. To the extent that such one or more further compounds are disclosed herein as being administered as part of a method of the disclosure, it will be understood that such one or more further compounds are part of a composition of the disclosure and/or of a pharmaceutical composition of the disclosure. It is within the present disclosure that the compound of the disclosure and the one or more further compounds may be contained in the same or a different formulation. It is also within the present disclosure that the compound of the disclosure and the one or more further compounds are not contained in the same formulation, but are contained in the same package containing a first formulation comprising a compound of the disclosure, and a second formulation comprising the one or more further compounds, whereby the type of formulation may be the same or may be different.

It is within the present disclosure that more than one type of a compound of the disclosure may be contained in the composition of the disclosure and/or the pharmaceutical composition of the disclosure. It is also within the present disclosure that more than one type of a compound of the disclosure may be used, preferably administered, in a method of the disclosure.

It will be acknowledged that a composition of the disclosure and a pharmaceutical composition of the disclosure may be manufactured in conventional manner.

Radiopharmaceuticals have decreasing content of radioactivity with time, as a consequence of the radioactive decay. The physical half-life of the radionuclide is often short for radiopharmaceutical diagnostics. In these cases, the final preparation has to be done shortly before administration to the patient. This is in particular the case for positron emitting radiopharmaceuticals for tomography (PET radiopharmaceuticals). It often leads to the use of semi-manufactured products such as radionuclide generators, radioactive precursors and kits.

In some embodiments, a kit of the disclosure comprises apart from one or more than one compounds of the disclosure typically at least one of the followings: instructions for use, final preparation and/or quality control, one or more optional excipient(s), one or more optional reagents for the labeling procedure, optionally one or more radionuclide(s) with or without shielded containers, and optionally one or more device(s), whereby the device(s) is/are selected from the group comprising a labeling device, a purification device, an analytical device, a handling device, a radioprotection device or an administration device.

Shielded containers known as “pigs” for general handling and transport of radiopharmaceutical containers come in various configurations for holding radiopharmaceutical containers such as bottles, vials, syringes, etc. One form includes a removable cover that allows access to the held radiopharmaceutical container. When the pig cover is in place, the radiation exposure is acceptable.

In some embodiments, a labeling device is selected from the group of open reactors, closed reactors, microfluidic systems, nanoreactors, cartridges, pressure vessels, vials, temperature controllable reactors, mixing or shaking reactors and combinations thereof.

In some embodiments, a purification device is selected from the group of ion exchange chromatography columns or devices, size-exclusion chromatography columns or devices, affinity chromatography columns or devices, gas or liquid chromatography columns or devices, solid phase extraction columns or devices, filtering devices, centrifugations vials columns or devices and combinations thereof.

In some embodiments, an analytical device is selected from the group of tests or test devices to determine the identity, radiochemical purity, radionuclidic purity, content of radioactivity and specific radioactivity of the radiolabeled compound and combinations thereof.

In some embodiments, a handling device is selected from the group consisting of devices for mixing, diluting, dispensing, labeling, injecting and administering radiopharmaceuticals to a subject and combinations thereof.

In some embodiments, a radioprotection device is used in order to protect doctors and other personnel from radiation when using therapeutic or diagnostic radionuclides. In some embodiments, the radioprotection device is selected from the group consisting of devices with protective barriers of radiation-absorbing material selected from the group consisting of aluminum, plastics, wood, lead, iron, lead glass, water, rubber, plastic, cloth, devices ensuring adequate distances from the radiation sources, devices reducing exposure time to the radionuclide, devices restricting inhalation, ingestion, or other modes of entry of radioactive material into the body and devices providing combinations of these measures.

In some embodiments, an administration device is selected from the group of syringes, shielded syringes, needles, pumps, and infusion devices and combinations thereof. Syringe shields are commonly hollow cylindrical structures that accommodate the cylindrical body of the syringe and are constructed of lead or tungsten with a lead glass window that allows the handler to view the syringe plunger and liquid volume within the syringe.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Abbreviations used in the instant application and the following examples in particular are as follows:

| | |
|---|---|
| 2-PMPA | means 2-(Phosphonomethyl)pentanedioic acid |
| ACN | means acetonitrile |
| AF488 | means Alexa Fluor 488 Dye |
| Alloc | means allyloxycarbonyl |
| ATCC | means American Type Culture Collection |
| AUC | means area under the curve |
| Boc | means tert-butyloxycarbonyl |
| bps | means blood pool serogate, refers to a ROI |
| | drawn around the heart ventricle |
| C4-2 | means human prostate cancer cell line with |
| | the ATCC identifier CRL-3314 |
| CM | means ChemMatrixTM |
| DAD | means diode array detector/detection |
| DBCO | means dibenzocyclooctin |
| DBU | means 1,8 diazabicyclo(5.4.0)undec-7-ene |
| DCM | means dichloromethane |
| Dde | means N-(1-(4,4-dimethyl-2,6-dioxocyclohexylidene)ethyl) |
| DEG | means PS crosslinked with di-ethylene-glycol- dimethacrylate |
| DIAD | means diisopropyl azodicarboxylate |
| DIC | means diisopropyl carbodiimide |
| DIPEA | means N,N-diisopropylethylamine |
| DMF | means N,N-dimethylformamide |
| DMSO | means dimethyl sulfoxide |
| DOTA | means 1,4,7,10-tetraazacyclododecane-1,4,7,10-tetraacetic acid |
| DOTAGA | means 1,4,7,10-tetraazacyclododececane,1-(glutaric acid)- |
| | 4,7,10-triacetic acid |
| DTPA | means diethylenetriaminepentaacetic acid |
| EDT | means 1,2-ethanedithiol |
| eq. | means equivalents |
| ESI | means electrospray ionization |
| FACS | means fluorescence-activated cell sorting |
| Fc | means fragment crystallizable region of an antibody |
| Fmoc | means 9-fluorenylmethoxycarbonyl |
| FMPB | means 4-formyl-3-methoxy-phenoxybutyl |
| GCPII | means Glutamate Carboxypeptidase II |
| HATU | means O-(7-azabenzotriazol-1-yl)-N,N,N′,N′-tetramethyluronium |
| | hexafluoro-phosphate |
| HBST | means 10 mM HEPES pH 7.4, 150 mM NaCl, 0.005% Surfactant P20 |
| HEPES | means 4-(2-hydroxyethyl)-1-piperazineethanesulfonic acid |
| HFIP | means hexafluoro-2-propanol |
| HOAc | means acetic acid |
| HPLC | means high-performance liquid chromatography |
| HPLC/MS | means high-performance liquid chromatography mass spectrometry |
| hPSMA-FC | means human PSMA fused to Fc |
| IC50 | means half-maximal inhibitory concentration |
| IDBS | means the company ID Business Solutions Ltd |
| ivDde | means 1-(4,4-dimethyl-2,6-dioxocyclohex-1-ylidene)-3-methylbutyl |
| K_{D} | means dissociation constant |
| LC | means liquid chromatography |
| LC/Q-TOF-MS | means liquid chromatography quadrupole time-of-flight mass |
| | spectrometry |
| LC/TOF-MS | means liquid chromatography time-of-flight mass spectrometry |
| LC-MS | means liquid chromatography mass spectrometry |
| M | means molar, i.e. mol per Liter |
| m/z | means mass divided by charge |
| MBq | means megabecquerel |
| MeOH | means methanol |
| MFI | means median fluorescence intensities |
| min | means minute(s) |
| MS | means mass spectrometry |
| MTBE | means methyl-tert-butylether |
| MW | means molecular weight |
| NHS | means N-hydroxysuccinimide |
| NMP | means N-methyl-2-pyrrolidone |
| NMRI nude | means Naval Medical Research Institute nude mice |
| mice | |
| PBS | means phosphate buffered saline |
| PC3 | means human prostate cancer cell line with the |
| | ATCC identifier CRL-3314 |
| PC3-PIP | means PSMA transfected PC3 cells developed by |
| | J. B. Latouche and M. Sadelain |
| PE | means polyethylene |
| PET | means Positron Emission Tomography |
| p.i. | means post injection |
| pIC50 | means the negative decadic logarithm of the |
| | IC50 value when converted to molar |
| pKD | means the negative decadic logarithm of the |
| | KD value when converted to molar |
| ppm | means parts per million |
| PS | means polystyrene |
| PSMA | means glutamate carboxypeptidase 2 |
| RFU/sec | means relative fluorescence units per seconds |
| RP | means reversed phase |
| RPMI-1640 | means cell culture growth medium RPMI-1640 |
| RT | means room temperature |
| RU | means relative response units |
| SCK | means single cycle kinetic |
| SDmp | means 2,6-dimethoxyphenyl-1-thio |
| SPE | means solid phase extraction |
| SPECT | means Single Photon Emission Computed Tomography |
| SPPS | means solid phase peptide synthesis |
| SPR | means surface plasmon resonance |
| t_{1/2} | means half-life |
| t_{R} | means retention time |
| tBu | means tert. Butyl |
| TCEP | means tris(2-carboxyethyl)phosphane |
| TFA | means trifluoroacetic acid or trifluoroacetate |
| THF | means tetrahydrofuran |
| TIPS | means triisopropylsilane |
| TOF | means time-of-flight detection |
| UV | means ultraviolet |

### Example 1: Materials, Instruments and Methods

The materials and methods as well as general methods are further illustrated by the following examples.

### Materials:

### Solvents:

Solvents were used in the specified quality without further purification. Acetonitrile (Super Gradient, HPLC, VWR—for analytical purposes; PrepSoly, Merck—for preparative purposes); dichloromethane (synthesis grade, Roth); dimethylsulfoxide (for preparative HPLC: BioScience Grade, Roth—for synthesis: pure, Thermo Scientific), ethyl acetate (synthesis grade, Roth); N,N-dimethylformamide (peptide synthesis grade, Biosolve); 1-methyl-2-pyrolidone (peptide grade, IRIS BioTech); 1,4-dioxane (reinst, Roth); methanol (p. a., Merck); cyclohexane (synthesis, Roth); methyl-tert-butylether (synthesis grade, Roth); water (Milli-Q Plus, Millipore, demineralized).

### Chemicals:

Chemicals were either synthesized according to or in analogy to literature procedures or purchased from Sigma-Aldrich-Merck (Deisenhofen, Germany), Bachem (Bubendorf, Switzerland), VWR (Darmstadt, Germany), Novabiochem (Merck Group, Darmstadt, Germany), Acros Organics (distribution company Fisher Scientific GmbH, Schwerte, Germany), Iris Biotech (Marktredwitz, Germany), Amatek Chemical (Jiangsu, China), Roth (Karlsruhe, Deutschland), Molecular Devices (Chicago, IL, USA), Biochrom (Berlin, Germany), Peptech (Cambridge, MA, USA), Synthetech (Albany, OR, USA), Pharmacore (High Point, NC, USA), PCAS Biomatrix Inc (Saint-Jean-sur-Richelieu, Quebec, Canada), Alfa Aesar (Karlsruhe, Germany), Tianjin Nankai Hecheng S&T Co., Ltd (Tianjin, China), CheMatech (Dijon, France), JenKem Technology (Plano, TX, USA), Trimen Chemicals (Lodz, Poland) and Anaspec (San Jose, CA, USA) or other companies and used in the assigned quality without further purification.

### Solid Phase Synthesis Resins and Resin Linkers:

Solid-phase synthesis was performed on polystyrene resin (PS—polystyrene cross-linked with 1,4-divinylbenzene or DEG—polystyrene cross-linked with di-ethylene-glycol-dimethacrylate) or ChemMatrix resin (CM) modified with a Rink amide, 2-chloro-trityl, 4-methyltrityl or FMPB (4-formyl-3-methoxy-phenoxybutyl) linker. For the synthesis of peptide alcohols pre-loaded 2-Chloro-Trityl resin (PS) was used.

### Instruments and Instrument-Methods:

### HPLC/MS Analyses:

HPLC/MS analyses were performed by injection of 5 μl of a solution of the sample, using a 2-step gradient for all chromatograms (5-65% B in 12 min, followed by 65-90% B in 0.5 min, A: 0.1% TFA in water and B: 0.1% TFA in ACN). RP columns were purchased from Dr. Maisch (ReproSil-Pur 120 C18-AQ, 3 μm, 50×3.00 mm, flow 0.8 mL, HPLC at room temperature); Mass spectrometer: Agilent 6230 LC/TOF-MS or Agilent 6530 LC/Q-TOF-MS, ESI ionization. MassHunter Qualitative Analysis B.07.00 SP2 was used as software. UV detection was done at λ=230 nm. Retention times (Rₜ) are indicated in the decimal system (e.g. 1.9 min=1 min 54 s) and are referring to detection in the UV spectrometer. For the evaluation of observed compound masses the ‘Find Compounds by Formula’-feature was used. More precisely, the individual ‘neutral mass of a compound (in units of Daltons)’-values and the corresponding isotope distribution pattern were used to confirm compound identity. The accuracy of the mass spectrometer was approx. 5 ppm.

### Product Purification Methods—Preparative HPLC.

Preparative HPLC separations were performed on reversed phase columns (General: Kinetex 5μ XB—C18 100 Å, 150×30 mm from Phenomenex) as stationary phase. 0.1% TFA in water (A) and 0.1% TFA in ACN (B) were used as mobile phase which were mixed in linear binary gradients. The gradients are described as: “10 to 40% B in 30 min”, which means a linear gradient from 10% B (and correspondingly 90% A) to 40% B (and correspondingly 60% A) was run over 30 min. Flow-rates were within the range of 30 to 50 mL/min. A typical gradient for the purification of the compounds of the invention started at 5-25% B and ended after 30 min at 35-50% B. The difference between the percentage of B at end and start was at least 10%. A commonly used gradient was “15 to 40% B in 30 min”. Samples were preferably dissolved in mixtures of HOAc and water or DMSO.

### Product Purification Methods—Solid Phase Extraction (SPE):

In case of solid phase extraction, 250 mg Varian Bondesil-ENV was placed in a 15 mL polystyrene syringe. The column was pre-washed with methanol (1×5 mL) and water (2×5 mL) before the reaction solution or the solution containing the product to be purified was applied to the column. To remove excess salt, the column was washed again with water (2×5 mL. Afterward, the product was eluted with 5 mL of 50% ACN in water (first fraction) followed by at least 5 mL of 50% ACN in water containing 0.1% TFA or until elution of the compound was complete.

### Automated/Semi-Automated Solid-Phase Synthesis Equipment:

Automated solid-phase synthesis of peptides and polyamides was performed on a Tetras Peptide Synthesizer (Advanced ChemTech) in 25 μmol, 50 μmol or 100 μmol scales. Manual steps were performed in plastic syringes equipped with frits (material PE, Roland Vetter Laborbedarf OHG, Ammerbuch, Germany). Coupling at elevated temperatures was performed on a Chorus Peptide Synthesizer (Gyros Protein Technologies)—in case of syntheses where couplings with elevated temperatures were part of the sequence assembly, usually the whole sequence was synthesized on the Chorus Peptide Synthesizer.

### General Procedures for Automated/Semi-Automated Solid-Phase Synthesis

The amount of reagents in the protocols described corresponds to the 100 μmol scale, unless stated otherwise.

### Resin Loading—Rink Amide Linker/Sieber Amide Linker (C-Terminal Primary Amides):

For the synthesis of C-terminal peptide amides (primary amides) the Rink amide linker (on CM or DEG resin—initial resin loading ranging from 0.4-0.6 mmol/g) was used. For the synthesis of protected C-terminal peptide amide fragments the Sieber amide linker (on PS resin—initial loading 0.57 mmol/g) was used. The resin was initially swollen in DMF (5 mL) for at least 30 minutes and subsequently washed with DMF (3 mL, 1 minute). The first building block was loaded onto the linker by performing the procedure for the coupling of amino acid building blocks as described below.

### Resin Loading—2-Chloro Trityl Linker (C-Terminal Acids):

For the synthesis of C-terminal peptide acids, especially for the synthesis of protected C-terminal peptide acids fragments, the 2-chloro trityl linker (on PS resin—initial resin loading 1.8 mmol/g) was used. The resin was initially swollen in DCM (5 mL) for at least 30 minutes and subsequently washed with DCM (3 mL, 1 minute). Then the Fmoc amino acid building block was loaded onto the linker by treating the resin for 1 hour with a mixture of the corresponding Fmoc amino acid building block (0.5 mmol, 5 eq.) and DIPEA (350 μL, 3.5 mmol, 35 eq.) in DCM (4 mL). Afterwards, the resin was washed with methanol (5 mL, 5 minutes) and DMF (3 mL, 2×1 minute).

### Resin Loading—4-Methyl Trityl Linker (C-Terminal Amines):

For the synthesis of peptide amines (with a primary amine), the 4-methyl trityl linker (on PS resin—initial resin loading 1.3-1.7 mmol/g) was used. The resin was initially swollen in DCM (5 mL) for at least 30 minutes and subsequently washed with DCM (3 mL, 1 minute). A symmetrical amine (e.g. ethylene diamine) was loaded onto the linker by treating the resin for 1 hour with a mixture of the corresponding amine (0.5 mmol, 5 eq.) and DIPEA (350 μL, 3.5 mmol, 35 eq.) in DCM (4 mL). Afterwards the resin was washed with methanol (5 mL, 5 minutes) and DMF (3 mL, 2×1 minute).

### Resin Loading—FMPB Linker (C-Terminal Secondary Amides):

For the synthesis of C-terminal peptide amides (secondary amides) the FMPB linker (4-Formyl-3-Methoxy-Phenoxybutyl on PS resin—initial resin loading ranging from 0.7-1.0 mmol/g) was used. The resin was initially swollen in DMF (5 mL) for at least 30 minutes and subsequently washed with DMF (3 mL, 1 minute). The amine (0.5 mmol, 10 eq.—e.g. n-butylamine) was dissolved in 1% acetic acid in DMF (1.5 mL). After agitation of the resin for 5 minutes, NaBH₃CN (0.5 mmol, 10 eq.) was added and the resin was agitated at 50° C. overnight. The resin was washed with DMF, MeOH and DMF (3 mL each, 3×1 minute). The first sequence building block was then coupled following the procedure for carboxylic acid building blocks at 50° C. and with an extended coupling time of 2 hours. The coupling was repeated once.

Coupling of Carboxylic Acid Building Blocks: (e.g. Fmoc Amino Acids)

Solutions of reagents: Building Blocks (0.3 M in DMF or NMP), DIPEA (0.9 M in DMF), HATU (0.4 M in DMF), acetic anhydride (0.75 M in DMF), DIC (3.2 M in DMF).

Unless otherwise stated, coupling of amino acid building blocks or carboxylic acids in general was performed as follows: After subsequent addition of solutions of the corresponding carboxylic acid building block (0.9 mL, 5 eq.), DIPEA solution (0.6 mL, 10 eq.) and HATU solution (0.65 mL, 5 eq.) to the resin, the latter was shaken for 45 min at room temperature. Afterwards the resin was washed with DMF (3 mL, 1 minute). If necessary, the coupling step was repeated. The above description of coupling of carboxylic acid building blocks applies to a 50 μmol synthesis.

Coupling of Chelators Building Blocks: (e.g. DOTA or DOTAGA)

The corresponding chelator building block (DOTA(tBu)₃-OH or (DOTAGA(tBu)₄-OH) was coupled as described for the coupling of carboxylic acid building blocks but coupling time was increased to 90 min. Furthermore, after this timespan a DIC solution (3.2 M in DMF, 0.2 mL, 12.5 eq.) was added and the resin agitated for further 90 minutes.

### N-Terminal Acetylation:

After addition of DIPEA solution (1.75 mL, 16 eq.) and acetic anhydride solution (1.75 mL, 13 eq.) to the resin, the latter was shaken for 10 minutes. Afterwards the resin was washed with DMF (3 mL, 6×1 minutes).

N-Terminal Attachment of Urea Moieties: (e.g. n-Butyl Urea)

After the mixture of a corresponding isocyanate (e.g. n-butylisocyanate) (0.5 mmol, 5 eq.) and DIPEA (1 mmol, 10 eq.) in DMF (3 mL) to the resin, the latter is agitated for 2 hours. Afterwards the resin is washed with DMF (3 mL, 1 minute).

### Coupling of DOTA-NHS in Solution:

For the coupling of DOTA to a peptide precursor in solution, the peptide was dissolved in a minimal volume of DMSO and the pH value of the resulting solution was set to 8.0-8.5 by careful addition of small volumes of DIPEA. A solution of DOTA-NHS (1.3 eq. in relation to the amount of peptide) in a minimal amount/volume of DMSO was added to the solution. The reaction was monitored by LC-MS after approximately 1 hour reaction time. If necessary, more DOTA-NHS was added or the pH value re-adjusted to 8.0-8.5. After completion of the reaction, the solution was either directly subjected to purification via Preparative HPLC or used for the next reaction step. For the optional removal of a Dde/ivDde protecting group, hydrazine hydrate (final concentration of hydrazine 2%, e.g. 10 μL of hydrazine hydrate were added to 500 μL of a DMSO solution) was added to the reaction mixture. Usually the deprotection of Dde/ivDde was finished after 10 minutes. The solution was then acidified by addition of TFA (10 μL TFA were added to 500 μL reaction solution) and was directly submitted to purification via Preparative HPLC.

### Fmoc Deprotection:

After swelling in DMF, the resin was washed with DMF, treated with piperidine/DMF (1:4, 3 mL, 2 and 20 minutes) and subsequently washed with DMF (3 mL, 5×1 minute).

### Alloc/Allyl Deprotection:

After swelling in DMF, the resin was washed with DMF and DCM. DCM was de-oxygenated by passing a stream of nitrogen through the stirred solvent. The oxygen-free solvent was used to wash the resin trice. Then 2 mL of a 2 M solution of barbituric acid in oxygen-free DCM and 1 mL of a 25 μM solution of tetrakis(triphenylphosphine)palladium(0) in oxygen-free DCM were added to the resin. The resin was agitated for 1 hour and then washed with DCM, MeOH, DMF, 0.5% DIPEA in DMF, 0.5% dithiocarbamate in DMF, DMF and DCM (each washing step was repeated 3 times with 3 mL, 1 minute).

### Dde/ivDde Deprotection:

After swelling in DMF, the resin was washed with DMF, treated with hydrazine-hydrate/DMF (2/98, 3 mL 2×10 minutes) and subsequently washed with DMF (3 mL, 5×1 minute).

### SDmp Deprotection:

After swelling in DMF, the resin was washed with DMF, treated with a solution of 20% β-mercaptoethanol in 0.1 M N-methylmorpholine in DMF (3 mL, 3×5 minutes) and subsequently washed with DMF (3 mL, 5×1 minute).

### Selective N-Methylation Under Mitsunobu Conditions:

The resin was swollen or thoroughly washed with DCM. The amino group was protected with a nosyl group by treating the resin with a solution of 2-nitrobenzenesulfonyl chloride (4 eq.) and sym-collidine (10 eq.) in DCM for 30 min. The resin was washed with DCM, THF and finally dry THF. After adding a solution of triphenylphosphane (10 eq.) and MeOH (dry, 20 eq.) in THF (dry), the solution of diisopropyl azodicarboxylate (DIAD) (10 eq.) in THF (dry) was added to the resin. After 30 minutes the resin was washed with THF and THF (dry) and the procedure repeated once. The resin was washed with DMF and the nosyl protecting group finally removed by treating the resin with a solution of β-mercapoethanol (10 eq.) and 1,8-diazabicyclo(5.4.0)undec-7-ene (DBU) (5 eq.) in DMF for 30 minutes. Finally, the resin was washed with DMF.

### Solid Phase Peptide Synthesis (SPPS):

After loading of the C-terminal/initial building block onto the resin linker of a resin, the linear sequence of a peptide was assembled by iterative repetition of Fmoc deprotection and Coupling of carboxylic acid building blocks (Fmoc amino acid building blocks).

Dependent on the desired structure of the target peptide the N-terminus was
a free amine (no further action after the final ‘Fmoc deprotection’),
a chelator (DOTA or DOTAGA), which was attached by employing the ‘Coupling of chelators building blocks’ method,
an acetyl group, which was attached by employing the ‘N-terminal acetylation’ method,
a carboxylic acid such as hexanoic acid, which was attached by employing the ‘Coupling of carboxylic acid building blocks’ method or
an urea moiety such as n-butyl urea, which was attached by employing the ‘N-terminal attachment of urea moieties’ method.
   Cleavage Method A: Cleavage of Protected Fragments from Hyper-Acid Labile Resin:

After completion of the assembly of the sequence the resin was finally washed with DCM (3 mL, 4×1 minute) and then dried in the vacuum. Then, the resin was treated with HFIP/DCM (7/1, 4 mL, 4 hours) and the collected solution evaporated to dryness. The residue was purified by preparative HPLC or used without further purification.

Cleavage Method B: Cleavage of Protected Fragments from Hyper-Acid Labile Resin:

After completion of the assembly of the sequence, the resin was finally washed with DCM (3 mL, 4×1 minute) and then dried in the vacuum. Then, the resin was treated with a solution of TFA, TIPS and DCM (1/2.5/96.5—4 mL—10×2 minutes). Individual cleavage fractions were poured into MeOH which had been set to a neutral pH value by addition of DIPEA. After the final treatment of the resin with dilute acid, it was washed with MeOH and DCM. All cleavage fractions and washing solutions were combined and concentrated under reduced pressure. Ice-water was added to the remaining solution and the precipitated crude product obtained by centrifugation. The resulting residue was typically used in the next step without an intermediate purification.

### Cleavage Method C: Cleavage of Unprotected Fragments (Complete Resin Cleavage):

After completion of the assembly of the sequence, the resin was finally washed with DCM (3 mL, 4×1 minute), dried in the vacuum overnight and treated with TFA, EDT, water and TIPS (94/2.5/2.5/1-4 mL) for 4 h (unless otherwise stated). Afterwards, the cleavage solution was poured into a chilled mixture of MTBE and cyclohexane (1/1, 10-fold excess compared to the volume of cleavage solution), centrifuged at 4° C. for 5 minutes. The residue was lyophilised from water/acetonitrile prior to purification or further modification.

### Cleavage Method D: Cleavage of Protective Groups of Peptides in Solution:

The protected/partially protected compound was dissolved in TFA, water and TIPS (95/2.5/2.5-4 mL) and agitated for 2 hours (unless otherwise stated). Afterwards, the cleavage solution was poured into a chilled mixture of MTBE and cyclohexane (1/1, 10-fold excess compared to the volume of cleavage solution), centrifuged at 4° C. for 5 minutes. The residue was lyophilised from water/acetonitrile prior to purification or further modification.

### Cyclization Method A: Disulfide Cyclization:

The crude peptide material was dissolved in a 1:1 mixture of acetonitrile and ammonium acetate buffer (0.1 M, pH 6). To the solution [Pt(en)₂Cl₂]Cl₂ (Dichlorobis-(ethylendiamine)-platinum(IV) chloride) was added. Upon completion of the cyclization reaction which was judged by analytical LC-MS, TFA was added and the reaction solution subjected to lyophilisation. The volume of solvent, amount of Pt-reagent and volume of TFA used in the reaction depended on the amount of resin used for the synthesis of the linear peptide precursor—per 50 μmol of initially used resin 60 mL of the solvent mixture, 22.8 mg (50 μmol) of Pt-reagent and 50 μL of TFA were used.

Cyclization Method B: Cyclization with Diodo Methane:

The crude peptide material (50 μmol) was dissolved in a water (10 mL) and THF (5 mL). To this solution K₂CO₃ (300 μmol, 6 eq.) and TCEP (75 μmol, 1.5 eq.) were added. After 5 minutes, triethylamine (250 μmol, 5 eq.) and diodomethane (400 μmol, 8 eq.) were added and the solution was stirred at 50° C. for 45 minutes. β-Mercaptoethanol (60 μL) was added and the mixture left to stir for additional 45 minutes. Then, TFA (50 μL) and acetonitrile (10 mL) were added and the mixture was subjected to lyophilisation.

Synthesis of Acetylated Guanidin Side Chains (e.g. Acetylated Arginines):

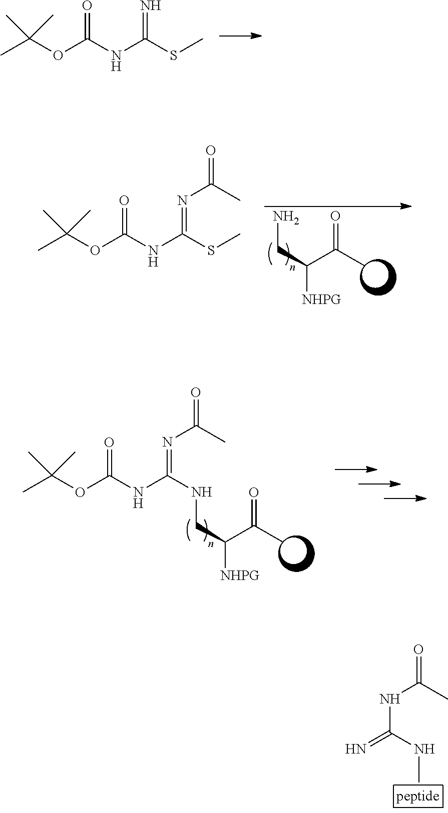

For the synthesis of 50 μmol peptide, 1-N-Boc-2-methyl-isothiourea (325 μmol, 6.5 eq.), acetic acid anhydride (325 μmol, 6.5 eq.) and DIPEA (650 μmol, 13 eq.) were dissolved in DCM (1 mL) and stirred for 6 hours. Then the volatiles were removed in the vacuum and the remainder re-dissolved in DMF (2 mL). Before addition of the solution to the peptide resin with a specifically deprotected amine, DIPEA (600 μmol, 12 eq.) was added to the solution. The resin was agitated for 4 hours and then washed with DMF (3 mL, 3×1 minute).

Synthesis of Carbamoylated Guanidine Side Chains I (e.g. Ethyl Carbamoyl Arginines):

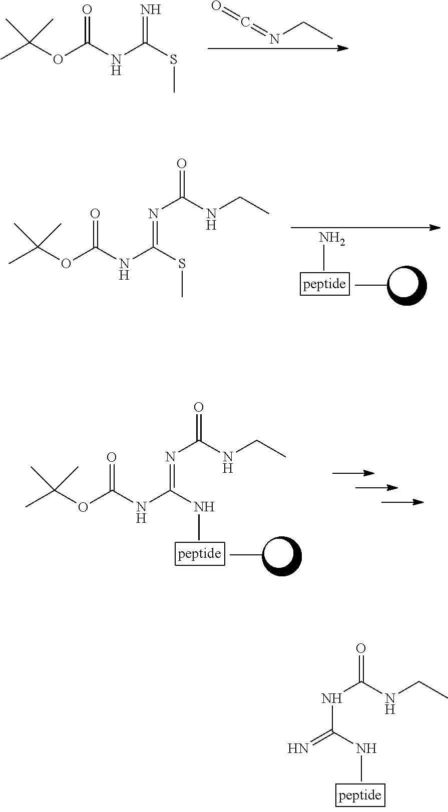

For the synthesis of 50 μmol peptide, the solution of 1-N-Boc-2-methyl-isothiourea (250 μmol, 5 eq.) and ethylisocyanate (250 μmol, 5 eq.) in DCM (1 mL) was stirred overnight. The solution was then added to the resin. After addition of a solution of mercury(II)chloride (300 μmol, 6 eq.) and DIPEA (200 μmol, 4 eq.) in DCM (35 μL) the resin was agitated for 4 hours and then washed with DCM, MeOH, DMF, 0.5% DIPEA in DMF, 0.5% dithiocarbamate in DMF, DMF and DCM (each washing step was repeated 3 times with 3 mL, 1 minute). The transformation was performed as final step before resin cleavage.

Synthesis of Carbamoylated Guanidine Side Chains II (e.g. 3-keto-4-aza-arginine):

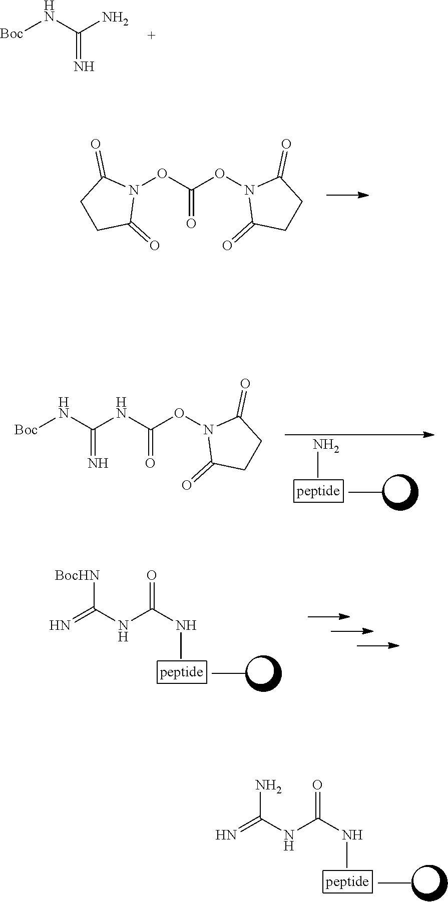

For the synthesis of 50 μmol peptide, N,N-disuccinimidyl carbonate (250 μmol, 5 eq.) and Boc-guanidine (275 μmol, 5.5 eq.) were dissolved in DCM (3 mL). After stirring for 2 hours the solution was transferred to the resin, which had previously been swollen in DMF/DCM (1:1). Then DIPEA (250 μmol, 5 eq.) was added and the resin was agitated for 6 hours. The resin was washed with DMF (3 mL, 3×1 minute) and DCM (3 mL, 3×1 minute). The transformation was performed as final step before resin cleavage.

Synthesis of Acyl Guanidine Side Chains (e.g. Glutamine/Glutamate Guanidines):

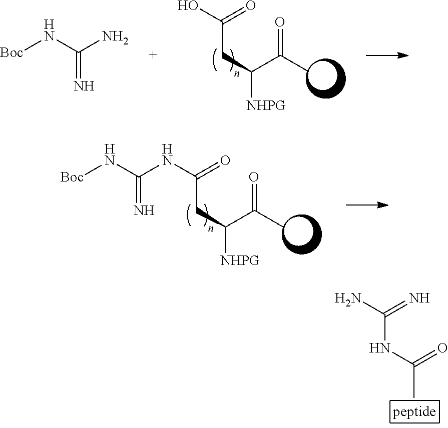

For the synthesis of 50 μmol peptide, Boc-guanidine (250 μmol, 5 eq.), N-morpholine (350 μmol, 7 eq.) and HATU (75 μmol, 1.5 eq.) were dissolved in DMF (0.9 mL). The resin was agitated overnight and then washed with DMF (3 mL, 3×1 minute).

### Synthesis of Alkylated Guanidine Side Chains (e.g. δ,ω-Dimethyl Arginine):

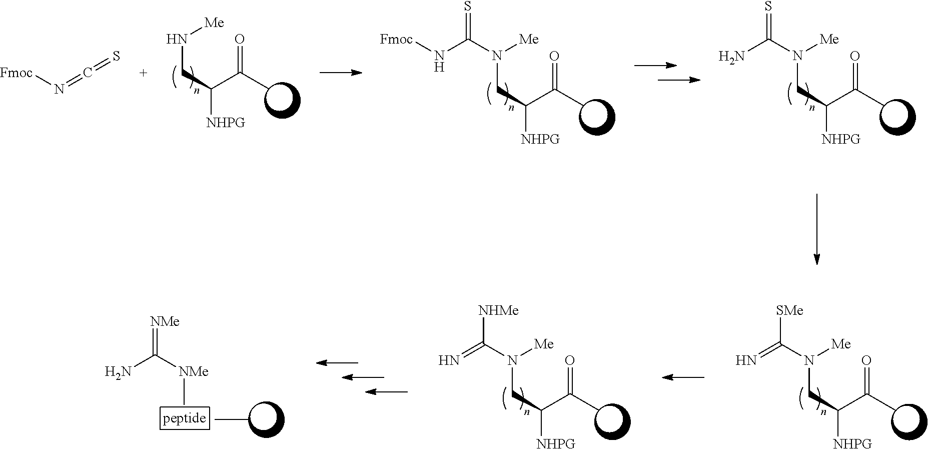

The peptide resin (with a selectively deblocked amino function) was thoroughly washed with DCM and treated overnight with a solution of an isothiocyanate (10 eq.) in DCM. In case of an Fmoc-protected isothiocyanate, the Fmoc group was removed by implementing an ‘Fmoc deprotection’ step. The resin was then treated with a solution of methyliodide in DMF (0.2 M) for 1 hour, which was repeated three times to achieve the methylation of the sulphur atom. The reaction sequence was concluded by treating the resin with a solution of an amine (e.g. methylamine 2.0 M) in THF overnight.

### Strain-Promoted Click Reaction:

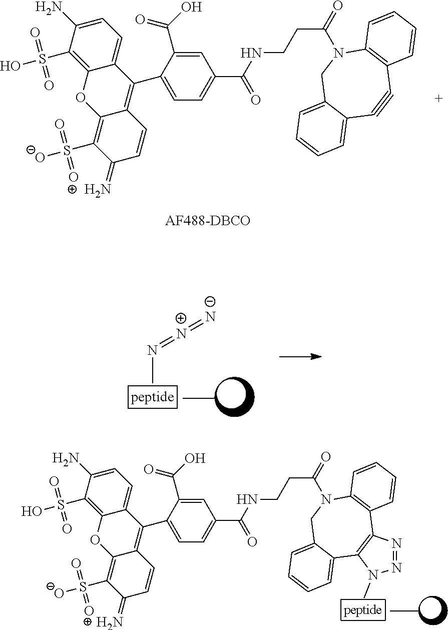

To a solution of the purified azido peptide (1.26 μmol, 1 eq.) in water (150 μL) and acetonitrile (50 μL), a solution of AF488-DBCO (1.26 μmol, 1 eq.) in water (60 μL) and acetonitrile (20 μL) was added. The solution was agitated overnight and subjected to lyophilisation.

More relevant Fmoc-solid-phase-peptide synthesis methods are described in detail in “Fmoc Solid Phase Peptide Synthesis” Editors W. Chan, P. White, Oxford University Press, USA, 2000. Compounds were named using MestreNova version 12 Mnova IUPAC Name plugin (Mestrelab Research, S.L.), or AutoNom version 2.2 (Beilstein Informationssysteme Copyright© 1988-1998, Beilstein Institut für Literatur der Organischen Chemie licensed to Beilstein Chemiedaten and Software GmbH), where appropriate.

### Preparation of Compounds:

Specific embodiments for the preparation of compounds of the invention are prepared by using the preferred general methods disclosed above, other published methods or methods known by persons skilled in the art. Unless otherwise specified, all starting materials and reagents are either of standard commercial grade and are used without further purification or are readily prepared from such materials by routine methods. Those skilled in the art of organic synthesis will recognize in light of the instant disclosure that starting materials and reaction conditions may be varied including additional steps employed to produce compounds encompassed by the present invention.

### Example 2: Synthesis I: Synthesis of Compound Ac-Thr-Aib-Phe-[Cys-Lys-Arg-Ala-Asn-Cys]-Tle-Thr-Asp-NH₂ (PSM-0565)

The linear sequence of the peptide (Ac-Thr-Aib-Phe-Cys-Lys-Arg-Ala-Asn-Cys-Tle-Thr-Asp-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. After cleavage of the linear peptide from the synthesis resin employing ‘Cleavage method C: Cleavage of unprotected fragments (complete resin cleavage)’ (cleavage time 2 hours) the obtained crude material was subjected to ‘Cyclization method A: Disulfide cyclization’. After lyophilisation of the reaction solution the crude product was purified by ‘Preparative HPLC’ (15 to 30% B in 30 min—Kinetex) to yield 11.99 mg of the pure title compound (17.2%). HPLC t_{R}=4.09 min. LC/TOF-MS: exact mass 1394.665 (calculated 1394.644). C₅₈H₉₄N₁₈O₁₈S₂ (MW=1395.611).

### Example 3: Synthesis II: Synthesis of Compound Ac-Thr-Aib-Phe-[Cys-Lys-Arg-Aib-Asn-Cys]-Tle-Thr-Cmp-lys(DOTA)-NH₂ (PSM-0428)

The linear sequence of the peptide (Ac-Thr-Aib-Phe-Cys-Lys-Arg-Aib-Asn-Cys-Tle-Thr-Cmp-lys(Alloc)-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. After removal of the Alloc protecting group from the C-terminal D-lysine employing an ‘Alloc/Allyl deprotection’, DOTA was coupled employing the ‘Coupling of chelators building blocks’ method. Afterwards the linear, branched peptide (Ac-Thr-Aib-Phe-Cys-Lys-Arg-Aib-Asn-Cys-Tle-Thr-Cmp-lys(DOTA)-NH₂) was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution the crude product was purified by ‘Preparative HPLC’ (10 to 30% B in 30 min—Kinetex) to yield 16.95 mg of the pure title compound (17.5%). HPLC t_{R}=4.23 min. LC/TOF-MS: exact mass 1933.101 (calculated 1932.991). C₈₄H₁₄₀N₂₄O₂₄S₂ (MW=1934.292).

### Example 4: Synthesis III: Synthesis of Compound Hex-Thr-Aib-Pcf-[Cys-Lys-Arg-Aib-Asn-Cys]-Tle-Thr-lys(DOTA-Cmp)-NH₂ (PSM-0400)

The linear sequence of the peptide (Hex-Thr-Aib-Pcf-Cys-Lys-Arg-Aib-Asn-Cys-Tle-Thr-lys(Alloc)-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with coupling of hexanoic acid as final step. After removal of the Alloc protecting group from the C-terminal D-lysine employing an ‘Alloc/Allyl deprotection’, Fmoc-Cmp-OH was coupled employing the ‘Coupling of carboxylic acid building blocks’ method followed by ‘Fmoc deprotection’. Then, DOTA was coupled employing the ‘Coupling of chelators building blocks’ method. Afterwards, the linear, branched peptide (Hex-Thr-Aib-Pcf-Cys-Lys-Arg-Aib-Asn-Cys-Tle-Thr-lys(DOTA-Cmp)-NH₂) was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution, the crude product was purified by ‘Preparative HPLC’ (20 to 40% B in 30 min—Kinetex) to yield 13.52 mg of the pure title compound (13.4%). HPLC t_{R}=5.9 min. LC/TOF-MS: exact mass 2023.036 (calculated 2023.015). C₈₈H₁₄₇ClN₂₄O₂₄S₂ (MW=2024.844).

### Example 5: Synthesis IV: Synthesis of Compound nBuCAyl-Thr-Aib-Pcf-[Cys-Lys-Cit-Aib-Asn-Cys]-Tle-Thr-lys(DOTA-Cmp)-NH₂ (PSM-0500)

The linear sequence of the peptide (H-Thr-Aib-Pcf-Cys-Lys-Cit-Aib-Asn-Cys-Tle-Thr-lys(Alloc)-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’. As final step of the assembly of the linear peptide sequence an n-butyl urea moiety was attached by employing the ‘N-terminal attachment of urea moieties’ method. Then, the Alloc protecting group was removed from the C-terminal D-lysine employing an ‘Alloc/Allyl deprotection’, Fmoc-Cmp-OH was coupled employing the ‘Coupling of carboxylic acid building blocks’ method followed by ‘Fmoc deprotection’. Then, DOTA was coupled employing the ‘Coupling of chelators building blocks’ method. Afterwards, the linear, branched peptide (nBuCAyl-Thr-Aib-Pcf-Cys-Lys-Cit-Aib-Asn-Cys-Tle-Thr-lys(DOTA-Cmp)-NH₂) was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution, the crude product was purified by ‘Preparative HPLC’ (20 to 40% B in 30 min—Kinetex) to yield 17.42 mg of the pure title compound (17.2%). HPLC t_{R}=5.82 min. LC/TOF-MS: exact mass 2024.978 (calculated 2024.994). C₈₇H₁₄₅ClN₂₄O₂₅S₂ (MW=2026.817).

### Example 6: Synthesis V: Synthesis of Compound DOTA-Pamb-Aib-Pcf-[Cys-Lys-Arg-Aib-Asn-Cys]-Tle-en (PSM-0273)

Starting from a PS resin with a 2-chloro trityl linker to which ethylene diamine was loaded the linear sequence of the peptide (DOTA-Pamb-Aib-Pcf-Cys-Lys-Arg-Aib-Asn-Cys-Tle-en) was assembled according to the ‘General procedures for Automated/Semi-automated Solid-Phase Synthesis’ in a 50 μmol scale applying the ‘Solid phase peptide synthesis (SPPS)’ with coupling of DOTA as final building block. The linear peptide precursor was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution the crude product was purified by ‘Preparative HPLC’ (10 to 30% B in 30 min—Kinetex) to yield 22.81 mg of the pure title compound (27.7%). HPLC t_{R}=4.3 min. LC/TOF-MS: exact mass 1645.764 (calculated 1645.762). C₇₁H₁₁₂ClN₂₁O₁₈S₂ (MW=1647.367).

### Example 7: Synthesis VI: Synthesis of Compound Ac-Aib-Pcf-[Cys-Lys-Arg-Aib-Asn-Cys]-Tle-en-DOTA (PSM-0516)

Starting from a PS resin with a 2-chloro trityl linker to which ethylene diamine was loaded the linear sequence of the peptide (Ac-Aib-Pcf-Cys-Lys(Dde)-Arg-Aib-Asn-Cys-Tle-en) was assembled according to the ‘General procedures for Automated/Semi-automated Solid-Phase Synthesis’ in a 50 μmol scale applying the ‘Solid phase peptide synthesis (SPPS)’ with an N-terminal acetylation as final step. The linear peptide precursor was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution the crude intermediate product was purified by ‘Preparative HPLC’ (20 to 40% B in 30 min—Kinetex) to yield 22.27 mg (16.7%) of the intermediate peptide (Ac-Aib-Pcf-[Cys-Lys(Dde)-Arg-Aib-Asn-Cys]-Tle-en). The complete amount of the latter was subjected to the ‘Coupling of DOTA-NHS in solution’ method including the Dde deprotection from the lysine side chain. The reaction solution was then directed to purification by ‘Preparative HPLC’ (10 to 30% B in 30 min—Kinetex) to yield 13.22 mg of the pure title compound (8.5%). HPLC t_{R}=4.25 min. LC/TOF-MS: exact mass 1554.706 (calculated 1554.72). C₆₅H₁₀₇ClN₂₀O₁₈S₂ (MW=1556.256).

### Example 8: Synthesis VII: Synthesis of Compound Ac-Thr-Aib-Phe-[Cys-Lys-Arg-Aib-Asn-Cys]-Tle-OH (PSM-0300)

Starting from a PS resin with a 2-chloro trityl linker to which Fmoc-Tle-OH was loaded the linear sequence of the peptide (Ac-Thr-Aib-Phe-Cys-Lys-Arg-Aib-Asn-Cys-Tle-OH) was assembled according to the ‘General procedures for Automated/Semi-automated Solid-Phase Synthesis’ in a 25 μmol scale applying the ‘Solid phase peptide synthesis (SPPS)’ with an N-terminal acetylation as final step. The linear peptide precursor was cleaved from the synthesis resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution the crude product was purified by ‘Preparative HPLC’ (10 to 30% B in 30 min—Kinetex) to yield 5.06 mg of the pure title compound (16.9%). HPLC t_{R}=4.82 min. LC/TOF-MS: exact mass 1193.568 (calculated 1193.569). C₅₁H₈₃N₁₅O₁₄S₂ (MW=1194.431).

### Example 9: Synthesis VIII: Synthesis of Compound Ac-Aib-Pcf-[Cys-Lys(Me)-Arg-Aib-Asn-Cys]-Tle-en-DOTA (PSM-0472)

Starting from a PS resin with 4-methyl trityl linker to which ethylene diamine was loaded, the linear sequence of the peptide (Ac-Aib-Pcf-Cys(SDmp)-Lys(Me,Boc)-Arg(Pbf)-Aib-Asn(Trt)-Cys(SDmp)-Tle-en) was assembled according to the ‘General procedures for Automated/Semi-automated Solid-Phase Synthesis’ in a 100 μmol scale applying the ‘Solid phase peptide synthesis (SPPS)’ with an N-terminal acetylation as final step. The SDmp protecting groups were removed from the cysteines by employing the ‘SDmp deprotection’ deprotection method and the partially protected peptide fragment detached from the resin employing the ‘Cleavage method A: Cleavage of protected fragments from hyper-acid labile resin’ method. The crude linear intermediate (Ac-Aib-Pcf-Cys-Lys(Me,Boc)-Arg(Pbf)-Aib-Asn(Trt)-Cys-Tle-en was subjected to cyclization using the method ‘Cyclization method A: Disulfide cyclization’ and then directed to a purification employing ‘Solid phase extraction (SPE)’ to yield 14.52 mg (9.5%) of the intermediate peptide. The latter was subjected to the ‘Coupling of DOTA-NHS in solution’ method. The volatiles were removed in the vacuum and the remainder treated with the ‘Cleavage method D: Cleavage of protective groups of peptides in solution’ method. The obtained crude material was purified by ‘Preparative HPLC’ (10 to 30% B in 30 min—Kinetex) to yield 5.54 mg of the pure title compound (3.5%). HPLC t_{R}=4.15 min. LC/TOF-MS: exact mass 1568.735 (calculated 1568.736). C₆₆H₁₀₉ClN₂₀O₁₈S₂ (MW=1570.283).

### Example 10: Synthesis IX: Synthesis of Compound Ac-Thr-Aib-Pcf-[Cys-Nle-Gln(Gu)-Aib-Asn-Cys]-Tle-Thr-NH₂ (PSM-0236)

The linear sequence of the peptide (Ac-Thr-Aib-Pcf-Cys-Nle-Glu(OAll)-Aib-Asn-Cys-Tle-Thr-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. Then the allyl ester protection was removed from the glutamate residue by employing the ‘Alloc/Allyl deprotection’. After the resin was subjected to the ‘Synthesis of acyl guanidine side chains’ method, the peptide (Ac-Thr-Aib-Pcf-Cys-Nle-Gln(Gu)-Aib-Asn-Cys-Tle-Thr-NH₂) was detached from the resin employing ‘Cleavage method C: Cleavage of unprotected fragments (complete resin cleavage)’ and the obtained crude material subjected to cyclization using the method ‘Cyclization method A: Disulfide cyclization’. After lyophilisation of the reaction solution the crude product was purified by ‘Preparative HPLC’ (20 to 40% B in 30 min—Kinetex) to yield 9.14 mg of the pure title compound (13.8%). HPLC t_{R}=6.41 min. LC/TOF-MS: exact mass 1326.561 (calculated 1326.562). C₅₅H₈₇ClN₁₆O₁₆S₂ (MW=1327.964).

### Example 11: Synthesis X: Synthesis of Compound Ac-Thr-Aib-Pcf-[Cys-Lys-Arg(Ac)-Aib-Asn-Cys]-Tle-Thr-NH₂ (PSM-0569)

The linear sequence of the peptide (Ac-Thr-Aib-Pcf-Cys-Lys-Om(Alloc)-Aib-Asn-Cys-Tle-Thr-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. After removal of the Alloc protection group from the ornithine side chain employing an ‘Alloc/Allyl deprotection’, the side chain of the latter was extended to an acetylated arginine side chain by performing the steps of the ‘Synthesis of acetylated guanidin side chains’ method. After cleavage of the linear peptide from the synthesis resin employing ‘Cleavage method C. Cleavage of unprotected fragments (complete resin cleavage)’ (cleavage time 2 hours) the obtained crude material was subjected to ‘Cyclization method A: Disulfide cyclization’. After lyophilisation of the reaction solution the crude product was purified by ‘Preparative HPLC’ (10 to 30% B in 30 min—Kinetex) to yield 1.46 mg of the pure title compound (2.1%). HPLC t_{R}=4.82 min. LC/TOF-MS: exact mass 1369.575 (calculated 1369.604). C₅₇H₉₂ClN₁₇O₁₆S₂ (MW=1371.032).

### Example 12: Synthesis XI: Synthesis of Compound Ac-Thr-Aib-Phe-[Cys-Lys-Arg-Aib-Asn-Cys]-Tle-NHBu (PSM-0510)

For the synthesis of the linear peptide on solid phase in a 50 μmol scale by employing ‘General procedures for Automated/Semi-automated Solid-Phase Synthesis’, initially n-Butyl amine was loaded onto FMPB PS resin. According to the ‘General procedures for Automated/Semi-automated Solid-Phase Synthesis’ the linear sequence of the peptide was assembled applying the ‘Solid phase peptide synthesis (SPPS)’ procedure with the exception that the first building block Fmoc-Tle-OH was coupled twice at 50° C. After completion of the sequence assembly with an acetylation of the N-terminus, the linear peptide (Ac-Thr-Aib-Phe-Cys-Lys-Arg-Aib-Asn-Cys-Tle-NHBu) was cleaved from the resin employing ‘Cleavage method C: Cleavage of unprotected fragments (complete resin cleavage)’ (cleavage time 2 hours) and the obtained crude material subjected to ‘Cyclization method A: Disulfide cyclization’. After lyophilisation of the reaction solution the crude product was purified by ‘Preparative HPLC’ (15 to 35% B in 30 min—Kinetex) to yield 1.99 mg of the pure title compound (3.2%). HPLC t_{R}=5.99 min. LC/TOF-MS: exact mass 1248.645 (calculated 1248.647). C₅₅H₉₂N₁₆O₁₃S₂ (MW=1249.553).

### Example 13 Synthesis XII: DOTA-Cmp-Thr-Aib-Pcf-[Cys-Lys-Arg-Aib-Asn-Cys]-Tle-en(Me) (PSM-0456)

Starting from a PS resin with 2-chloro trityl linker to which Fmoc-Tle-OH was loaded the linear sequence of the peptide (DOTA-Cmp-Thr-Aib-Pcf-Cys-Lys-Arg-Aib-Asn-Cys-Tle-OH) was assembled according to the ‘General procedures for Automated/Semi-automated Solid-Phase Synthesis’ in a 50 μmol scale applying the ‘Solid phase peptide synthesis (SPPS)’ with coupling of DOTA as final building block. The peptide was detached from the solid support employing ‘Cleavage method B: Cleavage of protected fragments from hyper-acid labile resin’. The obtained fully protected peptide fragment (DOTA(OtBu)₃-Cmp-Thr(tBu)-Aib-Pcf-Cys(Trt)-Lys(Boc)-Arg(Pbf)-Aib-Asn(Trt)-Cys(Trt)-Tle-OH—crude mass: 200 mg) was dissolved in DMF (1 mL). To this solution the amine N-(2-aminoethyl)-N-methyl carbamic acid tert-butylester (20 μmol), the activator HATU (20 μmol) and the base DIPEA (30 μmol) were added which resulted in a solution with a pH value of 8.5. The addition of amine and the activator was repeated 3 more times. Afterwards, all volatiles were removed in the vacuum and the reminder lyophilised from water and acetonitrile. The obtained residue was subjected to ‘Cleavage method D: Cleavage of protective groups of peptides in solution’ method. The obtained crude material was purified by ‘Preparative HPLC’ (10 to 30% B in 30 min—Kinetex) to yield 18.91 mg of the pure title compound (21.6%). HPLC t_{R}=3.94 min. LC/TOF-MS: exact mass 1752.852 (calculated 1752.857). C₇₅H₁₂₅ClN₂₂O₂₀S₂ (MW=1754.519).

### Example 14: Synthesis XIII: Synthesis of Compound AF488Ahx-Ttds-Ttds-Thr-Aib-Phe-[Cys-Lys-Arg-Aib-Asn-Cys]-Tle-Thr-Asp-NH₂ (PSM-0272)

The linear sequence of the peptide (N3Ahx-Ttds-Ttds-Thr-Aib-Phe-Cys-Lys-Arg-Aib-Asn-Cys-Tle-Thr-Asp-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’. The solid phase peptide synthesis was complete by attaching 6-azido hexanoic acid (N3Ahx-OH) applying the ‘Coupling of carboxylic acid building blocks’ method. After cleavage of the linear peptide from the synthesis resin employing ‘Cleavage method C: Cleavage of unprotected fragments (complete resin cleavage)’ (cleavage time 2 hours), the obtained crude material was subjected to ‘Cyclization method A: Disulfide cyclization’. After lyophilisation of the reaction solution the crude product was purified by ‘Preparative HPLC’ (15 to 35% B in 30 min—Kinetex) to yield 14.39 mg (9.9%) of the cyclic intermediate peptide (N3Ahx-Ttds-Ttds-Thr-Aib-Phe-[Cys-Lys-Arg-Aib-Asn-Cys]-Tle-Thr-Asp-NH₂). 2.66 mg of the latter were subjected to a ‘Strain-promoted click reaction’ with AF488-DBCO. Purification by ‘Preparative HPLC’ (10 to 30% B in 30 min—Kinetex) yielded 1.51 mg of the pure title compound (41.3%—overall yield 4%). HPLC t_{R}=6.52 min. LC/TOF-MS: exact mass 2902.224 (calculated 2902.211). C₁₃₀H₁₈₃N₂₉O₃₉S₄ (MW=2904.284).

### Example 15: Synthesis XIV: Synthesis of Compound DOTA-Cmp-Thr-Aib-Pcf-[Smc-Lys(Me)-Arg(Me)-Aib-Asn-Cys]-Tle-NH₂ (alternative: DOTA-Cmp-Thr-Aib-Pcf-[Cys-Lys(Me)-Arg(Me)-Aib-Asn-Smc]-Tle-NH₂) (PSM-0420)

The linear sequence of the peptide (DOTA(OtBu)₃-Cmp-Thr(tBu)-Aib-Pcf-Cys(SDmp)-Lys(Me,Boc)-Arg(Me,Pbf)-Aib-Asn(Trt)-Cys(SDmp)-Tle-NH₂) was assembled according to the ‘General procedures for Automated/Semi-automated Solid-Phase Synthesis’ in a 50 μmol scale on a Sieber amide PS resin applying the ‘Solid phase peptide synthesis (SPPS)’ method, with coupling of DOTA as final building block. The SDmp protected groups were removed from the cysteine side chains employing an ‘SDmp deprotection’, prior to the detachment of the partially protected by peptide (DOTA(OtBu)₃-Cmp-Thr(tBu)-Aib-Pcf-Cys-Lys(Me,Boc)-Arg(Me,Pbf)-Aib-Asn(Trt)-Cys-Tle-NH₂) from the solid phase resin by implementation of the ‘Cleavage method B: Cleavage of protected fragments from hyper-acid labile resin’. The obtained crude peptide was directed to ‘Cyclization method B: Cyclization with diodo methane’ and after lyophilisation of the reaction solution further to ‘Cleavage method D: Cleavage of protective groups of peptides in solution’. The resulting crude material was subjected to purification by ‘Preparative HPLC’ (10 to 30% B in 30 min—Kinetex) to yield 6.44 mg of the pure title compound (7.4%). HPLC t_{R}=3.56 min. LC/TOF-MS: exact mass 1737.842 (calculated 1737.846). C₇₅H₁₂₄ClN₂₁O₂₀S₂ (MW=1739.504).

### Example 16: Synthesis XV: Synthesis of Compound Ac-Aib-Pcf-[Cys-Lys-Gln(Gu)-Aib-Asn-Cys]-Tle-Thr-Cmp-lys(DOTA)-NH₂ (PSM-0494)

The linear sequence of the peptide (Ac-Aib-Pcf-Cys-Lys-Glu(OAll)-Aib-Asn-Cys-Tle-Thr-Cmp-lys(Dde)-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. The Dde group was cleaved from the C-terminal lysine side chain and DOTA coupled to the liberated amino function by performing a ‘Dde/ivDde deprotection’ and a ‘Coupling of chelators building blocks’ step, respectively. Prior to the formation of the guanidine moiety on the glutamate side chain by performing the ‘Synthesis of acyl guanidine side chains’ method, the allyl ester protection of the latter was removed under the conditions of an ‘Alloc/Allyl deprotection’. The linear peptide (Ac-Aib-Pcf-Cys-Lys-Glu(Gu)-Aib-Asn-Cys-Tle-Thr-Cmp-lys(DOTA)-NH₂) was cleaved from the solid support employing by employing ‘Cleavage method C: Cleavage of unprotected fragments (complete resin cleavage)’ and the obtained crude material subjected to ‘Cyclization method A: Disulfide cyclization’. After lyophilisation of the reaction solution the crude product was purified by ‘Preparative HPLC’ (10 to 30% B in 30 min—Kinetex) to yield 16.83 mg of the pure title compound (17.9%). HPLC t_{R}=4.73 min. LC/TOF-MS: exact mass 1879.878 (calculated 1879.884). C₈₀H₁₃₀ClN₂₃O₂₃S₂ (MW=1881.617).

### Example 17: Synthesis XVI: Synthesis of Compound Ac-Aib-Pcf-[Cys-Lys-Arg(Ac)-Aib-Asn-Cys]-Tle-Thr-Cmp-lys(DOTA)-NH₂ (PSM-0298)

The linear sequence of the peptide (Ac-Aib-Pcf-Cys-Lys-Orn(Dde)-Aib-Asn-Cys-Tle-Thr-Cmp-lys(Alloc)-NH₂) was assembled according to the ‘General procedures for Automated/Semi-automated Solid-Phase Synthesis’ in a 50 mol scale on a DEG Rink amide resin applying the ‘Solid phase peptide synthesis (SPPS)’ method with an N-terminal acetylation as final step. The Dde protecting group was released from the ornithine side chain by a ‘Dde/ivDde deprotection’ and the liberated amine of the latter transformed into an acetylated arginine side chain by preforming the steps of a ‘Synthesis of acetylated guanidin side chains’ procedure. The amino group of the C-terminal D-lysine was freed from the alloc protection by an ‘Alloc/Allyl deprotection’ and DOTA coupled to the latter by a ‘Coupling of chelators building blocks’ step. The peptide (Ac-Aib-Pcf-Cys-Lys-Arg(Ac)-Aib-Asn-Cys-Tle-Thr-Cmp-lys(DOTA)-NH₂) was then cleaved from the solid support, subjected to ‘Cyclization method A: Disulfide cyclization’, lyophilised and purified by ‘Preparative HPLC″ HPLC’ (10 to 30% B in 30 min—Kinetex), which finally yielded 27.37 mg of the pure title compound (28.7%). HPLC t_{R}=4.67 min. LC/TOF-MS: exact mass 1907.915 (calculated 1907.915). C₈₂H₁₃₄ClN₂₃O₂₃S₂ (MW=1909.67).

### Example 18: Synthesis XVII: Synthesis of Compound DOTA-Cmp-Thr-Aib-Pcf-[Cys-Lys-Arg(EtCAyl)-Aib-Asn-Cys]-Tle-Thr-NH₂ (PSM-0579)

The linear sequence of the peptide (DOTA-Cmp-Thr-Aib-Pcf-Cys-Lys-Orn(Alloc)-Aib-Asn-Cys-Tle-Thr-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with coupling of DOTA as final building block. The Alloc protecting group was removed from the ornithine side chain by an ‘Alloc/Allyl deprotection’ and the liberated amine used as starting point to transform the ornithine into an ethyl carbamoyl arginine by performing the steps of the ‘Synthesis of carbamoylated guanidine side chains I’ procedure. The linear peptide was cleaved from the solid support employing ‘Cleavage method C: Cleavage of unprotected fragments (complete resin cleavage)’ and the obtained crude material subjected to ‘Cyclization method A: Disulfide cyclization’. After lyophilisation of the reaction solution the crude product was purified by ‘Preparative HPLC’ (10 to 30% B in 30 min—Kinetex) to yield 7.75 mg of the pure title compound (8.3%). HPLC t_{R}=4.4 min. LC/TOF-MS: exact mass 1867.881 (calculated 1867.884). C₇₉H₁₃₀ClN₂₃O₂₃S₂ (MW=1869.606).

### Example 19: Synthesis XVIII: Synthesis of Compound DOTA-Cmp-Thr-Aib-Pcf-[Cys-Lys-Urr-Aib-Asn-Cys]-Tle-Thr-NH₂ (PSM-0546)

The linear sequence of the peptide (DOTA-Cmp-Thr-Aib-Pcf-Cys-Lys-Dap(ivDde)-Aib-Asn-Cys]-Tle-Thr-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with coupling of DOTA as final building block. The Dde protecting group was removed from the diamine propionic acid side chain (Dap) by a ‘Dde/ivDde deprotection’ and the liberated amine used as starting point of transform the Dap residue into 3-keto-4-aza-arginine (Urr) by performing the steps of the ‘Synthesis of carbamoylated guanidine side chains II’ procedure. The linear peptide was cleaved from the solid support employing ‘Cleavage method C: Cleavage of unprotected fragments (complete resin cleavage)’ and the obtained crude material subjected to ‘Cyclization method A: Disulfide cyclization’. After lyophilisation of the reaction solution the crude product was purified by ‘Preparative HPLC’ (10 to 30% B in 30 min—Kinetex) to yield 11.5 mg of the pure title compound (12.7%). HPLC t_{R}=4.09 min. LC/TOF-MS: exact mass 1811.824 (calculated 1811.821). C₇₅H₁₂₂ClN₂₃O₂₃S₂ (MW=1813.5).

### Example 20: Synthesis XIX: Synthesis of Compound Ac-Thr-Aib-Phe-[Smc-Lys-Arg-Ala-Asn-Cys]-Tle-Thr-Asp-NH₂ (Alternative: Ac-Thr-Aib-Phe-[Cys-Lys-Arg-Ala-Asn-Smc]-Tle-Thr-Asp-NH₂) (PSM-0567)

The linear sequence of the peptide (Ac-Thr-Aib-Phe-Cys-Lys-Arg-Ala-Asn-Cys-Tle-Thr-Asp-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’ method with an N-terminal acetylation as final step. After cleavage of the linear peptide from the synthesis resin employing ‘Cleavage method C: Cleavage of unprotected fragments (complete resin cleavage)’ (cleavage time 2 hours) the obtained crude material was subjected to ‘Cyclization method B: Cyclization with diodo methane’. After lyophilisation of the reaction solution the crude product was purified by ‘Preparative HPLC’ (10 to 30% B in 30 min—Kinetex) to yield 5.37 mg of the pure title compound (7.6%). HPLC t_{R}=3.99 min. LC/TOF-MS: exact mass 1408.646 (calculated 1408.659). C₅₉H₉₆N₁₈O₁₈S₂ (MW=1409.638).

### Example 21: Synthesis XXa: Synthesis of Compound InDOTA-Ttds-Thr-Aib-Phe-[Cys-Lys-Arg-Ala-Asn-Cys]-Tle-Thr-Asp-NH₂ (PSM-0277)

The peptide DOTA-Ttds-Thr-Aib-Phe-[Cys-Lys-Arg-Ala-Asn-Cys]-Tle-Thr-Asp-NH₂ (PSM-0193, 1.96 μmol) and InCl₃·4H₂O (5.88 μmol, 3 eq.) were dissolved in 0.4 M sodium acetate buffer (pH=5, 3 mL) and heated to 50° C. for 20 minutes (in case of a DOTAM complex 2 hours). Afterwards the reaction solution was directed to purification by ‘Solid phase extraction (SPE)’ to yield 2.84 mg of the pure title compound (67.3%). HPLC t_{R}=4.46 min. LC/TOF-MS: exact mass 2150.972 (calculated 2150.878). C₈₆H₁₄₁InN₂₄O₂₉S₂ (MW=2154.137).

### Example 22: Synthesis XXb: Synthesis of Compound LuDOTA-Cmp-Thr-Aib-Phe-[Cys-Lys-Arg-Aib-Asn-Cys]-Tle-Thr-NH₂ (PSM-0213)

The peptide DOTA-Cmp-Thr-Aib-Phe-[Cys-Lys-Arg-Aib-Asn-Cys]-Tle-Thr-NH₂ (PSM-0492, 2.84 μmol) and LuCl₃ (8.52 μmol, 3 eq.) were dissolved in 0.2 M sodium acetate buffer (pH=5, 3 mL) and heated to 50° C. for 20 minutes. Afterwards the reaction solution was directed to purification by ‘Solid phase extraction (SPE)’ to yield 3.68 mg of the pure title compound (66.9%). HPLC t_{R}=4.44 min. LC/TOF-MS: exact mass 1934.817 (calculated 1934.803). C₇₆H₁₂₃LuN₂₂O₂₂S₂ (MW=1936.026).

### Example 23: Synthesis XXc: Synthesis of Compound GaDOTA-Ttds-Thr-Aib-Phe-[Cys-Lys-Arg-Ala-Asn-Cys]-Tle-Thr-NH₂ (PSM-0196)

The peptide DOTA-Ttds-Thr-Aib-Phe-[Cys-Lys-Arg-Ala-Asn-Cys]-Tle-Thr-NH₂ (PSM-0237, 5.2 μmol) and Ga(NO₃)₃·H₂O (15.6 μmol, 3 eq.) were dissolved in 0.2 M sodium acetate buffer (pH=5, 3 mL) and heated to 50° C. for 20 minutes. Afterwards the reaction solution was directed to purification by ‘Solid phase extraction (SPE)’ to yield 8.92 mg of the pure title compound (86%). HPLC t_{R}=4.02 min. LC/TOF-MS: exact mass 1991.87 (calculated 1991.872). C₈₂H₁₃₆GaN₂₃O₂₆S₂ (MW=1993.954).

### Example 24: Synthesis XXd: Synthesis of Compound EuDOTA-Cmp-Thr-Aib-Pcf-[Cys-Lys-Arg-Aib-Asn-Cys]-Tle-Thr-NH₂ (PSM-0468)

The peptide DOTA-Cmp-Thr-Aib-Pcf-[Cys-Lys-Arg-Aib-Asn-Cys]-Tle-Thr-NH₂ (PSM-0445, 2.77 μmol) and EuCl₃·6H₂O (8.3 μmol, 3 eq.) were dissolved in 0.1 M ammonium acetate buffer (pH=8, 2 mL) and the resulting solution stirred overnight at room temperature. Afterwards, the reaction solution was directed to purification by ‘Solid phase extraction (SPE)’ to yield 3.89 mg of the pure title compound (72.1%). HPLC t_{R}=11.88 min. LC/TOF-MS: exact mass 1944.845 (calculated 1944.743). C₇₆H₁₂₂ClEuN₂₂O₂₂S₂ (MW=1947.469).

### Example 25: Synthesis XXe: BiDOTA-Cmp-Thr-Aib-Pcf-[Cys-Lys(Me)-Arg(Me)-Aib-Asn-Cys]-Tle-Thr-NH₂ (PSM-0595)

The peptide DOTA-Cmp-Thr-Aib-Pcf-[Cys-Lys(Me)-Arg(Me)-Aib-Asn-Cys]-Tle-Thr-NH₂ (PSM-0194, 3.2 μmol) and Bi(NO₃)₃·5H₂O (9.5 μmol, 3 eq.) were dissolved in 0.4M sodium acetate buffer (pH=4, 3 mL) and the resulting solution stirred at 50° C. for 30 minutes. Afterwards, the reaction solution was directed to purification by ‘Solid phase extraction (SPE)’ to yield 5.33 mg of the pure title compound (81.88%). HPLC t_{R}=5.06 min. LC/TOF-MS: exact mass 2030.835 (calculated 2030.835). C₇₈H₁₂₆BiClN₂₂O₂₂S₂ (MW=2032.538).

### Example 26: Synthesis XXf: LaDOTA-Cmp-Thr-Aib-Pcf-[Cys-Lys(Me)-Arg(Me)-Aib-Asn-Cys]-Tle-Thr-NH₂ (PSM-0596)

The peptide DOTA-Cmp-Thr-Aib-Pcf-[Cys-Lys(Me)-Arg(Me)-Aib-Asn-Cys]-Tle-Thr-NH₂ (PSM-0194, 3.1 μmol) and LaCl₃ were dissolved in 0.2 M sodium phosphate buffer (pH=5, 3 mL) and the resulting solution stirred at 50° C. for 30 minutes. Afterwards, the reaction solution was directed to purification by ‘Solid phase extraction (SPE)’ to yield 3.38 mg of the pure title compound (56.08%). HPLC t_{R}=4.98 min. LC/TOF-MS: exact mass 1959.761 (calculated 1959.762). C₇₈H₁₂₆ClLaN₂₂O₂₂S₂ (MW=1962.463).

### Example 27: Synthesis XXg: PbDOTA-Cmp-Thr-Aib-Pcf-[Cys-Lys(Me)-Arg(Me)-Aib-Asn-Cys]-Tle-Thr-NH₂ (PSM-0597)

The peptide DOTA-Cmp-Thr-Aib-Pcf-[Cys-Lys(Me)-Arg(Me)-Aib-Asn-Cys]-Tle-Thr-NH₂ (PSM-0194, 2.8 μmol) and Pb(CH₃COO)₂·3H₂O (7.5 μmol, 3 eq.) were dissolved in 0.4M ammonium acetate buffer (pH=4, 3 mL) and the resulting solution stirred at 50° C. for 30 minutes. Afterwards, the reaction solution was directed to purification by ‘Solid phase extraction (SPE)’ to yield 4.38 mg of the pure title compound (78.44%). HPLC t_{R}=4.99 min. LC/TOF-MS: exact mass 2026.834 (calculated 2026.836). C₇₈H₁₂₇ClN₂₂O₂₂PbS₂ (MW=2031.782).

### Example 28: Synthesis XXI: DOTA-Cmp-Thr-Aib-Pcf-[Cys-Lys(Me)-RMe2a-Aib-Asn-Cys]-Tle-Thr-NH₂ (PSM-0590)

The linear sequence of the peptide (Alloc-Om-Aib-Asn-Cys-Tle-Thr-NH₂) was assembled according to the ‘General procedures for Automated/Semi-automated Solid-Phase Synthesis’ in a 50 μmol scale on a DEG Rink amide resin applying the ‘Solid phase peptide synthesis (SPPS)’. The δ-amino function of the ornithine building block was methylated by implementing the ‘Selective N-Methylation under Mitsunobu conditions’ procedure. The specifically methylated arginine side chain was created from the methylated ornithine by subjecting the peptide resin to the ‘Synthesis of alkylated guanidine side chains’ reaction sequences. After the N-terminal Alloc protection was removed from the intermediate peptide resin (Alloc-RMe2a-Aib-Asn-Cys-Tle-Thr-NH₂) by employing an ‘Alloc/Allyl deprotection’, the remaining peptide sequence was assembled according to the ‘General procedures for Automated/Semi-automated Solid-Phase Synthesis’ with coupling of DOTA as final building block. The resin was treated under the conditions of the ‘Cleavage method C: Cleavage of unprotected fragments (complete resin cleavage)’ and the obtained crude material was subjected to cyclization using the method ‘Cyclization method A: Disulfide cyclization’. After lyophilisation of the reaction solution the crude product was purified by ‘Preparative HPLC’ (10 to 30% B in 30 min—Kinetex) to yield 8.99 mg of the pure title compound (9.8%). HPLC t_{R}=3.47 min. LC/TOF-MS: exact mass 1839.002 (calculated 1838.894). C₇₉H₁₃₁ClN₂₂O₂₂S₂ (MW=1840.608).

### Example 29: Synthesis XXII: Macropa-Cmp-Thr-Aib-Pcf-[Cys-Lys(Me)-Arg(Me)-Aib-Asn-Cys]-Tle-Thr-NH₂ (PSM-0515)

The linear sequence of the peptide (H-Cmp-Thr(tBu)-Aib-Pcf-Cys(SDmp)-Lys(Me,Boc)-Arg(Me,Pbf)-Aib-Asn(Trt)-Cys(SDmp)-Tle-Thr(tBu)-NH₂) was assembled according to the ‘General procedures for Automated/Semi-automated Solid-Phase Synthesis’ in a 100 μmol scale on a Sieber amide resin applying the ‘Solid phase peptide synthesis (SPPS)’ method. The SDmp protecting groups were removed from the cysteines by employing the ‘SDmp deprotection’ deprotection method and the partially protected peptide fragment detached from the resin employing ‘Cleavage method B: Cleavage of protected fragments from hyper-acid labile resin’ method. The obtained crude material was subjected to ‘Cyclization method A: Disulfide cyclization’. After lyophilisation of the reaction solution the crude product was purified by ‘Preparative HPLC’ (55 to 90% B in 30 min—Kinetex) to yield 49.66 mg of the cyclic partially protected peptide H-Cmp-Thr(tBu)-Aib-Pcf-[Cys-Lys(Me,Boc)-Arg(Me,Pbf)-Aib-Asn(Trt)-Cys]-Tle-Thr(tBu)-NH₂. To the solution of 35 mg (16.3 μmol) in DMF (0.5 mL), the Macropa-chelator building block (carboxylic acid functions at the pyridine rings protected as ethyl esters) (16.4 mg, 22.8 μmol, 1.4 eq.), HATU (8.7 mg, 22.8 μmol, 1.4 eq.) and DIPEA (7.9 μL, 45.6 μmol, 2.8 eq) were added. After the mixture was stirred for 2 hours, the volatiles were removed in the vacuum. The remainder was dissolved in MeOH (2 mL) and the ethyl ester groups were removed by addition of 0.1 M NaOH solution, which was carefully analyzed by LC-MS. Afterward the solvent was removed in the vacuum and the remainder subjected to lyophilisation. The obtained crude material was purified by ‘Preparative HPLC’ (15 to 35% B in 30 min—Kinetex) to yield 1.62 mg of the pure title compound (1.1% overall yield). HPLC t_{R}=4.21 min. LC/TOF-MS: exact mass 2081.973 (calculated 2081.983). C₉₃H₁₄₄ClN₂₃O₂₅S₂ (MW=2083.866).

### Example 30: Synthesized Compounds Summary

Characterization data (HPLC/MS) for the compounds shown below are included in Table 6, following Example 29. Reference to the synthetic strategy used to prepare each compound is also included in Table 6.

The following compounds were synthesized:
compound Ac-Thr-Aib-Phe-[Cys-Lys-Arg-Aib-Asn-Cys]-Tle-Thr-NH₂ (PSM-0178) of the following formula

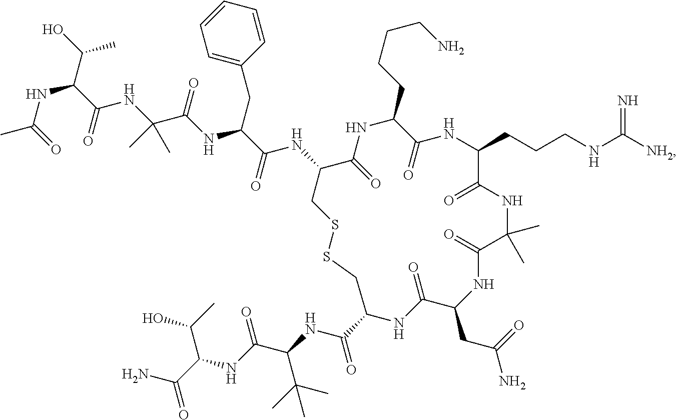 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Pcf-[Cys-Lys-Cit-Aib-Asn-Cys]-Tle-Thr-NH₂ (PSM-0179) of the following formula

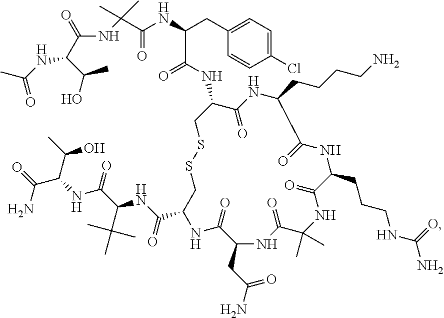 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Phe-[Cys-Lys-Gln-Ala-Asn-Cys]-Tle-Thr-Asp-NH₂ (PSM-0180) of the following formula

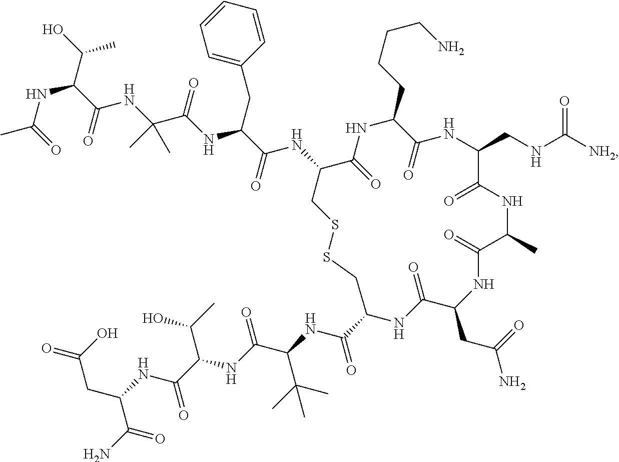 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CNC(N)=O)NC(=O)[C@H](CCCCN)NC1=O)[C@@H](C)O]

compound Ac-Thr-Aib-Phe-[Cys-Lys-Arg-Hyp-Asn-Cys]-Tle-Thr-Asp-NH₂ (PSM-0181) of the following formula

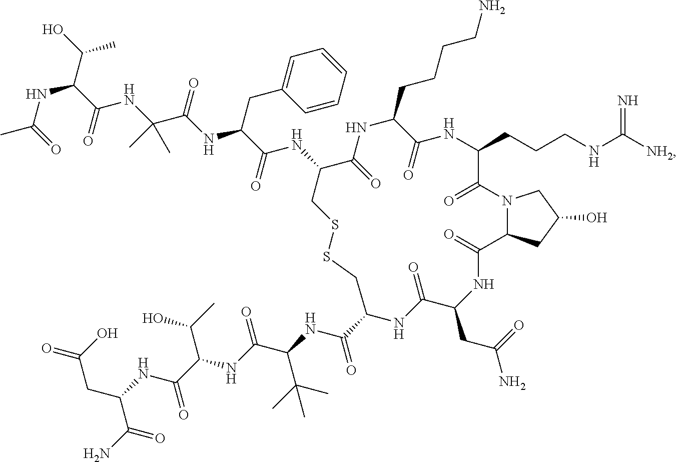 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@@H]2C[C@@H](O)CN2C(=O)[C@H](CCCNC(=N)N)NC(=O)[C@H](CCCCN)NC1=O)[C@@H](C)O]

compound Ac-Thr-Ala-Phe-[Cys-Lys-Arg-Ala-Asn-Cys]-Nle-thr-Asp-NH₂ (PSM-0182) of the following formula

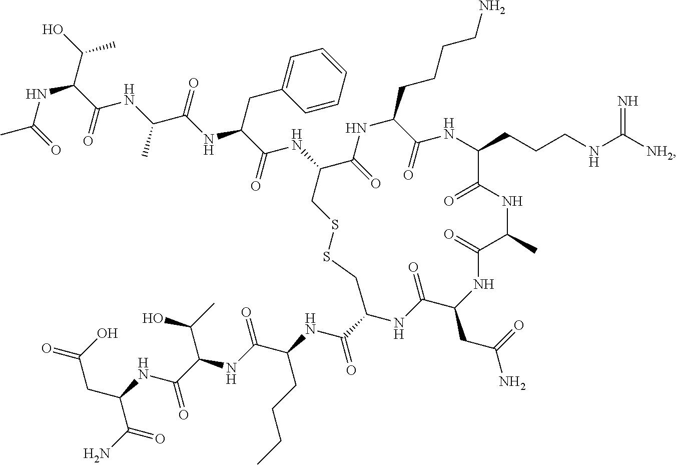 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@@H](C(=O)N[C@H](CC(=O)O)C(N)=O)[C@H](C)O]

compound Ac-Thr-Aib-Phe-[Cys-Lys-Arg-Aib-Asn-Cys]-Tle-Thr-Asp-Ttds-Ttds-AF488N3K-NH₂ (PSM-0183) of the following formula

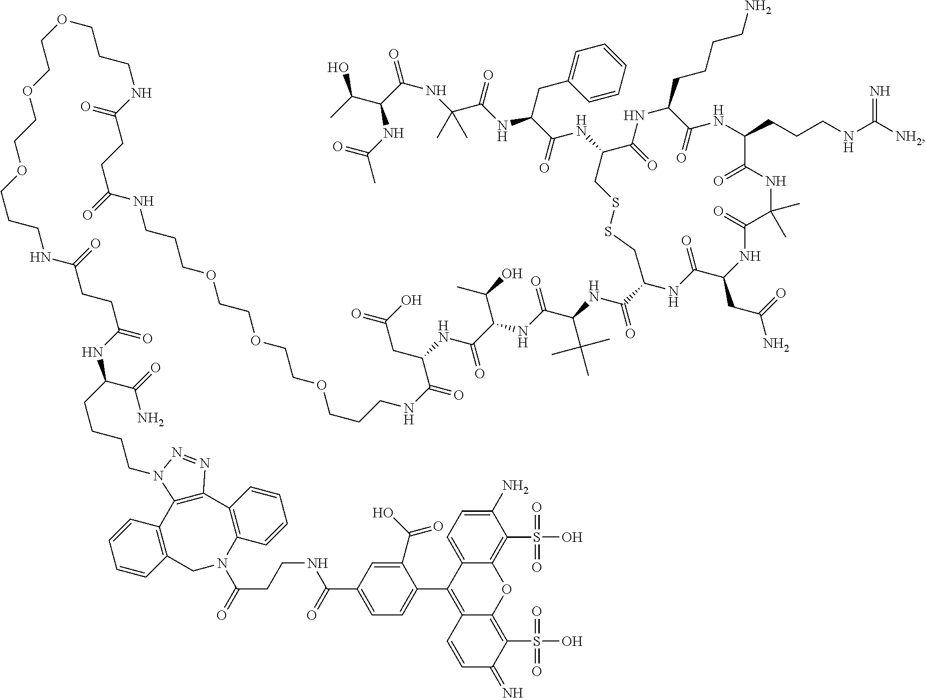 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)NCCCOCCOCCOCCCNC(=O)CCC(=O)N[C@H](CCCCn2nnc3c2-c2ccccc2CN(C(=O)CCNC(=O)c2ccc(-c4c5ccc(=N)c(S(=O)(=O)O)c-5oc5c(S(=O)(=O)O)c(N)ccc45)c(C(=O)O)c2)c2ccccc2-3)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Thr-Aib-Phe-[Cys-Lys-Arg-Aib-Asn-Cys]-Tle-NH₂ (PSM-0184) of the following formula

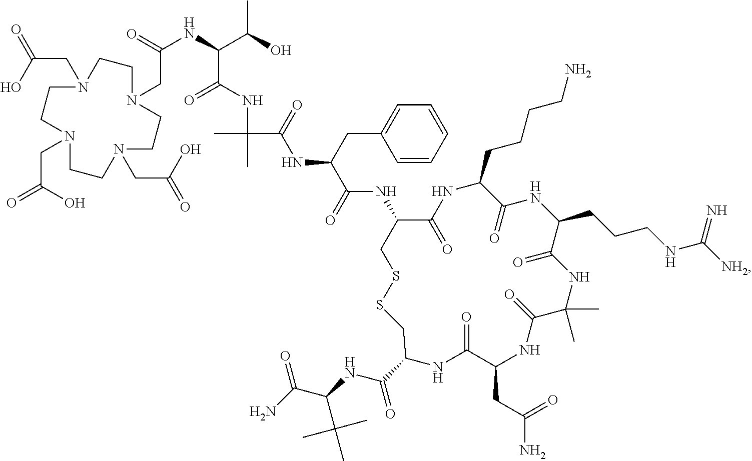 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccccc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound LuDOTA-Cmp-Thr-Aib-Pcf-[Cys-Lys(Me)-Arg(Me)-Aib-Asn-Cys]-Tle-NH₂ (PSM-0185) of the following formula

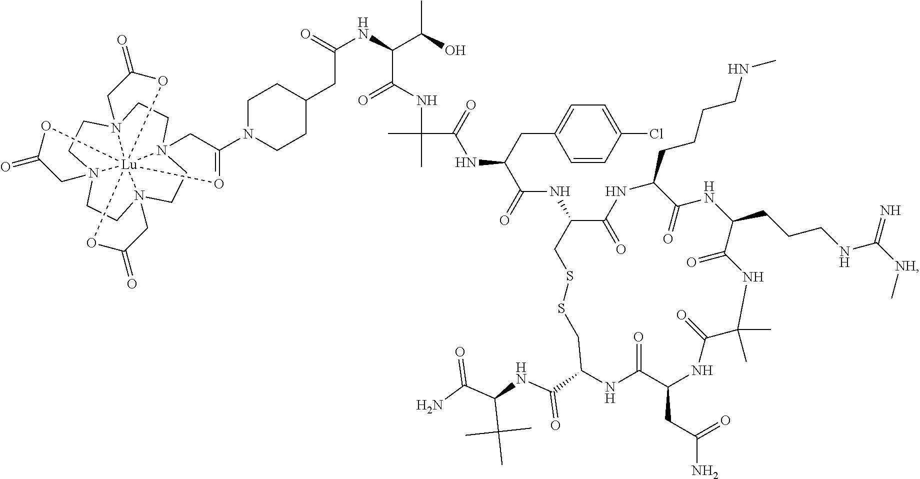

compound Ac-Pamp-Aib-Pcf-[Cys-Lys-Arg-Aib-Asn-Cys]-Tle-en-DOTA (PSM-0186) of the following formula

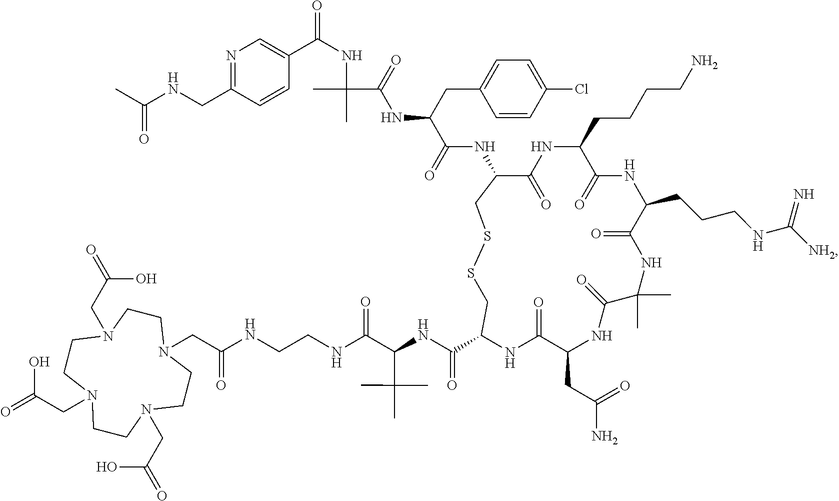 [SMILES: CC(=O)NCc1ccc(C(=O)NC(C)(C)C(=O)N[C@@H](Cc2ccc(Cl)cc2)C(=O)N[C@H]2CSSC[C@@H](C(=O)N[C@H](C(=O)NCCNC(=O)CN3CCN(CC(=O)O)CCN(CC(=O)O)CCN(CC(=O)O)CC3)C(C)(C)C)NC(=O)[C@H](CC(N)=O)NC(=O)C(C)(C)NC(=O)[C@H](CCCNC(=N)N)NC(=O)[C@H](CCCCN)NC2=O)cn1]

compound Ac-Thr-Deg-Phe-[Cys-Lys-Arg-Aib-Asn-Cys]-Tle-Thr-NH₂ (PSM-0187) of the following formula

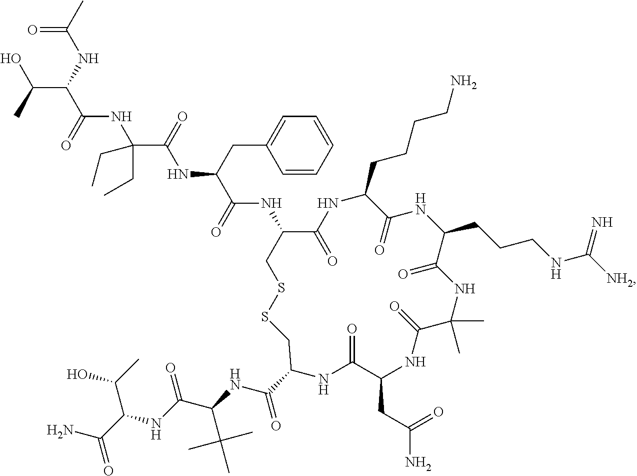 [SMILES: CCC(CC)(NC(=O)[C@@H](NC(C)=O)[C@@H](C)O)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound SaPr-Aib-Pcf-[Cys-Lys-Arg-Lys(DOTA-APAc)-Asn-Cys]-Tle-Thr-NH₂ (PSM-0188) of the following formula

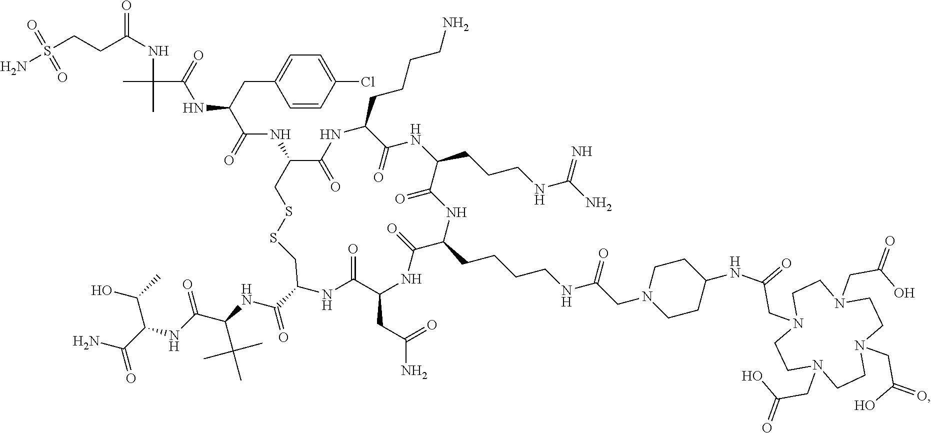 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCS(N)(=O)=O)C(=O)N[C@@H](CCCCN)C(=O)N[C@@H](CCCNC(=N)N)C(=O)N[C@@H](CCCCNC(=O)CN2CCC(NC(=O)CN3CCN(CC(=O)O)CCN(CC(=O)O)CCN(CC(=O)O)CC3)CC2)C(=O)N[C@@H](CC(N)=O)C(=O)N1)C(C)(C)C)C(N)=O]

compound Ac-Thr-Aib-Pcf-[Cys-Lys-Gln(Gu)-Aib-Asn-Cys]-Tle-Thr-NH₂ (PSM-0189) of the following formula

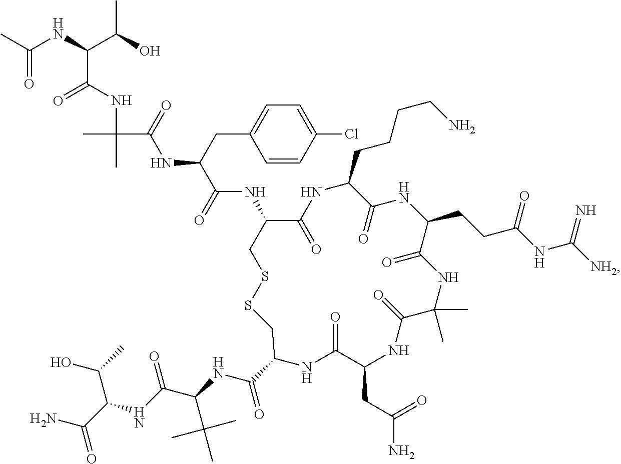 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCC(=O)NC(=N)N)NC(=O)[C@H](CCCCN)NC1=O)[C@@H](C)O]

compound Ac-Thr-Aib-Pcf-[Cys-Lys-Arg-Aib-Asn-Cys]-Tle-Thr-lys(DOTA-Cmp)-NH₂ (PSM-0190) of the following formula

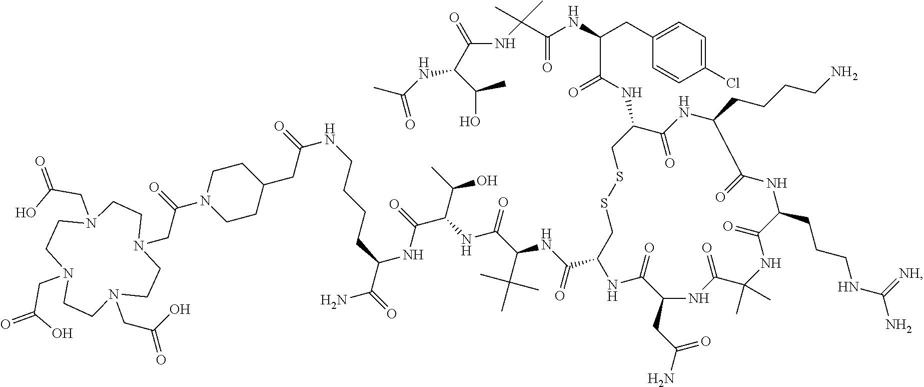 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Thr-APAc-lys(DOTA)-NH₂ (PSM-0191) of the following formula

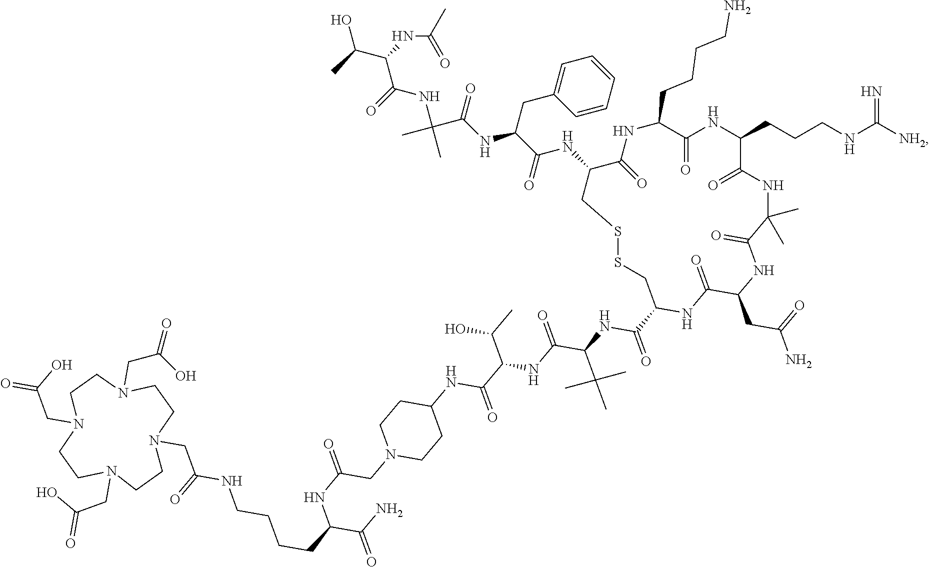 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NC2CCN(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound H-Cmp-Thr-Aib-Miy-[Cys-Lys(Me)-Arg(Me)-Aib-Asn-Cys]-Tle-Thr-NH₂ (PSM-0192) of the following formula

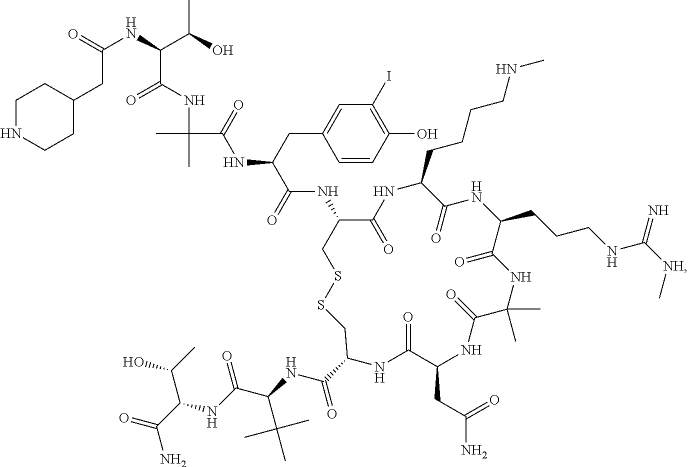 [SMILES: CNCCCC[C@@H]1NC(=O)[C@@H](NC(=O)[C@H](Cc2ccc(O)c(I)c2)NC(=O)C(C)(C)NC(=O)[C@@H](NC(=O)CC2CCNCC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound DOTA-Ttds-Thr-Aib-Phe-[Cys-Lys-Arg-Ala-Asn-Cys]-Tle-Thr-Asp-NH₂ (PSM-0193) of the following formula

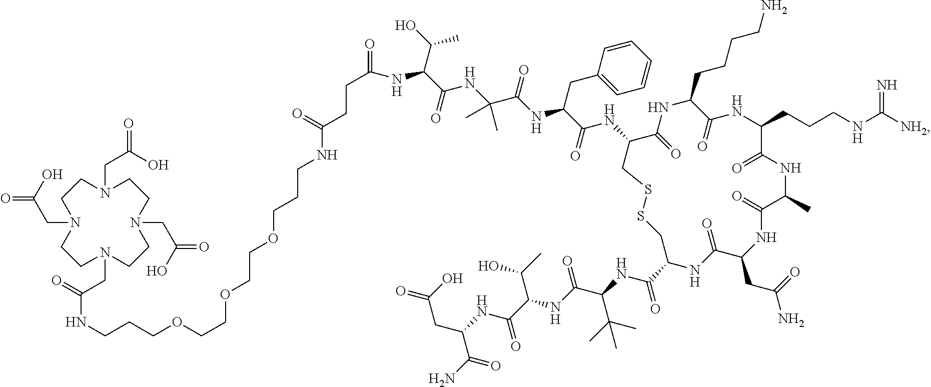 [SMILES: C[C@@H]1NC(=O)[C@H](CCCNC(=N)N)NC(=O)[C@H](CCCCN)NC(=O)[C@@H](NC(=O)[C@H](Cc2ccccc2)NC(=O)C(C)(C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC1=O]

compound DOTA-Cmp-Thr-Aib-Pcf-[Cys-Lys(Me)-Arg(Me)-Aib-Asn-Cys]-Tle-Thr-NH₂ (PSM-0194) of the following formula

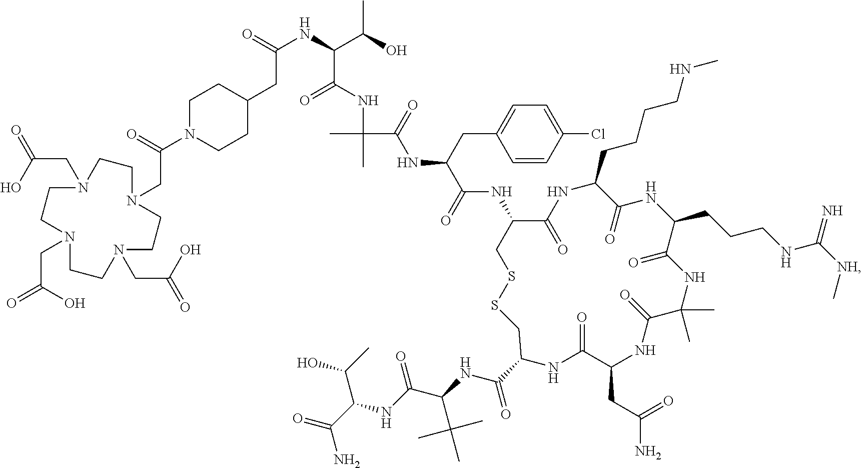 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound InDOTA-Cmp-Thr-Aib-Phe-[Cys-Lys-Arg-Aib-Asn-Cys]-Tle-Thr-NH₂ (PSM-0195) of the following formula

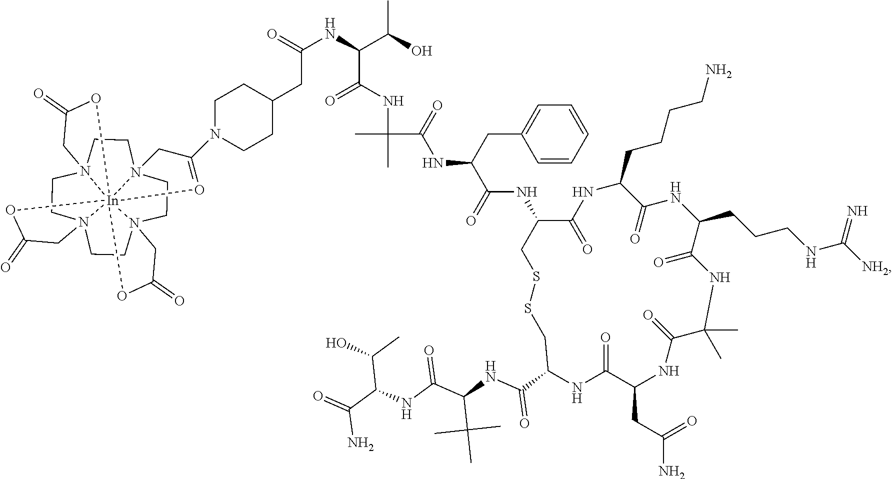

compound GaDOTA-Ttds-Thr-Aib-Phe-[Cys-Lys-Arg-Ala-Asn-Cys]-Tle-Thr-NH₂ (PSM-0196) of the following formula

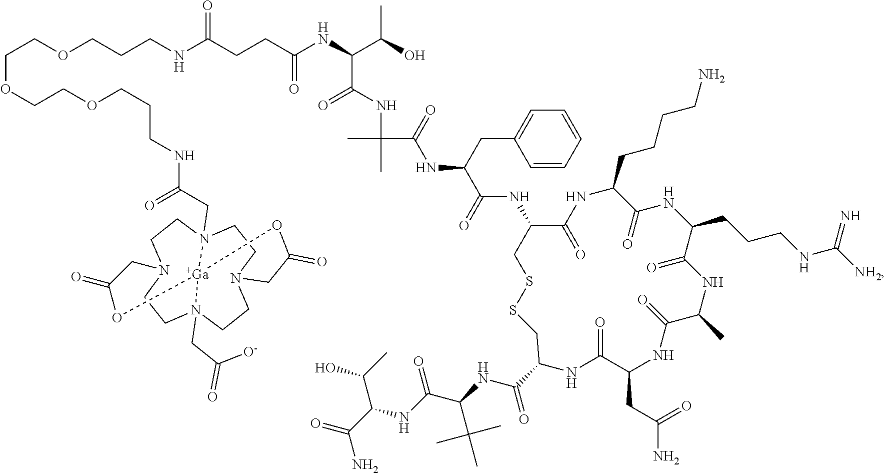

compound Ac-Thr-Ala-Phe-[Cys-Lys-Arg-Glu-Asn-Cys]-Nle-Thr-Asp-NH₂ (PSM-0197) of the following formula

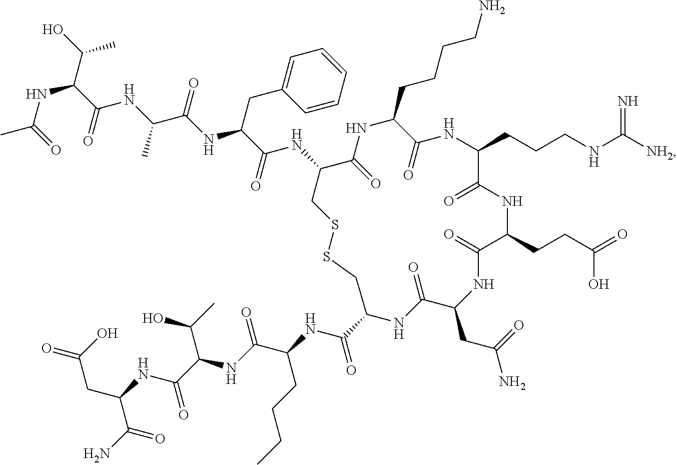 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](CCC(=O)O)C(=O)N[C@@H](CC(N)=O)C(=O)N1)C(=O)N[C@@H](C(=O)N[C@H](CC(=O)O)C(N)=O)[C@H](C)O]

compound DOTA-Cmp-Tle-Aib-Phe-[Cys-Lys-Arg-Aib-Asn-Cys]-Tle-Thr-NH₂ (PSM-0198) of the following formula

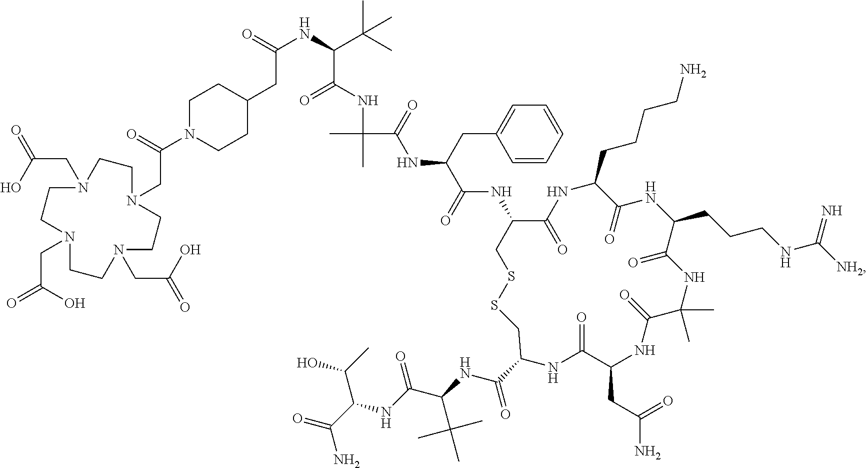 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)C(C)(C)C)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Thr-Aib-Phe-[Cys-Lys-Arg-Aib-Asn-Cys]-Tle-en (PSM-0199) of the following formula

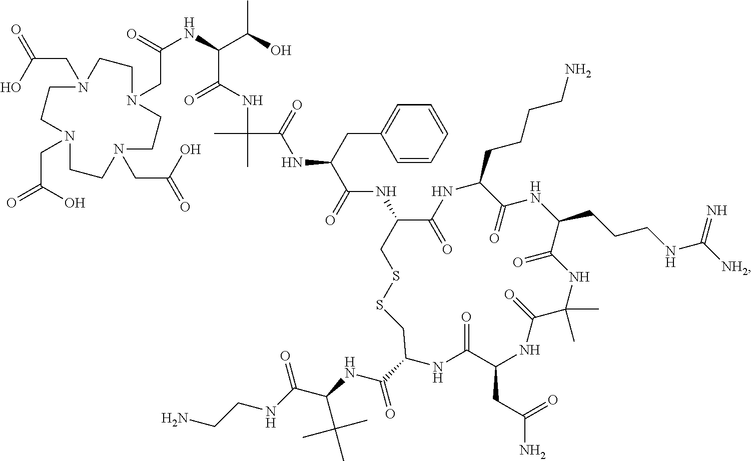 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccccc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Pcf-[Cys-Lys(Me)-Arg-Aib-Asn-Cys]-Tle-NH₂ (PSM-0200) of the following formula

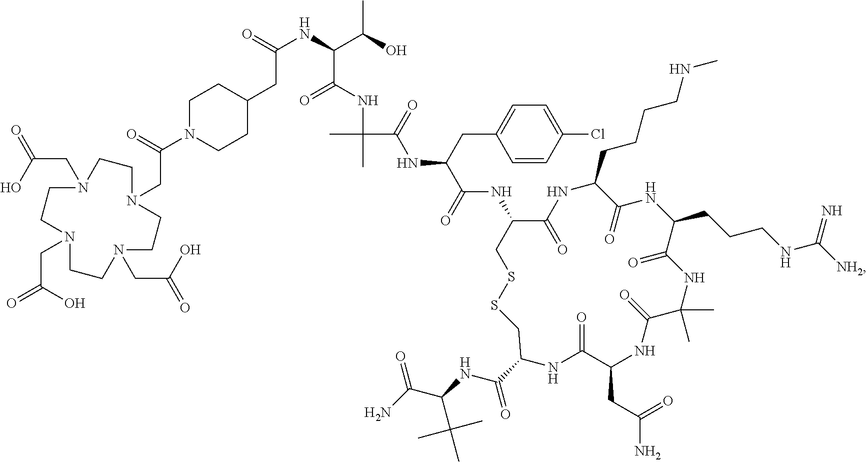 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC1=O]

compound DOTA-Ttds-Thr-Ala-Phe-[Cys-Lys-Arg-Ala-Asn-Cys]-NH₂ (PSM-0201) of the following formula

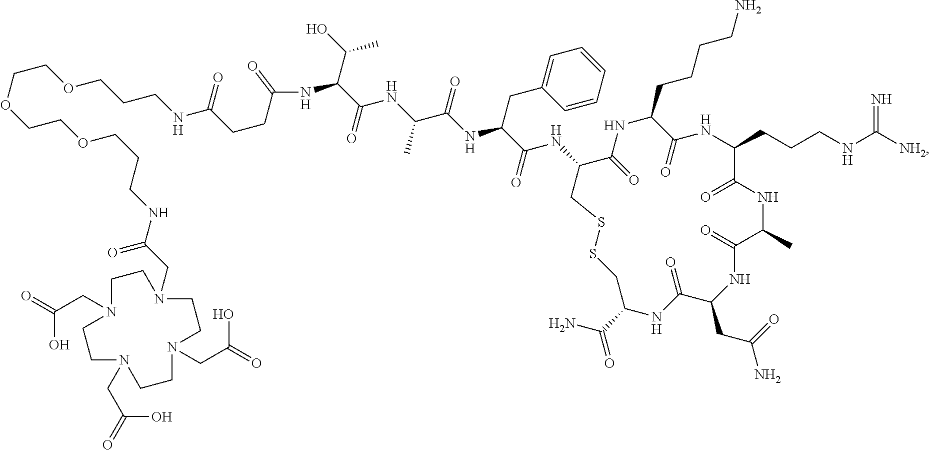 [SMILES: C[C@@H]1NC(=O)[C@H](CCCNC(=N)N)NC(=O)[C@H](CCCCN)NC(=O)[C@@H](NC(=O)[C@H](Cc2ccccc2)NC(=O)[C@H](C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)CSSC[C@@H](C(N)=O)NC(=O)[C@H](CC(N)=O)NC1=O]

compound DOTA-Pamb-Thr-Aib-Phe-[Cys-Lys-Arg-Aib-Asn-Cys]-Tle-Thr-NH₂ (PSM-0202) of the following formula

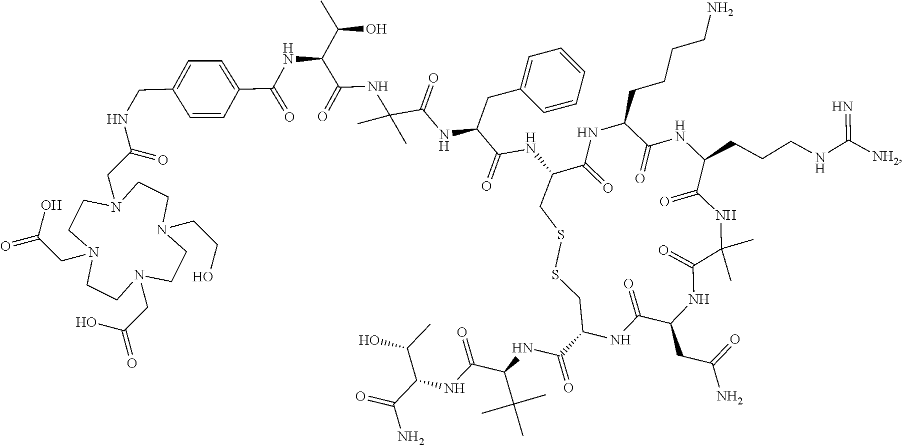 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)c2ccc(CNC(=O)CN3CCN(CCO)CCN(CC(=O)O)CCN(CC(=O)O)CC3)cc2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-KMe2-Arg(Me)-Aib-Asn-Cys]-Tle-Thr-NH₂ (PSM-0203) of the following formula

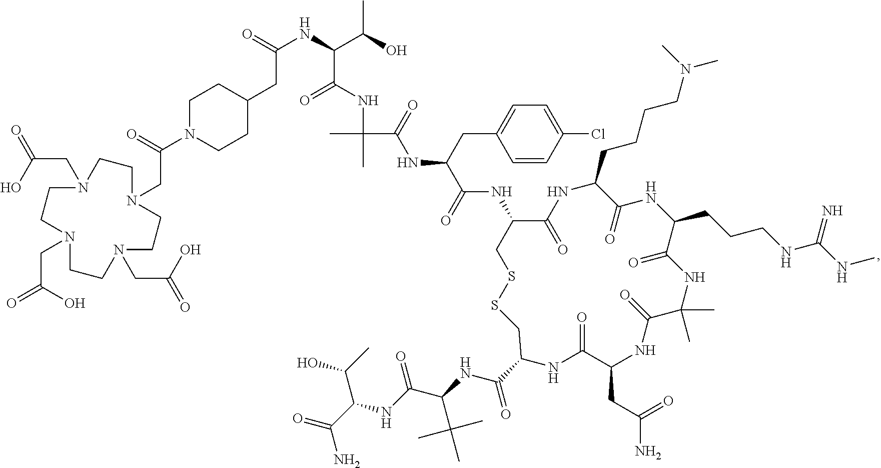 [SMILES: CNC(=N)NCCC[C@@H]1NC(=O)[C@H](CCCCN(C)C)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound nBuCAyl-Thr-Aib-Pcf-[Cys-Lys-Cit-Aib-Asn-Cys]-Tle-Thr-Cmp-lys(DOTA)-NH₂ (PSM-0204) of the following formula

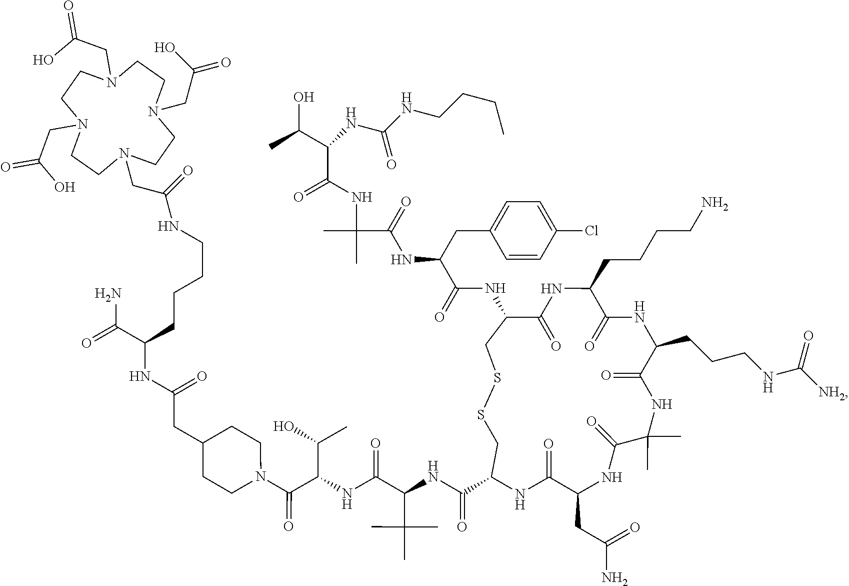 [SMILES: CCCCNC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Pcf-[Cys-Nle-Arg-Aib-Asn-Cys]-Tle-Thr-NH₂ (PSM-0205) of the following formula

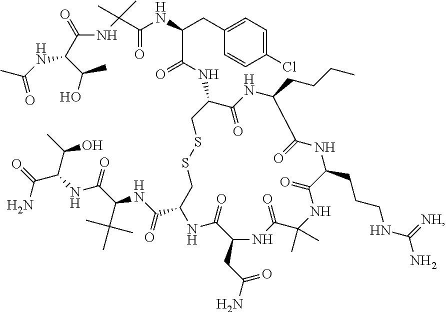 [SMILES: CCCC[C@@H]1NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC1=O]

compound DOTA-Cmp-Thr-Aib-5Brw-[Cys-Lys(Me)-Arg(Me)-Aib-Asn-Cys]-Tle-Thr-NH₂ (PSM-0206) of the following formula

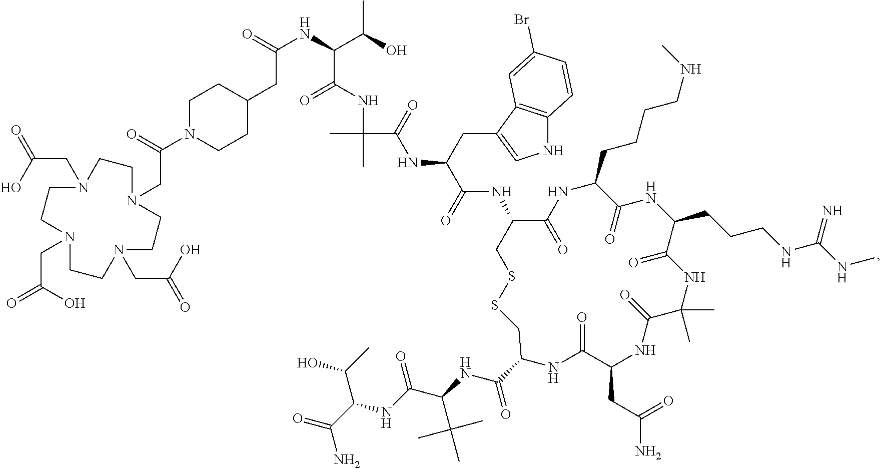 [SMILES: CNCCCC[C@@H]1NC(=O)[C@@H](NC(=O)[C@H](Cc2c[nH]c3ccc(Br)cc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound nBuCAyl-Thr-Aib-Mcf-[Cys-Lys-Cit-Aib-Asn-Cys]-Tle-Thr-Cmp-lys(DOTA)-NH₂ (PSM-0207) of the following formula

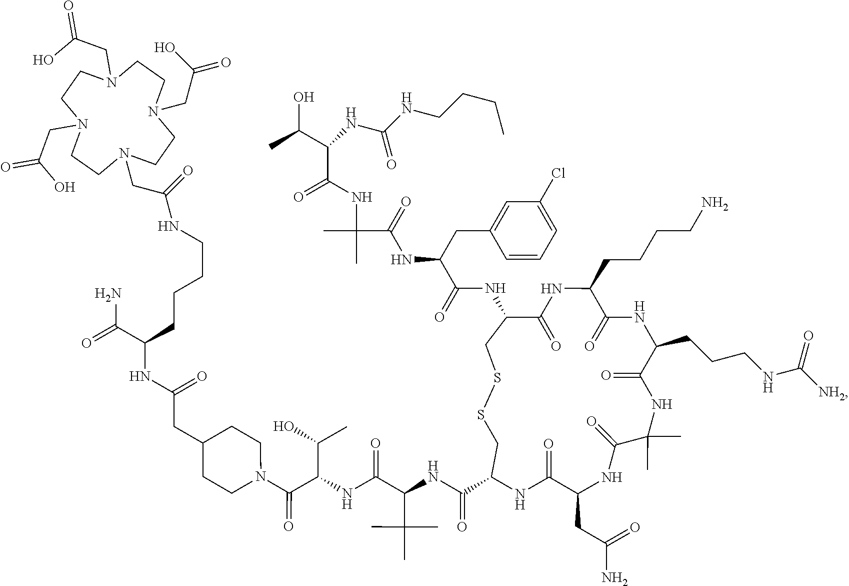 [SMILES: CCCCNC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Phe-[Cys-Lys-Arg-Aib-Asn-Cys]-NHnPen (PSM-0208) of the following formula

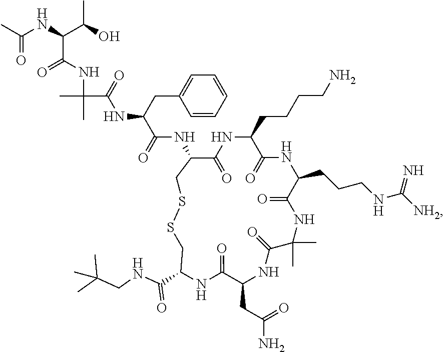 [SMILES: CC(=O)N[C@H](C(=O)NC(C)(C)C(=O)N[C@@H](Cc1ccccc1)C(=O)N[C@H]1CSSC[C@@H](C(=O)NCC(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Bal-Thr-Aib-Phe-[Cys-Lys-Arg-Aib-Asn-Cys]-Tle-Thr-NH₂ (PSM-0209) of the following formula

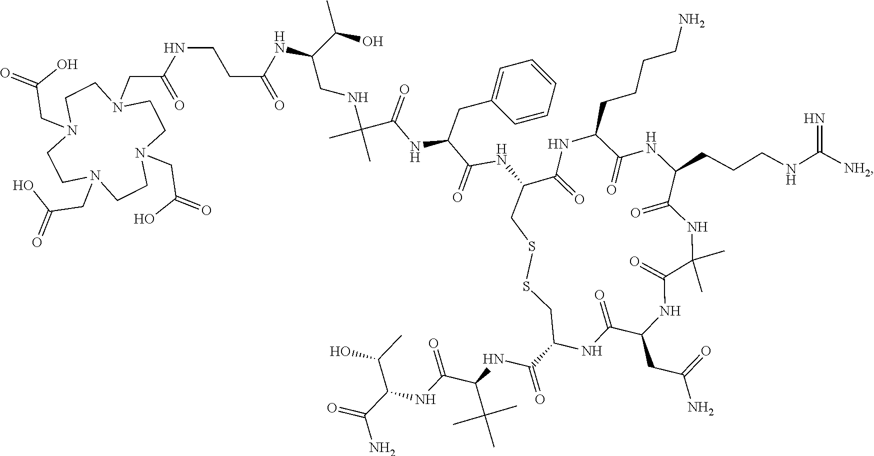 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC[C@@H](NC(=O)CCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Cys-Gln-Arg-Aib-Asn-Cys]-Tle-Thr-Asp-NH₂ (PSM-0210) of the following formula

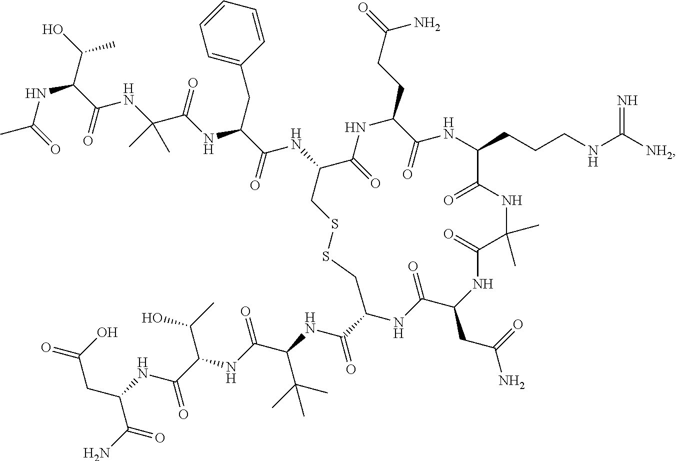 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCC(N)=O)NC1=O)[C@@H](C)O]

compound Ac-Thr-Aib-Phe-[Cys-Lys-Arg-Lys(DOTA-Pab)-Asn-Cys]-Tle-Thr-NH₂ (PSM-0211) of the following formula

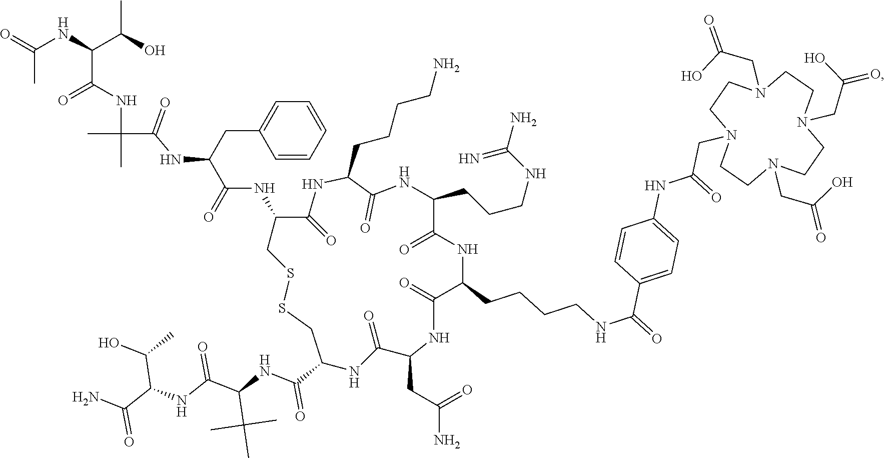 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)c2ccc(NC(=O)CN3CCN(CC(=O)O)CCN(CC(=O)O)CCN(CC(=O)O)CC3)cc2)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Thr-Asp-Ttds-lys(DOTA)-NH₂ (PSM-0212) of the following formula

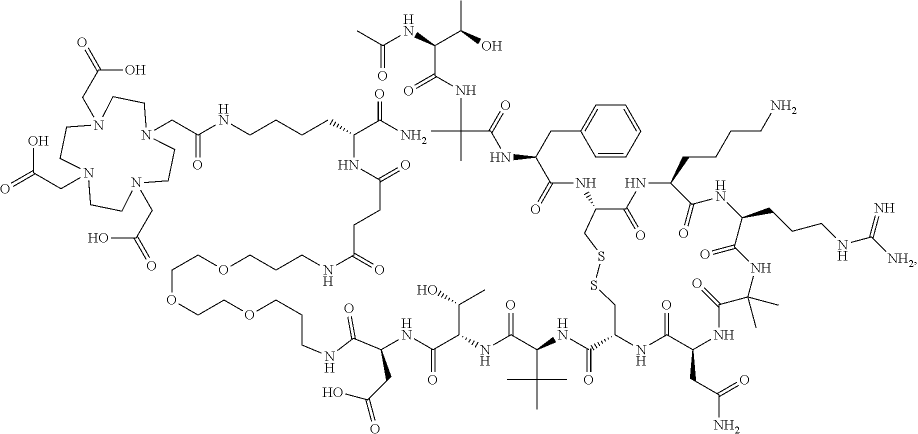 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound LuDOTA-Cmp-Thr-Aib-Phe-[Cys-Lys-Arg-Aib-Asn-Cys]-Tle-Thr-NH₂ (PSM-0213) of the following formula

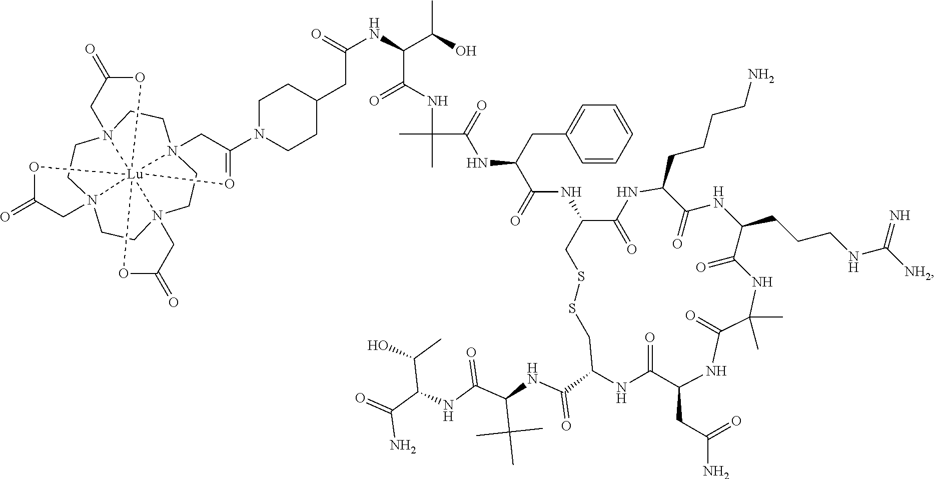

compound nBuCAyl-Thr-Aib-Phe-[Cys-Lys-Arg-Aib-Asn-Cys]-Tle-Thr-Cmp-lys(InDOTA)-NH₂ (PSM-0214) of the following formula

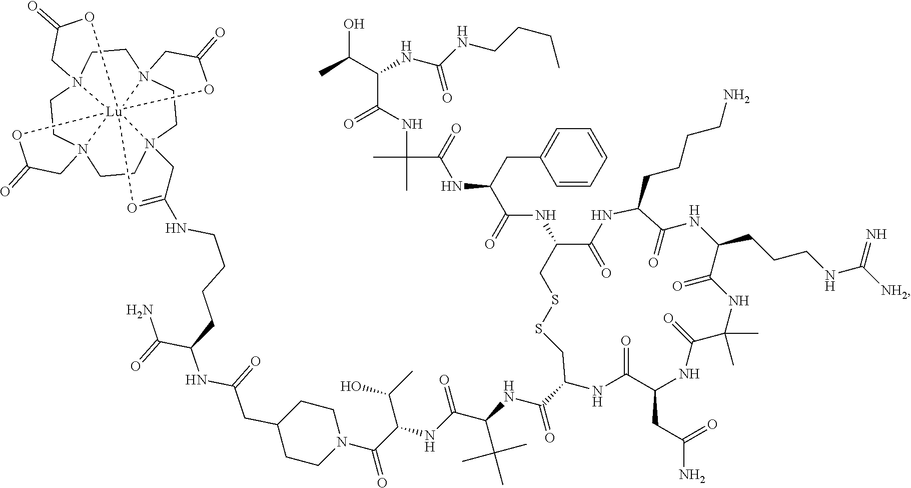

compound SaPr-Aib-Pcf-[Cys-Lys-Arg-Aib-Asn-Cys]-Tle-Thr-Cmp-lys(DOTA)-NH₂ (PSM-0215) of the following formula

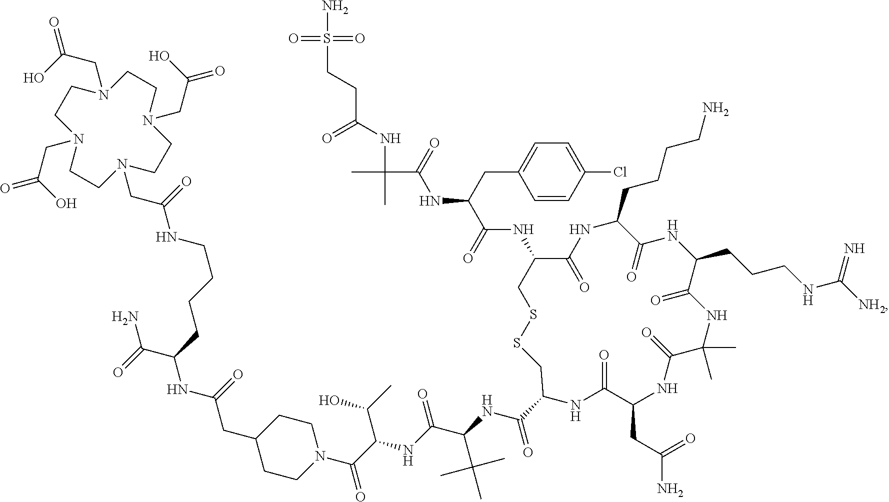 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCS(N)(=O)=O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(=O)N1CCC(CC(=O)N[C@H](CCCCNC(=O)CN2CCN(CC(=O)O)CCN(CC(=O)O)CCN(CC(=O)O)CC2)C(N)=O)CC1]

compound DOTA-Thr-Aib-Pcf-[Cys-Lys-Arg-Aib-Asn-Cys]-Tle-Bal-NH₂ (PSM-0216) of the following formula

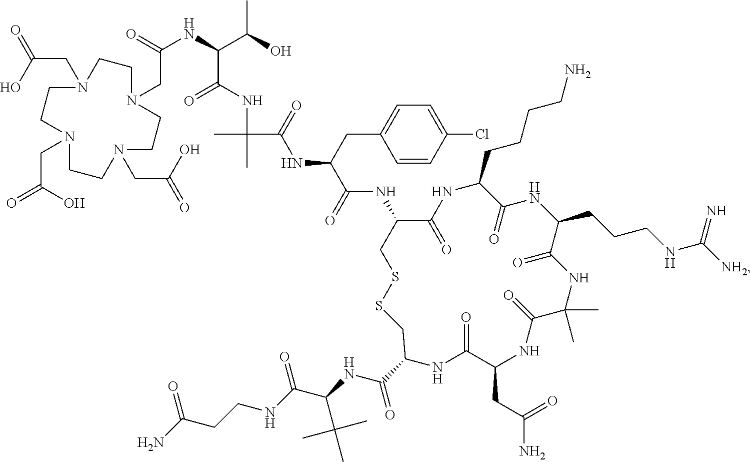 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccc(Cl)cc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Lys-Arg-Dfp-Asn-Cys]-Tle-Thr-Asp-NH₂ (PSM-0217) of the following formula

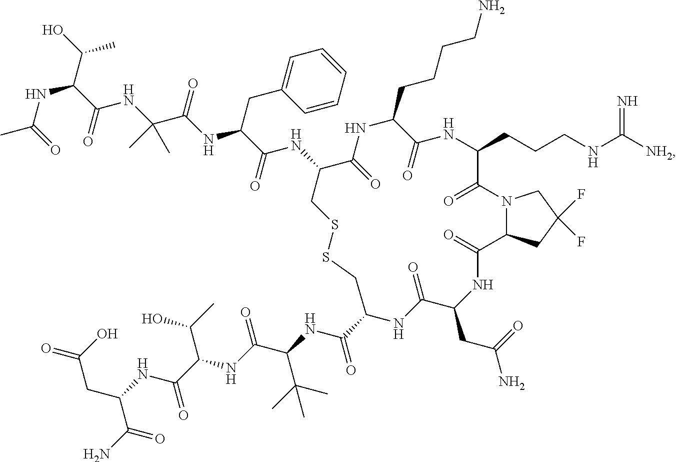 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@@H]2CC(F)(F)CN2C(=O)[C@H](CCCNC(=N)N)NC(=O)[C@H](CCCCN)NC1=O)[C@@H](C)O]

compound DOTAGA-Cmp-Thr-Aib-Phe-[Cys-Lys-Arg-Aib-Asn-Cys]-Tle-Thr-NH₂ (PSM-0218) of the following formula

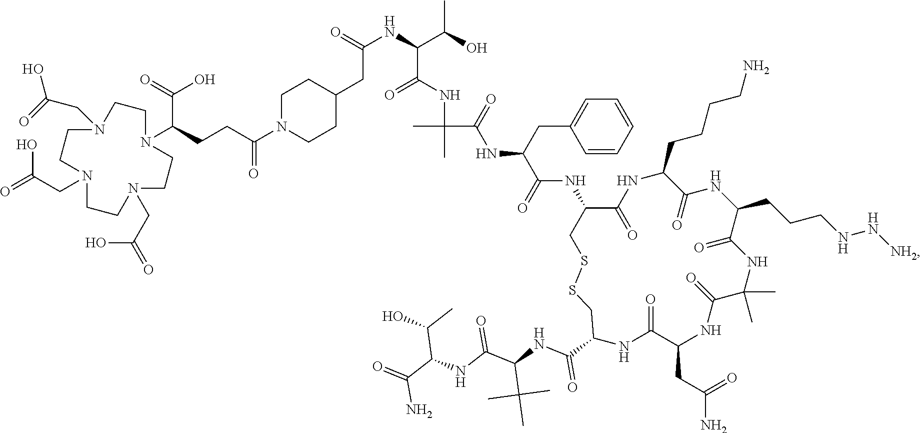 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CC2CCN(C(=O)CC[C@H](C(=O)O)N3CCN(CC(=O)O)CCN(CC(=O)O)CCN(CC(=O)O)CC3)CC2)[C@@H](C)O)C(=O)N[C@@H](CCCCN)C(=O)N[C@@H](CCCNNN)C(=O)NC(C)(C)C(=O)N[C@@H](CC(N)=O)C(=O)N1)C(C)(C)C)C(N)=O]

compound DOTA-Cmp-Leu-Aib-Phe-[Cys-Lys-Arg-Aib-Asn-Cys]-Tle-Thr-NH₂ (PSM-0220) of the following formula

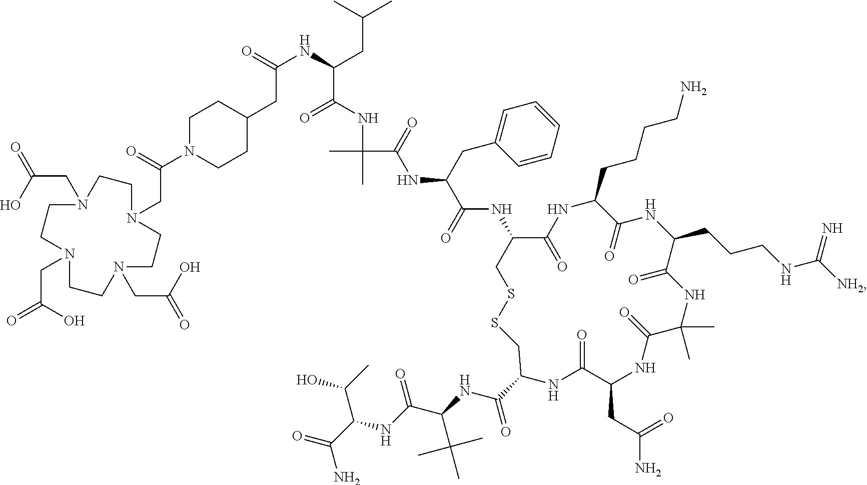 [SMILES: CC(C)C[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Pamb-Aib-Phe-[Cys-Lys-Arg-Aib-Asn-Cys]-Tle-en (PSM-0221) of the following formula

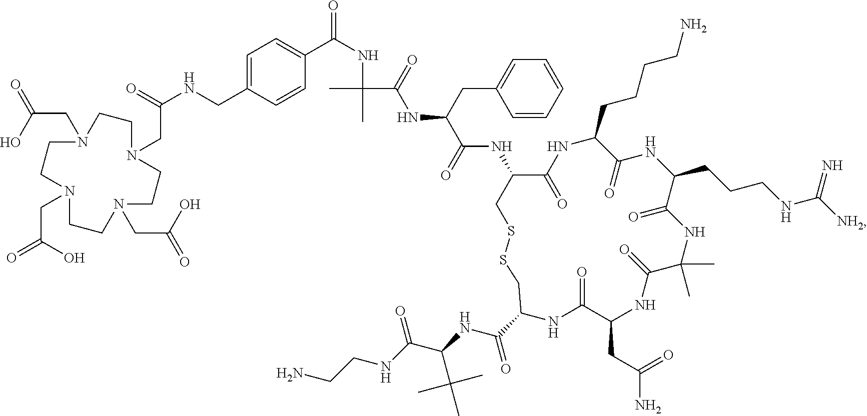 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccccc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Thr-Aib-Pcf-[Cys-Lys-Arg-Aib-Asn-Cys]-Tle-en (PSM-0222) of the following formula

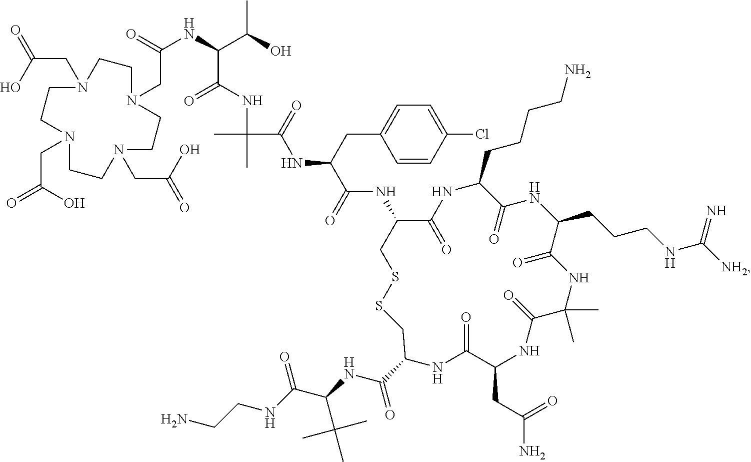 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccc(Cl)cc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Aib-Pcf-[Cys-Lys-Arg(Ac)-Aib-Asn-Cys]-Tle-Thr-NH₂ (PSM-0223) of the following formula

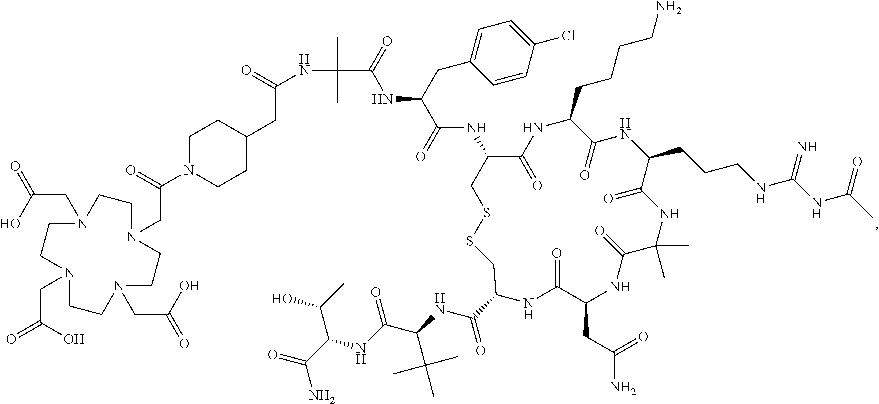 [SMILES: CC(=O)NC(=N)NCCC[C@@H]1NC(=O)[C@H](CCCCN)NC(=O)[C@@H](NC(=O)[C@H](Cc2ccc(Cl)cc2)NC(=O)C(C)(C)NC(=O)CC2CCN(C(=O)CN3CCN(CC(=O)O)CCN(CC(=O)O)CCN(CC(=O)O)CC3)CC2)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound SaPr-Aib-Phe-[Cys-Lys-Arg-Aib-Asn-Cys]-Tle-Thr-NH₂ (PSM-0224) of the following formula

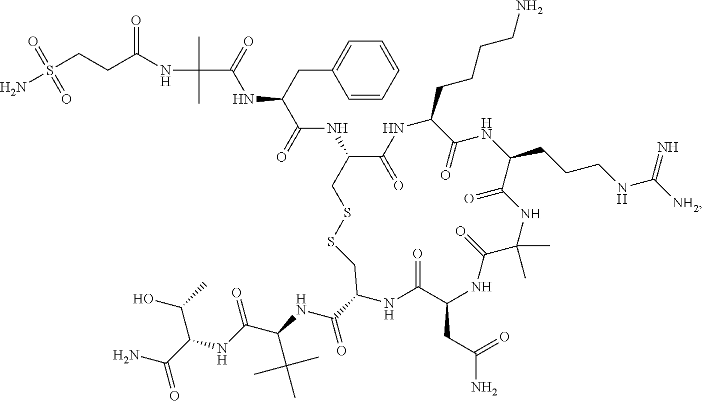 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CCS(N)(=O)=O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Har-Arg-Aib-Asn-Cys]-Tle-Thr-NH₂ (PSM-0225) of the following formula

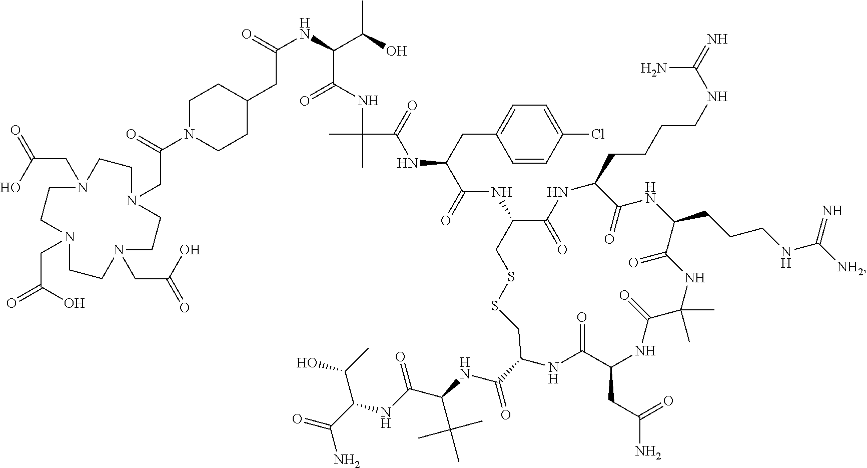 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCNC(=N)N)C(=O)N[C@@H](CCCNC(=N)N)C(=O)NC(C)(C)C(=O)N[C@@H](CC(N)=O)C(=O)N1)C(C)(C)C)C(N)=O]

compound H-Met-Thr-Ala-Phe-[Cys-Lys-Arg-Ala-Asn-Cys]-Leu-Thr-Asp-Gly-Ser-Ttds-Ttds-Ttds-Lys(Bio)-NH₂ (PSM-0226) of the following formula

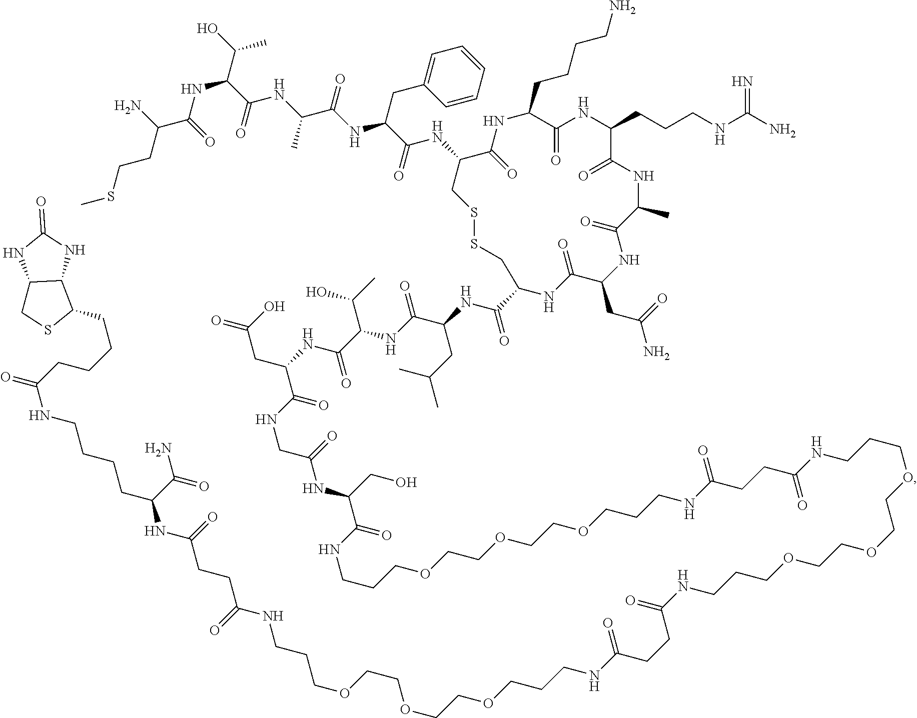 [SMILES: CSCCC(N)C(=O)N[C@H](C(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCC(=O)N[C@@H](CO)C(=O)NCCCOCCOCCOCCCNC(=O)CCC(=O)NCCCOCCOCCOCCCNC(=O)CCC(=O)NCCCOCCOCCOCCCNC(=O)CCC(=O)N[C@@H](CCCCNC(=O)CCCC[C@@H]2SC[C@@H]3NC(=O)N[C@@H]32)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-amd-Asn-Cys]-Tle-Thr-NH₂ (PSM-0227) of the following formula

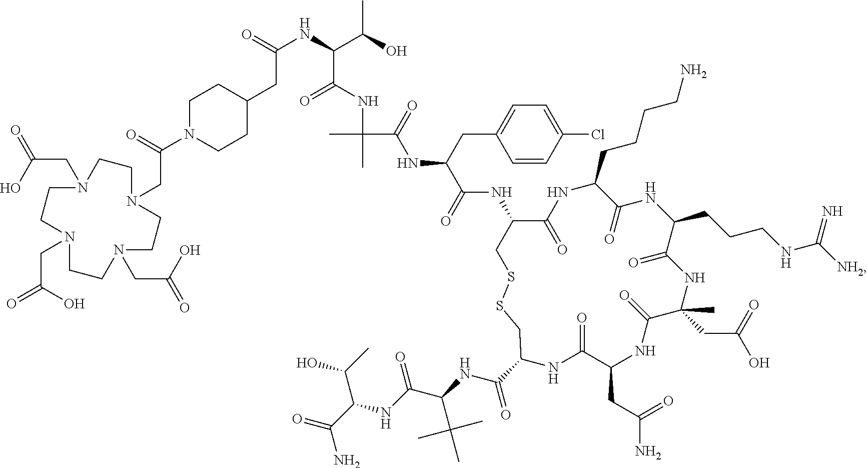 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@](C)(CC(=O)O)C(=O)N[C@@H](CC(N)=O)C(=O)N1)C(C)(C)C)C(N)=O]

compound Ac-Aib-Phe-[Cys-Lys-Arg-Aib-Asn-Cys]-Tle-Thr-NH₂ (PSM-0228) of the following formula

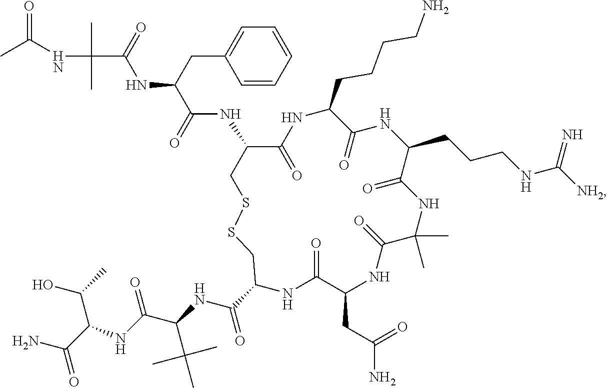 [SMILES: CC(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Pcf-[Cys-Lys-Arg-Aib-Met-Cys]-Tle-Thr-NH₂ (PSM-0229) of the following formula

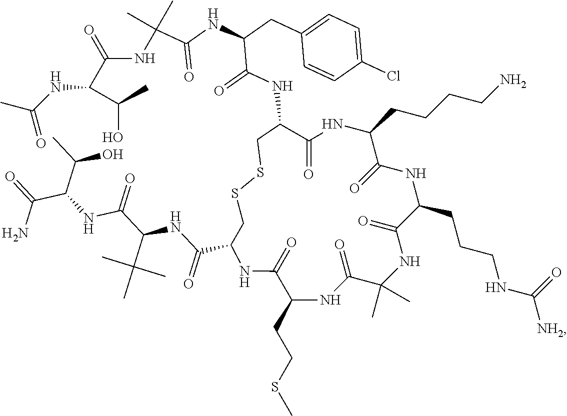 [SMILES: CSCC[C@@H]1NC(=O)C(C)(C)NC(=O)[C@H](CCCNC(N)=O)NC(=O)[C@H](CCCCN)NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC1=O]

compound Ac-Thr-Aib-Phe-[Cys-Lys-Arg-Aib-Asn-Cys]-Tle-NH₂ (PSM-0230) of the following formula

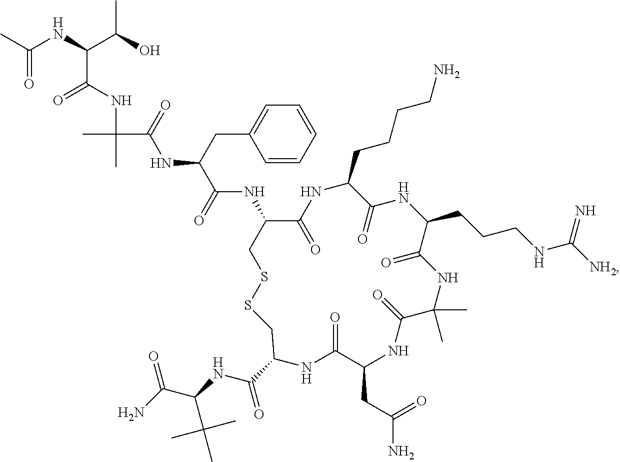 [SMILES: CC(=O)N[C@H](C(=O)NC(C)(C)C(=O)N[C@@H](Cc1ccccc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Ala-Asn-Cys]-Tle-Thr-Ttds-lys(DOTA)-NH₂ (PSM-0231) of the following formula

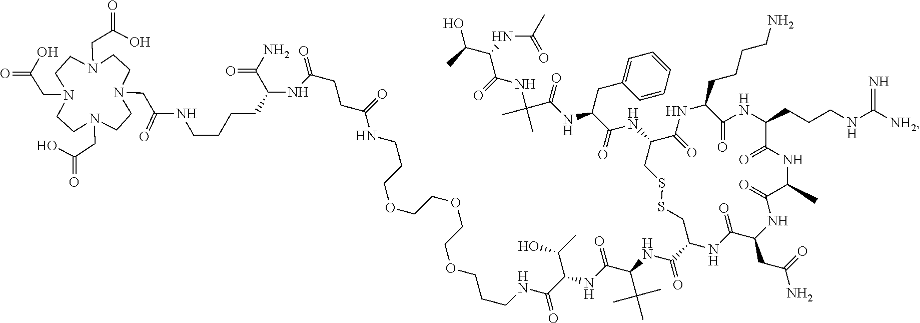 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Nmg-Asn-Cys]-Tle-Thr-NH₂ (PSM-0232) of the following formula

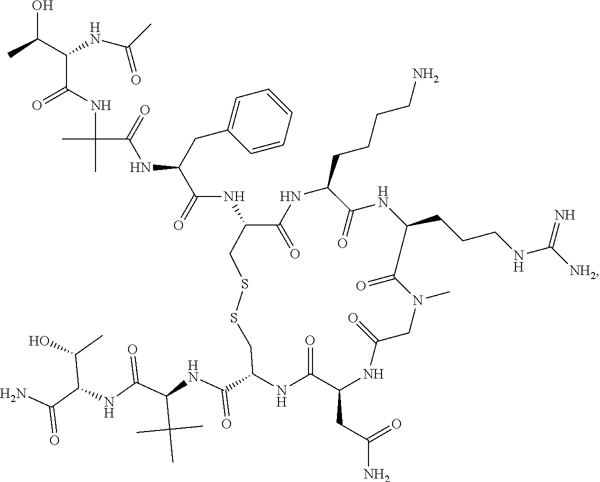 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)CN(C)C(=O)[C@H](CCCNC(=N)N)NC(=O)[C@H](CCCCN)NC1=O)[C@@H](C)O]

compound Ac-Thr-Aib-Phe-[Cys-Lys-Arg-Pam-Asn-Cys]-Tle-Thr-NH_{Z} (PSM-0233) of the following formula

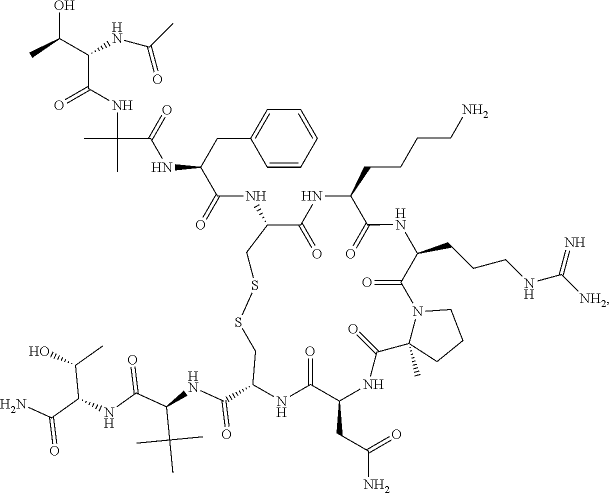 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@]2(C)CCCN2C(=O)[C@H](CCCNC(=N)N)NC(=O)[C@H](CCCCN)NC1=O)[C@@H](C)O]

compound DOTA-Ttds-Thr-Aib-Phe-[Cys-Lys-Cit-Aib-Asn-Cys]-Tle-Thr-NH₂ (PSM-0234) of the following formula

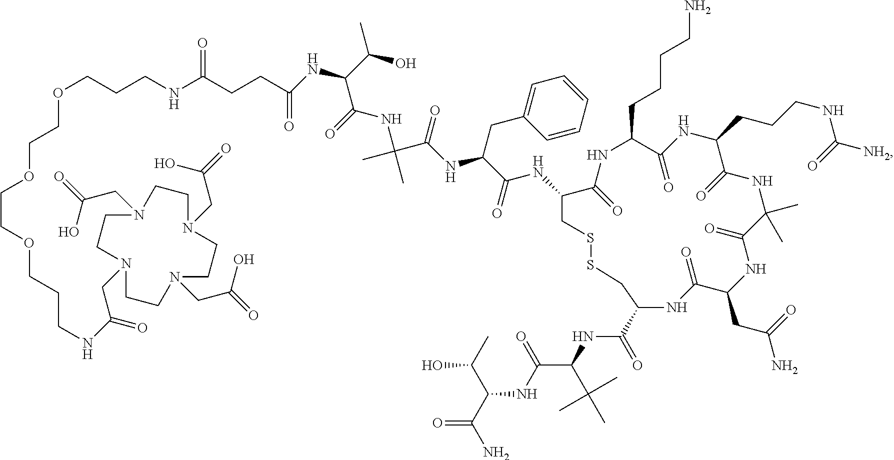 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound Ac-Thr-Aib-Phe-[Cys-Lys-Nmr-Aib-Asn-Cys]-Tle-Thr-NH₂ (PSM-0235) of the following formula

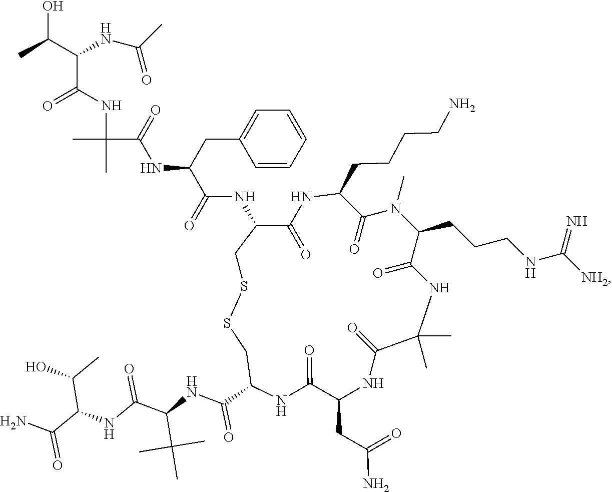 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)N(C)C(=O)[C@H](CCCCN)NC1=O)[C@@H](C)O]

compound Ac-Thr-Aib-Phf-[Cys-Nle-Gln(Gu)-Aib-Asn-Cys]-Tle-Thr-NH₂ (PSM-0236) of the following formula

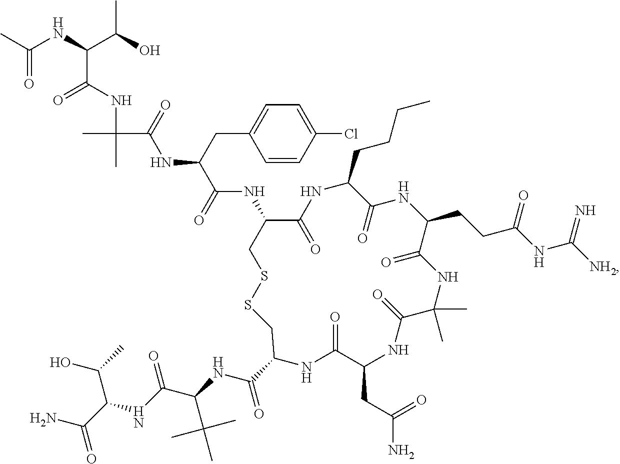 [SMILES: CCCC[C@@H]1NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCC(=O)NC(=N)N)NC1=O]

compound DOTA-Ttds-Thr-Aib-Phe-[Cys-Lys-Arg-Ala-Asn-Cys]-Tle-Thr-NH₂ (PSM-0237) of the following formula

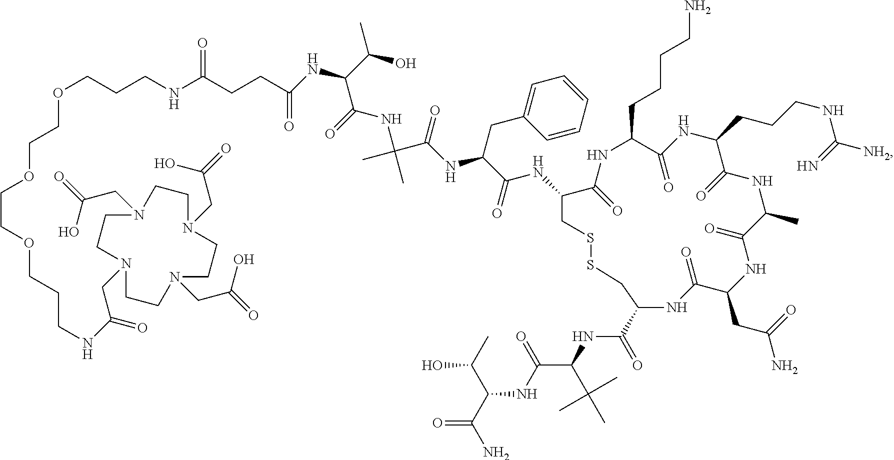 [SMILES: C[C@@H]1NC(=O)[C@H](CCCNC(=N)N)NC(=O)[C@H](CCCCN)NC(=O)[C@@H](NC(=O)[C@H](Cc2ccccc2)NC(=O)C(C)(C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC1=O]

compound Ac-Thr-Aib-Pcf-[Cys-Lys-Cit-Aib-Trp-Cys]-Tle-Thr-NH₂ (PSM-0238) of the following formula

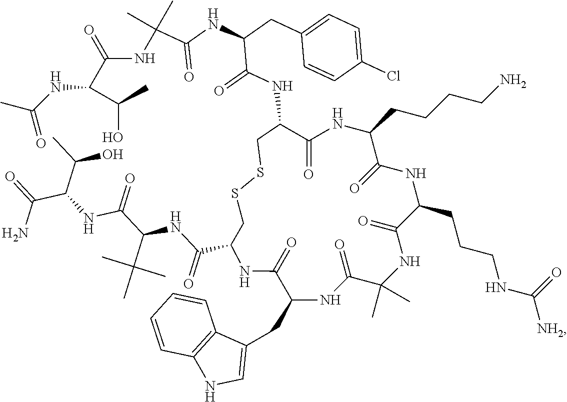 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](Cc2c[nH]c3ccccc23)NC(=O)C(C)(C)NC(=O)[C@H](CCCNC(N)=O)NC(=O)[C@H](CCCCN)NC1=O)[C@@H](C)O]

compound Hex-Thr-Aib-Pcf-[Cys-Lys-Cit-Aib-Asn-Cys]-Tle-Thr-lys(DOTA-Cmp)-NH₂ (PSM-0239) of the following formula

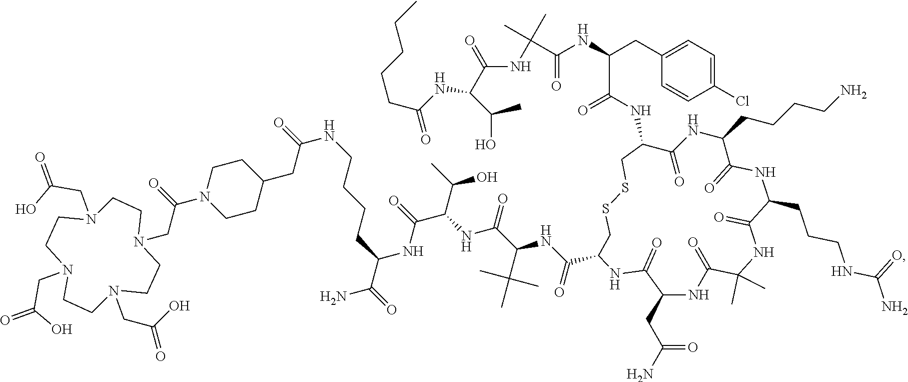 [SMILES: CCCCCC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Pcf-[Cys-Lys-Cit-Aib-Arg-Cys]-Tle-Thr-NH₂ (PSM-0240) of the following formula

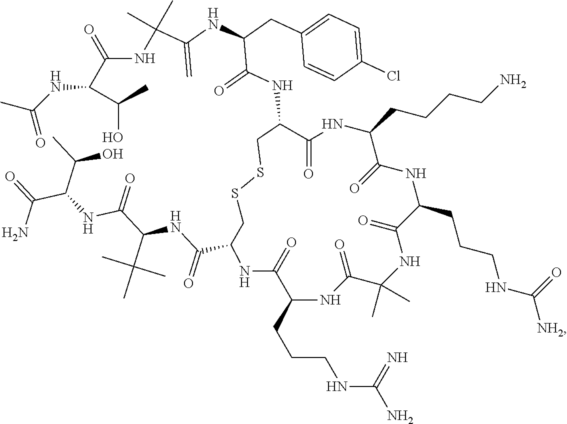 [SMILES: C=C(N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CCCNC(=N)N)NC(=O)C(C)(C)NC(=O)[C@H](CCCNC(N)=O)NC(=O)[C@H](CCCCN)NC1=O)C(C)(C)NC(=O)[C@@H](NC(C)=O)[C@@H](C)O]

compound DOTA-Cmp-Thr-Aib-Phe-[Cys-Lys-Arg-Aib-Asn-Cys]-Tle-en (PSM-0241) of the following formula

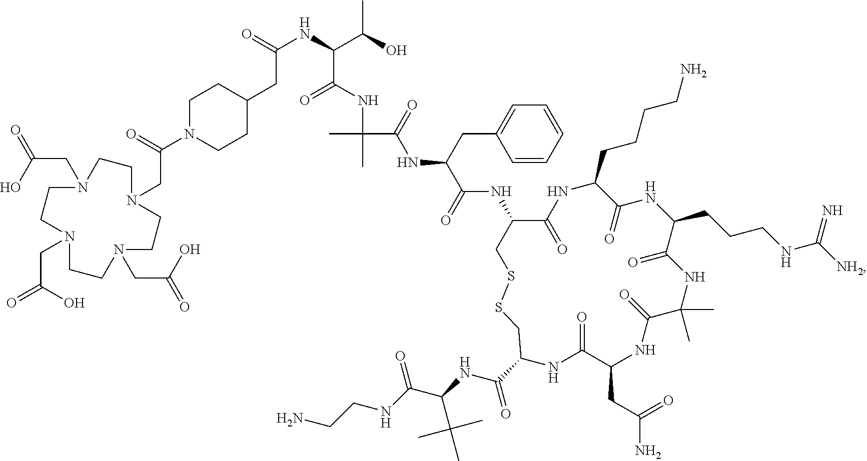 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccccc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Mcf-[Cys-Lys-Arg-Lys(DOTA-O2Oc)-Asn-Cys]-Tle-Thr-NH₂ (PSM-0243) of the following formula

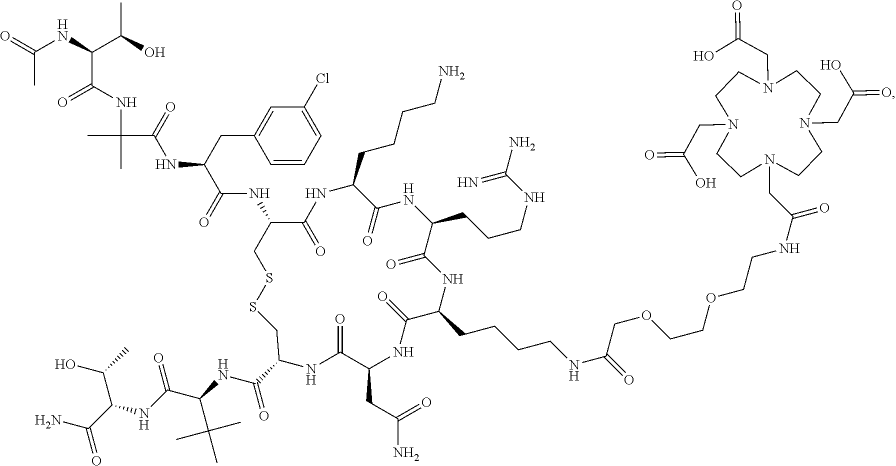 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)COCCOCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound DOTA-Cmp-Thr-Aib-Pcf-[Cys-Kip-Arg(Me)-Aib-Asn-Cys]-Tle-Thr-NH₂ (PSM-0244) of the following formula

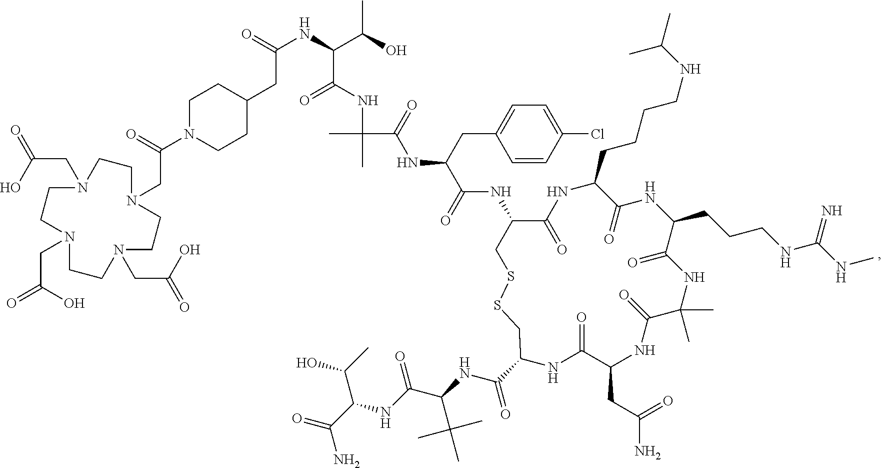 [SMILES: CNC(=N)NCCC[C@@H]1NC(=O)[C@H](CCCCNC(C)C)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound DOTA-Cmp-Thr-Aib-Pcf-[Cys-Lys-Orn-Aib-Asn-Cys]-Tle-Thr-NH₂ (PSM-0245) of the following formula

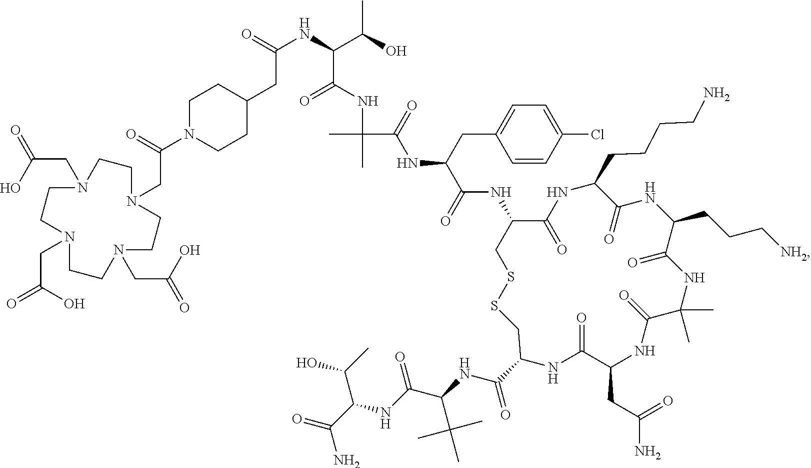 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCN)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys(Me)-Arg(Me)-ala-Asn-Cys]-Tle-Thr-NH₂ (PSM-0246) of the following formula

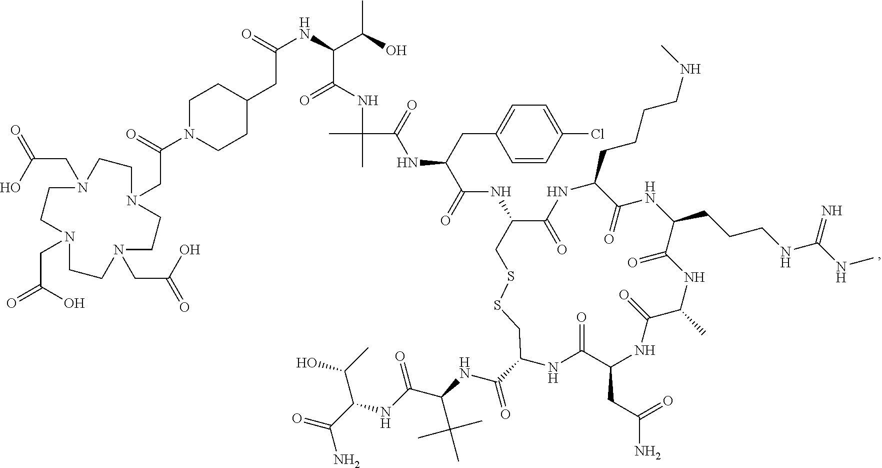 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)[C@@H](C)NC(=O)[C@H](CCCNC(=N)NC)NC1=O]

compound DOTA-Cmp-Thr-Aib-Pcf-[Cys-Lys(Me)-RMe2-Aib-Asn-Cys]-Tle-Thr-NH₂ (PSM-0247) of the following formula

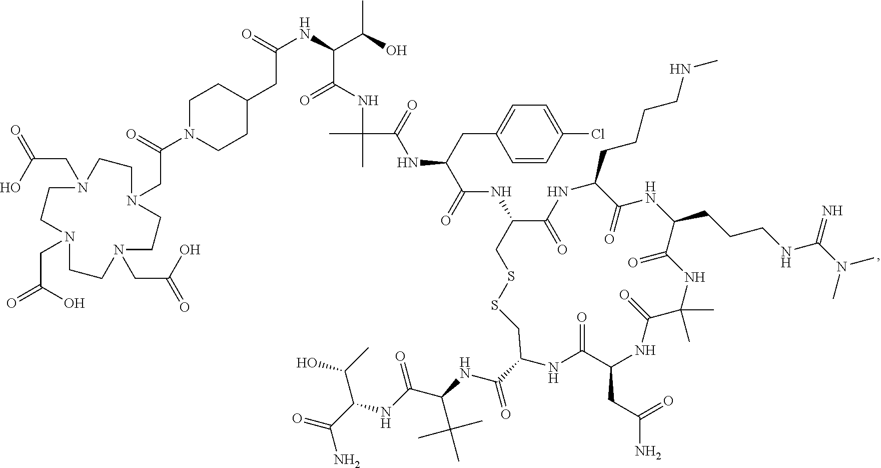 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N(C)C)NC1=O]

compound DOTA-Cmp-Aib-Pcf-[Cys-Lys-Arg-Aib-Asn-Cys]-Tle-Thr-NH₂ (PSM-0248) of the following formula

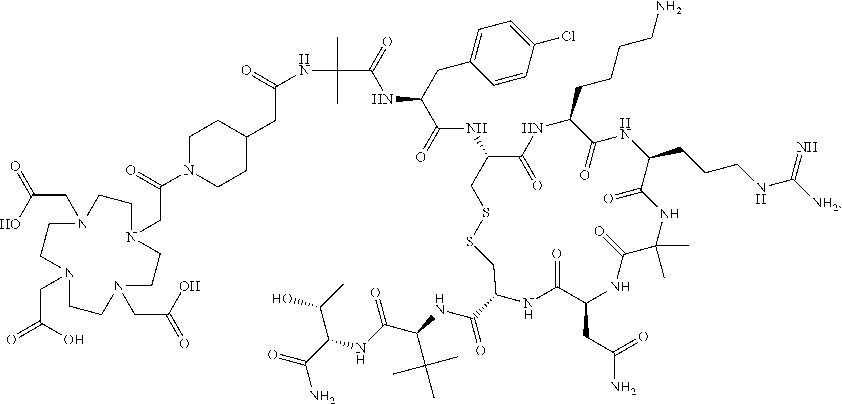 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CC2CCN(C(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-1Ni-[Cys-Lys-Arg-Aib-Asn-Cys]-Tle-Thr-Asp-NH₂ (PSM-0249) of the following formula

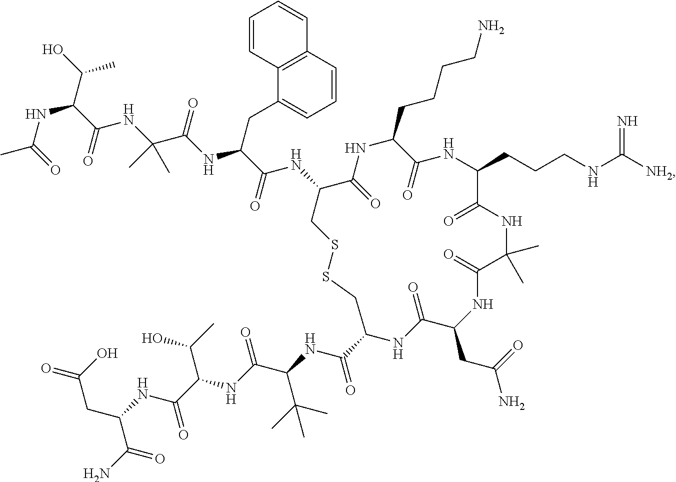 [SMILES: CC(=O)N[C@H](C(=O)NC(C)(C)C(=O)N[C@@H](Cc1cccc2ccccc12)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Aib-Asn-Cys]-Tle-Thr-Ttds-Ttds-Lys(Bio)-NH₂ (PSM-0250) of the following formula

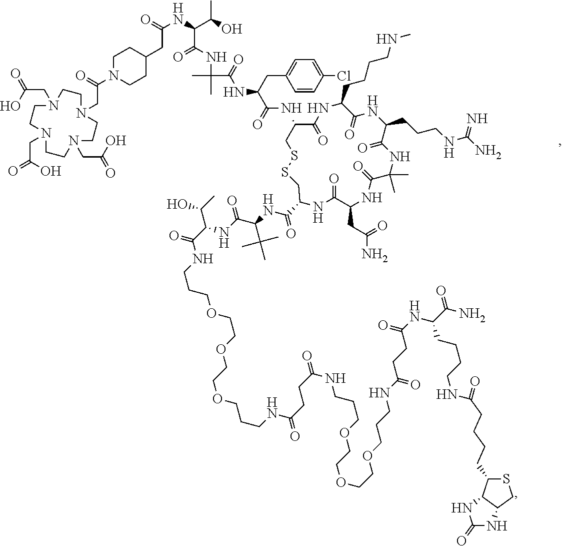 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(=O)NCCCOCCOCCOCCCNC(=O)CCC(=O)NCCCOCCOCCOCCCNC(=O)CCC(=O)N[C@@H](CCCCNC(=O)CCCC[C@@H]2SC[C@@H]3NC(=O)N[C@@H]32)C(N)=O)[C@@H](C)O)C(C)(C)C)NC(=O)[C@H](CC(N)=O)NC(=O)C(C)(C)NC(=O)[C@H](CCCNC(=N)N)NC1=O]

compound DOTA-Ttds-Thr-Ala-Phe-[Cys-Lys-Arg-Ala-Asn-Cys]-Leu-Thr-Asp-NH₂ (PSM-0251) of the following formula

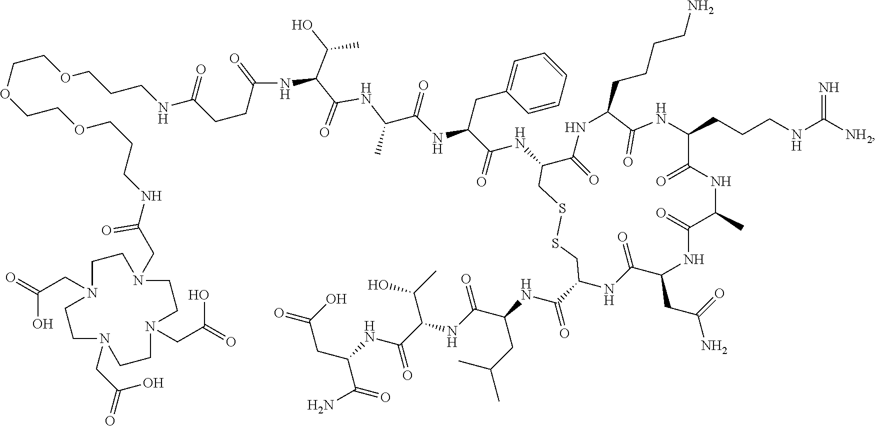 [SMILES: CC(C)C[C@H](NC(=O)[C@@H]1CSSC[C@H](NC(=O)[C@H](Cc2ccccc2)NC(=O)[C@H](C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Phe-[Cys-Lys-Cit-Aib-Asn-Cys]-Tle-Thr-Cmp-lys(DOTA)-NH₂ (PSM-0252) of the following formula

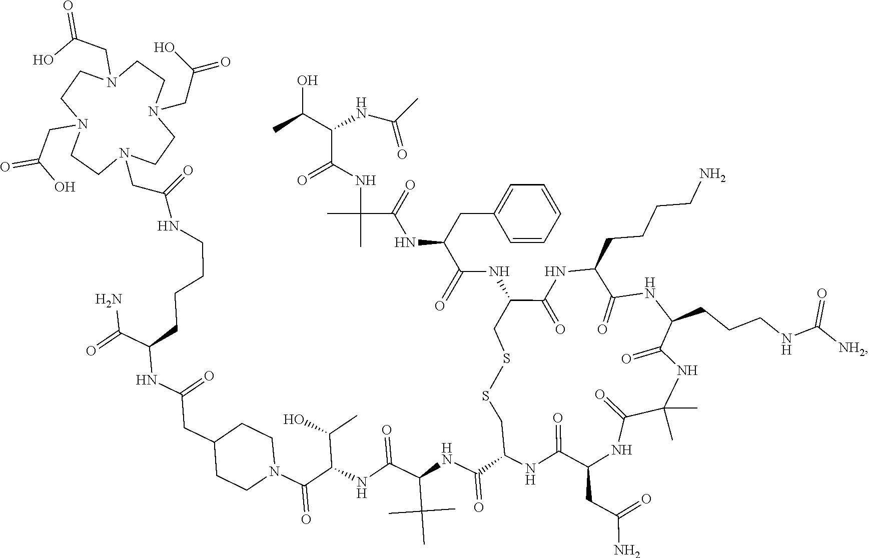 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Phe-[Cys-Lys-Glu-Aib-Asn-Cys]-Tle-Thr-Asp-NH₂ (PSM-0253) of the following formula

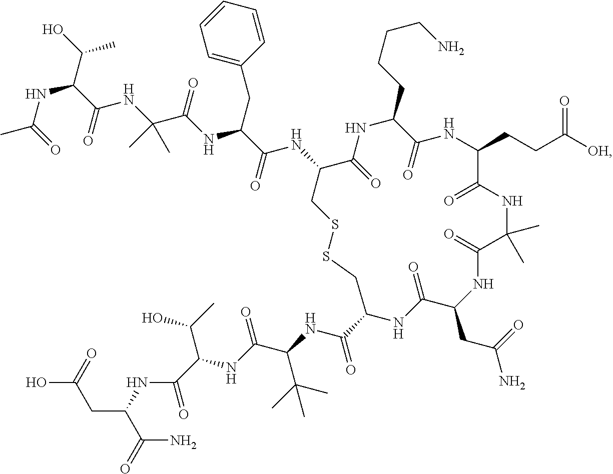 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCC(=O)O)NC(=O)[C@H](CCCCN)NC1=O)[C@@H](C)O]

compound DOTA-Cmp-Thr-Aib-Pcf-[Cys-Lys(Me)-Eew-Aib-Asn-Cys]-Tle-Thr-NH₂ (PSM-0254) of the following formula

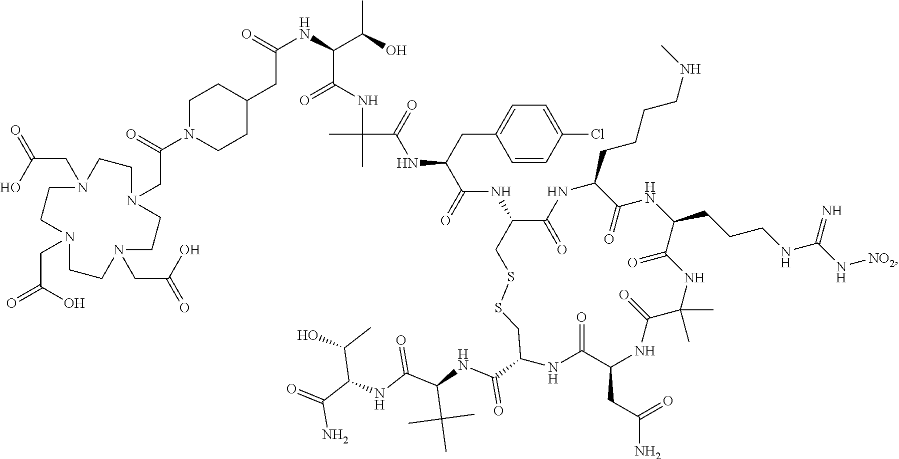 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N[N+](=O)[O-])NC1=O]

compound DOTA-Cmp-Thr-Aib-Mpa-[Cys-Lys-Cit-Aib-Asn-Cys]-Tle-Thr-NH₂ (PSM-0255) of the following formula

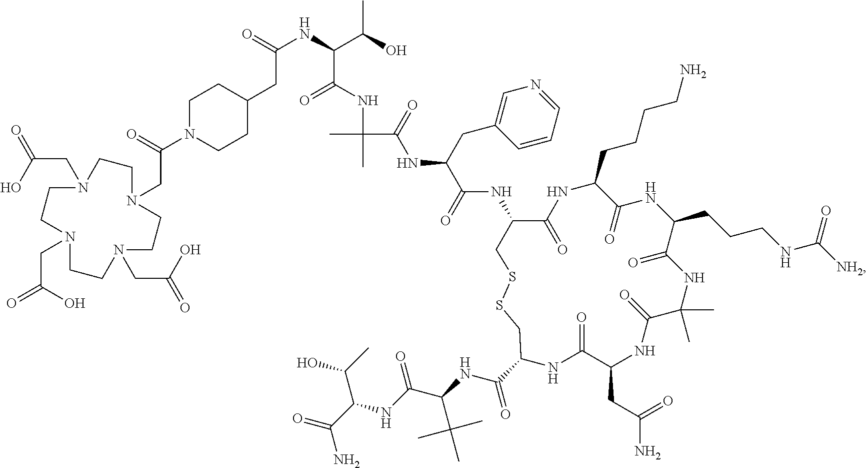 [SMILES: C[C@@H](O)[C@H](NC(=O)[C@@H](NC(=O)[C@@H]1CSSC[C@H](NC(=O)[C@H](Cc2cccnc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Cmp-Thr-ams-Pcf-[Cys-Lys-Arg-Aib-Asn-Cys]-Tle-Thr-NH₂ (PSM-0256) of the following formula

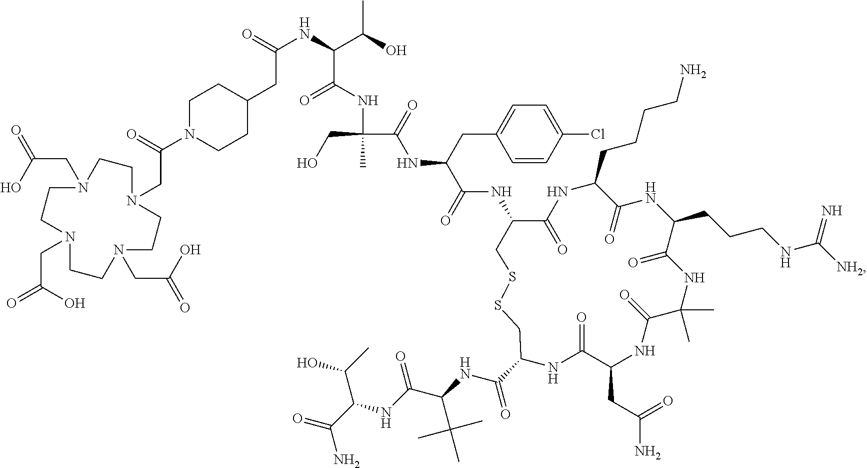 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@@](C)(CO)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Opa-[Cys-Lys(Me)-Arg(Me)-Aib-Asn-Cys]-Tle-Thr-NH₂ (PSM-0257) of the following formula

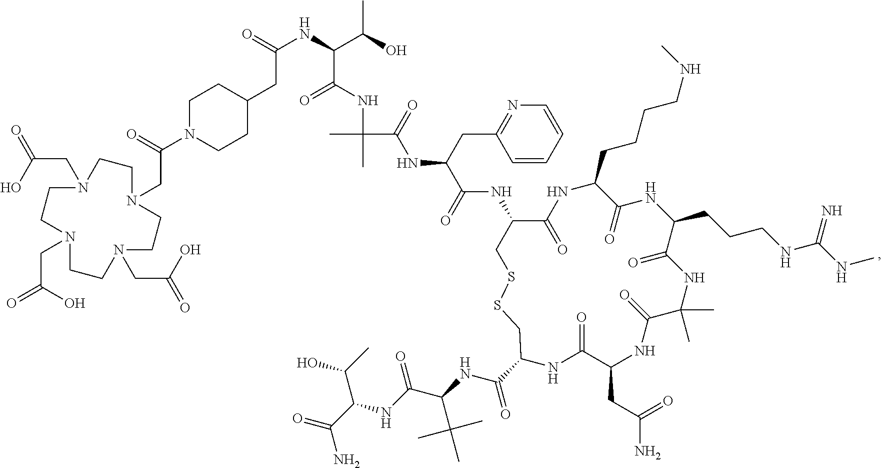 [SMILES: CNCCCC[C@@H]1NC(=O)[C@@H](NC(=O)[C@H](Cc2ccccn2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound nBuCAyl-Thr-Aib-Phe-[Cys-Lys-Opy-Aib-Asn-Cys]-Tle-Thr-Cmp-lys(DOTA)-N_{H}(PSM-0258) of the following formula

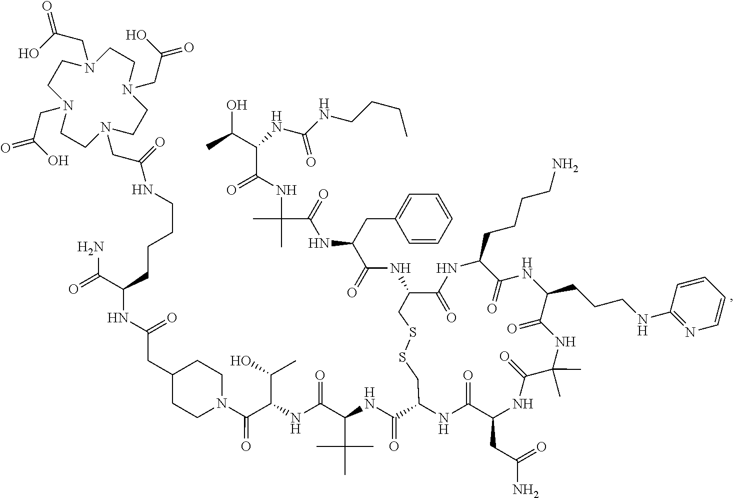 [SMILES: CCCCNC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNc2ccccn2)NC(=O)[C@H](CCCCN)NC1=O)[C@@H](C)O]

compound DOTA-Ttds-Thr-Aib-Phe-[Cys-Lys-Arg-Aib-Asn-Cys]-Tle-Thr-NH₂ (PSM-0259) of the following formula

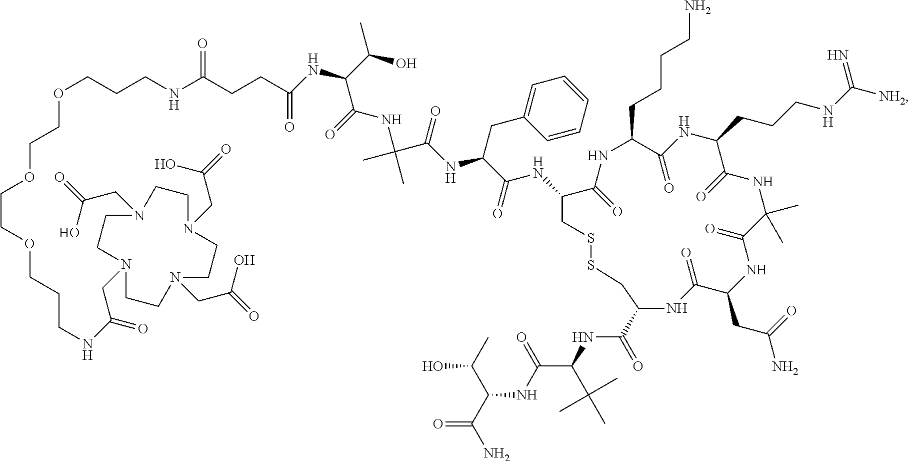 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys-Arg-glu-Asn-Cys]-Tle-Thr-NH₂ (PSM-0260) of the following formula

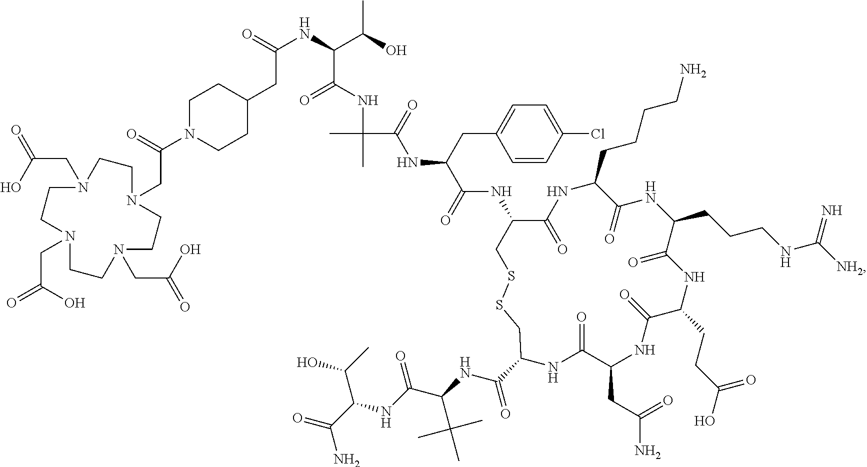 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@H](CCC(=O)O)C(=O)N[C@@H](CC(N)=O)C(=O)N1)C(C)(C)C)C(N)=O]

compound DOTA-APAc-Aib-Phe-[Cys-Lys-Arg-Aib-Asn-Cys]-Tle-Thr-NH₂ (PSM-0261) of the following formula

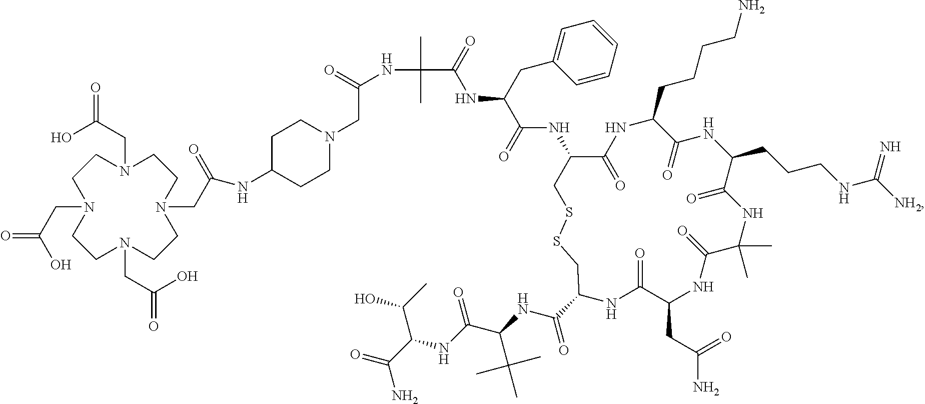 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CN2CCC(NC(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Cys-Lys-Arg-Aib-Asn-Cys]-Tle-Glu-NH₂ (PSM-0262) of the following formula

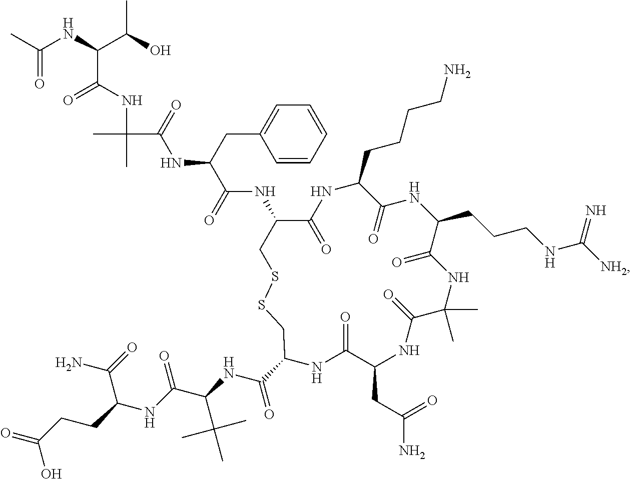 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@@H](CCC(=O)O)C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTAP-APAc-Aib-Phe-[Cys-Lys-Arg-Aib-Asn-Cys]-Tle-Thr-NH₂ (PSM-0263) of the following formula

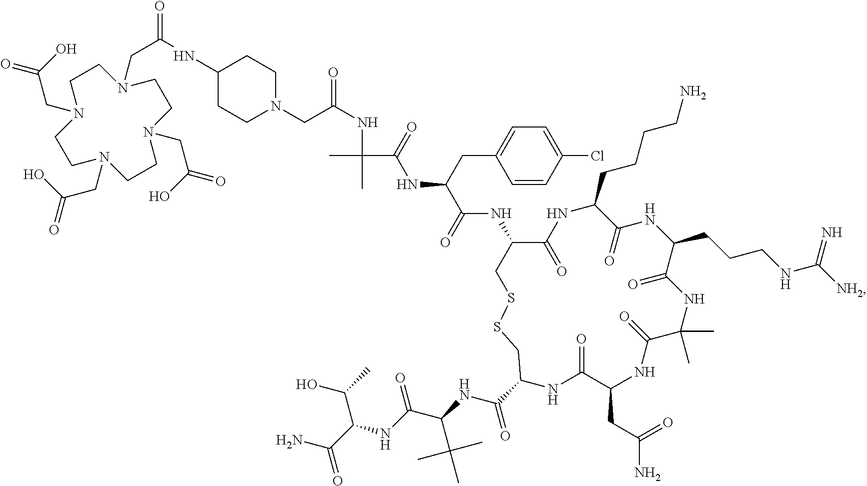 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CN2CCC(NC(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Mtf-[Cys-Lys-Arg-Aib-Asn-Cys]-Tle-Thr-NH₂ (PSM-0264) of the following formula

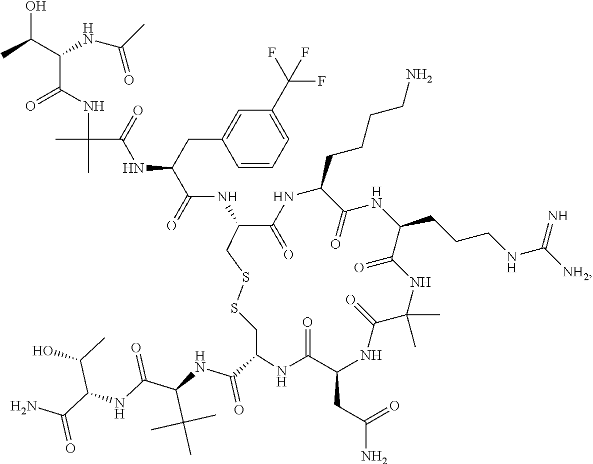 [SMILES: CC(=O)N[C@H](C(=O)NC(C)(C)C(=O)N[C@@H](Cc1cccc(C(F)(F)F)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound InDOTA-Ttds-Thr-Aib-Phe-[Cys-Lys-Arg-Ala-Asn-Cys]-Tle-Thr-NH₂ (PSM-0265) of the following formula

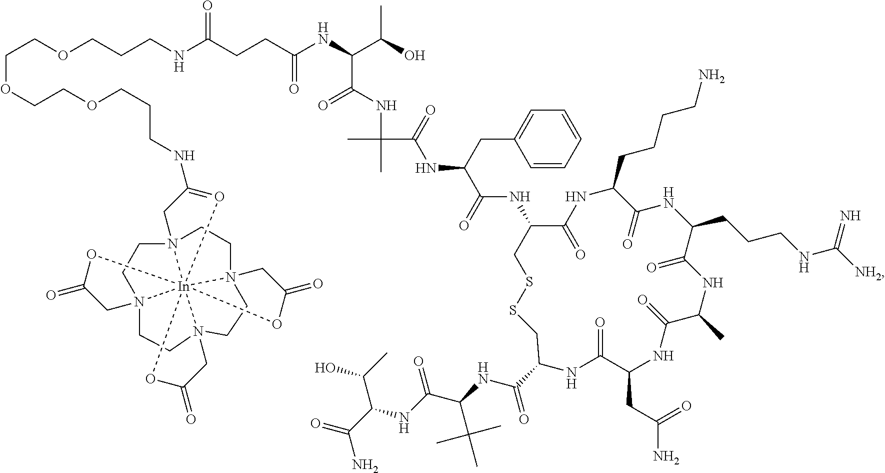

compound Ac-Thr-Ala-Phe-[Cys-Lys-Arg-Ala-Asn-Cys]-Tle-Thr-Asp-Ttds-lys(DOTA)-NH₂ (PSM-0266) of the following formula

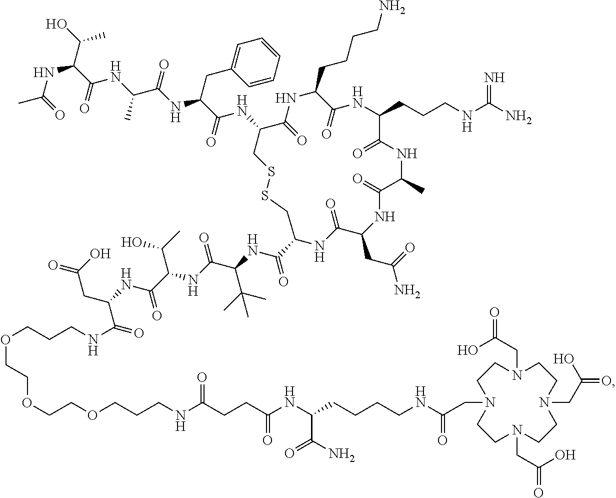 [SMILES: CC(=O)N[C@H](C(=O)N[C@@H](C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound HPA-Aib-Pcf-[Cys-Lys-Arg-Aib-Asn-Cys]-Tle-Thr-lys(DOTA)-NH₂ (PSM-0267) of the following formula

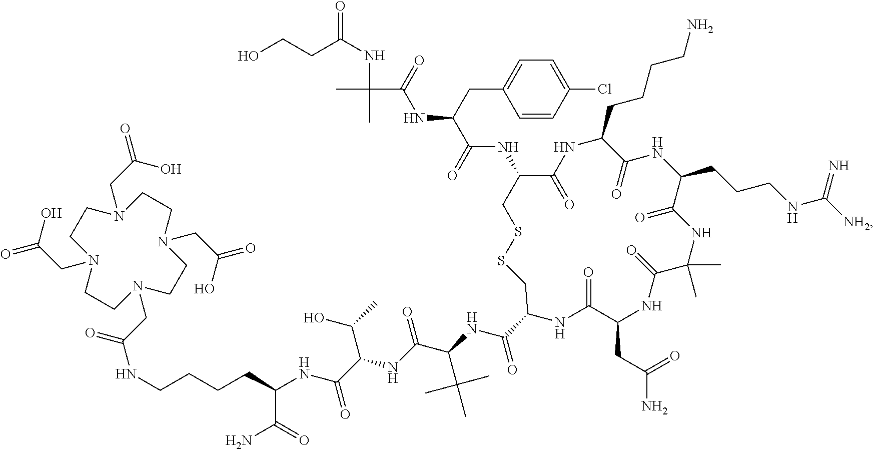 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCO)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(=O)N[C@H](CCCCNC(=O)CN1CCN(CC(=O)O)CCN(CC(=O)O)CCN(CC(=O)O)CC1)C(N)=O]

compound Hex-Thr-Aib-Phe-[Cys-Lys-Arg-Aib-Asn-Cys]-Tle-Thr-Asp-Ttds-lys (InDOTA)-NH₂ (PSM-0268) of the following formula

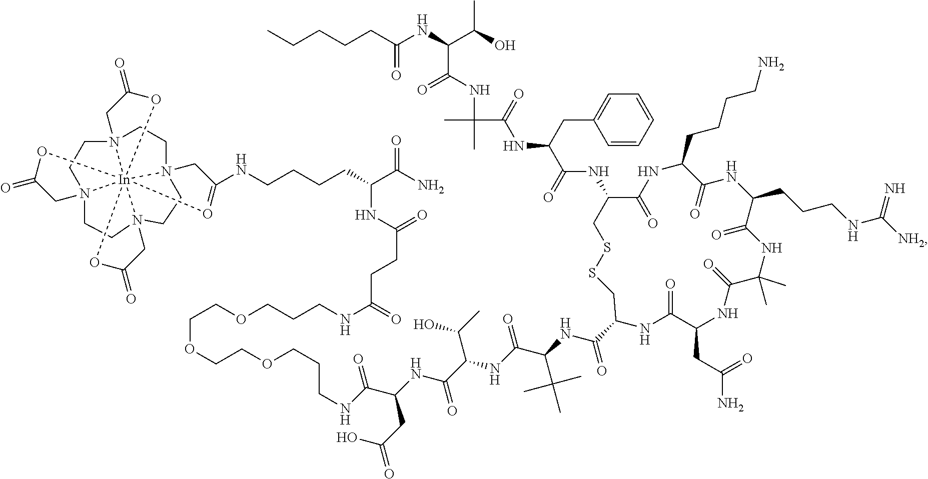

compound Ac-Thr-Aib-Pcf-[Cys-Lys-Arg-Aib-Asn-Cys]-Tle-en-DOTA (PSM-0269) of the following formula

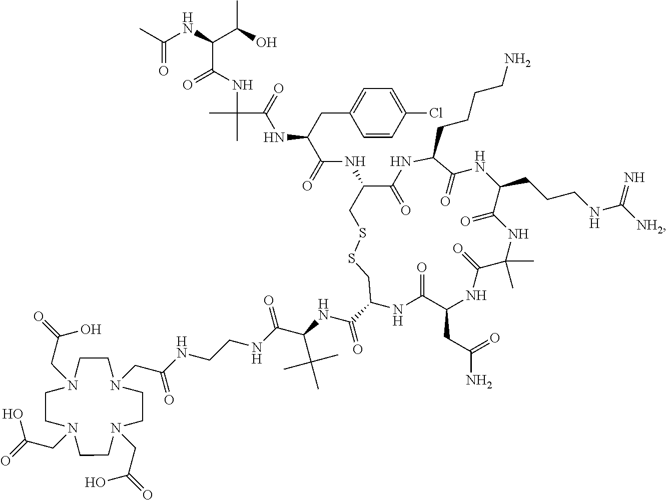 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)NCCNC(=O)CN2CCN(CC(=O)O)CCN(CC(=O)O)CCN(CC(=O)O)CC2)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Pamb-Aib-Phe-[Cys-Lys-Arg-Aib-Asn-Cys]-Tle-Thr-NH₂ (PSM-0270) of the following formula

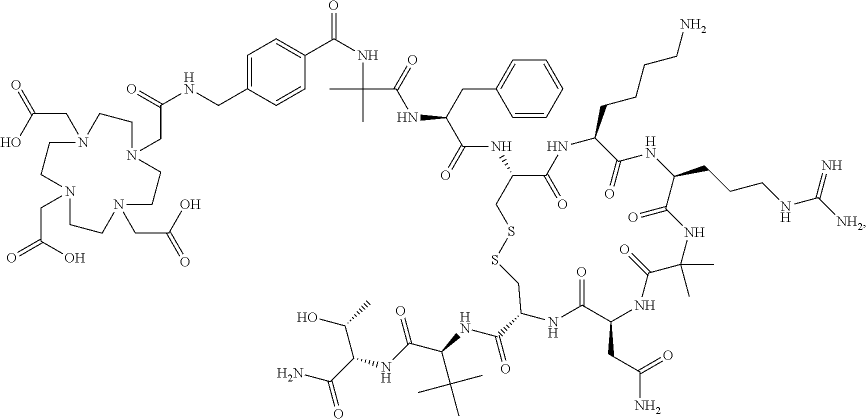 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)c2ccc(CNC(=O)CN3CCN(CC(=O)O)CCN(CC(=O)O)CCN(CC(=O)O)CC3)cc2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound AF488Ahx-Ttds-Ttds-Thr-Aib-Phe-[Cys-Lys-Arg-Aib-Asn-Cys]-Tle-Thr-Asp-NH₂ (PSM-0272) of the following formula

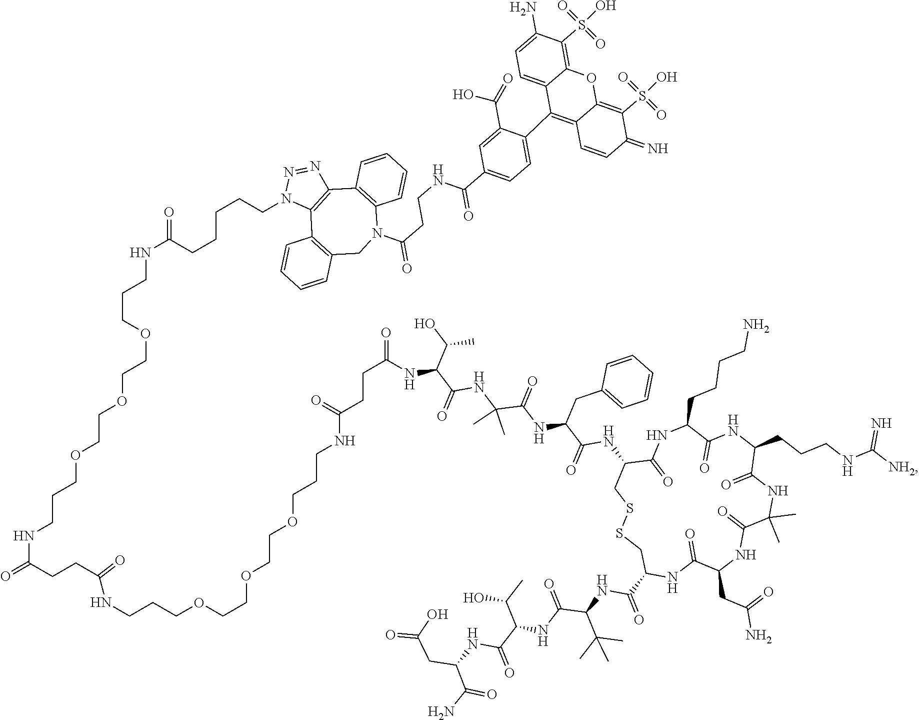 [SMILES: C[C@@H](O)[C@H](NC(=O)CCC(=O)NCCCOCCOCCOCCCNC(=O)CCC(=O)NCCCOCCOCCOCCCNC(=O)CCCCCn1nnc2c1-c1ccccc1CN(C(=O)CCNC(=O)c1ccc(-c3c4ccc(=N)c(S(=O)(=O)O)c-4oc4c(S(=O)(=O)O)c(N)ccc34)c(C(=O)O)c1)c1ccccc1-2)C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Pamb-Aib-Pcf-[Cys-Lys-Arg-Aib-Asn-Cys]-Tle-en (PSM-0273) of the following formula

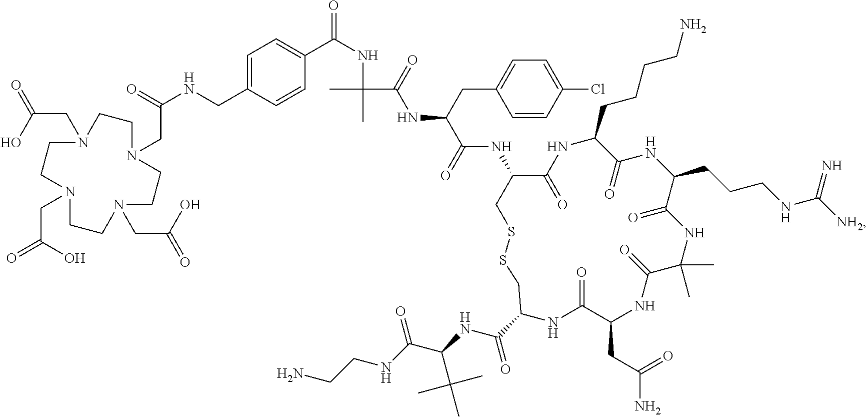 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccc(Cl)cc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Eaa-[Cys-Lys-Cit-Aib-Asn-Cys]-Tle-Thr-NH₂ (PSM-0274) of the following formula

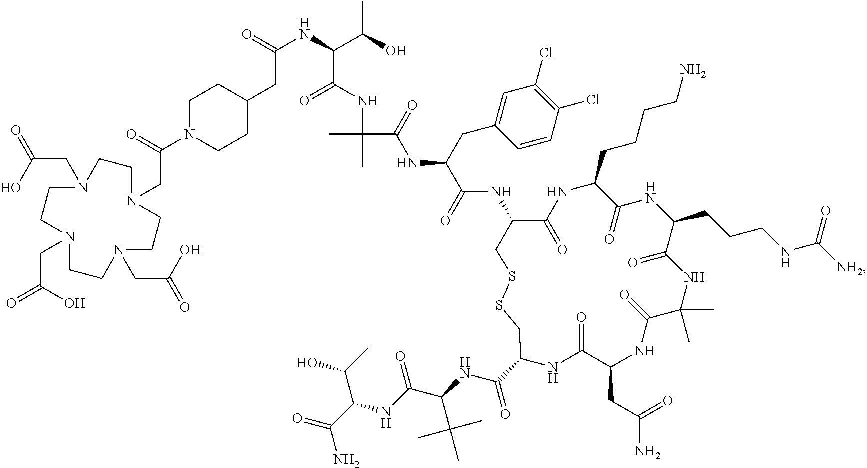 [SMILES: C[C@@H](O)[C@H](NC(=O)[C@@H](NC(=O)[C@@H]1CSSC[C@H](NC(=O)[C@H](Cc2ccc(Cl)c(Cl)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound Ac-Thr-Aib-Mcf-[Cys-Lys-Cit-Aib-Asn-Cys]-Tle-Thr-Cmp-lys(DOTA)-NH₂ (PSM-0275) of the following formula

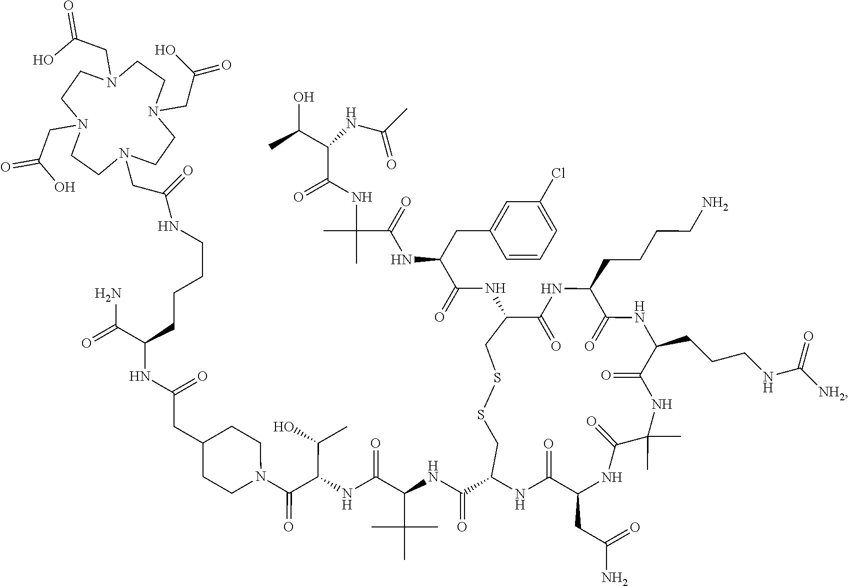 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound DOTA-Cmp-Thr-Glu-Pcf-[Cys-Lys-Arg-Aib-Asn-Cys]-Tle-Thr-NH₂ (PSM-0276) of the following formula

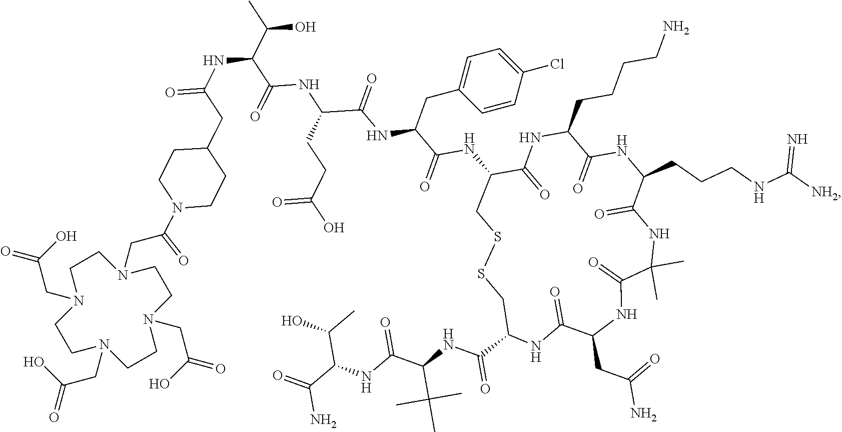 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@H](CCC(=O)O)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound InDOTA-Ttds-Thr-Aib-Phe-[Cys-Lys-Arg-Ala-Asn-Cys]-Tle-Thr-Asp-NH₂ (PSM-0277) of the following formula

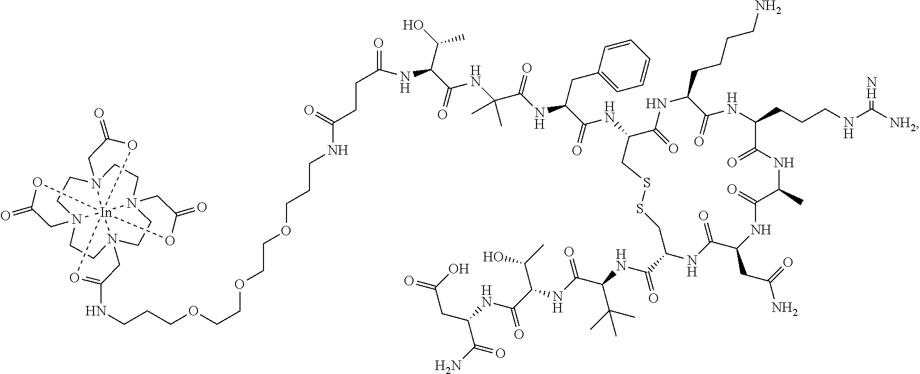

compound Ac-Thr-Aib-Pnf-[Cys-Lys-Arg-Aib-Asn-Cys]-Tle-Thr-NH₂ (PSM-0278) of the following formula

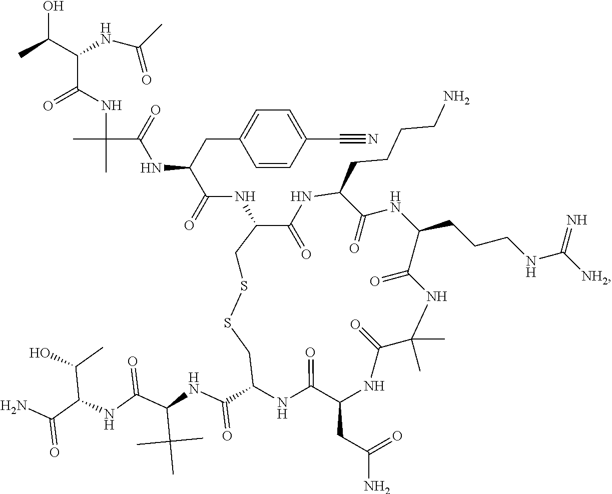 [SMILES: CC(=O)N[C@H](C(=O)NC(C)(C)C(=O)N[C@@H](Cc1ccc(C#N)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Ser-Cys]-Tle-Thr-NH₂ (PSM-0279) of the following formula

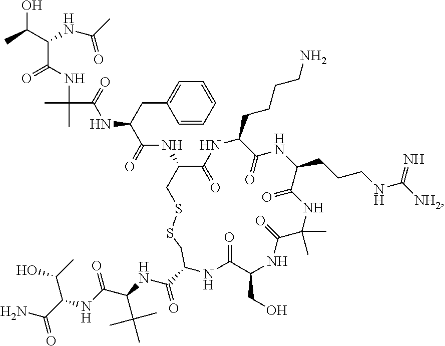 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CO)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Cit-Aib-Asn-Cys]-Tle-Thr-Asp-NH₂ (PSM-0280) of

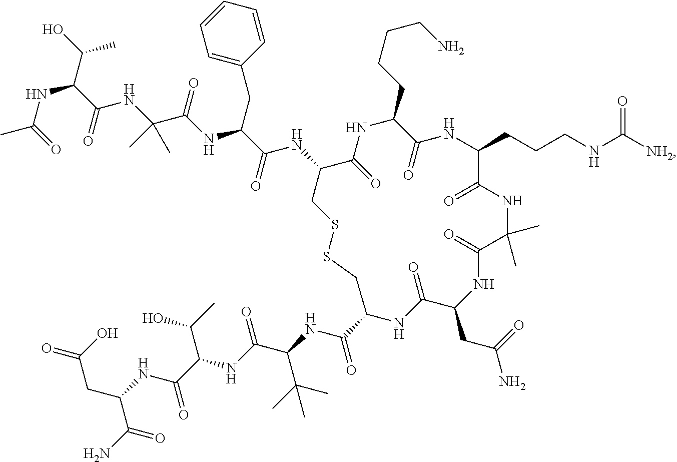 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Pcf-[Cys-Lys-Arg-Aib-Asn-Cys]-Tle-NH₂ (PSM-0281) of the following formula

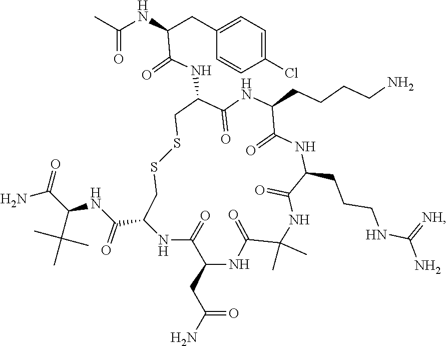 [SMILES: CC(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Aib-Pcf-[Cys-Lys-Arg-Lys(DOTA-APAc)-Asn-Cys]-Tle-Thr-NH₂ (PSM-0282) of the following formula

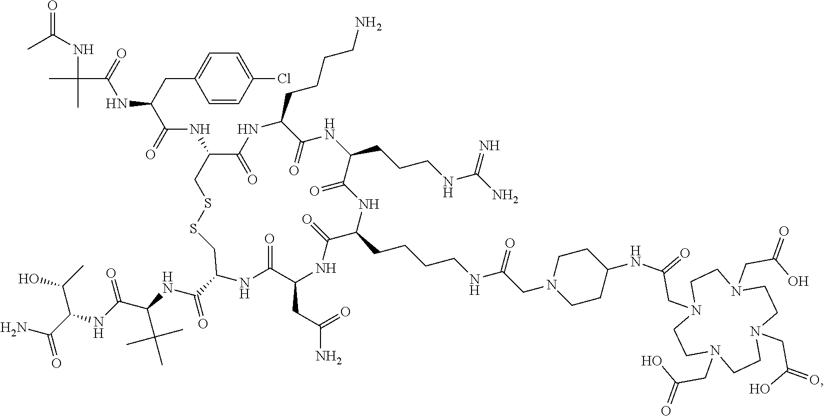 [SMILES: CC(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CN2CCC(NC(=O)CN3CCN(CC(=O)O)CCN(CC(=O)O)CCN(CC(=O)O)CC3)CC2)NC(=O)[C@H](CCCNC(=N)N)NC(=O)[C@H](CCCCN)NC1=O]

compound DOTA-Pamb-Aib-Pcf-[Cys-Lys-Arg-Aib-Asn-Cys]-Tle-Thr-NH₂ (PSM-0283) of the following formula

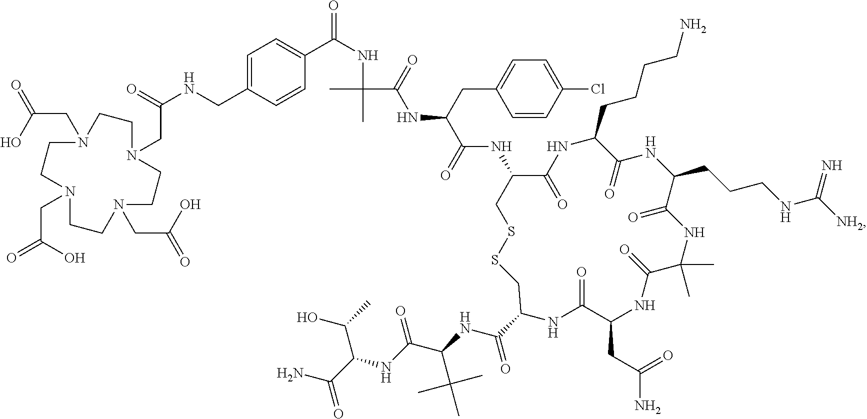 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)c2ccc(CNC(=O)CN3CCN(CC(=O)O)CCN(CC(=O)O)CCN(CC(=O)O)CC3)cc2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Mcf-[Cys-Lys-Cit-Lys(DOTA-020c)-Asn-Cys]-Tle-Thr-NH₂ (PSM-0284) of the following formula

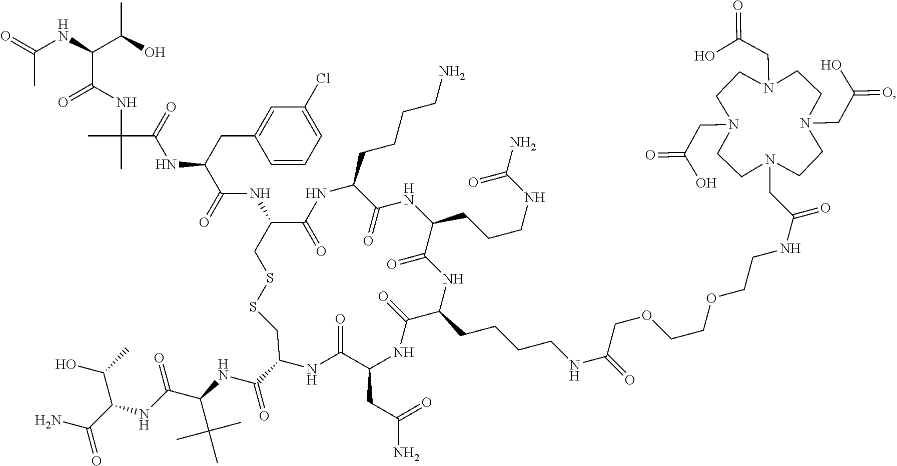 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)COCCOCCNC(=O)CN2CCN(CC(=O)O)CCN(CC(=O)O)CCN(CC(=O)O)CC2)NC(=O)[C@H](CCCNC(N)=O)NC(=O)[C@H](CCCCN)NC1=O)[C@@H](C)O]

compound Ac-Thr-Aib-Phe-[Cys-Lys-Arg-Aib-Asn-Cys]-Tle-Thr-Ttds-lys(DOTA)-NH₂ (PSM-0285) of the following formula

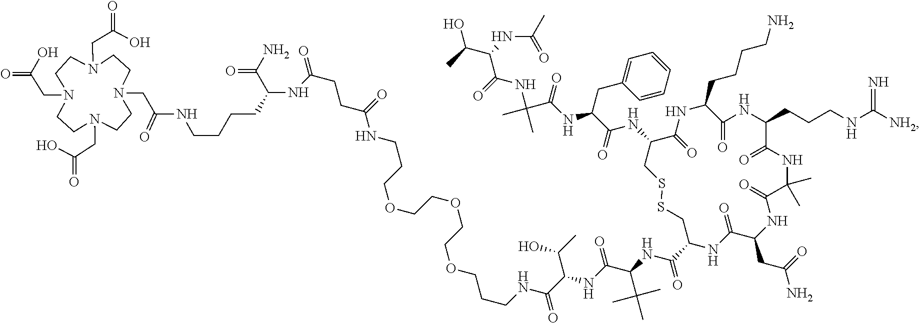 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Ala-Phe-[Cys-Lys-Arg-Ala-Pro-Cys]-Nle-Thr-Asp-NH₂ (PSM-0286) of the following formula

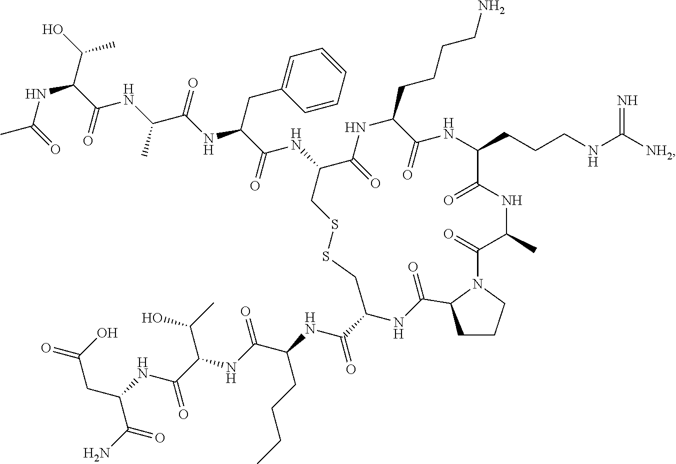 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N2CCC[C@H]2C(=O)N1)C(=O)N[C@H](C(=O)N[C@@H](CC(=O)O)C(N)=O)[C@@H](C)O]

compound Ac-Thr-Ala-Phe-[Cys-Lys-Arg-Ala-Asn-Cys]-Nle-Thr-Asp-NH₂ (PSM-0287) of the following formula

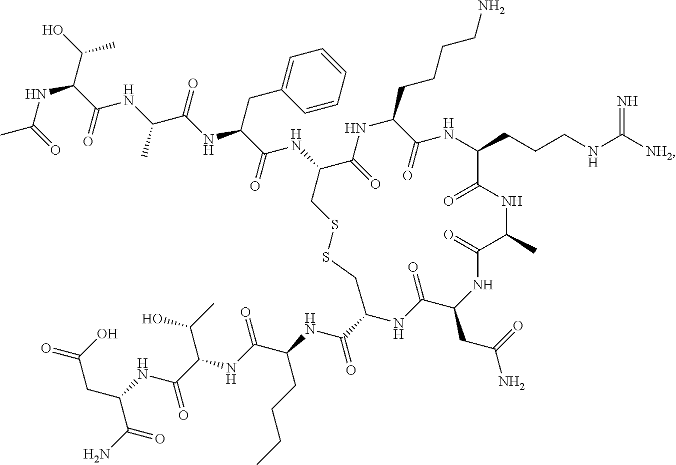 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Phe-[Cys-Lys-Arg-Aib-Asn-Cys]-Tle-Thr-lys(DOTA)-NH₂ (PSM-0288) of the following formula

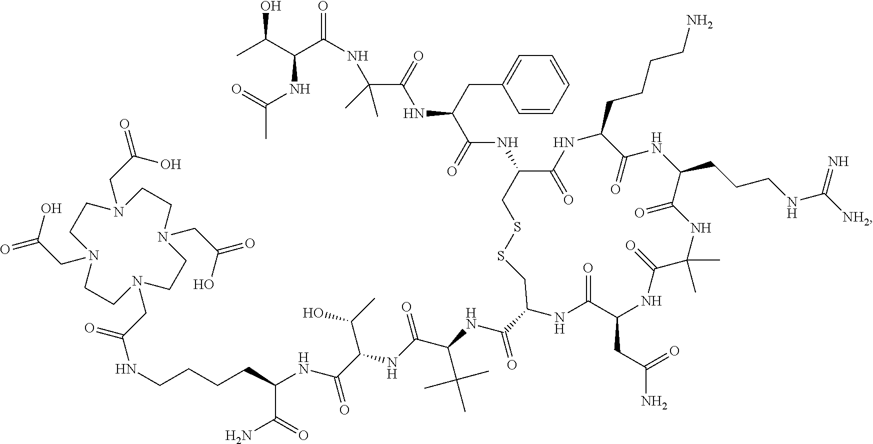 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Hex-Thr-Aib-Pho-[Cys-Lys-Gln-Aib-Asn-Cys]-Tle-Thr-Ttds-lys(DOTA)-NH₂ (PSM-0289) of the following formula

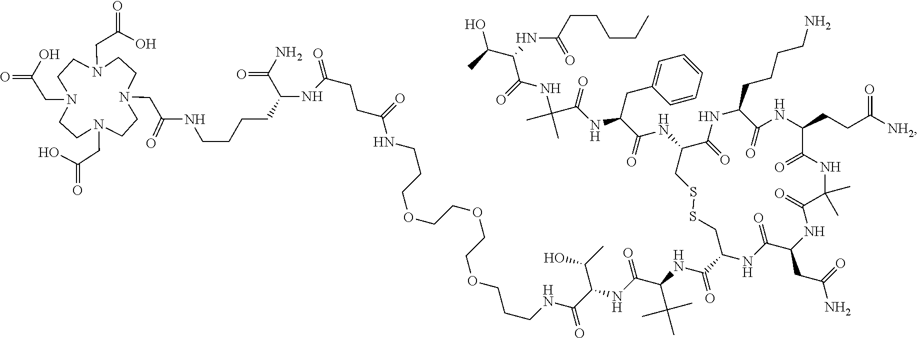 [SMILES: CCCCCC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCC(N)=O)NC(=O)[C@H](CCCCN)NC1=O)[C@@H](C)O]

compound DOTA-Aib-Pcf-[Cys-Lys-Arg-Aib-Asn-Cys]-Tle-Thr-NH₂ (PSM-0290) of the following formula

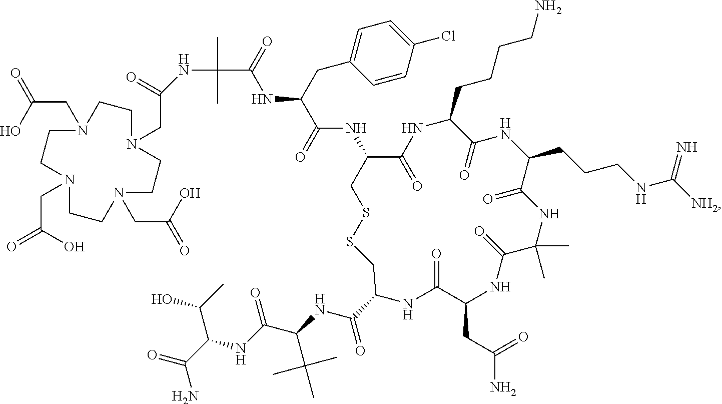 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Cys-Glu-Arg-Ala-Asn-Cys]-Tle-Thr-Asp-NH₂ (PSM-0291) of the following formula

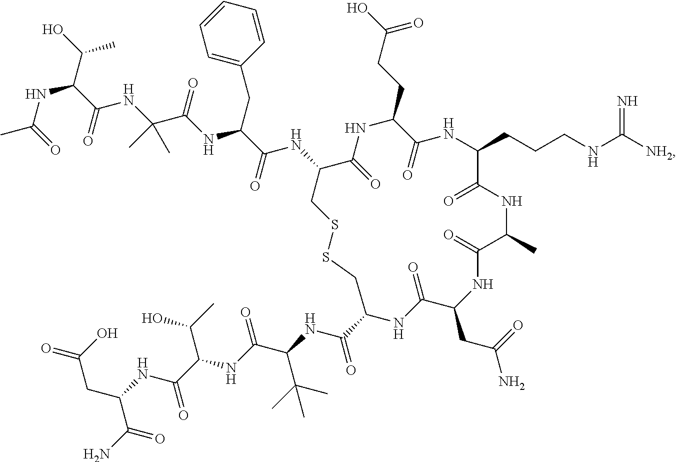 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCC(=O)O)NC1=O)[C@@H](C)O]

compound Iva-Aib-Phe-[Cys-Lys-Arg-Aib-Asn-Cys]-Tle-Thr-lys(DOTA)-NH₂ (PSM-0292) of the following formula

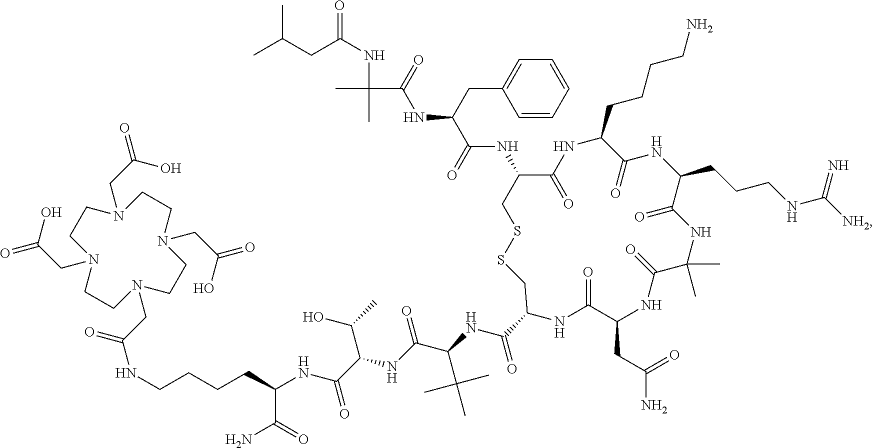 [SMILES: CC(C)CC(=O)NC(C)(C)C(=O)N[C@@H](Cc1ccccc1)C(=O)N[C@H]1CSSC[C@@H](C(=O)N[C@H](C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Pcf-[Cys-Lys-Cit-Aib-Ser-Cys]-Tle-Thr-NH₂ (PSM-0293) of the following formula

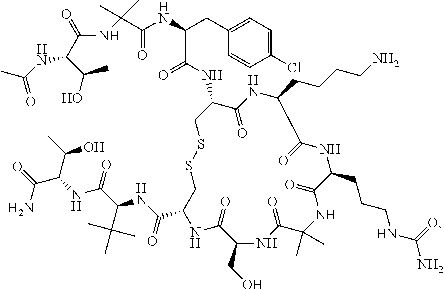 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CO)NC(=O)C(C)(C)NC(=O)[C@H](CCCNC(N)=O)NC(=O)[C@H](CCCCN)NC1=O)[C@@H](C)O]

compound Ac-Thr-Aib-Pff-[Cys-Lys-Arg-Aib-Asn-Cys]-Tle-Thr-NH₂ (PSM-0294) of the following formula

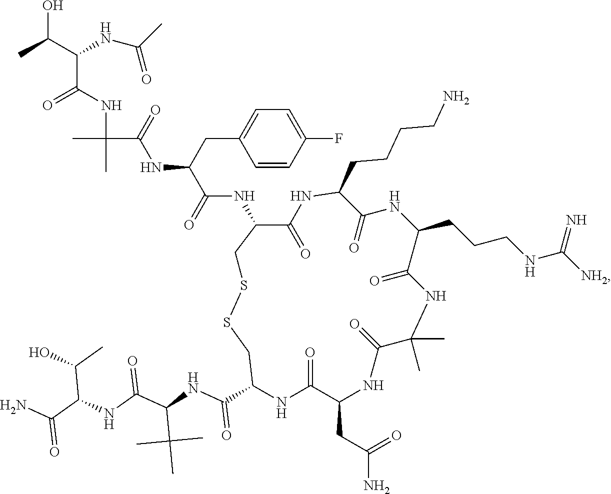 [SMILES: CC(=O)N[C@H](C(=O)NC(C)(C)C(=O)N[C@@H](Cc1ccc(F)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Ala-Asn-Cys]-Tle-Thr-Asp-Ttds-lys(DOTA)-NH₂ (PSM-0295) of the following formula

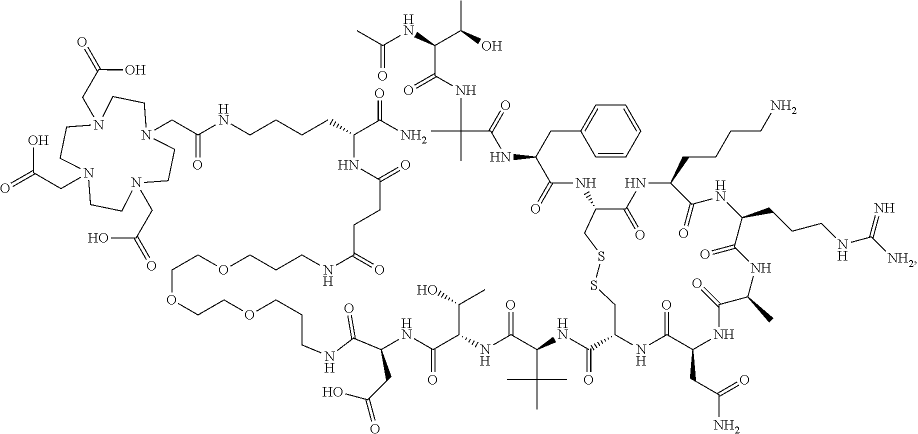 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Mpa-[Cys-Lys(Me)-Arg(Me)-Aib-Asn-Cys]-Tle-Thr-NH₂ (PSM-0296) of the following formula

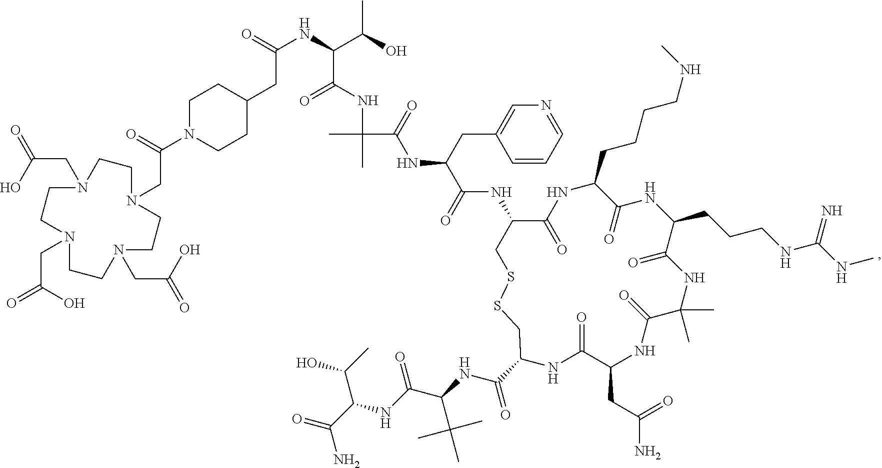 [SMILES: CNCCCC[C@@H]1NC(=O)[C@@H](NC(=O)[C@H](Cc2cccnc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound Ac-Thr-Aib-Pho-[Cys-Lys-Arg-Aib-Asn-Cys]-Tle-Gab-NH₂ (PSM-0297) of the following formula

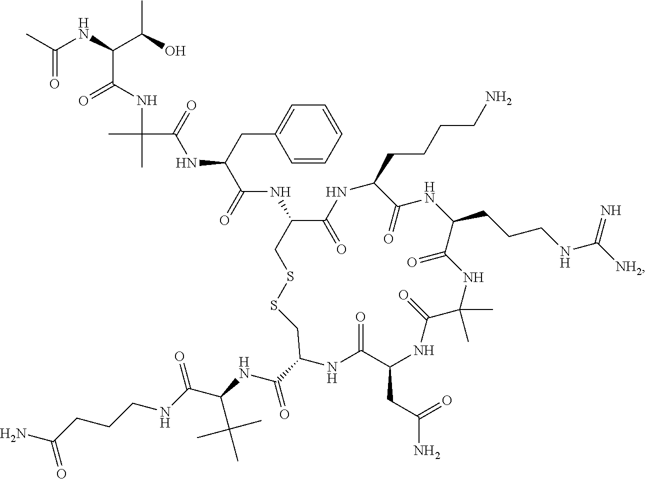 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)NCCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Pcf-[Cys-Lys-Arg(Ac)-Aib-Asn-Cys]-Tle-Thr-Cmp-lys(DOTA)-NH₂ (PSM-0298) of the following formula

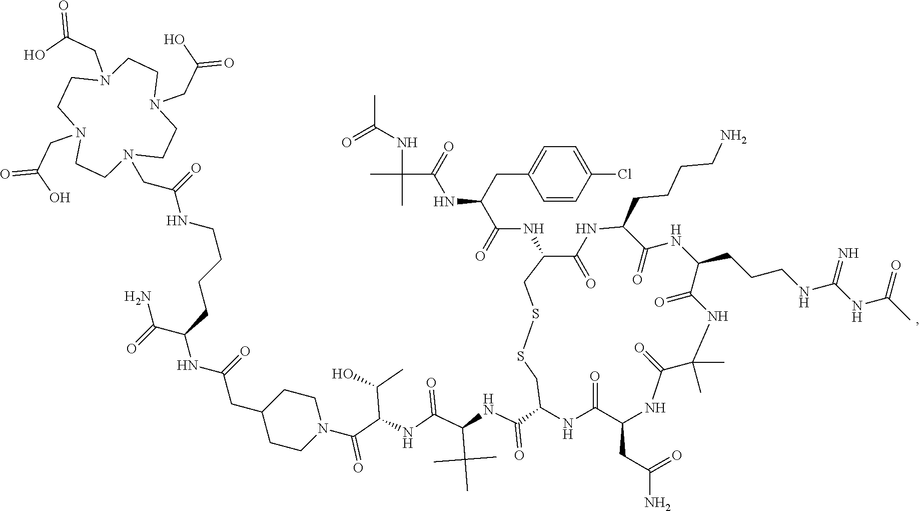 [SMILES: CC(=O)NC(=N)NCCC[C@@H]1NC(=O)[C@H](CCCCN)NC(=O)[C@@H](NC(=O)[C@H](Cc2ccc(Cl)cc2)NC(=O)C(C)(C)NC(C)=O)CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC1=O]

compound Ac-Ala-Phe-[Cys-Lys-Arg-Ala-Asn-Cys]-Leu-Thr-Asp-Ttds-lys(DOTA)-NH₂ (PSM-0299) of the following formula

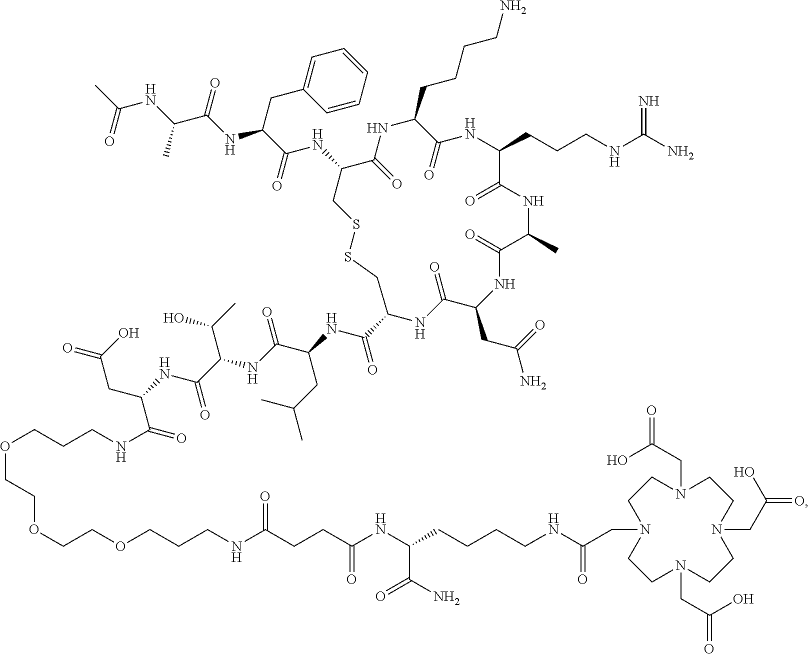 [SMILES: CC(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O]

compound Ac-Thr-Aib-Phe-[Cys-Lys-Arg-Aib-Asn-Cys]-Tle-OH (PSM-0300) of the following formula

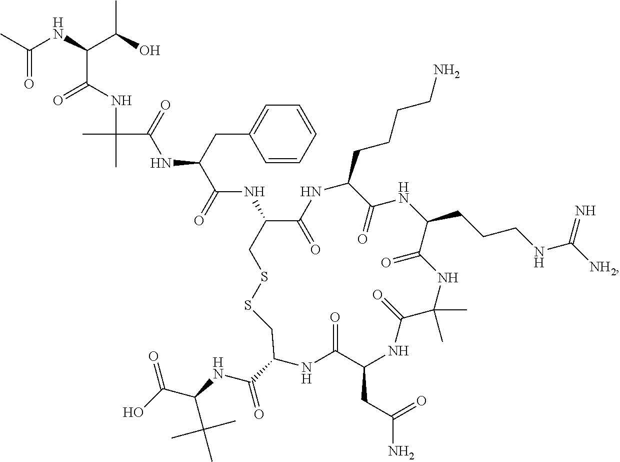 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Mcf-[Cys-Lys-Arg-Lys (DOTAGA-020c)-Asn-Cys]-Tle-Thr-NH₂ (PSM-0301) of the following formula

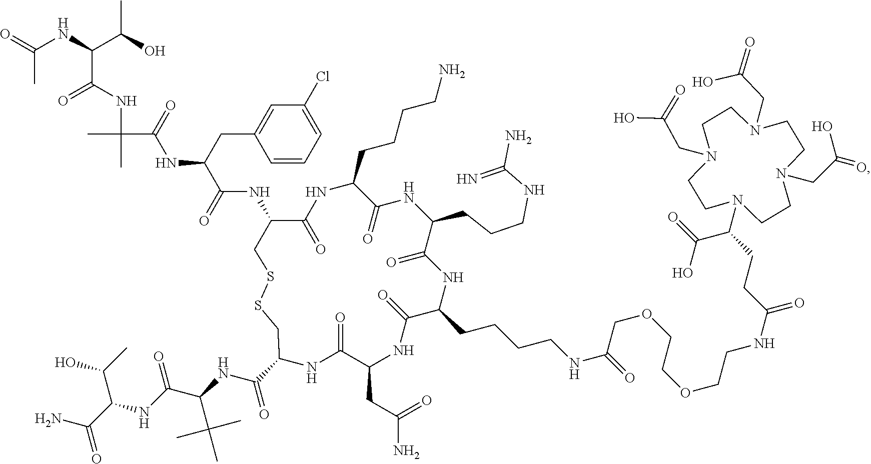 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)COCCOCCNC(=O)CC[C@H](C(=O)O)N2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Amd-Asn-Cys]-Tle-Thr-NH₂ (PSM-0302) of the following formula

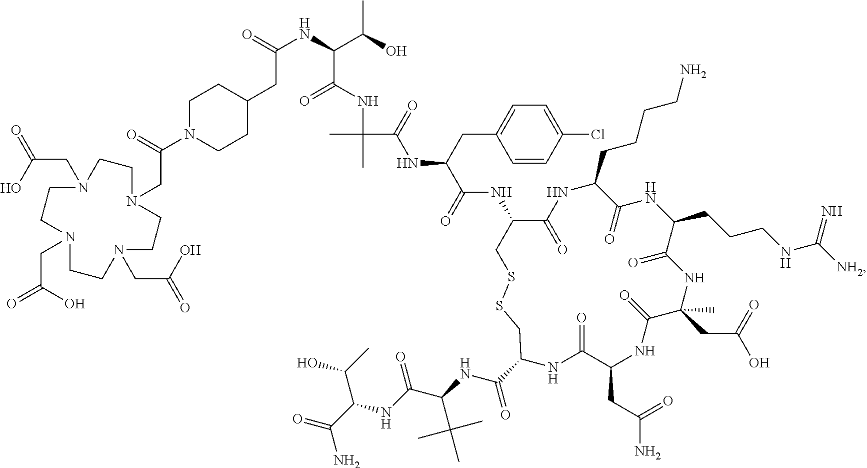 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@@](C)(CC(=O)O)C(=O)N[C@@H](CC(N)=O)C(=O)N1)C(C)(C)C)C(N)=O]

compound Ac-Thr-Aib-Phe-[Cys-Lys-Gln-Aib-Asn-Cys]-Tle-Thr-Ttds-lys(DOTA)-NH₂ (PSM-0303) of the following formula

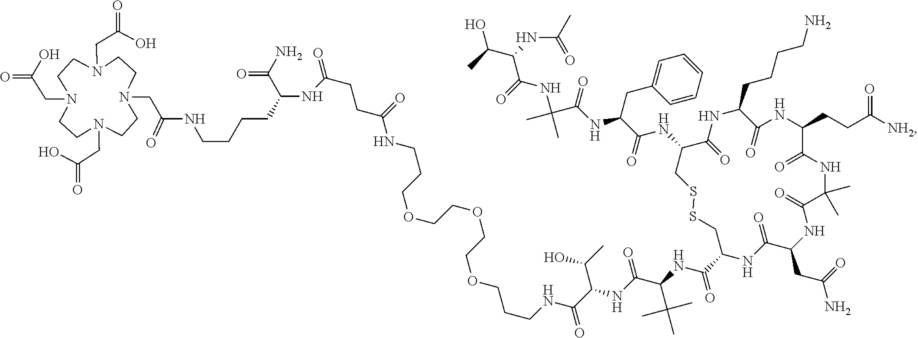 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCC(N)=O)NC(=O)[C@H](CCCCN)NC1=O)[C@@H](C)O]

compound DOTA-Cmp-Thr-Aib-6Clw-[Cys-Lys-Cit-Aib-Asn-Cys]-Tle-Thr-NH₂ (PSM-0304) of the following formula

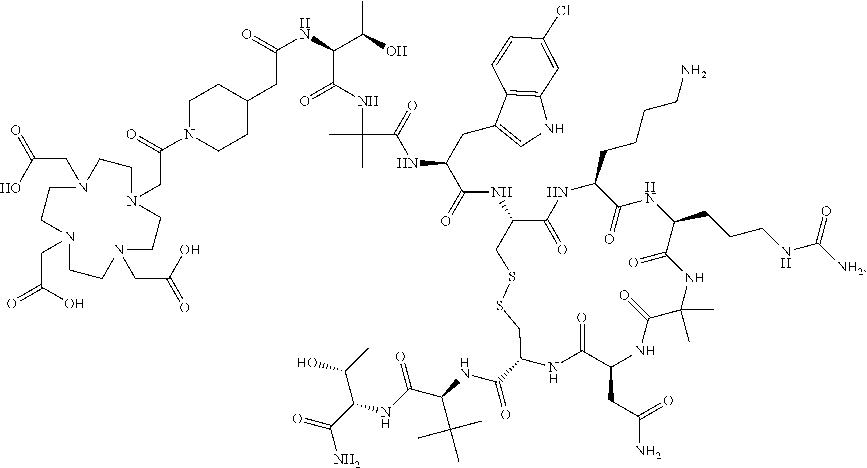 [SMILES: C[C@@H](O)[C@H](NC(=O)[C@@H](NC(=O)[C@@H]1CSSC[C@H](NC(=O)[C@H](Cc2c[nH]c3cc(Cl)ccc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Ttds-Thr-Ala-Phe-[Cys-Lys-Arg-Ala-Asn-Cys]-Lou-Thr-NH₂ (PSM-0305) of the following formula

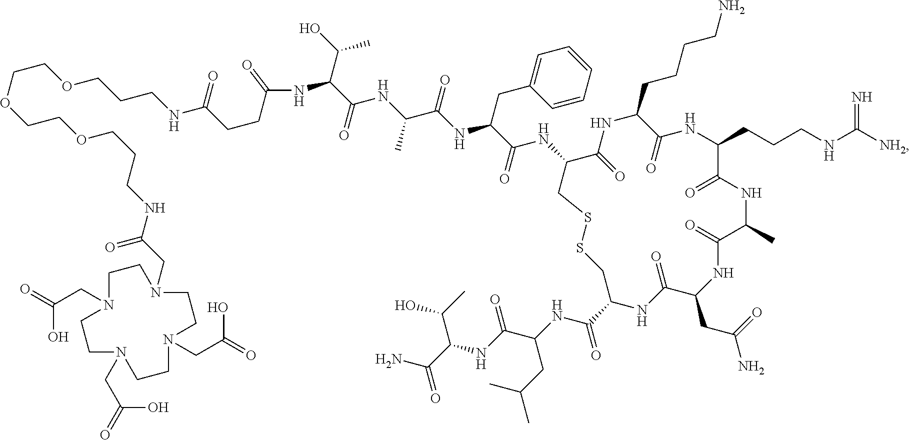 [SMILES: CC(C)CC(NC(=O)[C@@H]1CSSC[C@H](NC(=O)[C@H](Cc2ccccc2)NC(=O)[C@H](C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(N)=O)[C@@H](C)O]

compound DOTA-Cmp-Thr-Aib-Pcf-[Cys-Lys-Arg-Aib-Asn-Cys]-Tle-en (Me) 2 (PSM-0306) of the following formula

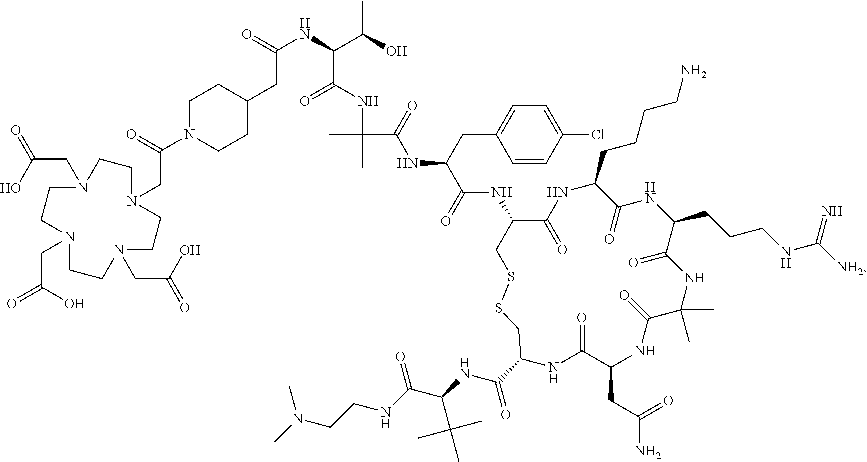 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccc(Cl)cc1)C(=O)N[C@H]1CSSC[C@@H](C(=O)N[C@H](C(=O)NCCN(C)C)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Pho-[Cys-Lys-Cit-Ala-Asn-Cys]-Tle-Thr-Asp-NH₂ (PSM-0307) of the following formula

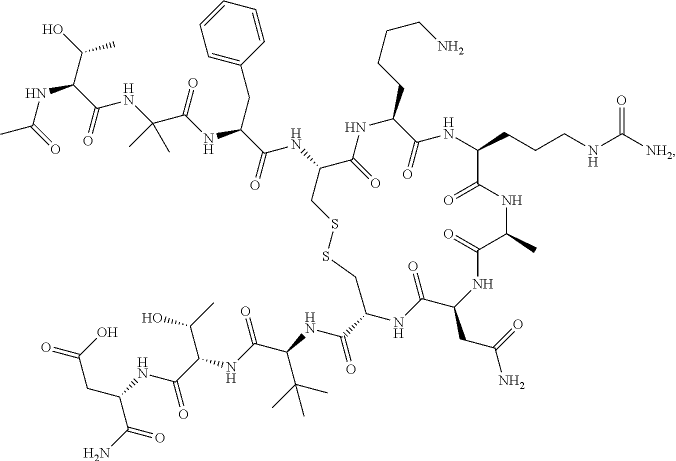 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(N)=O)NC(=O)[C@H](CCCCN)NC1=O)[C@@H](C)O]

compound DOTA-Cmp-Thr-Aib-5Clw-[Cys-Lys(Me)-Arg(Me)-Aib-Asn-Cys]-Tle-Thr-NH₂ (PSM-0308) of the following formula

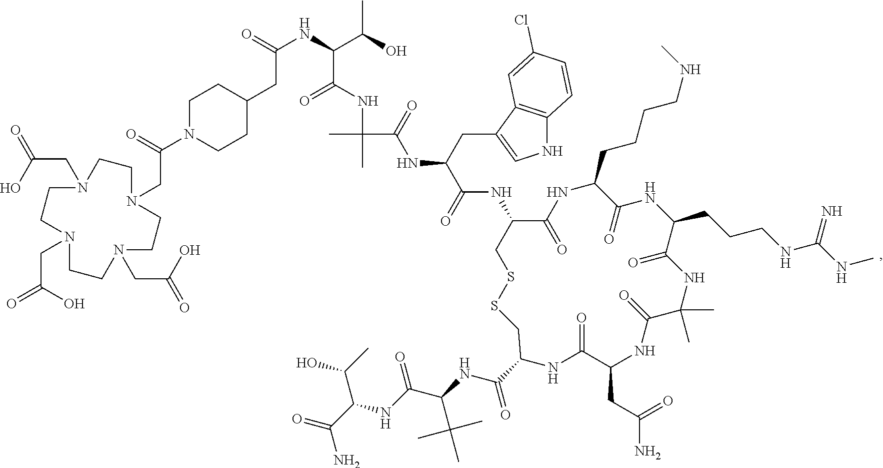 [SMILES: CNCCCC[C@@H]1NC(=O)[C@@H](NC(=O)[C@H](Cc2c[nH]c3ccc(Cl)cc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound DOTA-Ahx-Aib-Pcf-[Cys-Lys-Arg-Aib-Asn-Cys]-Tle-Thr-NH₂ (PSM-0310) of the following formula

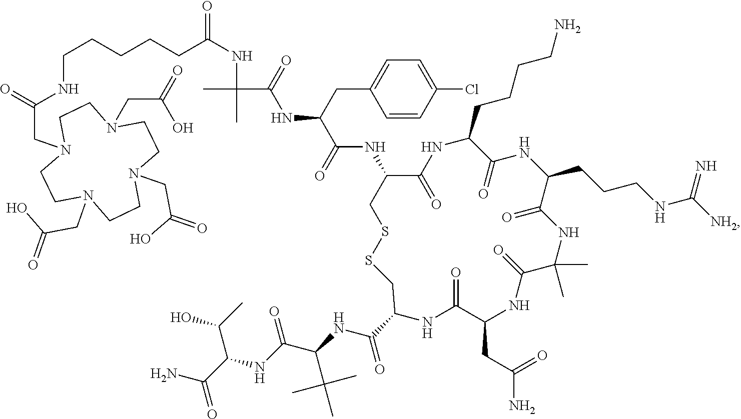 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCCCCNC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound GaDOTA-Cmp-Thr-Aib-Pcf-[Cys-Lys(Me)-Arg(Me)-Aib-Asn-Cys]-Tle-NH₂ (PSM-0311) of the following formula

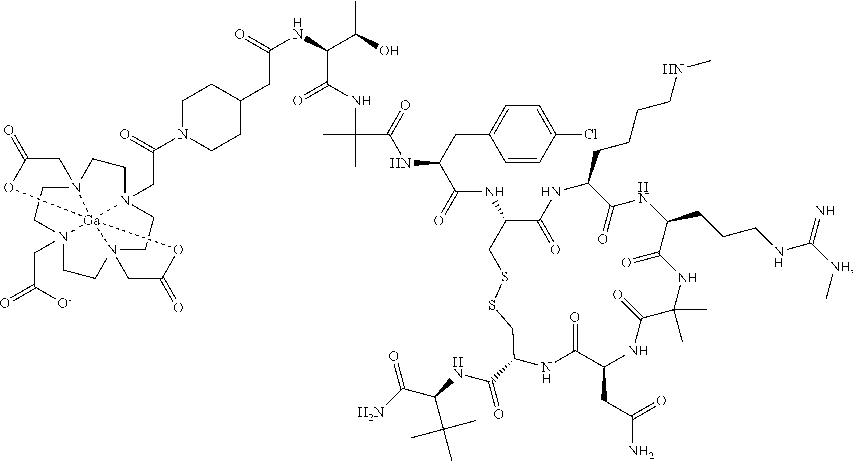

compound Hex-Thr-Aib-Phe-[Cys-Lys-Arg-Aib-Asn-Cys]-Tle-Thr-Asp-Ttds-lys (LuDOTA)-NH₂ (PSM-0312) of the following formula

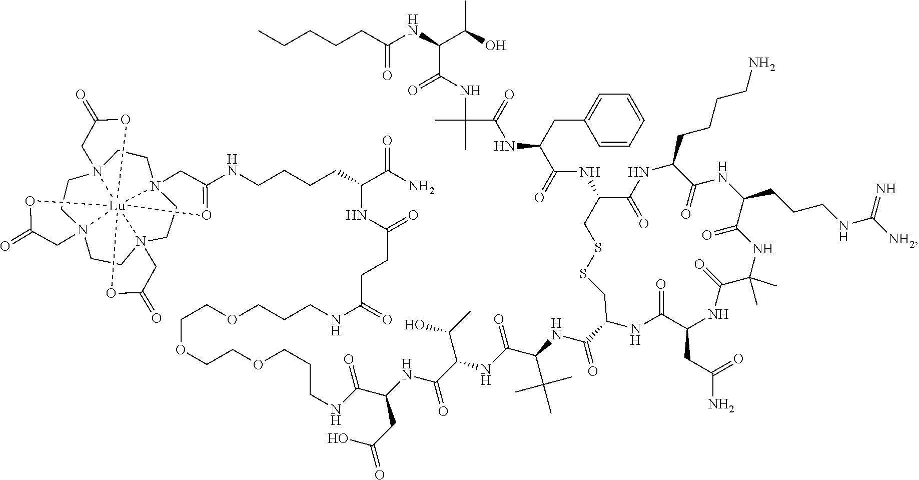

compound Ac-Thr-Ala-Phe-[Cys-Lys-Arg-Ala-Asn-Cys]-Nle-Thr-Gab-OH (PSM-0313) of the following formula

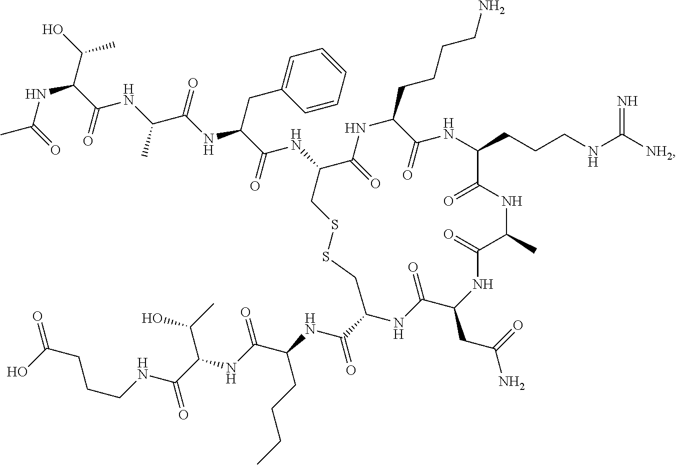 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)NCCCC(=O)O)[C@@H](C)O]

compound DOTA-Cmp-Thr-Aib-Pcf-[Cys-Lys-Arg-ams-Asn-Cys]-Tle-Thr-NH₂ (PSM-0314) of the following formula

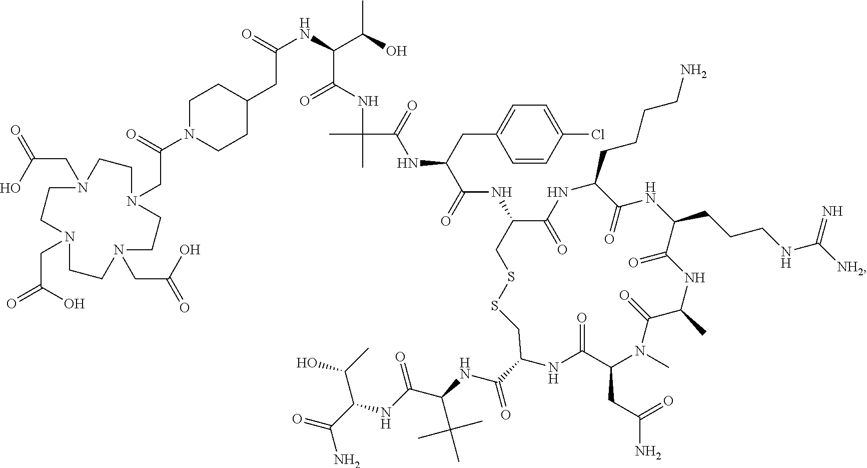 [SMILES: C[C@@H]1NC(=O)[C@H](CCCNC(=N)N)NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)N(C)C1=O]

compound Ac-Thr-Aib-Phe-[Cys-Lys-arg-Als-Asn-Cys]-Tle-Thr-Asp-NH₂ (PSM-0315) of the following formula

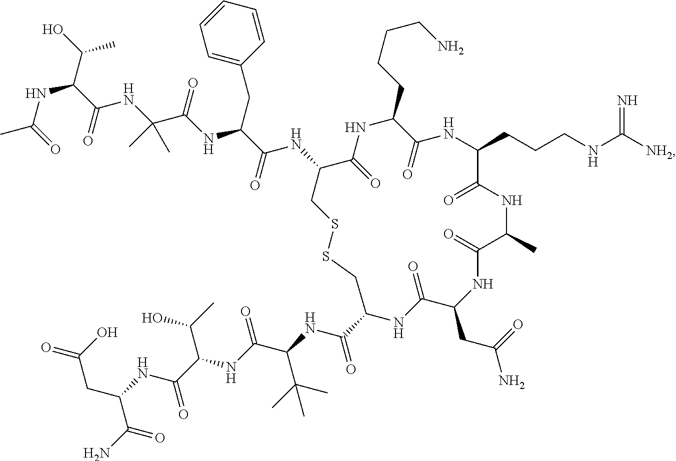 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Pab-Aib-Phe-[Cys-Lys-Arg-Aib-Asn-Cys]-Tle-Thr-NH₂ (PSM-0316) of the following formula

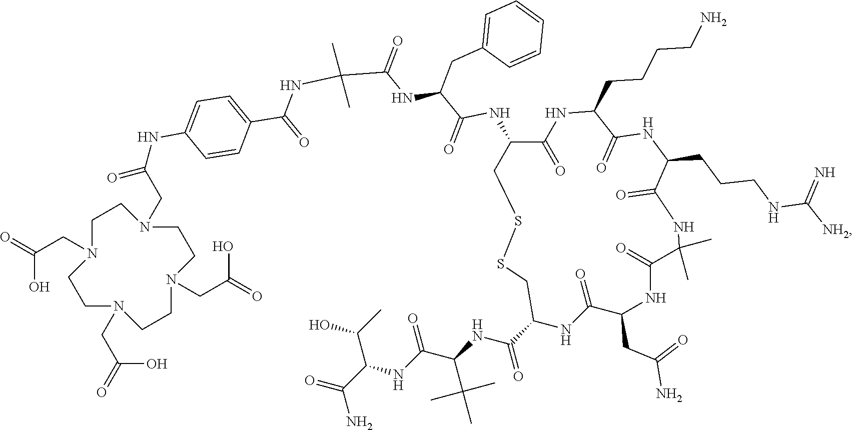 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)c2ccc(NC(=O)CN3CCN(CC(=O)O)CCN(CC(=O)O)CCN(CC(=O)O)CC3)cc2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys-Har-Aib-Asn-Cys]-Tle-Thr-NH₂ (PSM-0317) of the following formula

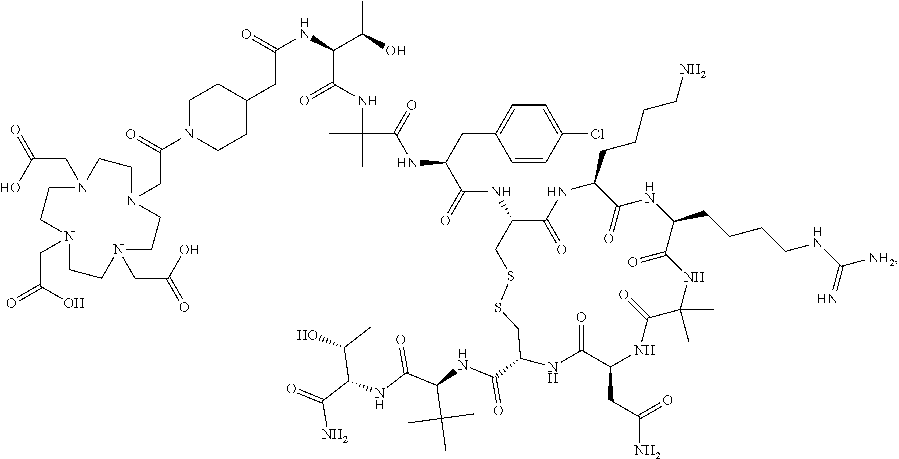 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCCNC(=N)N)C(=O)NC(C)(C)C(=O)N[C@@H](CC(N)=O)C(=O)N1)C(C)(C)C)C(N)=O]

compound Ac-Thr-Aib-Pcf-[Cys-Lys-Arg-Aib-Asn-Cys]-Tle-Thr-Asp-NH₂ (PSM-0318) of the following formula

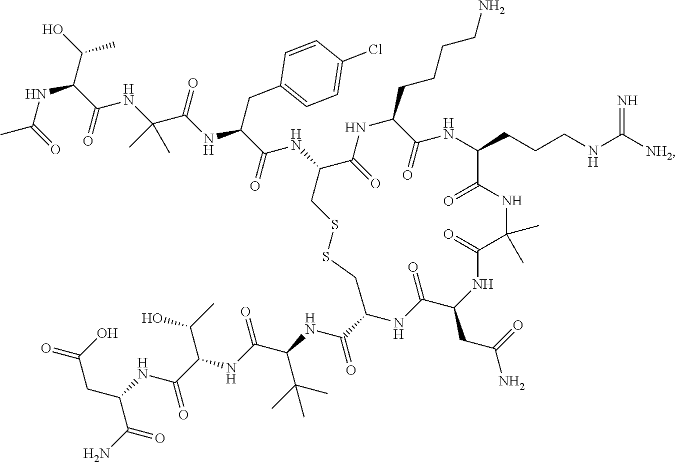 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Ala-Nmf-[Cys-Lys-Arg-Aib-Asn-Cys]-Tle-Thr-NH₂ (PSM-0319) of the following formula

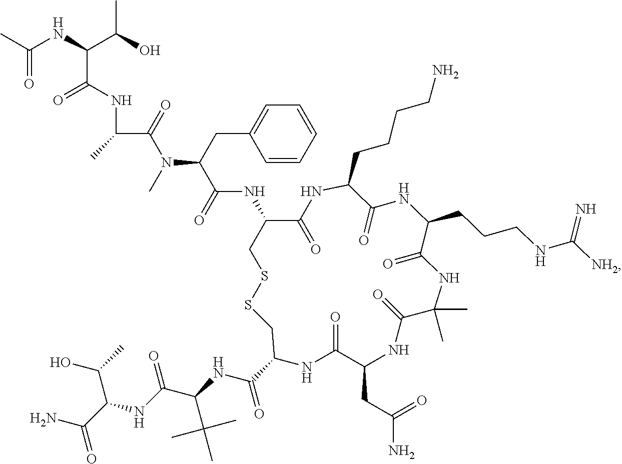 [SMILES: CC(=O)N[C@H](C(=O)N[C@@H](C)C(=O)N(C)[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Pcf-[Cys-Lys-Arg(Me)-Aib-Asn-Cys]-Tle-en-DOTA (PSM-0320) of the following formula

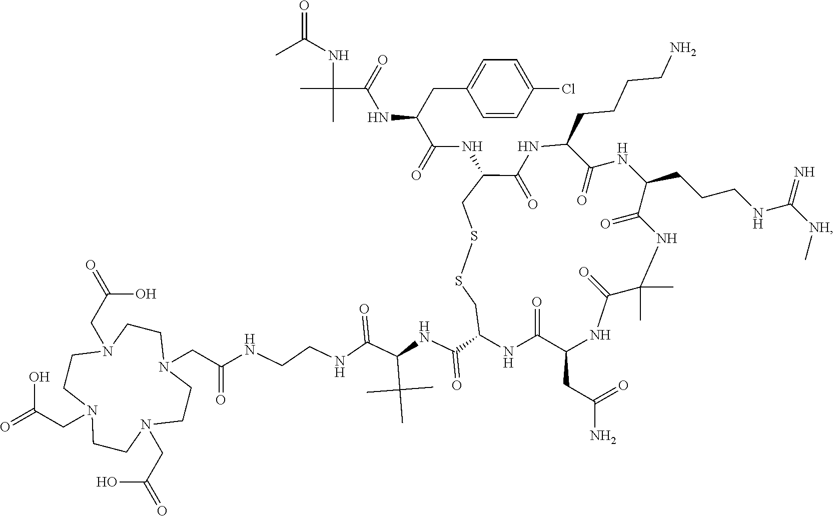 [SMILES: CNC(=N)NCCC[C@@H]1NC(=O)[C@H](CCCCN)NC(=O)[C@@H](NC(=O)[C@H](Cc2ccc(Cl)cc2)NC(=O)C(C)(C)NC(C)=O)CSSC[C@@H](C(=O)N[C@H](C(=O)NCCNC(=O)CN2CCN(CC(=O)O)CCN(CC(=O)O)CCN(CC(=O)O)CC2)C(C)(C)C)NC(=O)[C@H](CC(N)=O)NC(=O)C(C)(C)NC1=O]

compound Ac-Thr-Aib-Phe-[Cys-Lys-Arg-Apc (DOTA)-Asn-Cys]-Tle-Thr-NH₂ (PSM-0321) of the following formula

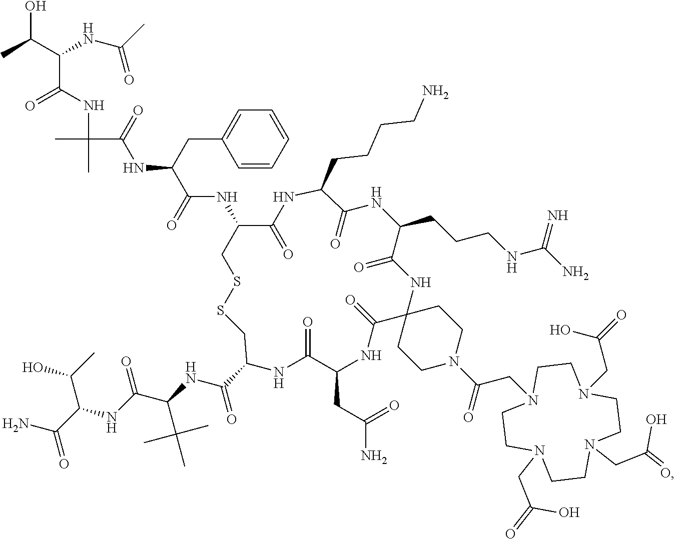 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C2(CCN(C(=O)CN3CCN(CC(=O)O)CCN(CC(=O)O)CCN(CC(=O)O)CC3)CC2)NC(=O)[C@H](CCCNC(=N)N)NC(=O)[C@H](CCCCN)NC1=O)[C@@H](C)O]

compound Ac-Thr-Aib-Pcf-[Cys-Lys-Cit-Aib-Asn-Cys]-Tle-Thr-lys (DOTA-Cmp)-NH₂ (PSM-0322) of the following formula

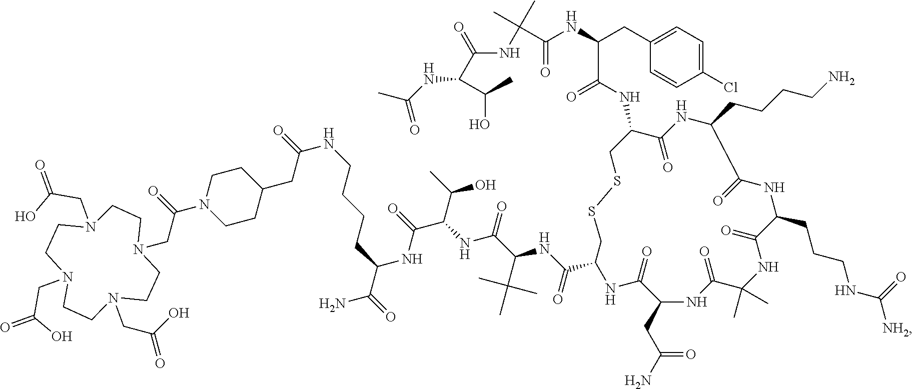 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Phe-[Cys-Lys-Arg-Ala-Asn-Cys]-Lou-Thr-Asp-Ttds-lys(DOTA)-NH₂ (PSM-0323) of the following formula

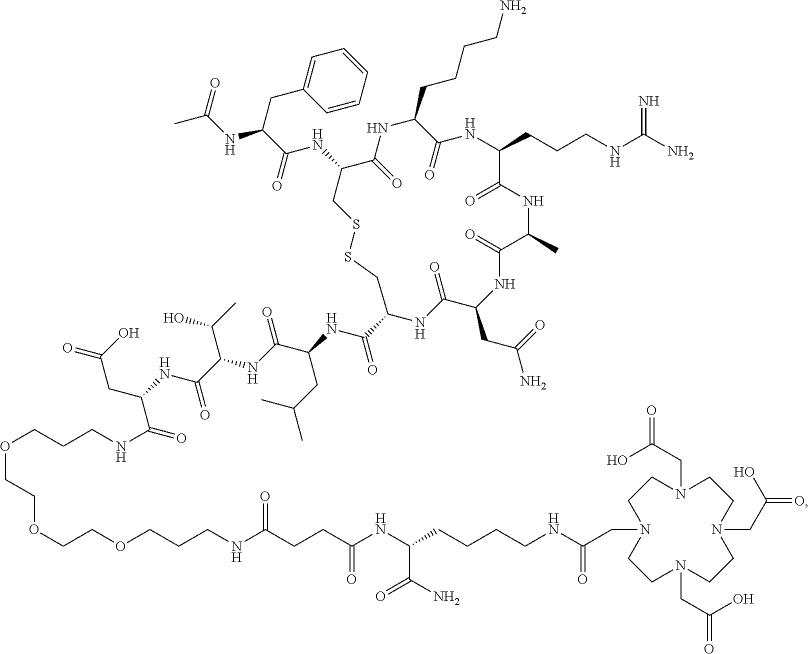 [SMILES: CC(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O]

compound HPA-Aib-Pcf-[Cys-Lys-Arg-Lys(DOTA-APAc)-Asn-Cys]-Tle-Thr-NH₂ (PSM-0324) of the following formula

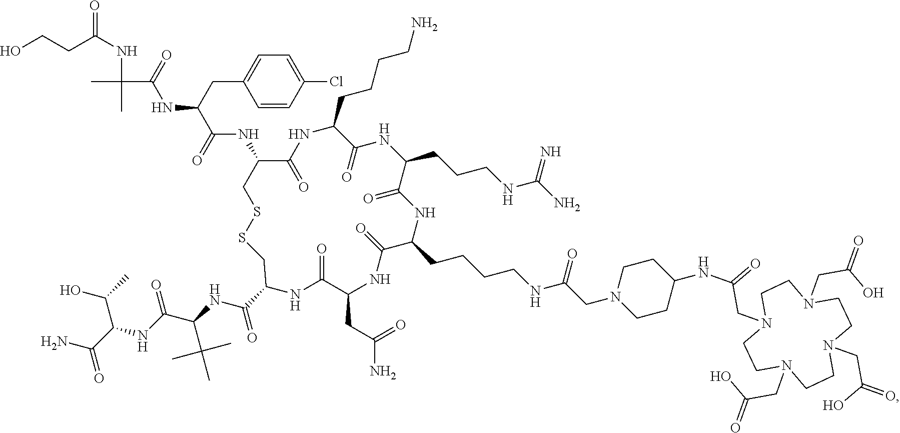 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCO)C(=O)N[C@@H](CCCCN)C(=O)N[C@@H](CCCNC(=N)N)C(=O)N[C@@H](CCCCNC(=O)CN2CCC(NC(=O)CN3CCN(CC(=O)O)CCN(CC(=O)O)CCN(CC(=O)O)CC3)CC2)C(=O)N[C@@H](CC(N)=O)C(=O)N1)C(C)(C)C)C(N)=O]

compound LuDOTA-Cmp-Thr-Aib-Pcf-[Cys-Lys-Cit-Aib-Asn-Cys]-Tle-Thr-NH₂ (PSM-0325) of the following formula

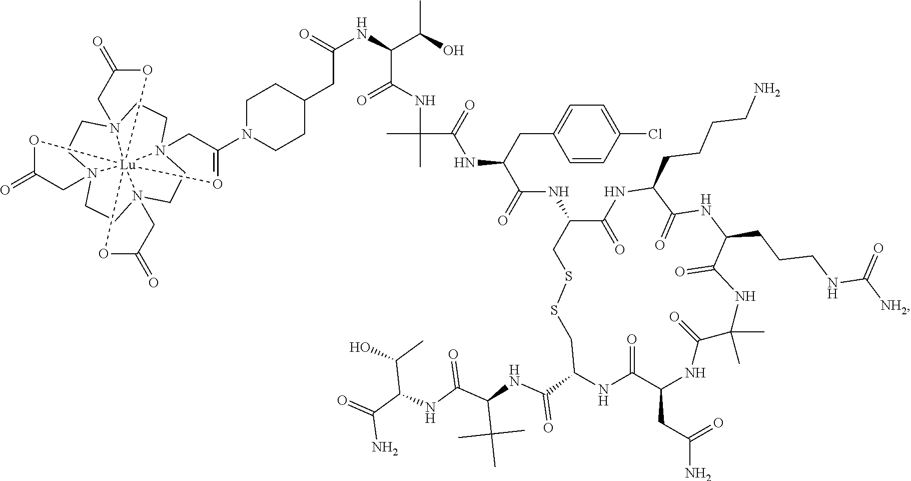

compound Ac-Thr-Aib-Phe-[Cys-Lys-Arg-Lys(DOTA)-Asn-Cys]-Tle-Thr-NH₂ (PSM-0326) of the following formula

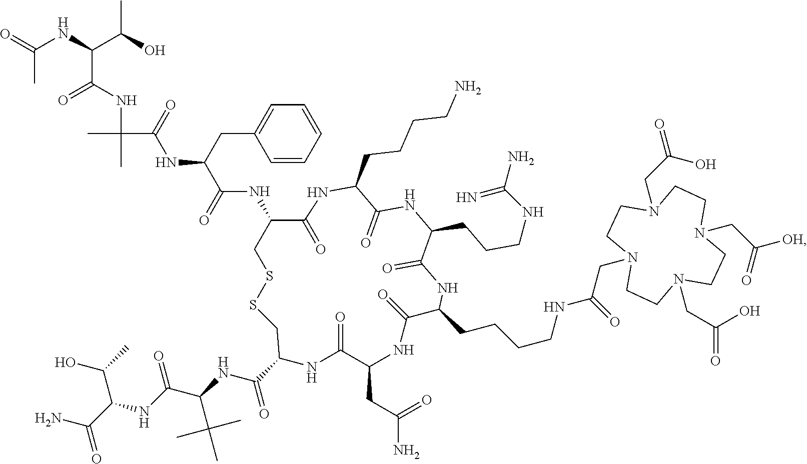 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound Ac-Thr-Aib-Phe-[cys-Lys-Arg-Aib-Asn-cys]-Tle-Thr-NH₂ (PSM-0327) of the following formula

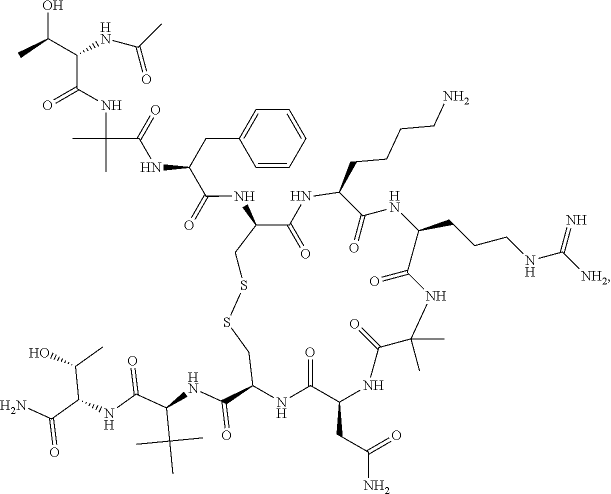 [SMILES: CC(=O)N[C@H](C(=O)NC(C)(C)C(=O)N[C@@H](Cc1ccccc1)C(=O)N[C@@H]1CSSC[C@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound SaPr-Aib-Pcf-[Cys-Lys-Arg-Aib-Asn-Cys]-Tle-Thr-lys(DOTA)-NH₂ (PSM-0328) of the following formula

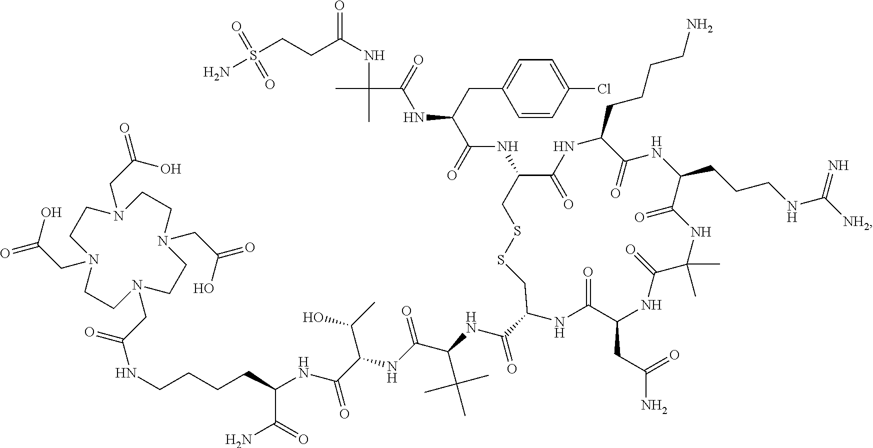 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCS(N)(=O)=O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(=O)N[C@H](CCCCNC(=O)CN1CCN(CC(=O)O)CCN(CC(=O)O)CCN(CC(=O)O)CC1)C(N)=O]

compound Ac-Thr-Aib-Pcf-[Cys-Lys-Cit-Aib-Asn-Cys]-Tle-Thr-Cmp-lys(DOTA)-NH₂ (PSM-0329) of the following formula

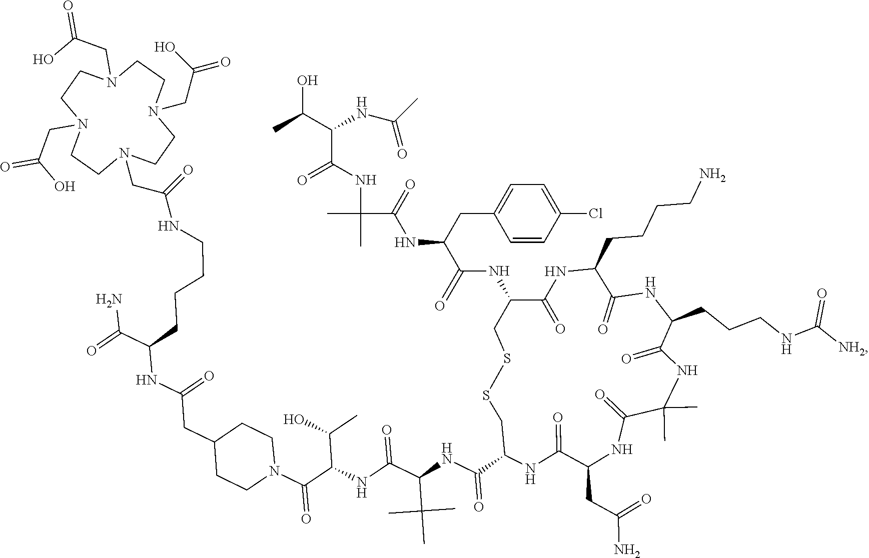 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Ala-Phe-[Cys-Lys-Arg-Ala-Asn-Cys]-Leu-Thr-Asp-Ttds-lys(DOTA)-NH₂ (PSM-0330) of the following formula

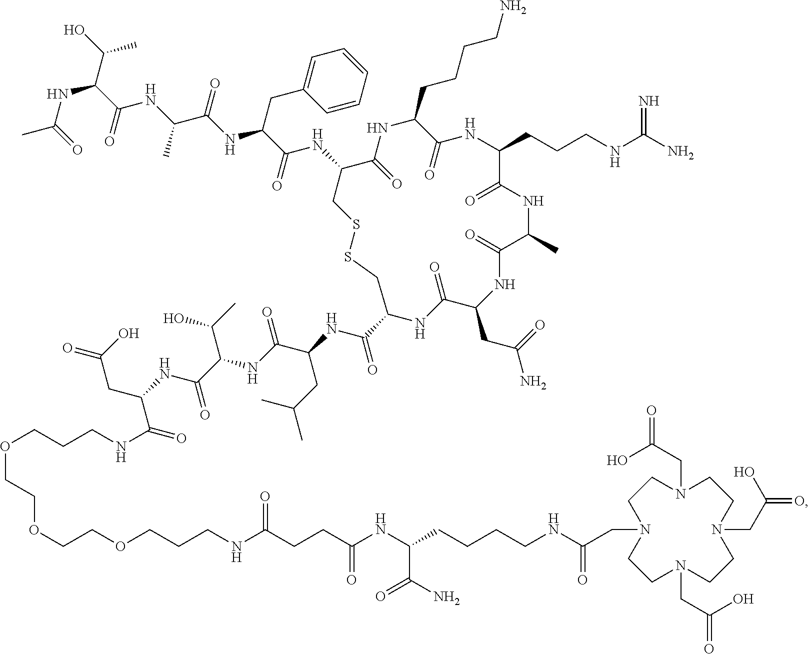 [SMILES: CC(=O)N[C@H](C(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-cys]-Tle-Thr-NH₂ (PSM-0331) of the following formula

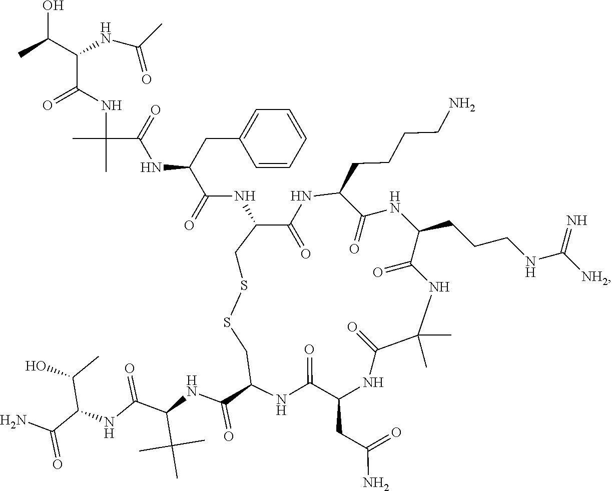 [SMILES: CC(=O)N[C@H](C(=O)NC(C)(C)C(=O)N[C@@H](Cc1ccccc1)C(=O)N[C@H]1CSSC[C@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Smc-Lys-Arg-Aib-Asn-Cys]-Tle-NH₂ (alternative: DOTA-Cmp-Thr-Aib-Pcf-[Cys-Lys-Arg-Aib-Asn-Smc]-Tle-NH₂) (PSM-0332) of the following formula

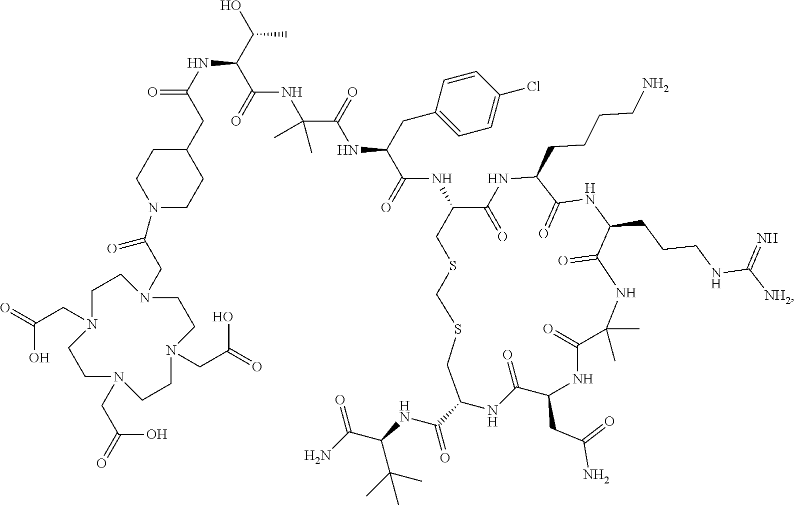 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccc(Cl)cc1)C(=O)N[C@H]1CSCSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-2Qi-[Cys-Lys-Cit-Aib-Asn-Cys]-Tle-Thr-NH₂ (PSM-0333) of the following formula

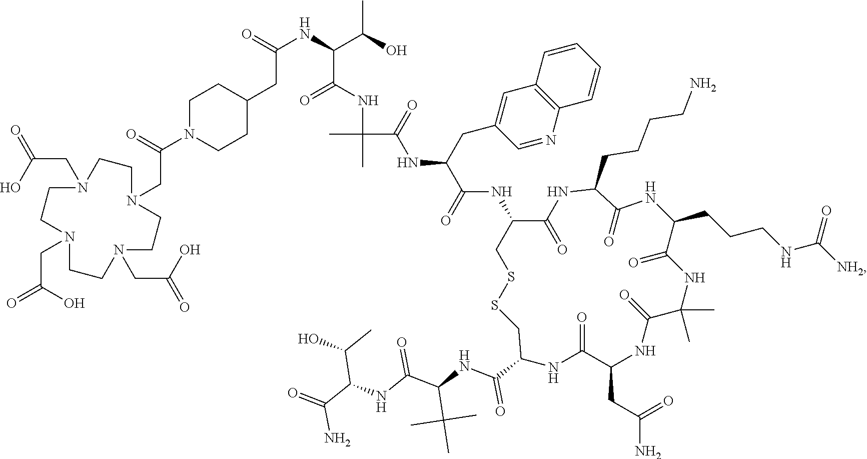 [SMILES: C[C@@H](O)[C@H](NC(=O)[C@@H](NC(=O)[C@@H]1CSSC[C@H](NC(=O)[C@H](Cc2cnc3ccccc3c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound Ac-Thr-Aib-Phe-[Cys-Lys-Gln-Aib-Asn-Cys]-Tle-Thr-Asp-NH₂ (PSM-0334) of the following formula

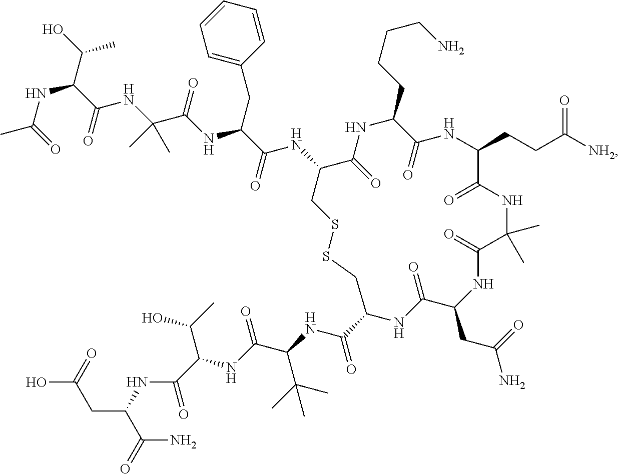 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCC(N)=O)NC(=O)[C@H](CCCCN)NC1=O)[C@@H](C)O]

compound Ac-Thr-Ala-Phe-[Cys-Lys-Arg-Ala-Ala-Cys]-Nle-Thr-Asp-NH₂ (PSM-0335) of the following formula

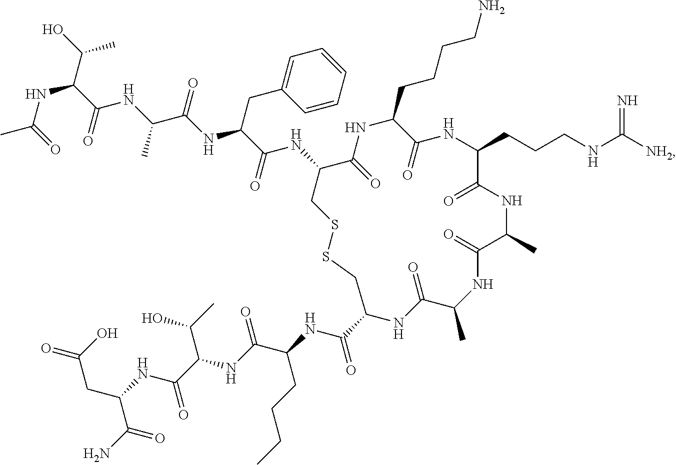 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](C)C(=O)N1)C(=O)N[C@H](C(=O)N[C@@H](CC(=O)O)C(N)=O)[C@@H](C)O]

compound Ac-Thr-Aib-Pcf-[Cys-Lys-Arg-Aib-Asn-Cys]-Tle-Thr-NH₂ (PSM-0336) of the following formula

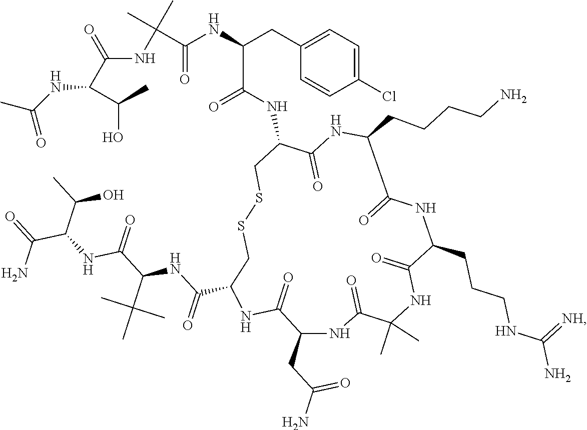 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-2Qi-[Cys-Lys(Me)-Arg(Me)-Aib-Asn-Cys]-Tle-Thr-NH₂ (PSM-0337) of the following formula

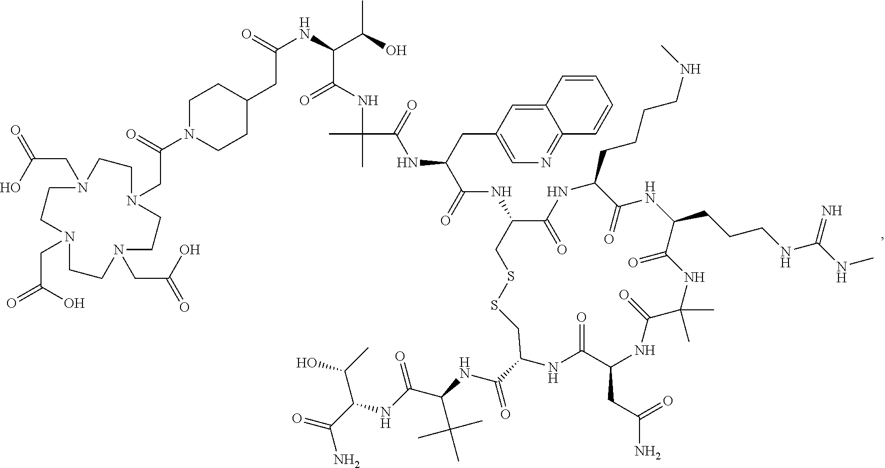 [SMILES: CNCCCC[C@@H]1NC(=O)[C@@H](NC(=O)[C@H](Cc2cnc3ccccc3c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound Ac-Thr-Aib-Pcf-[Cys-Lys-Nle-Aib-Asn-Cys]-Tle-Thr-NH₂ (PSM-0338) of the following formula

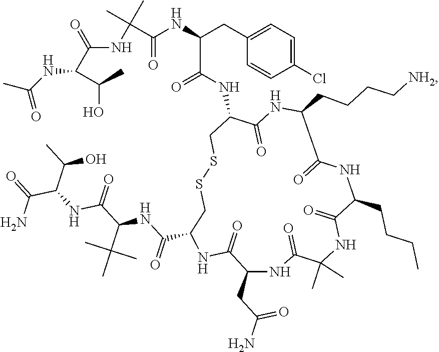 [SMILES: CCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound Ac-Thr-Aib-Phe-[Cys-Lys-Arg-Aib-Asn-Cys]-Tle-Thr-Cmp-lys (DOTAGA)-NH₂ (PSM-0339) of the following formula

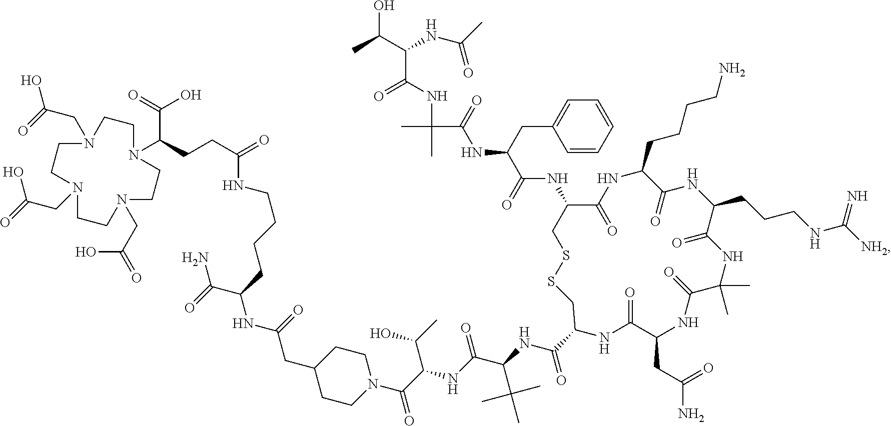 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CC[C@H](C(=O)O)N3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Nmr-Aib-Asn-Cys]-Tle-Thr-NH₂ (PSM-0340) of the following formula

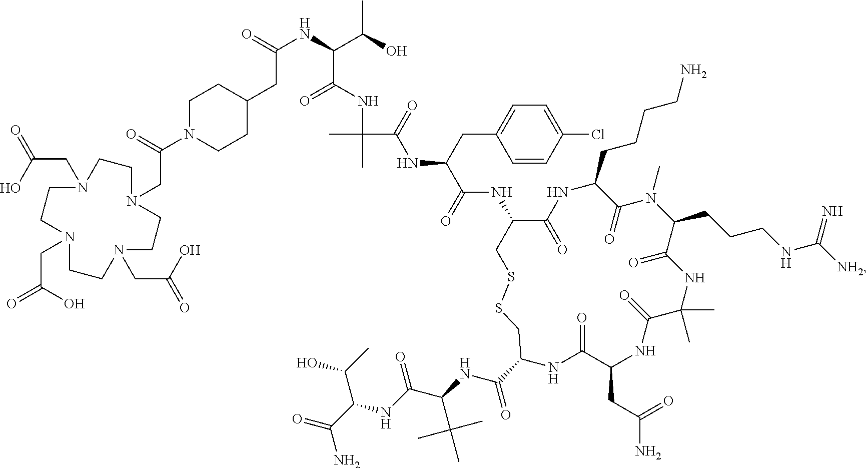 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N(C)[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys-Lys(Ac)-Aib-Asn-Cys]-Tle-Thr-NH₂ (PSM-0341) of the following formula

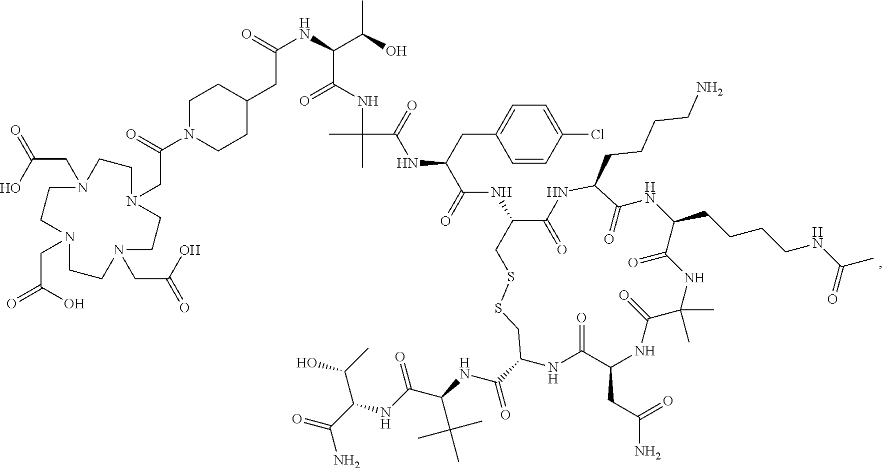 [SMILES: CC(=O)NCCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound DOTA-Ttds-Thr-Ala-Phe-[Cys-Lys-Arg-Ala-Asn-Cys]-Tle-Thr-Asp-NH₂ (PSM-0342) of the following formula

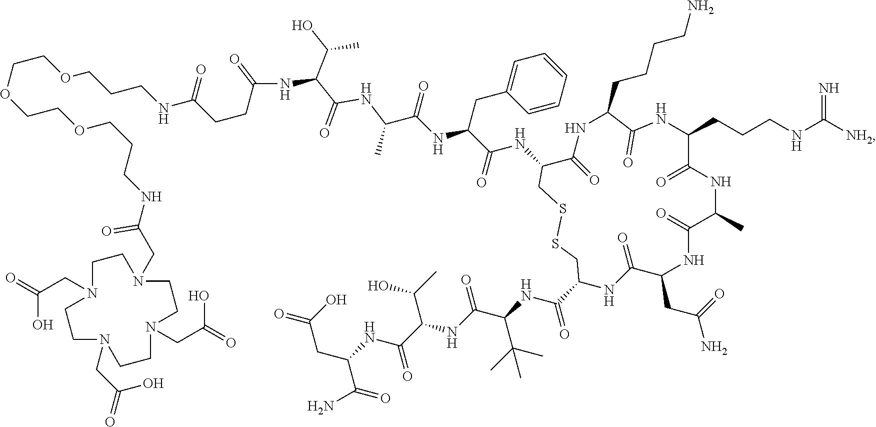 [SMILES: C[C@@H]1NC(=O)[C@H](CCCNC(=N)N)NC(=O)[C@H](CCCCN)NC(=O)[C@@H](NC(=O)[C@H](Cc2ccccc2)NC(=O)[C@H](C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC1=O]

compound DOTA-Cmp-Thr-glu-Pcf-[Cys-Lys-Arg-Aib-Asn-Cys]-Tle-Thr-NH₂ (PSM-0343) of the following formula

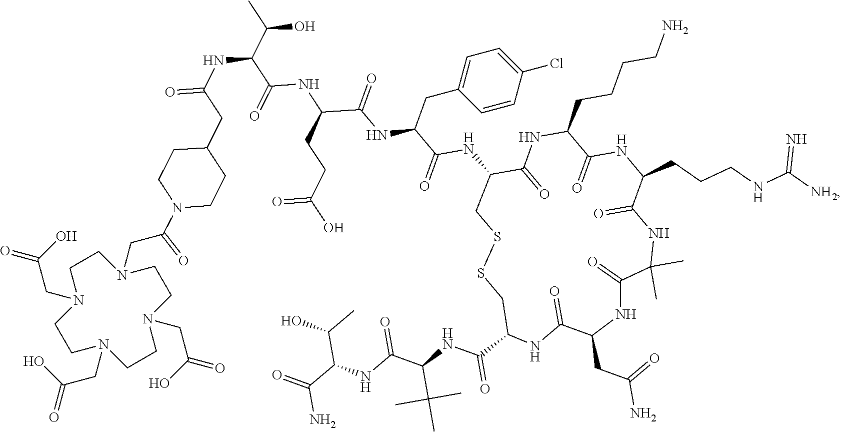 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@@H](CCC(=O)O)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys-Arg(Me)-Aib-Asn-Cys]-Tle-NH₂ (PSM-0345) of the following formula

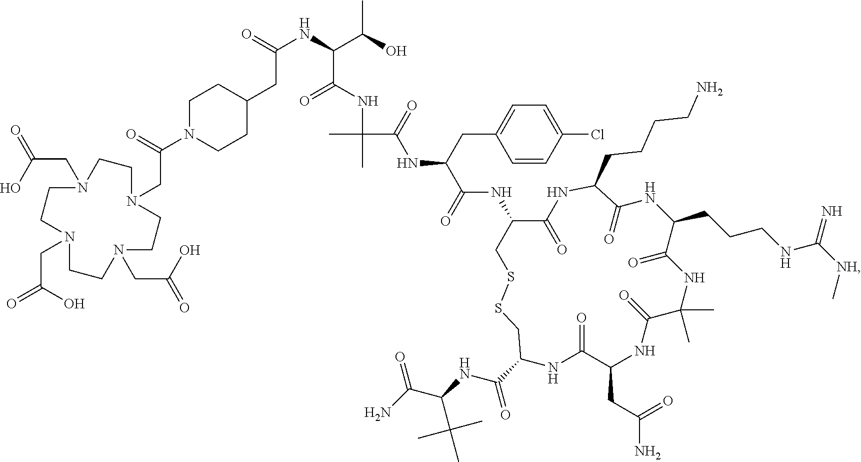 [SMILES: CNC(=N)NCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC1=O]

compound DOTA-Cmp-Thr-Amd-Pcf-[Cys-Lys-Arg-Aib-Asn-Cys]-Tle-Thr-NH₂ (PSM-0346) of the following formula

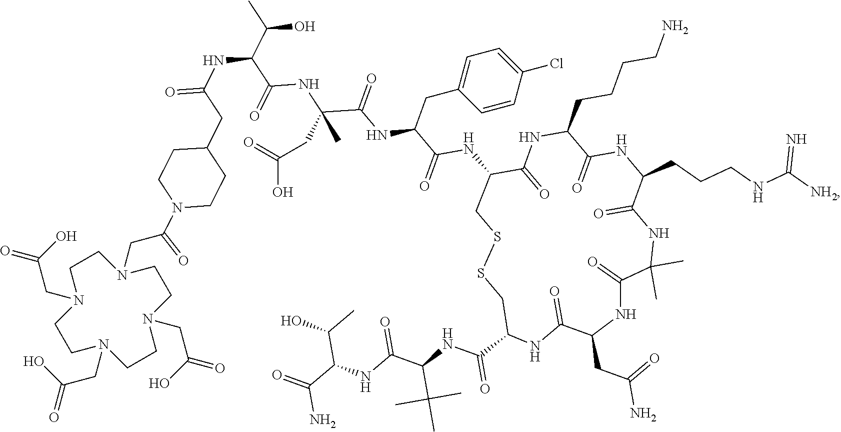 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@](C)(CC(=O)O)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Cys-Lys-Arg-Aib-Asn-Cys]-Tle-Thr-Cmp-lys (LuDOTA)-NH₂ (PSM-0347) of the following formula

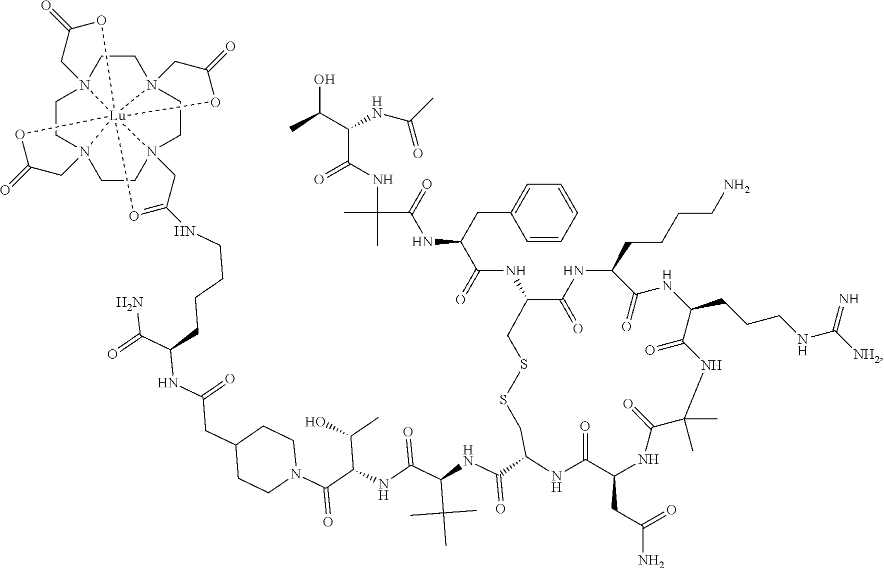

compound Ac-Thr-Aib-Phe-[cys-Lys-Arg-Aib-Asn-Cys]-Tle-Thr-NH₂ (PSM-0348) of the following formula

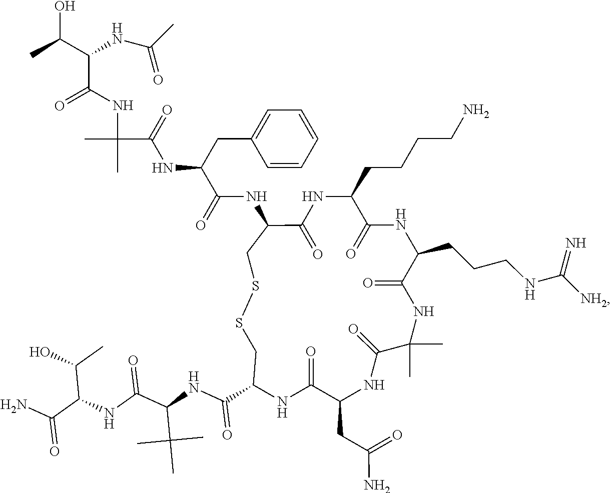 [SMILES: CC(=O)N[C@H](C(=O)NC(C)(C)C(=O)N[C@@H](Cc1ccccc1)C(=O)N[C@@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Pcf-[Cys-Lys-Arg-Aib-Asn-Cys]-Tle-Thr-Cmp-lys(DOTA)-NH₂ (PSM-0349) of the following formula

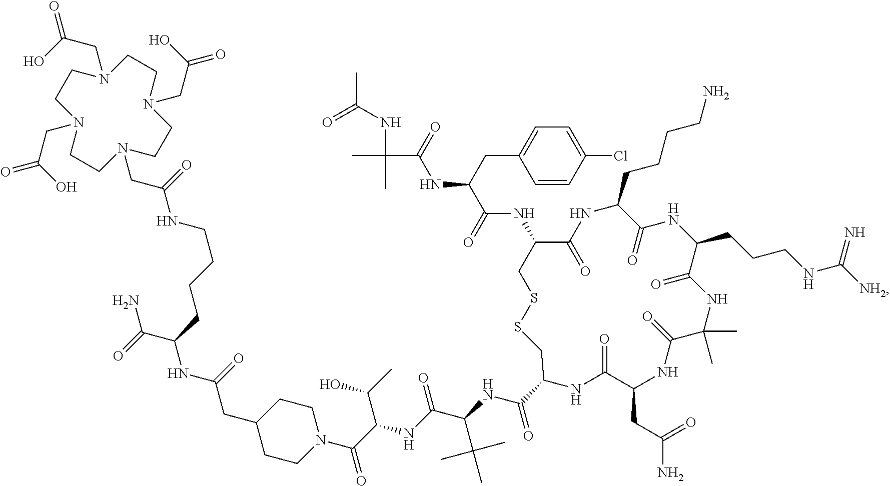 [SMILES: CC(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Lys-Arg-Aib-Asn-Cys]-leu-Thr-NH₂ (PSM-0350) of the following formula

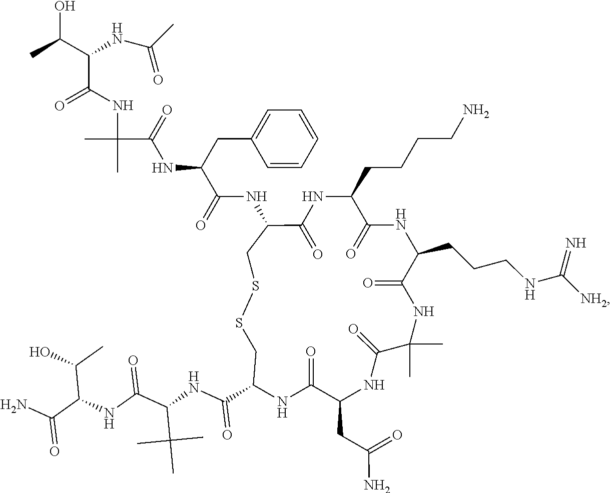 [SMILES: CC(=O)N[C@H](C(=O)NC(C)(C)C(=O)N[C@@H](Cc1ccccc1)C(=O)N[C@H]1CSSC[C@@H](C(=O)N[C@@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg(Me)-Aib-Asn-Cys]-Tle-Thr-NH₂ (PSM-0351) of the following formula

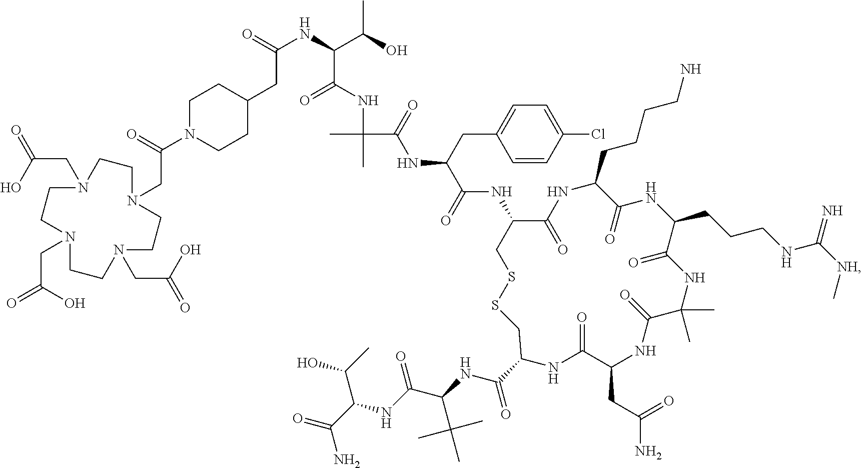 [SMILES: CNC(=N)NCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound DOTA-Bal-Aib-Pcf-[Cys-Lys-Arg-Aib-Asn-Cys]-Tle-Thr-NH₂ (PSM-0352) of the following formula

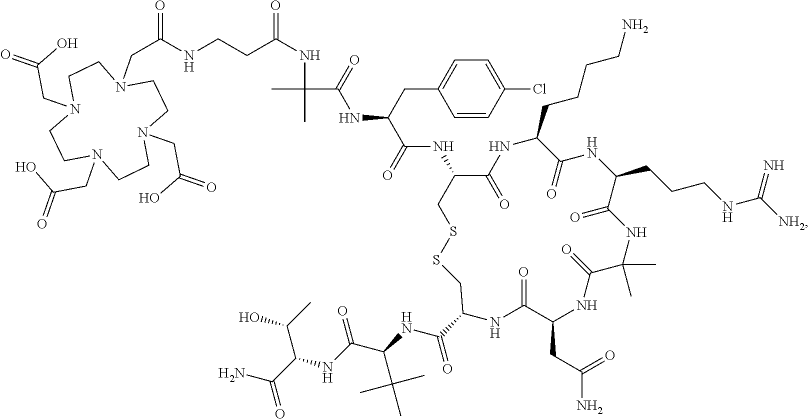 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCNC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Iva-Aib-Pcf-[Cys-Lys-Arg-Lys(DOTA-APAc)-Asn-Cys]-Tle-Thr-NH₂ (PSM-0353) of the following formula

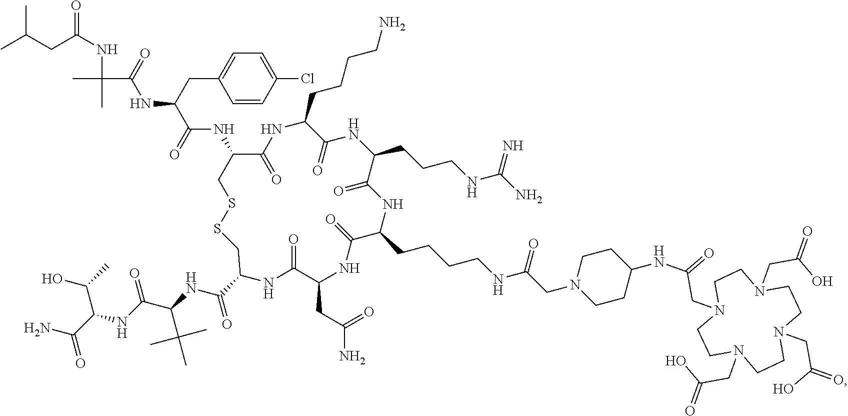 [SMILES: CC(C)CC(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CN2CCC(NC(=O)CN3CCN(CC(=O)O)CCN(CC(=O)O)CCN(CC(=O)O)CC3)CC2)NC(=O)[C@H](CCCNC(=N)N)NC(=O)[C@H](CCCCN)NC1=O]

compound DOTA-Cmp-Thr-Aib-Mcf-[Cys-Lys(Me)-Arg(Me)-Aib-Asn-Cys]-Tle-NH₂ (PSM-0354) of the following formula

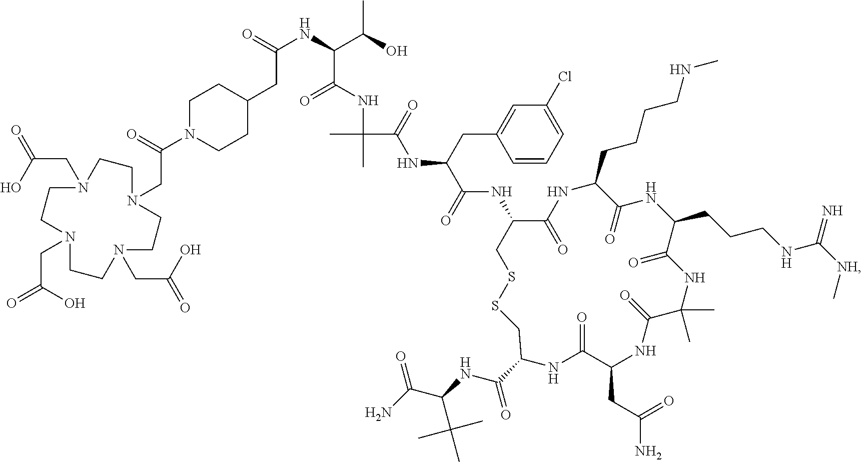 [SMILES: CNCCCC[C@@H]1NC(=O)[C@@H](NC(=O)[C@H](Cc2cccc(Cl)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound DOTA-Cmp-Thr-Aib-Pcf-[Cys-Lys(Me)-RMe3-Aib-Asn-Cys]-Tle-Thr-NH₂ (PSM-0355) of the following formula

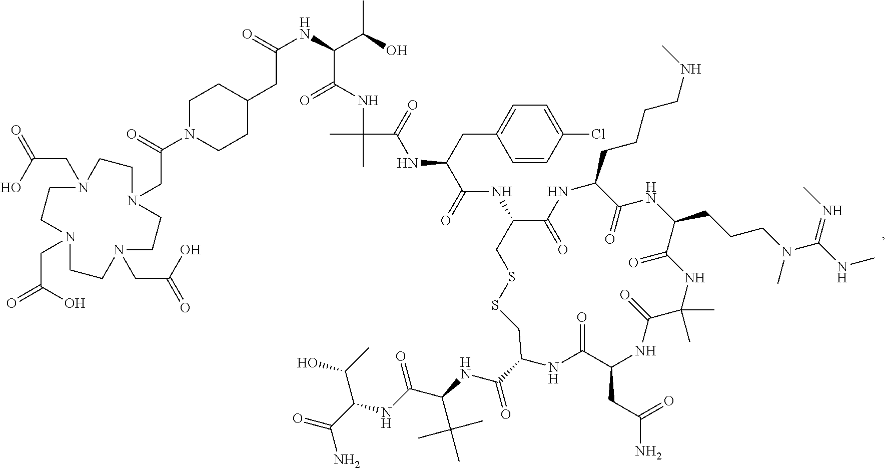 [SMILES: C/N=C(/NC)N(C)CCC[C@@H]1NC(=O)[C@H](CCCCNC)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound GaDOTA-Cmp-Thr-Aib-Pcf-[Cys-Lys(Me)-Arg(Me)-Aib-Asn-Cys]-Tle-Thr-NH₂ (PSM-0356) of the following formula

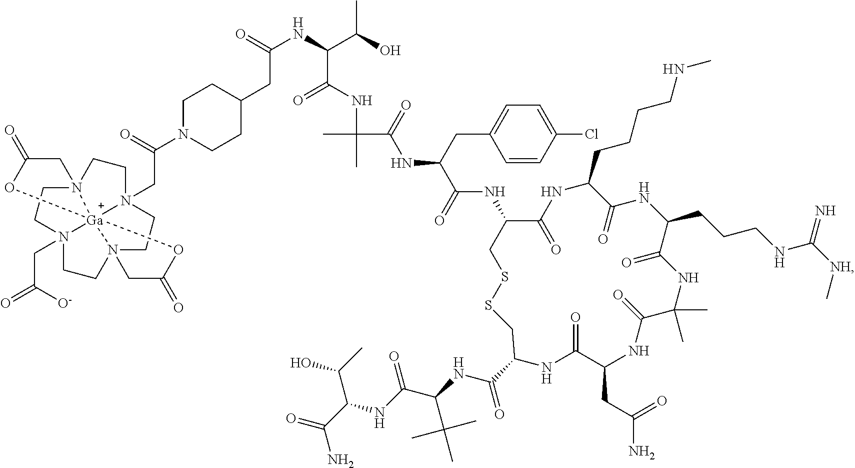

compound DOTA-Cmp-Ile-Aib-Phe-[Cys-Lys-Arg-Aib-Asn-Cys]-Tle-Thr-NH₂ (PSM-0357) of the following formula

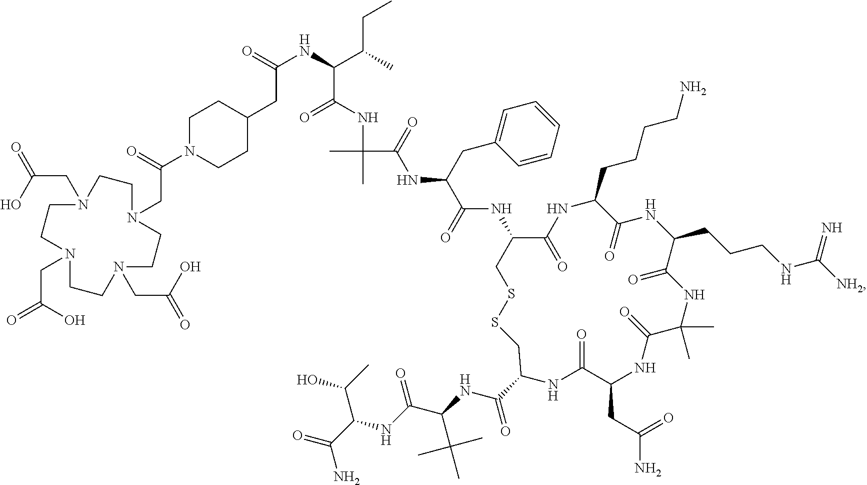 [SMILES: CC[C@H](C)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Lys-Arg-Lys (InDOTA-Ttds)-Asn-Cys]-Tle-Thr-Asp-NH₂ (PSM-0358) of the following formula

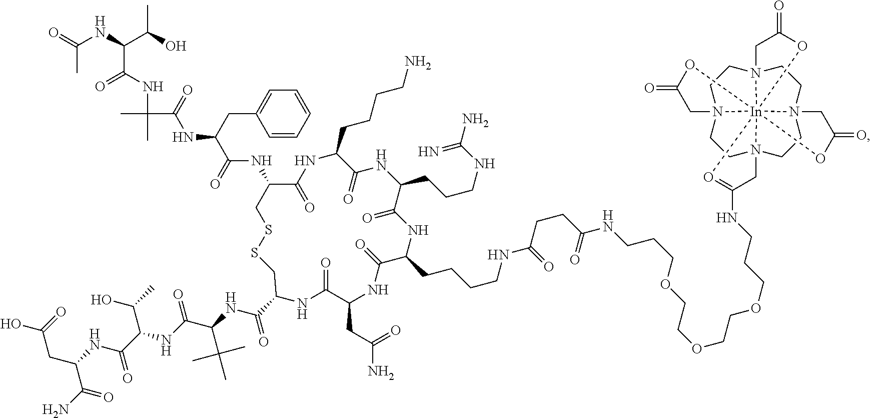

compound DOTA-Cmp-Thr-Aib-5Brw-[Cys-Lys-Cit-Aib-Asn-Cys]-Tle-Thr-NH₂ (PSM-0359) of the following formula

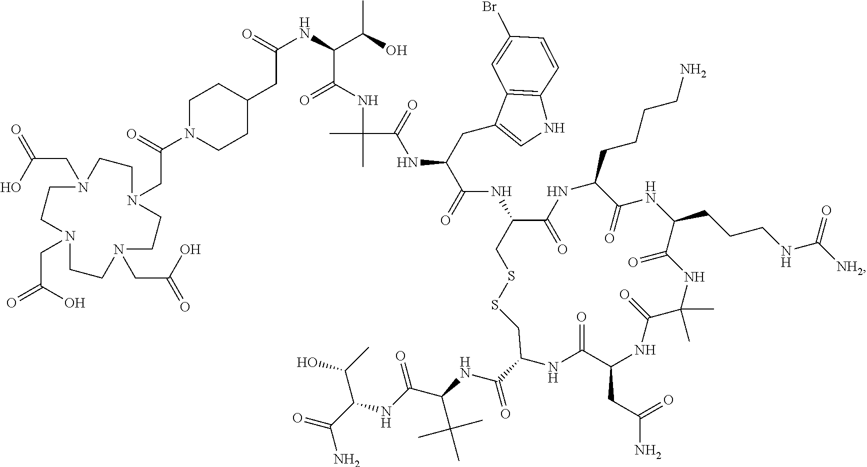 [SMILES: C[C@@H](O)[C@H](NC(=O)[C@@H](NC(=O)[C@@H]1CSSC[C@H](NC(=O)[C@H](Cc2c[nH]c3ccc(Br)cc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Cmp-Thr-Aib-Phe-[Cys-Lys-Arg-Aib-Asn-Cys]-Tle-NH₂ (PSM-0361) of the following formula

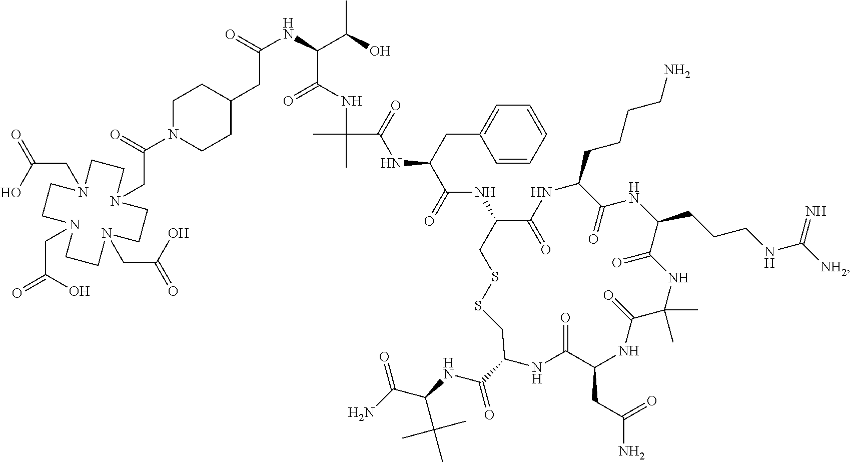 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccccc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound InDOTA-Cmp-Thr-Aib-Pcf-[Cys-Lys-Arg-Aib-Asn-Cys]-Tle-Thr-NH₂ (PSM-0362) of the following formula

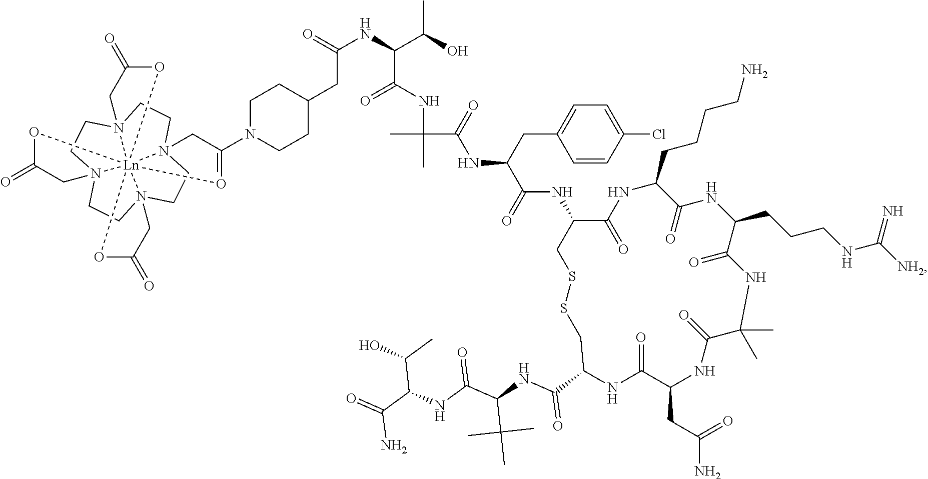

compound Ac-Thr-Aib-Phe-[Cys-Lys-Glu-Ala-Asn-Cys]-Tle-Thr-Asp-NH₂ (PSM-0363) of the following formula

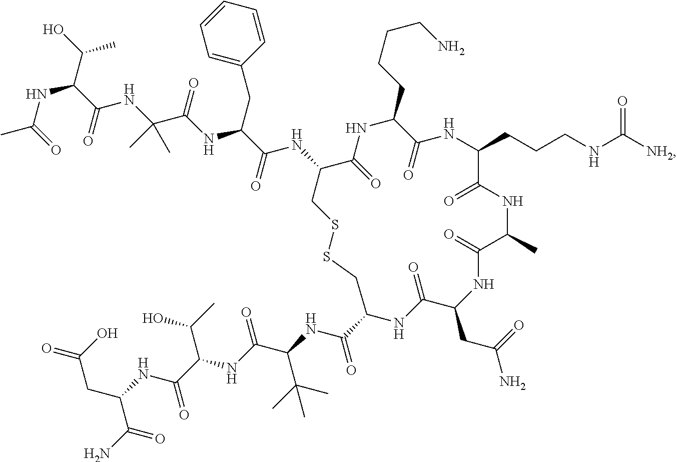 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(N)=O)NC(=O)[C@H](CCCCN)NC1=O)[C@@H](C)O]

compound DOTA-Cmp-Thr-Aib-Pcf-[Cys-Lys-Cit-Aib-Asn-Cys]-Tle-Thr-NH₂ (PSM-0365) of the following formula

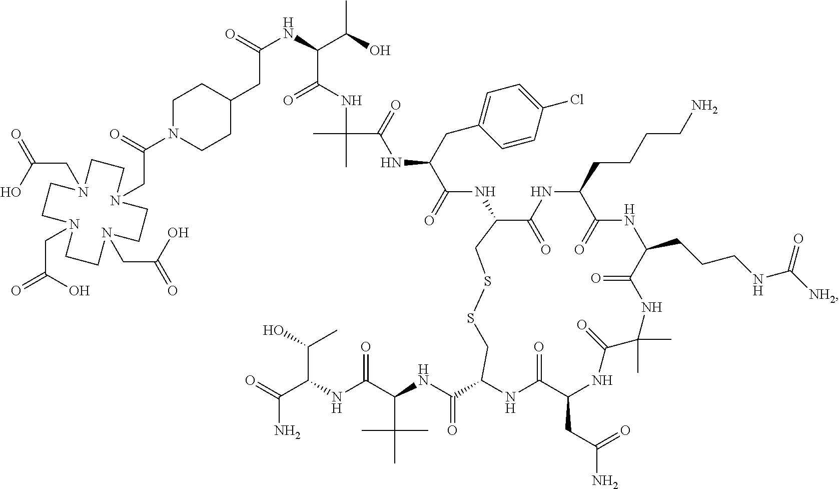 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Cmp-Thr-amd-Pcf-[Cys-Lys-Arg-Aib-Asn-Cys]-Tle-Thr-NH₂ (PSM-0366) of the following formula

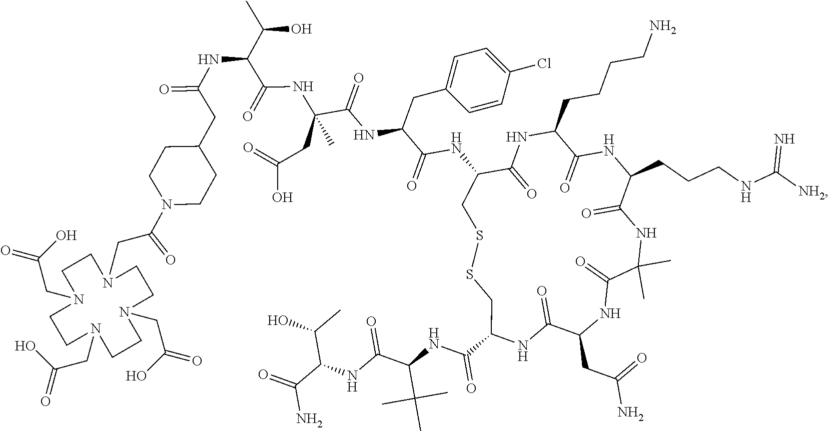

compound Ac-Thr-Aib-Pcf-[Cys-Lys-Arg-Aib-Asn-Cys]-Tle-NH₂ (PSM-0367) of the following formula

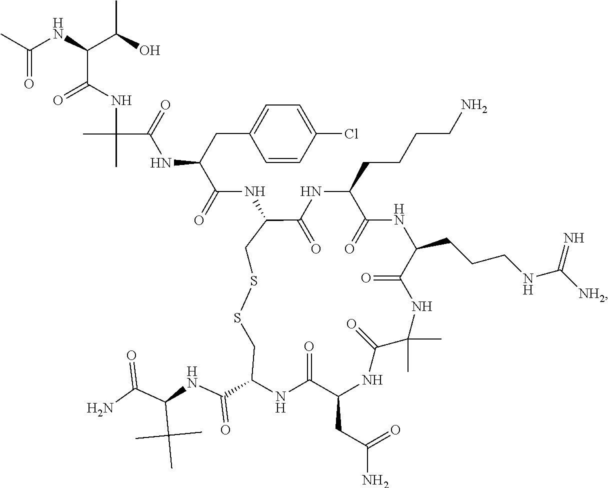 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Ahx-Thr-Aib-Phe-[Cys-Lys-Arg-Aib-Ass-Cys]-Tle-Thr-NH₂ (PSM-0368) of the following formula

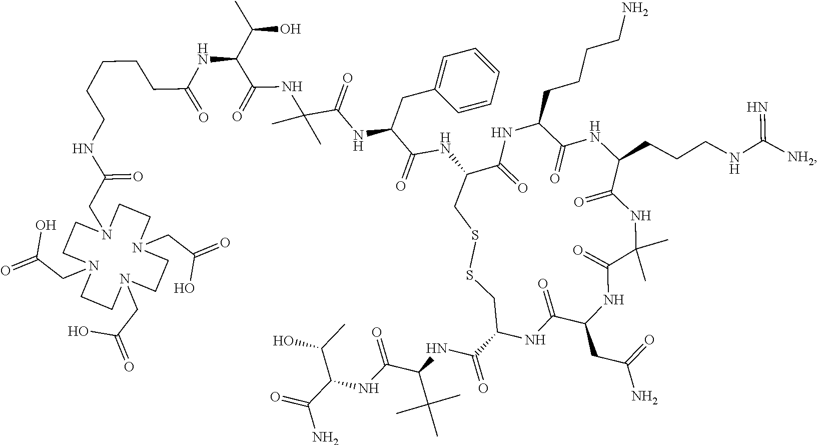 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CCCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Ams-Pcf-[Cys-Lys-Arg-Aib-Asn-Cys]-Tle-Thr-NH₂ (PSM-0369) of the following formula

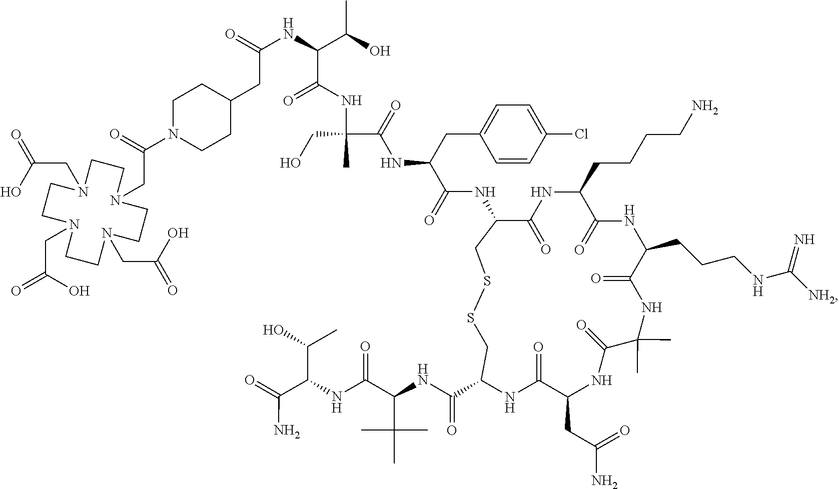 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@](C)(CO)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Pcf-[Cys-Lys-Arg-Lys(DOTA-020c)-Asn-Cys]-Tle-Thr-NH₂ (PSM-0370) of the following formula

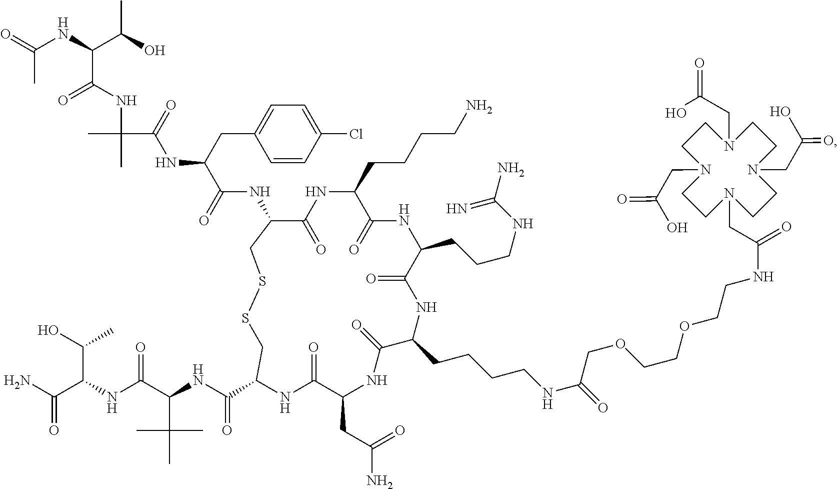 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)COCCOCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound nBuCAyl-Thr-Aib-Pcf-[Cys-Lys-Arg-Aib-Asn-Cys]-Tle-Thr-Cmp-lys(DOTA)-NH₂ (PSM-0371) of the following formula

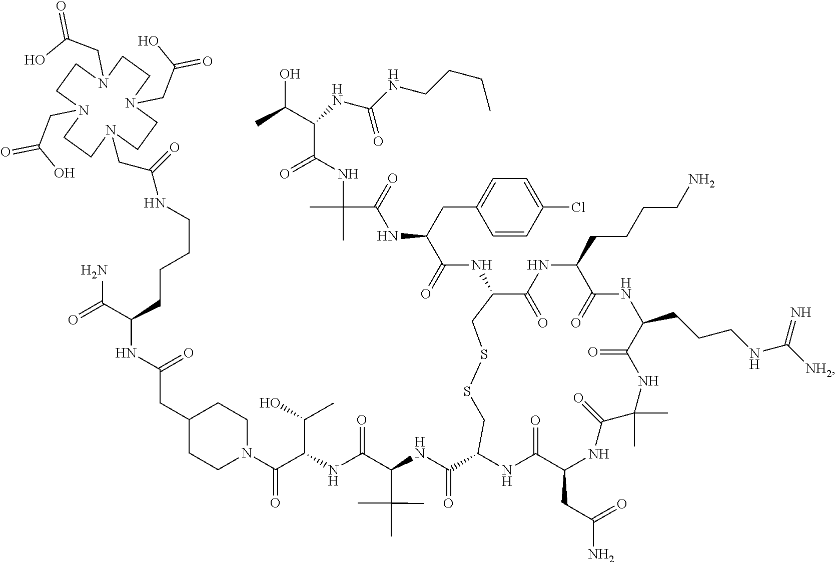 [SMILES: CCCCNC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Pmf-[Cys-Lys-Arg-Aib-Asn-Cys]-Tle-Thr-NH₂ (PSM-0372) of the following formula

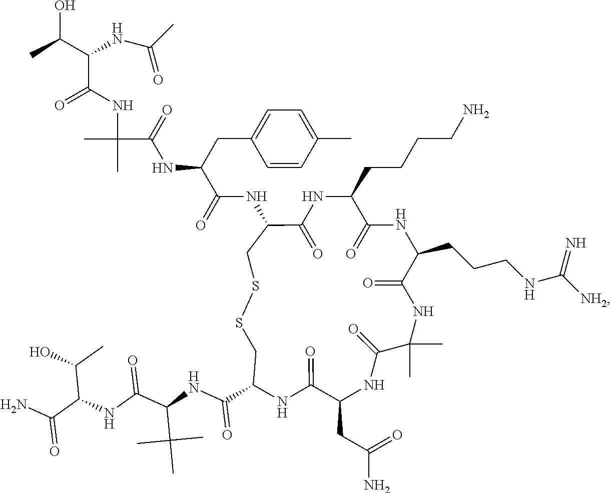 [SMILES: CC(=O)N[C@H](C(=O)NC(C)(C)C(=O)N[C@@H](Cc1ccc(C)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound LuDOTA-Cmp-Thr-Aib-Pcf-[Cys-Lys(Me)-Arg(Me)-Aib-Asn-Cys]-Tle-Thr-NH₂ (PSM-0373) of the following formula

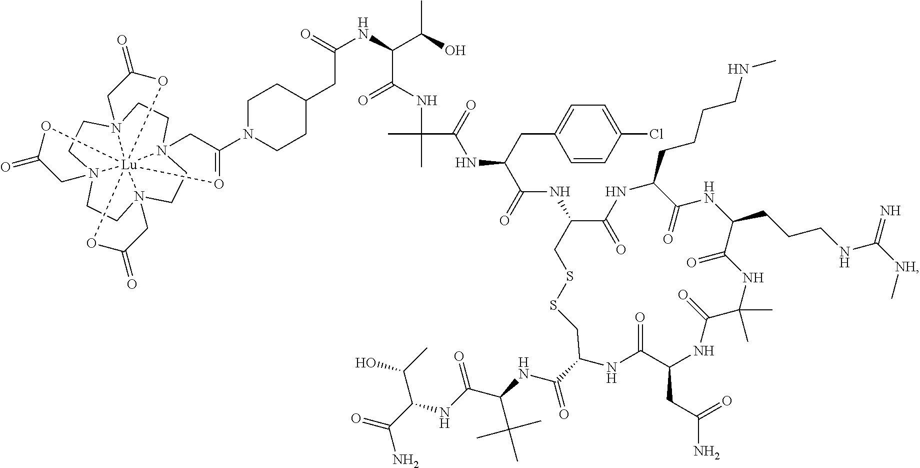

compound Ac-Thr-Aib-Phe-[Cys-Lys-Arg-Aib-Asn-Cys]-Tle-Thr-Asp-NH₂ (PSM-0374) of the following formula

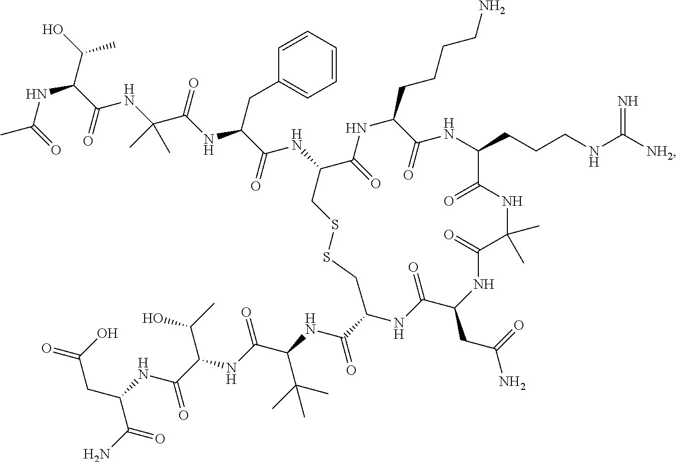 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Deg-Asn-Cys]-Tle-Thr-NH₂ (PSM-0375) of the following formula

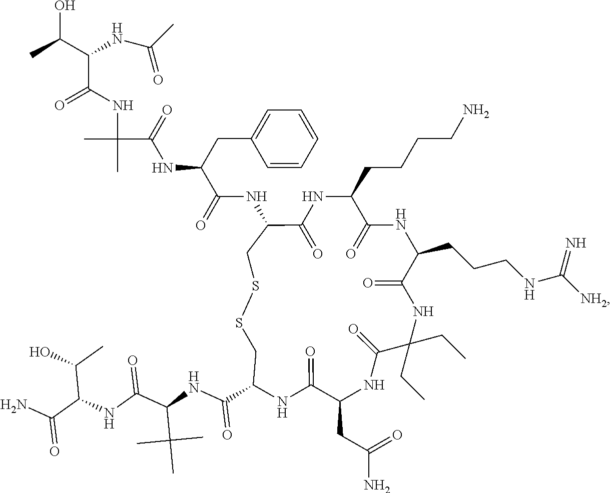 [SMILES: CCC1(CC)NC(=O)[C@H](CCCNC(=N)N)NC(=O)[C@H](CCCCN)NC(=O)[C@@H](NC(=O)[C@H](Cc2ccccc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC1=O]

compound Ac-Thr-Aib-Phe-[Cys-Lys-Arg-Aib-Asn-Cys]-Tle-Trp-NH₂ (PSM-0376) of the following formula

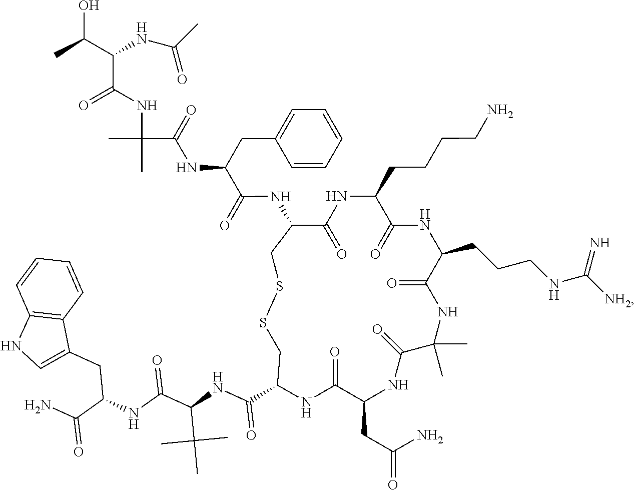 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@@H](Cc2c[nH]c3ccccc23)C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Aib-Asn-Cys]-Val-Nmt-NH₂ (PSM-0377) of the following formula

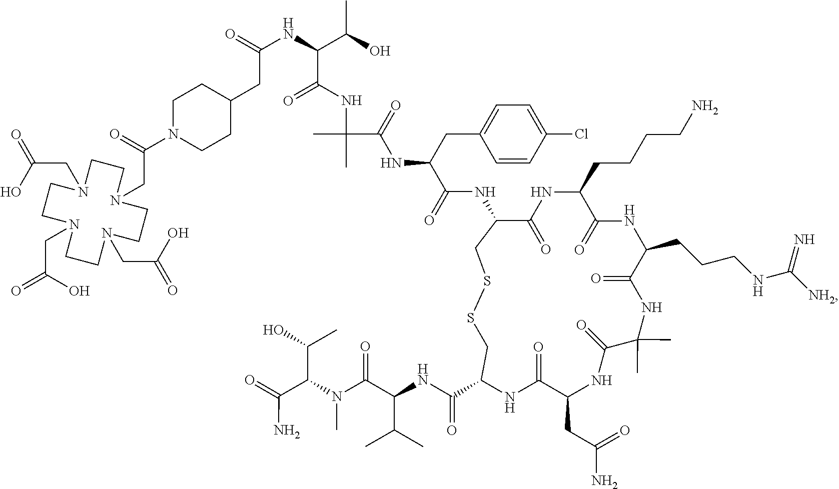 [SMILES: CC(C)[C@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(=O)N(C)[C@H](C(N)=O)[C@@H](C)O]

compound H-Cmp-Thr-Aib-Phe-[Cys-Lys-Arg-Aib-Asn-Cys]-Tle-Thr-Cmp-lys(DOTA)-NH₂ (PSM-0378) of the following formula

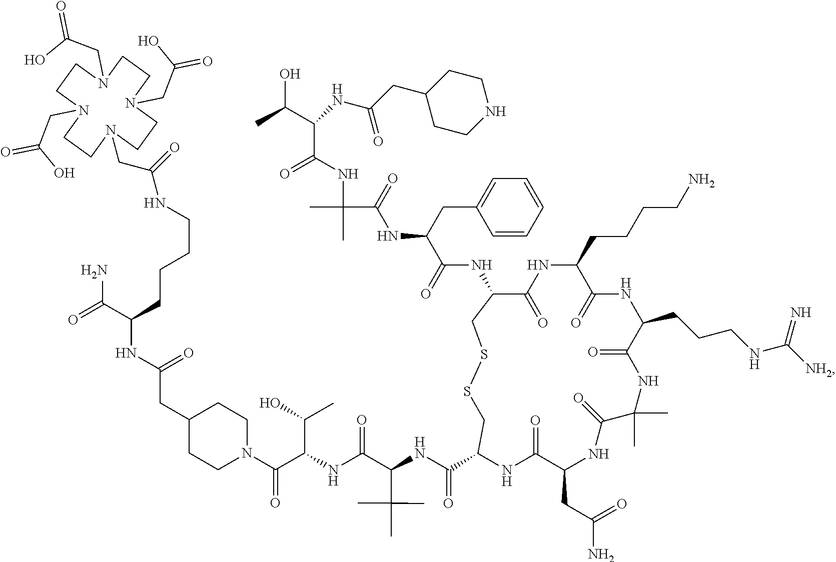 [SMILES: C[C@@H](O)[C@H](NC(=O)CC1CCNCC1)C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Cmp-Thr-Aib-Pcf-[Cys-Lys-Arg-Aib-Asn-Cys]-Tle-en-DOTA (PSM-0379) of the following formula

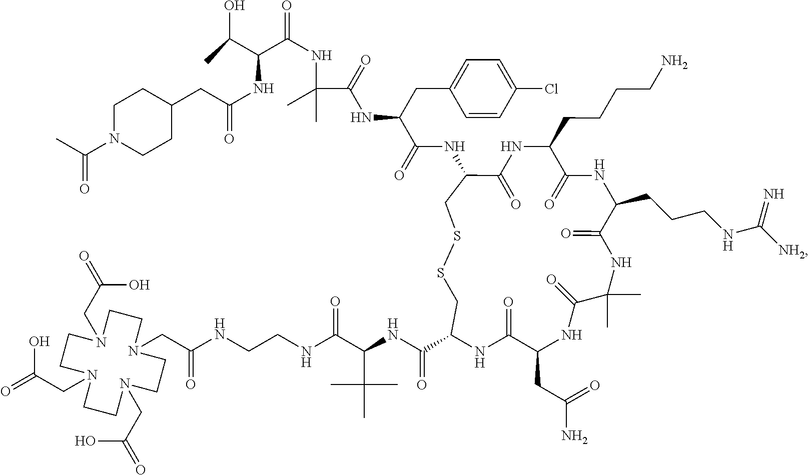 [SMILES: CC(=O)N1CCC(CC(=O)N[C@H](C(=O)NC(C)(C)C(=O)N[C@@H](Cc2ccc(Cl)cc2)C(=O)N[C@H]2CSSC[C@@H](C(=O)N[C@H](C(=O)NCCNC(=O)CN3CCN(CC(=O)O)CCN(CC(=O)O)CCN(CC(=O)O)CC3)C(C)(C)C)NC(=O)[C@H](CC(N)=O)NC(=O)C(C)(C)NC(=O)[C@H](CCCNC(=N)N)NC(=O)[C@H](CCCCN)NC2=O)[C@@H](C)O)CC1]

compound DOTA-Cmp-Thr-Aib-Pcf-[Cys-Lys(Me)-Arg(Me)-Aib-Asn-Cys]-Tle-NH₂ (PSM-0380) of the following formula

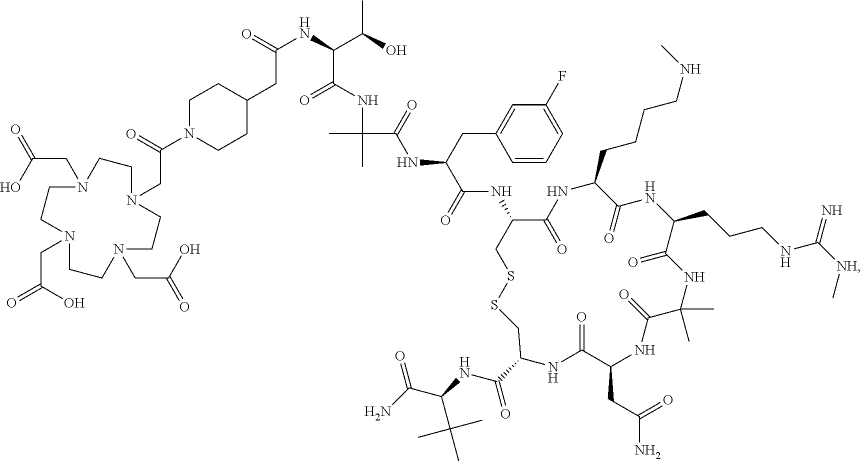 [SMILES: CNCCCC[C@@H]1NC(=O)[C@@H](NC(=O)[C@H](Cc2cccc(F)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound DOTA-Pamb-Aib-Pcf-[Cys-Lys-Arg-Aib-Asn-Cys]-Tle-NH₂ (PSM-0381) of the following formula

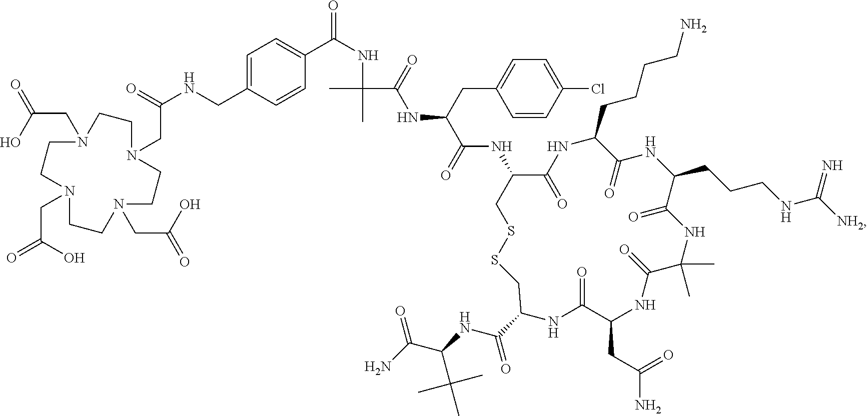 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Pcf-[Cys-Lys-Arg-Ams-Asn-Cys]-Tle-Thr-NH₂ (PSM-0382) of the following formula

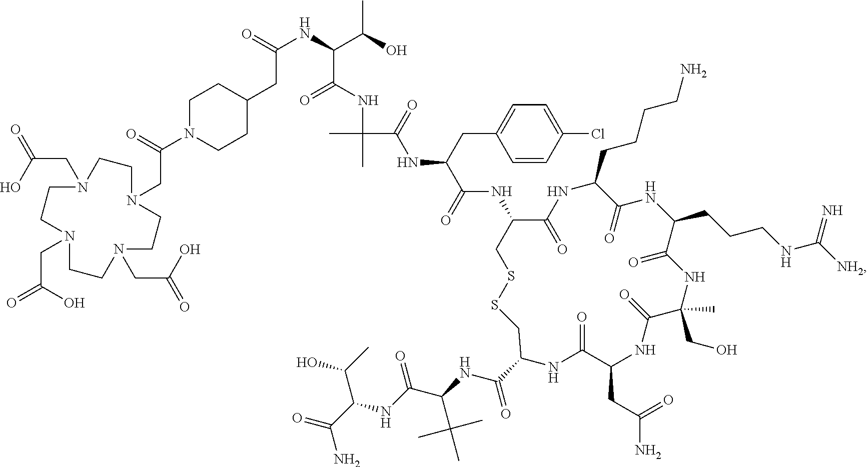 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@@](C)(CO)C(=O)N[C@@H](CC(N)=O)C(=O)N1)C(C)(C)C)C(N)=O]

compound Ac-Thr-Aib-Mcf-[Cys-Lys-Arg-Aib-Asn-Cys]-Tle-Thr-Asp-NH₂ (PSM-0383) of the following formula

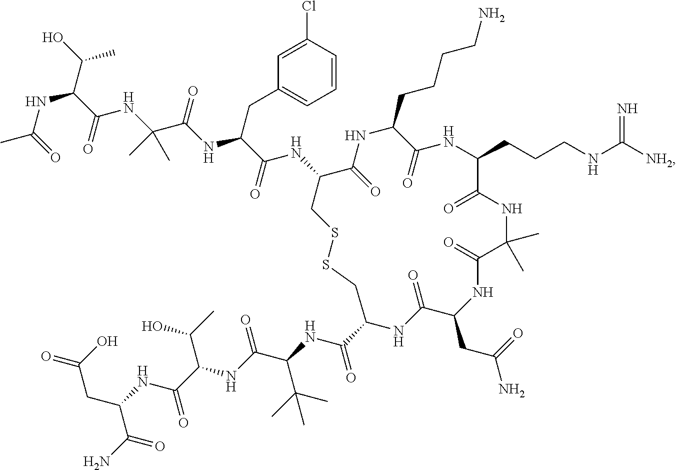 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Aib-Asn-Cys]-Tle-en (PSM-0384) of the following formula

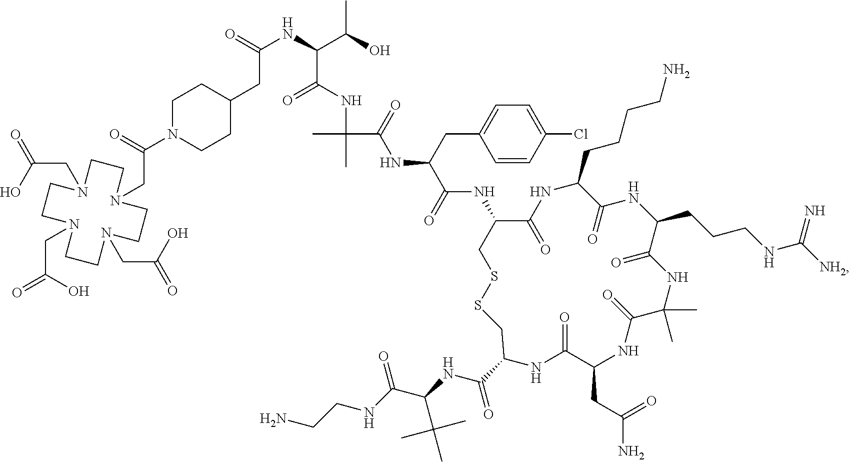 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccc(Cl)cc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Pho-[Cys-Lys-Gln-Lys(DOTA)-Asn-Cys]-Tle-Thr-NH₂ (PSM-0385) of the following formula

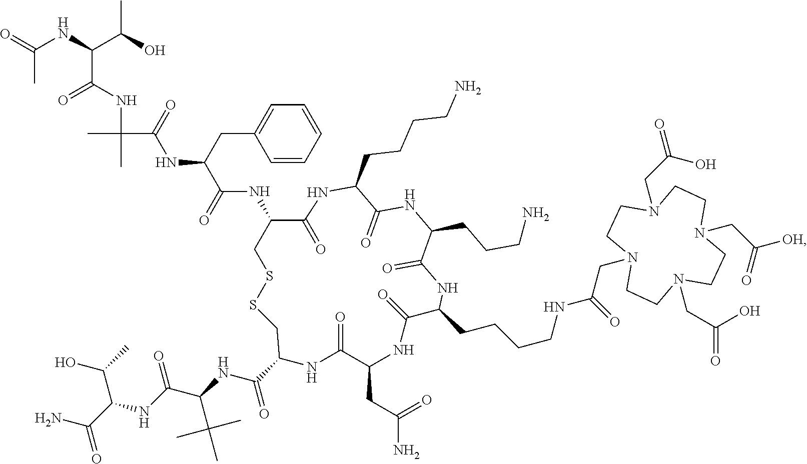 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CN2CCN(CC(=O)O)CCN(CC(=O)O)CCN(CC(=O)O)CC2)NC(=O)[C@H](CCCN)NC(=O)[C@H](CCCCN)NC1=O)[C@@H](C)O]

compound DOTA-Cmp-Thr-Aib-Fso-[Cys-Lys-Arg-Aib-Asn-Cys]-Tle-Thr-NH₂ (PSM-0386) of the following formula

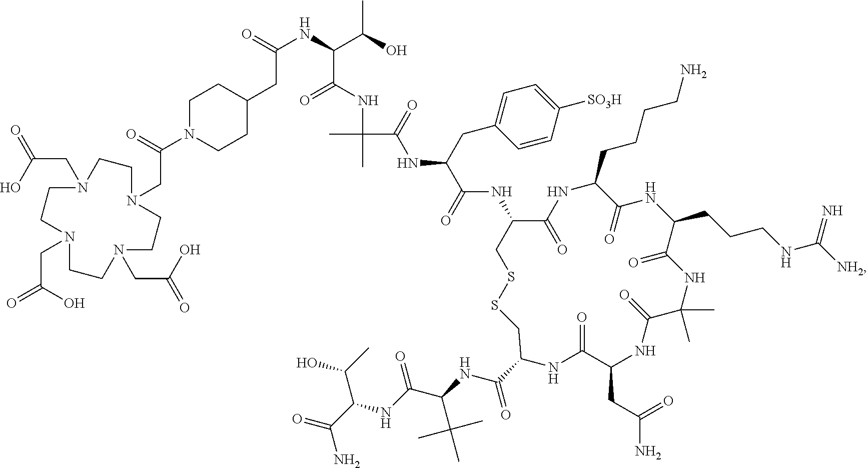 [SMILES: C[C@@H](O)[C@H](NC(=O)[C@@H](NC(=O)[C@@H]1CSSC[C@H](NC(=O)[C@H](Cc2ccc(S(=O)(=O)O)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Miy-[Cys-Lys(Me)-Arg(Me)-Aib-Asn-Cys]-Tle-Thr-NH₂ (PSM-0387) of the following formula

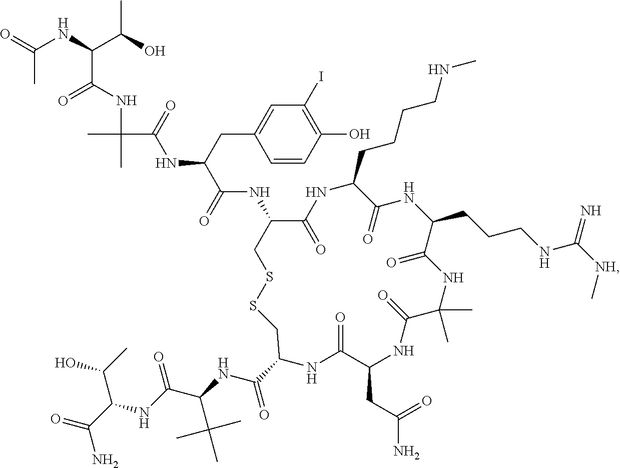 [SMILES: CNCCCC[C@@H]1NC(=O)[C@@H](NC(=O)[C@H](Cc2ccc(O)c(I)c2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound Ac-Thr-Aib-Phe-[Cys-Lys-Arg-Aib-Asn-Cys]-Tle-Pro-NH₂ (PSM-0388) of the following formula

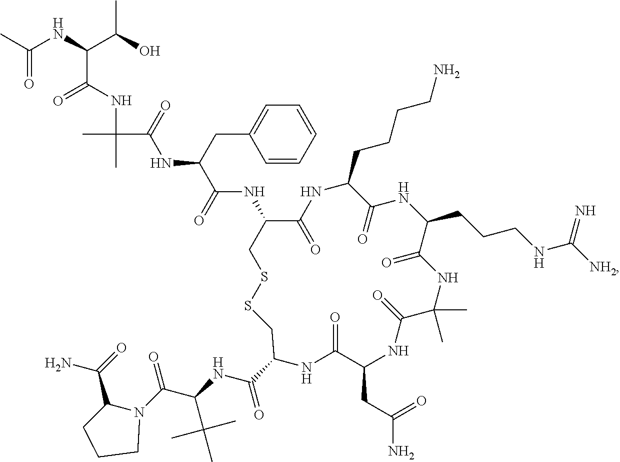

compound Ac-Thr-Ala-Phe-[Cys-Lys-Arg-Ala-Asn-Cys]-Nle-Thr-asp-NH₂ (PSM-0389) of the following formula

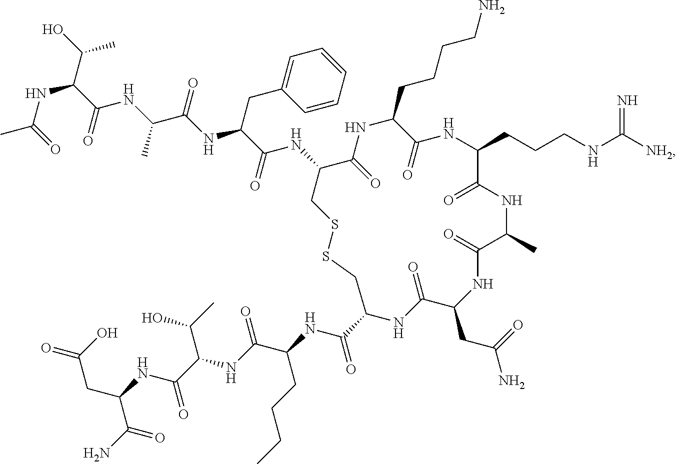 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@H](CC(=O)O)C(N)=O)[C@@H](C)O]

compound DOTA-Cmp-Tle-Aib-6Clw-[Cys-Lys(Me)-Arg(Me)-Aib-Asn-Cys]-Tle-NH₂ (PSM-0390) of the following formula

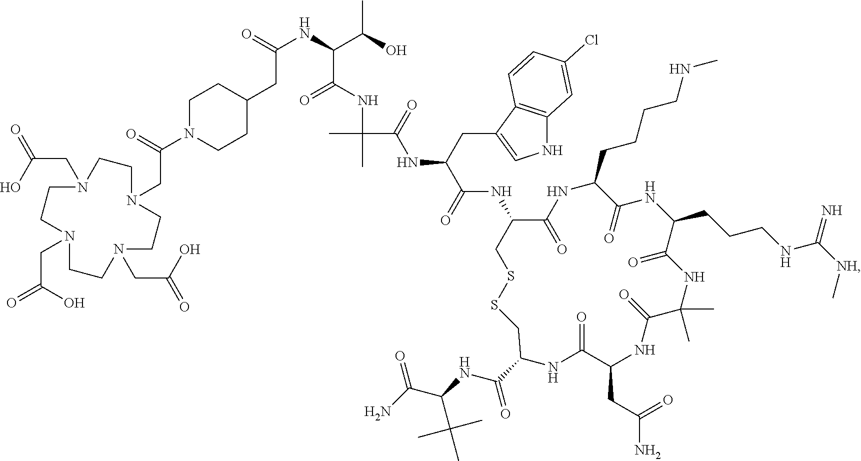 [SMILES: CNCCCC[C@@H]1NC(=O)[C@@H](NC(=O)[C@H](Cc2c[nH]c3cc(Cl)ccc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Aib-Phe-[Cys-Lys-Arg-Aib-Asn-Cys]-Tle-Thr-Ape-DOTA (PSM-0391) of the following formula

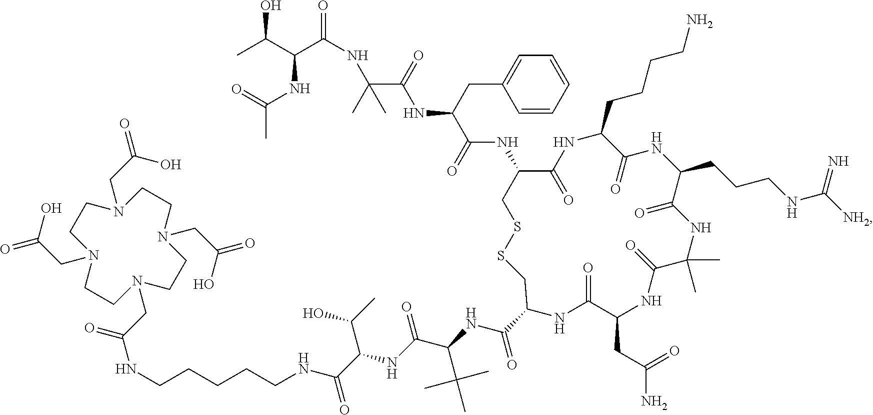 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCCCNC(=O)CN2CCN(CC(=O)O)CCN(CC(=O)O)CCN(CC(=O)O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-4Tfp-Asn-Cys]-Tle-Thr-Asp-NH₂ (PSM-0392) of the following formula

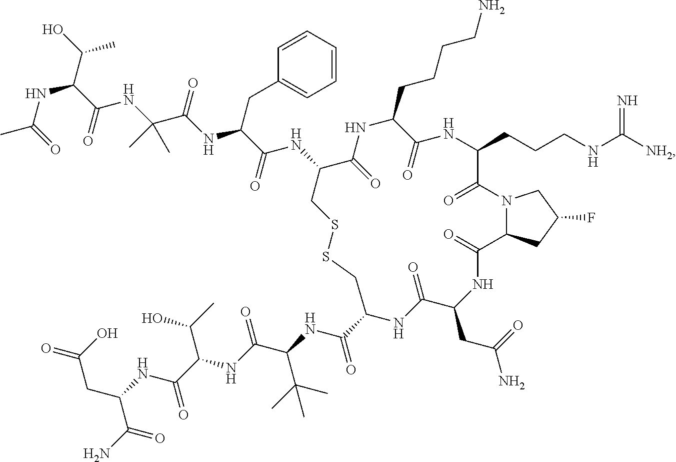 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@@H]2C[C@@H](F)CN2C(=O)[C@H](CCCNC(=N)N)NC(=O)[C@H](CCCCN)NC1=O)[C@@H](C)O]

compound Ac-Thr-Aib-Pcf-[Cys-Lys-Arg-Aib-Asn-Cys]-Tle-en (PSM-0393) of the following formula

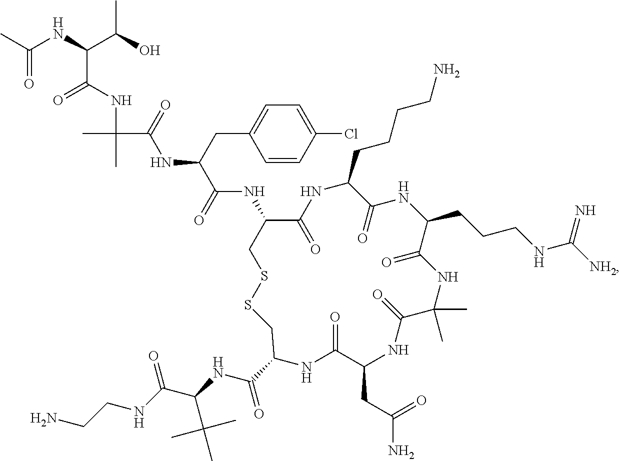 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Aib-Thr-NH₂ (PSM-0394) of the following formula

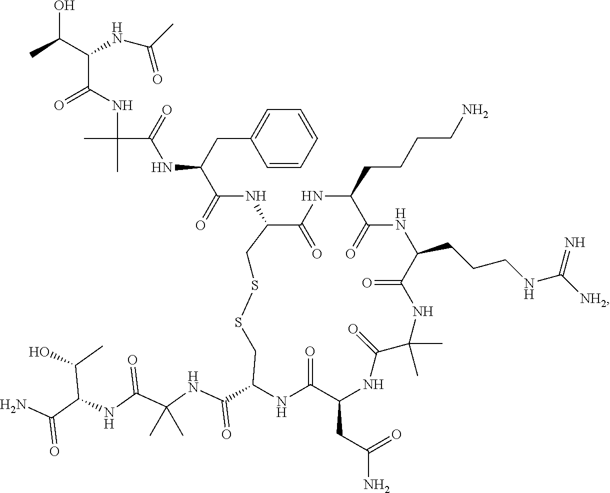 [SMILES: CC(=O)N[C@H](C(=O)NC(C)(C)C(=O)N[C@@H](Cc1ccccc1)C(=O)N[C@H]1CSSC[C@@H](C(=O)NC(C)(C)C(=O)N[C@H](C(N)=O)[C@@H](C)O)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pff-[Cys-Lys(Me)-Arg(Me)-Aib-Asn-Cys]-Tle-NH₂ (PSM-0395) of the following formula

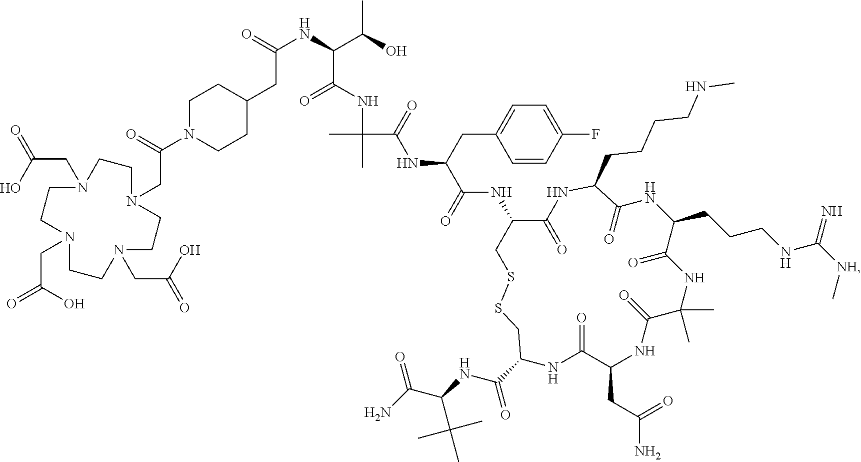 [SMILES: CNCCCC[C@@H]1NC(=O)[C@@H](NC(=O)[C@H](Cc2ccc(F)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound DOTA-Cmp-Thr-Aib-Mtf-[Cys-Lys(Me)-Arg(Me)-Aib-Asn-Cys]-Tle-NH₂ (PSM-0396) of the following formula

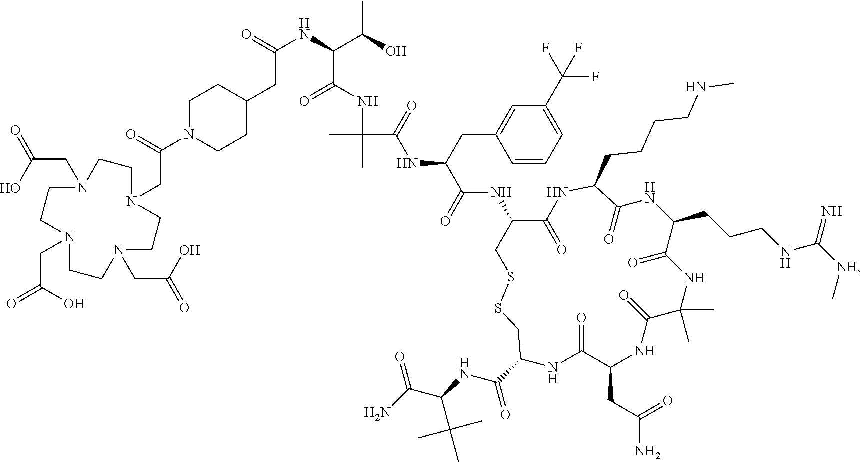 [SMILES: CNCCCC[C@@H]1NC(=O)[C@@H](NC(=O)[C@H](Cc2cccc(C(F)(F)F)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Aib-Phe-[Cys-Lys-Arg-Lys(DOTA)-Asn-Cys]-Tle-Thr-Asp-NH₂ (PSM-0397) of the following formula

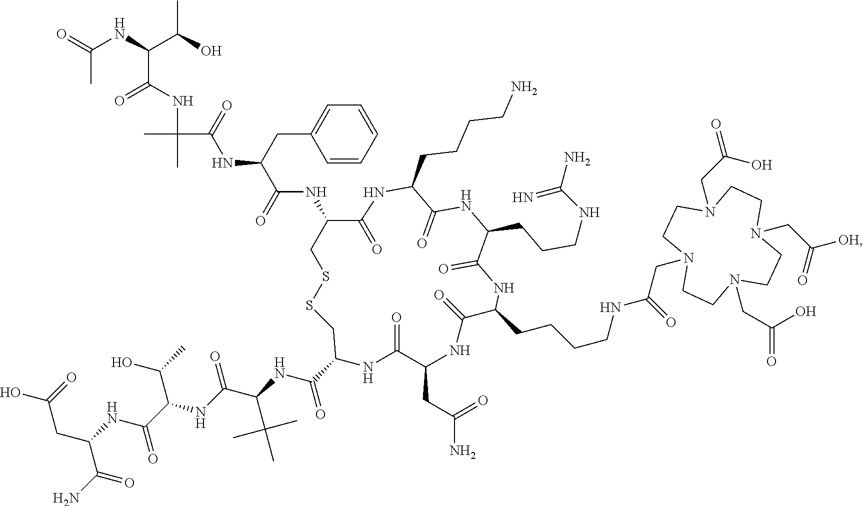 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](CCCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound DOTA-Ttds-Thr-Aib-Phe-[Cys-Lys-Arg-Aib-Asn-Cys]-Tle-Thr-Asp-NH₂ (PSM-0398) of the following formula

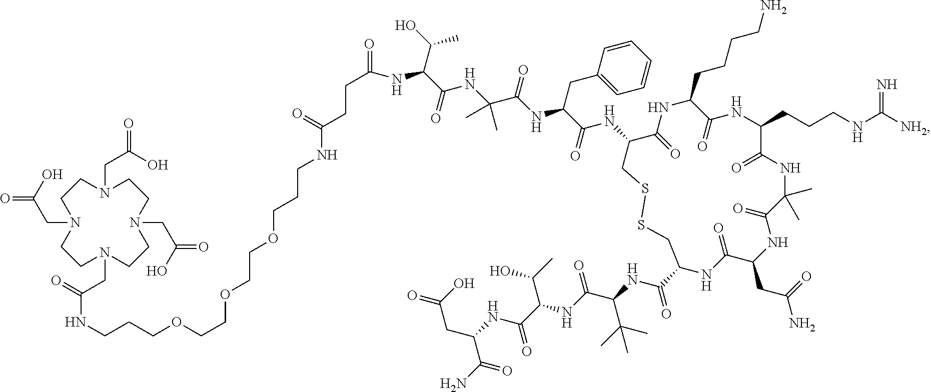 [SMILES: C[C@@H](O)[C@H](NC(=O)CCC(=O)NCCCOCCOCCOCCCNC(=O)CN1CCN(CC(=O)O)CCN(CC(=O)O)CCN(CC(=O)O)CC1)C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound InDOTA-Cmp-Thr-Aib-Pcf-[Cys-Lys-Cit-Aib-Asn-Cys]-Tle-Thr-NH₂ (PSM-0399) of the following formula

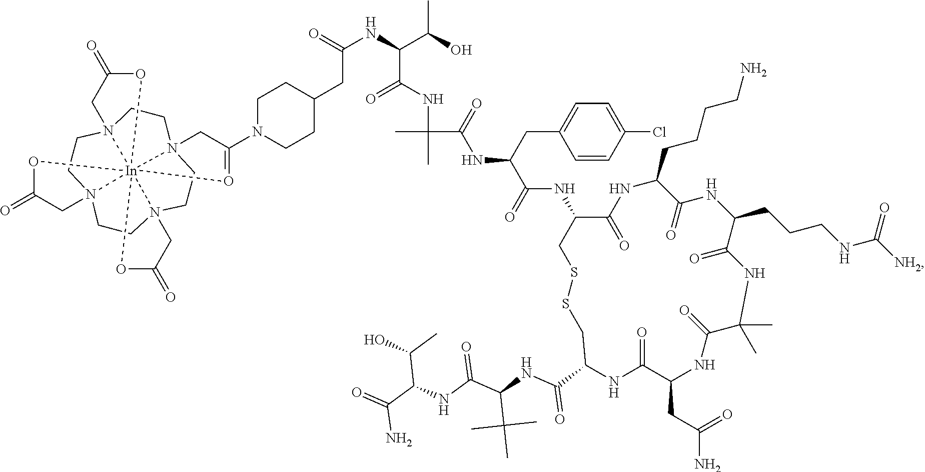

compound Hex-Thr-Aib-Pcf-[Cys-Lys-Arg-Aib-Asn-Cys]-Tle-Thr-lys (DOTA-Cmp)-NH₂ (PSM-0400) of the following formula

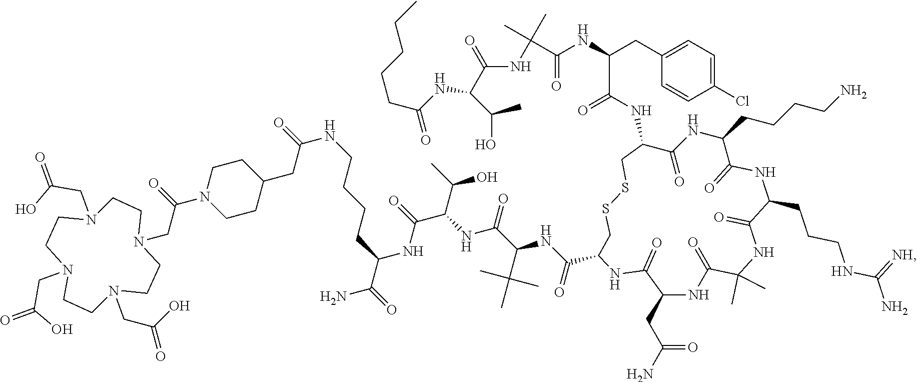 [SMILES: CCCCCC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys(DOTA-020c)-Asn-Cys]-Tle-Thr-NH₂ (PSM-0401) of the following formula

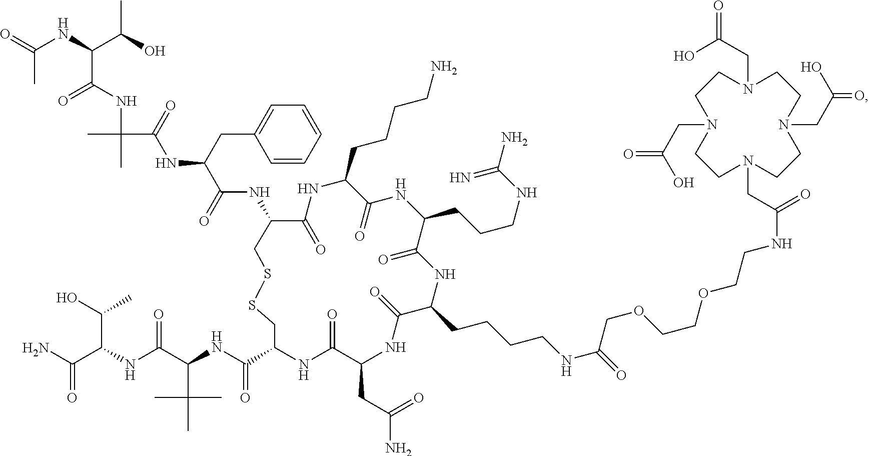 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)COCCOCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-asn-Cys]-Tle-Thr-NH₂ (PSM-0402) of the following formula

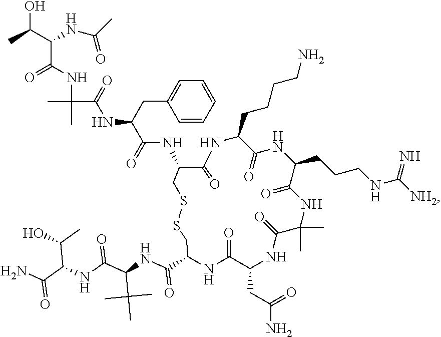 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Oic-Asn-Cys]-Tle-Thr-Asp-NH₂ (PSM-0403) of the following formula

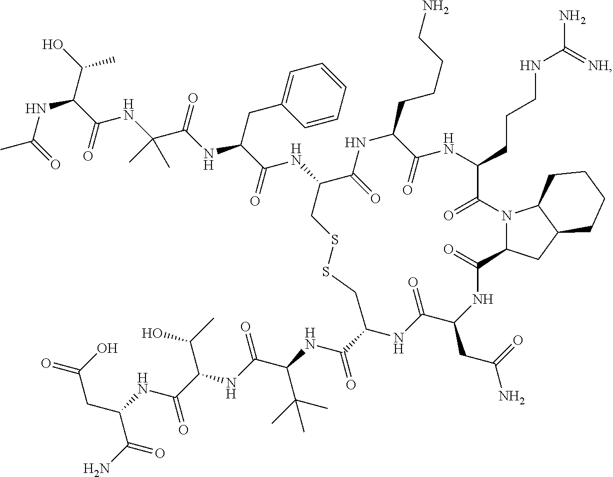 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@@H]2C[C@@H]3CCCC[C@@H]3N2C(=O)[C@H](CCCNC(=N)N)NC(=O)[C@H](CCCCN)NC1=O)[C@@H](C)O]

compound Ac-Aib-Pcf-[Cys-Lys-Arg-Aib-Asn-Cys]-Tle-Thr-lys(DOTA)-NH₂ (PSM-0404) of the following formula

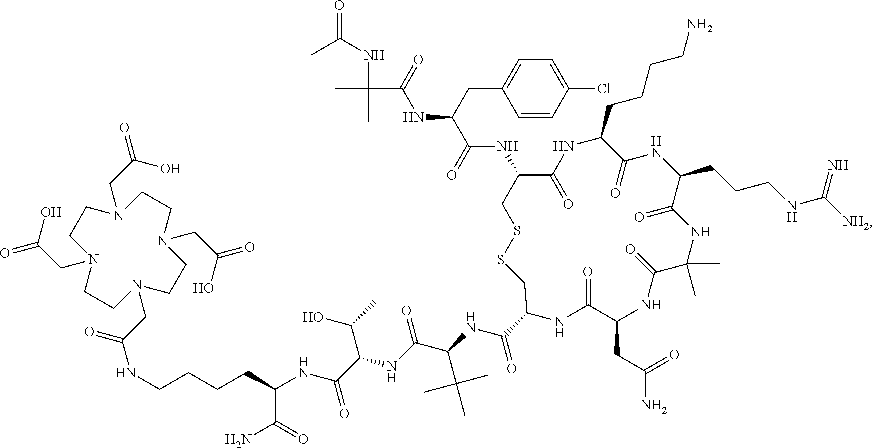 [SMILES: CC(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Hex-Thr-Ala-Phe-[Cys-Lys-Arg-Ala-Asn-Cys]-Leu-Thr-Asp-Ttds-lys(DOTA)-NH₂ (PSM-0405) of the following formula

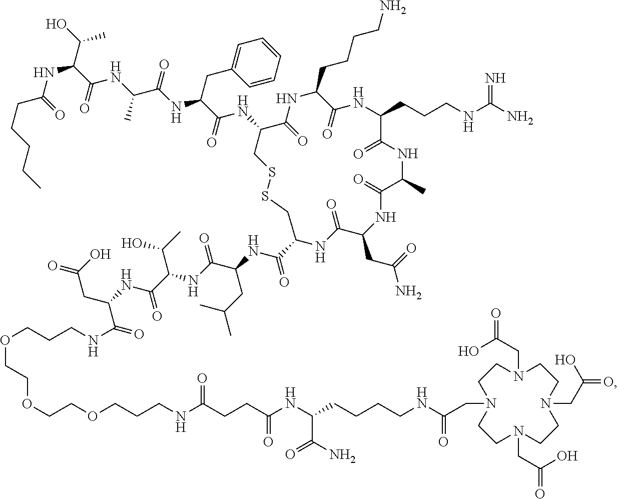 [SMILES: CCCCCC(=O)N[C@H](C(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-5Clw-[Cys-Lys-Cit-Aib-Asn-Cys]-Tle-Thr-NH₂ (PSM-0406) of the following formula

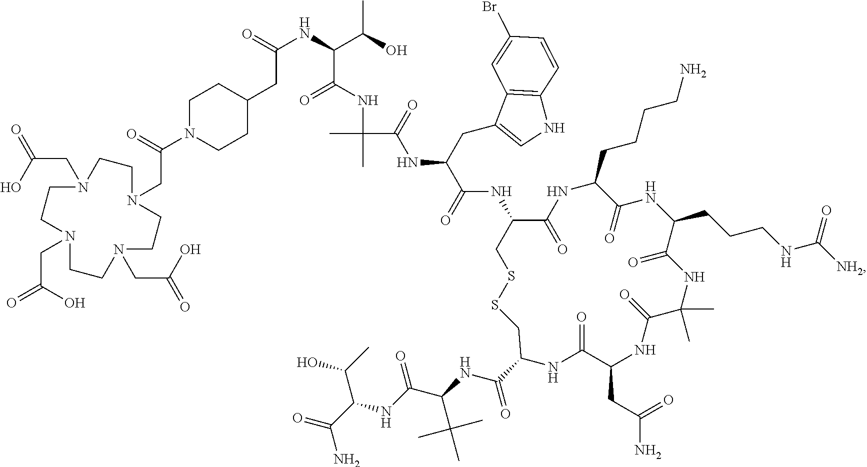 [SMILES: C[C@@H](O)[C@H](NC(=O)[C@@H](NC(=O)[C@@H]1CSSC[C@H](NC(=O)[C@H](Cc2c[nH]c3ccc(Br)cc23)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Cmp-Thr-Ams-Pcf-[Cys-Lys-Arg-Ams-Asn-Cys]-Tle-Thr-NH₂ (PSM-0407) of the following formula

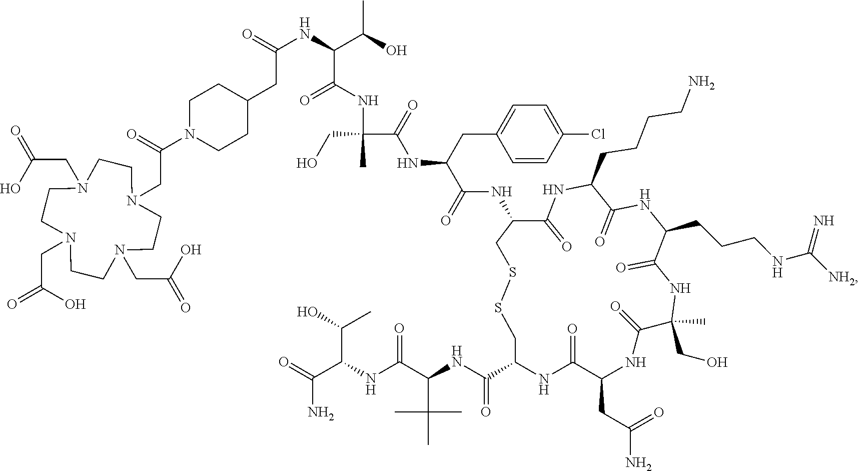 [SMILES: C[C@@H](O)[C@H](NC(=O)[C@@H](NC(=O)[C@@H]1CSSC[C@H](NC(=O)[C@H](Cc2ccc(Cl)cc2)NC(=O)[C@](C)(CO)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@@](C)(CO)C(=O)N[C@@H](CC(N)=O)C(=O)N1)C(C)(C)C)C(N)=O]

compound Ac-Thr-Nmg-Phe-[Cys-Lys-Arg-Aib-Asn-Cys]-Tle-Thr-NH₂ (PSM-0408) of the following formula

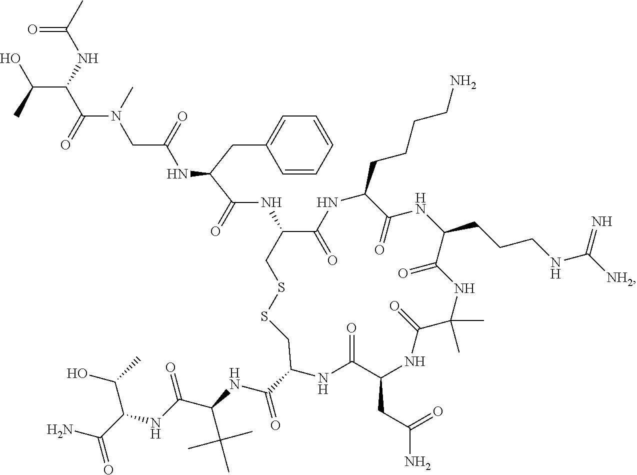 [SMILES: CC(=O)N[C@H](C(=O)N(C)CC(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Opa-[Cys-Lys(Me)-Arg(Me)-Aib-Asn-Cys]-Tle-NH₂ (PSM-0409) of the following formula

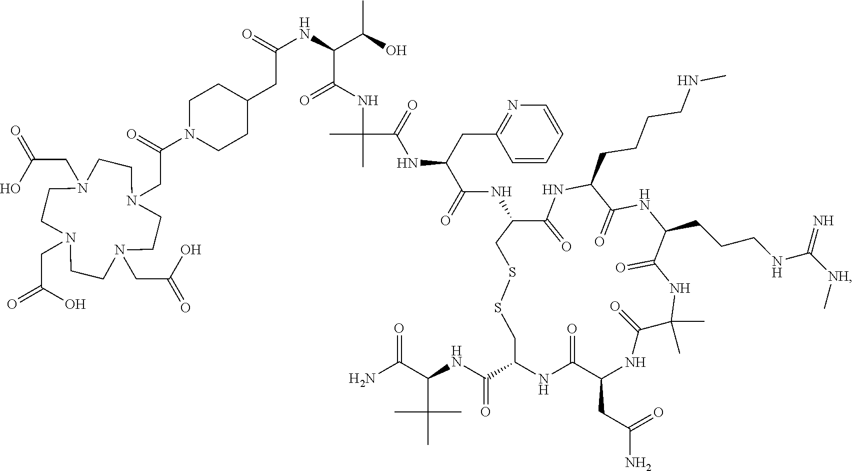 [SMILES: CNCCCC[C@@H]1NC(=O)[C@@H](NC(=O)[C@H](Cc2ccccn2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound DOTA-Cmp-Thr-Aib-Pcf-[Cys-Lys(Me)-Arg-Aib-Asn-Cys]-Tle-Thr-NH₂ (PSM-0410) of the following formula

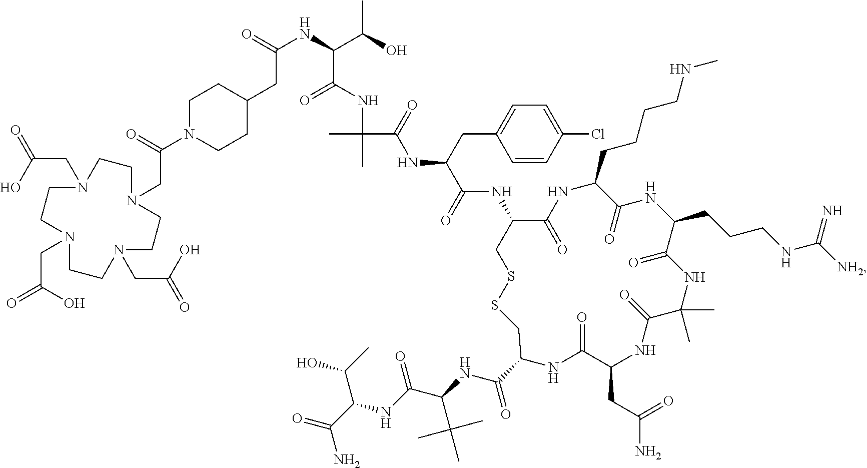 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC1=O]

compound Ac-Thr-Aib-Hfe-[Cys-Lys-Arg-Aib-Asn-Cys]-Tle-Thr-NH₂ (PSM-0411) of the following formula

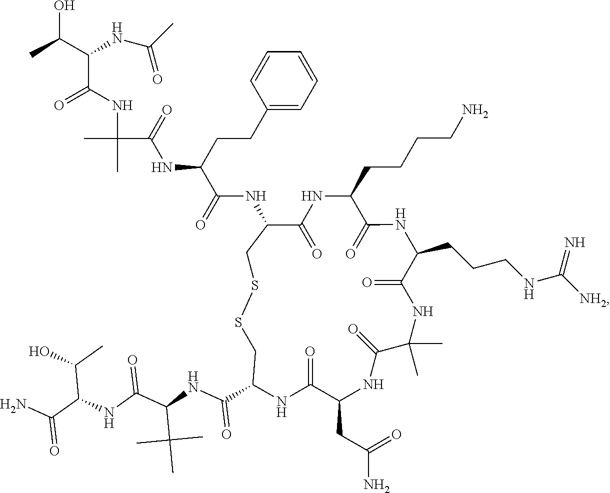 [SMILES: CC(=O)N[C@H](C(=O)NC(C)(C)C(=O)N[C@@H](CCc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Mmf-[Cys-Lys-Arg-Aib-Asn-Cys]-Tle-Thr-NH₂ (PSM-0412) of the following formula

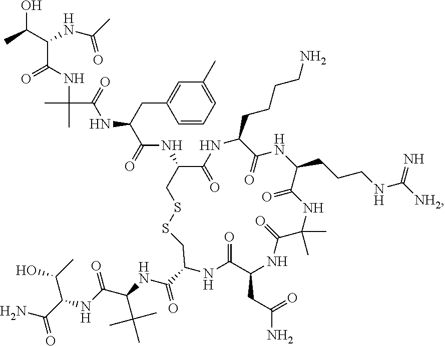 [SMILES: CC(=O)N[C@H](C(=O)NC(C)(C)C(=O)N[C@@H](Cc1cccc(C)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-PPAc-Aib-Phe-[Cys-Lys-Arg-Aib-Asn-Cys]-Tle-Thr-NH₂ (PSM-0413) of the following formula

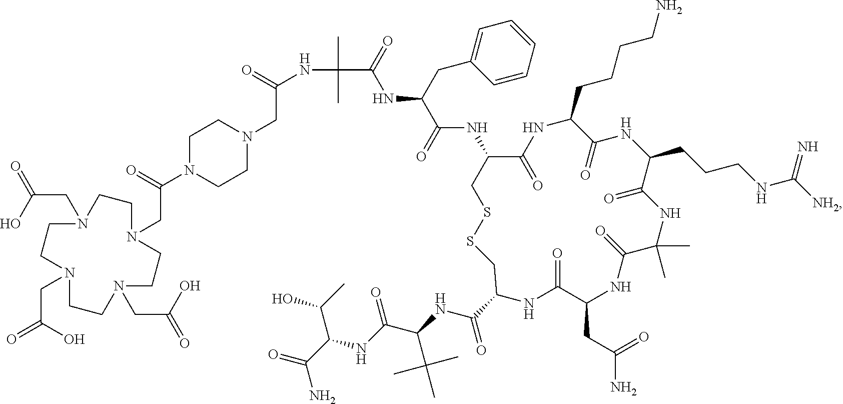 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CN2CCN(C(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Ala-Amf-[Cys-Lys-Arg-Aib-Asn-Cys]-Tle-Thr-NH₂ (PSM-0414) of the following formula

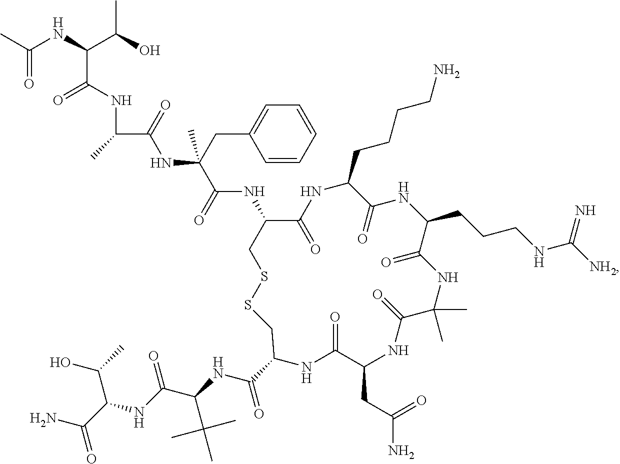 [SMILES: CC(=O)N[C@H](C(=O)N[C@@H](C)C(=O)N[C@@](C)(Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys(DOTA-Cmp)-Asn-Cys]-Tle-Thr-NH₂ (PSM-0415) of the following formula

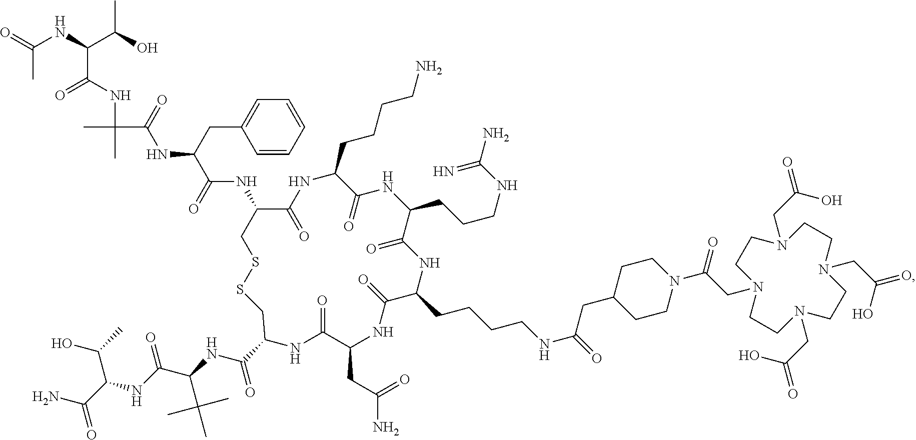 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CC2CCN(C(=O)CN3CCN(CC(=O)O)CCN(CC(=O)O)CCN(CC(=O)O)CC3)CC2)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Glu-Asn-Cys]-Tle-Thr-NH₂ (PSM-0416) of the following formula

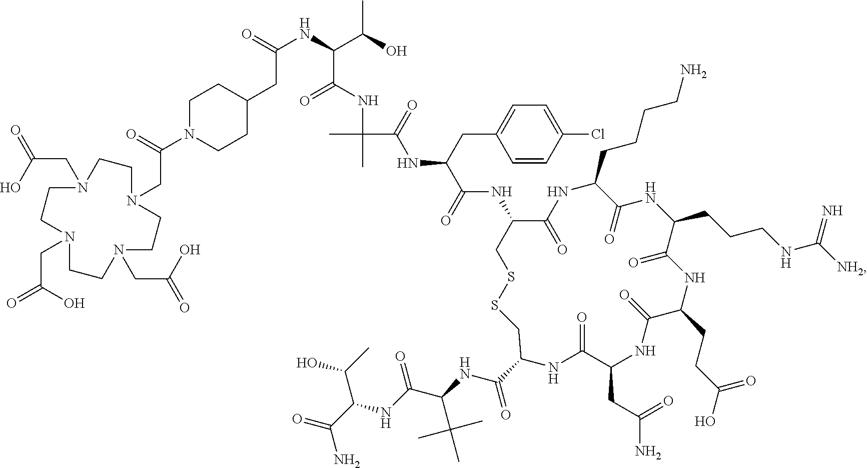 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)N[C@@H](CCC(=O)O)C(=O)N[C@@H](CC(N)=O)C(=O)N1)C(C)(C)C)C(N)=O]

compound LuDOTA-Ttds-Thr-Aib-Phe-[Cys-Lys-Arg-Ala-Asn-Cys]-Tle-Thr-NH₂ (PSM-0418) of the following formula

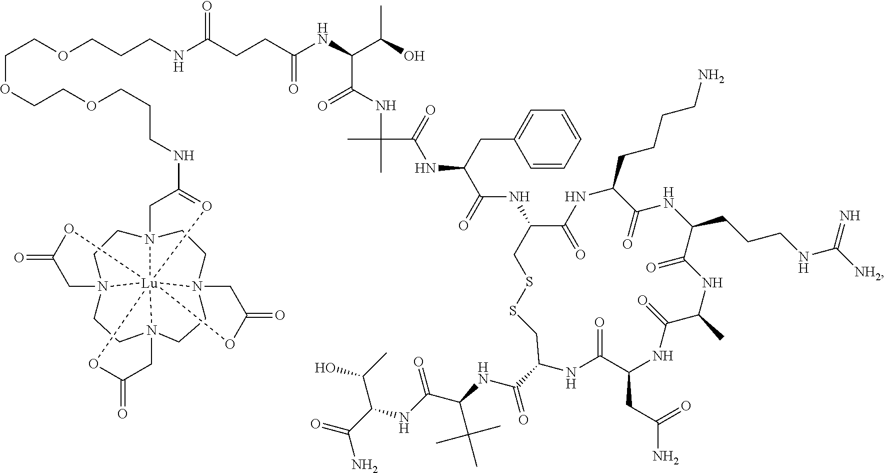

compound Ac-Thr-Aib-Phe-[Cys-Lys-Arg-Lys(DOTA-Pamb)-Asn-Cys]-Tle-Thr-NH₂ (PSM-0419) of the following formula

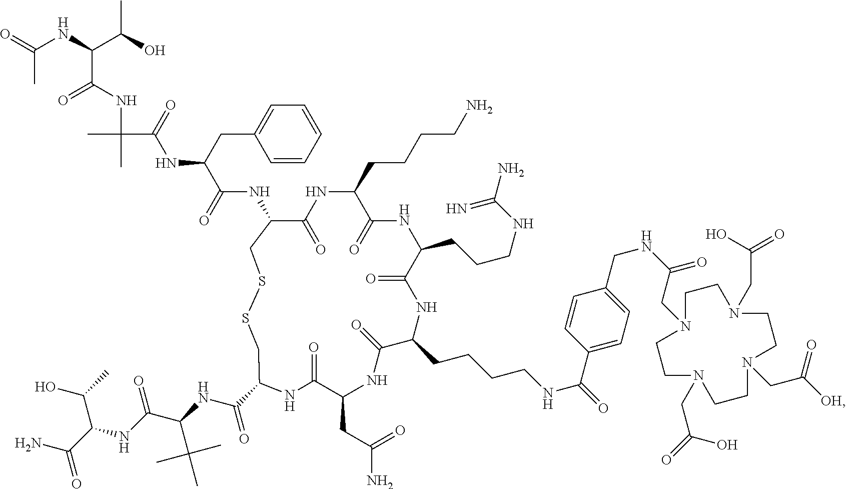 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)c2ccc(CNC(=O)CN3CCN(CC(=O)O)CCN(CC(=O)O)CCN(CC(=O)O)CC3)cc2)NC(=O)[C@H](CCCNC(=N)N)NC(=O)[C@H](CCCCN)NC1=O)[C@@H](C)O]

compound DOTA-Cmp-Thr-Aib-Pcf-[Smc-Lys(Me)-Arg(Me)-Aib-Asn-Cys]-Tle-NH₂ (alternative: DOTA-Cmp-Thr-Aib-Pcf-[Cys-Lys(Me)-Arg(Me)-Aib-Asn-Smc]-Tle-NH₂) (PSM-0420) of the following formula

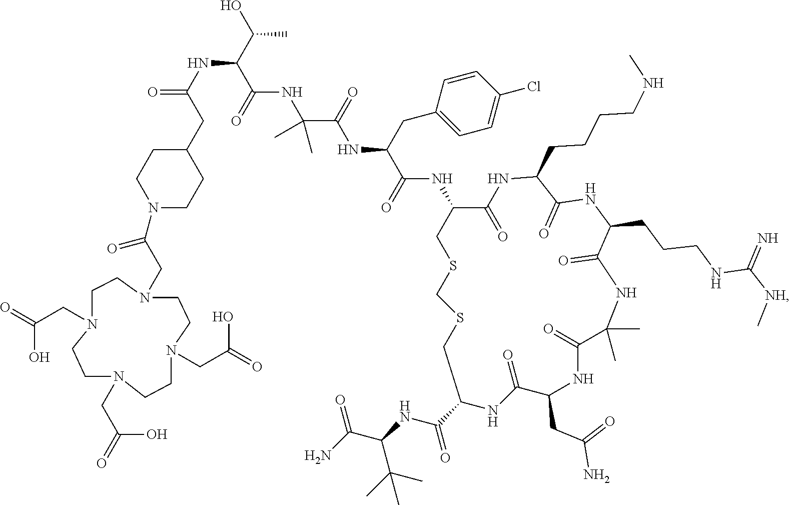 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSCSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Aib-Pcf-[Cys-Lys-Cit-Aib-Asn-Cys]-leu-Thr-NH₂ (PSM-0421) of the following formula

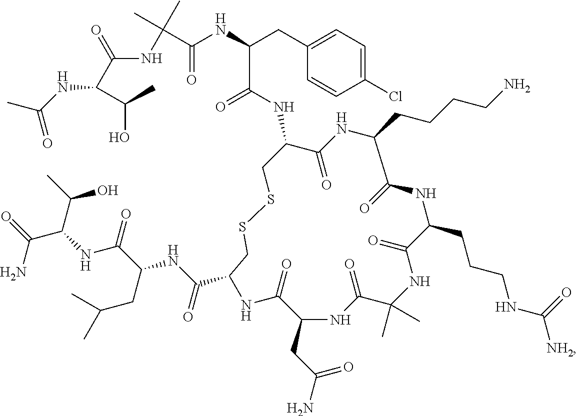

compound Ac-Thr-Aib-Phe-[Cys-Tap-Arg-Ala-Asn-Cys]-Tle-Thr-Asp-NH₂ (PSM-0422) of the following formula

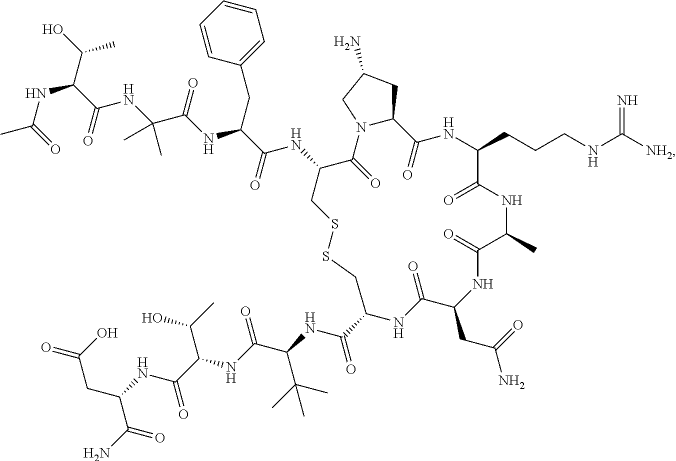 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@@H]2C[C@@H](N)CN2C1=O)[C@@H](C)O]

compound Ac-Thr-Aib-Phe-[Cys-Lys-Gln-Lys(DOTA-Ttds)-Asn-Cys]-Tle-Thr-NH₂ (PSM-0423) of the following formula

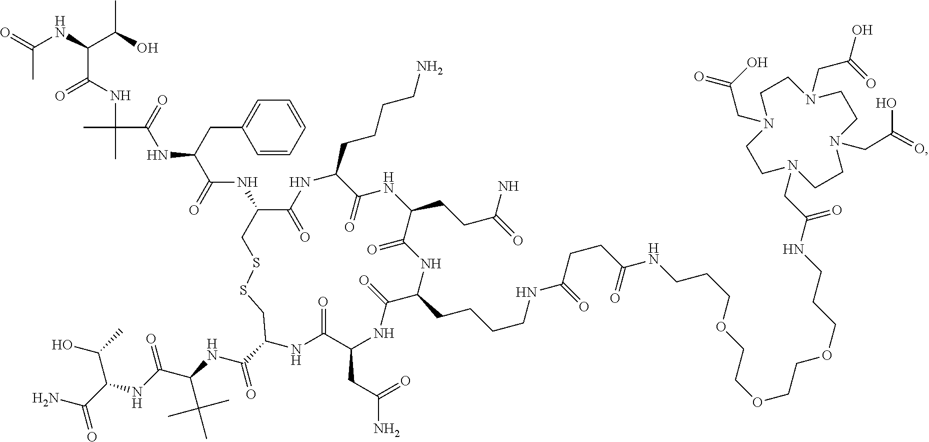 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CCC(=O)NCCCOCCOCCOCCCNC(=O)CN2CCN(CC(=O)O)CCN(CC(=O)O)CCN(CC(=O)O)CC2)NC(=O)[C@H](CCC(N)=O)NC(=O)[C@H](CCCCN)NC1=O)[C@@H](C)O]

compound Ac-Aib-Pcf-[Cys-Lys-Arg-Aib-Asn-Cys]-Tle-NH₂ (PSM-0424) of the following formula

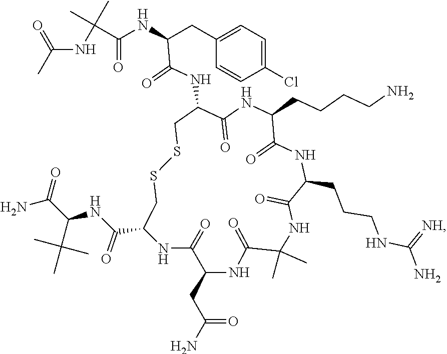 [SMILES: CC(=O)NC(C)(C)C(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Hex-Thr-Aib-Phe-[Cys-Lys-Arg-Aib-Asn-Cys]-Tle-Thr-Asp-Ttds-lys(DOTA)-NH₂ (PSM-0425) of the following formula

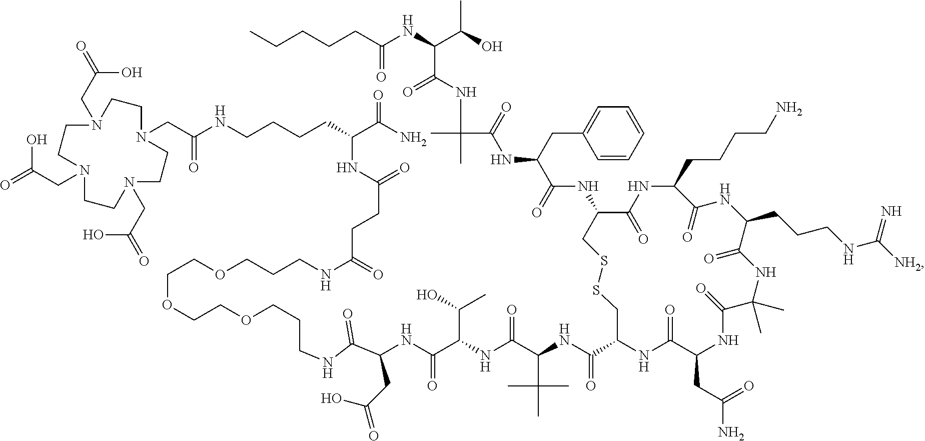 [SMILES: CCCCCC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Bal-NH₂ (PSM-0426) of the following formula

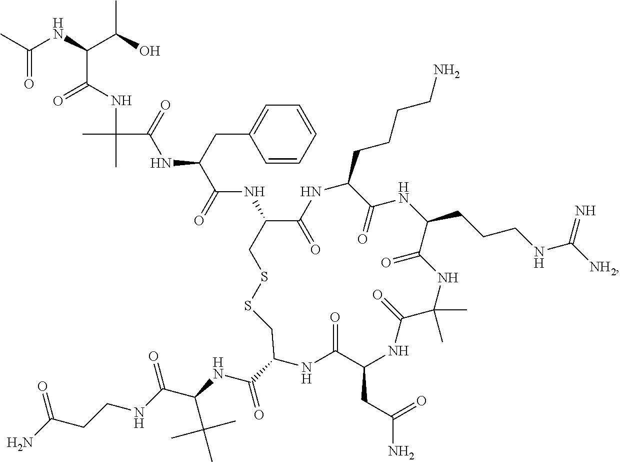 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound nBuCAyl-Thr-Aib-Pcf-[Cys-Lys-Opy-Aib-Asn-Cys]-Tle-Thr-Cmp-lys(DOTA)-NH₂ (PSM-0427) of the following formula

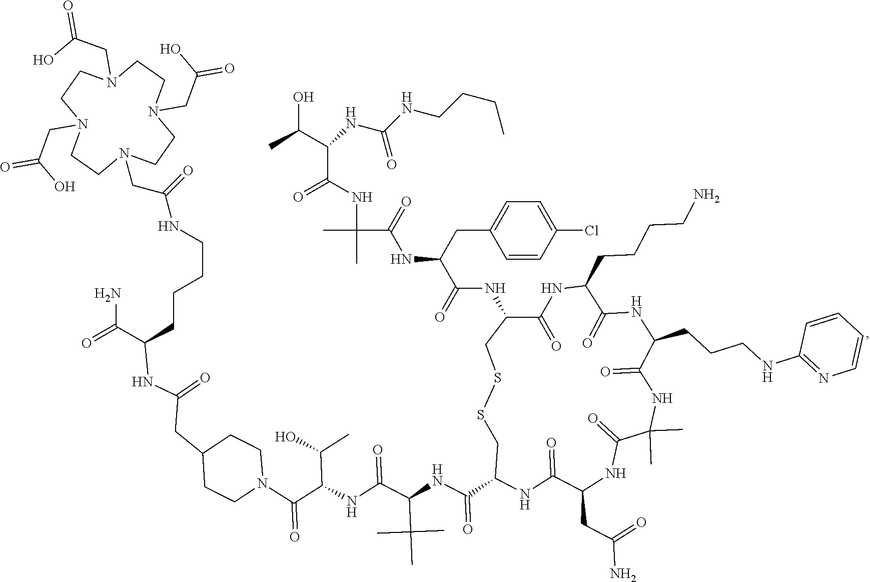 [SMILES: CCCCNC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNc2ccccn2)NC(=O)[C@H](CCCCN)NC1=O)[C@@H](C)O]

compound Ac-Thr-Aib-Phe-[Cys-Lys-Arg-Aib-Asn-Cys]-Tle-Thr-Cmp-lys(DOTA)-NH₂ (PSM-0428) of the following formula

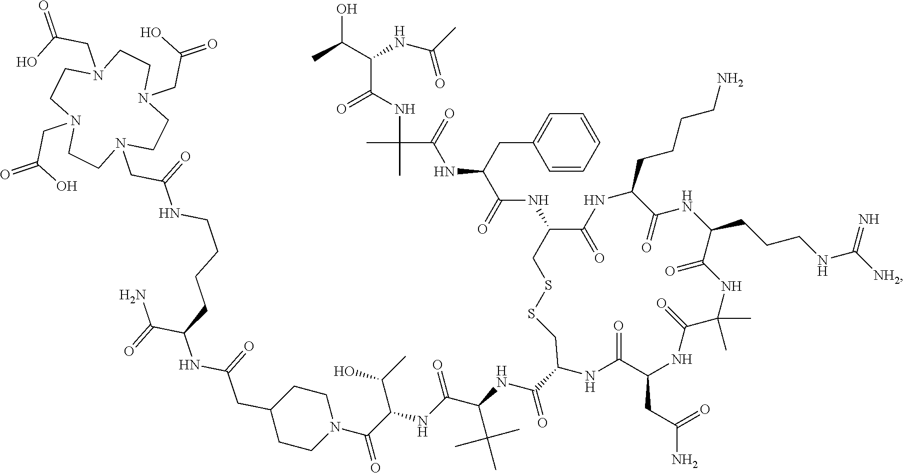 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound AF488Ahx-Ttds-Ttds-Thr-Aib-Pcf-[Cys-Lys(Me)-Arg(Me)-Aib-Asn-Cys]-Tle-Thr-NH₂ (PSM-0430) of the following formula

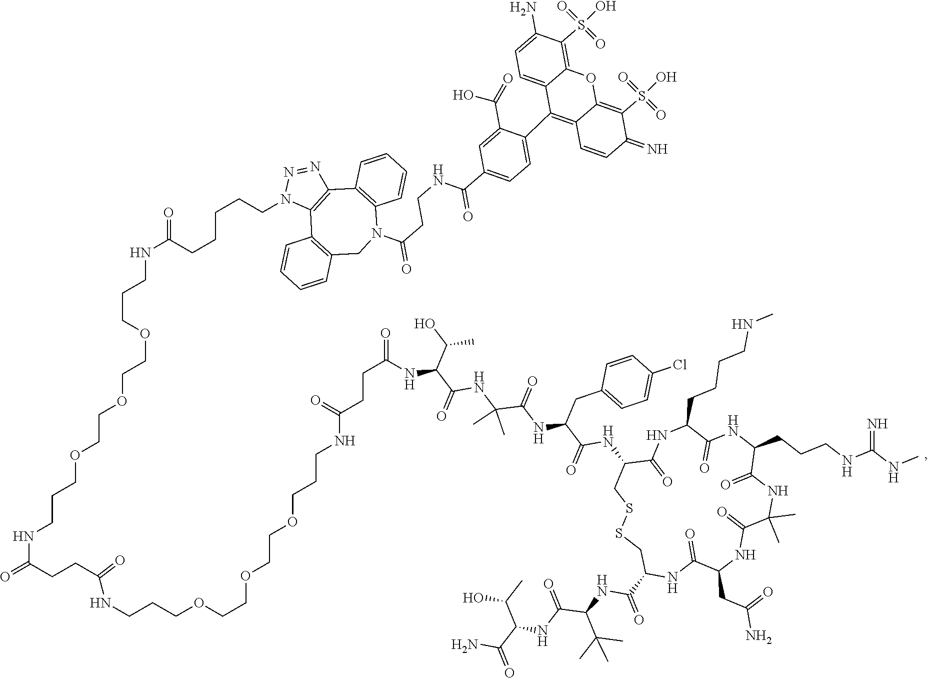 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CCC(=O)NCCCOCCOCCOCCCNC(=O)CCC(=O)NCCCOCCOCCOCCCNC(=O)CCCCCn2nnc3c2-c2ccccc2CN(C(=O)CCNC(=O)c2ccc(-c4c5ccc(=N)c(S(=O)(=O)O)c-5oc5c(S(=O)(=O)O)c(N)ccc45)c(C(=O)O)c2)c2ccccc2-3)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound DOTA-Cmp-Thr-Aib-Mcf-[Cys-Lys-Cit-Aib-Asn-Cys]-Tle-Thr-NH₂ (PSM-0431) of the following formula

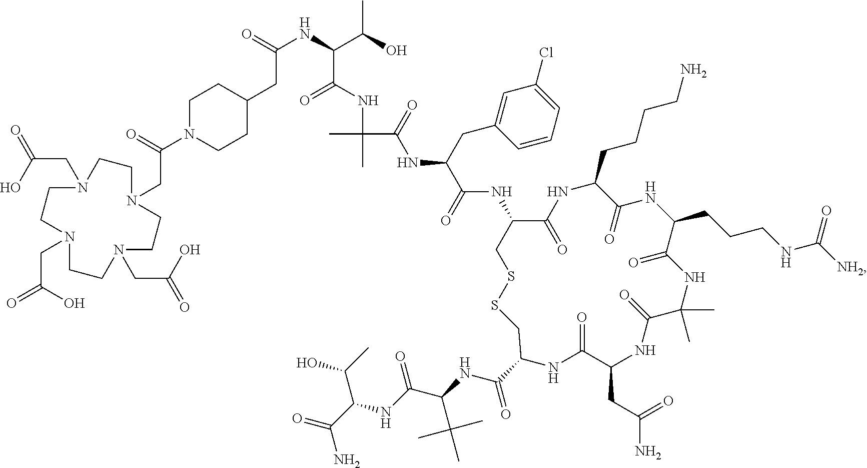 [SMILES: C[C@@H](O)[C@H](NC(=O)[C@@H](NC(=O)[C@@H]1CSSC[C@H](NC(=O)[C@H](Cc2cccc(Cl)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Pamb-Aib-Pcf-[Cys-Lys-Arg-Aib-Asn-Cys]-Tle-Bal-NH₂ (PSM-0432) of the following formula

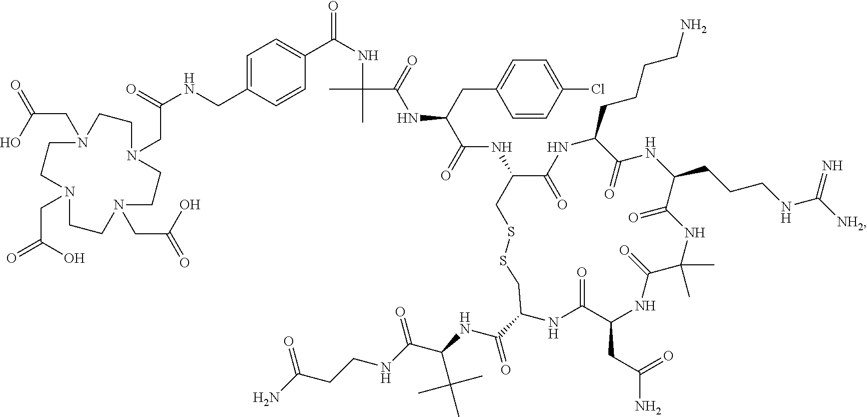 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccc(Cl)cc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Pcf-[Cys-Lys-Arg-Aib-Asn-Cys]-Tle-NH₂ (PSM-0433) of the following formula

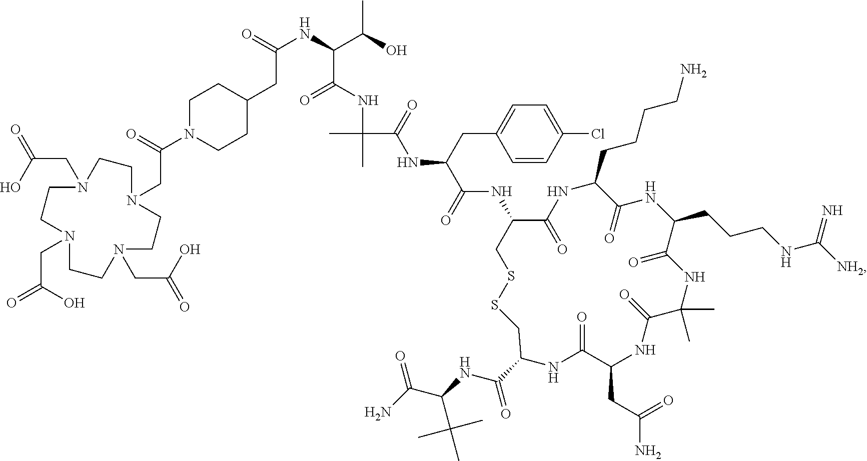 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound nBuCAyl-Thr-Aib-Phe-[Cys-Lys-Cit-Aib-Asn-Cys]-Tle-Thr-Cmp-lys(DOTA)-NH₂ (PSM-0434) of the following formula

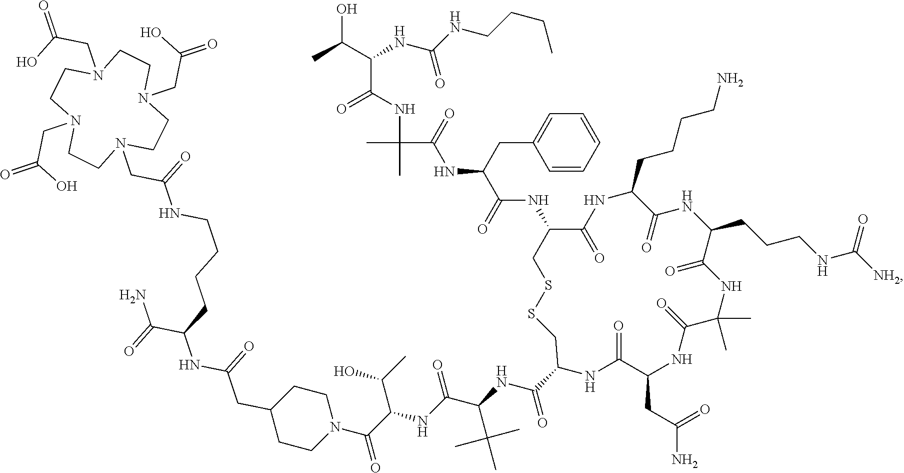 [SMILES: CCCCNC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(N)=O)NC(=O)[C@H](CCCCN)NC1=O)[C@@H](C)O]

compound Ac-Thr-Aib-Phe-[Cys-Gln-Arg-Ala-Asn-Cys]-Tle-Thr-Asp-NH₂ (PSM-0435) of the following formula

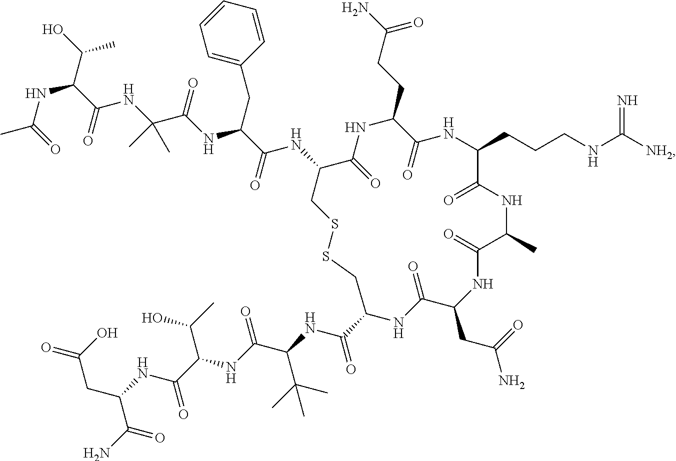 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCC(N)=O)NC1=O)[C@@H](C)O]

compound Ac-Thr-Ala-phe-[Cys-Lys-Arg-Ala-Asn-Cys]-Nle-Thr-Asp-NH₂ (PSM-0436) of the following formula

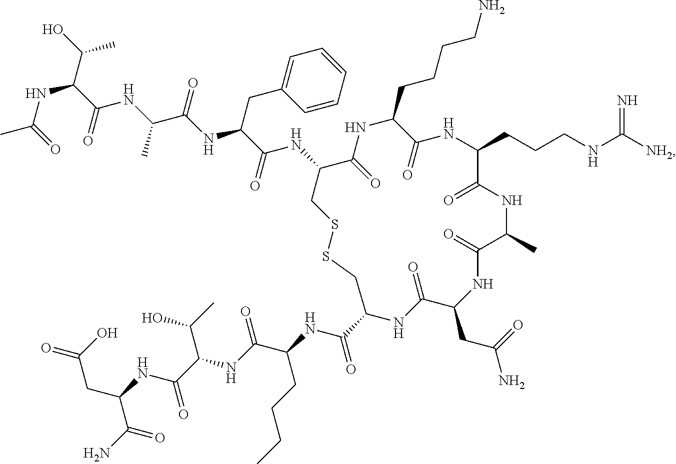 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@H](CC(=O)O)C(N)=O)[C@@H](C)O]

compound DOTA-Bal-Aib-Phe-[Cys-Lys-Arg-Aib-Asn-Cys]-Tle-Thr-NH₂ (PSM-0437) of the following formula

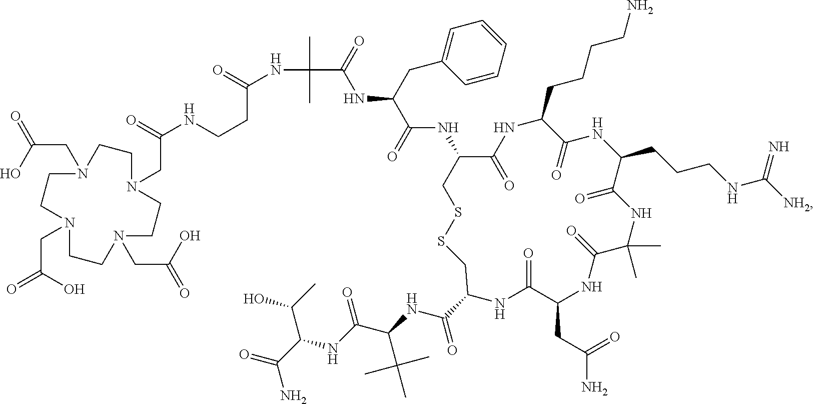 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CCNC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Cys-Lys-Arg-Lys (LuDOTA-020c)-Asn-Cys]-Tle-Thr-NH₂ (PSM-0438) of the following formula

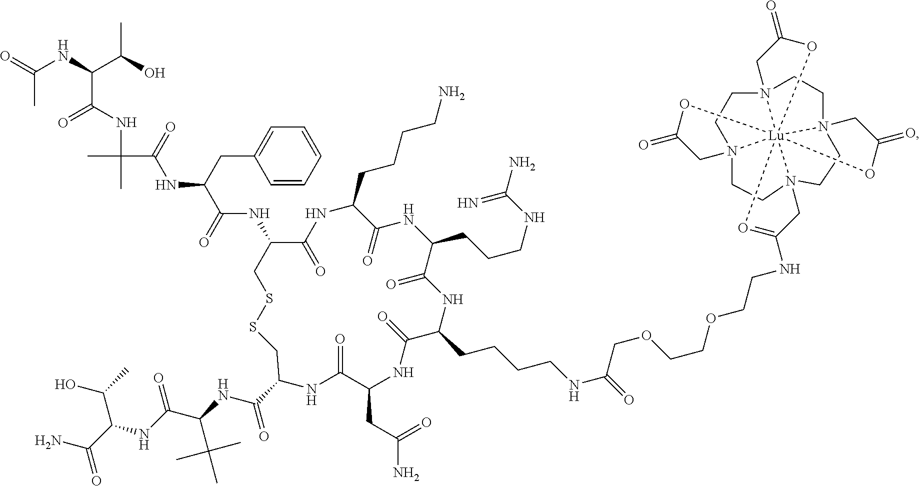

compound Ac-Thr-Aib-Phe-[Cys-Aph-Arg-Ala-Asn-Cys]-Tle-Thr-Asp-NH₂ (PSM-0439) of the following formula

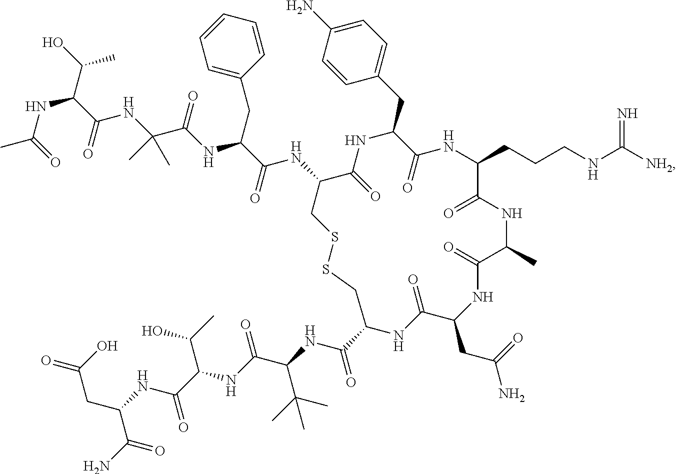 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](Cc2ccc(N)cc2)NC1=O)[C@@H](C)O]

compound Ac-Thr-Aib-Phe-[Cys-Lys-Arg-Aib-Asn-Cys]-Tle-Thr-Cmp-lys (InDOTA)-NH₂ (PSM-0440) of the following formula

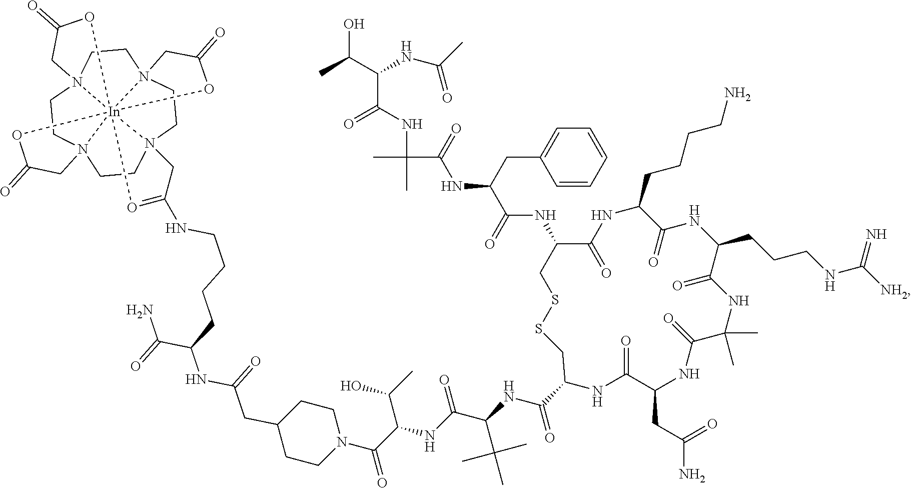

compound Ac-Thr-Aib-Cys(Bzl)-[Cys-Lys-Arg-Aib-Asn-Cys]-Tle-Thr-NH₂ (PSM-0441) of the following formula

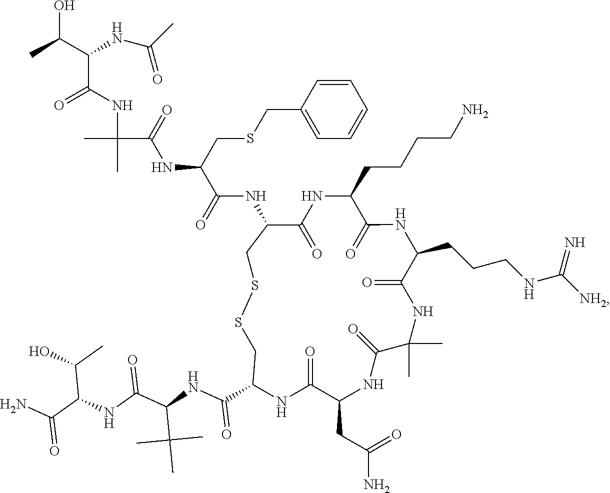 [SMILES: CC(=O)N[C@H](C(=O)NC(C)(C)C(=O)N[C@@H](CSCc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Phe-[Cys-Lys-Arg-Aib-Asn-Cys]-Tle-Thr-Asp-NH₂ (PSM-0442) of the following formula

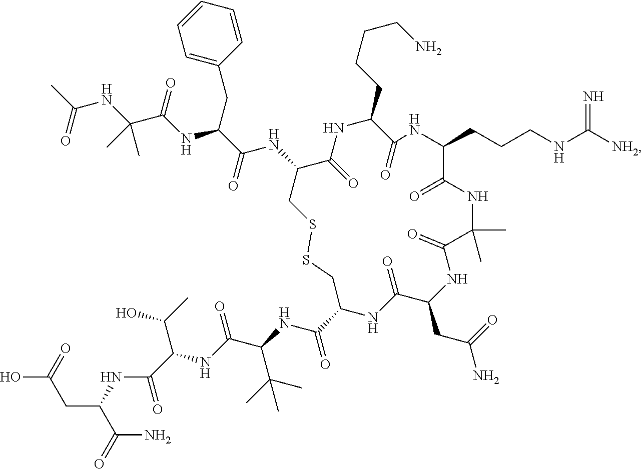 [SMILES: CC(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Aib-Pcf-[Cys-Lys-Arg-Aib-Asn-Cys]-NH₂ (PSM-0443) of the following formula

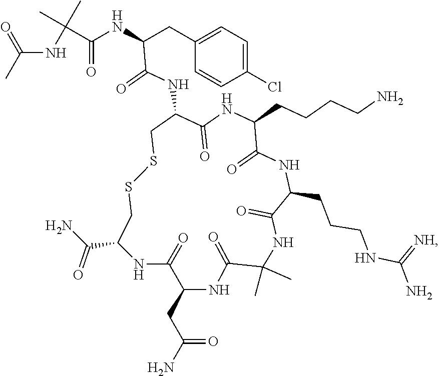 [SMILES: CC(=O)NC(C)(C)C(=O)N[C@@H](Cc1ccc(Cl)cc1)C(=O)N[C@H]1CSSC[C@@H](C(N)=O)NC(=O)[C@H](CC(N)=O)NC(=O)C(C)(C)NC(=O)[C@H](CCCNC(=N)N)NC(=O)[C@H](CCCCN)NC1=O]

compound Iva-Aib-Pcf-[Cys-Lys-Arg-Aib-Asn-Cys]-Tle-Thr-lys(DOTA)-NH₂ (PSM-0444) of the following formula

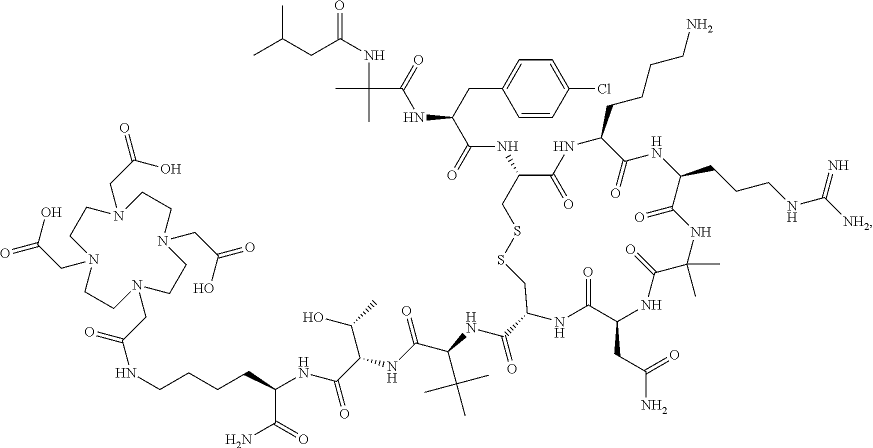 [SMILES: CC(C)CC(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Pcf-[Cys-Lys-Arg-Aib-Asn-Cys]-Tle-Thr-NH₂ (PSM-0445) of the following formula

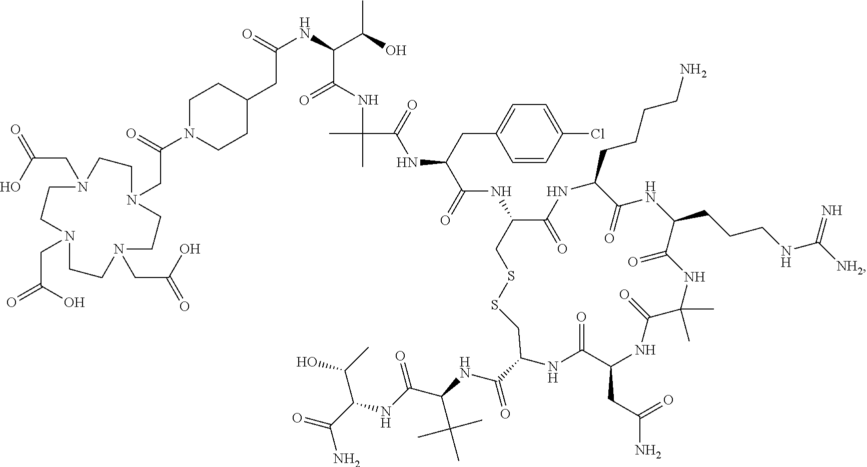 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Aib-Phe-[Cys-Lys-Arg-Aib-Asn-Cys]-Tle-Thr-NH₂ (PSM-0447) of the following formula

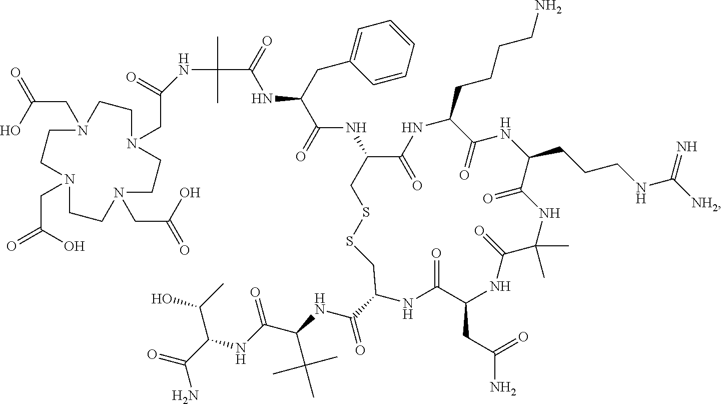 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Pab-Thr-Aib-Phe-[Cys-Lys-Arg-Aib-Asn-Cys]-Tle-Thr-NH₂ (PSM-0448) of the following formula

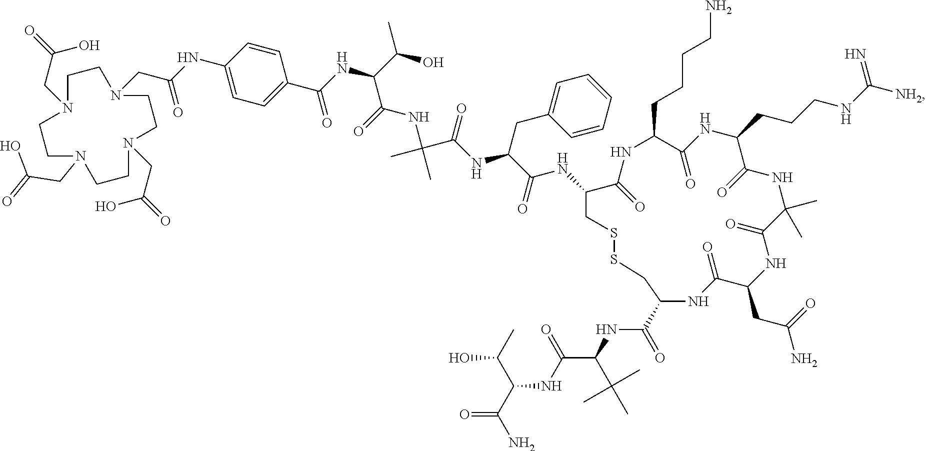 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)c2ccc(NC(=O)CN3CCN(CC(=O)O)CCN(CC(=O)O)CCN(CC(=O)O)CC3)cc2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pho-[Cys-Lys-Arg-Aib-Asn-Cys]-Tle-Bal-NH₂ (PSM-0449) of the following formula

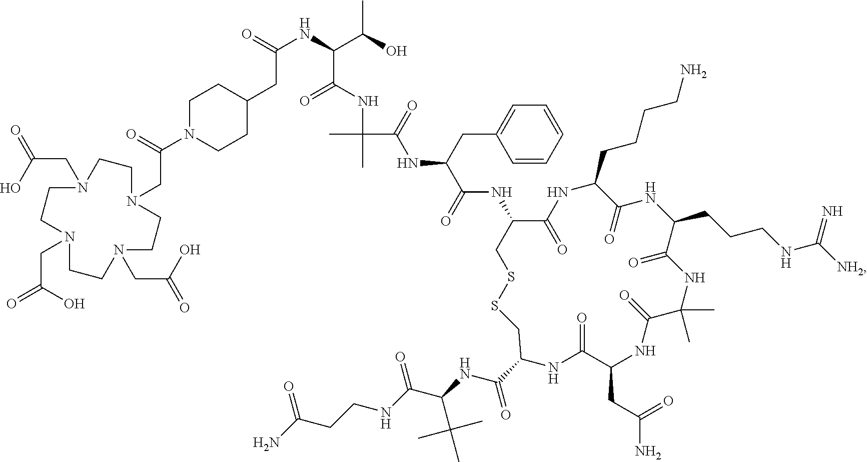 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccccc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Iva-Aib-Phe-[Cys-Lys-Arg-Aib-Asn-Cys]-Tle-Thr-NH₂ (PSM-0450) of the following formula

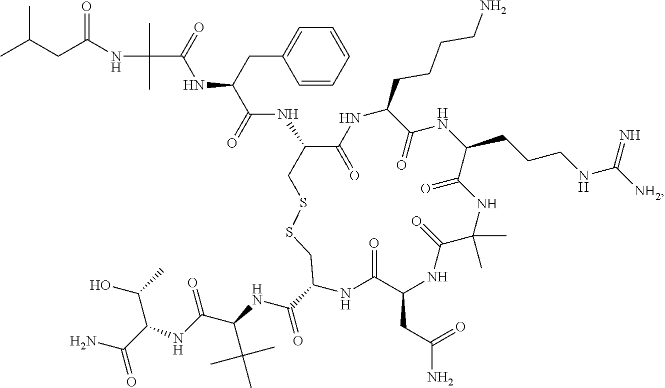 [SMILES: CC(C)CC(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Nmk-Arg-Aib-Asn-Cys]-Tle-Thr-NH₂ (PSM-0451) of the following formula

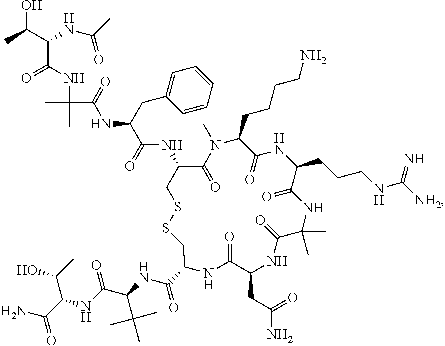 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)N(C)C1=O)[C@@H](C)O]

compound nBuCAyl-Thr-Aib-Pcf-[Cys-Lys-Arg-Aib-Asn-Cys]-Tle-Thr-lys (DOTA-Cmp)-NH₂ (PSM-0452) of the following formula

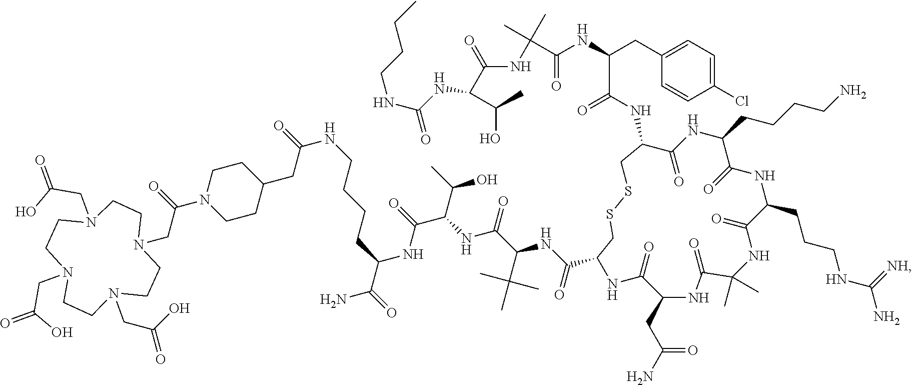 [SMILES: CCCCNC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Leu-NH₂ (PSM-0453) of the following formula

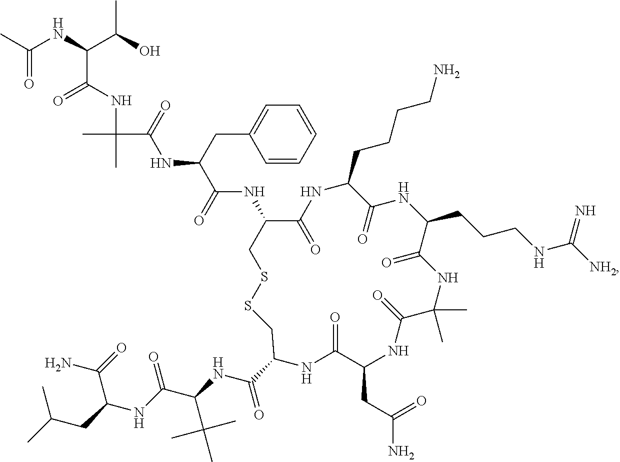 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@@H](CC(C)C)C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Ppa-[Cys-Lys-Cit-Aib-Asn-Cys]-Tle-Thr-NH₂ (PSM-0454) of the following formula

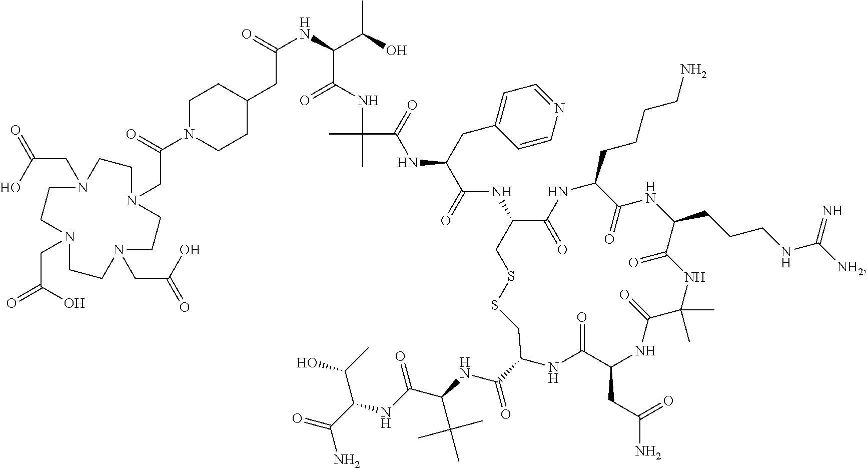 [SMILES: C[C@@H](O)[C@H](NC(=O)[C@@H](NC(=O)[C@@H]1CSSC[C@H](NC(=O)[C@H](Cc2ccncc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Nmt-Ala-Pcf-[Cys-Lys-Arg-Aib-Asn-Cys]-Tle-Thr-NH₂ (PSM-0455) of the following formula

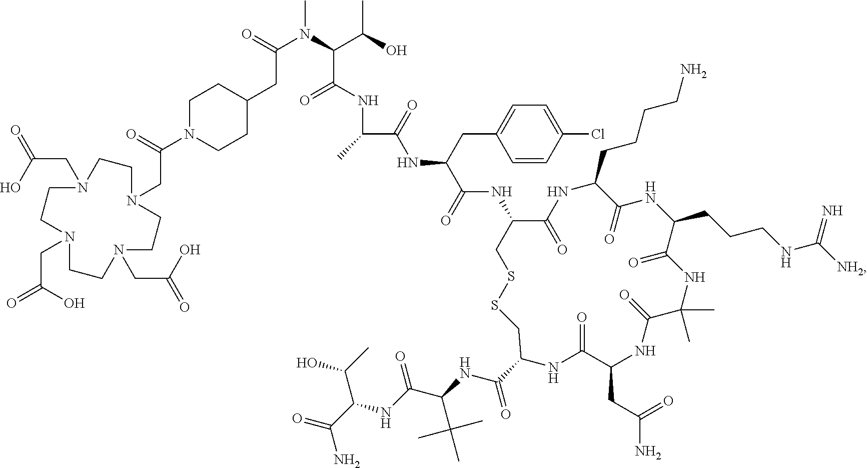 [SMILES: C[C@H](NC(=O)[C@H]([C@@H](C)O)N(C)C(=O)CC1CCN(C(=O)CN2CCN(CC(=O)O)CCN(CC(=O)O)CCN(CC(=O)O)CC2)CC1)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Pcf-[Cys-Lys-Arg-Aib-Asn-Cys]-Tle-en (Me) (PSM-0456) of the following formula

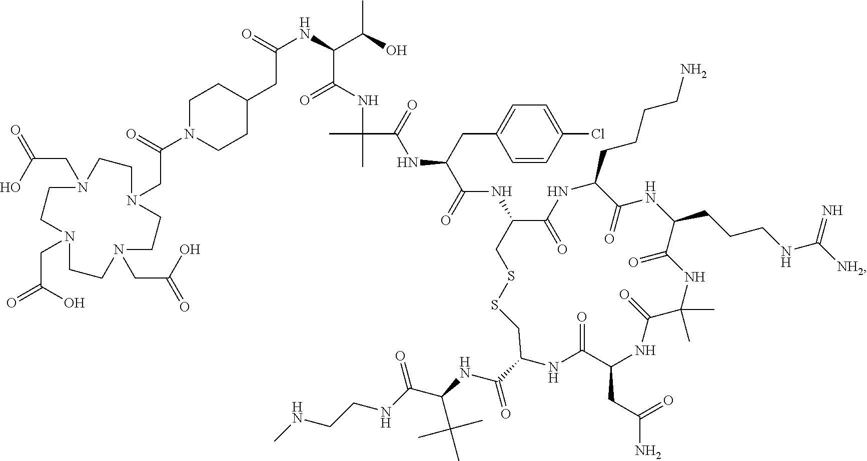 [SMILES: CNCCNC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C]

compound DOTA-Pamb-Aib-Phe-[Cys-Lys-Arg-Aib-Asn-Cys]-Tle-NH₂ (PSM-0458) of the following formula

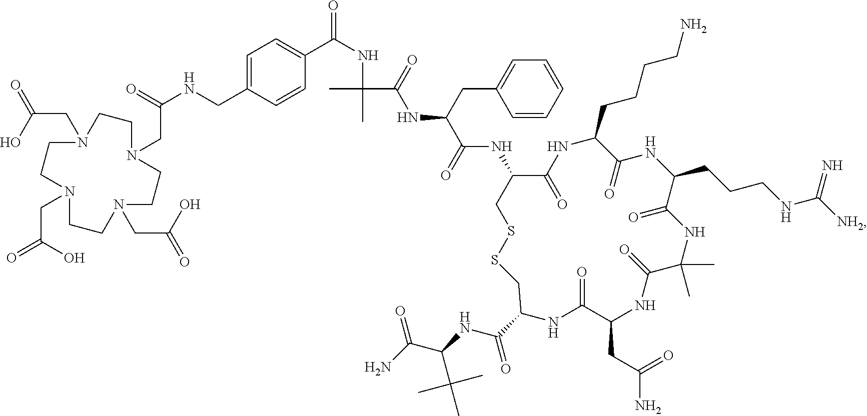 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccccc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound DOTA-Cmp-Thr-Aib-Mnf-[Cys-Lys(Me)-Arg(Me)-Aib-Asn-Cys]-Tle-NH₂ (PSM-0459) of the following formula

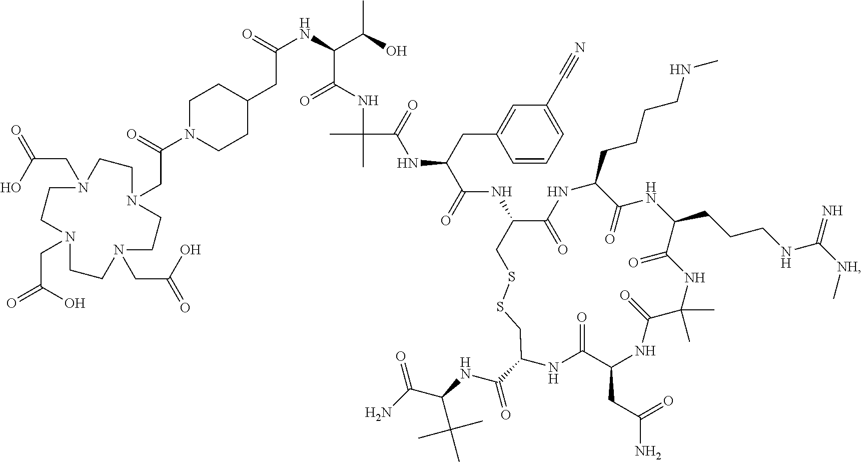 [SMILES: CNCCCC[C@@H]1NC(=O)[C@@H](NC(=O)[C@H](Cc2cccc(C#N)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Ala-Phe-[Cys-Lys-Arg-Pro-Asn-Cys]-Nle-Thr-Asp-NH₂ (PSM-0460) of the following formula

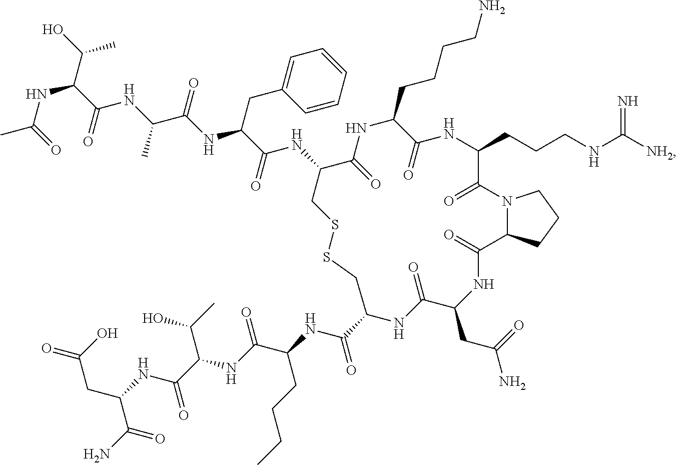 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N2CCC[C@H]2C(=O)N[C@@H](CC(N)=O)C(=O)N1)C(=O)N[C@H](C(=O)N[C@@H](CC(=O)O)C(N)=O)[C@@H](C)O]

compound Hib-Pcf-[Cys-Lys-Arg-Aib-Asn-Cys]-Tle-Thr-NH₂ (PSM-0461) of the following formula

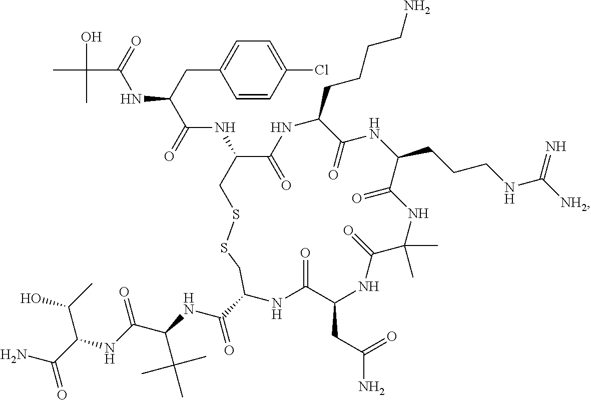 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Hex-Thr-Aib-Phe-[Cys-Lys-Arg-Ala-Asn-Cys]-Tle-Thr-Ttds-lys(DOTA)-NH₂ (PSM-0462) of the following formula

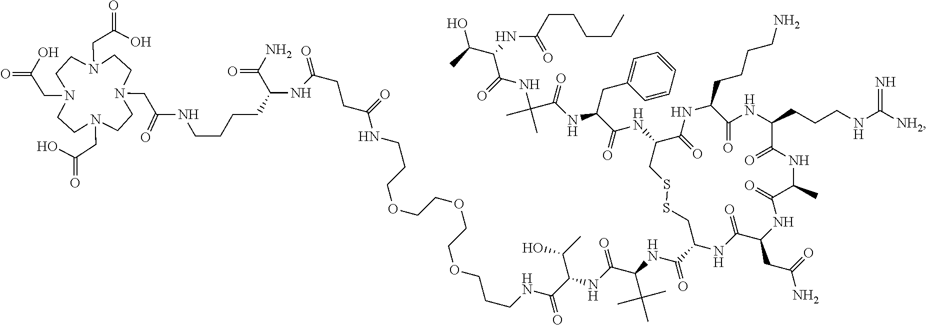 [SMILES: CCCCCC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys(DOTA-APAc)-Asn-Cys]-Tle-Thr-NH₂ (PSM-0464) of the following formula

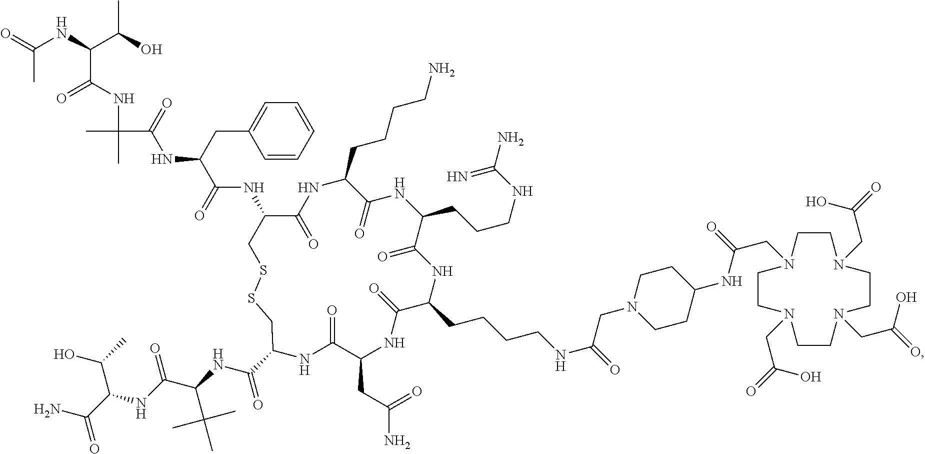 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CN2CCC(NC(=O)CN3CCN(CC(=O)O)CCN(CC(=O)O)CCN(CC(=O)O)CC3)CC2)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys(DOTA-Ttds)-Asn-Cys]-Tle-Thr-NH₂ (PSM-0465) of the following formula

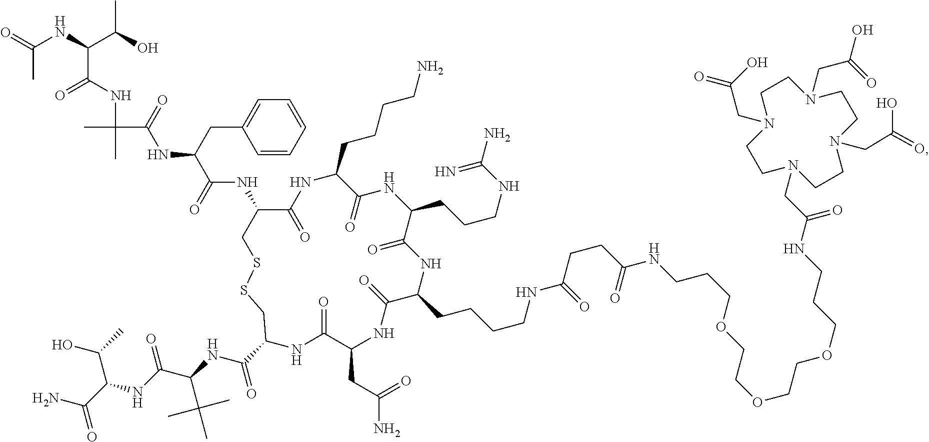 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CCC(=O)NCCCOCCOCCOCCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys(Ac)-Arg-Aib-Asn-Cys]-Tle-Thr-NH₂ (PSM-0466) of the following formula

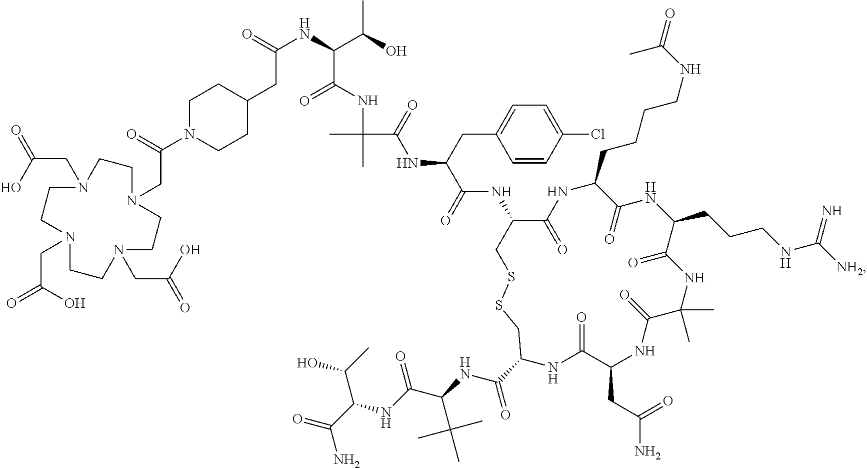 [SMILES: CC(=O)NCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC1=O]

compound DOTA-Cmp-Thr-Aib-Pcf-[Cys-Lys-Arg-Aib-Asn-Cys]-Tle-Bal-NH₂ (PSM-0467) of the following formula

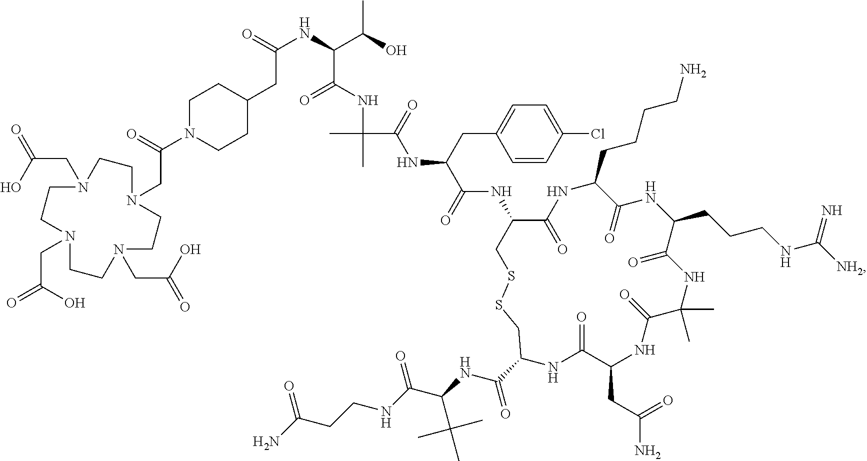 [SMILES: C[C@@H](O)[C@H](NC(=O)CC1CCN(C(=O)CN2CCN(CC(=O)O)CCN(CC(=O)O)CCN(CC(=O)O)CC2)CC1)C(=O)NC(C)(C)C(=O)N[C@@H](Cc1ccc(Cl)cc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound EuDOTA-Cmp-Thr-Aib-Pcf-[Cys-Lys-Arg-Aib-Asn-Cys]-Tle-Thr-NH₂ (PSM-0468) of the following formula

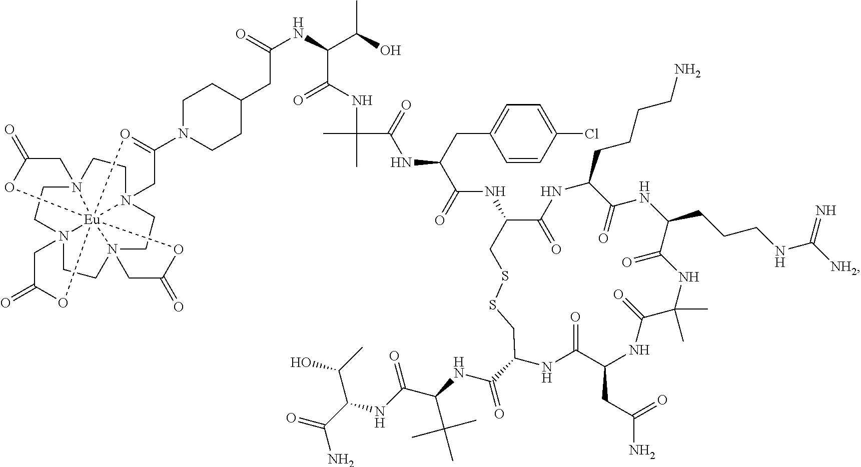

compound Ac-Ser-Ala-Phe-[Cys-Lys-Arg-Ala-Asn-Cys]-Nle-Thr-Asp-NH₂ (PSM-0469) of the following formula

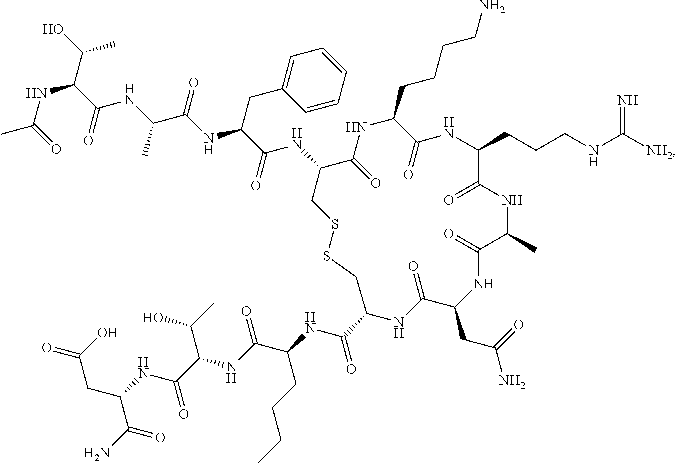 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound nBuCAyl-Thr-Aib-Mcf-[Cys-Lys-Arg-Aib-Asn-Cys]-Tle-Thr-Cmp-lys(DOTA)-NH₂ (PSM-0470) of the following formula

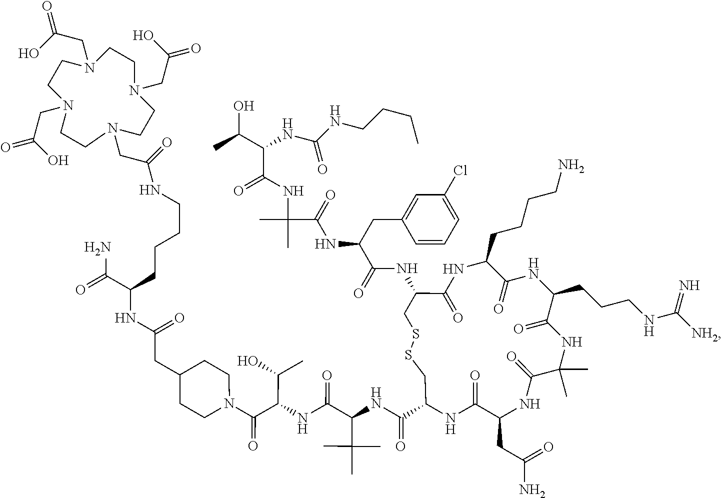 [SMILES: CCCCNC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Thr-Pab-lys(DOTA)-NH₂ (PSM-0471) of the following formula

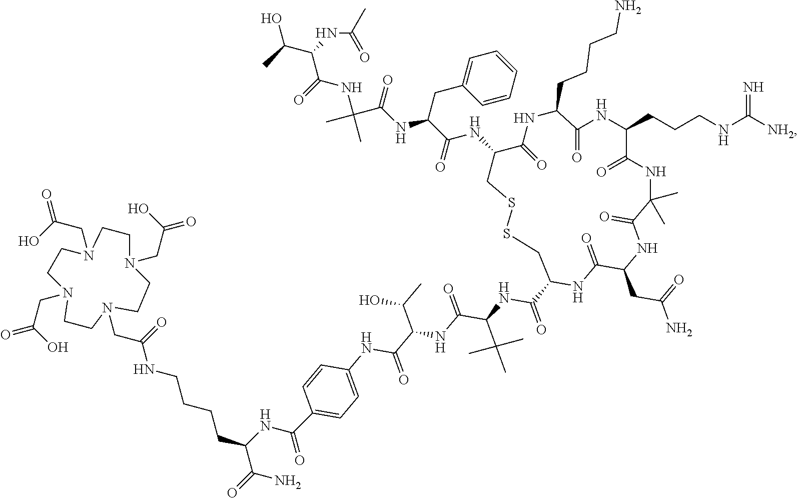 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)Nc2ccc(C(=O)N[C@H](CCCCNC(=O)CN3CCN(CC(=O)O)CCN(CC(=O)O)CCN(CC(=O)O)CC3)C(N)=O)cc2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Pcf-[Cys-Lys(Me)-Arg-Aib-Asn-Cys]-Tle-en-DOTA (PSM-0472) of the following formula

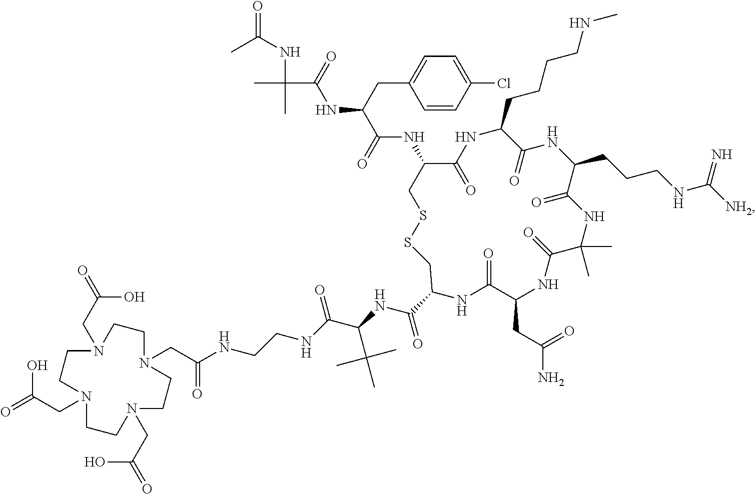 [SMILES: CNCCCC[C@@H]1NC(=O)[C@@H](NC(=O)[C@H](Cc2ccc(Cl)cc2)NC(=O)C(C)(C)NC(C)=O)CSSC[C@@H](C(=O)N[C@H](C(=O)NCCNC(=O)CN2CCN(CC(=O)O)CCN(CC(=O)O)CCN(CC(=O)O)CC2)C(C)(C)C)NC(=O)[C@H](CC(N)=O)NC(=O)C(C)(C)NC(=O)[C@H](CCCNC(=N)N)NC1=O]

compound Ac-Thr-Ala-Phe-[Cys-Lys-Arg-ala-Asn-Cys]-Nle-Thr-Asp-NH₂ (PSM-0474) of the following formula

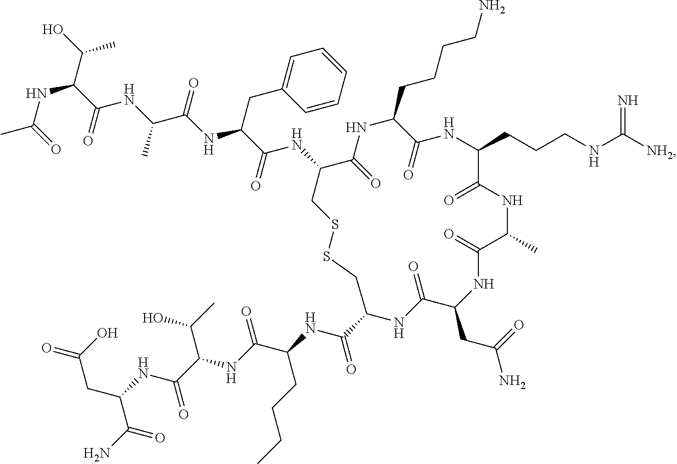 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Ala-Phe-[Cys-Ala-Arg-Ala-Ass-Cys]-Nle-Thr-Asp-NH₂ (PSM-0475) of the following formula

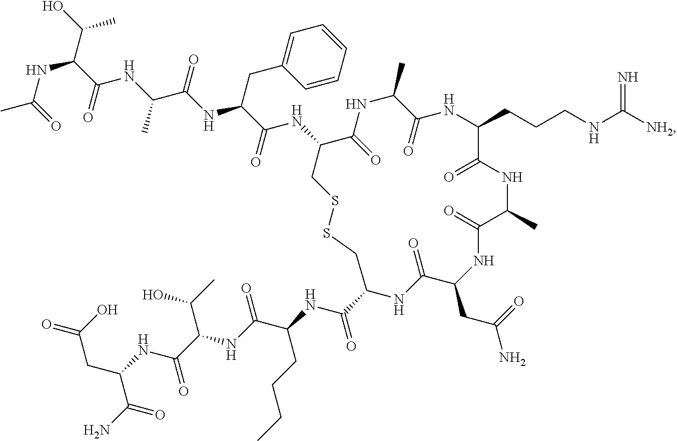 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](C)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Als-Phe-[Cys-Lys-Arg-Ala-Asn-Cys]-Nle-Als-Asp-NH₂ (PSM-0476) of the following formula

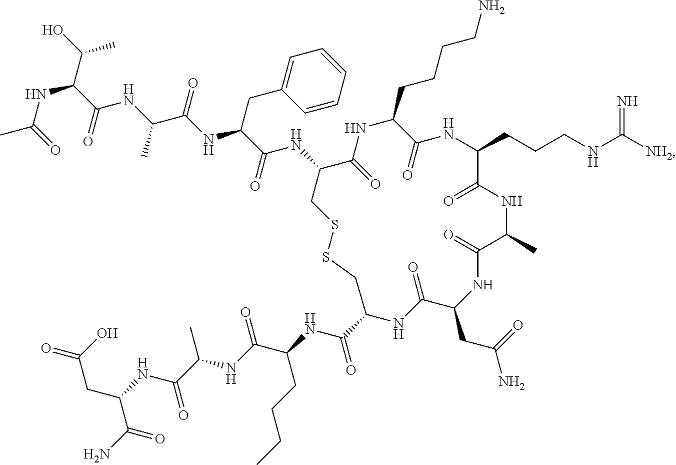 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@@H](C)C(=O)N[C@@H](CC(=O)O)C(N)=O]

compound Ac-Thr-Aib-Phe-[Cys-Lys-Arg-Aib-Asn-Cys]-Tle-Gly-NH₂ (PSM-0477) of the following formula

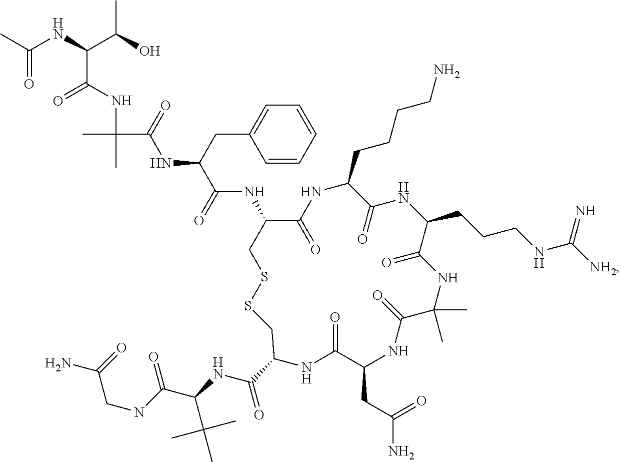 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)NCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asp-Cys]-Tle-Thr-NH₂ (PSM-0478) of the following formula

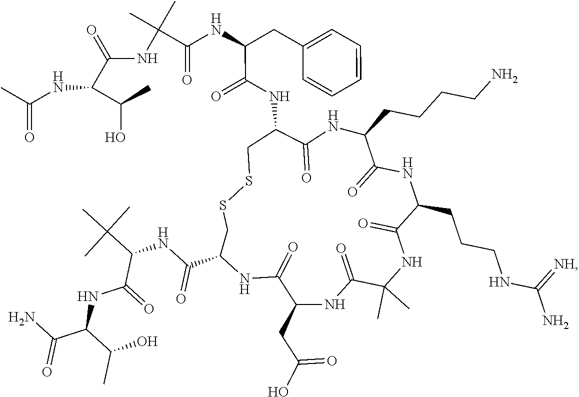 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(=O)O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Gly-Cys]-Tle-Thr-NH₂ (PSM-0479) of the following formula

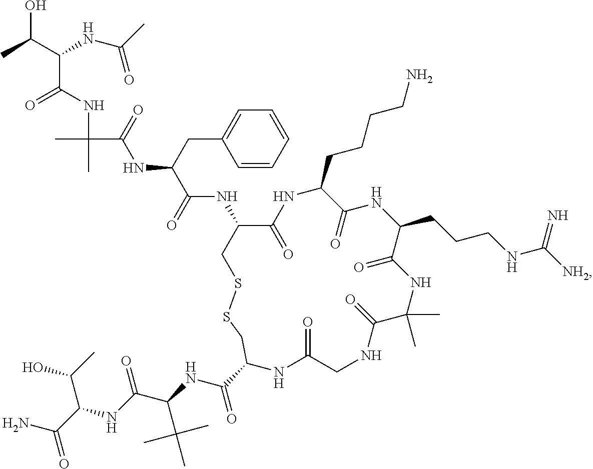 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)CNC(=O)C(C)(C)NC(=O)[C@H](CCCNC(=N)N)NC(=O)[C@H](CCCCN)NC1=O)[C@@H](C)O]

compound DOTA-Ttds-Thr-Ala-Phe-[Cys-Lys-Arg-Ala-Asn-Cys]-Lou-NH₂ (PSM-0480) of the following formula

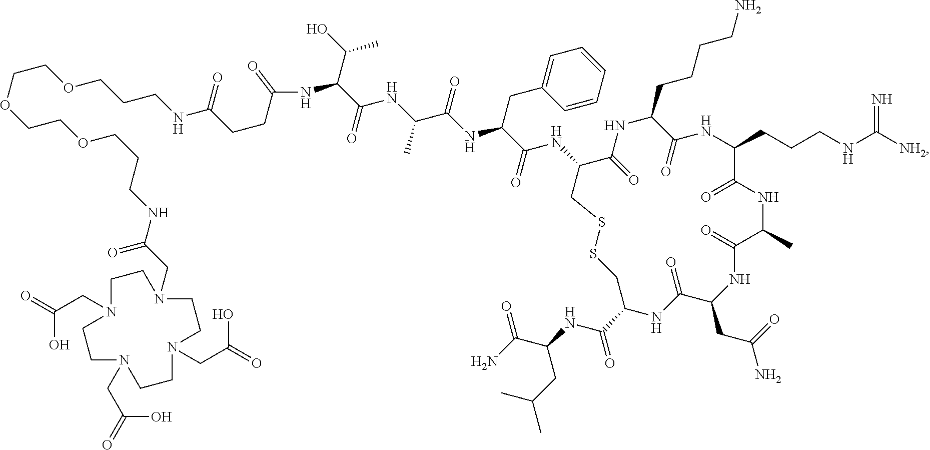 [SMILES: CC(C)C[C@H](NC(=O)[C@@H]1CSSC[C@H](NC(=O)[C@H](Cc2ccccc2)NC(=O)[C@H](C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(N)=O]

compound Ac-Aib-Pcf-[Cys-Lys(Me)-Arg(Me)-Aib-Asn-Cys]-Tle-en-DOTA (PSM-0481) of the following formula

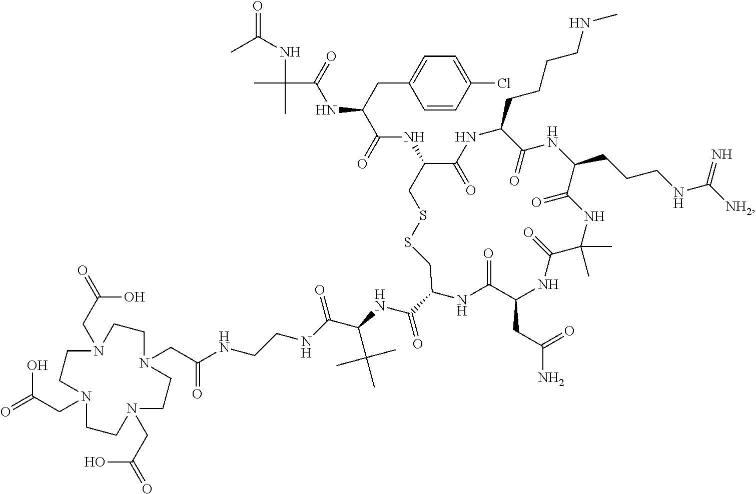 [SMILES: CNCCCC[C@@H]1NC(=O)[C@@H](NC(=O)[C@H](Cc2ccc(Cl)cc2)NC(=O)C(C)(C)NC(C)=O)CSSC[C@@H](C(=O)N[C@H](C(=O)NCCNC(=O)CN2CCN(CC(=O)O)CCN(CC(=O)O)CCN(CC(=O)O)CC2)C(C)(C)C)NC(=O)[C@H](CC(N)=O)NC(=O)C(C)(C)NC(=O)[C@H](CCCNC(=N)N)NC1=O]

compound Ac-Thr-Aib-Pcf-[Cys-Lys-Arg-Aib-Asn-Cys]-Tle-Thr-Cmp-lys(DOTA)-NH₂ (PSM-0482) of the following formula

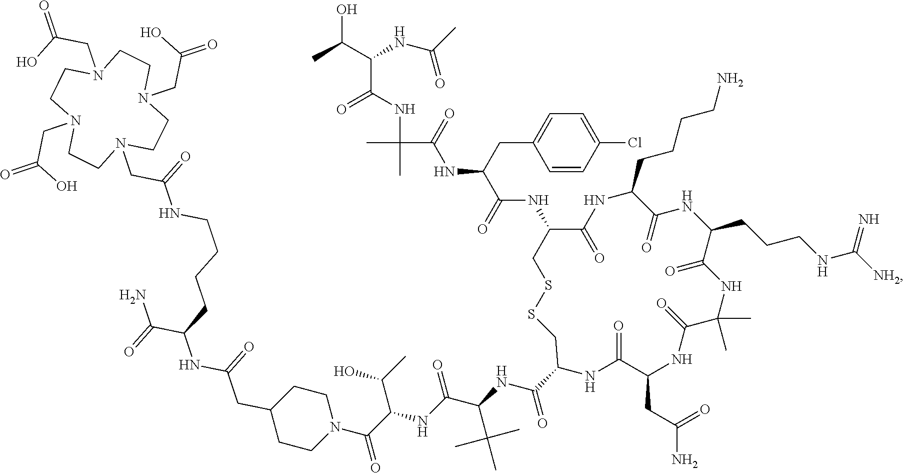 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-KMe3-Arg(Me)-Aib-Asn-Cys]-Tle-Thr-NH₂ (PSM-0483) of the following formula

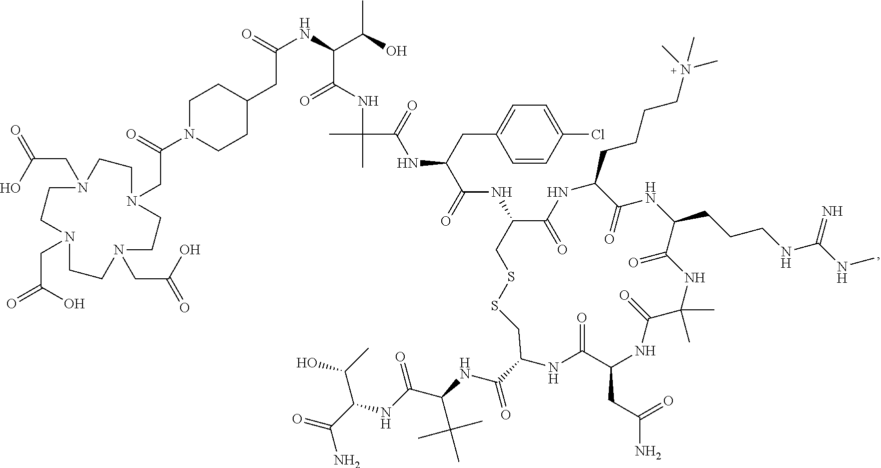

compound nBuCAyl-Thr-Aib-Phe-[Cys-Lys-Arg-Aib-Asn-Cys]-Tle-Thr-Cmp-lys(DOTA)-NH₂ (PSM-0484) of the following formula

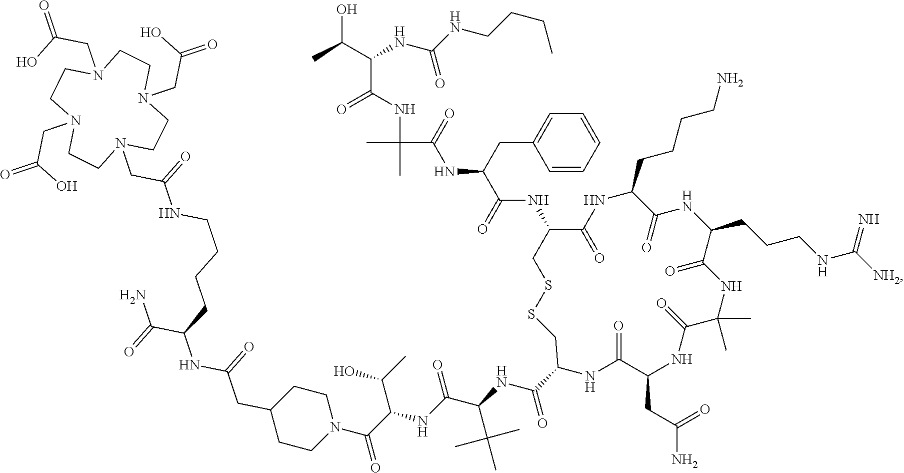 [SMILES: CCCCNC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Hgn-Arg-Aib-Asn-Cys]-Tle-Thr-NH₂ (PSM-0485) of the following formula

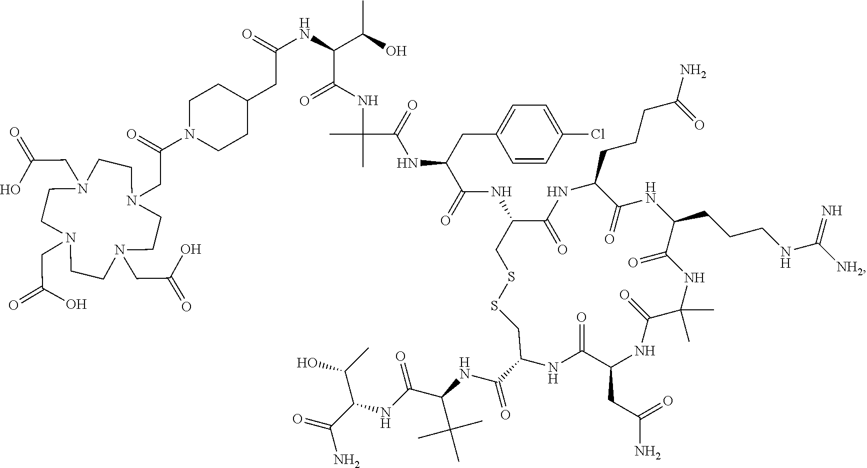 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCC(N)=O)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Mcf-[Cys-Lys-Arg-Aib-Asn-Cys]-Tle-Thr-Cmp-lys(DOTA)-NH₂ (PSM-0486) of the following formula

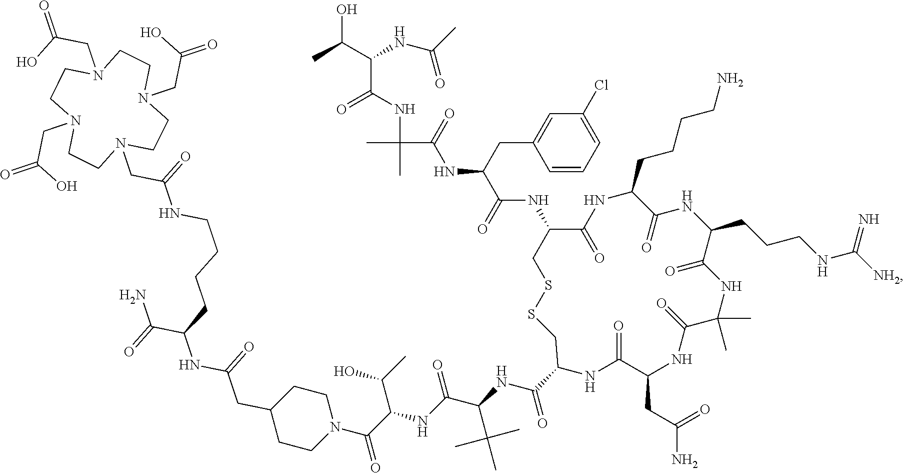 [SMILES: CC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Tyr-[Cys-Lys-Arg-Aib-Asn-Cys]-Tle-Thr-Asp-NH₂ (PSM-0488) of the following formula

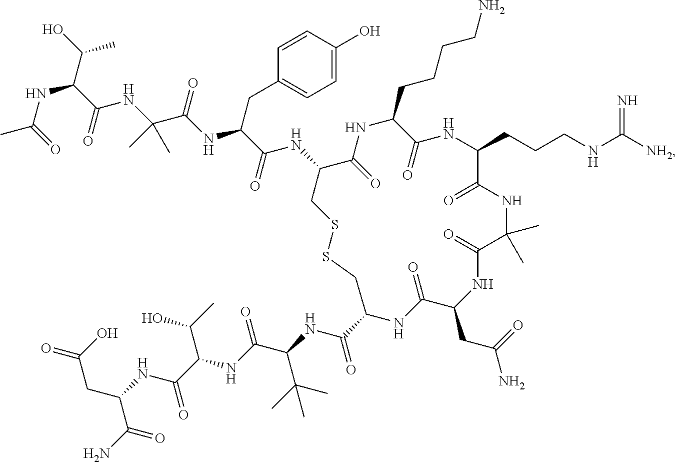 [SMILES: CC(=O)N[C@H](C(=O)NC(C)(C)C(=O)N[C@@H](Cc1ccc(O)cc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound DOTA-Cmp-Aib-Pcf-[Cys-Lys-Gin (Gu)-Aib-Asn-Cys]-Tle-Thr-NH₂ (PSM-0489) of the following formula

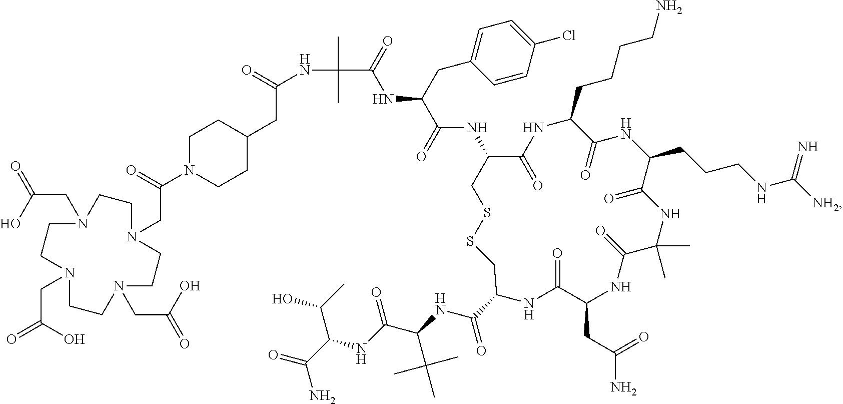 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CC2CCN(C(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys (Bio)-Arg-Aib-Asn-Cys]-Tle-Thr-NH₂ (PSM-0490) of the following formula

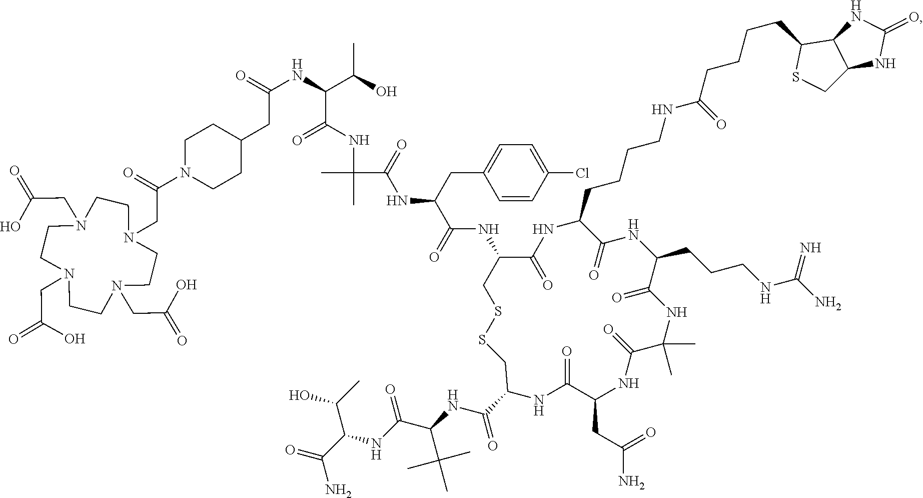 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCNC(=O)CCCC[C@@H]2SC[C@@H]3NC(=O)N[C@@H]32)C(=O)N[C@@H](CCCNC(=N)N)C(=O)NC(C)(C)C(=O)N[C@@H](CC(N)=O)C(=O)N1)C(C)(C)C)C(N)=O]

compound Ac-Ala-Ala-Phe-[Cys-Lys-Arg-Ala-Asn-Cys]-Nle-Thr-Asp-NH₂ (PSM-0491) of the following formula

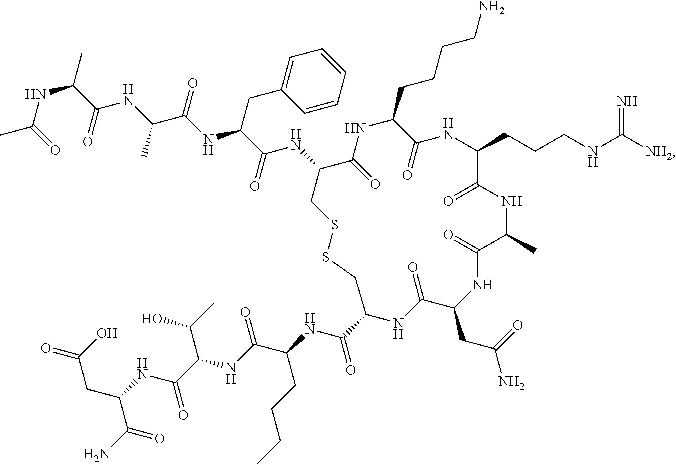 [SMILES: CCCC[C@H](NC(=O)[C@@H]1CSSC[C@H](NC(=O)[C@H](Cc2ccccc2)NC(=O)[C@H](C)NC(=O)[C@H](C)NC(C)=O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound DOTA-Cmp-Thr-Aib-Phe-[Cys-Lys-Arg-Aib-Asn-Cys]-Tle-Thr-NH₂ (PSM-0492) of the following formula

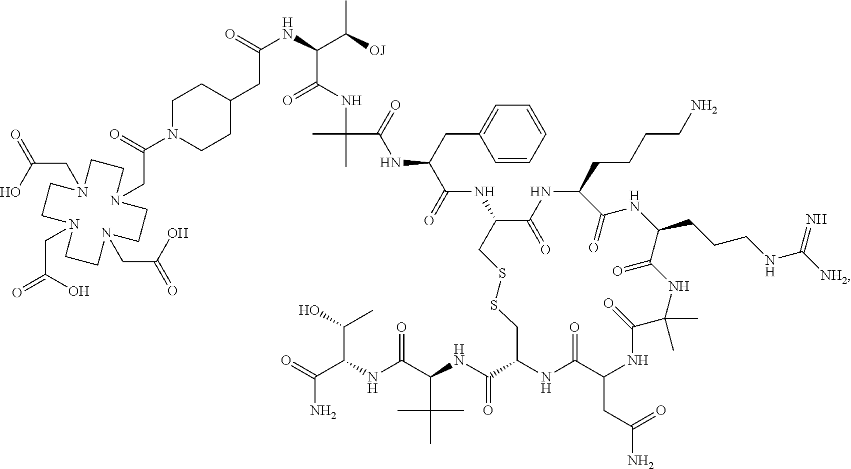

compound Ac-Thr-Aib-Pcf-[Cys-Lys-Arg-Aib-Asn-Cys]-Tle-Thr-OH (PSM-0493) of the following formula

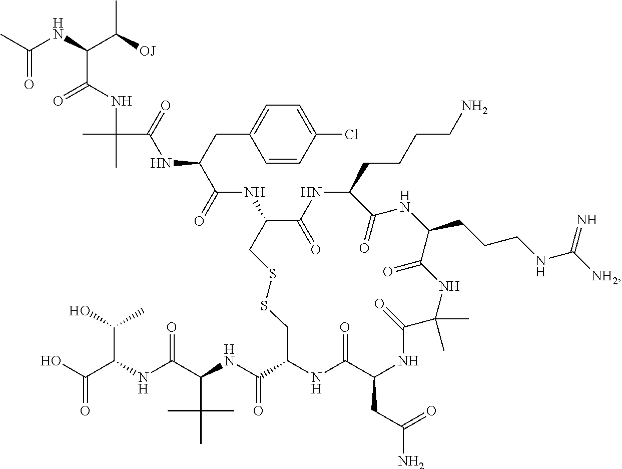

compound Ac-Aib-Pcf-[Cys-Lys-Gin (Gu)-Aib-Asn-Cys]-Tle-Thr-Cmp-lys(DOTA)-NH₂ (PSM-0494) of the following formula

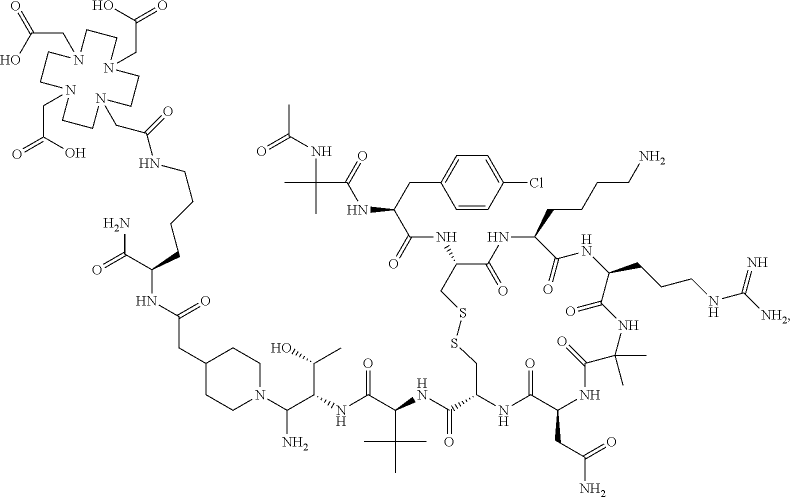 [SMILES: CC(=O)NC(C)(C)C(=O)N[C@@H](Cc1ccc(Cl)cc1)C(=O)N[C@H]1CSSC[C@@H](C(=O)N[C@H](C(=O)N[C@H](C(N)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Crown-Cmp-Thr-Aib-Pcf-[Cys-Lys(Me)-Arg(Me)-Aib-Asn-Cys]-Tle-Thr-NH₂ (PSM-0495) of the following formula

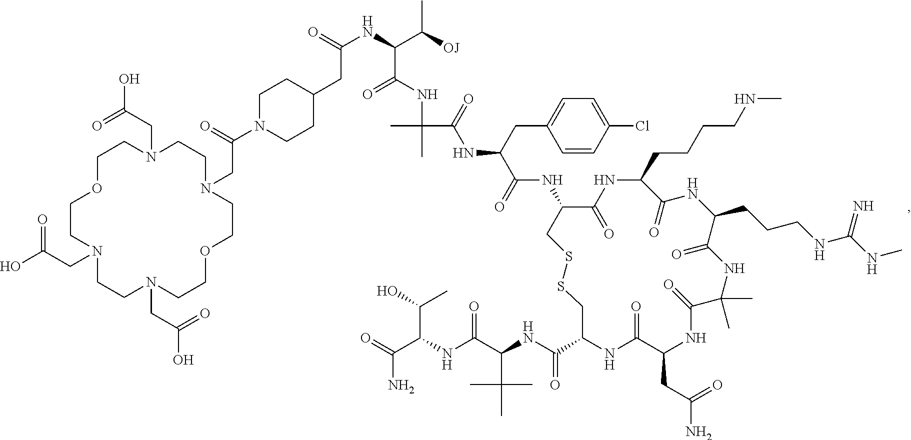

compound Ac-Thr-Aib-Pcf-[Cys-Lys-Cit-Aib-Glu-Cys]-Tle-Thr-NH₂ (PSM-0496) of the following formula

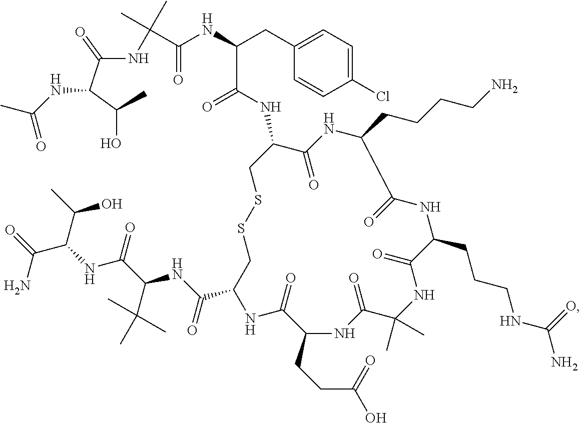 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CCC(=O)O)NC(=O)C(C)(C)NC(=O)[C@H](CCCNC(N)=O)NC(=O)[C@H](CCCCN)NC1=O)[C@@H](C)O]

compound DOTA-Cmp-Thr-Aib-Pcf-[Cys-Lys-Arg-Aib-Asn-Cys]-Nml-Thr-NH₂ (PSM-0497) of the following formula

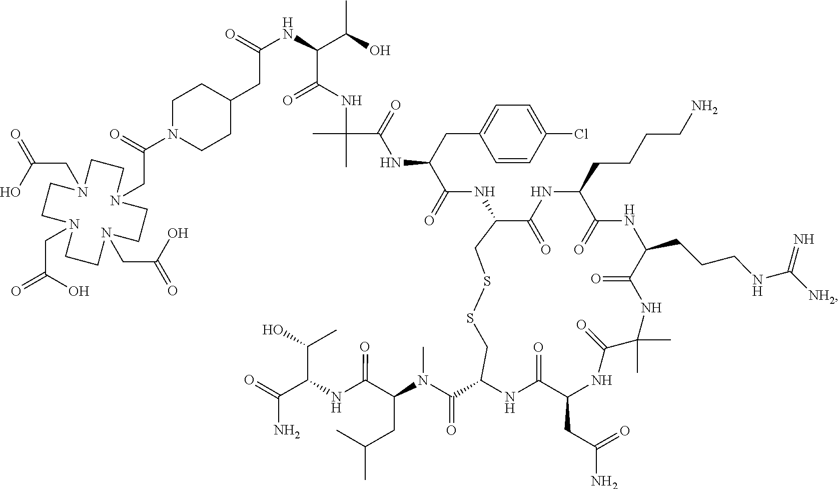 [SMILES: CC(C)C[C@@H](C(=O)N[C@H](C(N)=O)[C@@H](C)O)N(C)C(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1]

compound H-Met-Thr-Ala-Phe-[Cys-Lys-Arg-Ala-Asn-Cys]-Lou-Thr-Asp-Gly-Ser-NH₂ (PSM-0498) of the following formula

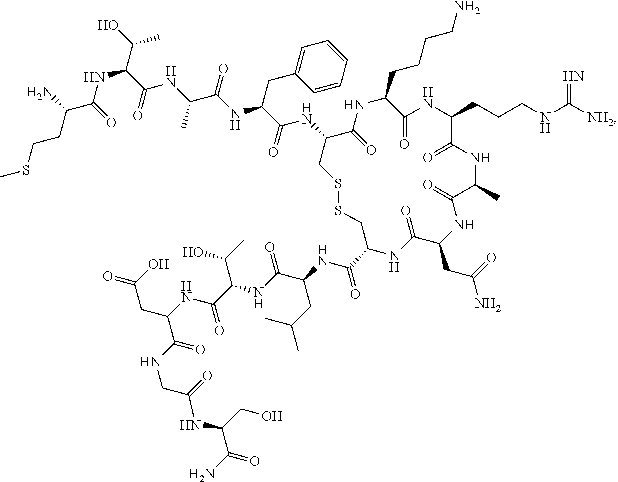 [SMILES: CSCC[C@H](N)C(=O)N[C@H](C(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)NC(CC(=O)O)C(=O)NCC(=O)N[C@@H](CO)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys(Me)-Egd-Aib-Asn-Cys]-Tle-Thr-NH₂ (PSM-0499) of the following formula

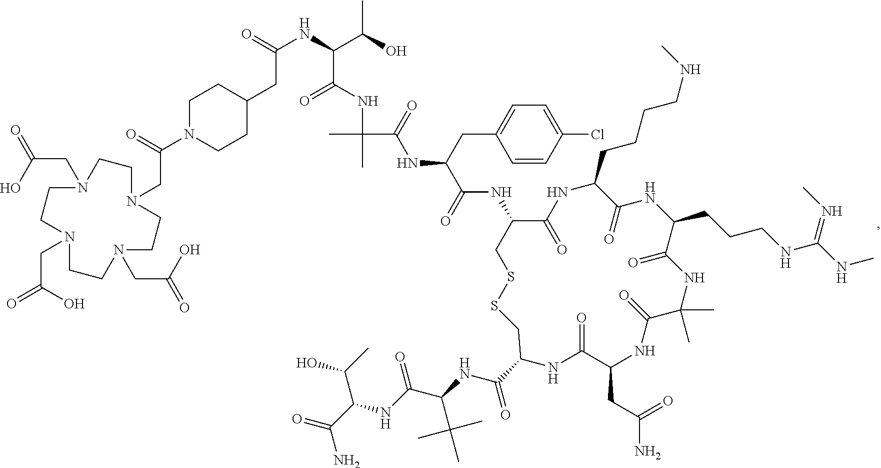 [SMILES: C/N=C(/NC)NCCC[C@@H]1NC(=O)[C@H](CCCCNC)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound nBuCAyl-Thr-Aib-Pcf-[Cys-Lys-Cit-Aib-Asn-Cys]-Tle-Thr-lys (DOTA-Cmp)-NH₂ (PSM-0500) of the following formula

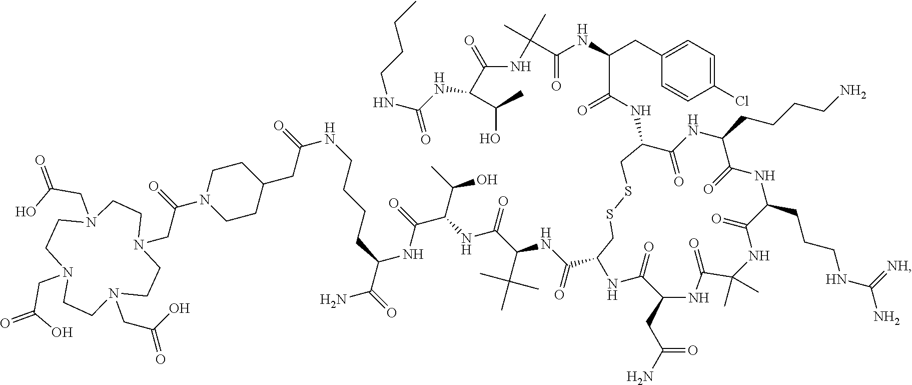 [SMILES: CCCCNC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(=O)N[C@H](CCCCNC(=O)CC2CCN(C(=O)CN3CCN(CC(=O)O)CCN(CC(=O)O)CCN(CC(=O)O)CC3)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Ala-Asn-Cys]-Nle-Thr-Asp-NH₂ (PSM-0501) of the following formula

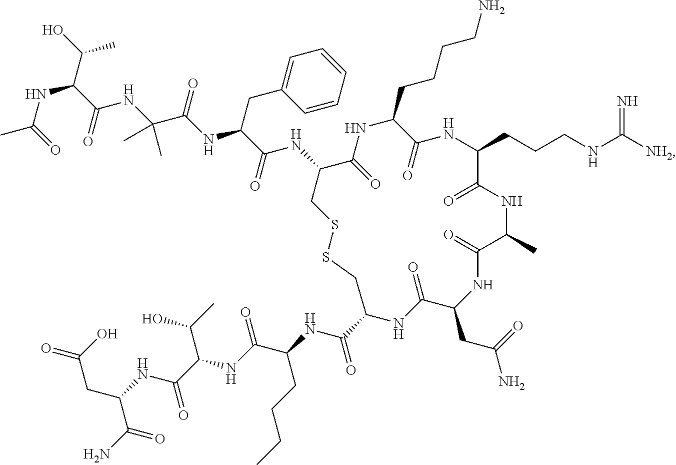 [SMILES: CCCC[C@H](NC(=O)[C@@H]1CSSC[C@H](NC(=O)[C@H](Cc2ccccc2)NC(=O)C(C)(C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Hex-Thr-Aib-Phe-[Cys-Lys-Arg-Ala-Asn-Cys]-Tle-Thr-Asp-Ttds-lys(DOTA)-NH₂ (PSM-0502) of the following formula

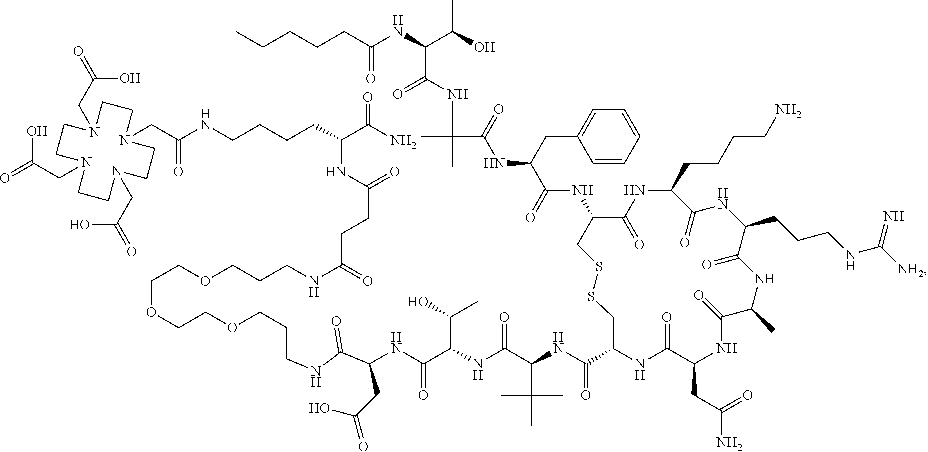 [SMILES: CCCCCC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Mmf-[Cys-Lys(Me)-Arg(Me)-Aib-Asn-Cys]-Tle-NH₂ (PSM-0503) of the following formula

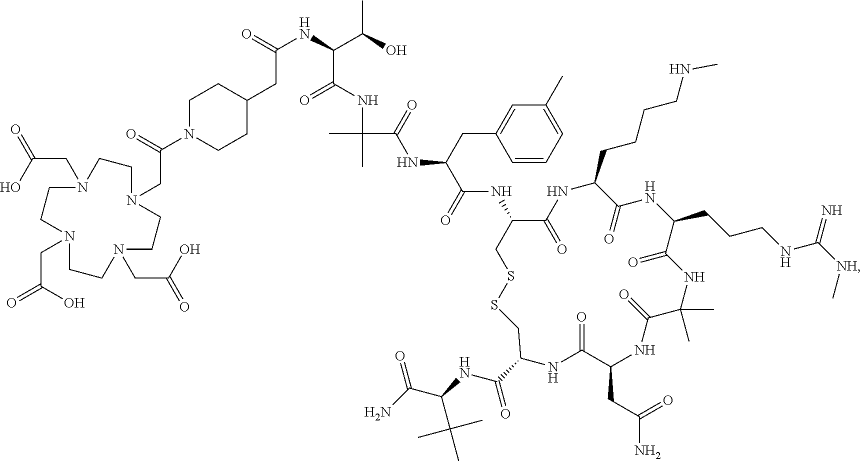 [SMILES: CNCCCC[C@@H]1NC(=O)[C@@H](NC(=O)[C@H](Cc2cccc(C)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound DOTA-Ttds-Thr-Ala-Phe-[Cys-Lys-Arg-Ala-Asn-Cys]-Nle-Thr-Asp-NH₂ (PSM-0504) of the following formula

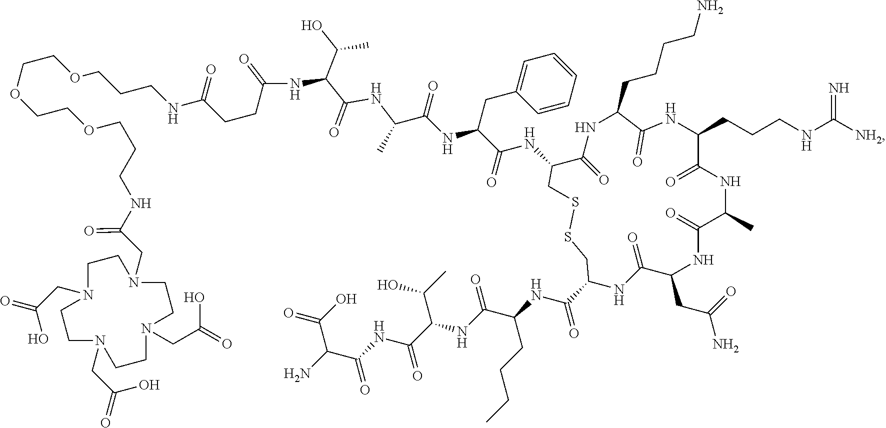

compound DOTA-020c-Thr-Aib-Phe-[Cys-Lys-Arg-Aib-Asn-Cys]-Tle-Thr-NH₂ (PSM-0505) of the following formula

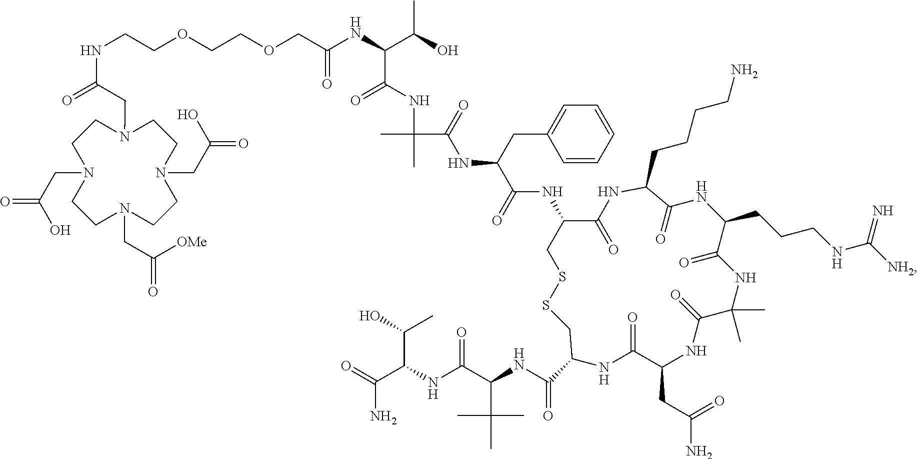 [SMILES: COC(=O)CN1CCN(CC(=O)O)CCN(CC(=O)NCCOCCOCC(=O)N[C@H](C(=O)NC(C)(C)C(=O)N[C@@H](Cc2ccccc2)C(=O)N[C@H]2CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC2=O)[C@@H](C)O)CCN(CC(=O)O)CC1]

compound Ac-Aib-Pho-[Cys-Lys-Arg-Ala-Asn-Cys]-Tle-Thr-Asp-NH₂ (PSM-0506) of the following formula

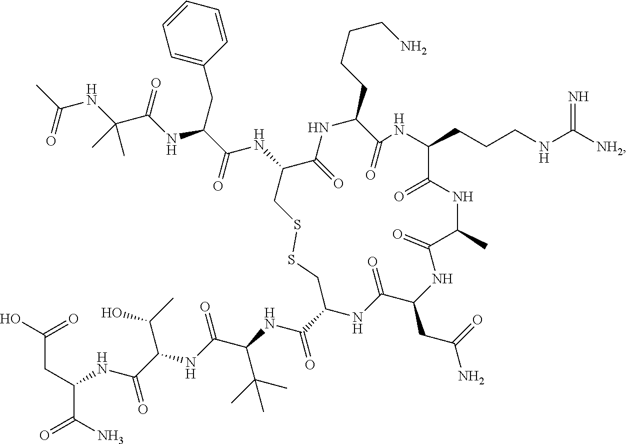 [SMILES: CC(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O]

compound DOTA-Cmp-Aib-Pho-[Cys-Lys-Arg-Aib-Asn-Cys]-Tle-Thr-NH₂ (PSM-0507) of the following formula

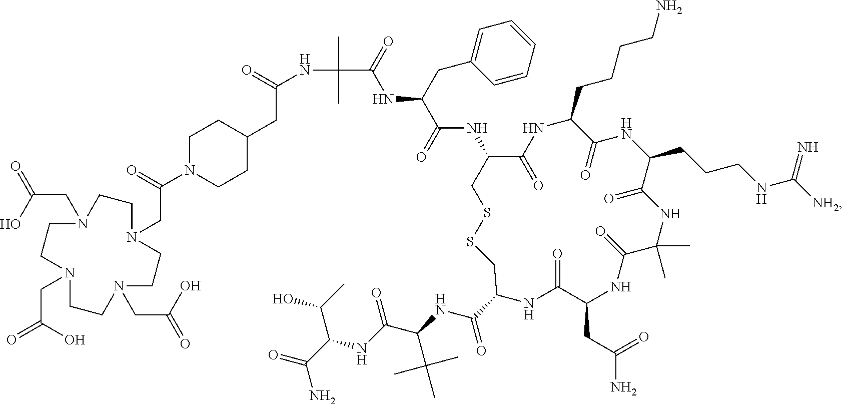 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CC2CCN(C(=O)CN3CCN(CC(=O)O)CCN(CC(=O)O)CCN(CC(=O)O)CC3)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Pam-Phe-[Cys-Lys-Arg-Aib-Asn-Cys]-Tle-Thr-NH₂ (PSM-0508) of the following formula

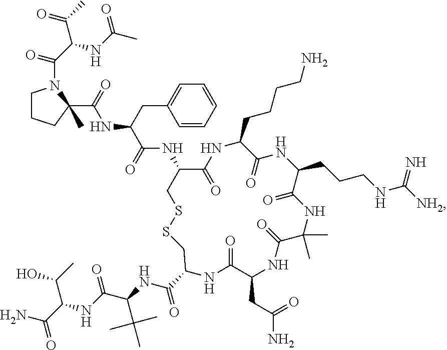

compound HPA-Aib-Pcf-[Cys-Lys-Arg-Aib-Asn-Cys]-Tle-Thr-Cmp-lys(DOTA)-NH₂ (PSM-0509) of the following formula

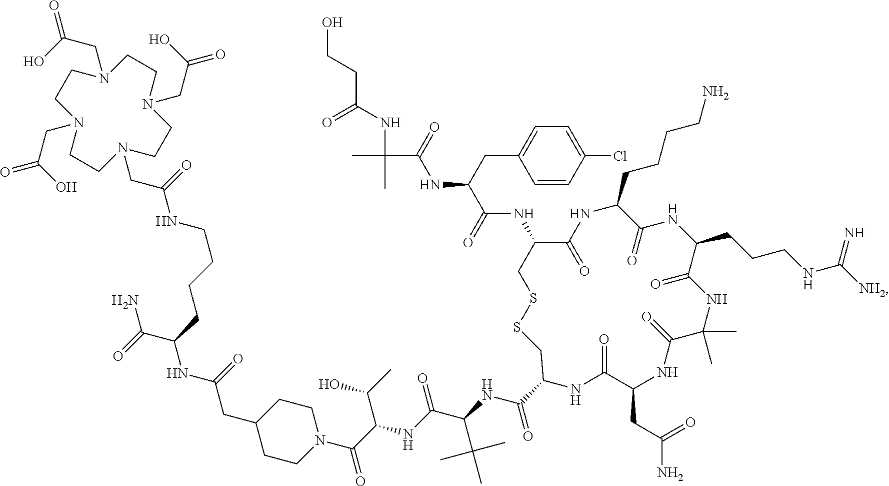 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)CCO)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(=O)N1CCC(CC(=O)N[C@H](CCCCNC(=O)CN2CCN(CC(=O)O)CCN(CC(=O)O)CCN(CC(=O)O)CC2)C(N)=O)CC1]

compound Ac-Thr-Aib-Phe-[Cys-Lys-Arg-Aib-Asn-Cys]-Tle-NHBu (PSM-0510) of the following formula

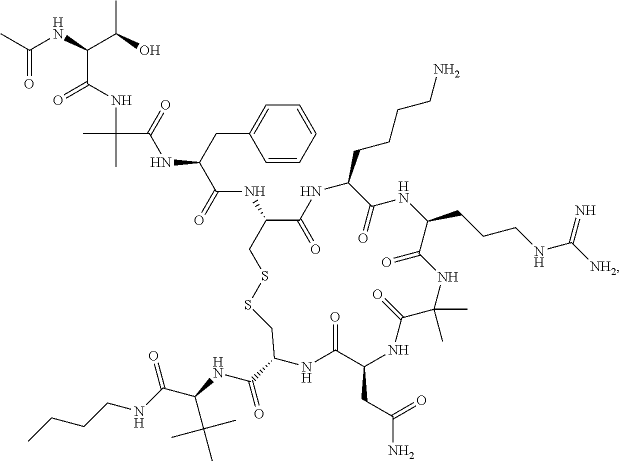 [SMILES: CCCCNC(=O)[C@@H](NC(=O)[C@@H]1CSSC[C@H](NC(=O)[C@H](Cc2ccccc2)NC(=O)C(C)(C)NC(=O)[C@@H](NC(C)=O)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C]

compound DOTA-Cmp-Thr-Aib-Pcf-[Cys-Om-Arg-Aib-Asn-Cys]-Tle-Thr-NH₂ (PSM-0511) of the following formula

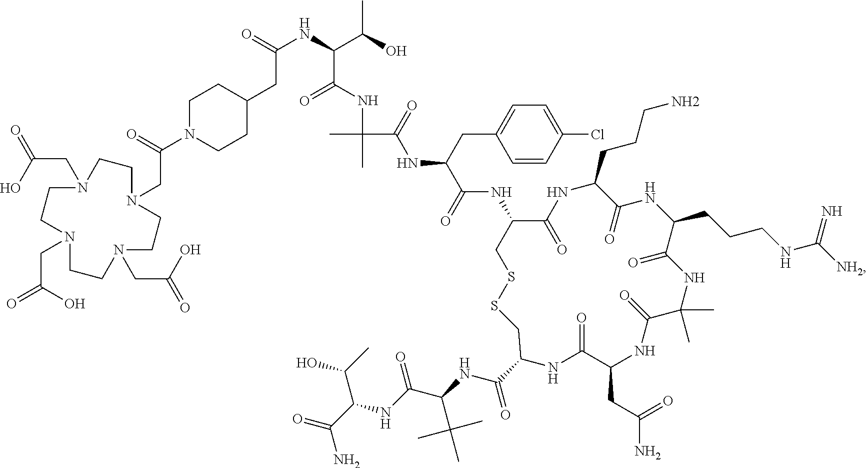 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Opa-[Cys-Lys-Cit-Aib-Asn-Cys]-Tle-Thr-NH₂ (PSM-0512) of the following formula

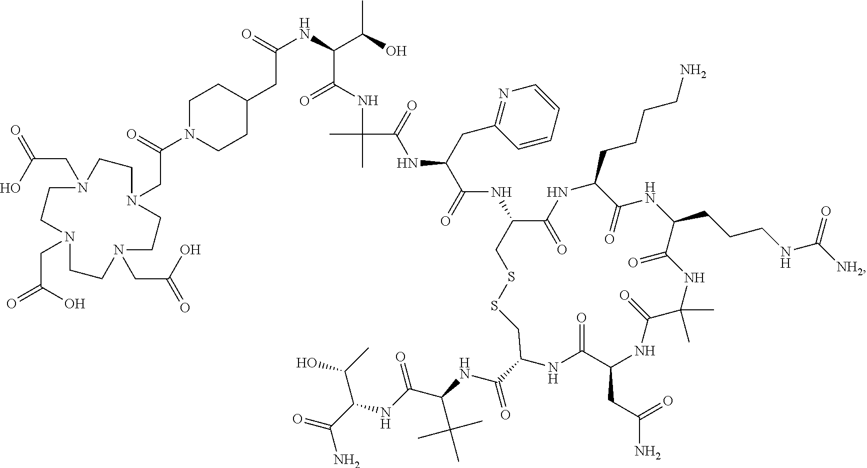 [SMILES: C[C@@H](O)[C@H](NC(=O)[C@@H](NC(=O)[C@@H]1CSSC[C@H](NC(=O)[C@H](Cc2ccccn2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound DOTA-Ahx-Aib-Phe-[Cys-Lys-Arg-Aib-Asn-Cys]-Tle-Thr-NH₂ (PSM-0513) of the following formula

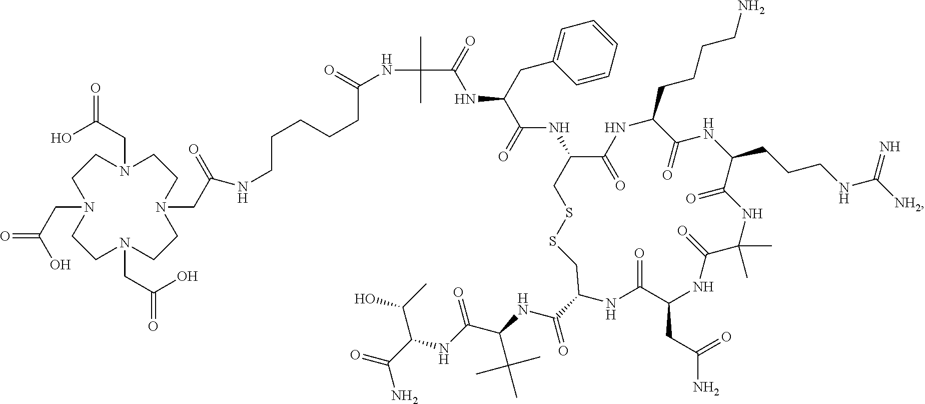 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CCCCCNC(=O)CN2CCN(CC(=O)O)CCN(CC(=O)O)CCN(CC(=O)O)CC2)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Pcf-[Cys-Lys-Arg-Aib-Asn-Cys]-Tle-Bal-NH₂ (PSM-0514) of the following formula

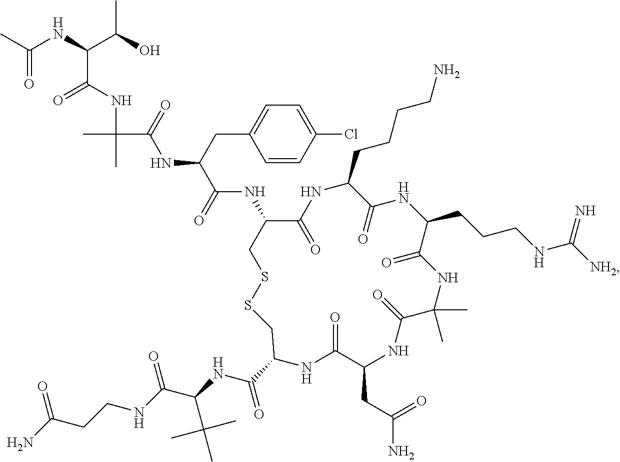 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Macropa-Cmp-Thr-Aib-Pcf-[Cys-Lys(Me)-Arg(Me)-Aib-Asn-Cys]-Tle-Thr-NH₂ (PSM-0515) of the following formula

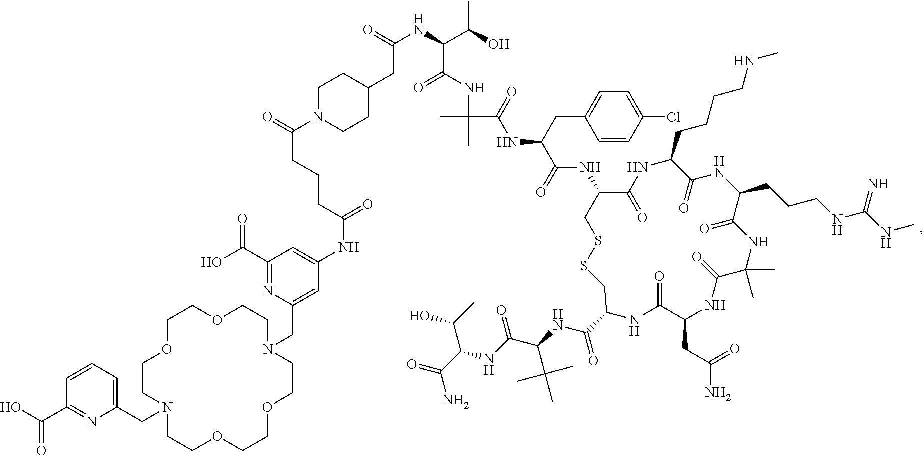 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CCCC(=O)Nc3cc(CN4CCOCCOCCN(Cc5cccc(C(=O)O)n5)CCOCCOCC4)nc(C(=O)O)c3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound Ac-Aib-Pcf-[Cys-Lys-Arg-Aib-As-Cys]-Tle-en-DOTA (PSM-0516) of the following formula

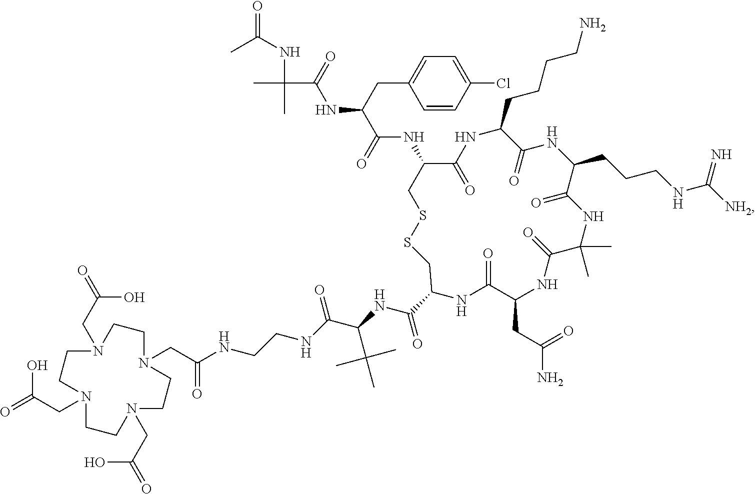 [SMILES: CC(=O)NC(C)(C)C(=O)N[C@@H](Cc1ccc(Cl)cc1)C(=O)N[C@H]1CSSC[C@@H](C(=O)N[C@H](C(=O)NCCNC(=O)CN2CCN(CC(=O)O)CCN(CC(=O)O)CCN(CC(=O)O)CC2)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-2Ni-[Cys-Lys-Arg-Aib-Asn-Cys]-Tle-Thr-Asp-NH₂ (PSM-0517) of the following formula

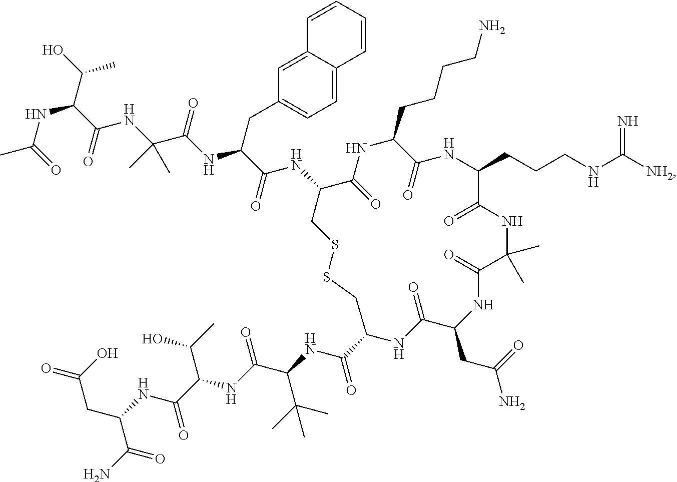 [SMILES: CC(=O)N[C@H](C(=O)NC(C)(C)C(=O)N[C@@H](Cc1ccc2ccccc2c1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound HYDAc-Aib-Phe-[Cys-Lys-Arg-Aib-Asn-Cys]-Tle-Thr-NH₂ (PSM-0518) of the following formula

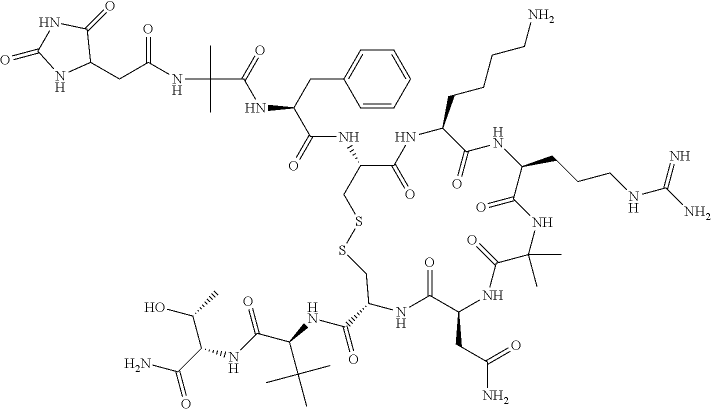 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CC2NC(=O)NC2=O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound InDOTA-Cmp-Thr-Aib-Pcf-[Cys-Lys(Me)-Arg(Me)-Aib-Asn-Cys]-Tle-NH₂ (PSM-0519) of the following formula

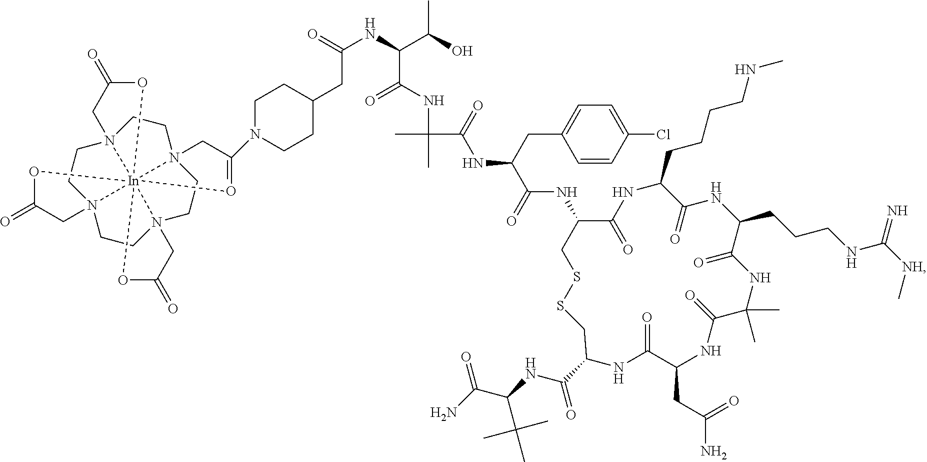

compound Ac-Thr-Aib-Phe-[Cys-Lys-Arg-Lys (InDOTA-020c)-Asn-Cys]-Tle-Thr-NH₂ (PSM-0520) of the following formula

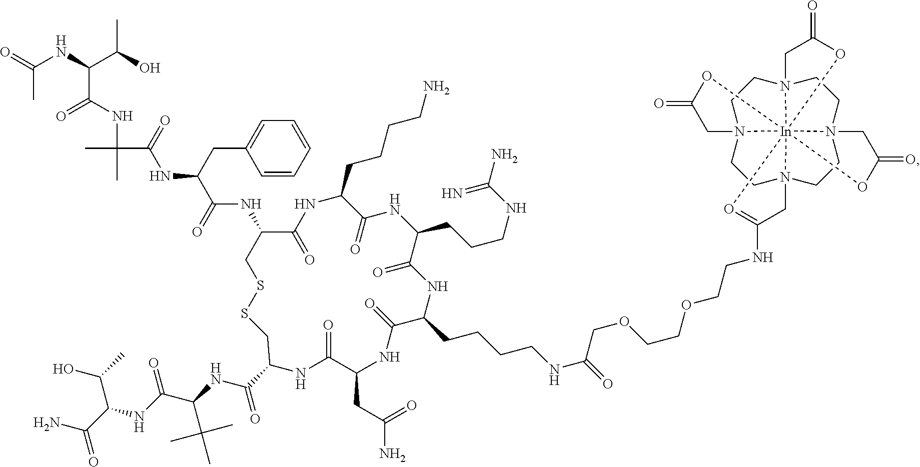

compound DOTA-Ttds-Thr-Aib-Phe-[Cys-Lys-Gln-Aib-Asn-Cys]-Tle-Thr-NH₂ (PSM-0521) of the following formula

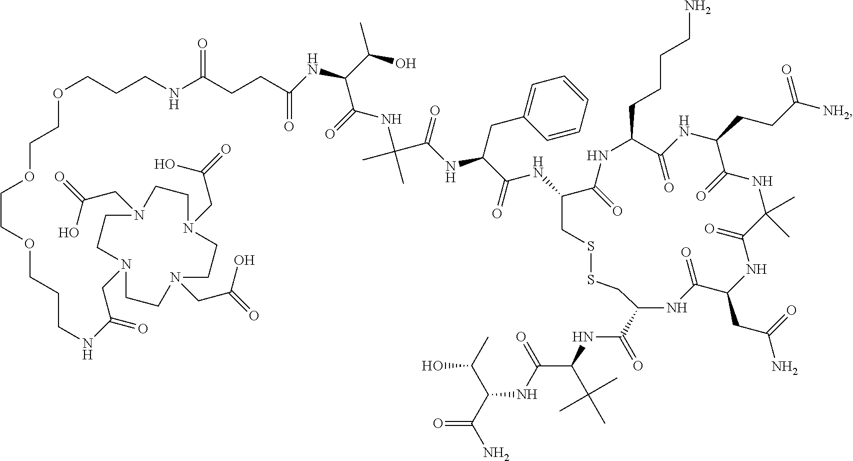 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CCC(=O)NCCCOCCOCCOCCCNC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCC(N)=O)C(=O)NC(C)(C)C(=O)N[C@@H](CC(N)=O)C(=O)N1)C(C)(C)C)C(N)=O]

compound Ac-Thr-Aib-Phe-[Cys-Lys-Arg-Aib-Asn-Cys]-Tle-Thr-Pamb-lys(DOTA)-NH₂ (PSM-0522) of the following formula

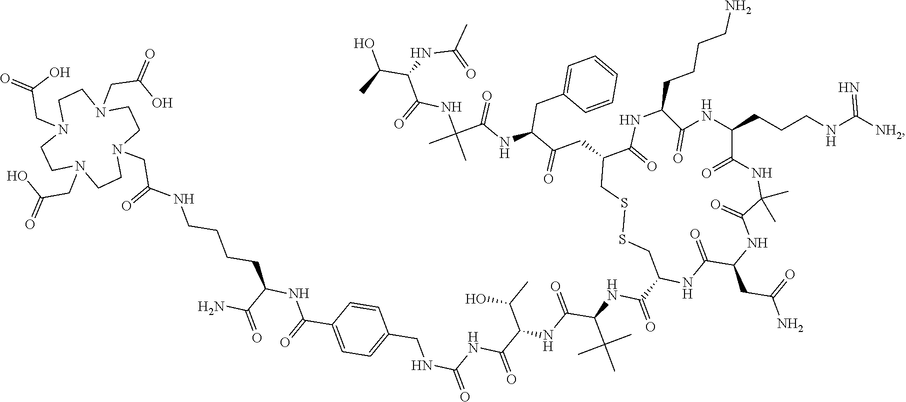 [SMILES: CC(=O)N[C@H](C(=O)NC(C)(C)C(=O)N[C@@H](Cc1ccccc1)C(=O)C[C@H]1CSSC[C@@H](C(=O)N[C@H](C(=O)N[C@H](C(=O)NC(=O)NCc2ccc(C(=O)N[C@H](CCCCNC(=O)CN3CCN(CC(=O)O)CCN(CC(=O)O)CCN(CC(=O)O)CC3)C(N)=O)cc2)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Pcf-[Cys-Lys-Arg-Aib-Asn-Cys]-NH₂ (PSM-0524) of the following formula

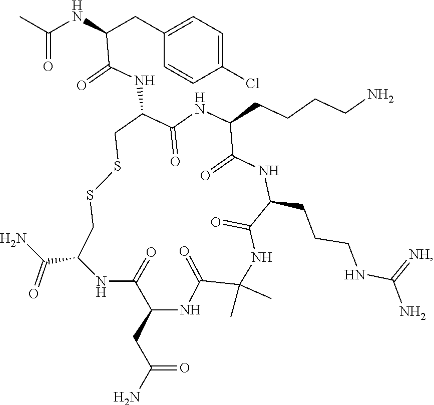 [SMILES: CC(=O)N[C@@H](Cc1ccc(Cl)cc1)C(=O)N[C@H]1CSSC[C@@H](C(N)=O)NC(=O)[C@H](CC(N)=O)NC(=O)C(C)(C)NC(=O)[C@H](CCCNC(=N)N)NC(=O)[C@H](CCCCN)NC1=O]

compound Ac-thr-Als-Phe-[Cys-Lys-Arg-Als-Asn-Cys]-Nle-Thr-Asp-NH₂ (PSM-0525) of the following formula

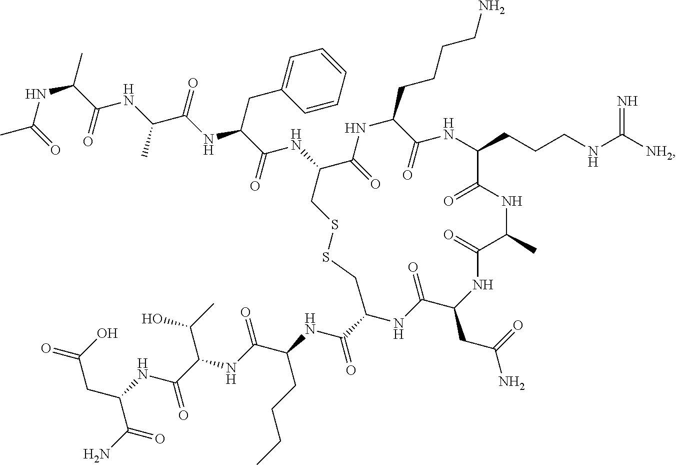 [SMILES: CCCC[C@H](NC(=O)[C@@H]1CSSC[C@H](NC(=O)[C@H](Cc2ccccc2)NC(=O)[C@H](C)NC(=O)[C@H](C)NC(C)=O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound LuDOTA-Cmp-Thr-Aib-Pcf-[Cys-Lys-Arg-Aib-Asn-Cys]-Tle-Thr-NH₂ (PSM-0526) of the following formula

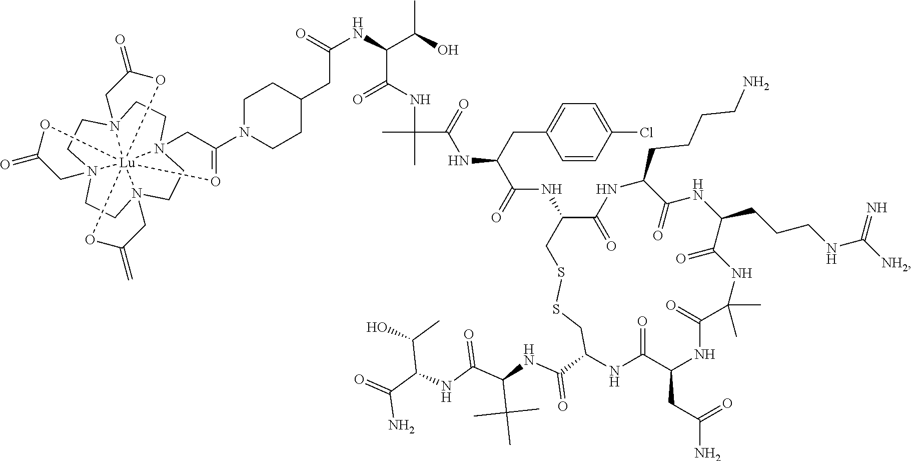

compound InDOTA-Cmp-Thr-Aib-Pcf-[Cys-Lys(Me)-Arg(Me)-Aib-Asn-Cys]-Tle-Thr-NH₂ (PSM-0527) of the following formula

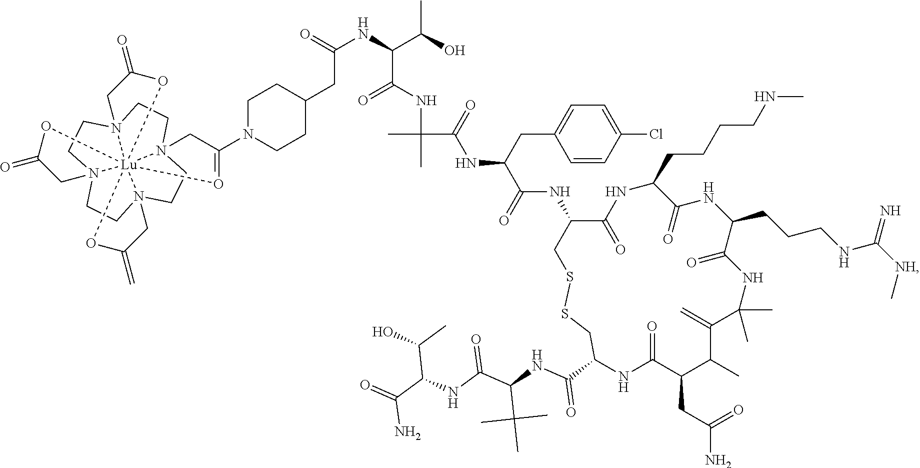

compound Ac-Thr-Aib-Phe-[Cys-Lys-Arg-Aib-Asn-Cys]-Tle-Thr-O2Oc-lys(DOTA)-NH₂ (PSM-0529) of the following formula

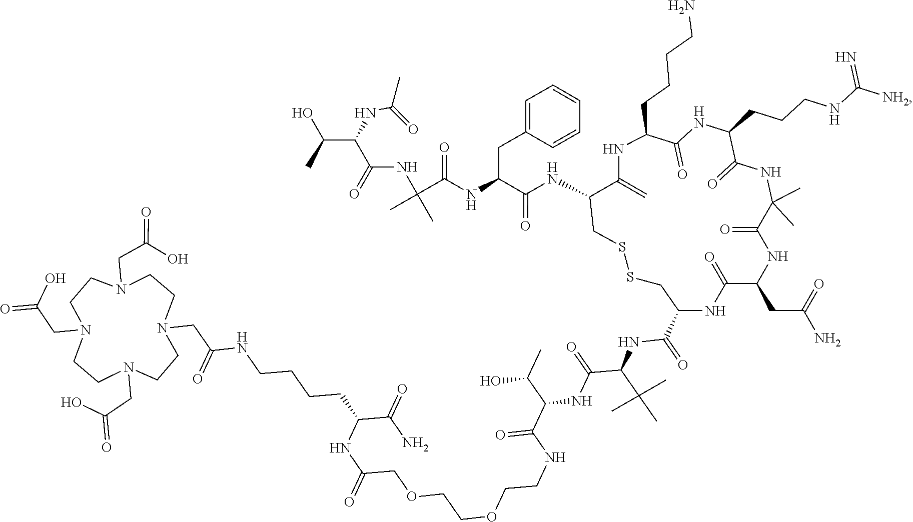 [SMILES: C=C1N[C@@H](CCCCN)C(=O)N[C@@H](CCCNC(=N)N)C(=O)NC(C)(C)C(=O)N[C@@H](CC(N)=O)C(=O)N[C@H](C(=O)N[C@H](C(=O)N[C@H](C(=O)NCCOCCOCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)CSSC[C@@H]1NC(=O)[C@H](Cc1ccccc1)NC(=O)C(C)(C)NC(=O)[C@@H](NC(C)=O)[C@@H](C)O]

compound HPA-Aib-Phe-[Cys-Lys-Arg-Aib-Asn-Cys]-Tle-Thr-NH₂ (PSM-0530) of the following formula

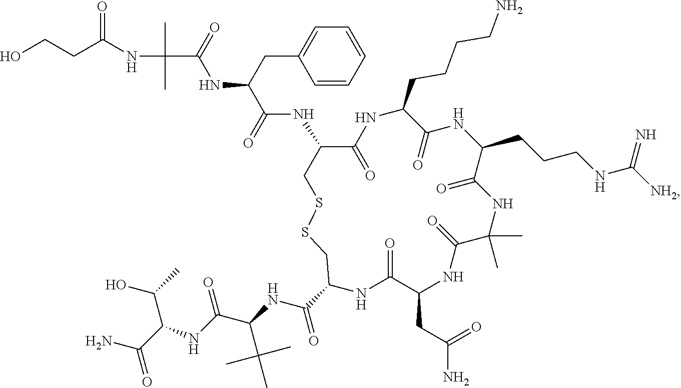 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CCO)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Pcf-[Cys-Lys-Hgn-Aib-Asn-Cys]-Tle-Thr-NH₂ (PSM-0531) of the following formula

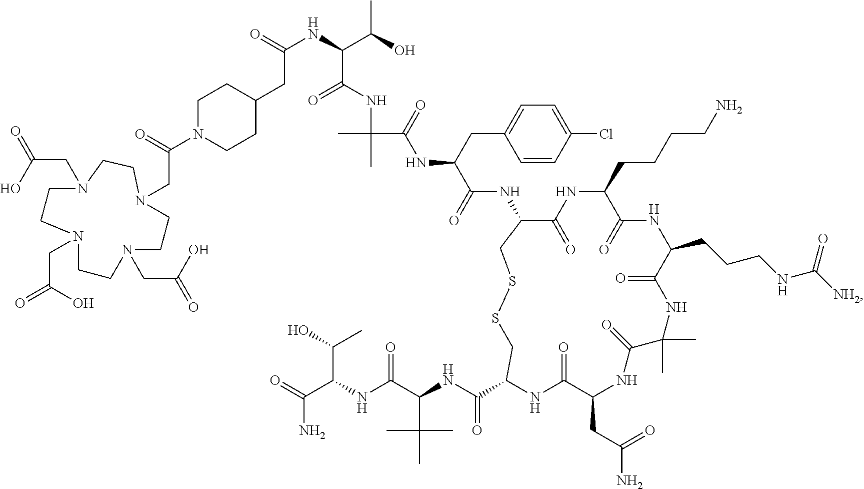 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(N)=O)C(=O)NC(C)(C)C(=O)N[C@@H](CC(N)=O)C(=O)N1)C(C)(C)C)C(N)=O]

compound Ac-Thr-Aib-Phe-[Cys-Lys-Arg-Dtc-Asn-Cys]-Tle-Thr-Asp-NH₂ (PSM-0532) of the following formula

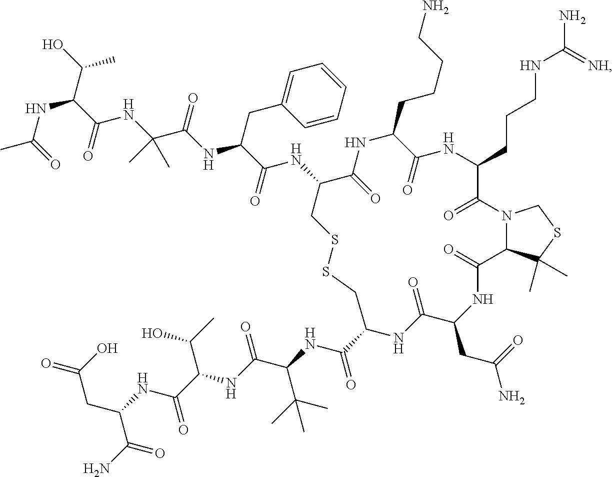 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H]2N(CSC2(C)C)C(=O)[C@H](CCCNC(=N)N)NC(=O)[C@H](CCCCN)NC1=O)[C@@H](C)O]

compound Ac-Thr-Ala-Phe-[Cys-Lys-Arg-Ala-Asn-Cys]-Nle-Thr-Asp-Ttds-lys(DOTA)-NH₂ (PSM-0533) of the following formula

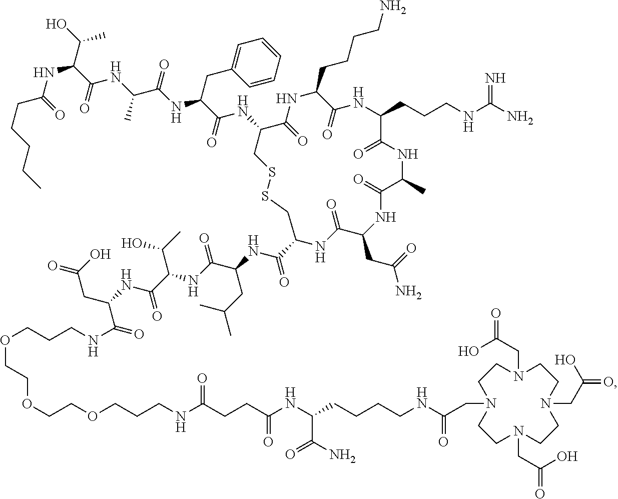 [SMILES: CCCCCC(=O)N[C@H](C(=O)N[C@@H](C)C(=O)N[C@@H](Cc1ccccc1)C(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Thr-Aib-Pcf-[Cys-Lys-Arg-Aib-Asn-Cys]-Tle-NH₂ (PSM-0534) of the following formula

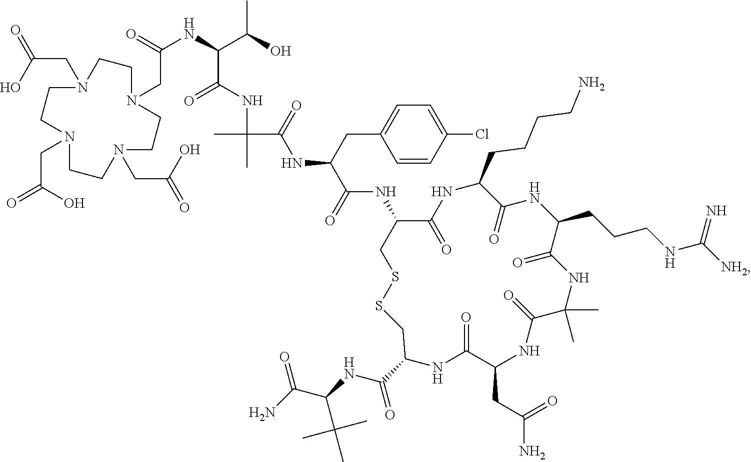 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccc(Cl)cc1)C(=O)N[C@H]1CSSC[C@@H](C(=O)N[C@H](C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-lys-Arg-Ala-Asn-Cys]-Tle-Thr-Asp-NH₂ (PSM-0535) of the following formula

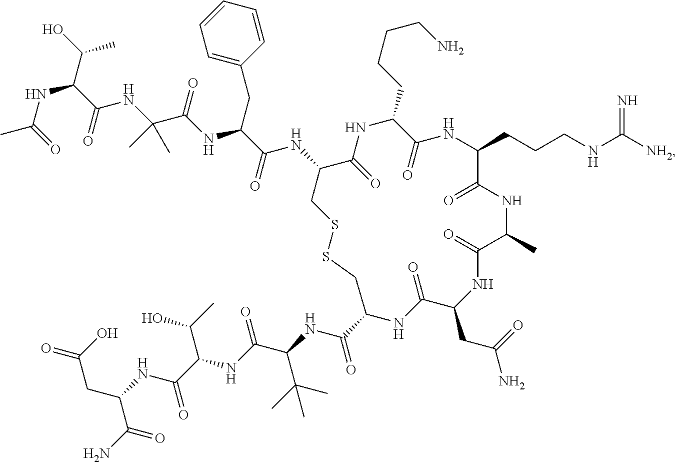 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@@H](CCCCN)NC1=O)[C@@H](C)O]

compound Ac-Thr-Aib-Pcf-[Cys-Lys(Me)-Arg(Me)-Aib-Asn-Cys]-Tle-Thr-Ttds-Ttds-AF488N3K-NH₂ (PSM-0536) of the following formula

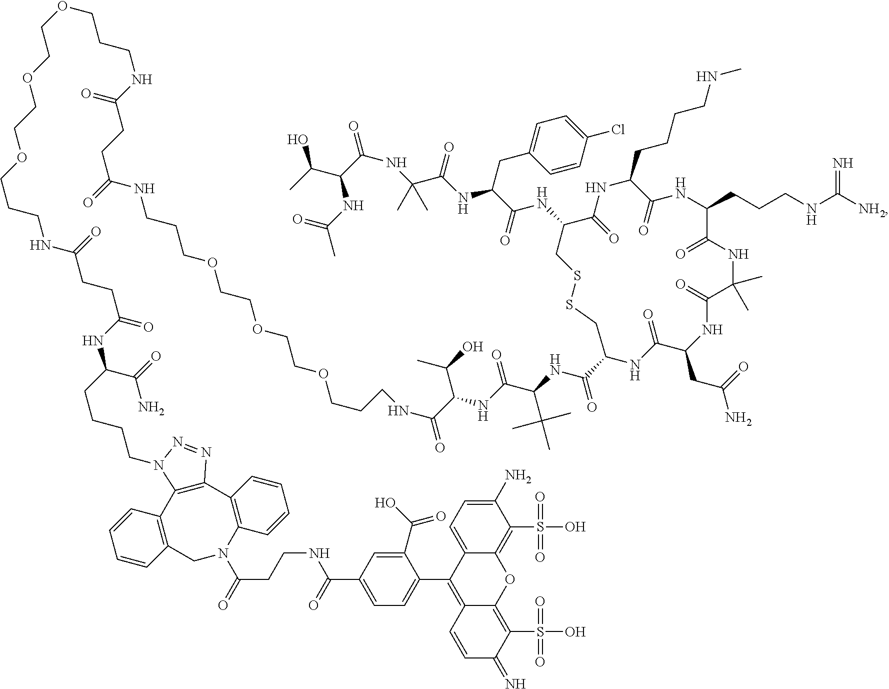 [SMILES: CNCCCC[C@@H]1NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(=O)NCCCOCCOCCOCCCNC(=O)CCC(=O)NCCCOCCOCCOCCCNC(=O)CCC(=O)N[C@H](CCCCn2nnc3c2-c2ccccc2CN(C(=O)CCNC(=O)c2ccc(-c4c5ccc(=N)c(S(=O)(=O)O)c-5oc5c(S(=O)(=O)O)c(N)ccc45)c(C(=O)O)c2)c2ccccc2-3)C(N)=O)[C@@H](C)O)C(C)(C)C)NC(=O)[C@H](CC(N)=O)NC(=O)C(C)(C)NC(=O)[C@H](CCCNC(=N)N)NC1=O]

compound Ac-Thr-ala-Phe-[Cys-Lys-Arg-Ala-Asn-Cys]-Nlc-Thr-Asp-NH₂ (PSM-0537) of the following formula

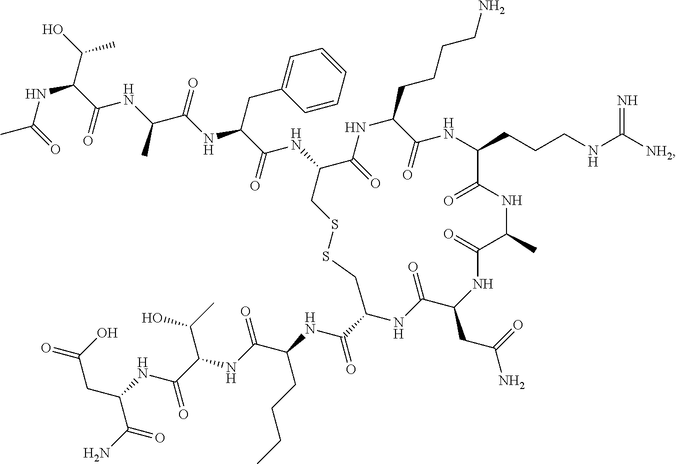 [SMILES: CCCC[C@H](NC(=O)[C@@H]1CSSC[C@H](NC(=O)[C@H](Cc2ccccc2)NC(=O)[C@@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound DOTA-Cmp-Tle-Aib-Pcf-[Cys-Lys (Mc)-Arg (Mc)-Aib-Asn-Cys]-Tle-Thr-NH₂ (PSM-0538) of the following formula

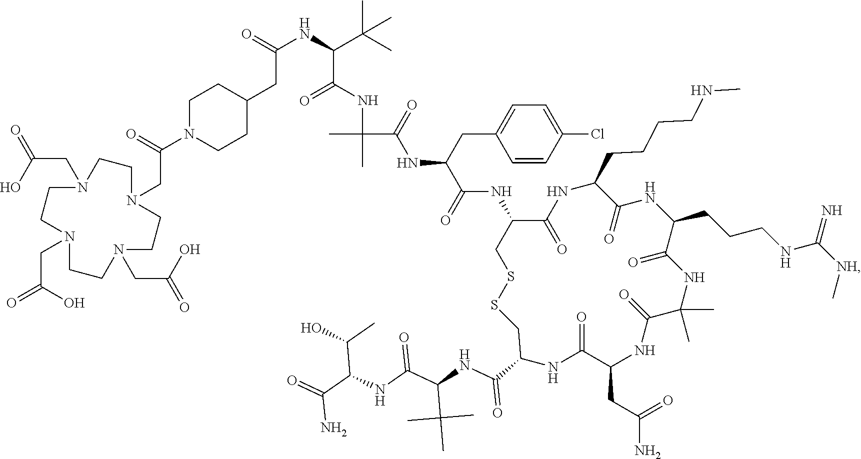 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)C(C)(C)C)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound DOTA-Thr-Aib-Phe-[Cys-Lys-Arg-Aib-Asn-Cys]-Tle-Bal-NH₂ (PSM-0539) of the following formula

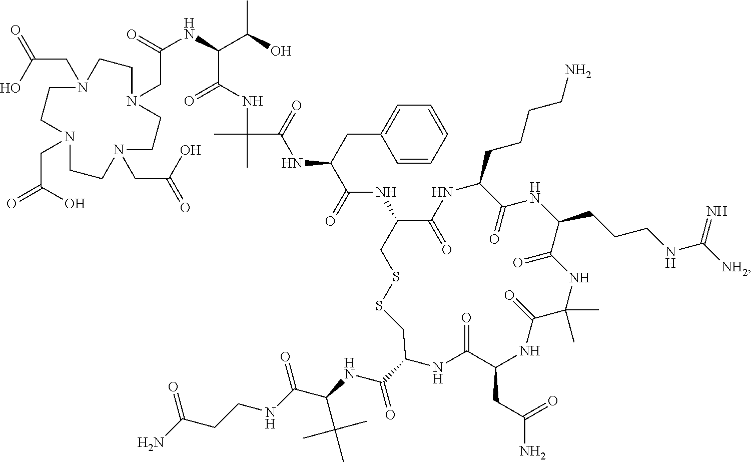 [SMILES: C[C@@H](O)[C@H](NC(=O)CN1CCN(CC(=O)O)CCN(CC(=O)O)CCN(CC(=O)O)CC1)C(=O)NC(C)(C)C(=O)N[C@@H](Cc1ccccc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Lys-Arg-Aib-Asn-Cys]-Tle-Gln-NH₂ (PSM-0540) of the following formula

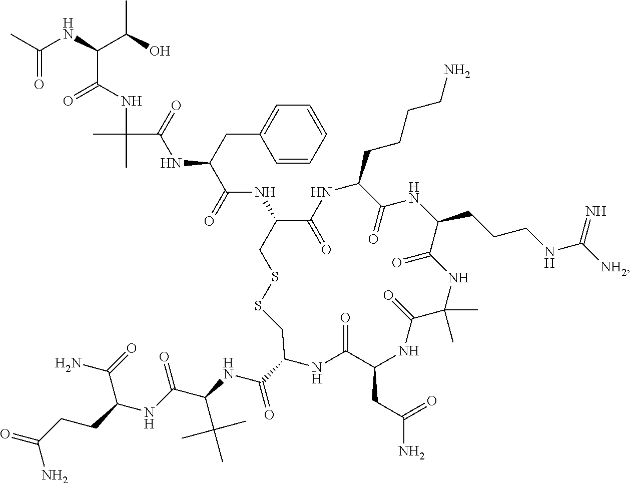 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@@H](CCC(N)=O)C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Phe-Cys]-Tle-NH₂ (PSM-0541) of the following formula

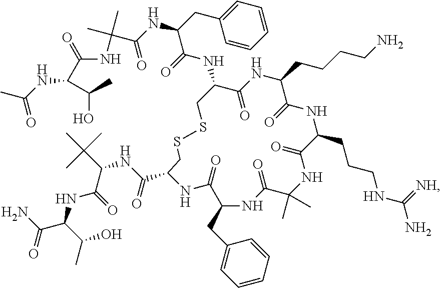 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](Cc2ccccc2)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys(DOTA-Ttds)-Asn-Cys]-Tle-Thr-Asp-NH₂ (PSM-0542) of the following formula

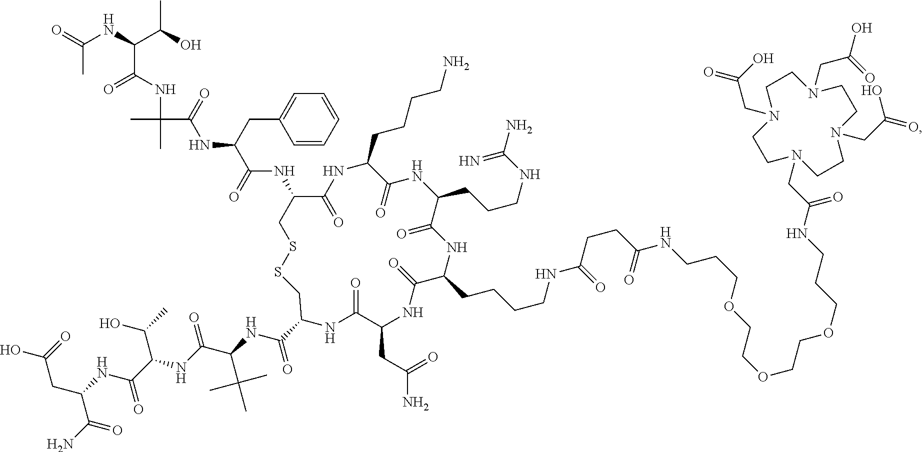 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](CCCCNC(=O)CCC(=O)NCCCOCCOCCOCCCNC(=O)CN2CCN(CC(=O)O)CCN(CC(=O)O)CCN(CC(=O)O)CC2)NC(=O)[C@H](CCCNC(=N)N)NC(=O)[C@H](CCCCN)NC1=O)[C@@H](C)O]

compound DOTA-APAc-Thr-Aib-Phe-[Cys-Lys-Arg-Aib-Asn-Cys]-Tle-Thr-NH₂ (PSM-0543) of the following formula

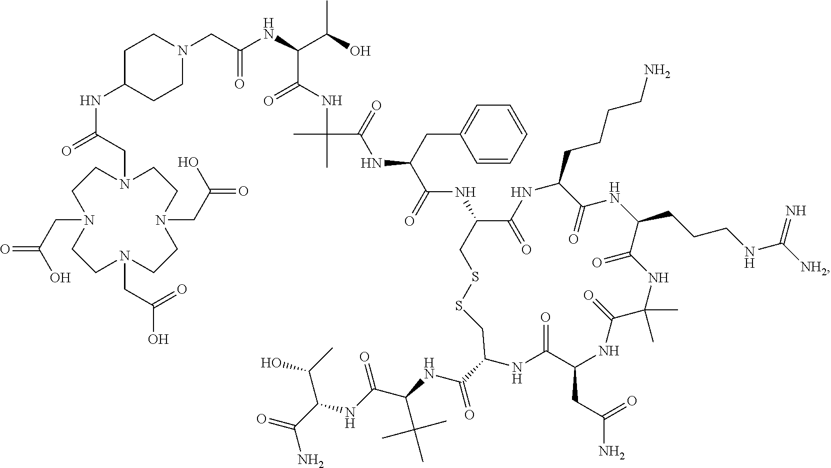 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CN2CCC(NC(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Als-Phe-[Cys-Pro-Arg-Ala-Asn-Cys]-Nle-Thr-Asp-NH₂ (PSM-0544) of the following formula

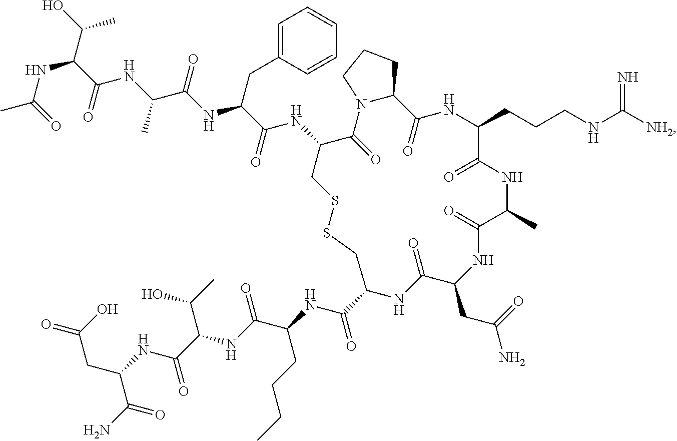 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N2CCC[C@H]2C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Phe-[Cys-Lys-Arg-Aib-Leu-Cys]-Tle-Thr-NH₂ (PSM-0545) of the following formula

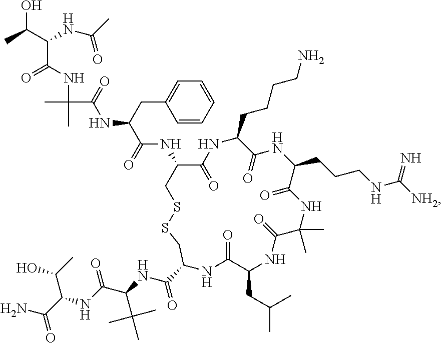 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(C)C)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Urr-Aib-Asn-Cys]-Tle-Thr-NH₂ (PSM-0546) of the following formula

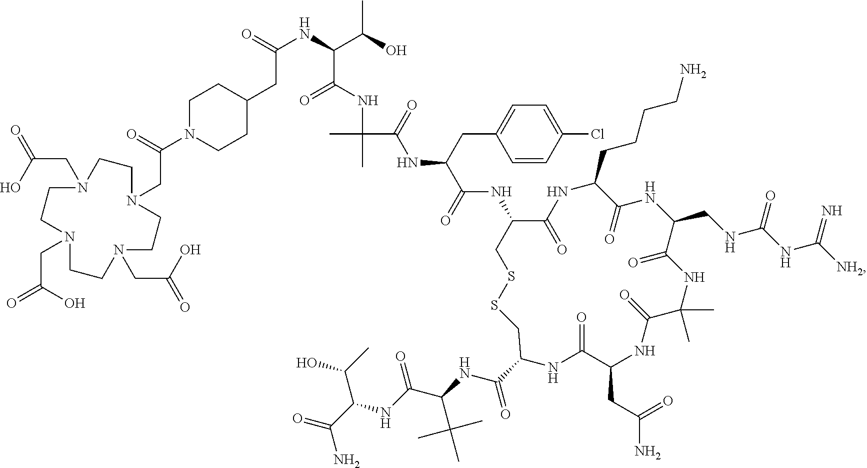 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CNC(=O)NC(=N)N)C(=O)NC(C)(C)C(=O)N[C@@H](CC(N)=O)C(=O)N1)C(C)(C)C)C(N)=O]

compound Ac-Thr-Pro-Phe-[Cys-Lys-Arg-Ala-Asn-Cys]-Nle-Thr-Asp-NH₂ (PSM-0547) of the following formula

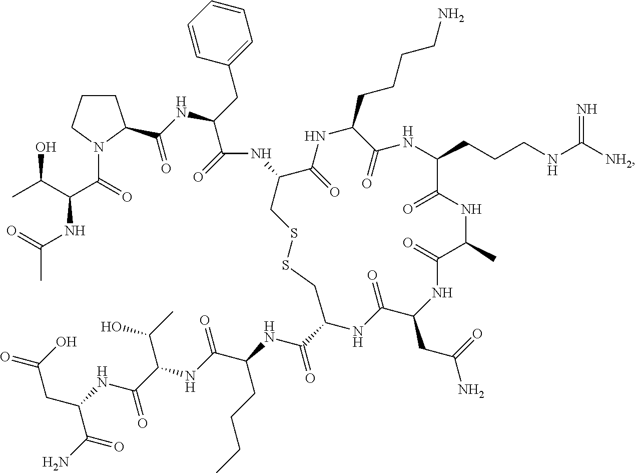 [SMILES: CCCC[C@H](NC(=O)[C@@H]1CSSC[C@H](NC(=O)[C@H](Cc2ccccc2)NC(=O)[C@@H]2CCCN2C(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound DOTA-Cmp-Thr-Aib-Pcf-[Cys-Nmk-Arg-Aib-Asn-Cys]-Tle-Thr-NH₂ (PSM-0548) of the following formula

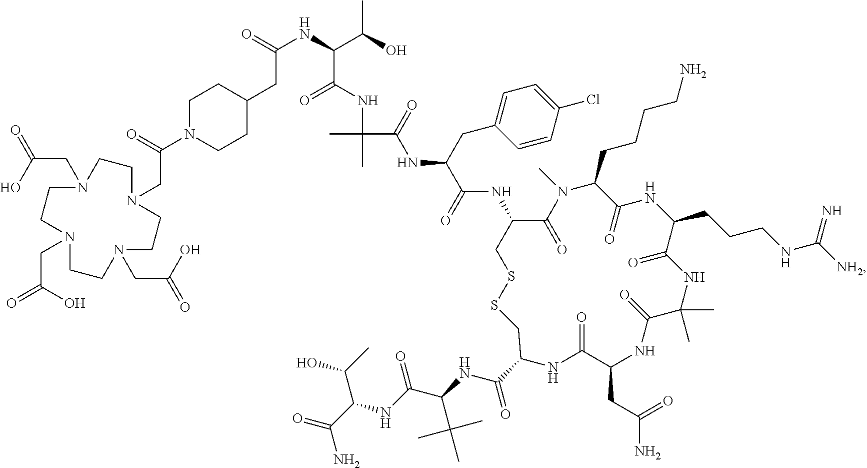 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N(C)[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Cmp-Thr-Aib-Ptf-[Cys-Lys(Me)-Arg(Me)-Aib-Ass-Cys]-Tle-NH₂ (PSM-0549) of the following formula

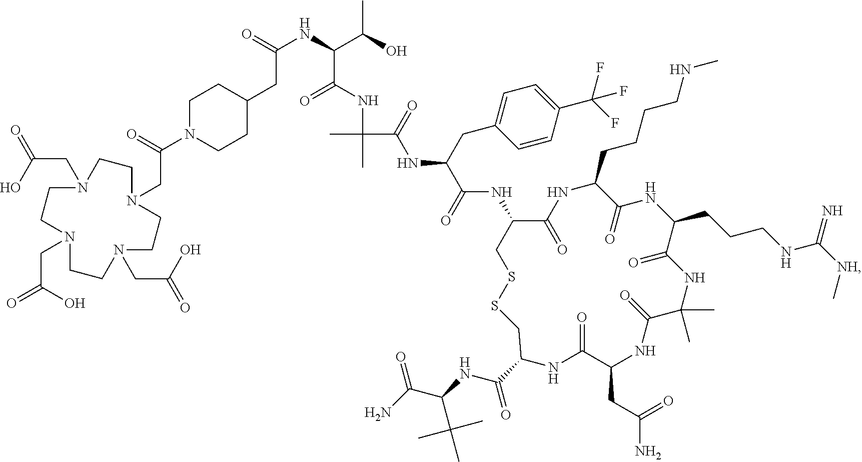 [SMILES: CNCCCC[C@@H]1NC(=O)[C@@H](NC(=O)[C@H](Cc2ccc(C(F)(F)F)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Aib-Ptf-[Cys-Lys-Arg-Aib-Asn-Cys]-Tle-Thr-NH₂ (PSM-0550) of the following formula

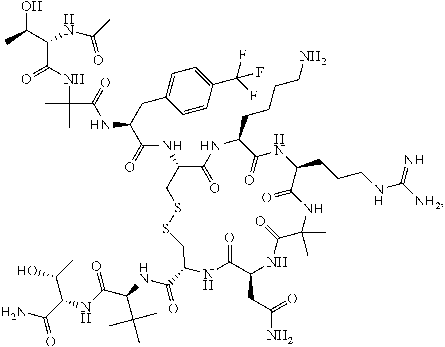 [SMILES: CC(=O)N[C@H](C(=O)NC(C)(C)C(=O)N[C@@H](Cc1ccc(C(F)(F)F)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Mff-[Cys-Lys-Arg-Aib-Asn-Cys]-Tle-Thr-NH₂ (PSM-0551) of the following formula

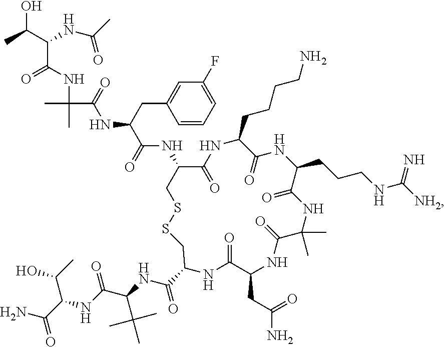 [SMILES: CC(=O)N[C@H](C(=O)NC(C)(C)C(=O)N[C@@H](Cc1cccc(F)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Ala-Asn-Cys]-Tle-Thr-NH₂ (PSM-0552) of the following formula

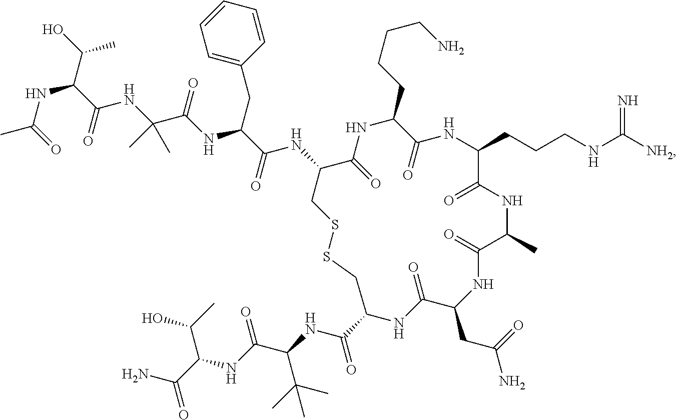 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Mff-[Cys-Lys(Me)-Arg(Me)-Aib-Asn-Cys]-Tle-NH₂ (PSM-0553) of the following formula

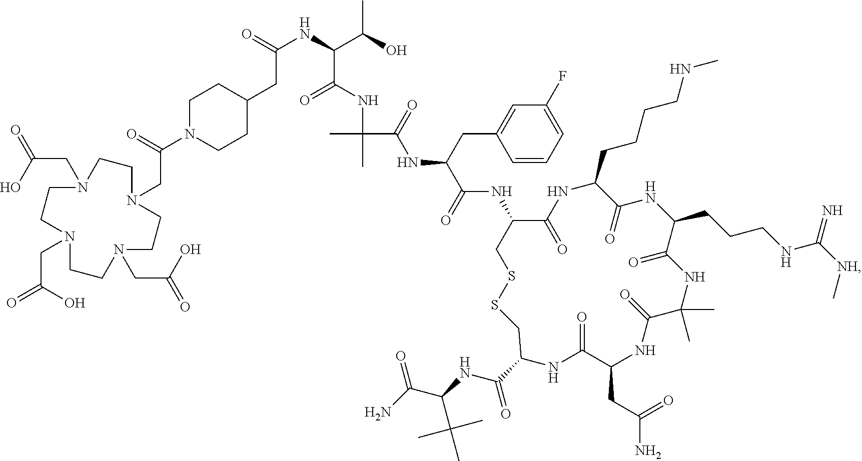 [SMILES: CNCCCC[C@@H]1NC(=O)[C@@H](NC(=O)[C@H](Cc2cccc(F)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(N)=O)C(C)(C)C)NC(=O)[C@H](CC(N)=O)NC(=O)C(C)(C)NC(=O)[C@H](CCCNC(=N)NC)NC1=O]

compound Ac-Thr-Aib-Pcf-[Cys-Lys-Arg-Aib-Asn-Cys]-Tle-Throl-OH (PSM-0554) of the following formula

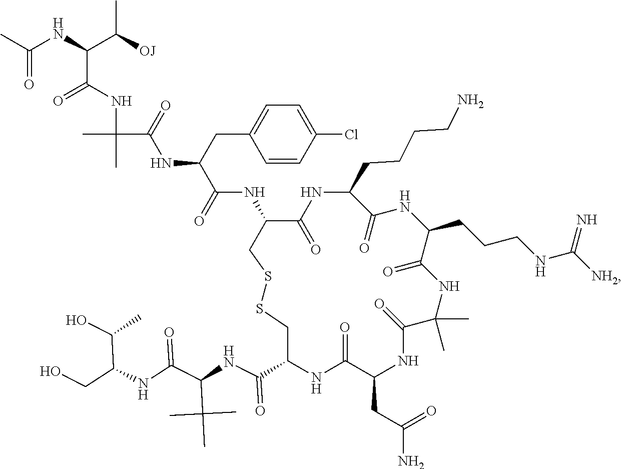

compound Ac-Thr-Aib-Mnf-[Cys-Lys-Arg-Aib-Asn-Cys]-Tle-Thr-NH₂ (PSM-0555) of the following formula

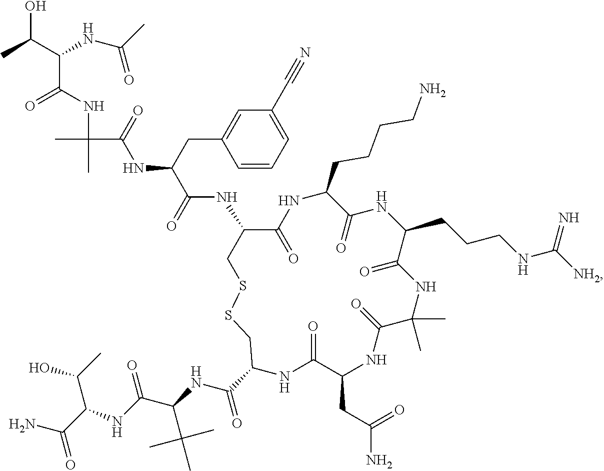 [SMILES: CC(=O)N[C@H](C(=O)NC(C)(C)C(=O)N[C@@H](Cc1cccc(C#N)c1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Asn-Cys]-Tle-Phe-NH₂ (PSM-0556) of the following formula

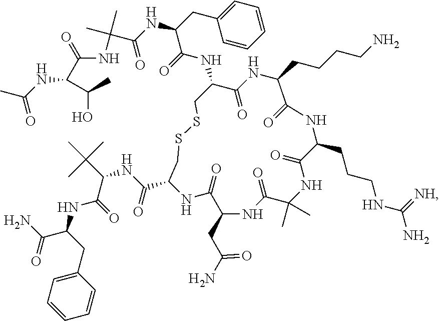 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@@H](Cc2ccccc2)C(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Ocf-[Cys-Lys-Arg-Aib-Asn-Cys]-Tle-Thr-Asp-NH₂ (PSM-0558) of the following formula

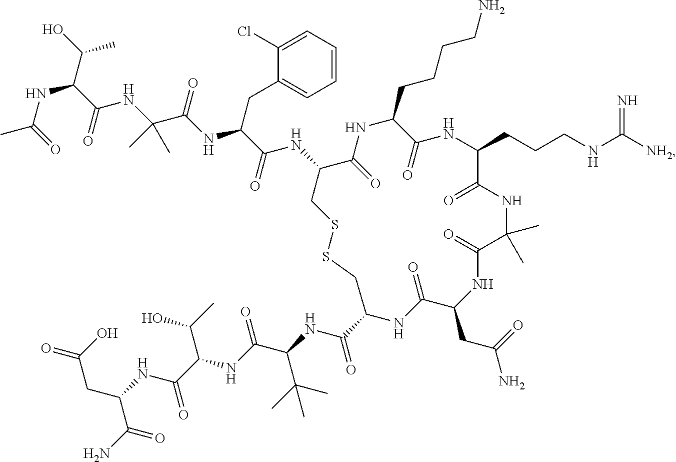 [SMILES: CC(=O)N[C@H](C(=O)NC(C)(C)C(=O)N[C@@H](Cc1ccccc1Cl)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Aib-Pcf-[Cys-Lys-Arg-Aib-Asn-Cys]-Tle-Thr-NH₂ (PSM-0559) of the following formula

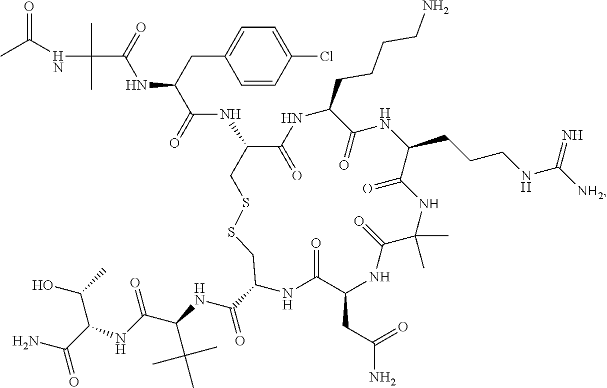 [SMILES: CC(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Ac-Thr-Aib-Phe-[Cys-Lys-Arg-Aib-Asn-Cys]-Npg-NH₂ (PSM-0560) of the following formula

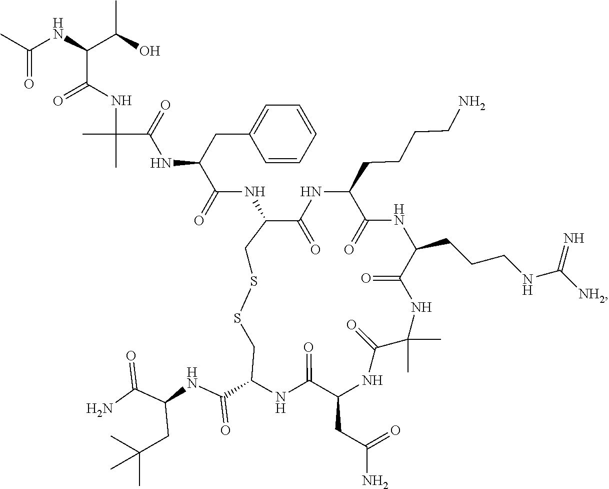 [SMILES: CC(=O)N[C@H](C(=O)NC(C)(C)C(=O)N[C@@H](Cc1ccccc1)C(=O)N[C@H]1CSSC[C@@H](C(=O)N[C@@H](CC(C)(C)C)C(N)=O)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Lys (InDOTA)-Asn-Cys]-Tle-Thr-Asp-NH₂ (PSM-0561) of the following formula

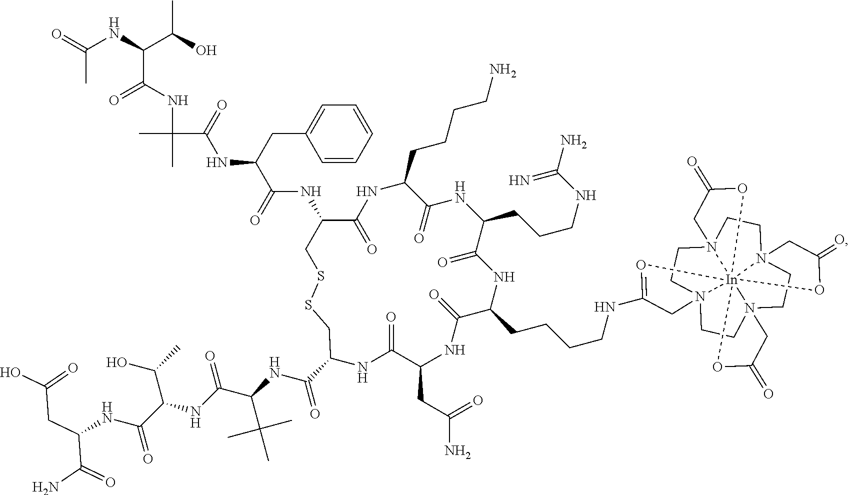

compound Ac-Thr-Als-Phe-[Cys-Lys-Arg-Ala-Asn-Cys]-Nle-Thr-Ala-NH₂ (PSM-0562) of the following formula

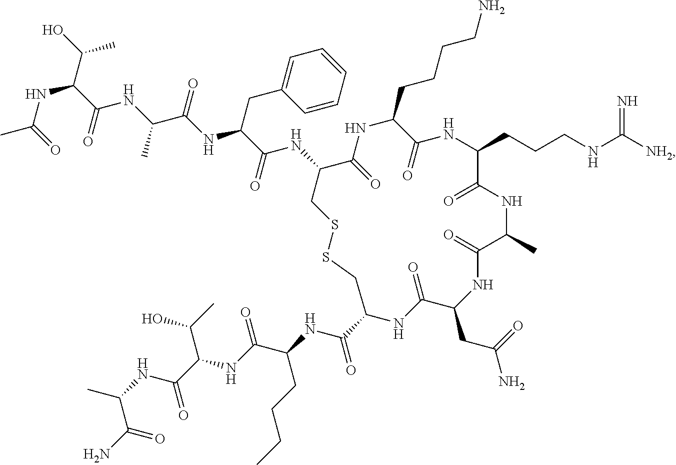 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](C)C(N)=O)[C@@H](C)O]

compound Ac-Thr-Aib-Phe-[Cys-Lys-Arg-Aib-Glu-Cys]-Tle-Thr-NH₂ (PSM-0563) of the following formula

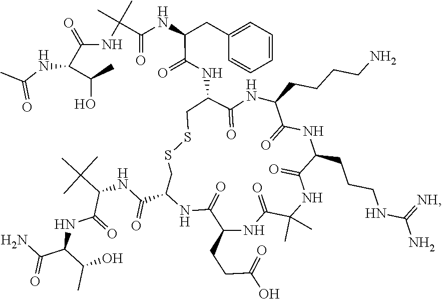 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CCC(=O)O)NC(=O)C(C)(C)NC(=O)[C@H](CCCNC(=N)N)NC(=O)[C@H](CCCCN)NC1=O)[C@@H](C)O]

compound Ac-Thr-Als-Phe-[Cys-Lys-Pro-Ala-Asn-Cys]-Nle-Thr-Asp-NH₂ (PSM-0564) of the following formula

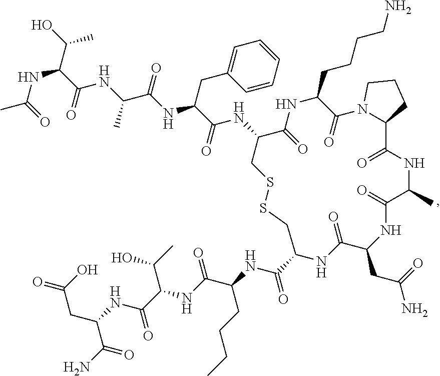 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N2CCC[C@H]2C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Phe-[Cys-Lys-Arg-Ala-Asn-Cys]-Tle-Thr-Asp-NH₂ (PSM-0565) of the following formula

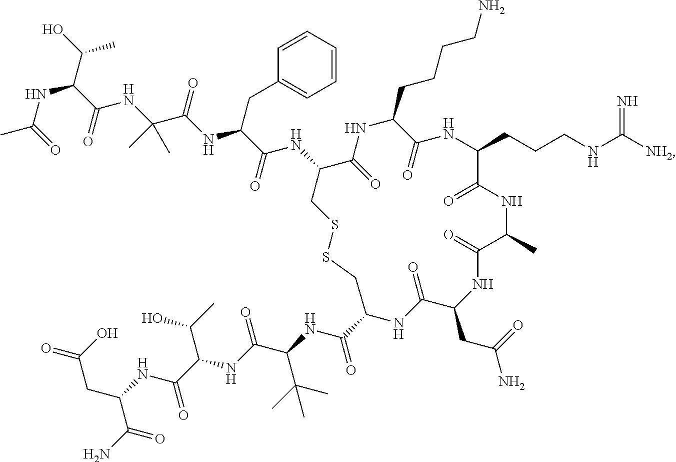 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound DOTA-Cmp-Thr-Aib-Fac-[Cys-Lys-Arg-Aib-Asn-Cys]-Tle-Thr-NH₂ (PSM-0566) of the following formula

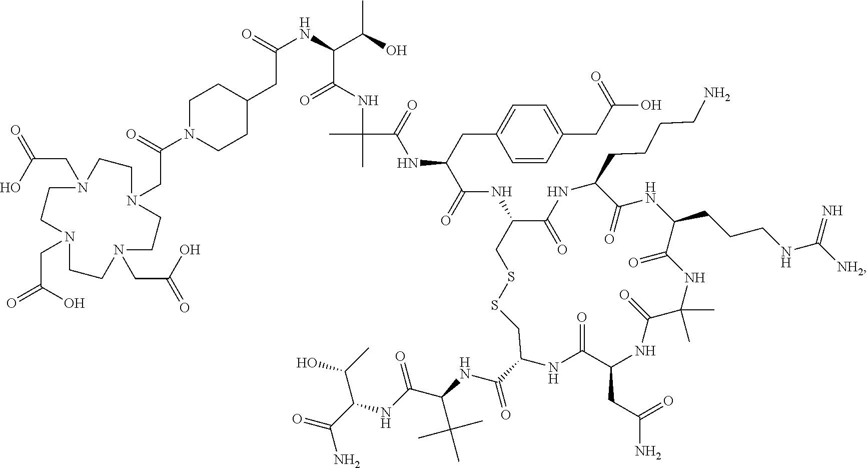 [SMILES: C[C@@H](O)[C@H](NC(=O)[C@@H](NC(=O)[C@@H]1CSSC[C@H](NC(=O)[C@H](Cc2ccc(CC(=O)O)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Phe-[Smc-Lys-Arg-Ala-Asn-Cys]-Tle-Thr-Asp-NH₂ (PSM-0567) (alternative: Ac-Thr-Aib-Phe-[Cys-Lys-Arg-Ala-Asn-Smc]-Tle-Thr-Asp-NH₂) of the following formula

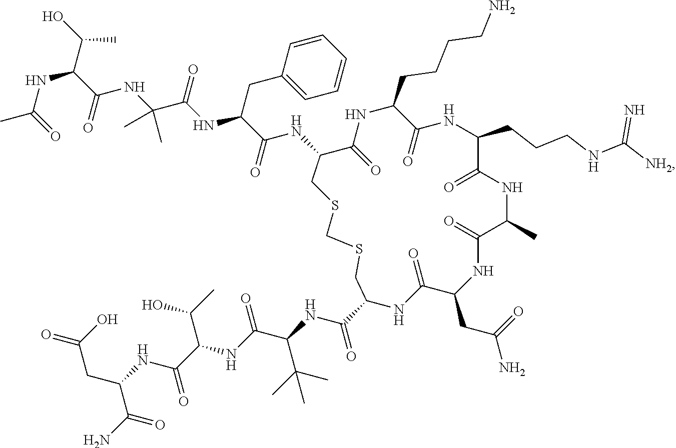 [SMILES: CC(=O)N[C@H](C(=O)NC(C)(C)C(=O)N[C@@H](Cc1ccccc1)C(=O)N[C@H]1CSCSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound Hex-Thr-Ala-Phe-[Cys-Lys-Arg-Ala-Asn-Cys]-Tle-Thr-Asp-Ttds-lys(DOTA)-NH₂ (PSM-0568) of the following formula

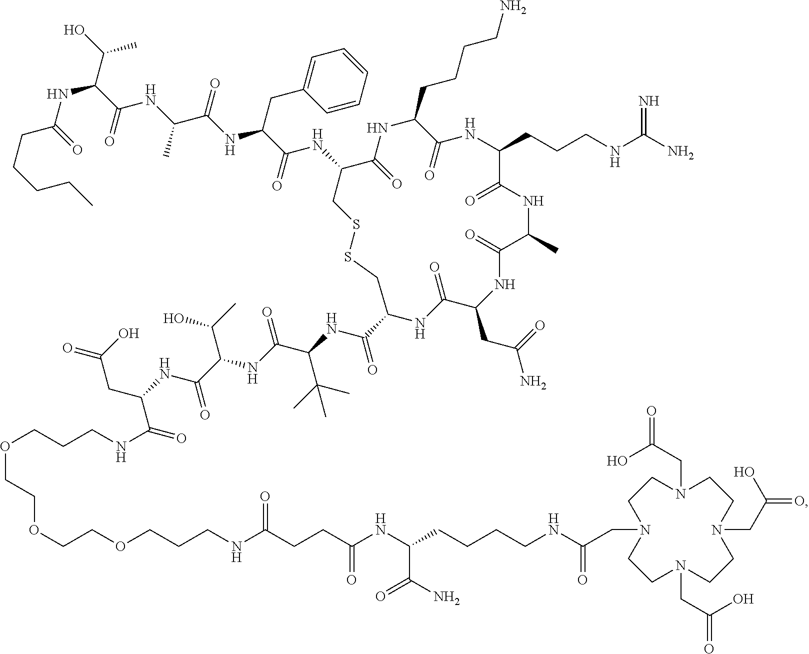 [SMILES: CCCCCC(=O)N[C@H](C(=O)N[C@@H](C)C(=O)N[C@@H](Cc1ccccc1)C(=O)N[C@H]1CSSC[C@@H](C(=O)N[C@H](C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)[C@H](C)NC(=O)[C@H](CCCNC(=N)N)NC(=O)[C@H](CCCCN)NC1=O)[C@@H](C)O]

compound Ac-Thr-Aib-Pcf-[Cys-Lys-Arg(Ac)-Aib-Asn-Cys]-Tle-Thr-NH₂ (PSM-0569) of the following formula

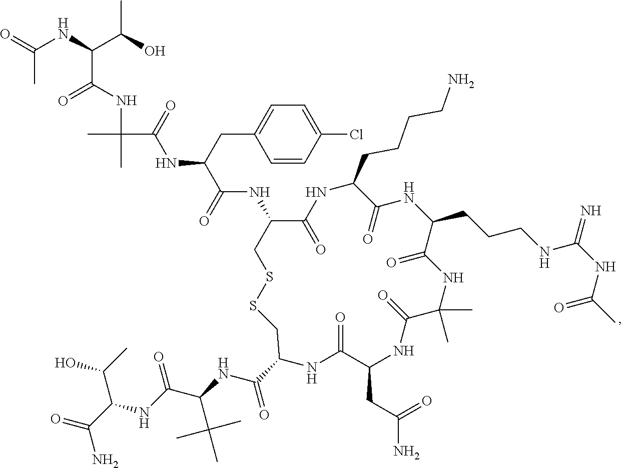 [SMILES: CC(=O)NC(=N)NCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound Ac-Thr-Aib-Pcf-[Cys-Nle-Arg(Ac)-Aib-Asn-Cys]-Tle-Thr-NH₂ (PSM-0570) of the following formula

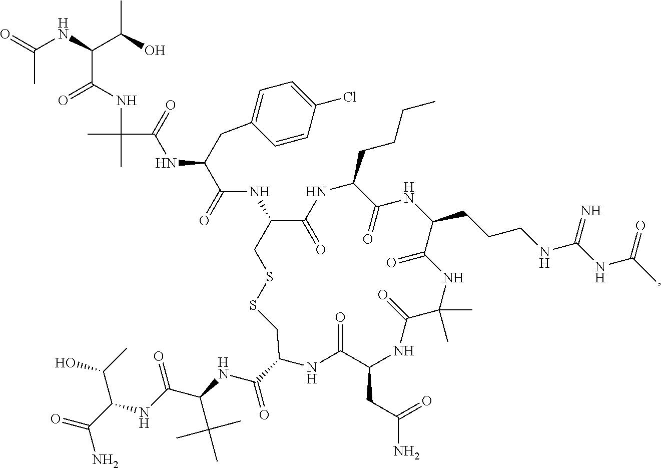 [SMILES: CCCC[C@@H]1NC(=O)[C@@H](NC(=O)[C@H](Cc2ccc(Cl)cc2)NC(=O)C(C)(C)NC(=O)[C@@H](NC(C)=O)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC(C)=O)NC1=O]

compound nBuCAyl-Thr-Aib-Mcf-[Cys-Lys-Opy-Aib-Asn-Cys]-Tle-Thr-Cmp-lys(DOTA)-NH₂ (PSM-0571) of the following formula

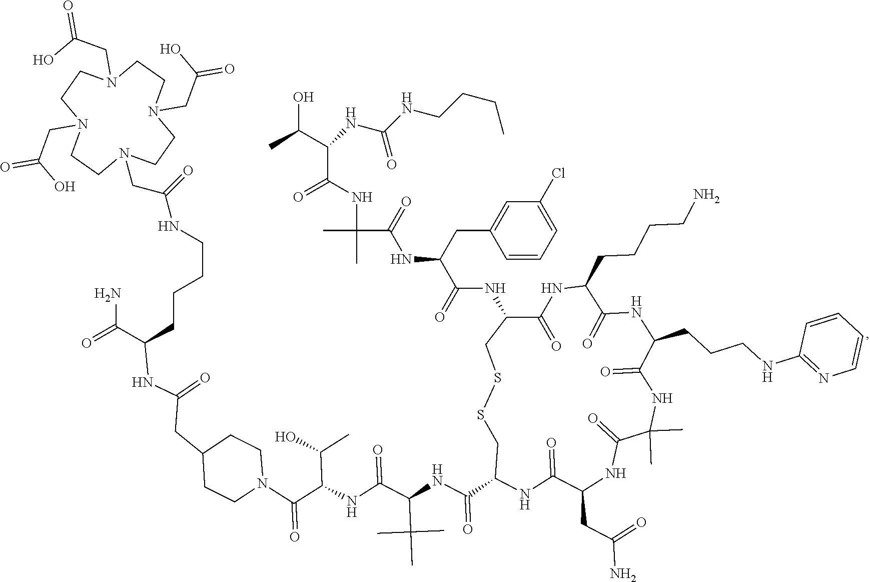 [SMILES: CCCCNC(=O)N[C@H](C(=O)NC(C)(C)C(=O)N[C@@H](Cc1cccc(Cl)c1)C(=O)N[C@H]1CSSC[C@@H](C(=O)N[C@H](C(=O)N[C@H](C(=O)N2CCC(CC(=O)N[C@H](CCCCNC(=O)CN3CCN(CC(=O)O)CCN(CC(=O)O)CCN(CC(=O)O)CC3)C(N)=O)CC2)[C@@H](C)O)C(C)(C)C)NC(=O)[C@H](CC(N)=O)NC(=O)C(C)(C)NC(=O)[C@H](CCCNc2ccccn2)NC(=O)[C@H](CCCCN)NC1=O)[C@@H](C)O]

compound nBuCAyl-Thr-Aib-Phe-[Cys-Lys-Arg-Aib-Asn-Cys]-Tle-Thr-Cmp-lys (LuDOTA)-NH₂ (PSM-0572) of the following formula

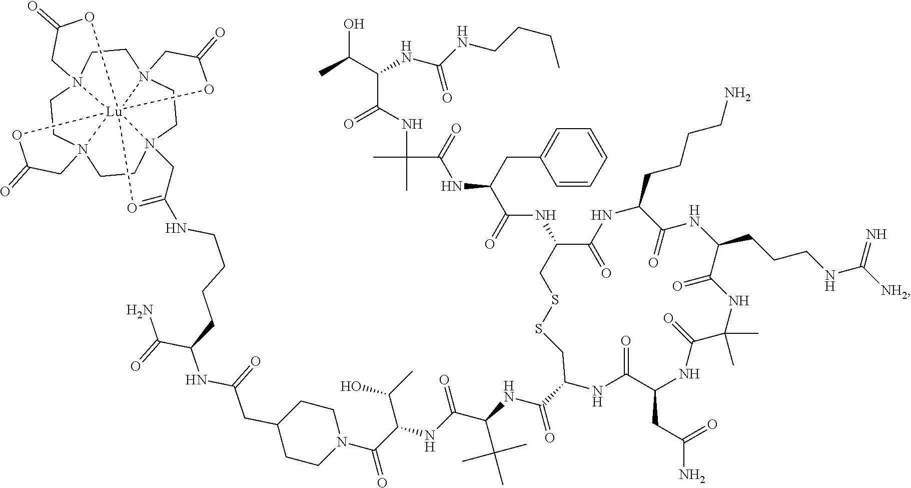

compound DOTA-Thr-Aib-Pcf-[Cys-Lys-Arg-Aib-Asn-Cys]-Tle-Thr-NH₂ (PSM-0573) of the following formula

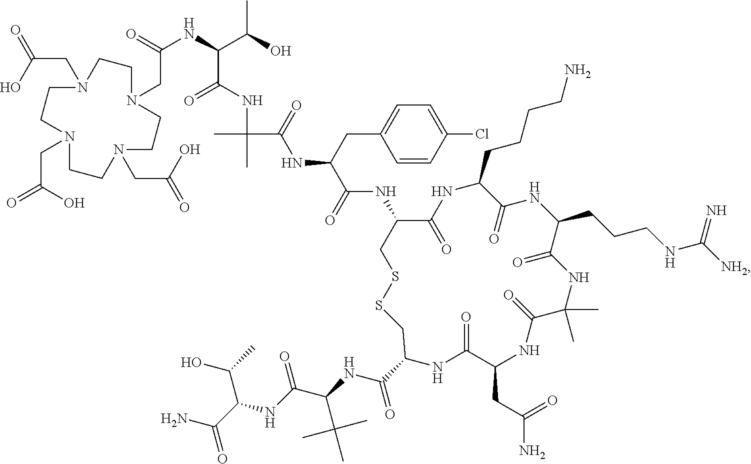 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound DOTA-Pamb-Aib-Phe-[Cys-Lys-Arg-Aib-Asn-Cys]-Tle-Bal-NH₂ (PSM-0574) of the following formula

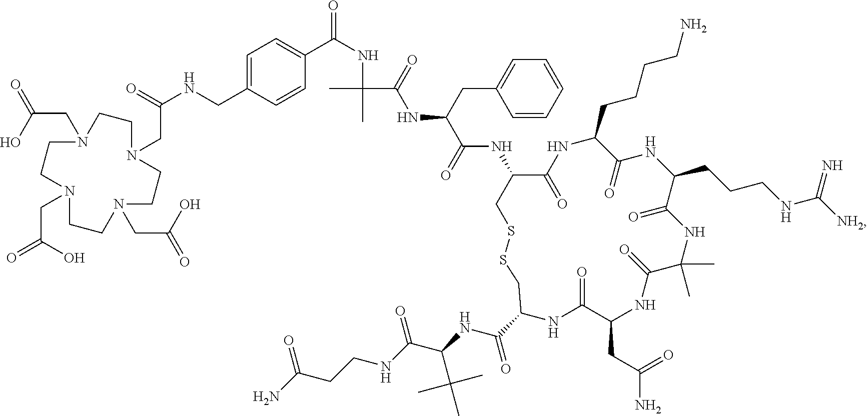 [SMILES: CC(C)(NC(=O)c1ccc(CNC(=O)CN2CCN(CC(=O)O)CCN(CC(=O)O)CCN(CC(=O)O)CC2)cc1)C(=O)N[C@@H](Cc1ccccc1)C(=O)N[C@H]1CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)N)NC(=O)[C@H](CCCCN)NC1=O]

compound Bio-Ttds-Ttds-Thr-Aib-Pcf-[Cys-Lys-Arg-Aib-Asn-Cys]-Tle-Thr-NH₂ (PSM-0575) of the following formula

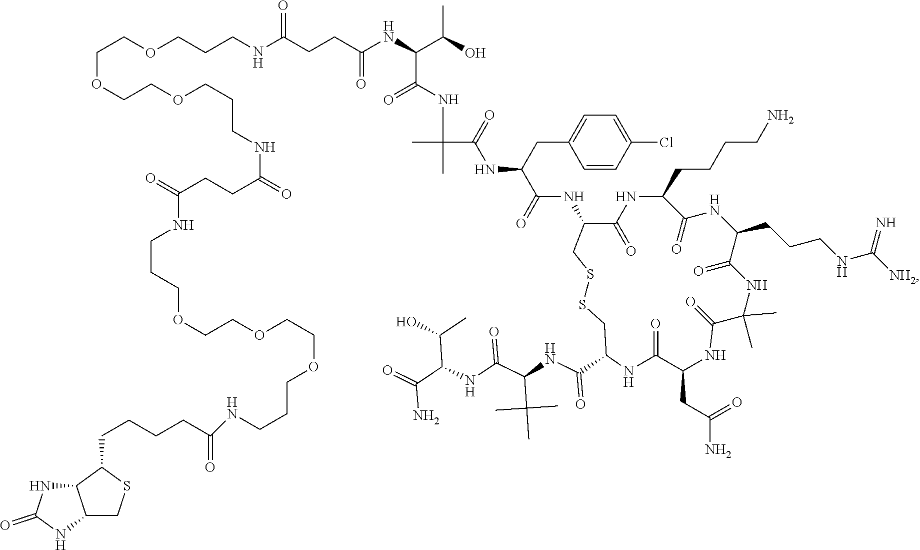 [SMILES: C[C@@H](O)[C@H](NC(=O)[C@@H](NC(=O)[C@@H]1CSSC[C@H](NC(=O)[C@H](Cc2ccc(Cl)cc2)NC(=O)C(C)(C)NC(=O)[C@@H](NC(=O)CCC(=O)NCCCOCCOCCOCCCNC(=O)CCC(=O)NCCCOCCOCCOCCCNC(=O)CCCC[C@@H]2SC[C@@H]3NC(=O)N[C@@H]32)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Hex-Thr-Aib-Phe-[Cys-Lys-Arg-Aib-Asn-Cys]-Tle-Thr-Ttds-lys(DOTA)-NH₂ (PSM-0576) of the following formula

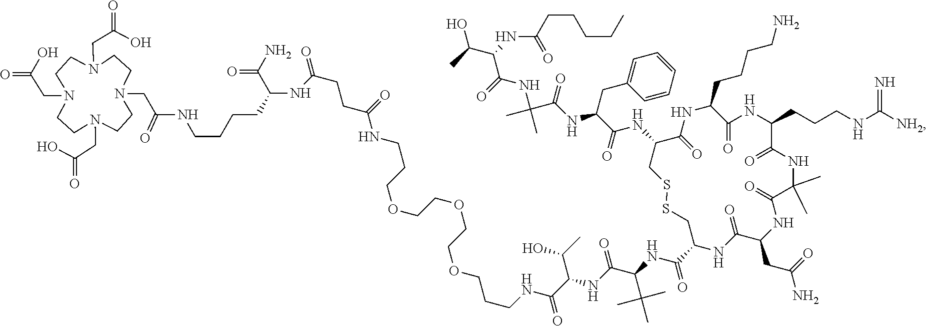 [SMILES: CCCCCC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound HO-Succinyl-Aib-Phe-[Cys-Lys-Arg-Aib-Asn-Cys]-Tle-Thr-NH₂ (PSM-0577) of the following formula

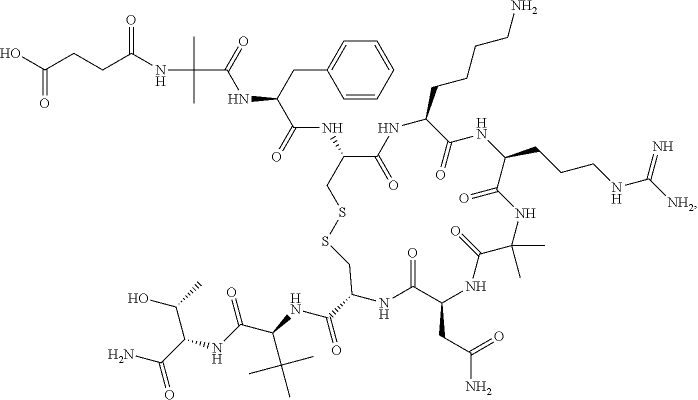 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)CCC(=O)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Ala-Phe-[Cys-Lys-Arg-Aib-Asn-Cys]-Nle-Thr-Asp-NH₂ (PSM-0578) of the following formula

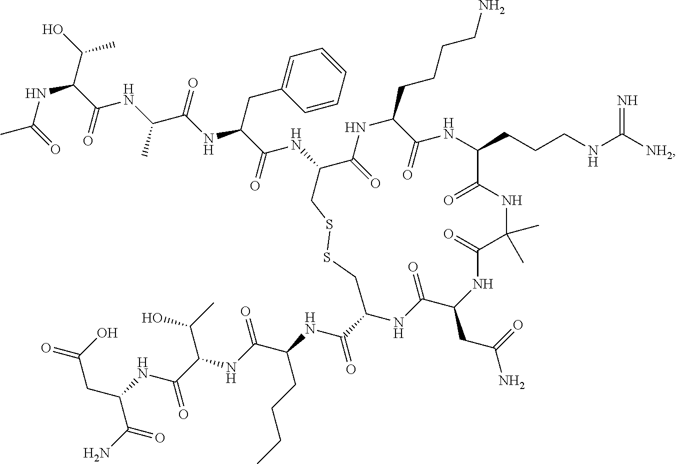 [SMILES: CCCC[C@H](NC(=O)[C@@H]1CSSC[C@H](NC(=O)[C@H](Cc2ccccc2)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(=O)N[C@H](C(=O)N[C@@H](CC(=O)O)C(N)=O)[C@@H](C)O]

compound DOTA-Cmp-Thr-Aib-Pcf-[Cys-Lys-Arg (EtCAyl)-Aib-Asn-Cys]-Tle-Thr-NH₂ (PSM-0579) of the following formula

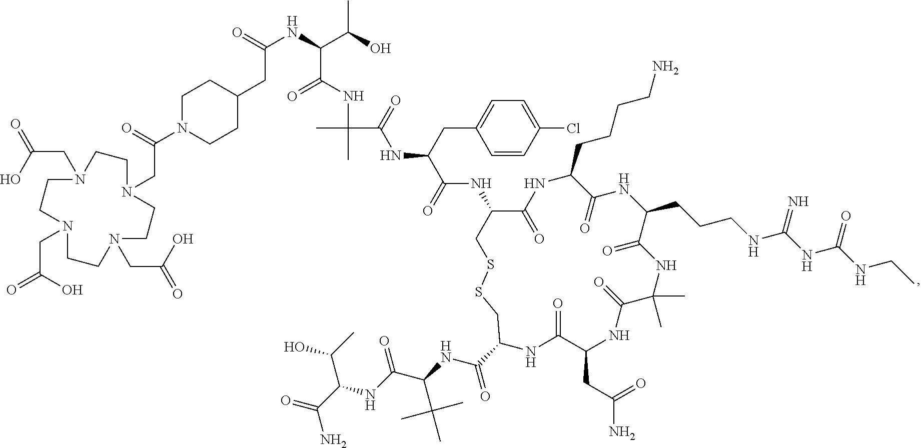 [SMILES: CCNC(=O)NC(=N)NCCC[C@@H]1NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC1=O]

compound DOTA-Thr-Aib-Phe-[Cys-Lys-Arg-Aib-Asn-Cys]-Tle-Thr-NH₂ (PSM-0580) of the following formula

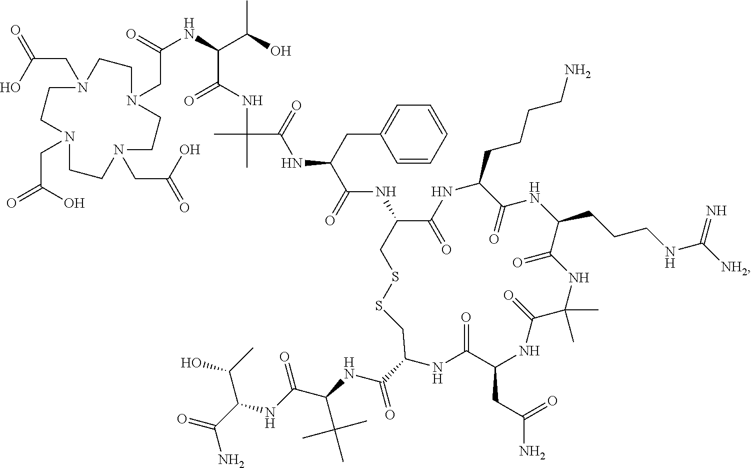 [SMILES: C[C@@H](O)[C@H](NC(=O)[C@@H](NC(=O)[C@@H]1CSSC[C@H](NC(=O)[C@H](Cc2ccccc2)NC(=O)C(C)(C)NC(=O)[C@@H](NC(=O)CN2CCN(CC(=O)O)CCN(CC(=O)O)CCN(CC(=O)O)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Ala-Phe-[Cys-Lys-Ala-Ala-Asn-Cys]-Nle-Thr-Asp-NH₂ (PSM-0581) of the following formula

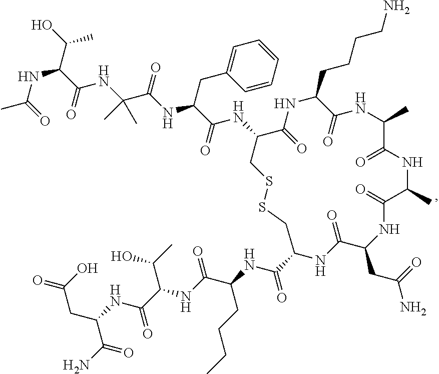 [SMILES: CCCC[C@H](NC(=O)[C@@H]1CSSC[C@H](NC(=O)[C@H](Cc2ccccc2)NC(=O)C(C)(C)NC(=O)[C@@H](NC(C)=O)[C@@H](C)O)C(=O)N[C@@H](CCCCN)C(=O)N[C@@H](C)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Phe-[Cys-Lys-Arg-Lys(DOTA-Ahx)-Asn-Cys]-Tle-Thr-NH₂ (PSM-0582) of the following formula

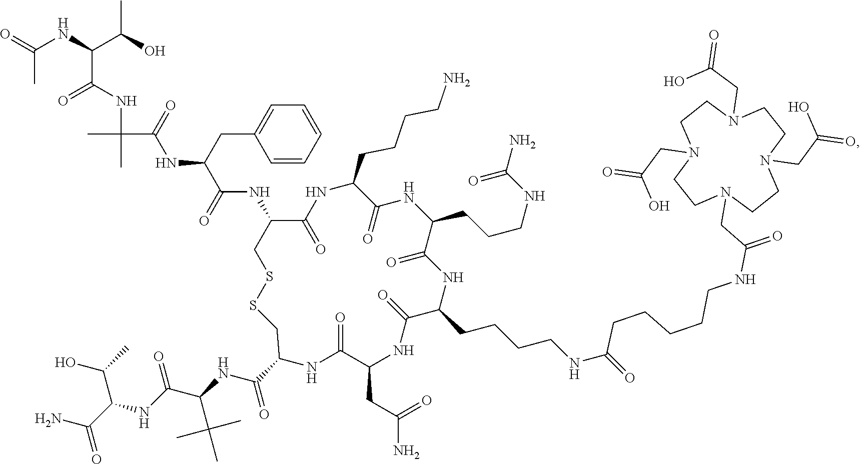 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](CC(N)=O)NC(=O)[C@H](CCCCNC(=O)CCCCCNC(=O)CN2CCN(CC(=O)O)CCN(CC(=O)O)CCN(CC(=O)O)CC2)NC(=O)[C@H](CCCNC(N)=O)NC(=O)[C@H](CCCCN)NC1=O)[C@@H](C)O]

compound DOTA-Cmp-Thr-Aib-Mcf-[Cys-Lys-Arg-Aib-Asn-Cys]-Tle-Thr-NH₂ (PSM-0583) of the following formula

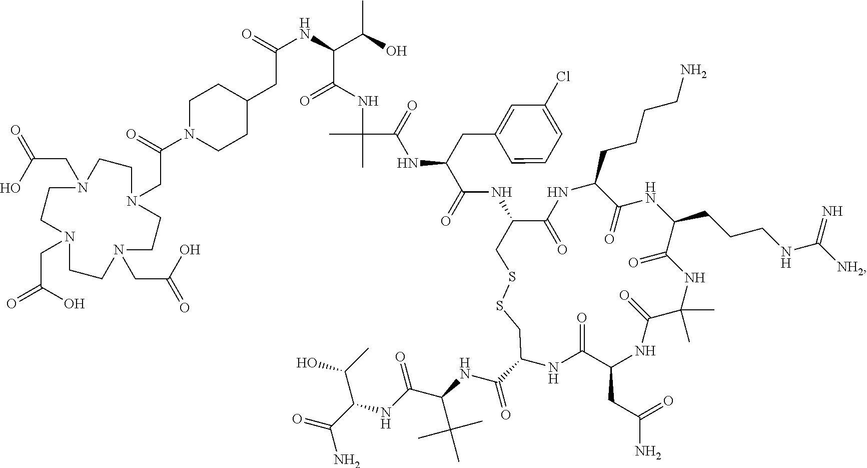 [SMILES: C[C@@H](O)[C@H](NC(=O)[C@@H](NC(=O)[C@@H]1CSSC[C@H](NC(=O)[C@H](Cc2cccc(Cl)c2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)C(=O)N[C@@H](CCCCN)C(=O)N[C@@H](CCCNC(=N)N)C(=O)NC(C)(C)C(=O)N[C@@H](CC(N)=O)C(=O)N1)C(C)(C)C)C(N)=O]

compound Ac-Thr-Aib-Pcf-[Cys-Lys-Cit-Aib-Leu-Cys]-Tle-Thr-NH₂ (PSM-0584) of the following formula

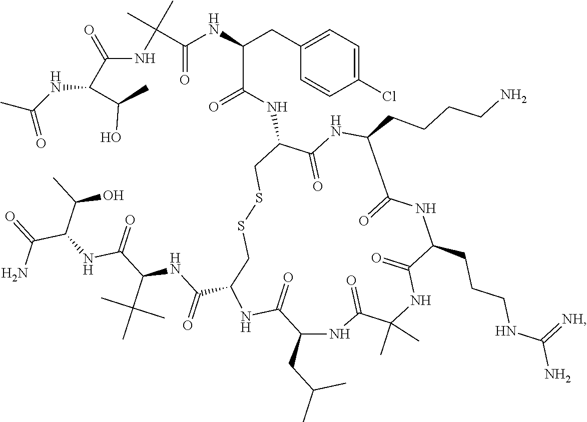 [SMILES: CC(=O)N[C@H](C(=O)NC(C)(C)C(=O)N[C@@H](Cc1ccc(Cl)cc1)C(=O)N[C@H]1CSSC[C@@H](C(=O)N[C@H](C(=O)N[C@H](C(N)=O)[C@@H](C)O)C(C)(C)C)NC(=O)[C@H](CC(C)C)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys-Arg-Ala-Nmn-Cys]-Tle-Thr-NH₂ (PSM-0585) of the following formula

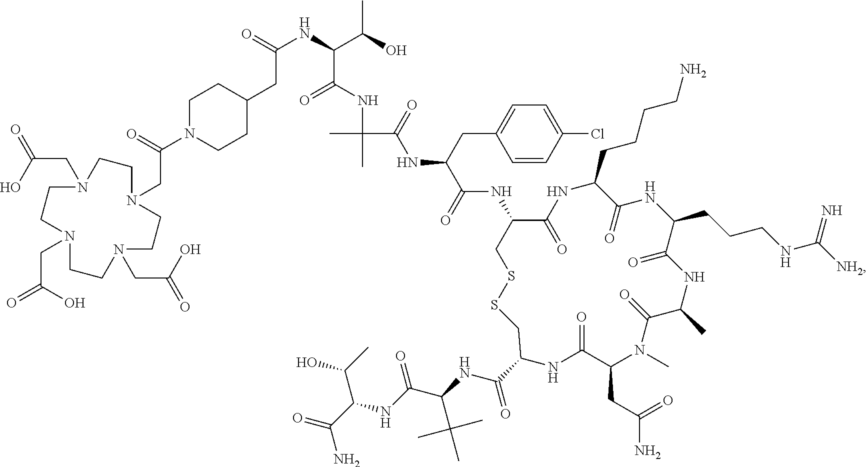 [SMILES: C[C@@H]1NC(=O)[C@H](CCCNC(=N)N)NC(=O)[C@H](CCCCN)NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)N(C)C1=O]

compound Ac-Thr-Ala-Ala-[Cys-Lys-Arg-Ala-Asn-Cys]-Nle-Thr-Asp-NH₂ (PSM-0586) of the following formula

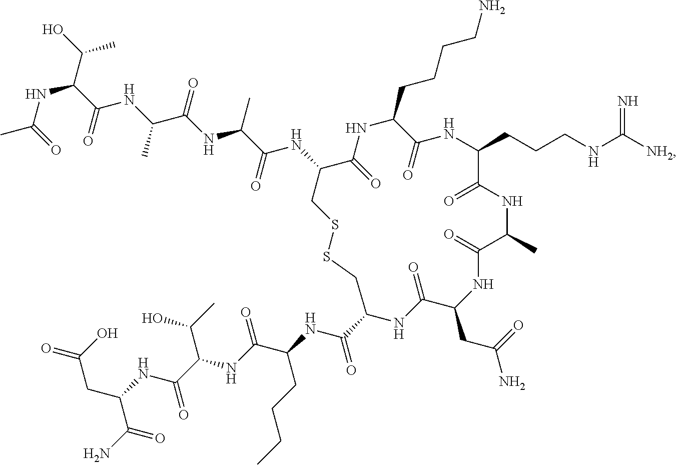 [SMILES: CCCC[C@H](NC(=O)[C@@H]1CSSC[C@H](NC(=O)[C@H](C)NC(=O)[C@H](C)NC(=O)[C@@H](NC(C)=O)[C@@H](C)O)C(=O)N[C@@H](CCCCN)C(=O)N[C@@H](CCCNC(=N)N)C(=O)N[C@@H](C)C(=O)N[C@@H](CC(N)=O)C(=O)N1)C(=O)N[C@H](C(=O)N[C@@H](CC(=O)O)C(N)=O)[C@@H](C)O]

compound Ac-Thr-Aib-Trp-[Cys-Lys-Arg-Aib-Asn-Cys]-Tle-Thr-Asp-NH₂ (PSM-0587) of the following formula

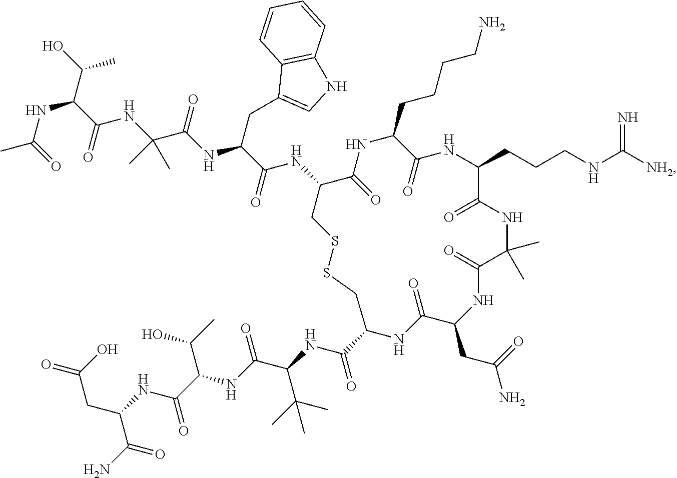 [SMILES: CC(=O)N[C@H](C(=O)NC(C)(C)C(=O)N[C@@H](Cc1c[nH]c2ccccc12)C(=O)N[C@H]1CSSC[C@@H](C(=O)N[C@H](C(=O)N[C@H](C(=O)N[C@@H](CC(=O)O)C(N)=O)[C@@H](C)O)C(C)(C)C)NC(=O)[C@H](CC(N)=O)NC(=O)C(C)(C)NC(=O)[C@H](CCCNC(=N)N)NC(=O)[C@H](CCCCN)NC1=O)[C@@H](C)O]

compound Ac-Thr-Aib-Phe-[Cys-Lys-Arg-Aib-Trp-Cys]-Tle-Thr-NH₂ (PSM-0589) of the following formula

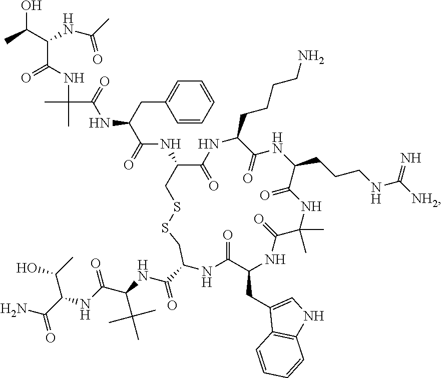 [SMILES: CC(=O)N[C@H](C(=O)NC(C)(C)C(=O)N[C@@H](Cc1ccccc1)C(=O)N[C@H]1CSSC[C@@H](C(=O)N[C@H](C(=O)N[C@H](C(N)=O)[C@@H](C)O)C(C)(C)C)NC(=O)[C@H](Cc2c[nH]c3ccccc23)NC(=O)C(C)(C)NC(=O)[C@H](CCCNC(=N)N)NC(=O)[C@H](CCCCN)NC1=O)[C@@H](C)O]

compound DOTA-Cmp-Thr-Aib-Pcf-[Cys-Lys (Mc)-RMe2a-Aib-Asn-Cys]-Tle-Thr-NH₂ (PSM-0590) of the following formula,

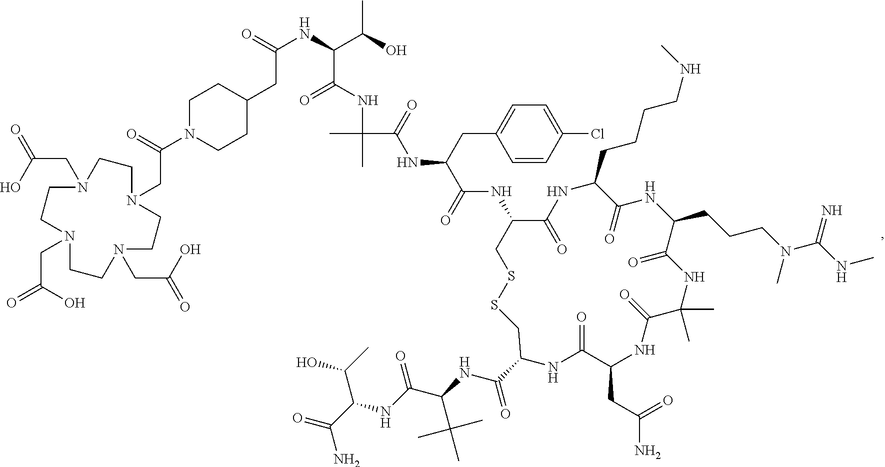 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCN(C)C(=N)NC)NC1=O]

compound Ac-[Cys-Lys-Arg-Ala-Asn-Cys]-Leu-Thr-Asp-Ttds-lys(DOTA)-NH₂ (PSM-0591) of the following formula

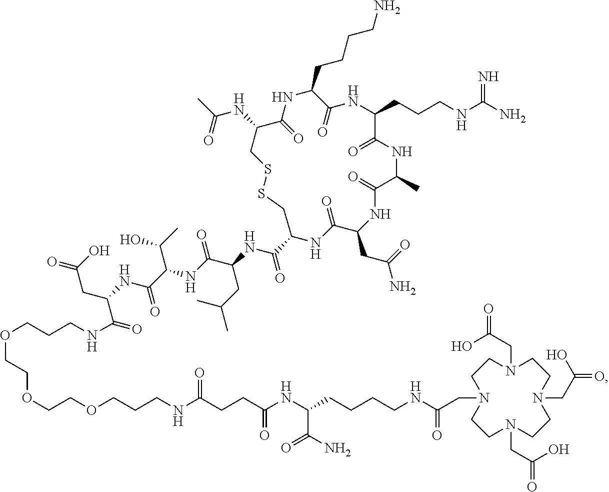 [SMILES: CC(=O)N[C@H]1CSSC[C@@H](C(=O)N[C@@H](CC(C)C)C(=O)N[C@H](C(=O)N[C@@H](CC(=O)O)C(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](CC(N)=O)NC(=O)[C@H](C)NC(=O)[C@H](CCCNC(=N)N)NC(=O)[C@H](CCCCN)NC1=O]

compound DOTA-Cmp-Thr-Aib-Pcf-[Cys-Lys(Me)-RMe1-Aib-Asn-Cys]-Tle-Thr-NH₂ (PSM-0592) of the following formula

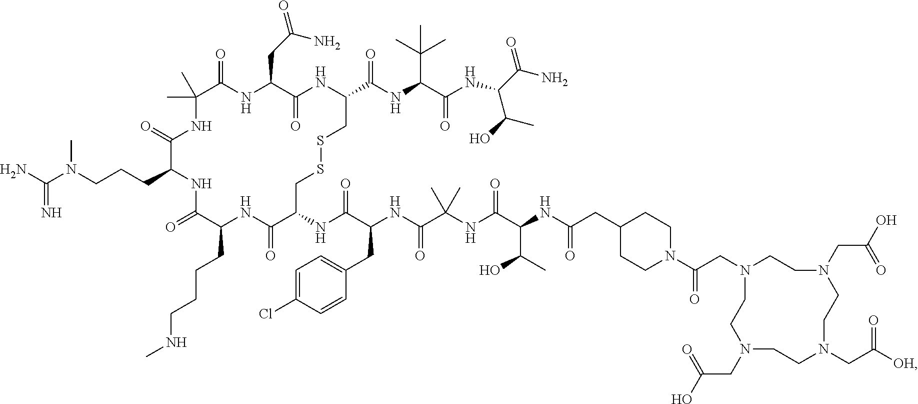 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCN(C)C(=N)N)NC1=O]

compound DOTAM-Cmp-Thr-Aib-Pcf-[Cys-Lys(Me)-Arg(Me)-Aib-Asn-Cys]-Tle-Thr-NH₂ (PSM-0593) of the following formula

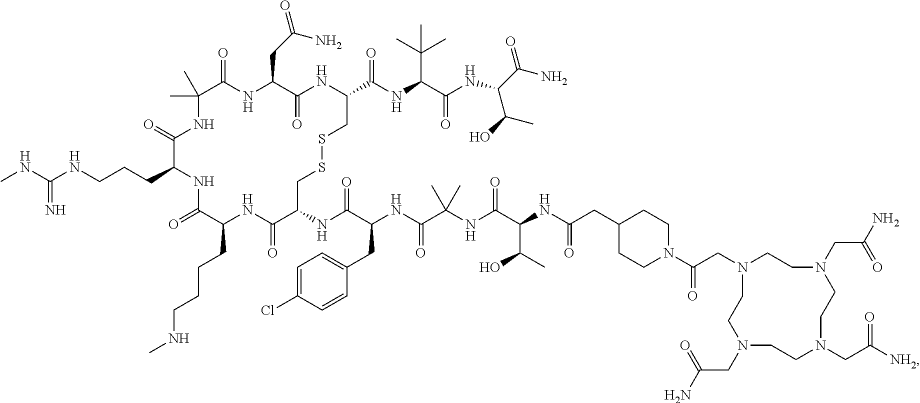 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(N)=O)CCN(CC(N)=O)CCN(CC(N)=O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound DOTA-Cmp-Thr-Aib-Pcf-[Cys-Lys(Me)-Arg(Me)-Aib-Asn-Cys]-OH (PSM-0594) of the following formula

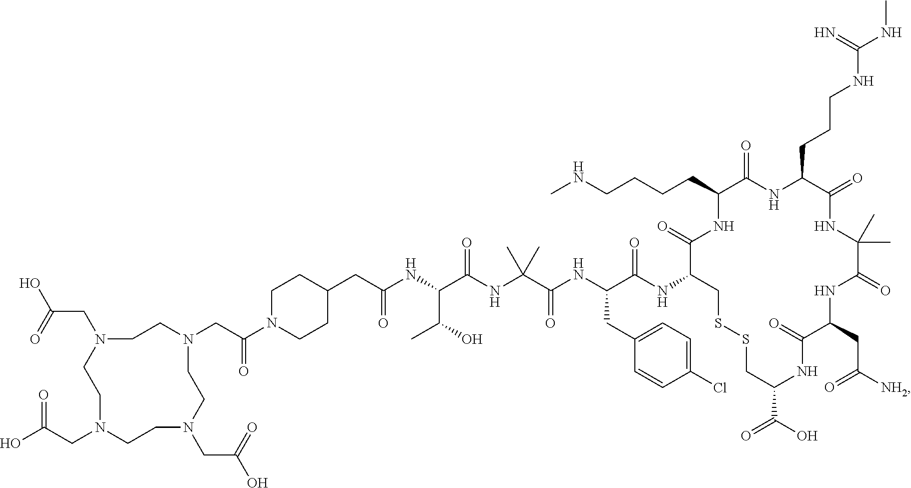 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)O)NC(=O)[C@H](CC(N)=O)NC(=O)C(C)(C)NC(=O)[C@H](CCCNC(=N)NC)NC1=O]

compound BiDOTA-Cmp-Thr-Aib-Pcf-[Cys-Lys(Me)-Arg(Me)-Aib-Asn-Cys]-Tle-Thr-NH₂ (PSM-0595) of the following formula

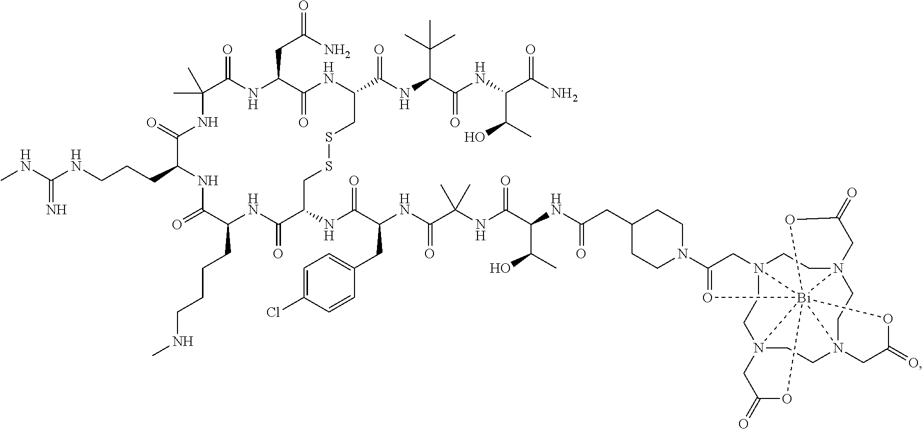

compound LaDOTA-Cmp-Thr-Aib-Pcf-[Cys-Lys(Me)-Arg(Me)-Aib-Asn-Cys]-Tle-Thr-NH₂ (PSM-0596) of the following formula

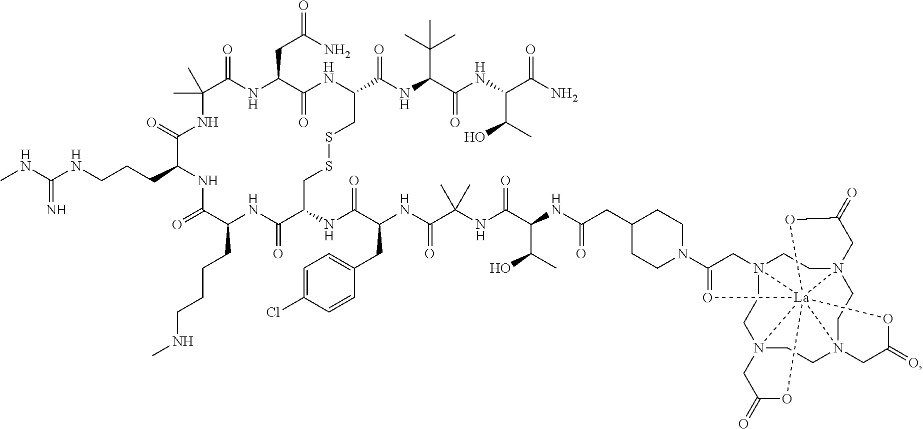

compound PbDOTA-Cmp-Thr-Aib-Pcf-[Cys-Lys(Me)-Arg(Me)-Aib-Asn-Cys]-Tle-Thr-NH₂ (PSM-0597) of the following formula

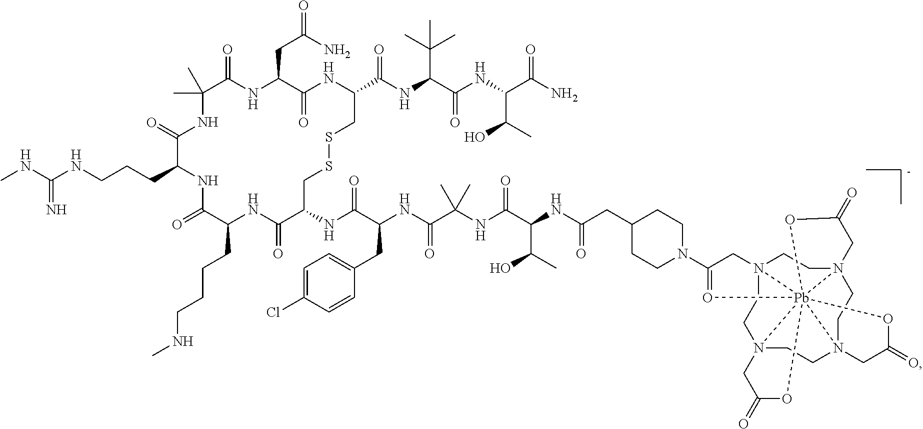

compound BiDOTAM-Cmp-Thr-Aib-Pcf-[Cys-Lys(Me)-Arg(Me)-Aib-Asn-Cys]-Tle-Thr-NH₂ (PSM-0598) of the following formula

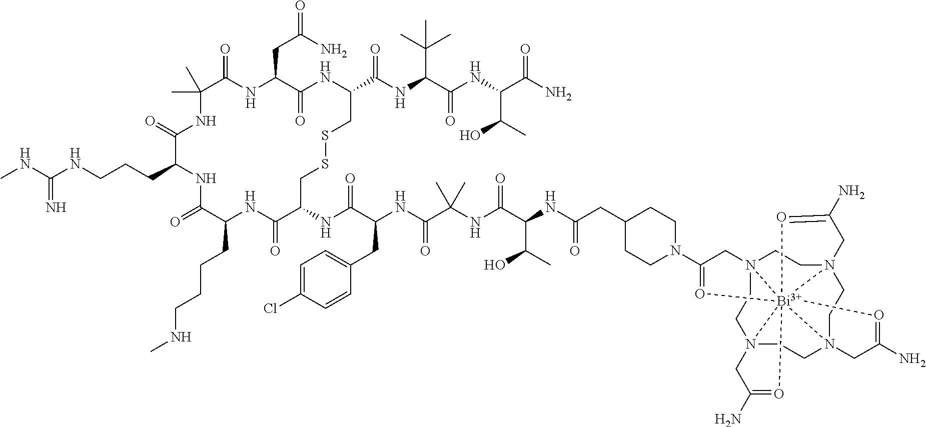

compound InDOTAM-Cmp-Thr-Aib-Pcf-[Cys-Lys(Me)-Arg(Me)-Aib-Asn-Cys]-Tle-Thr-NH₂ (PSM-0599) of the following formula

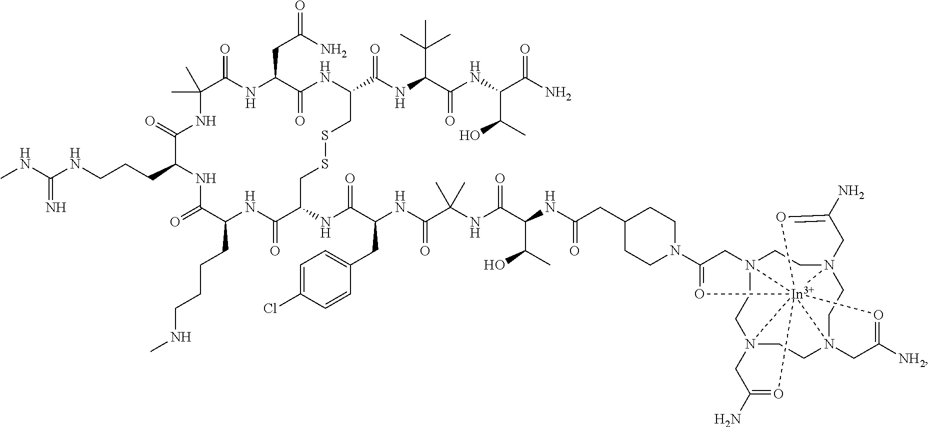

compound PbDOTAM-Cmp-Thr-Aib-Pcf-[Cys-Lys(Me)-Arg(Me)-Aib-Asn-Cys]-Tle-Thr-NH₂ (PSM-0600) of the following formula

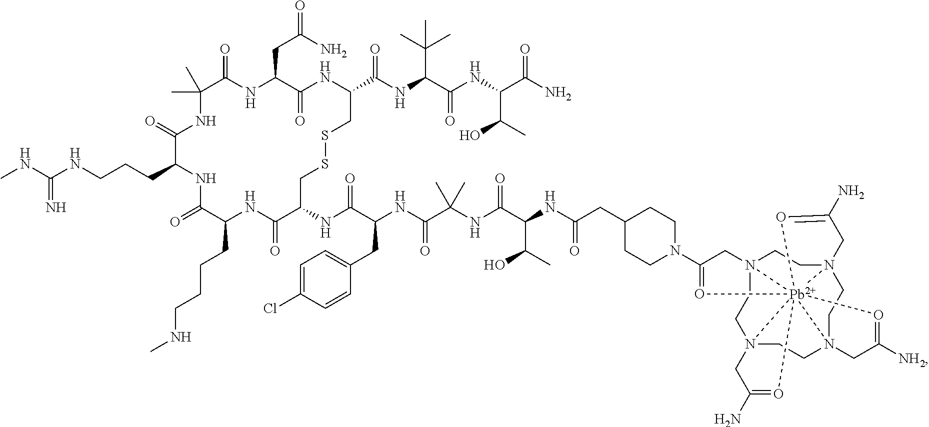

compound LSC-Cmp-Thr-Aib-Pcf-[Cys-Lys(Me)-Arg(Me)-Aib-Aan-Cys]-Tle-Thr-NH (PSM-0601) of the following formula

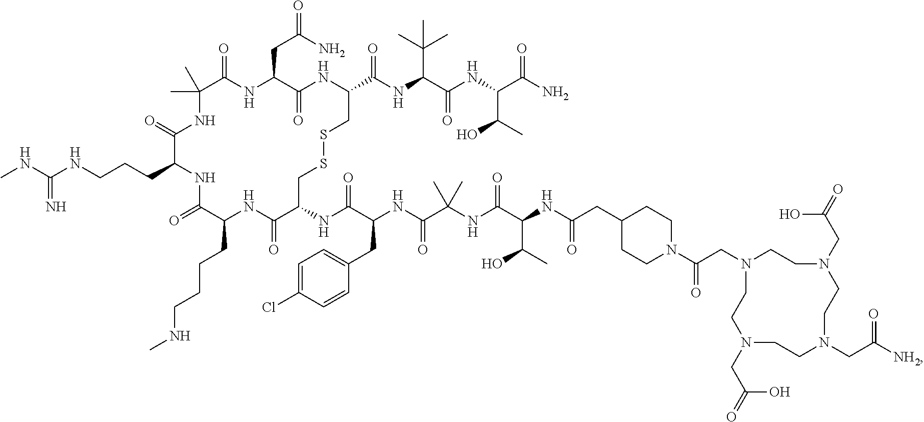 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(N)=O)CCN(CC(=O)O)CC3)CC2)[C@@H](C)O)CSSC[C@@H](C(=O)N[C@H](C(=O)N[C@H](C(N)=O)[C@@H](C)O)C(C)(C)C)NC(=O)[C@H](CC(N)=O)NC(=O)C(C)(C)NC(=O)[C@H](CCCNC(=N)NC)NC1=O]

compound PbLSC-Cmp-Thr-Aib-Pcf-[Cys-Lys(Me)-Arg(Me)-Aib-Asn-Cys]-Tle-Thr-NH₂ (PSM-0602) of the following formula

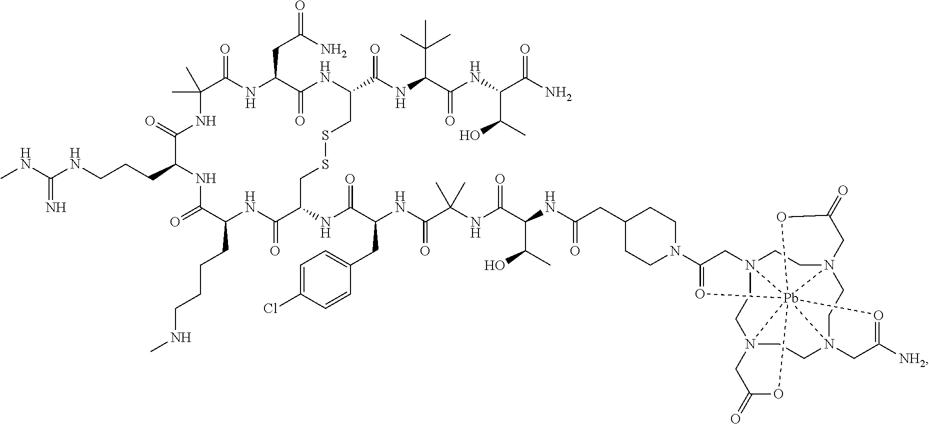

compound BiLSC-Cmp-Thr-Aib-Pcf-[Cys-Lys(Me)-Arg(Me)-Aib-Asn-Cys]-Tle-Thr-NH₂ (PSM-0603) of the following formula

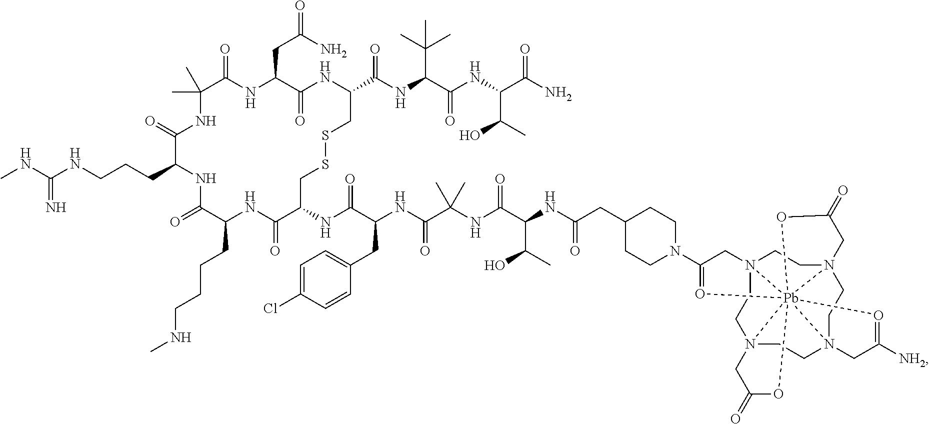

compound InLSC-Cmp-Thr-Aib-Pcf-[Cys-Lys(Me)-Arg(Me)-Aib-Asn-Cys]-Tle-Thr-NH₂ (PSM-0604) of the following formula

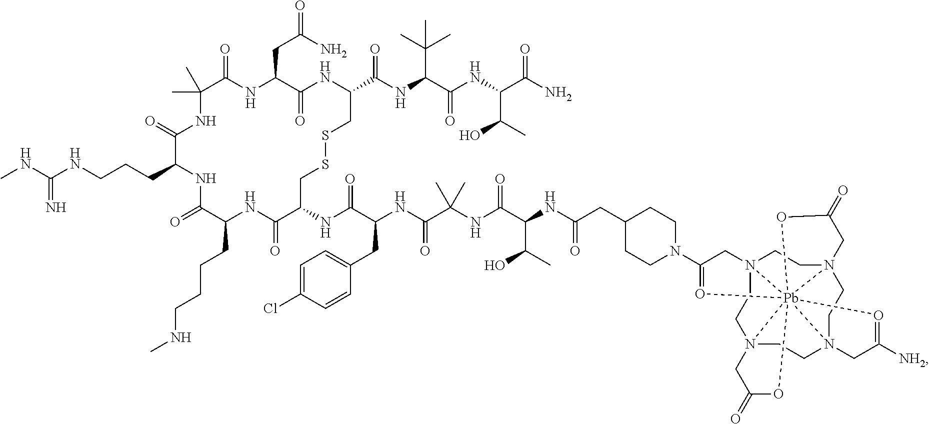

compound DOTA-Cmp-Thr-Aib-Phe-[Cys-Lys(Me)-Arg(Me)-Aib-Aan-Cys]-Tle-en (PSM-0605) of the following formula

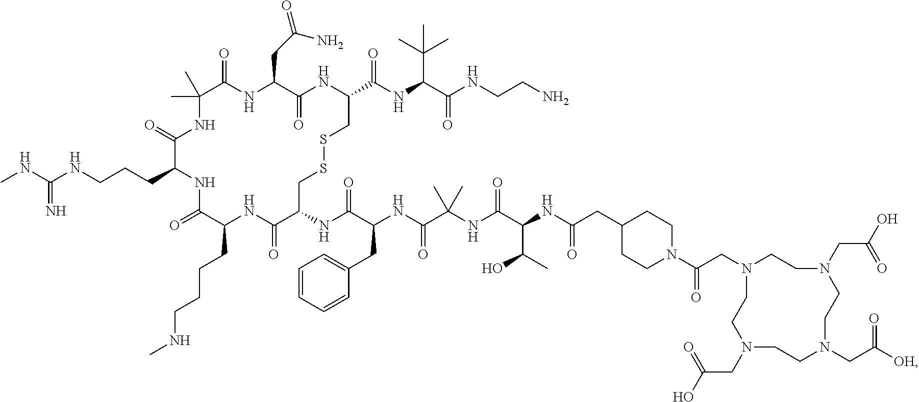 [SMILES: CNCCCC[C@@H]1NC(=O)[C@@H](NC(=O)[C@H](Cc2ccccc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)NCCN)C(C)(C)C)NC(=O)[C@H](CC(N)=O)NC(=O)C(C)(C)NC(=O)[C@H](CCCNC(=N)NC)NC1=O]

and compound DOTA-Cmp-Thr-Aib-Pcf-[Cys-Lys(Me)-Arg(Me)-Aib-Asn-Cys]-Tle-Bal-NH₂ (PSM-0606) of the following formula

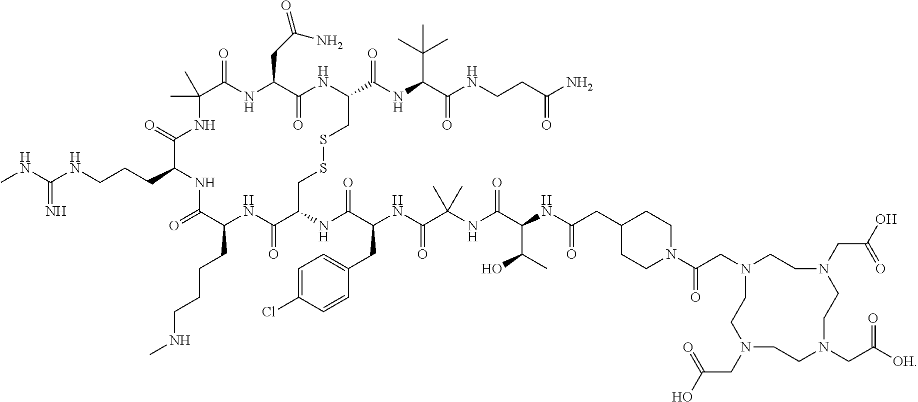 [SMILES: CNCCCC[C@@H]1NC(=O)[C@@H](NC(=O)[C@H](Cc2ccc(Cl)cc2)NC(=O)C(C)(C)NC(=O)[C@@H](NC(=O)CC2CCN(C(=O)CN3CCN(CC(=O)O)CCN(CC(=O)O)CCN(CC(=O)O)CC3)CC2)[C@@H](C)O)CSSC[C@@H](C(=O)N[C@H](C(=O)NCCC(N)=O)C(C)(C)C)NC(=O)[C@H](CC(N)=O)NC(=O)C(C)(C)NC(=O)[C@H](CCCNC(=N)NC)NC1=O]

### Example 31: FACS Binding Assay

In order to determine binding of compounds according to the present invention to PSMA-expressing cells, a FACS binding assay was established.

PSMA-expressing C4-2 cells (ATCC, Cat. No. CRL-3314) were cultured in RPMI-1640 (Pan Biotech, Cat. No. P04-18050) including 10% fetal calf serum (Biochrom) and 100 U/ml penicillin and 100 μg/mL streptomycin (Sigma, Cat. No. P0781). Cells were detached with Accutase (Biolegend, Cat. No. BLD-423201) and washed in FACS buffer (PBS (Sigma, Cat. No. D8537) including 1% fetal calf serum). Cells were diluted in FACS buffer to a final concentration of 500,000 cells per mL. 200 μL of the cell suspension were transferred to a u-shaped non-binding 96-well plate (Greiner Bio-One, Cat. No. 650901) and cells were washed in ice-cold FACS buffer.

For IC₅₀ determination, C4-2 cells were incubated with 50 nM PSM-0183 (Ac-Thr-Aib-Phe[Cys-Lys-Arg-Aib-Asn-Cys]-Tle-Thr-Asp-Ttds-Ttds-AF488N3K-NH₂) in the presence of increasing concentrations of test compounds at 4° C. for 1 hour. After two wash steps with ice-cold FACS buffer, cells were analyzed in an Attune NxT flow cytometer. Median fluorescence intensities (MFI) of the FITC/AF488-channel were calculated by Attune NxT software. MFI values were plotted against peptide concentration and four parameter logistic (4PL) curve fitting and IC₅₀/pIC₅₀ calculations were performed using ActivityBase software (IDBS).

The results of the IC₅₀ assay are shown in Table 6. pIC₅₀ category A stands for pIC₅₀ values >6.7, category B for pIC₅₀ values from >5.9 to 6.7, category C for pIC₅₀ values from 5.5 to 5.9, and category D for pIC₅₀ values <5.5.

### Example 32: Surface Plasmon Resonance Assay

Surface plasmon resonance (SPR) studies were performed using a Biacore™ T200 SPR system. Briefly, polarized light is directed towards a gold-labeled sensor surface, and minimum intensity reflected light is detected. The angle of reflected light changes as molecules bind and dissociate.

Fc-fusion protein of human PSMA (hPSMA-Fc, Acro Biosystems, Cat #PSA-H5264) was captured on a Fc-capture chip (Biacore™ CM5 sensor chip coated with ˜300 RU of an Fc-binding peptide). hPSMA-Fc was diluted in Running Buffer (HBST, 0.1% DMSO) to a final concentration of 100 nM and then flushed over the Fc-capture chip to immobilized ˜1000 RUs.

Stock solutions of test compounds were prepared by dissolving each compound in DMSO. DMSO stock solution were diluted 1:1000 in Running Buffer without DMSO. Further sequential dilutions were made with Running Buffer containing 0.1% DMSO. SPR binding analyses were performed in Single Cycle Kinetic (SCK) mode at 25° C. Flow cell coated with the Fc-binding peptide only served as reference flowcell. After each SCK run, hPSMA-Fc was removed with 10 mM glycine buffer, pH 1.5.

In between every three SCK measurements, a blank run with Running Buffer instead of test compound was included to correct for baseline drifts (double blanking method).

Table 5 describes the protocol steps for Fc-fusion target capturing and assessment of the binding kinetics.

TABLE 5
SPR protocol steps with hPSMA-Fc.

| | Injected | | | Flow |
|---|---|---|---|---|
| Step | solution | Contact time | | rate |
| Startup | HBST, 0.1% | 60 | s | 30 μL/min |
| cycle (3x): | DMSO Buffer | | | |
| Washing & surface | 10 mM glycine, | 5 | s | |
| regeneration | pH 1.5 | | | |
| Caputure target | 100 nM hPSMA-Fc | 500 | s | 5 μL/min |
| protein | | | | |
| 1. Binding kinetics | Dilution no. 3 | 120 | s | 30 μL/min |
| of test compound | (e.g. 0.2 nM) | | | |
| 2. Binding kinetics | Dilution no. 2 | 120 | s | 30 μL/min |
| of test compound | (e.g. 0.8 nM) | | | |
| 3. Binding kinetics | Dilution no. 1 | 120 | s | 30 μL/min |
| of test compound | (e.g. 3.1 nM) | | | |
| Dissociation cycle | HBST, 0.1% | 1200 | s | 30 μL/min |
| | DMSO Buffer | | | |
| Regeneration (2x) | 10 mM glycine, | 20 | s | 5 μL/min |
| | pH 1.5 | | | |

For each test compound, SPR raw data in the form of resonance units (RU) were plotted as sensorgrams using the Biacore™ T200 control software. The signal from the blank sensorgram was subtracted from that of the test compound sensorgram (blank corrected). The blank corrected sensorgram was corrected for baseline drift by subtracting the sensorgram of a SCK run without the test compound (running buffer only). The dissociation constant (K_{D}) was calculated from Blank-normalized SPR data using the 1:1 Langmuir binding model from the Biacore™ Insight Evaluation software. The pK_{D} value (negative decadic logarithm of dissociation constant) was calculated using Microsoft Excel.

The results of this assay for a selection of compounds according to the present invention are presented in Table 6. pK_{D} category A stands for pK_{D} values >7.9, category B for pK_{D} values from >6.9 to 7.9, category C for pK_{D} values from 6.3 to 6.9, and category D for pK_{D} values <6.3.

TABLE 6
Compound ID, sequence, synthesis (described in Examples 2-29), exact
calculated mass, LC/TOF-MS exact mass found, retention time (Rₜ) in minutes as
determined by HPLC, pIC₅₀ category of FACS binding, and pKD category for PSMA
SPR activity assay

| | | Syn- | | | | pIC₅₀ | |
|---|---|---|---|---|---|---|---|
| Compound | | the- | Calc. | Found | Rₜ | [C4-2] | pK_{D} |
| ID | Sequence | sis | Mass | Mass | [min] | CAT | CAT |
| PSM-0178 | Ac-Thr-Aib-Phe-[Cys- | I | 1293.632 | 1293.640 | 4.33 | A | A |
| | Lys-Arg-Aib-Asn-Cys]- | | | | | | |
| | Tle-Thr-NH₂ | | | | | | |
| PSM-0179 | Ac-Thr-Aib-Pcf-[Cys- | I | 1328.577 | 1328.679 | 4.91 | A | A |
| | Lys-Cit-Aib-Asn-Cys]- | | | | | | |
| | Tle-Thr-NH₂ | | | | | | |
| PSM-0180 | Ac-Thr-Aib-Phe-[Cys- | I | 1366.601 | 1366.627 | 4.25 | A | B |
| | Lys-Gln-Ala-Asn-Cys]- | | | | | | |
| | Tle-Thr-Asp-NH₂ | | | | | | |
| PSM-0181 | Ac-Thr-Aib-Phe-[Cys- | I | 1436.654 | 1436.677 | 3.98 | A | A |
| | Lys-Arg-Hyp-Asn-Cys]- | | | | | | |
| | Tle-Thr-Asp-NH₂ | | | | | | |
| PSM-0182 | Ac-Thr-Ala-Phe-[Cys- | I | 1380.628 | 1380.688 | 4.33 | C | D |
| | Lys-Arg-Ala-Asn-Cys]- | | | | | | |
| | Nle-thr-Asp-NH₂ | | | | | | |
| PSM-0183 | Ac-Thr-Aib-Phe-[Cys- | XIII | 2959.233 | 2959.299 | 6.31 | n.d. | A |
| | Lys-Arg-Aib-Asn-Cys]- | | | | | | |
| | Tle-Thr-Asp-Ttds-Ttds- | | | | | | |
| | AF488N3K-NH₂ | | | | | | |
| PSM-0184 | DOTA-Thr-Aib-Phe- | I | 1536.754 | 1536.750 | 3.89 | A | B |
| | [Cys-Lys-Arg-Aib-Asn- | | | | | | |
| | Cys]-Tle-NH₂ | | | | | | |
| PSM-0185 | LuDOTA-Cmp-Thr- | XXb | 1895.748 | 1895.744 | 4.59 | A | A |
| | Aib-Pcf-[Cys-Lys(Me)- | | | | | | |
| | Arg(Me)-Aib-Asn-Cys]- | | | | | | |
| | Tle-NH₂ | | | | | | |
| PSM-0186 | Ac-Pamp-Aib-Pcf-[Cys- | VI | 1688.768 | 1688.753 | 4.12 | A | A |
| | Lys-Arg-Aib-Asn-Cys]- | | | | | | |
| | Tle-en-DOTA | | | | | | |
| PSM-0187 | Ac-Thr-Deg-Phe-[Cys- | I | 1321.664 | 1321.769 | 4.68 | A | A |
| | Lys-Arg-Aib-Asn-Cys]- | | | | | | |
| | Tle-Thr-NH₂ | | | | | | |
| PSM-0188 | SaPr-Aib-Pcf-[Cys-Lys- | II | 1888.851 | 1888.844 | 4.32 | A | A |
| | Arg-Lys(DOTA-APAc)- | | | | | | |
| | Asn-Cys]-Tle-Thr-NH₂ | | | | | | |
| PSM-0189 | Ac-Thr-Aib-Pcf-[Cys- | IX | 1341.573 | 1341.548 | 4.77 | A | A |
| | Lys-Gln(Gu)-Aib-Asn- | | | | | | |
| | Cys]-Tle-Thr-NH₂ | | | | | | |
| PSM-0190 | Ac-Thr-Aib-Pcf-[Cys- | III | 1966.952 | 1966.982 | 4.72 | A | A |
| | Lys-Arg-Aib-Asn-Cys]- | | | | | | |
| | Tle-Thr-lys(DOTA- | | | | | | |
| | Cmp)-NH₂ | | | | | | |
| PSM-0191 | Ac-Thr-Aib-Phe-[Cys- | II | 1948.002 | 1948.115 | 3.98 | A | A |
| | Lys-Arg-Aib-Asn-Cys]- | | | | | | |
| | Tle-Thr-APAc- | | | | | | |
| | lys(DOTA)-NH₂ | | | | | | |
| PSM-0192 | H-Cmp-Thr-Aib-Miy- | I | 1546.629 | 1546.634 | 2.84 | D | D |
| | [Cys-Lys(Me)-Arg(Me)- | | | | | | |
| | Aib-Asn-Cys]-Tle-Thr- | | | | | | |
| | NH₂ | | | | | | |
| PSM-0193 | DOTA-Ttds-Thr-Aib- | I | 2040.997 | 2041.063 | 4.09 | A | A |
| | Phe-[Cys-Lys-Arg-Ala- | | | | | | |
| | Asn-Cys]-Tle-Thr-Asp- | | | | | | |
| | NH₂ | | | | | | |
| PSM-0194 | DOTA-Cmp-Thr-Aib- | I | 1824.878 | 1824.884 | 4.19 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0195 | InDOTA-Cmp-Thr-Aib- | XXa | 1872.766 | 1872.779 | 4.53 | A | A |
| | Phe-[Cys-Lys-Arg-Aib- | | | | | | |
| | Asn-Cys]-Tle-Thr-NH₂ | | | | | | |
| PSM-0196 | GaDOTA-Ttds-Thr- | XXc | 1991.872 | 1991.870 | 4.02 | A | A |
| | Aib-Phe-[Cys-Lys-Arg- | | | | | | |
| | Ala-Asn-Cys]-Tle-Thr- | | | | | | |
| | NH₂ | | | | | | |
| PSM-0197 | Ac-Thr-Ala-Phe-[Cys- | I | 1438.633 | 1438.643 | 4.21 | A | B |
| | Lys-Arg-Glu-Asn-Cys]- | | | | | | |
| | Nle-Thr-Asp-NH₂ | | | | | | |
| PSM-0198 | DOTA-Cmp-Tle-Aib- | I | 1774.922 | 1774.926 | 4.16 | C | C |
| | Phe-[Cys-Lys-Arg-Aib- | | | | | | |
| | Asn-Cys]-Tle-Thr-NH₂ | | | | | | |
| PSM-0199 | DOTA-Thr-Aib-Phe- | V | 1579.796 | 1579.793 | 3.50 | A | A |
| | [Cys-Lys-Arg-Aib-Asn- | | | | | | |
| | Cys]-Tle-en | | | | | | |
| PSM-0200 | DOTA-Cmp-Thr-Aib- | I | 1709.815 | 1709.818 | 3.84 | A | A |
| | Pcf-[Cys-Lys(Me)-Arg- | | | | | | |
| | Aib-Asn-Cys]-Tle-NH₂ | | | | | | |
| PSM-0201 | DOTA-Ttds-Thr-Ala- | I | 1697.823 | 1697.839 | 3.64 | D | D |
| | Phe-[Cys-Lys-Arg-Ala- | | | | | | |
| | Asn-Cys]-NH₂ | | | | | | |
| PSM-0202 | DOTA-Pamb-Thr-Aib- | I | 1770.855 | 1770.951 | 4.24 | A | A |
| | Phe-[Cys-Lys-Arg-Aib- | | | | | | |
| | Asn-Cys]-Tle-Thr-NH₂ | | | | | | |
| PSM-0203 | DOTA-Cmp-Thr-Aib- | I | 1838.894 | 1839.001 | 3.60 | A | A |
| | Pcf-[Cys-KMe₂- | | | | | | |
| | Arg(Me)-Aib-Asn-Cys]- | | | | | | |
| | Tle-Thr-NH₂ | | | | | | |
| PSM-0204 | nBuCAyl-Thr-Aib-Pcf- | IV | 2024.994 | 2024.994 | 6.07 | A | B |
| | [Cys-Lys-Cit-Aib-Asn- | | | | | | |
| | Cys]-Tle-Thr-Cmp- | | | | | | |
| | lys(DOTA)-NH₂ | | | | | | |
| PSM-0205 | Ac-Thr-Aib-Pcf-[Cys- | I | 1312.582 | 1312.682 | 6.09 | A | A |
| | Nle-Arg-Aib-Asn-Cys]- | | | | | | |
| | Tle-Thr-NH₂ | | | | | | |
| PSM-0206 | DOTA-Cmp-Thr-Aib- | I | 1907.839 | 1907.834 | 0.00 | C | D |
| | 5Brw-[Cys-Lys(Me)- | | | | | | |
| | Arg(Me)-Aib-Asn-Cys]- | | | | | | |
| | Tle-Thr-NH₂ | | | | | | |
| PSM-0207 | nBuCAyl-Thr-Aib-Mcf- | IV | 2024.994 | 2024.998 | 6.02 | A | B |
| | [Cys-Lys-Cit-Aib-Asn- | | | | | | |
| | Cys]-Tle-Thr-Cmp- | | | | | | |
| | lys(DOTA)-NH₂ | | | | | | |
| PSM-0208 | Ac-Thr-Aib-Phe-[Cys- | XI | 1149.579 | 1149.578 | 5.45 | B | C |
| | Lys-Arg-Aib-Asn-Cys]- | | | | | | |
| | NHnPen | | | | | | |
| PSM-0209 | DOTA-Bal-Thr-Aib- | I | 1708.839 | 1708.938 | 3.87 | A | A |
| | Phe-[Cys-Lys-Arg-Aib- | | | | | | |
| | Asn-Cys]-Tle-Thr-NH₂ | | | | | | |
| PSM-0210 | Ac-Thr-Aib-Phe-[Cys- | I | 1408.623 | 1408.629 | 4.60 | A | B |
| | Gln-Arg-Aib-Asn-Cys]- | | | | | | |
| | Tle-Thr-Asp-NH₂ | | | | | | |
| PSM-0211 | Ac-Thr-Aib-Phe-[Cys- | III | 1841.892 | 1842.003 | 4.16 | A | A |
| | Lys-Arg-Lys(DOTA- | | | | | | |
| | Pab)-Asn-Cys]-Tle-Thr- | | | | | | |
| | NH₂ | | | | | | |
| PSM-0212 | Ac-Thr-Aib-Phe-[Cys- | II | 2225.118 | 2225.141 | 4.52 | A | A |
| | Lys-Arg-Aib-Asn-Cys]- | | | | | | |
| | Tle-Thr-Asp-Ttds- | | | | | | |
| | lys(DOTA)-NH₂ | | | | | | |
| PSM-0213 | LuDOTA-Cmp-Thr- | XXb | 1934.803 | 1934.817 | 4.44 | A | A |
| | Aib-Phe-[Cys-Lys-Arg- | | | | | | |
| | Aib-Asn-Cys]-Tle-Thr- | | | | | | |
| | NH₂ | | | | | | |
| PSM-0214 | nBuCAyl-Thr-Aib-Phe- | XXa | 2099.930 | 2099.936 | 5.75 | A | A |
| | [Cys-Lys-Arg-Aib-Asn- | | | | | | |
| | Cys]-Tle-Thr-Cmp- | | | | | | |
| | lys(InDOTA)-NH₂ | | | | | | |
| PSM-0215 | SaPr-Aib-Pcf-[Cys-Lys- | II | 1958.893 | 1958.892 | 4.58 | A | B |
| | Arg-Aib-Asn-Cys]-Tle- | | | | | | |
| | Thr-Cmp-lys(DOTA)- | | | | | | |
| | NH₂ | | | | | | |
| PSM-0216 | DOTA-Thr-Aib-Pcf- | I | 1641.752 | 1641.755 | 4.17 | A | A |
| | [Cys-Lys-Arg-Aib-Asn- | | | | | | |
| | Cys]-Tle-Bal-NH₂ | | | | | | |
| PSM-0217 | Ac-Thr-Aib-Phe-[Cys- | I | 1456.640 | 1456.673 | 4.33 | A | B |
| | Lys-Arg-Dfp-Asn-Cys]- | | | | | | |
| | Tle-Thr-Asp-NH₂ | | | | | | |
| PSM-0218 | DOTAGA-Cmp-Thr- | I | 1834.907 | 1834.969 | 4.15 | A | A |
| | Aib-Phe-[Cys-Lys-Arg- | | | | | | |
| | Aib-Asn-Cys]-Tle-Thr- | | | | | | |
| | NH₂ | | | | | | |
| PSM-0220 | DOTA-Cmp-Leu-Aib- | I | 1774.922 | 1774.924 | 5.11 | A | B |
| | Phe-[Cys-Lys-Arg-Aib- | | | | | | |
| | Asn-Cys]-Tle-Thr-NH₂ | | | | | | |
| PSM-0221 | DOTA-Pamb-Aib-Phe- | V | 1611.801 | 1611.799 | 4.00 | A | A |
| | [Cys-Lys-Arg-Aib-Asn- | | | | | | |
| | Cys]-Tle-en | | | | | | |
| PSM-0222 | DOTA-Thr-Aib-Pcf- | V | 1613.757 | 1613.758 | 4.01 | A | A |
| | [Cys-Lys-Arg-Aib-Asn- | | | | | | |
| | Cys]-Tle-en | | | | | | |
| PSM-0223 | DOTA-Cmp-Aib-Pcf- | X | 1737.810 | 1737.806 | 4.59 | B | B |
| | [Cys-Lys-Arg(Ac)-Aib- | | | | | | |
| | Asn-Cys]-Tle-Thr-NH₂ | | | | | | |
| PSM-0224 | SaPr-Aib-Phe-[Cys-Lys- | I | 1285.573 | 1285.549 | 4.05 | A | A |
| | Arg-Aib-Asn-Cys]-Tle- | | | | | | |
| | Thr-NH₂ | | | | | | |
| PSM-0225 | DOTA-Cmp-Thr-Aib- | I | 1838.869 | 1839.001 | 4.22 | A | A |
| | Pcf-[Cys-Har-Arg-Aib- | | | | | | |
| | Asn-Cys]-Tle-Thr-NH₂ | | | | | | |
| PSM-0226 | H-Met-Thr-Ala-Phe- | I | 2874.436 | 2874.472 | 4.85 | B | n.d. |
| | [Cys-Lys-Arg-Ala-Asn- | | | | | | |
| | Cys]-Leu-Thr-Asp-Gly- | | | | | | |
| | Ser-Ttds-Ttds-Ttds- | | | | | | |
| | Lys(Bio)-NH₂ | | | | | | |
| PSM-0227 | DOTA-Cmp-Thr-Aib- | I | 1840.837 | 1841.001 | 3.96 | A | A |
| | Pcf-[Cys-Lys-Arg-amd- | | | | | | |
| | Asn-Cys]-Tle-Thr-NH₂ | | | | | | |
| PSM-0228 | Ac-Aib-Phe-[Cys-Lys- | I | 1192.585 | 1192.563 | 4.21 | A | A |
| | Arg-Aib-Asn-Cys]-Tle- | | | | | | |
| | Thr-NH₂ | | | | | | |
| PSM-0229 | Ac-Thr-Aib-Pcf-[Cys- | I | 1344.591 | 1344.591 | 4.36 | n.d. | A |
| | Lys-Arg-Aib-Met-Cys]- | | | | | | |
| | Tle-Thr-NH₂ | | | | | | |
| PSM-0230 | Ac-Thr-Aib-Phe-[Cys- | I | 1192.585 | 1192.583 | 4.55 | A | A |
| | Lys-Arg-Aib-Asn-Cys]- | | | | | | |
| | Tle-NH₂ | | | | | | |
| PSM-0231 | Ac-Thr-Aib-Phe-[Cys- | II | 2096.076 | 2096.238 | 4.57 | A | A |
| | Lys-Arg-Ala-Asn-Cys]- | | | | | | |
| | Tle-Thr-Ttds- | | | | | | |
| | lys(DOTA)-NH₂ | | | | | | |
| PSM-0232 | Ac-Thr-Aib-Phe-[Cys- | I | 1279.617 | 1279.615 | 4.18 | A | A |
| | Lys-Arg-Nmg-Asn- | | | | | | |
| | Cys]-Tle-Thr-NH₂ | | | | | | |
| PSM-0233 | Ac-Thr-Aib-Phe-[Cys- | I | 1319.648 | 1319.625 | 4.37 | A | A |
| | Lys-Arg-Pam-Asn- | | | | | | |
| | Cys]-Tle-Thr-NH₂ | | | | | | |
| PSM-0234 | DOTA-Ttds-Thr-Aib- | I | 1940.970 | 1941.076 | 4.51 | B | B |
| | Phe-[Cys-Lys-Cit-Aib- | | | | | | |
| | Asn-Cys]-Tle-Thr-NH₂ | | | | | | |
| PSM-0235 | Ac-Thr-Aib-Phe-[Cys- | I | 1307.648 | 1307.646 | 4.80 | B | C |
| | Lys-Nmr-Aib-Asn- | | | | | | |
| | Cys]-Tle-Thr-NH₂ | | | | | | |
| PSM-0236 | Ac-Thr-Aib-Pcf-[Cys- | IX | 1326.562 | 1326.561 | 6.41 | A | A |
| | Nle-Gln(Gu)-Aib-Asn- | | | | | | |
| | Cys]-Tle-Thr-NH₂ | | | | | | |
| PSM-0237 | DOTA-Ttds-Thr-Aib- | I | 1925.970 | 1925.972 | 4.05 | A | A |
| | Phe-[Cys-Lys-Arg-Ala- | | | | | | |
| | Asn-Cys]-Tle-Thr-NH₂ | | | | | | |
| PSM-0238 | Ac-Thr-Aib-Pcf-[Cys- | I | 1400.614 | 1400.648 | 5.98 | A | A |
| | Lys-Cit-Aib-Trp-Cys]- | | | | | | |
| | Tle-Thr-NH₂ | | | | | | |
| PSM-0239 | Hex-Thr-Aib-Pcf-[Cys- | III | 2023.999 | 2024.012 | 6.12 | A | A |
| | Lys-Cit-Aib-Asn-Cys]- | | | | | | |
| | Tle-Thr-lys(DOTA- | | | | | | |
| | Cmp)-NH₂ | | | | | | |
| PSM-0240 | Ac-Thr-Aib-Pcf-[Cys- | I | 1370.635 | 1370.677 | 4.64 | A | A |
| | Lys-Cit-Aib-Arg-Cys]- | | | | | | |
| | Tle-Thr-NH₂ | | | | | | |
| PSM-0241 | DOTA-Cmp-Thr-Aib- | V | 1704.880 | 1704.875 | 3.66 | A | A |
| | Phe-[Cys-Lys-Arg-Aib- | | | | | | |
| | Asn-Cys]-Tle-en | | | | | | |
| PSM-0243 | Ac-Thr-Aib-Mcf-[Cys- | III | 1901.890 | 1902.017 | 4.44 | A | A |
| | Lys-Arg-Lys(DOTA- | | | | | | |
| | O₂0c)-Asn-Cys]-Tle- | | | | | | |
| | Thr-NH₂ | | | | | | |
| PSM-0244 | DOTA-Cmp-Thr-Aib- | I | 1852.910 | 1852.906 | 3.66 | A | A |
| | Pcf-[Cys-Kip-Arg(Me)- | | | | | | |
| | Aib-Asn-Cys]-Tle-Thr- | | | | | | |
| | NH₂ | | | | | | |
| PSM-0245 | DOTA-Cmp-Thr-Aib- | I | 1754.825 | 1754.825 | 4.09 | A | A |
| | Pcf-[Cys-Lys-Orn-Aib- | | | | | | |
| | Asn-Cys]-Tle-Thr-NH₂ | | | | | | |
| PSM-0246 | DOTA-Cmp-Thr-Aib- | I | 1810.863 | 1810.860 | 3.19 | A | A |
| | Pcf-[Cys-Lys(Me)- | | | | | | |
| | Arg(Me)-ala-Asn-Cys]- | | | | | | |
| | Tle-Thr-NH₂ | | | | | | |
| PSM-0247 | DOTA-Cmp-Thr-Aib- | I | 1838.894 | 1839.000 | 3.61 | A | A |
| | Pcf-[Cys-Lys(Me)- | | | | | | |
| | RMe₂-Aib-Asn-Cys]- | | | | | | |
| | Tle-Thr-NH₂ | | | | | | |
| PSM-0248 | DOTA-Cmp-Aib-Pcf- | I | 1695.799 | 1695.806 | 4.42 | A | B |
| | [Cys-Lys-Arg-Aib-Asn- | | | | | | |
| | Cys]-Tle-Thr-NH₂ | | | | | | |
| PSM-0249 | Ac-Thr-Aib-1Ni-[Cys- | I | 1458.675 | 1458.769 | 5.11 | A | A |
| | Lys-Arg-Aib-Asn-Cys]- | | | | | | |
| | Tle-Thr-Asp-NH₂ | | | | | | |
| PSM-0250 | DOTA-Cmp-Thr-Aib- | I | 2755.388 | 2755.381 | 5.08 | B | A |
| | Pcf-[Cys-Lys-Arg-Aib- | | | | | | |
| | Asn-Cys]-Tle-Thr-Ttds- | | | | | | |
| | Ttds-Lys(Bio)-NH₂ | | | | | | |
| PSM-0251 | DOTA-Ttds-Thr-Ala- | I | 2026.982 | 2027.007 | 4.17 | B | n.d. |
| | Phe-[Cys-Lys-Arg-Ala- | | | | | | |
| | Asn-Cys]-Leu-Thr-Asp- | | | | | | |
| | NH₂ | | | | | | |
| PSM-0252 | Ac-Thr-Aib-Phe-[Cys- | II | 1933.975 | 1933.967 | 4.28 | B | D |
| | Lys-Cit-Aib-Asn-Cys]- | | | | | | |
| | Tle-Thr-Cmp- | | | | | | |
| | lys(DOTA)-NH₂ | | | | | | |
| PSM-0253 | Ac-Thr-Aib-Phe-[Cys- | I | 1381.601 | 1381.608 | 4.52 | B | C |
| | Lys-Glu-Aib-Asn-Cys]- | | | | | | |
| | Tle-Thr-Asp-NH₂ | | | | | | |
| PSM-0254 | DOTA-Cmp-Thr-Aib- | I | 1855.848 | 1855.859 | 3.77 | A | A |
| | Pcf-[Cys-Lys(Me)-Eew- | | | | | | |
| | Aib-Asn-Cys]-Tle-Thr- | | | | | | |
| | NH₂ | | | | | | |
| PSM-0255 | DOTA-Cmp-Thr-Aib- | I | 1764.865 | 1764.857 | 3.30 | C | D |
| | Mpa-[Cys-Lys-Cit-Aib- | | | | | | |
| | Asn-Cys]-Tle-Thr-NH₂ | | | | | | |
| PSM-0256 | DOTA-Cmp-Thr-ams- | I | 1812.842 | 1812.853 | 4.03 | A | A |
| | Pcf-[Cys-Lys-Arg-Aib- | | | | | | |
| | Asn-Cys]-Tle-Thr-NH₂ | | | | | | |
| PSM-0257 | DOTA-Cmp-Thr-Aib- | I | 1791.912 | 1791.910 | 2.41 | B | B |
| | Opa-[Cys-Lys(Me)- | | | | | | |
| | Arg(Me)-Aib-Asn-Cys]- | | | | | | |
| | Tle-Thr-NH₂; | | | | | | |
| PSM-0258 | nBuCAyl-Thr-Aib-Phe- | IV | 2025.054 | 2025.051 | 5.51 | B | C |
| | [Cys-Lys-Opy-Aib-Asn- | | | | | | |
| | Cys]-Tle-Thr-Cmp- | | | | | | |
| | lys(DOTA)-NH₂ | | | | | | |
| PSM-0259 | DOTA-Ttds-Thr-Aib- | I | 1939.986 | 1940.069 | 4.31 | A | A |
| | Phe-[Cys-Lys-Arg-Aib- | | | | | | |
| | Asn-Cys]-Tle-Thr-NH₂ | | | | | | |
| PSM-0260 | DOTA-Cmp-Thr-Aib- | I | 1840.837 | 1841.002 | 3.98 | A | A |
| | Pcf-[Cys-Lys-Arg-glu- | | | | | | |
| | Asn-Cys]-Tle-Thr-NH₂ | | | | | | |
| PSM-0261 | DOTA-APAc-Aib-Phe- | I | 1676.849 | 1676.921 | 3.49 | A | B |
| | [Cys-Lys-Arg-Aib-Asn- | | | | | | |
| | Cys]-Tle-Thr-NH₂ | | | | | | |
| PSM-0262 | Ac-Thr-Aib-Phe-[Cys- | I | 1321.627 | 1321.627 | 4.39 | A | A |
| | Lys-Arg-Aib-Asn-Cys]- | | | | | | |
| | Tle-Glu-NH₂ | | | | | | |
| PSM-0263 | DOTA-APAc-Aib-Pcf- | I | 1710.810 | 1710.805 | 4.23 | A | A |
| | [Cys-Lys-Arg-Aib-Asn- | | | | | | |
| | Cys]-Tle-Thr-NH₂ | | | | | | |
| PSM-0264 | Ac-Thr-Aib-Mtf-[Cys- | I | 1361.620 | 1361.618 | 4.66 | A | A |
| | Lys-Arg-Aib-Asn-Cys]- | | | | | | |
| | Tle-Thr-NH₂ | | | | | | |
| PSM-0265 | InDOTA-Ttds-Thr-Aib- | XXa | 2035.851 | 2035.844 | 4.32 | A | A |
| | Phe-[Cys-Lys-Arg-Ala- | | | | | | |
| | Asn-Cys]-Tle-Thr-NH₂ | | | | | | |
| PSM-0266 | Ac-Thr-Ala-Phe-[Cys- | II | 2197.087 | 2197.086 | 4.28 | A | B |
| | Lys-Arg-Ala-Asn-Cys]- | | | | | | |
| | Tle-Thr-Asp-Ttds- | | | | | | |
| | lys(DOTA)-NH₂ | | | | | | |
| PSM-0267 | HPA-Aib-Pcf-[Cys-Lys- | II | 1770.831 | 1770.827 | 4.43 | A | A |
| | Arg-Aib-Asn-Cys]-Tle- | | | | | | |
| | Thr-lys(DOTA)-NH₂ | | | | | | |
| PSM-0268 | Hex-Thr-Aib-Phe-[Cys- | XXa | 2391.062 | 2391.165 | 6.21 | A | A |
| | Lys-Arg-Aib-Asn-Cys]- | | | | | | |
| | Tle-Thr-Asp-Ttds- | | | | | | |
| | lys(InDOTA)-NH₂ | | | | | | |
| PSM-0269 | Ac-Thr-Aib-Pcf-[Cys- | VIII | 1655.768 | 1655.764 | 4.55 | A | A |
| | Lys-Arg-Aib-Asn-Cys]- | | | | | | |
| | Tle-en-DOTA | | | | | | |
| PSM-0270 | DOTA-Pamb-Aib-Phe- | I | 1669.807 | 1669.875 | 4.09 | A | A |
| | [Cys-Lys-Arg-Aib-Asn- | | | | | | |
| | Cys]-Tle-Thr-NH₂ | | | | | | |
| PSM-0272 | AF488Ahx-Ttds-Ttds- | XIII | 2902.211 | 2902.224 | 6.52 | n.d. | A |
| | Thr-Aib-Phe-[Cys-Lys- | | | | | | |
| | Arg-Aib-Asn-Cys]-Tle- | | | | | | |
| | Thr-Asp-NH₂ | | | | | | |
| PSM-0273 | DOTA-Pamb-Aib-Pcf- | V | 1645.762 | 1645.764 | 4.30 | A | A |
| | [Cys-Lys-Arg-Aib-Asn- | | | | | | |
| | Cys]-Tle-en | | | | | | |
| PSM-0274 | DOTA-Cmp-Thr-Aib- | I | 1831.792 | 1831.789 | 5.25 | A | B |
| | Eaa-[Cys-Lys-Cit-Aib- | | | | | | |
| | Asn-Cys]-Tle-Thr-NH₂ | | | | | | |
| PSM-0275 | Ac-Thr-Aib-Mcf-[Cys- | II | 1967.936 | 1968.067 | 4.77 | B | B |
| | Lys-Cit-Aib-Asn-Cys]- | | | | | | |
| | Tle-Thr-Cmp- | | | | | | |
| | lys(DOTA)-NH₂ | | | | | | |
| PSM-0276 | DOTA-Cmp-Thr-Glu- | I | 1840.837 | 1841.000 | 3.86 | D | D |
| | Pcf-[Cys-Lys-Arg-Aib- | | | | | | |
| | Asn-Cys]-Tle-Thr-NH₂ | | | | | | |
| PSM-0277 | InDOTA-Ttds-Thr-Aib- | XXa | 2150.878 | 2150.972 | 4.46 | A | A |
| | Phe-[Cys-Lys-Arg-Ala- | | | | | | |
| | Asn-Cys]-Tle-Thr-Asp- | | | | | | |
| | NH₂ | | | | | | |
| PSM-0278 | Ac-Thr-Aib-Pnf-[Cys- | I | 1318.627 | 1318.624 | 3.52 | A | A |
| | Lys-Arg-Aib-Asn-Cys]- | | | | | | |
| | Tle-Thr-NH₂ | | | | | | |
| PSM-0279 | Ac-Thr-Aib-Phe-[Cys- | I | 1266.621 | 1266.691 | 4.23 | A | A |
| | Lys-Arg-Aib-Ser-Cys]- | | | | | | |
| | Tle-Thr-NH₂ | | | | | | |
| PSM-0280 | Ac-Thr-Aib-Phe-[Cys- | I | 1409.643 | 1409.652 | 4.45 | A | A |
| | Lys-Cit-Aib-Asn-Cys]- | | | | | | |
| | Tle-Thr-Asp-NH₂ | | | | | | |
| PSM-0281 | Ac-Pcf-[Cys-Lys-Arg- | I | 1040.445 | 1040.444 | 3.69 | D | D |
| | Aib-Asn-Cys]-Tle-NH₂ | | | | | | |
| PSM-0282 | Ac-Aib-Pcf-[Cys-Lys- | II | 1795.863 | 1795.856 | 4.34 | A | A |
| | Arg-Lys(DOTA-APAc)- | | | | | | |
| | Asn-Cys]-Tle-Thr-NH₂ | | | | | | |
| PSM-0283 | DOTA-Pamb-Aib-Pcf- | I | 1703.768 | 1703.764 | 4.80 | A | A |
| | [Cys-Lys-Arg-Aib-Asn- | | | | | | |
| | Cys]-Tle-Thr-NH₂ | | | | | | |
| PSM-0284 | Ac-Thr-Aib-Mcf-[Cys- | III | 1902.874 | 1902.994 | 4.60 | A | B |
| | Lys-Cit-Lys(DOTA- | | | | | | |
| | O₂0c)-Asn-Cys]-Tle- | | | | | | |
| | Thr-NH₂ | | | | | | |
| PSM-0285 | Ac-Thr-Aib-Phe-[Cys- | II | 2110.092 | 2110.239 | 4.60 | A | A |
| | Lys-Arg-Aib-Asn-Cys]- | | | | | | |
| | Tle-Thr-Ttds- | | | | | | |
| | lys(DOTA)-NH₂ | | | | | | |
| PSM-0286 | Ac-Thr-Ala-Phe-[Cys- | I | 1363.638 | 1363.699 | 4.38 | D | D |
| | Lys-Arg-Ala-Pro-Cys]- | | | | | | |
| | Nle-Thr-Asp-NH₂ | | | | | | |
| PSM-0287 | Ac-Thr-Ala-Phe-[Cys- | I | 1380.628 | 1380.666 | 4.28 | A | B |
| | Lys-Arg-Ala-Asn-Cys]- | | | | | | |
| | Nle-Thr-Asp-NH₂ | | | | | | |
| PSM-0288 | Ac-Thr-Aib-Phe-[Cys- | II | 1807.907 | 1808.014 | 4.13 | A | A |
| | Lys-Arg-Aib-Asn-Cys]- | | | | | | |
| | Tle-Thr-lys(DOTA)- | | | | | | |
| | NH₂ | | | | | | |
| PSM-0289 | Hex-Thr-Aib-Phe-[Cys- | II | 2138.112 | 2138.262 | 6.05 | B | B |
| | Lys-Gln-Aib-Asn-Cys]- | | | | | | |
| | Tle-Thr-Ttds- | | | | | | |
| | lys(DOTA)-NH₂ | | | | | | |
| PSM-0290 | DOTA-Aib-Pcf-[Cys- | I | 1570.715 | 1570.722 | 4.09 | D | D |
| | Lys-Arg-Aib-Asn-Cys]- | | | | | | |
| | Tle-Thr-NH₂ | | | | | | |
| PSM-0291 | Ac-Thr-Aib-Phe-[Cys- | I | 1395.591 | 1395.617 | 4.51 | C | D |
| | Glu-Arg-Ala-Asn-Cys]- | | | | | | |
| | Tle-Thr-Asp-NH₂ | | | | | | |
| PSM-0292 | Iva-Aib-Phe-[Cys-Lys- | II | 1748.907 | 1748.810 | 5.58 | A | B |
| | Arg-Aib-Asn-Cys]-Tle- | | | | | | |
| | Thr-lys(DOTA)-NH₂ | | | | | | |
| PSM-0293 | Ac-Thr-Aib-Pcf-[Cys- | I | 1301.566 | 1301.600 | 4.96 | A | A |
| | Lys-Cit-Aib-Ser-Cys]- | | | | | | |
| | Tle-Thr-NH₂ | | | | | | |
| PSM-0294 | Ac-Thr-Aib-Pff-[Cys- | I | 1311.623 | 1311.627 | 3.96 | A | A |
| | Lys-Arg-Aib-Asn-Cys]- | | | | | | |
| | Tle-Thr-NH₂ | | | | | | |
| PSM-0295 | Ac-Thr-Aib-Phe-[Cys- | II | 2211.103 | 2211.238 | 4.44 | A | A |
| | Lys-Arg-Ala-Asn-Cys]- | | | | | | |
| | Tle-Thr-Asp-Ttds- | | | | | | |
| | lys(DOTA)-NH₂ | | | | | | |
| PSM-0296 | DOTA-Cmp-Thr-Aib- | I | 1791.912 | 1791.910 | 1.85 | B | C |
| | Mpa-[Cys-Lys(Me)- | | | | | | |
| | Arg(Me)-Aib-Asn-Cys]- | | | | | | |
| | Tle-Thr-NH₂. | | | | | | |
| PSM-0297 | Ac-Thr-Aib-Phe-[Cys- | I | 1277.637 | 1277.636 | 4.48 | A | A |
| | Lys-Arg-Aib-Asn-Cys]- | | | | | | |
| | Tle-Gab-NH₂ | | | | | | |
| PSM-0298 | Ac-Aib-Pcf-[Cys-Lys- | XVI | 1907.915 | 1907.915 | 4.67 | B | B |
| | Arg(Ac)-Aib-Asn-Cys]- | | | | | | |
| | Tle-Thr-Cmp- | | | | | | |
| | lys(DOTA)-NH₂ | | | | | | |
| PSM-0299 | Ac-Ala-Phe-[Cys-Lys- | II | 2096.039 | 2096.064 | 4.35 | C | B |
| | Arg-Ala-Asn-Cys]-Leu- | | | | | | |
| | Thr-Asp-Ttds- | | | | | | |
| | lys(DOTA)-NH₂ | | | | | | |
| PSM-0300 | Ac-Thr-Aib-Phe-[Cys- | VII | 1193.569 | 1193.568 | 4.82 | A | A |
| | Lys-Arg-Aib-Asn-Cys]- | | | | | | |
| | Tle-OH | | | | | | |
| PSM-0301 | Ac-Thr-Aib-Mcf-[Cys- | III | 1973.911 | 1974.031 | 4.59 | A | A |
| | Lys-Arg- | | | | | | |
| | Lys(DOTAGA-0₂0c)- | | | | | | |
| | Asn-Cys]-Tle-Thr-NH₂ | | | | | | |
| PSM-0302 | DOTA-Cmp-Thr-Aib- | I | 1840.837 | 1840.997 | 4.15 | A | A |
| | Pcf-[Cys-Lys-Arg-Amd- | | | | | | |
| | Asn-Cys]-Tle-Thr-NH₂ | | | | | | |
| PSM-0303 | Ac-Thr-Aib-Phe-[Cys- | II | 2082.049 | 2082.196 | 4.77 | B | B |
| | Lys-Gln-Aib-Asn-Cys]- | | | | | | |
| | Tle-Thr-Ttds- | | | | | | |
| | lys(DOTA)-NH₂ | | | | | | |
| PSM-0304 | DOTA-Cmp-Thr-Aib- | I | 1836.842 | 1836.997 | 4.88 | B | A |
| | 6Clw-[Cys-Lys-Cit-Aib- | | | | | | |
| | Asn-Cys]-Tle-Thr-NH₂ | | | | | | |
| PSM-0305 | DOTA-Ttds-Thr-Ala- | I | 1911.955 | 1911.967 | 4.25 | B | C |
| | Phe-[Cys-Lys-Arg-Ala- | | | | | | |
| | Asn-Cys]-Leu-Thr-NH₂ | | | | | | |
| PSM-0306 | DOTA-Cmp-Thr-Aib- | XII | 1766.873 | 1766.869 | 3.99 | A | B |
| | Pcf-[Cys-Lys-Arg-Aib- | | | | | | |
| | Asn-Cys]-Tle-en(Me)₂ | | | | | | |
| PSM-0307 | Ac-Thr-Aib-Phe-[Cys- | I | 1395.628 | 1395.655 | 4.29 | A | A |
| | Lys-Cit-Ala-Asn-Cys]- | | | | | | |
| | Tle-Thr-Asp-NH₂ | | | | | | |
| PSM-0308 | DOTA-Cmp-Thr-Aib- | I | 1863.889 | 1863.885 | 3.60 | B | C |
| | 5Clw-[Cys-Lys(Me)- | | | | | | |
| | Arg(Me)-Aib-Asn-Cys]- | | | | | | |
| | Tle-Thr-NH₂ | | | | | | |
| PSM-0310 | DOTA-Ahx-Aib-Pcf- | I | 1683.799 | 1683.794 | 4.46 | A | A |
| | [Cys-Lys-Arg-Aib-Asn- | | | | | | |
| | Cys]-Tle-Thr-NH₂ | | | | | | |
| PSM-0311 | GaDOTA-Cmp-Thr- | XXc | 1789.733 | 1789.729 | 4.01 | A | A |
| | Aib-Pcf-[Cys-Lys(Me)- | | | | | | |
| | Arg(Me)-Aib-Asn-Cys]- | | | | | | |
| | Tle-NH₂ | | | | | | |
| PSM-0312 | Hex-Thr-Aib-Phe-[Cys- | XXb | 2453.098 | 2453.092 | 5.95 | A | A |
| | Lys-Arg-Aib-Asn-Cys]- | | | | | | |
| | Tle-Thr-Asp-Ttds- | | | | | | |
| | lys(LuDOTA)-NH₂ | | | | | | |
| PSM-0313 | Ac-Thr-Ala-Phe-[Cys- | VII | 1351.638 | 1351.697 | 4.54 | B | C |
| | Lys-Arg-Ala-Asn-Cys]- | | | | | | |
| | Nle-Thr-Gab-OH | | | | | | |
| PSM-0314 | DOTA-Cmp-Thr-Aib- | I | 1812.842 | 1812.851 | 4.23 | A | A |
| | Pcf-[Cys-Lys-Arg-ams- | | | | | | |
| | Asn-Cys]-Tle-Thr-NH₂ | | | | | | |
| PSM-0315 | Ac-Thr-Aib-Phe-[Cys- | I | 1394.644 | 1394.697 | 4.09 | A | B |
| | Lys-arg-Ala-Asn-Cys]- | | | | | | |
| | Tle-Thr-Asp-NH₂ | | | | | | |
| PSM-0316 | DOTA-Pab-Aib-Phe- | I | 1655.791 | 1655.790 | 4.37 | A | A |
| | [Cys-Lys-Arg-Aib-Asn- | | | | | | |
| | Cys]-Tle-Thr-NH₂ | | | | | | |
| PSM-0317 | DOTA-Cmp-Thr-Aib- | I | 1810.863 | 1810.862 | 4.10 | A | A |
| | Pcf-[Cys-Lys-Har-Aib- | | | | | | |
| | Asn-Cys]-Tle-Thr-NH₂ | | | | | | |
| PSM-0318 | Ac-Thr-Aib-Pcf-[Cys- | I | 1442.620 | 1442.690 | 4.88 | A | A |
| | Lys-Arg-Aib-Asn-Cys]- | | | | | | |
| | Tle-Thr-Asp-NH₂ | | | | | | |
| PSM-0319 | Ac-Thr-Ala-Nmf-[Cys- | I | 1293.632 | 1293.634 | 4.21 | A | B |
| | Lys-Arg-Aib-Asn-Cys]- | | | | | | |
| | Tle-Thr-NH₂ | | | | | | |
| PSM-0320 | Ac-Aib-Pcf-[Cys-Lys- | VI | 1568.736 | 1568.737 | 4.19 | A | A |
| | Arg(Me)-Aib-Asn-Cys]- | | | | | | |
| | Tle-en-DOTA | | | | | | |
| PSM-0321 | Ac-Thr-Aib-Phe-[Cys- | II | 1720.839 | 1720.803 | 3.82 | A | A |
| | Lys-Arg-Apc(DOTA)- | | | | | | |
| | Asn-Cys]-Tle-Thr-NH₂ | | | | | | |
| PSM-0322 | Ac-Thr-Aib-Pcf-[Cys- | III | 1967.936 | 1967.952 | 4.90 | A | B |
| | Lys-Cit-Aib-Asn-Cys]- | | | | | | |
| | Tle-Thr-lys(DOTA- | | | | | | |
| | Cmp)-NH₂ | | | | | | |
| PSM-0323 | Ac-Phe-[Cys-Lys-Arg- | II | 2025.002 | 2024.998 | 4.36 | D | D |
| | Ala-Asn-Cys]-Leu-Thr- | | | | | | |
| | Asp-Ttds-lys(DOTA)- | | | | | | |
| | NH₂ | | | | | | |
| PSM-0324 | HPA-Aib-Pcf-[Cys-Lys- | II | 1825.873 | 1825.869 | 4.15 | A | A |
| | Arg-Lys(DOTA-APAc)- | | | | | | |
| | Asn-Cys]-Tle-Thr-NH₂ | | | | | | |
| PSM-0325 | LuDOTA-Cmp-Thr- | XXb | 1969.748 | 1969.765 | 5.12 | A | A |
| | Aib-Pcf-[Cys-Lys-Cit- | | | | | | |
| | Aib-Asn-Cys]-Tle-Thr- | | | | | | |
| | NH₂ | | | | | | |
| PSM-0326 | Ac-Thr-Aib-Phe-[Cys- | II | 1722.855 | 1722.943 | 4.02 | A | B |
| | Lys-Arg-Lys(DOTA)- | | | | | | |
| | Asn-Cys]-Tle-Thr-NH₂ | | | | | | |
| PSM-0327 | Ac-Thr-Aib-Phe-[cys- | I | 1293.632 | 1293.704 | 4.13 | D | D |
| | Lys-Arg-Aib-Asn-cys]- | | | | | | |
| | Tle-Thr-NH₂ | | | | | | |
| PSM-0328 | SaPr-Aib-Pcf-[Cys-Lys- | II | 1833.809 | 1833.808 | 4.55 | A | A |
| | Arg-Aib-Asn-Cys]-Tle- | | | | | | |
| | Thr-lys(DOTA)-NH₂ | | | | | | |
| PSM-0329 | Ac-Thr-Aib-Pcf-[Cys- | II | 1967.936 | 1968.064 | 4.70 | A | B |
| | Lys-Cit-Aib-Asn-Cys]- | | | | | | |
| | Tle-Thr-Cmp- | | | | | | |
| | lys(DOTA)-NH₂ | | | | | | |
| PSM-0330 | Ac-Thr-Ala-Phe-[Cys- | II | 2197.087 | 2197.118 | 4.42 | B | B |
| | Lys-Arg-Ala-Asn-Cys]- | | | | | | |
| | Leu-Thr-Asp-Ttds- | | | | | | |
| | lys(DOTA)-NH₂ | | | | | | |
| PSM-0331 | Ac-Thr-Aib-Phe-[Cys- | I | 1293.632 | 1293.691 | 4.44 | B | D |
| | Lys-Arg-Aib-Asn-cys]- | | | | | | |
| | Tle-Thr-NH₂ | | | | | | |
| PSM-0332 | DOTA-Cmp-Thr-Aib- | XIV | 1709.815 | 1709.811 | 3.57 | A | A |
| | Pcf-[Smc-Lys-Arg-Aib- | | | | | | |
| | Asn-Cys]-Tle-NH₂ | | | | | | |
| | (alternative: DOTA- | | | | | | |
| | Cmp-Thr-Aib-Pcf-[Cys- | | | | | | |
| | Lys-Arg-Aib-Asn-Smc]- | | | | | | |
| | Tle-NH₂) | | | | | | |
| PSM-0333 | DOTA-Cmp-Thr-Aib- | I | 1814.881 | 1814.874 | 3.66 | D | D |
| | ₂Qi-[Cys-Lys-Cit-Aib- | | | | | | |
| | Asn-Cys]-Tle-Thr-NH₂ | | | | | | |
| PSM-0334 | Ac-Thr-Aib-Phe-[Cys- | I | 1380.617 | 1380.622 | 4.46 | A | B |
| | Lys-Gln-Aib-Asn-Cys]- | | | | | | |
| | Tle-Thr-Asp-NH₂ | | | | | | |
| PSM-0335 | Ac-Thr-Ala-Phe-[Cys- | I | 1337.622 | 1337.682 | 4.42 | C | B |
| | Lys-Arg-Ala-Ala-Cys]- | | | | | | |
| | Nle-Thr-Asp-NH₂ | | | | | | |
| PSM-0336 | Ac-Thr-Aib-Pcf-[Cys- | I | 1327.593 | 1327.695 | 4.72 | A | A |
| | Lys-Arg-Aib-Asn-Cys]- | | | | | | |
| | Tle-Thr-NH₂ | | | | | | |
| PSM-0337 | DOTA-Cmp-Thr-Aib- | I | 1841.928 | 1842.004 | 0.00 | C | D |
| | ₂Qi-[Cys-Lys(Me)- | | | | | | |
| | Arg(Me)-Aib-Asn-Cys]- | | | | | | |
| | Tle-Thr-NH₂ | | | | | | |
| PSM-0338 | Ac-Thr-Aib-Pcf-[Cys- | I | 1284.576 | 1284.674 | 5.66 | A | A |
| | Lys-Nle-Aib-Asn-Cys]- | | | | | | |
| | Tle-Thr-NH₂ | | | | | | |
| PSM-0339 | Ac-Thr-Aib-Phe-[Cys- | II | 2005.013 | 2005.110 | 4.19 | A | A |
| | Lys-Arg-Aib-Asn-Cys]- | | | | | | |
| | Tle-Thr-Cmp- | | | | | | |
| | lys(DOTAGA)-NH₂ | | | | | | |
| PSM-0340 | DOTA-Cmp-Thr-Aib- | I | 1810.863 | 1810.862 | 4.46 | B | A |
| | Pcf-[Cys-Lys-Nmr-Aib- | | | | | | |
| | Asn-Cys]-Tle-Thr-NH₂ | | | | | | |
| PSM-0341 | DOTA-Cmp-Thr-Aib- | I | 1810.851 | 1810.855 | 4.43 | B | A |
| | Pcf-[Cys-Lys-Lys(Ac)- | | | | | | |
| | Aib-Asn-Cys]-Tle-Thr- | | | | | | |
| | NH₂ | | | | | | |
| PSM-0342 | DOTA-Ttds-Thr-Ala- | I | 2026.982 | 2026.981 | 4.00 | A | B |
| | Phe-[Cys-Lys-Arg-Ala- | | | | | | |
| | Asn-Cys]-Tle-Thr-Asp- | | | | | | |
| | NH₂ | | | | | | |
| PSM-0343 | DOTA-Cmp-Thr-glu- | I | 1840.837 | 1840.999 | 3.84 | D | D |
| | Pcf-[Cys-Lys-Arg-Aib- | | | | | | |
| | Asn-Cys]-Tle-Thr-NH₂ | | | | | | |
| PSM-0345 | DOTA-Cmp-Thr-Aib- | I | 1709.815 | 1709.818 | 3.86 | A | A |
| | Pcf-[Cys-Lys-Arg(Me)- | | | | | | |
| | Aib-Asn-Cys]-Tle-NH₂ | | | | | | |
| PSM-0346 | DOTA-Cmp-Thr-Amd- | I | 1840.837 | 1841.000 | 3.94 | B | C |
| | Pcf-[Cys-Lys-Arg-Aib- | | | | | | |
| | Asn-Cys]-Tle-Thr-NH₂ | | | | | | |
| PSM-0347 | Ac-Thr-Aib-Phe-[Cys- | XXb | 2104.909 | 2104.923 | 4.48 | A | A |
| | Lys-Arg-Aib-Asn-Cys]- | | | | | | |
| | Tle-Thr-Cmp- | | | | | | |
| | lys(LuDOTA)-NH₂ | | | | | | |
| PSM-0348 | Ac-Thr-Aib-Phe-[cys- | I | 1293.632 | 1293.705 | 4.01 | D | D |
| | Lys-Arg-Aib-Asn-Cys]- | | | | | | |
| | Tle-Thr-NH₂ | | | | | | |
| PSM-0349 | Ac-Aib-Pcf-[Cys-Lys- | II | 1865.905 | 1865.894 | 4.67 | A | A |
| | Arg-Aib-Asn-Cys]-Tle- | | | | | | |
| | Thr-Cmp-lys(DOTA)- | | | | | | |
| | NH₂ | | | | | | |
| PSM-0350 | Ac-Thr-Aib-Phe-[Cys- | I | 1293.632 | 1293.634 | 4.97 | B | C |
| | Lys-Arg-Aib-Asn-Cys]- | | | | | | |
| | leu-Thr-NH₂ | | | | | | |
| PSM-0351 | DOTA-Cmp-Thr-Aib- | I | 1810.863 | 1810.878 | 4.24 | A | A |
| | Pcf-[Cys-Lys-Arg(Me)- | | | | | | |
| | Aib-Asn-Cys]-Tle-Thr- | | | | | | |
| | NH₂ | | | | | | |
| PSM-0352 | DOTA-Bal-Aib-Pcf- | I | 1641.752 | 1641.747 | 4.25 | A | A |
| | [Cys-Lys-Arg-Aib-Asn- | | | | | | |
| | Cys]-Tle-Thr-NH₂ | | | | | | |
| PSM-0353 | Iva-Aib-Pcf-[Cys-Lys- | II | 1837.910 | 1838.003 | 5.21 | A | A |
| | Arg-Lys(DOTA-APAc)- | | | | | | |
| | Asn-Cys]-Tle-Thr-NH₂ | | | | | | |
| PSM-0354 | DOTA-Cmp-Thr-Aib- | I | 1723.831 | 1723.838 | 3.98 | A | B |
| | Mcf-[Cys-Lys(Me)- | | | | | | |
| | Arg(Me)-Aib-Asn-Cys]- | | | | | | |
| | Tle-NH₂ | | | | | | |
| PSM-0355 | DOTA-Cmp-Thr-Aib- | XXI | 1852.910 | 1852.945 | 3.51 | B | A |
| | Pcf-[Cys-Lys(Me)- | | | | | | |
| | RMe3-Aib-Asn-Cys]- | | | | | | |
| | Tle-Thr-NH₂ | | | | | | |
| PSM-0356 | GaDOTA-Cmp-Thr- | XXc | 1890.780 | 1890.777 | 3.89 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0357 | DOTA-Cmp-Ile-Aib- | I | 1774.922 | 1774.922 | 5.09 | B | B |
| | Phe-[Cys-Lys-Arg-Aib- | | | | | | |
| | Asn-Cys]-Tle-Thr-NH₂ | | | | | | |
| PSM-0358 | Ac-Thr-Aib-Phe-[Cys- | XXa | 2249.946 | 2250.041 | 4.55 | A | A |
| | Lys-Arg-Lys(InDOTA- | | | | | | |
| | Ttds)-Asn-Cys]-Tle- | | | | | | |
| | Thr-Asp-NH₂ | | | | | | |
| PSM-0359 | DOTA-Cmp-Thr-Aib- | I | 1880.791 | 1880.783 | 4.95 | D | D |
| | 5Brw-[Cys-Lys-Cit- | | | | | | |
| | Aib-Asn-Cys]-Tle-Thr- | | | | | | |
| | NH₂ | | | | | | |
| PSM-0361 | DOTA-Cmp-Thr-Aib- | I | 1661.838 | 1661.835 | 4.00 | A | A |
| | Phe-[Cys-Lys-Arg-Aib- | | | | | | |
| | Asn-Cys]-Tle-NH₂ | | | | | | |
| PSM-0362 | InDOTA-Cmp-Thr-Aib- | Xxa | 1906.728 | 1906.727 | 5.29 | A | A |
| | Pcf-[Cys-Lys-Arg-Aib- | | | | | | |
| | Asn-Cys]-Tle-Thr-NH₂ | | | | | | |
| PSM-0363 | Ac-Thr-Aib-Phe-[Cys- | I | 1367.585 | 1367.625 | 4.43 | B | A |
| | Lys-Glu-Ala-Asn-Cys]- | | | | | | |
| | Tle-Thr-Asp-NH₂ | | | | | | |
| PSM-0365 | DOTA-Cmp-Thr-Aib- | I | 1797.831 | 1797.894 | 4.56 | A | A |
| | Pcf-[Cys-Lys-Cit-Aib- | | | | | | |
| | Asn-Cys]-Tle-Thr-NH₂ | | | | | | |
| PSM-0366 | DOTA-Cmp-Thr-amd- | I | 1840.837 | 1840.836 | 4.05 | B | C |
| | Pcf-[Cys-Lys-Arg-Aib- | | | | | | |
| | Asn-Cys]-Tle-Thr-NH₂ | | | | | | |
| PSM-0367 | Ac-Thr-Aib-Pcf-[Cys- | I | 1226.546 | 1226.543 | 5.15 | A | A |
| | Lys-Arg-Aib-Asn-Cys]- | | | | | | |
| | Tle-NH₂ | | | | | | |
| PSM-0368 | DOTA-Ahx-Thr-Aib- | I | 1750.886 | 1750.973 | 4.06 | A | A |
| | Phe-[Cys-Lys-Arg-Aib- | | | | | | |
| | Asn-Cys]-Tle-Thr-NH₂ | | | | | | |
| PSM-0369 | DOTA-Cmp-Thr-Ams- | I | 1812.842 | 1812.874 | 4.03 | A | A |
| | Pcf-[Cys-Lys-Arg-Aib- | | | | | | |
| | Asn-Cys]-Tle-Thr-NH₂ | | | | | | |
| PSM-0370 | Ac-Thr-Aib-Pcf-[Cys- | III | 1901.890 | 1902.012 | 4.39 | A | A |
| | Lys-Arg-Lys(DOTA- | | | | | | |
| | O₂0c)-Asn-Cys]-Tle- | | | | | | |
| | Thr-NH₂ | | | | | | |
| PSM-0371 | nBuCAyl-Thr-Aib-Pcf- | IV | 2024.010 | 2024.003 | 5.94 | A | A |
| | [Cys-Lys-Arg-Aib-Asn- | | | | | | |
| | Cys]-Tle-Thr-Cmp- | | | | | | |
| | lys(DOTA)-NH₂ | | | | | | |
| PSM-0372 | Ac-Thr-Aib-Pmf-[Cys- | I | 1307.648 | 1307.645 | 4.12 | A | A |
| | Lys-Arg-Aib-Asn-Cys]- | | | | | | |
| | Tle-Thr-NH₂ | | | | | | |
| PSM-0373 | LuDOTA-Cmp-Thr- | XXb | 1996.796 | 1996.792 | 4.46 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0374 | Ac-Thr-Aib-Phe-[Cys- | I | 1408.659 | 1408.683 | 4.32 | A | A |
| | Lys-Arg-Aib-Asn-Cys]- | | | | | | |
| | Tle-Thr-Asp-NH₂ | | | | | | |
| PSM-0375 | Ac-Thr-Aib-Phe-[Cys- | I | 1321.664 | 1321.639 | 4.53 | A | A |
| | Lys-Arg-Deg-Asn-Cys]- | | | | | | |
| | Tle-Thr-NH₂ | | | | | | |
| PSM-0376 | Ac-Thr-Aib-Phe-[Cys- | I | 1378.664 | 1378.776 | 5.33 | A | A |
| | Lys-Arg-Aib-Asn-Cys]- | | | | | | |
| | Tle-Trp-NH₂ | | | | | | |
| PSM-0377 | DOTA-Cmp-Thr-Aib- | I | 1796.847 | 1796.839 | 4.16 | A | A |
| | Pcf-[Cys-Lys-Arg-Aib- | | | | | | |
| | Asn-Cys]-Val-Nmt-NH₂ | | | | | | |
| PSM-0378 | H-Cmp-Thr-Aib-Phe- | II | 2016.065 | 2016.057 | 3.84 | A | A |
| | [Cys-Lys-Arg-Aib-Asn- | | | | | | |
| | Cys]-Tle-Thr-Cmp- | | | | | | |
| | lys(DOTA)-NH₂ | | | | | | |
| PSM-0379 | Ac-Cmp-Thr-Aib-Pcf- | VI | 1780.852 | 1780.837 | 4.51 | A | A |
| | [Cys-Lys-Arg-Aib-Asn- | | | | | | |
| | Cys]-Tle-en-DOTA | | | | | | |
| PSM-0380 | DOTA-Cmp-Thr-Aib- | I | 1723.831 | 1723.828 | 3.93 | A | A |
| | Pcf-[Cys-Lys(Me)- | | | | | | |
| | Arg(Me)-Aib-Asn-Cys]- | | | | | | |
| | Tle-NH₂ | | | | | | |
| PSM-0381 | DOTA-Pamb-Aib-Pcf- | I | 1602.720 | 1602.722 | 4.81 | A | A |
| | [Cys-Lys-Arg-Aib-Asn- | | | | | | |
| | Cys]-Tle-NH₂ | | | | | | |
| PSM-0382 | DOTA-Cmp-Thr-Aib- | I | 1812.842 | 1812.886 | 4.06 | A | A |
| | Pcf-[Cys-Lys-Arg-Ams- | | | | | | |
| | Asn-Cys]-Tle-Thr-NH₂ | | | | | | |
| PSM-0383 | Ac-Thr-Aib-Mcf-[Cys- | I | 1442.620 | 1442.688 | 4.86 | A | A |
| | Lys-Arg-Aib-Asn-Cys]- | | | | | | |
| | Tle-Thr-Asp-NH₂ | | | | | | |
| PSM-0384 | DOTA-Cmp-Thr-Aib- | V | 1738.841 | 1738.843 | 4.03 | A | A |
| | Pcf-[Cys-Lys-Arg-Aib- | | | | | | |
| | Asn-Cys]-Tle-en | | | | | | |
| PSM-0385 | Ac-Thr-Aib-Phe-[Cys- | II | 1694.812 | 1694.943 | 4.07 | B | C |
| | Lys-Gln-Lys(DOTA)- | | | | | | |
| | Asn-Cys]-Tle-Thr-NH₂ | | | | | | |
| PSM-0386 | DOTA-Cmp-Thr-Aib- | I | 1842.843 | 1842.834 | 3.40 | D | D |
| | Fso-[Cys-Lys-Arg-Aib- | | | | | | |
| | Asn-Cys]-Tle-Thr-NH₂ | | | | | | |
| PSM-0387 | Ac-Thr-Aib-Miy-[Cys- | I | 1463.555 | 1463.552 | 3.37 | D | D |
| | Lys(Me)-Arg(Me)-Aib- | | | | | | |
| | Asn-Cys]-Tle-Thr-NH₂ | | | | | | |
| PSM-0388 | Ac-Thr-Aib-Phe-[Cys- | I | 1289.637 | 1289.639 | 4.64 | A | A |
| | Lys-Arg-Aib-Asn-Cys]- | | | | | | |
| | Tle-Pro-NH₂ | | | | | | |
| PSM-0389 | Ac-Thr-Ala-Phe-[Cys- | I | 1380.628 | 1380.681 | 4.26 | A | B |
| | Lys-Arg-Ala-Asn-Cys]- | | | | | | |
| | Nle-Thr-asp-NH₂ | | | | | | |
| PSM-0390 | DOTA-Cmp-Thr-Aib- | I | 1762.841 | 1762.835 | 4.16 | A | A |
| | 6Clw-[Cys-Lys(Me)- | | | | | | |
| | Arg(Me)-Aib-Asn-Cys]- | | | | | | |
| | Tle-NH₂ | | | | | | |
| PSM-0391 | Ac-Thr-Aib-Phe-[Cys- | VI | 1764.902 | 1764.986 | 4.32 | A | A |
| | Lys-Arg-Aib-Asn-Cys]- | | | | | | |
| | Tle-Thr-Ape-DOTA | | | | | | |
| PSM-0392 | Ac-Thr-Aib-Phe-[Cys- | I | 1438.650 | 1438.676 | 4.14 | A | A |
| | Lys-Arg-4Tfp-Asn- | | | | | | |
| | Cys]-Tle-Thr-Asp-NH₂ | | | | | | |
| PSM-0393 | Ac-Thr-Aib-Pcf-[Cys- | V | 1269.588 | 1269.585 | 4.64 | A | A |
| | Lys-Arg-Aib-Asn-Cys]- | | | | | | |
| | Tle-en | | | | | | |
| PSM-0394 | Ac-Thr-Aib-Phe-[Cys- | I | 1265.601 | 1265.600 | 4.09 | B | B |
| | Lys-Arg-Aib-Asn-Cys]- | | | | | | |
| | Aib-Thr-NH₂ | | | | | | |
| PSM-0395 | DOTA-Cmp-Thr-Aib- | I | 1707.860 | 1707.864 | 3.73 | A | A |
| | Pff-[Cys-Lys(Me)- | | | | | | |
| | Arg(Me)-Aib-Asn-Cys]- | | | | | | |
| | Tle-NH₂ | | | | | | |
| PSM-0396 | DOTA-Cmp-Thr-Aib- | I | 1757.857 | 1757.849 | 4.21 | A | A |
| | Mtf-[Cys-Lys(Me)- | | | | | | |
| | Arg(Me)-Aib-Asn-Cys]- | | | | | | |
| | Tle-NH₂ | | | | | | |
| PSM-0397 | Ac-Thr-Aib-Phe-[Cys- | II | 1837.882 | 1838.006 | 3.88 | A | B |
| | Lys-Arg-Lys(DOTA)- | | | | | | |
| | Asn-Cys]-Tle-Thr-Asp- | | | | | | |
| | NH₂ | | | | | | |
| PSM-0398 | DOTA-Ttds-Thr-Aib- | I | 2055.013 | 2055.066 | 4.23 | A | A |
| | Phe-[Cys-Lys-Arg-Aib- | | | | | | |
| | Asn-Cys]-Tle-Thr-Asp- | | | | | | |
| | NH₂ | | | | | | |
| PSM-0399 | InDOTA-Cmp-Thr-Aib- | XXa | 1907.712 | 1907.724 | 5.23 | A | A |
| | Pcf-[Cys-Lys-Cit-Aib- | | | | | | |
| | Asn-Cys]-Tle-Thr-NH₂ | | | | | | |
| PSM-0400 | Hex-Thr-Aib-Pcf-[Cys- | III | 2023.015 | 2023.036 | 5.90 | A | A |
| | Lys-Arg-Aib-Asn-Cys]- | | | | | | |
| | Tle-Thr-lys(DOTA- | | | | | | |
| | Cmp)-NH₂ | | | | | | |
| PSM-0401 | Ac-Thr-Aib-Phe-[Cys- | III | 1867.928 | 1867.955 | 4.11 | A | A |
| | Lys-Arg-Lys(DOTA- | | | | | | |
| | 0₂0c)-Asn-Cys]-Tle- | | | | | | |
| | Thr-NH₂ | | | | | | |
| PSM-0402 | Ac-Thr-Aib-Phe-[Cys- | I | 1293.632 | 1293.740 | 4.14 | A | A |
| | Lys-Arg-Aib-asn-Cys]- | | | | | | |
| | Tle-Thr-NH₂ | | | | | | |
| PSM-0403 | Ac-Thr-Aib-Phe-[Cys- | I | 1474.706 | 1474.731 | 4.57 | A | A |
| | Lys-Arg-Oic-Asn-Cys]- | | | | | | |
| | Tle-Thr-Asp-NH₂ | | | | | | |
| PSM-0404 | Ac-Aib-Pcf-[Cys-Lys- | II | 1740.821 | 1740.822 | 4.63 | A | A |
| | Arg-Aib-Asn-Cys]-Tle- | | | | | | |
| | Thr-lys(DOTA)-NH₂ | | | | | | |
| PSM-0405 | Hex-Thr-Ala-Phe-[Cys- | II | 2253.150 | 2253.159 | 5.63 | A | B |
| | Lys-Arg-Ala-Asn-Cys]- | | | | | | |
| | Leu-Thr-Asp-Ttds- | | | | | | |
| | lys(DOTA)-NH₂ | | | | | | |
| PSM-0406 | DOTA-Cmp-Thr-Aib- | I | 1836.842 | 1836.997 | 4.87 | C | D |
| | 5Clw-[Cys-Lys-Cit-Aib- | | | | | | |
| | Asn-Cys]-Tle-Thr-NH₂ | | | | | | |
| PSM-0407 | DOTA-Cmp-Thr-Ams- | I | 1828.837 | 1828.837 | 3.89 | A | A |
| | Pcf-[Cys-Lys-Arg-Ams- | | | | | | |
| | Asn-Cys]-Tle-Thr-NH₂ | | | | | | |
| PSM-0408 | Ac-Thr-Nmg-Phe-[Cys- | I | 1279.617 | 1279.687 | 3.84 | B | C |
| | Lys-Arg-Aib-Asn-Cys]- | | | | | | |
| | Tle-Thr-NH₂ | | | | | | |
| PSM-0409 | DOTA-Cmp-Thr-Aib- | I | 1690.865 | 1690.863 | 2.59 | B | C |
| | Opa-[Cys-Lys(Me)- | | | | | | |
| | Arg(Me)-Aib-Asn-Cys]- | | | | | | |
| | Tle-NH₂ | | | | | | |
| PSM-0410 | DOTA-Cmp-Thr-Aib- | I | 1810.863 | 1810.874 | 4.29 | A | A |
| | Pcf-[Cys-Lys(Me)-Arg- | | | | | | |
| | Aib-Asn-Cys]-Tle-Thr- | | | | | | |
| | NH₂ | | | | | | |
| PSM-0411 | Ac-Thr-Aib-Hfe-[Cys- | I | 1307.648 | 1307.646 | 4.03 | A | A |
| | Lys-Arg-Aib-Asn-Cys]- | | | | | | |
| | Tle-Thr-NH₂ | | | | | | |
| PSM-0412 | Ac-Thr-Aib-Mmf-[Cys- | I | 1307.648 | 1307.653 | 4.19 | A | A |
| | Lys-Arg-Aib-Asn-Cys]- | | | | | | |
| | Tle-Thr-NH₂ | | | | | | |
| PSM-0413 | DOTA-PPAc-Aib-Phe- | I | 1662.833 | 1662.902 | 3.54 | A | B |
| | [Cys-Lys-Arg-Aib-Asn- | | | | | | |
| | Cys]-Tle-Thr-NH₂ | | | | | | |
| PSM-0414 | Ac-Thr-Ala-Amf-[Cys- | I | 1293.632 | 1293.636 | 4.34 | D | B |
| | Lys-Arg-Aib-Asn-Cys]- | | | | | | |
| | Tle-Thr-NH₂ | | | | | | |
| PSM-0415 | Ac-Thr-Aib-Phe-[Cys- | III | 1847.939 | 1848.045 | 4.11 | A | B |
| | Lys-Arg-Lys(DOTA- | | | | | | |
| | Cmp)-Asn-Cys]-Tle- | | | | | | |
| | Thr-NH₂ | | | | | | |
| PSM-0416 | DOTA-Cmp-Thr-Aib- | I | 1840.837 | 1841.002 | 3.93 | A | A |
| | Pcf-[Cys-Lys-Arg-Glu- | | | | | | |
| | Asn-Cys]-Tle-Thr-NH₂ | | | | | | |
| PSM-0418 | LuDOTA-Ttds-Thr-Aib- | XXb | 2097.888 | 2097.882 | 4.34 | A | A |
| | Phe-[Cys-Lys-Arg-Ala- | | | | | | |
| | Asn-Cys]-Tle-Thr-NH₂ | | | | | | |
| PSM-0419 | Ac-Thr-Aib-Phe-[Cys- | III | 1855.907 | 1856.014 | 4.23 | A | A |
| | Lys-Arg-Lys(DOTA- | | | | | | |
| | Pamb)-Asn-Cys]-Tle- | | | | | | |
| | Thr-NH₂ | | | | | | |
| PSM-0420 | DOTA-Cmp-Thr-Aib- | XIV | 1737.846 | 1737.842 | 3.56 | A | A |
| | Pcf-[Smc-Lys(Me)- | | | | | | |
| | Arg(Me)-Aib-Asn-Cys]- | | | | | | |
| | Tle-NH₂ (alternative: | | | | | | |
| | DOTA-Cmp-Thr-Aib- | | | | | | |
| | Pcf-[Cys-Lys(Me)- | | | | | | |
| | Arg(Me)-Aib-Asn- | | | | | | |
| | Smc]-Tle-NH₂) | | | | | | |
| PSM-0421 | Ac-Thr-Aib-Pcf-[Cys- | I | 1328.577 | 1328.681 | 5.41 | B | C |
| | Lys-Cit-Aib-Asn-Cys]- | | | | | | |
| | leu-Thr-NH₂ | | | | | | |
| PSM-0422 | Ac-Thr-Aib-Phe-[Cys- | I | 1378.612 | 1378.638 | 4.39 | A | C |
| | Tap-Arg-Ala-Asn-Cys]- | | | | | | |
| | Tle-Thr-Asp-NH₂ | | | | | | |
| PSM-0423 | Ac-Thr-Aib-Phe-[Cys- | III | 1996.996 | 1997.160 | 4.41 | B | B |
| | Lys-Gln-Lys(DOTA- | | | | | | |
| | Ttds)-Asn-Cys]-Tle- | | | | | | |
| | Thr-NH₂ | | | | | | |
| PSM-0424 | Ac-Aib-Pcf-[Cys-Lys- | I | 1125.498 | 1125.497 | 3.94 | A | A |
| | Arg-Aib-Asn-Cys]-Tle- | | | | | | |
| | NH₂ | | | | | | |
| PSM-0425 | Hex-Thr-Aib-Phe-[Cys- | II | 2281.181 | 2281.175 | 5.63 | A | A |
| | Lys-Arg-Aib-Asn-Cys]- | | | | | | |
| | Tle-Thr-Asp-Ttds- | | | | | | |
| | lys(DOTA)-NH₂ | | | | | | |
| PSM-0426 | Ac-Thr-Aib-Phe-[Cys- | I | 1263.622 | 1263.620 | 4.36 | A | A |
| | Lys-Arg-Aib-Asn-Cys]- | | | | | | |
| | Tle-Bal-NH₂ | | | | | | |
| PSM-0427 | nBuCAyl-Thr-Aib-Pcf- | IV | 2059.015 | 2059.012 | 5.99 | A | B |
| | [Cys-Lys-Opy-Aib-Asn- | | | | | | |
| | Cys]-Tle-Thr-Cmp- | | | | | | |
| | lys(DOTA)-NH₂ | | | | | | |
| PSM-0428 | Ac-Thr-Aib-Phe-[Cys- | II | 1932.991 | 1933.101 | 4.23 | A | A |
| | Lys-Arg-Aib-Asn-Cys]- | | | | | | |
| | Tle-Thr-Cmp- | | | | | | |
| | lys(DOTA)-NH₂ | | | | | | |
| PSM-0430 | AF488Ahx-Ttds-Ttds- | XIII | 2849.177 | 2849.162 | 6.03 | Nd | Nd |
| | Thr-Aib-Pcf-[Cys- | | | | | | |
| | Lys(Me)-Arg(Me)-Aib- | | | | | | |
| | Asn-Cys]-Tle-Thr-NH₂ | | | | | | |
| PSM-0431 | DOTA-Cmp-Thr-Aib- | I | 1797.831 | 1797.899 | 5.07 | B | A |
| | Mcf-[Cys-Lys-Cit-Aib- | | | | | | |
| | Asn-Cys]-Tle-Thr-NH₂ | | | | | | |
| PSM-0432 | DOTA-Pamb-Aib-Pcf- | I | 1673.757 | 1673.754 | 4.56 | A | A |
| | [Cys-Lys-Arg-Aib-Asn- | | | | | | |
| | Cys]-Tle-Bal-NH₂ | | | | | | |
| PSM-0433 | DOTA-Cmp-Thr-Aib- | I | 1695.799 | 1695.800 | 4.52 | A | A |
| | Pcf-[Cys-Lys-Arg-Aib- | | | | | | |
| | Asn-Cys]-Tle-NH₂ | | | | | | |
| PSM-0434 | nBuCAyl-Thr-Aib-Phe- | IV | 1991.033 | 1991.030 | 5.57 | B | B |
| | [Cys-Lys-Cit-Aib-Asn- | | | | | | |
| | Cys]-Tle-Thr-Cmp- | | | | | | |
| | lys(DOTA)-NH₂ | | | | | | |
| PSM-0435 | Ac-Thr-Aib-Phe-[Cys- | I | 1394.607 | 1394.641 | 4.41 | A | B |
| | Gln-Arg-Ala-Asn-Cys]- | | | | | | |
| | Tle-Thr-Asp-NH₂ | | | | | | |
| PSM-0436 | Ac-Thr-Ala-phe-[Cys- | I | 1380.628 | 1380.689 | 4.36 | D | D |
| | Lys-Arg-Ala-Asn-Cys]- | | | | | | |
| | Nle-Thr-Asp-NH₂ | | | | | | |
| PSM-0437 | DOTA-Bal-Aib-Phe- | I | 1607.791 | 1607.793 | 3.84 | A | B |
| | [Cys-Lys-Arg-Aib-Asn- | | | | | | |
| | Cys]-Tle-Thr-NH₂ | | | | | | |
| PSM-0438 | Ac-Thr-Aib-Phe-[Cys- | XXb | 2039.846 | 2039.848 | 4.44 | A | A |
| | Lys-Arg-Lys(LuDOTA- | | | | | | |
| | O₂0c)-Asn-Cys]-Tle- | | | | | | |
| | Thr-NH₂ | | | | | | |
| PSM-0439 | Ac-Thr-Aib-Phe-[Cys- | I | 1428.628 | 1428.651 | 4.23 | A | C |
| | Aph-Arg-Ala-Asn-Cys]- | | | | | | |
| | Tle-Thr-Asp-NH₂ | | | | | | |
| PSM-0440 | Ac-Thr-Aib-Phe-[Cys- | XXa | 2042.872 | 2042.886 | 4.41 | A | A |
| | Lys-Arg-Aib-Asn-Cys]- | | | | | | |
| | Tle-Thr-Cmp- | | | | | | |
| | lys(InDOTA)-NH₂ | | | | | | |
| PSM-0441 | Ac-Thr-Aib-Cys(Bzl)- | I | 1339.620 | 1339.624 | 4.29 | A | B |
| | [Cys-Lys-Arg-Aib-Asn- | | | | | | |
| | Cys]-Tle-Thr-NH₂ | | | | | | |
| PSM-0442 | Ac-Aib-Phe-[Cys-Lys- | I | 1307.612 | 1307.618 | 4.33 | A | A |
| | Arg-Aib-Asn-Cys]-Tle- | | | | | | |
| | Thr-Asp-NH₂ | | | | | | |
| PSM-0443 | Ac-Aib-Pcf-[Cys-Lys- | I | 1012.414 | 1012.408 | 2.95 | B | D |
| | Arg-Aib-Asn-Cys]-NH₂ | | | | | | |
| PSM-0444 | Iva-Aib-Pcf-[Cys-Lys- | II | 1782.868 | 1782.863 | 5.58 | A | A |
| | Arg-Aib-Asn-Cys]-Tle- | | | | | | |
| | Thr-lys(DOTA)-NH₂ | | | | | | |
| PSM-0445 | DOTA-Cmp-Thr-Aib- | I | 1796.847 | 1796.905 | 4.41 | A | A |
| | Pcf-[Cys-Lys-Arg-Aib- | | | | | | |
| | Asn-Cys]-Tle-Thr-NH₂ | | | | | | |
| PSM-0447 | DOTA-Aib-Phe-[Cys- | I | 1536.754 | 1536.817 | 3.45 | D | D |
| | Lys-Arg-Aib-Asn-Cys]- | | | | | | |
| | Tle-Thr-NH₂ | | | | | | |
| PSM-0448 | DOTA-Pab-Thr-Aib- | I | 1756.839 | 1756.907 | 3.94 | A | A |
| | Phe-[Cys-Lys-Arg-Aib- | | | | | | |
| | Asn-Cys]-Tle-Thr-NH₂ | | | | | | |
| PSM-0449 | DOTA-Cmp-Thr-Aib- | I | 1732.875 | 1732.876 | 3.89 | A | A |
| | Phe-[Cys-Lys-Arg-Aib- | | | | | | |
| | Asn-Cys]-Tle-Bal-NH₂ | | | | | | |
| PSM-0450 | Iva-Aib-Phe-[Cys-Lys- | I | 1234.632 | 1234.614 | 5.36 | A | A |
| | Arg-Aib-Asn-Cys]-Tle- | | | | | | |
| | Thr-NH₂ | | | | | | |
| PSM-0451 | Ac-Thr-Aib-Phe-[Cys- | I | 1307.648 | 1307.647 | 4.20 | B | B |
| | Nmk-Arg-Aib-Asn- | | | | | | |
| | Cys]-Tle-Thr-NH₂ | | | | | | |
| PSM-0452 | nBuCAyl-Thr-Aib-Pcf- | IV | 2024.010 | 2024.014 | 5.66 | A | A |
| | [Cys-Lys-Arg-Aib-Asn- | | | | | | |
| | Cys]-Tle-Thr- | | | | | | |
| | lys(DOTA-Cmp)-NH₂ | | | | | | |
| PSM-0453 | Ac-Thr-Aib-Phe-[Cys- | I | 1305.669 | 1305.668 | 5.38 | A | A |
| | Lys-Arg-Aib-Asn-Cys]- | | | | | | |
| | Tle-Leu-NH₂ | | | | | | |
| PSM-0454 | DOTA-Cmp-Thr-Aib- | I | 1764.865 | 1764.858 | 3.28 | C | C |
| | Ppa-[Cys-Lys-Cit-Aib- | | | | | | |
| | Asn-Cys]-Tle-Thr-NH₂ | | | | | | |
| PSM-0455 | DOTA-Cmp-Nmt-Ala- | I | 1796.847 | 1796.840 | 4.19 | B | C |
| | Pcf-[Cys-Lys-Arg-Aib- | | | | | | |
| | Asn-Cys]-Tle-Thr-NH₂ | | | | | | |
| PSM-0456 | DOTA-Cmp-Thr-Aib- | XII | 1752.857 | 1752.852 | 3.94 | A | B |
| | Pcf-[Cys-Lys-Arg-Aib- | | | | | | |
| | Asn-Cys]-Tle-en(Me) | | | | | | |
| PSM-0458 | DOTA-Pamb-Aib-Phe- | I | 1568.759 | 1568.755 | 4.35 | A | B |
| | [Cys-Lys-Arg-Aib-Asn- | | | | | | |
| | Cys]-Tle-NH₂ | | | | | | |
| PSM-0459 | DOTA-Cmp-Thr-Aib- | I | 1714.865 | 1714.869 | 3.47 | B | B |
| | Mnf-[Cys-Lys(Me)- | | | | | | |
| | Arg(Me)-Aib-Asn-Cys]- | | | | | | |
| | Tle-NH₂ | | | | | | |
| PSM-0460 | Ac-Thr-Ala-Phe-[Cys- | I | 1406.644 | 1406.706 | 4.17 | A | B |
| | Lys-Arg-Pro-Asn-Cys]- | | | | | | |
| | Nle-Thr-Asp-NH₂ | | | | | | |
| PSM-0461 | Hib-Pcf-[Cys-Lys-Arg- | I | 1185.519 | 1185.518 | 4.22 | A | B |
| | Aib-Asn-Cys]-Tle-Thr- | | | | | | |
| | NH₂ | | | | | | |
| PSM-0462 | Hex-Thr-Aib-Phe-[Cys- | II | 2152.138 | 2152.285 | 5.71 | A | A |
| | Lys-Arg-Ala-Asn-Cys]- | | | | | | |
| | Tle-Thr-Ttds- | | | | | | |
| | lys(DOTA)-NH₂ | | | | | | |
| PSM-0464 | Ac-Thr-Aib-Phe-[Cys- | III | 1862.950 | 1863.058 | 3.85 | A | A |
| | Lys-Arg-Lys(DOTA- | | | | | | |
| | APAc)-Asn-Cys]-Tle- | | | | | | |
| | Thr-NH₂ | | | | | | |
| PSM-0465 | Ac-Thr-Aib-Phe-[Cys- | III | 2025.039 | 2025.194 | 4.35 | A | A |
| | Lys-Arg-Lys(DOTA- | | | | | | |
| | Ttds)-Asn-Cys]-Tle- | | | | | | |
| | Thr-NH₂ | | | | | | |
| PSM-0466 | DOTA-Cmp-Thr-Aib- | I | 1838.857 | 1838.997 | 4.61 | B | A |
| | Pcf-[Cys-Lys(Ac)-Arg- | | | | | | |
| | Aib-Asn-Cys]-Tle-Thr- | | | | | | |
| | NH₂ | | | | | | |
| PSM-0467 | DOTA-Cmp-Thr-Aib- | I | 1766.836 | 1766.839 | 4.36 | A | A |
| | Pcf-[Cys-Lys-Arg-Aib- | | | | | | |
| | Asn-Cys]-Tle-Bal-NH₂ | | | | | | |
| PSM-0468 | EuDOTA-Cmp-Thr- | XXd | 1944.743 | 1944.845 | 11.88 | A | n.d. |
| | Aib-Pcf-[Cys-Lys-Arg- | | | | | | |
| | Aib-Asn-Cys]-Tle-Thr- | | | | | | |
| | NH₂ | | | | | | |
| PSM-0469 | Ac-Ser-Ala-Phe-[Cys- | I | 1366.612 | 1366.651 | 4.14 | A | B |
| | Lys-Arg-Ala-Asn-Cys]- | | | | | | |
| | Nle-Thr-Asp-NH₂ | | | | | | |
| PSM-0470 | nBuCAyl-Thr-Aib-Mcf- | IV | 2024.010 | 2024.005 | 5.76 | A | A |
| | [Cys-Lys-Arg-Aib-Asn- | | | | | | |
| | Cys]-Tle-Thr-Cmp- | | | | | | |
| | lys(DOTA)-NH₂ | | | | | | |
| PSM-0471 | Ac-Thr-Aib-Phe-[Cys- | II | 1926.944 | 1927.041 | 4.52 | A | A |
| | Lys-Arg-Aib-Asn-Cys]- | | | | | | |
| | Tle-Thr-Pab- | | | | | | |
| | lys(DOTA)-NH₂ | | | | | | |
| PSM-0472 | Ac-Aib-Pcf-[Cys- | VIII | 1568.736 | 1568.735 | 4.15 | A | A |
| | Lys(Me)-Arg-Aib-Asn- | | | | | | |
| | Cys]-Tle-en-DOTA | | | | | | |
| PSM-0474 | Ac-Thr-Ala-Phe-[Cys- | I | 1380.628 | 1380.669 | 4.29 | C | D |
| | Lys-Arg-ala-Asn-Cys]- | | | | | | |
| | Nle-Thr-Asp-NH₂ | | | | | | |
| PSM-0475 | Ac-Thr-Ala-Phe-[Cys- | I | 1323.570 | 1323.630 | 4.65 | C | D |
| | Ala-Arg-Ala-Asn-Cys]- | | | | | | |
| | Nle-Thr-Asp-NH₂ | | | | | | |
| PSM-0476 | Ac-Thr-Ala-Phe-[Cys- | I | 1350.617 | 1350.677 | 4.30 | B | C |
| | Lys-Arg-Ala-Asn-Cys]- | | | | | | |
| | Nle-Ala-Asp-NH₂ | | | | | | |
| PSM-0477 | Ac-Thr-Aib-Phe-[Cys- | I | 1249.606 | 1249.606 | 4.47 | A | A |
| | Lys-Arg-Aib-Asn-Cys]- | | | | | | |
| | Tle-Gly-NH₂ | | | | | | |
| PSM-0478 | Ac-Thr-Aib-Phe-[Cys- | I | 1294.616 | 1294.594 | 4.19 | A | A |
| | Lys-Arg-Aib-Asp-Cys]- | | | | | | |
| | Tle-Thr-NH₂ | | | | | | |
| PSM-0479 | Ac-Thr-Aib-Phe-[Cys- | I | 1236.611 | 1236.591 | 4.11 | A | A |
| | Lys-Arg-Aib-Gly-Cys]- | | | | | | |
| | Tle-Thr-NH₂ | | | | | | |
| PSM-0480 | DOTA-Ttds-Thr-Ala- | I | 1810.907 | 1810.924 | 4.31 | C | C |
| | Phe-[Cys-Lys-Arg-Ala- | | | | | | |
| | Asn-Cys]-Leu-NH₂ | | | | | | |
| PSM-0481 | Ac-Aib-Pcf-[Cys- | VIII | 1582.752 | 1582.750 | 4.21 | A | A |
| | Lys(Me)-Arg(Me)-Aib- | | | | | | |
| | Asn-Cys]-Tle-en-DOTA | | | | | | |
| PSM-0482 | Ac-Thr-Aib-Pcf-[Cys- | II | 1966.952 | 1967.084 | 4.55 | A | A |
| | Lys-Arg-Aib-Asn-Cys]- | | | | | | |
| | Tle-Thr-Cmp- | | | | | | |
| | lys(DOTA)-NH₂ | | | | | | |
| PSM-0483 | DOTA-Cmp-Thr-Aib- | I | 1853.917 | 1853.910 | 3.63 | A | B |
| | Pcf-[Cys-KMe3- | | | | | | |
| | Arg(Me)-Aib-Asn-Cys]- | | | | | | |
| | Tle-Thr-NH₂ | | | | | | |
| PSM-0484 | nBuCAyl-Thr-Aib-Phe- | IV | 1990.049 | 1990.043 | 5.31 | A | A |
| | [Cys-Lys-Arg-Aib-Asn- | | | | | | |
| | Cys]-Tle-Thr-Cmp- | | | | | | |
| | lys(DOTA)-NH₂ | | | | | | |
| PSM-0485 | DOTA-Cmp-Thr-Aib- | I | 1810.826 | 1810.836 | 4.44 | A | A |
| | Pcf-[Cys-Hgn-Arg-Aib- | | | | | | |
| | Asn-Cys]-Tle-Thr-NH₂ | | | | | | |
| PSM-0486 | Ac-Thr-Aib-Mcf-[Cys- | II | 1966.952 | 1967.077 | 4.64 | A | A |
| | Lys-Arg-Aib-Asn-Cys]- | | | | | | |
| | Tle-Thr-Cmp- | | | | | | |
| | lys(DOTA)-NH₂ | | | | | | |
| PSM-0488 | Ac-Thr-Aib-Tyr-[Cys- | I | 1424.654 | 1424.681 | 3.61 | A | B |
| | Lys-Arg-Aib-Asn-Cys]- | | | | | | |
| | Tle-Thr-Asp-NH₂ | | | | | | |
| PSM-0489 | DOTA-Cmp-Aib-Pcf- | IX | 1709.779 | 1709.778 | 4.54 | A | A |
| | [Cys-Lys-Gln(Gu)-Aib- | | | | | | |
| | Asn-Cys]-Tle-Thr-NH₂ | | | | | | |
| PSM-0490 | DOTA-Cmp-Thr-Aib- | I | 2022.925 | 2022.918 | 5.20 | B | A |
| | Pcf-[Cys-Lys(Bio)-Arg- | | | | | | |
| | Aib-Asn-Cys]-Tle-Thr- | | | | | | |
| | NH₂ | | | | | | |
| PSM-0491 | Ac-Ala-Ala-Phe-[Cys- | I | 1350.617 | 1350.680 | 4.31 | B | C |
| | Lys-Arg-Ala-Asn-Cys]- | | | | | | |
| | Nle-Thr-Asp-NH₂ | | | | | | |
| PSM-0492 | DOTA-Cmp-Thr-Aib- | I | 1762.886 | 1762.988 | 4.00 | A | A |
| | Phe-[Cys-Lys-Arg-Aib- | | | | | | |
| | Asn-Cys]-Tle-Thr-NH₂ | | | | | | |
| PSM-0493 | Ac-Thr-Aib-Pcf-[Cys- | VII | 1328.577 | 1328.573 | 5.04 | A | A |
| | Lys-Arg-Aib-Asn-Cys]- | | | | | | |
| | Tle-Thr-OH | | | | | | |
| PSM-0494 | Ac-Aib-Pcf-[Cys-Lys- | XV | 1879.884 | 1879.878 | 4.73 | A | B |
| | Gln(Gu)-Aib-Asn-Cys]- | | | | | | |
| | Tle-Thr-Cmp- | | | | | | |
| | lys(DOTA)-NH₂ | | | | | | |
| PSM-0495 | Crown-Cmp-Thr-Aib- | I | 1912.931 | 1912.920 | 3.54 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0496 | Ac-Thr-Aib-Pcf-[Cys- | I | 1343.577 | 1343.638 | 5.03 | A | B |
| | Lys-Cit-Aib-Glu-Cys]- | | | | | | |
| | Tle-Thr-NH₂ | | | | | | |
| PSM-0497 | DOTA-Cmp-Thr-Aib- | I | 1810.863 | 1810.859 | 4.49 | B | C |
| | Pcf-[Cys-Lys-Arg-Aib- | | | | | | |
| | Asn-Cys]-Nml-Thr-NH₂ | | | | | | |
| PSM-0498 | H-Met-Thr-Ala-Phe- | I | 1613.711 | 1613.715 | 3.94 | A | A |
| | [Cys-Lys-Arg-Ala-Asn- | | | | | | |
| | Cys]-Leu-Thr-Asp-Gly- | | | | | | |
| | Ser-NH₂ | | | | | | |
| PSM-0499 | DOTA-Cmp-Thr-Aib- | I | 1838.894 | 1839.001 | 3.41 | A | A |
| | Pcf-[Cys-Lys(Me)-Egd- | | | | | | |
| | Aib-Asn-Cys]-Tle-Thr- | | | | | | |
| | NH₂ | | | | | | |
| PSM-0500 | nBuCAyl-Thr-Aib-Pcf- | IV | 2024.994 | 2024.978 | 5.82 | A | A |
| | [Cys-Lys-Cit-Aib-Asn- | | | | | | |
| | Cys]-Tle-Thr- | | | | | | |
| | lys(DOTA-Cmp)-NH₂ | | | | | | |
| PSM-0501 | Ac-Thr-Aib-Phe-[Cys- | I | 1394.644 | 1394.690 | 4.50 | A | A |
| | Lys-Arg-Ala-Asn-Cys]- | | | | | | |
| | Nle-Thr-Asp-NH₂ | | | | | | |
| PSM-0502 | Hex-Thr-Aib-Phe-[Cys- | II | 2267.165 | 2267.300 | 5.63 | A | A |
| | Lys-Arg-Ala-Asn-Cys]- | | | | | | |
| | Tle-Thr-Asp-Ttds- | | | | | | |
| | lys(DOTA)-NH₂ | | | | | | |
| PSM-0503 | DOTA-Cmp-Thr-Aib- | I | 1703.885 | 1703.889 | 3.93 | A | B |
| | Mmf-[Cys-Lys(Me)- | | | | | | |
| | Arg(Me)-Aib-Asn-Cys]- | | | | | | |
| | Tle-NH₂ | | | | | | |
| PSM-0504 | DOTA-Ttds-Thr-Ala- | I | 2026.982 | 2026.983 | 4.23 | B | B |
| | Phe-[Cys-Lys-Arg-Ala- | | | | | | |
| | Asn-Cys]-Nle-Thr-Asp- | | | | | | |
| | NH₂ | | | | | | |
| PSM-0505 | DOTA-O₂0c-Thr-Aib- | I | 1782.876 | 1782.853 | 4.00 | A | A |
| | Phe-[Cys-Lys-Arg-Aib- | | | | | | |
| | Asn-Cys]-Tle-Thr-NH₂ | | | | | | |
| PSM-0506 | Ac-Aib-Phe-[Cys-Lys- | I | 1293.596 | 1293.603 | 4.06 | A | A |
| | Arg-Ala-Asn-Cys]-Tle- | | | | | | |
| | Thr-Asp-NH₂ | | | | | | |
| PSM-0507 | DOTA-Cmp-Aib-Phe- | I | 1661.838 | 1661.839 | 3.93 | A | B |
| | [Cys-Lys-Arg-Aib-Asn- | | | | | | |
| | Cys]-Tle-Thr-NH₂ | | | | | | |
| PSM-0508 | Ac-Thr-Pam-Phe-[Cys- | I | 1319.648 | 1319.719 | 4.48 | A | A |
| | Lys-Arg-Aib-Asn-Cys]- | | | | | | |
| | Tle-Thr-NH₂ | | | | | | |
| PSM-0509 | HPA-Aib-Pcf-[Cys-Lys- | II | 1895.915 | 1895.913 | 4.50 | A | A |
| | Arg-Aib-Asn-Cys]-Tle- | | | | | | |
| | Thr-Cmp-lys(DOTA)- | | | | | | |
| | NH₂ | | | | | | |
| PSM-0510 | Ac-Thr-Aib-Phe-[Cys- | XI | 1248.647 | 1248.645 | 5.99 | A | A |
| | Lys-Arg-Aib-Asn-Cys]- | | | | | | |
| | Tle-NHBu | | | | | | |
| PSM-0511 | DOTA-Cmp-Thr-Aib- | I | 1782.831 | 1782.830 | 4.03 | A | A |
| | Pcf-[Cys-Orn-Arg-Aib- | | | | | | |
| | Asn-Cys]-Tle-Thr-NH₂ | | | | | | |
| PSM-0512 | DOTA-Cmp-Thr-Aib- | I | 1764.865 | 1764.864 | 3.33 | B | B |
| | Opa-[Cys-Lys-Cit-Aib- | | | | | | |
| | Asn-Cys]-Tle-Thr-NH₂ | | | | | | |
| PSM-0513 | DOTA-Ahx-Aib-Phe- | I | 1649.838 | 1649.909 | 3.95 | A | A |
| | [Cys-Lys-Arg-Aib-Asn- | | | | | | |
| | Cys]-Tle-Thr-NH₂ | | | | | | |
| PSM-0514 | Ac-Thr-Aib-Pcf-[Cys- | I | 1297.583 | 1297.580 | 4.99 | A | A |
| | Lys-Arg-Aib-Asn-Cys]- | | | | | | |
| | Tle-Bal-NH₂ | | | | | | |
| PSM-0515 | Macropa-Cmp-Thr-Aib- | XXII | 2081.983 | 2081.973 | 4.21 | A | Nd |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0516 | Ac-Aib-Pcf-[Cys-Lys- | VI | 1554.720 | 1554.706 | 4.25 | A | A |
| | Arg-Aib-Asn-Cys]-Tle- | | | | | | |
| | en-DOTA | | | | | | |
| PSM-0517 | Ac-Thr-Aib-₂Ni-[Cys- | I | 1458.675 | 1458.760 | 5.20 | A | A |
| | Lys-Arg-Aib-Asn-Cys]- | | | | | | |
| | Tle-Thr-Asp-NH₂ | | | | | | |
| PSM-0518 | HYDAc-Aib-Phe-[Cys- | I | 1290.596 | 1290.665 | 3.90 | A | A |
| | Lys-Arg-Aib-Asn-Cys]- | | | | | | |
| | Tle-Thr-NH₂ | | | | | | |
| PSM-0519 | InDOTA-Cmp-Thr-Aib- | XXa | 1833.711 | 1833.708 | 4.70 | A | A |
| | Pcf-[Cys-Lys(Me)- | | | | | | |
| | Arg(Me)-Aib-Asn-Cys]- | | | | | | |
| | Tle-NH₂ | | | | | | |
| PSM-0520 | Ac-Thr-Aib-Phe-[Cys- | XXa | 1977.809 | 1977.815 | 4.46 | A | A |
| | Lys-Arg-Lys(InDOTA- | | | | | | |
| | O₂0c)-Asn-Cys]-Tle- | | | | | | |
| | Thr-NH₂ | | | | | | |
| PSM-0521 | DOTA-Ttds-Thr-Aib- | I | 1911.943 | 1912.039 | 4.49 | B | B |
| | Phe-[Cys-Lys-Gln-Aib- | | | | | | |
| | Asn-Cys]-Tle-Thr-NH₂ | | | | | | |
| PSM-0522 | Ac-Thr-Aib-Phe-[Cys- | II | 1940.960 | 1941.069 | 4.36 | A | A |
| | Lys-Arg-Aib-Asn-Cys]- | | | | | | |
| | Tle-Thr-Pamb- | | | | | | |
| | lys(DOTA)-NH₂ | | | | | | |
| PSM-0524 | Ac-Pcf-[Cys-Lys-Arg- | I | 927.361 | 927.359 | 2.60 | D | D |
| | Aib-Asn-Cys]-NH₂ | | | | | | |
| PSM-0525 | Ac-thr-Ala-Phe-[Cys- | I | 1380.628 | 1380.680 | 4.20 | D | D |
| | Lys-Arg-Ala-Asn-Cys]- | | | | | | |
| | Nle-Thr-Asp-NH₂ | | | | | | |
| PSM-0526 | LuDOTA-Cmp-Thr- | XXb | 1968.764 | 1968.765 | 5.09 | A | A |
| | Aib-Pcf-[Cys-Lys-Arg- | | | | | | |
| | Aib-Asn-Cys]-Tle-Thr- | | | | | | |
| | NH₂ | | | | | | |
| PSM-0527 | InDOTA-Cmp-Thr-Aib- | XXa | 1934.759 | 1934.757 | 4.53 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0529 | Ac-Thr-Aib-Phe-[Cys- | II | 1952.981 | 1953.076 | 4.21 | A | A |
| | Lys-Arg-Aib-Asn-Cys]- | | | | | | |
| | Tle-Thr-O₂Oc- | | | | | | |
| | lys(DOTA)-NH₂ | | | | | | |
| PSM-0530 | HPA-Aib-Phe-[Cys- | I | 1222.595 | 1222.574 | 3.95 | A | A |
| | Lys-Arg-Aib-Asn-Cys]- | | | | | | |
| | Tle-Thr-NH₂ | | | | | | |
| PSM-0531 | DOTA-Cmp-Thr-Aib- | I | 1782.820 | 1782.817 | 4.30 | A | A |
| | Pcf-[Cys-Lys-Hgn-Aib- | | | | | | |
| | Asn-Cys]-Tle-Thr-NH₂ | | | | | | |
| PSM-0532 | Ac-Thr-Aib-Phe-[Cys- | I | 1466.647 | 1466.674 | 4.63 | A | A |
| | Lys-Arg-Dtc-Asn-Cys]- | | | | | | |
| | Tle-Thr-Asp-NH₂ | | | | | | |
| PSM-0533 | Ac-Thr-Ala-Phe-[Cys- | II | 2197.087 | 2197.111 | 4.46 | B | B |
| | Lys-Arg-Ala-Asn-Cys]- | | | | | | |
| | Nle-Thr-Asp-Ttds- | | | | | | |
| | lys(DOTA)-NH₂ | | | | | | |
| PSM-0534 | DOTA-Thr-Aib-Pcf- | I | 1570.715 | 1570.716 | 4.45 | A | A |
| | [Cys-Lys-Arg-Aib-Asn- | | | | | | |
| | Cys]-Tle-NH₂ | | | | | | |
| PSM-0535 | Ac-Thr-Aib-Phe-[Cys- | I | 1394.644 | 1394.692 | 4.20 | B | A |
| | lys-Arg-Ala-Asn-Cys]- | | | | | | |
| | Tle-Thr-Asp-NH₂ | | | | | | |
| PSM-0536 | Ac-Thr-Aib-Pcf-[Cys- | XIII | 2906.198 | 2906.254 | 8.39 | Nd | Nd |
| | Lys(Me)-Arg(Me)-Aib- | | | | | | |
| | Asn-Cys]-Tle-Thr-Ttds- | | | | | | |
| | Ttds-AF488N3K-NH₂ | | | | | | |
| PSM-0537 | Ac-Thr-ala-Phe-[Cys- | I | 1380.628 | 1380.690 | 4.30 | C | D |
| | Lys-Arg-Ala-Asn-Cys]- | | | | | | |
| | Nle-Thr-Asp-NH₂ | | | | | | |
| PSM-0538 | DOTA-Cmp-Tle-Aib- | I | 1836.915 | 1837.004 | 4.53 | B | C |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0539 | DOTA-Thr-Aib-Phe- | I | 1607.791 | 1607.788 | 3.73 | A | B |
| | [Cys-Lys-Arg-Aib-Asn- | | | | | | |
| | Cys]-Tle-Bal-NH₂ | | | | | | |
| PSM-0540 | Ac-Thr-Aib-Phe-[Cys- | I | 1320.643 | 1320.642 | 4.17 | A | A |
| | Lys-Arg-Aib-Asn-Cys]- | | | | | | |
| | Tle-Gln-NH₂ | | | | | | |
| PSM-0541 | Ac-Thr-Aib-Phe-[Cys- | I | 1326.658 | 1326.762 | 5.31 | A | A |
| | Lys-Arg-Aib-Phe-Cys]- | | | | | | |
| | Tle-Thr-NH₂ | | | | | | |
| PSM-0542 | Ac-Thr-Aib-Phe-[Cys- | III | 2140.066 | 2140.188 | 4.18 | A | A |
| | Lys-Arg-Lys(DOTA- | | | | | | |
| | Ttds)-Asn-Cys]-Tle- | | | | | | |
| | Thr-Asp-NH₂ | | | | | | |
| PSM-0543 | DOTA-APAc-Thr-Aib- | I | 1777.897 | 1778.000 | 3.85 | A | A |
| | Phe-[Cys-Lys-Arg-Aib- | | | | | | |
| | Asn-Cys]-Tle-Thr-NH₂ | | | | | | |
| PSM-0544 | Ac-Thr-Ala-Phe-[Cys- | I | 1349.586 | 1349.625 | 4.80 | D | D |
| | Pro-Arg-Ala-Asn-Cys]- | | | | | | |
| | Nle-Thr-Asp-NH₂ | | | | | | |
| PSM-0545 | Ac-Thr-Aib-Phe-[Cys- | I | 1292.673 | 1292.781 | 5.10 | A | A |
| | Lys-Arg-Aib-Leu-Cys]- | | | | | | |
| | Tle-Thr-NH₂ | | | | | | |
| PSM-0546 | DOTA-Cmp-Thr-Aib- | XVII | 1811.821 | 1811.824 | 4.09 | A | A |
| | Pcf-[Cys-Lys-Urr-Aib- | I | | | | | |
| | Asn-Cys]-Tle-Thr-NH₂ | | | | | | |
| PSM-0547 | Ac-Thr-Pro-Phe-[Cys- | I | 1406.644 | 1406.687 | 4.33 | A | B |
| | Lys-Arg-Ala-Asn-Cys]- | | | | | | |
| | Nle-Thr-Asp-NH₂ | | | | | | |
| PSM-0548 | DOTA-Cmp-Thr-Aib- | I | 1810.863 | 1810.861 | 4.09 | B | B |
| | Pcf-[Cys-Nmk-Arg-Aib- | | | | | | |
| | Asn-Cys]-Tle-Thr-NH₂ | | | | | | |
| PSM-0549 | DOTA-Cmp-Thr-Aib- | I | 1757.857 | 1757.863 | 4.30 | A | B |
| | Ptf-[Cys-Lys(Me)- | | | | | | |
| | Arg(Me)-Aib-Asn-Cys]- | | | | | | |
| | Tle-NH₂ | | | | | | |
| PSM-0550 | Ac-Thr-Aib-Ptf-[Cys- | I | 1361.620 | 1361.618 | 4.64 | A | A |
| | Lys-Arg-Aib-Asn-Cys]- | | | | | | |
| | Tle-Thr-NH₂ | | | | | | |
| PSM-0551 | Ac-Thr-Aib-Mff-[Cys- | I | 1311.623 | 1311.628 | 3.88 | A | A |
| | Lys-Arg-Aib-Asn-Cys]- | | | | | | |
| | Tle-Thr-NH₂ | | | | | | |
| PSM-0552 | Ac-Thr-Aib-Phe-[Cys- | I | 1279.617 | 1279.623 | 4.17 | A | A |
| | Lys-Arg-Ala-Asn-Cys]- | | | | | | |
| | Tle-Thr-NH₂ | | | | | | |
| PSM-0553 | DOTA-Cmp-Thr-Aib- | I | 1707.860 | 1707.865 | 3.66 | A | B |
| | Mff-[Cys-Lys(Me)- | | | | | | |
| | Arg(Me)-Aib-Asn-Cys]- | | | | | | |
| | Tle-NH₂ | | | | | | |
| PSM-0554 | Ac-Thr-Aib-Pcf-[Cys- | VII | 1314.598 | 1314.594 | 4.98 | A | A |
| | Lys-Arg-Aib-Asn-Cys]- | | | | | | |
| | Tle-Throl-OH | | | | | | |
| PSM-0555 | Ac-Thr-Aib-Mnf-[Cys- | I | 1318.627 | 1318.632 | 3.76 | A | A |
| | Lys-Arg-Aib-Asn-Cys]- | | | | | | |
| | Tle-Thr-NH₂ | | | | | | |
| PSM-0556 | Ac-Thr-Aib-Phe-[Cys- | I | 1339.653 | 1339.760 | 5.35 | A | A |
| | Lys-Arg-Aib-Asn-Cys]- | | | | | | |
| | Tle-Phe-NH₂ | | | | | | |
| PSM-0558 | Ac-Thr-Aib-Ocf-[Cys- | I | 1442.620 | 1442.648 | 4.51 | A | B |
| | Lys-Arg-Aib-Asn-Cys]- | | | | | | |
| | Tle-Thr-Asp-NH₂ | | | | | | |
| PSM-0559 | Ac-Aib-Pcf-[Cys-Lys- | I | 1226.546 | 1226.525 | 4.75 | A | A |
| | Arg-Aib-Asn-Cys]-Tle- | | | | | | |
| | Thr-NH₂ | | | | | | |
| PSM-0560 | Ac-Thr-Aib-Phe-[Cys- | I | 1206.600 | 1206.599 | 4.97 | A | B |
| | Lys-Arg-Aib-Asn-Cys]- | | | | | | |
| | Npg-NH₂ | | | | | | |
| PSM-0561 | Ac-Thr-Aib-Phe-[Cys- | XXa | 1947.762 | 1947.846 | 4.18 | A | A |
| | Lys-Arg-Lys(InDOTA)- | | | | | | |
| | Asn-Cys]-Tle-Thr-Asp- | | | | | | |
| | NH₂ | | | | | | |
| PSM-0562 | Ac-Thr-Ala-Phe-[Cys- | I | 1336.638 | 1336.697 | 4.37 | B | C |
| | Lys-Arg-Ala-Asn-Cys]- | | | | | | |
| | Nle-Thr-Ala-NH₂ | | | | | | |
| PSM-0563 | Ac-Thr-Aib-Phe-[Cys- | I | 1308.632 | 1308.609 | 4.24 | A | B |
| | Lys-Arg-Aib-Glu-Cys]- | | | | | | |
| | Tle-Thr-NH₂ | | | | | | |
| PSM-0564 | Ac-Thr-Ala-Phe-[Cys- | I | 1321.580 | 1321.619 | 4.65 | D | D |
| | Lys-Pro-Ala-Asn-Cys]- | | | | | | |
| | Nle-Thr-Asp-NH₂ | | | | | | |
| PSM-0565 | Ac-Thr-Aib-Phe-[Cys- | I | 1394.644 | 1394.665 | 4.09 | A | A |
| | Lys-Arg-Ala-Asn-Cys]- | | | | | | |
| | Tle-Thr-Asp-NH₂ | | | | | | |
| PSM-0566 | DOTA-Cmp-Thr-Aib- | I | 1820.891 | 1820.891 | 3.82 | D | D |
| | Fac-[Cys-Lys-Arg-Aib- | | | | | | |
| | Asn-Cys]-Tle-Thr-NH₂ | | | | | | |
| PSM-0567 | Ac-Thr-Aib-Phe-[Smc- | XIX | 1408.659 | 1408.646 | 3.99 | A | A |
| | Lys-Arg-Ala-Asn-Cys]- | | | | | | |
| | Tle-Thr-Asp-NH₂ | | | | | | |
| | (alternative: Ac-Thr- | | | | | | |
| | Aib-Phe-[Cys-Lys-Arg- | | | | | | |
| | Ala-Asn-Smc]-Tle-Thr- | | | | | | |
| | Asp-NH₂) | | | | | | |
| PSM-0568 | Hex-Thr-Ala-Phe-[Cys- | II | 2253.15 | 2253.159 | 5.45 | A | B |
| | Lys-Arg-Ala-Asn-Cys]- | | | | | | |
| | Tle-Thr-Asp-Ttds- | | | | | | |
| | lys(DOTA)-NH₂ | | | | | | |
| PSM-0569 | Ac-Thr-Aib-Pcf-[Cys- | X | 1369.604 | 1369.575 | 4.82 | A | A |
| | Lys-Arg(Ac)-Aib-Asn- | | | | | | |
| | Cys]-Tle-Thr-NH₂ | | | | | | |
| PSM-0570 | Ac-Thr-Aib-Pcf-[Cys- | X | 1354.593 | 1354.593 | 6.54 | A | B |
| | Nle-Arg(Ac)-Aib-Asn- | | | | | | |
| | Cys]-Tle-Thr-NH₂ | | | | | | |
| PSM-0571 | nBuCAyl-Thr-Aib-Mcf- | IV | 2059.015 | 2059.011 | 5.94 | B | B |
| | [Cys-Lys-Opy-Aib-Asn- | | | | | | |
| | Cys]-Tle-Thr-Cmp- | | | | | | |
| | lys(DOTA)-NH₂ | | | | | | |
| PSM-0572 | nBuCAyl-Thr-Aib-Phe- | XXb | 2161.967 | 2161.975 | 5.74 | A | A |
| | [Cys-Lys-Arg-Aib-Asn- | | | | | | |
| | Cys]-Tle-Thr-Cmp- | | | | | | |
| | lys(LuDOTA)-NH₂ | | | | | | |
| PSM-0573 | DOTA-Thr-Aib-Pcf- | I | 1671.763 | 1671.757 | 4.39 | A | A |
| | [Cys-Lys-Arg-Aib-Asn- | | | | | | |
| | Cys]-Tle-Thr-NH₂ | | | | | | |
| PSM-0574 | DOTA-Pamb-Aib-Phe- | I | 1639.796 | 1639.797 | 4.1 | A | B |
| | [Cys-Lys-Arg-Aib-Asn- | | | | | | |
| | Cys]-Tle-Bal-NH₂ | | | | | | |
| PSM-0575 | Bio-Ttds-Ttds-Thr-Aib- | I | 2116.029 | 2116.022 | 5.43 | A | A |
| | Pcf-[Cys-Lys-Arg-Aib- | | | | | | |
| | Asn-Cys]-Tle-Thr-NH₂ | | | | | | |
| PSM-0576 | Hex-Thr-Aib-Phe-[Cys- | II | 2166.154 | 2166.261 | 5.93 | A | A |
| | Lys-Arg-Aib-Asn-Cys]- | | | | | | |
| | Tle-Thr-Ttds- | | | | | | |
| | lys(DOTA)-NH₂ | | | | | | |
| PSM-0577 | HO-Succinyl-Aib-Phe- | I | 1250.59 | 1250.568 | 4.12 | A | B |
| | [Cys-Lys-Arg-Aib-Asn- | | | | | | |
| | Cys]-Tle-Thr-NH₂ | | | | | | |
| PSM-0578 | Ac-Thr-Ala-Phe-[Cys- | I | 1394.644 | 1394.709 | 4.46 | A | B |
| | Lys-Arg-Aib-Asn-Cys]- | | | | | | |
| | Nle-Thr-Asp-NH₂ | | | | | | |
| PSM-0579 | DOTA-Cmp-Thr-Aib- | XVII | 1867.884 | 1867.881 | 4.4 | A | A |
| | Pcf-[Cys-Lys- | | | | | | |
| | Arg(EtCAyl)-Aib-Asn- | | | | | | |
| | Cys]-Tle-Thr-NH₂ | | | | | | |
| PSM-0580 | DOTA-Thr-Aib-Phe- | I | 1637.802 | 1637.897 | 3.8 | A | A |
| | [Cys-Lys-Arg-Aib-Asn- | | | | | | |
| | Cys]-Tle-Thr-NH₂ | | | | | | |
| PSM-0581 | Ac-Thr-Ala-Phe-[Cys- | I | 1295.564 | 1295.628 | 4.48 | C | D |
| | Lys-Ala-Ala-Asn-Cys]- | | | | | | |
| | Nle-Thr-Asp-NH₂ | | | | | | |
| PSM-0582 | Ac-Thr-Aib-Phe-[Cys- | III | 1835.939 | 1836.016 | 4.16 | A | A |
| | Lys-Arg-Lys(DOTA- | | | | | | |
| | Ahx)-Asn-Cys]-Tle- | | | | | | |
| | Thr-NH₂ | | | | | | |
| PSM-0583 | DOTA-Cmp-Thr-Aib- | I | 1796.847 | 1796.909 | 4.29 | A | A |
| | Mcf-[Cys-Lys-Arg-Aib- | | | | | | |
| | Asn-Cys]-Tle-Thr-NH₂ | | | | | | |
| PSM-0584 | Ac-Thr-Aib-Pcf-[Cys- | I | 1327.618 | 1327.656 | 5.84 | A | A |
| | Lys-Cit-Aib-Leu-Cys]- | | | | | | |
| | Tle-Thr-NH₂ | | | | | | |
| PSM-0585 | DOTA-Cmp-Thr-Aib- | I | 1796.847 | 1796.841 | 4.11 | A | B |
| | Pcf-[Cys-Lys-Arg-Ala- | | | | | | |
| | Nmn-Cys]-Tle-Thr-NH₂ | | | | | | |
| PSM-0586 | Ac-Thr-Ala-Ala-[Cys- | I | 1304.597 | 1304.594 | 3.08 | D | D |
| | Lys-Arg-Ala-Asn-Cys]- | | | | | | |
| | Nle-Thr-Asp-NH₂ | | | | | | |
| PSM-0587 | Ac-Thr-Aib-Trp-[Cys- | I | 1447.670 | 1447.750 | 4.31 | A | A |
| | Lys-Arg-Aib-Asn-Cys]- | | | | | | |
| | Tle-Thr-Asp-NH₂ | | | | | | |
| PSM-0589 | Ac-Thr-Aib-Phe-[Cys- | I | 1365.669 | 1365.780 | 5.33 | A | A |
| | Lys-Arg-Aib-Trp-Cys]- | | | | | | |
| | Tle-Thr-NH₂ | | | | | | |
| PSM-0590 | DOTA-Cmp-Thr-Aib- | XXI | 1838.894 | 1839.002 | 3.47 | A | A |
| | Pcf-[Cys-Lys(Me)- | | | | | | |
| | RMe₂a-Aib-Asn-Cys]- | | | | | | |
| | Tle-Thr-NH₂ | | | | | | |
| PSM-0591 | Ac-[Cys-Lys-Arg-Ala- | II | 1877.934 | 1877.935 | 3.52 | D | D |
| | Asn-Cys]-Leu-Thr-Asp- | | | | | | |
| | Ttds-lys(DOTA)-NH₂ | | | | | | |
| PSM-0592 | DOTA-Cmp-Thr-Aib- | I | 1824.878 | 1824.929 | 3.44 | A | Nd |
| | Pcf-[Cys-Lys(Me)- | | | | | | |
| | RMel-Aib-Asn-Cys]- | | | | | | |
| | Tle-Thr-NH₂ | | | | | | |
| PSM-0593 | DOTAM-Cmp-Thr-Aib- | I | 1821.926 | 1821.922 | 4.233 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0594 | DOTA-Cmp-Thr-Aib- | I | 1611.730 | 1611.730 | 4.11 | C | D |
| | Pcf-[Cys-Lys(Me)- | | | | | | |
| | Arg(Me)-Aib-Asn-Cys]- | | | | | | |
| | OH | | | | | | |
| PSM-0595 | BIDOTA-Cmp-Thr-Aib- | XXe | 2030.835 | 2030.835 | 5.06 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0596 | LaDOTA-Cmp-Thr- | XXf | 1959.762 | 1959.762 | 4.98 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0597 | PbDOTA-Cmp-Thr- | XXg | 2026.836 | 2026.834 | 4.985 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0598 | BIDOTAM-Cmp-Thr- | XXe | 2030.907 | 2030.890 | 3.95 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0599 | InDOTAM-Cmp-Thr- | XXa | 1934.830 | 1934.812 | 3.99 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0600 | PbDOTAM-Cmp-Thr- | XXg | 2025.899 | 2025.889 | 4.29 | A | A |
| | Aib-Pcf-[Cys-Lys(Me)- | | | | | | |
| | Arg(Me)-Aib-Asn-Cys]- | | | | | | |
| | Tle-Thr-NH₂ | | | | | | |
| PSM-0601 | LSC-Cmp-Thr-Aib-Pcf- | I | 1823.894 | 1823.905 | 4.48 | A | A |
| | [Cys-Lys(Me)-Arg(Me)- | | | | | | |
| | Aib-Asn-Cys]-Tle-Thr- | | | | | | |
| | NH₂ | | | | | | |
| PSM-0602 | PbLSC-Cmp-Thr-Aib- | XXg | 2025.852 | 2025.848 | 4.70 | Nd | Nd |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0603 | BiLSC-Cmp-Thr-Aib- | XXe | 2029.851 | 2029.850 | 4.49 | Nd | Nd |
| | Pcf-[Cys-Lys(Me)- | | | | | | |
| | Arg(Me)-Aib-Asn-Cys]- | | | | | | |
| | Tle-Thr-NH₂ | | | | | | |
| PSM-0604 | InLSC-Cmp-Thr-Aib- | XXa | 1933.775 | 1933.769 | 4.59 | A | A |
| | Pcf-[Cys-Lys(Me)- | | | | | | |
| | Arg(Me)-Aib-Asn-Cys]- | | | | | | |
| | Tle-Thr-NH₂ | | | | | | |
| PSM-605 | DOTA-Cmp-Thr-Aib- | V | 1732.912 | 1732.924 | 3.78 | A | A |
| | Phe-[Cys-Lys(Me)- | | | | | | |
| | Arg(Me)-Aib-Asn-Cys]- | | | | | | |
| | Tle-en | | | | | | |
| PSM-606 | DOTA-Cmp-Thr-Aib- | I | 1794.868 | 1794.876 | 4.48 | A | A |
| | Pcf-[Cys-Lys(Me)- | | | | | | |
| | Arg(Me)-Aib-Asn-Cys]- | | | | | | |
| | Tle-Bal-NH₂ | | | | | | |

### Example 33: Enzyme Inhibition Assay

PSMA possesses glutamate-preferring carboxypeptidase activity (Carter R E, et al., Proc Natl Acad Sci USA. 1996 Jan. 23; 93(2): 749-753). This peptidase activity hydrolyzes γ-peptide bonds between N-acetyl amino acid and glutamate of its substrate.

The carboxypeptidase inhibition of recombinant human PSMA by test compounds was assessed using a commercial fluorescence-based PSMA activity inhibition assay. The known PSMA inhibitor 2-PMPA (2-phosphonomethyl pentanedioic acid) was used as control compound.

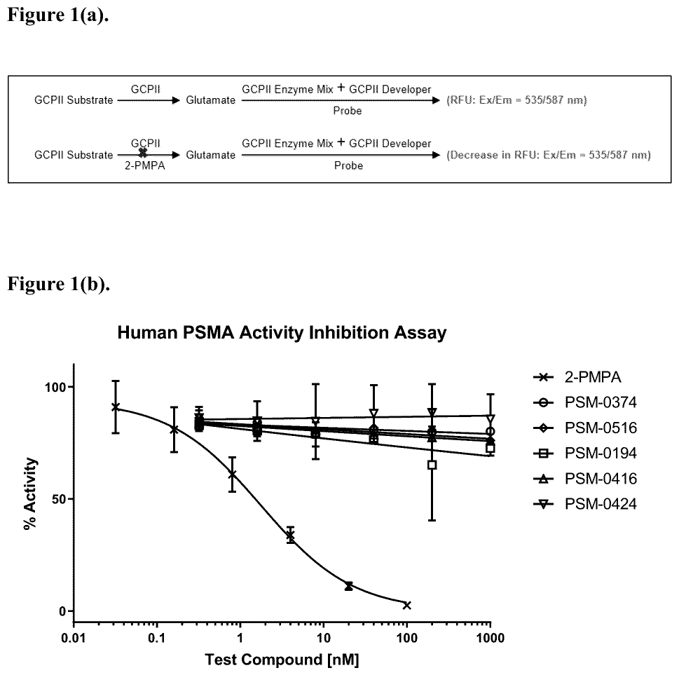

The employed Glutamate Carboxypeptidase II (GCPII) Inhibitor Screening Kit (BioVision, Cat #K440-100) is a fluorescence-based, enzymatic in vitro assay to measure the potency of PSMA inhibitors. The provided substrate is transaminated in the presence of PSMA producing glutamate. The detection system is based on an enzymatic reaction in which a fluorogenic probe is reduced, generating a stable signal. Using a potent PSMA inhibitor, enzymatic activity is arrested, thus generating a lower fluorometric signal (FIG. 1(a)).

For the assay, recombinant human PSMA was diluted in GCPII Assay Buffer as recommended in the assay protocol. Serial dilutions of the test compounds were prepared, starting from 10 μM (final reaction concentration of 1 μM). In a 96-well assay plate, 40 μL of the PSMA working solution was mixed with 30 μL of GCPII Assay Buffer. Next, 10 μL of the serially diluted test compounds were added. The mixture was incubated for 20 min at 37° C. Subsequently, 20 μL of a reaction mix solution was added. The fluorescence signal increased due to fluorophore reduction, which is directly proportional to the produced amount of glutamate, was measured in kinetic mode for 90 minutes at 37° C. in a SpectraMax M5 plate reader. RFU/see was assessed by the SoftMax Pro software and plotted against peptide concentrations. Four-parameter logistic (4PL) curve fitting and pIC₅₀ calculations were performed using the GraphPad Prism software. FIG. 1(a) shows an illustration of the PSMA activity inhibition assay.

The compounds tested did not inhibit the measured enzymatic activity of PSMA at a concentration of up to 1 μM (FIG. 1(b), Table 7). Therefore, the test compounds do not block the active site or inhibit the enzymatic activity of PSMA in a different (e.g. allosteric) manner.

TABLE 7
pIC₅₀ for PSMA inhibition

| Com- | | |
|---|---|---|
| pound | | |
| ID | Sequence/Name | PIC₅₀ |
| PSM- | DOTA-Cmp-Thr-Aib-Pcf- | <6 no |
| 0194 | [Cys-Lys(Me)-Arg(Me)- | inhibition |
| | Aib-Asn-Cys]-Tle-Thr- | at 1 μM |
| | NH₂ | |
| PSM- | Ac-Thr-Aib-Phe-[Cys- | <6 no |
| 0374 | Lys-Arg-Aib-Asn-Cys]- | inhibition |
| | Tle-Thr-Asp-NH₂ | at 1 μM |
| PSM- | DOTA-Cmp-Thr-Aib-Pcf- | <6 no |
| 0416 | [Cys-Lys-Arg-Glu-Asn- | inhibition |
| | Cys]-Tle-Thr-NH₂ | at 1 μM |
| PSM- | Ac-Aib-Pcf-[Cys-Lys- | <6 no |
| 0424 | Arg-Aib-Asn-Cys]-Tle- | inhibition |
| | NH₂ | at 1 μM |
| PSM- | Ac-Aib-Pcf-[Cys-Lys- | <6 no |
| 0516 | Arg-Aib-Asn-Cys]-Tle- | inhibition |
| | en-DOTA | at 1 μM |
| 2-PMPA | 2-phosphonomethyl | 8.5 to 9.2 |
| | pentanedioic acid | |

### Example 34: ¹¹¹In-Labeling

In order to serve as a diagnostically, therapeutically, or theranostically active agent, a compound needs to be labeled with a radioactive isotope. The labeling procedure needs to be appropriate to ensure a high radiochemical yield and purity of the radiolabeled compound of the invention. This example shows that the compounds of the present invention are appropriate for radiolabeling and can be labeled in high radiochemical yield and purity.

˜90 MBq of ¹¹¹InCl₃ (in 0.02 M HCl; Curium, Germany) were mixed with 1 nmol of compound (200 μM stock solution diluted from 10 mM stock solution in DMSO with 0.1 M HEPES) per 30 MBq and buffer (1 M sodium acetate pH 5 or 1 M ammonium acetate pH 5) at a final buffer concentration of 0.1 M. Three different combinations of buffer and reaction time and temperatures were tried for labeling.
1. 1 M sodium acetate buffer at pH 5, 80° C., 25 min
2. 1 M ammonium acetate buffer at pH 5, 80° C., 25 min
3. 1 M ammonium acetate buffer at pH 5, 90° C., 15 min

After cooling down, ascorbic acid (Woerwag Pharma, Germany), DTPA (Heyl, Germany) and TWEEN-20 were added at a final concentration of 25 mg/mL, 0.1 mg/mL and 0.1%, respectively.

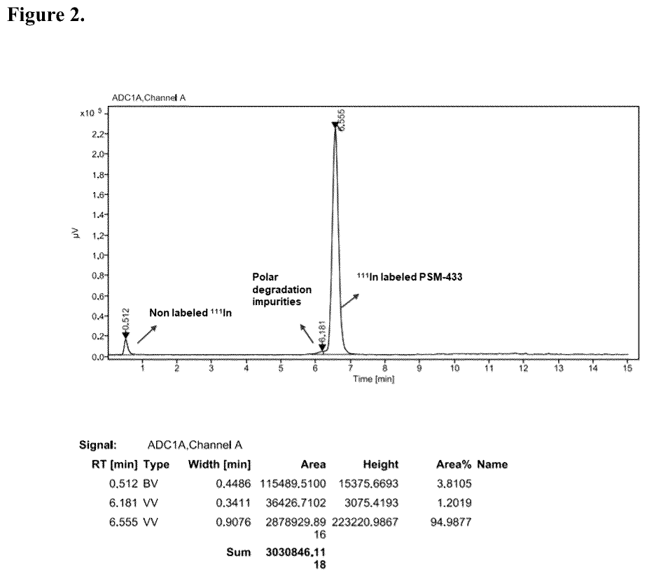

Radiochemical purity was analyzed by HPLC. 5 μl of diluted labeling solution was analyzed with a Poroshell SB—C18 2.7 μm, 2.1×50 mm (Agilent). Fluent A: H₂O, 0.1% TFA eluent B: MeCN, gradient from 5% B to 70% B within 15 min, flow rate 0.5 mL/min; detector: NaI (Raytest), DAD 230 nm. The peak eluting with the dead volume represents free radionuclide, the peak eluting with the peptide-specific retention time as determined with an unlabeled sample represents radiolabeled compound. Radiochemical purity was usually >90% at end of synthesis. Table 9 lists the compounds labeled with ¹¹¹In and the conditions used for labeling each compound. An exemplary radiochromatogram is shown in FIG. 2 with all peaks labeled with their retention times.

TABLE 9
Labeling conditions by compound

Labeling conditions

| | | temperature | time |
|---|---|---|---|
| Compound ID | buffer | [° C.] | [min] |
| PSM-0190 | 1M NH₄OAc | 80 | 25 |
| PSM-0194 | 1M NH₄OAc | 90 | 15 |
| PSM-0199 | 1M NH₄OAc | 90 | 15 |
| PSM-0218 | 1M NaOAc | 80 | 25 |
| PSM-0234 | 1M NaOAc | 80 | 25 |
| PSM-0237 | 1M NaOAc | 80 | 25 |
| PSM-0239 | 1M NH₄OAc | 80 | 25 |
| PSM-0241 | 1M NH₄OAc | 90 | 15 |
| PSM-0243 | 1M NaOAc | 80 | 25 |
| PSM-0267 | 1M NH₄OAc | 90 | 15 |
| PSM-0269 | 1M NH₄OAc | 90 | 15 |
| PSM-0273 | 1M NH₄OAc | 90 | 15 |
| PSM-0283 | 1M NH₄OAc | 90 | 15 |
| PSM-0285 | 1M NaOAc | 80 | 25 |
| PSM-0301 | 1M NaOAc | 80 | 25 |
| PSM-0339 | 1M NaOAc | 80 | 25 |
| PSM-0361 | 1M NH₄OAc | 90 | 15 |
| PSM-0365 | 1M NaOAc | 80 | 25 |
| PSM-0371 | 1M NH₄OAc | 80 | 25 |
| PSM-0377 | 1M NH₄OAc | 90 | 15 |
| PSM-0384 | 1M NH₄OAc | 90 | 15 |
| PSM-0416 | 1M NH₄OAc | 90 | 15 |
| PSM-0428 | 1M NaOAc | 80 | 25 |
| PSM-0433 | 1M NH₄OAc | 90 | 15 |
| PSM-0449 | 1M NH₄OAc | 90 | 15 |
| PSM-0467 | 1M NH₄OAc | 90 | 15 |
| PSM-0481 | 1M NH₄OAc | 90 | 15 |
| PSM-0492 | 1M NH₄OAc | 80 | 25 |
| PSM-0516 | 1M NH₄OAc | 90 | 15 |
| PSM-0531 | 1M NH₄OAc | 90 | 15 |
| PSM-0534 | 1M NH₄OAc | 90 | 15 |
| PSM-0573 | 1M NH₄OAc | 90 | 15 |
| PSM-0579 | 1M NH₄OAc | 90 | 15 |
| PSM-0580 | 1M NH₄OAc | 80 | 25 |

### Example 35: Imaging

Radioactively labeled compounds can be detected by imaging methods such as SPECT and PET. Furthermore, the data acquired by such techniques can be confirmed by direct measurement of radioactivity contained in the individual organs prepared from an animal injected with a radioactively labeled compound of the disclosure. Thus, the biodistribution (the measurement of radioactivity in individual organs) of a radioactively labeled compound can be determined and analyzed. This example shows that the compounds of the present disclosure show a biodistribution appropriate for diagnostic imaging and therapeutic treatment of tumors.

All animal experiments were conducted in compliance with the German animal protection laws. Male swiss or NMRI nude mice (6-8 weeks old, Janvier Labs, France) were inoculated with 5×10⁶ PC3-PIP cells in the right shoulder. For selected compounds an additional model was used, here male NMRI nude mice (6-8 weeks old, Janvier Labs, France) were inoculated with 5×10⁶ or/1×10⁷ C4-2 cells in the right shoulder. When tumors reached an appropriate size, the mice received ˜30 MBq ¹¹¹In-labeled compounds of the disclosure (diluted to 100 μL with PBS) administered intravenously via the tail vein. Images were obtained on a NanoSPECT/computed tomography system (Mediso Medical Imaging Systems, Budapest, Hungary) using exemplarily the following acquisition and reconstruction parameters (Table 10).

TABLE 10
Acquisition and reconstruction parameters
of NanoSPECT/computed tomography imaging
Acquistion parameters SPECT

| | |
|---|---|
| System | NanoSPECT/computed |
| | tomography ™ |
| Scan range | whole body, 3-bed |
| | holder (mouse hotel) |
| Time per projection | 60 s or 90 s |
| Aperture model, | Aperture #2, 1.5 mm |
| pinhole diameter | |

Reconstruction parameters

| | |
|---|---|
| Method | HiSPECT (Scivis), iterative |
| | reconstruction |
| Smoothing | 35% |
| Iterations | 9 |
| Voxel size | 0.15 mm × 0.15 mm × 0.15 mm |

Acquisition parameters CT

| | |
|---|---|
| System | NanoSPECT/computed |
| | tomography ™ |
| Scan range | whole body, 3-bed |
| | holder (mouse hotel) |
| Scan duration | 7 minutes |
| Tube voltage | 45 kVp |
| Exposure time | 500 ms |
| Number of projections | 240 |

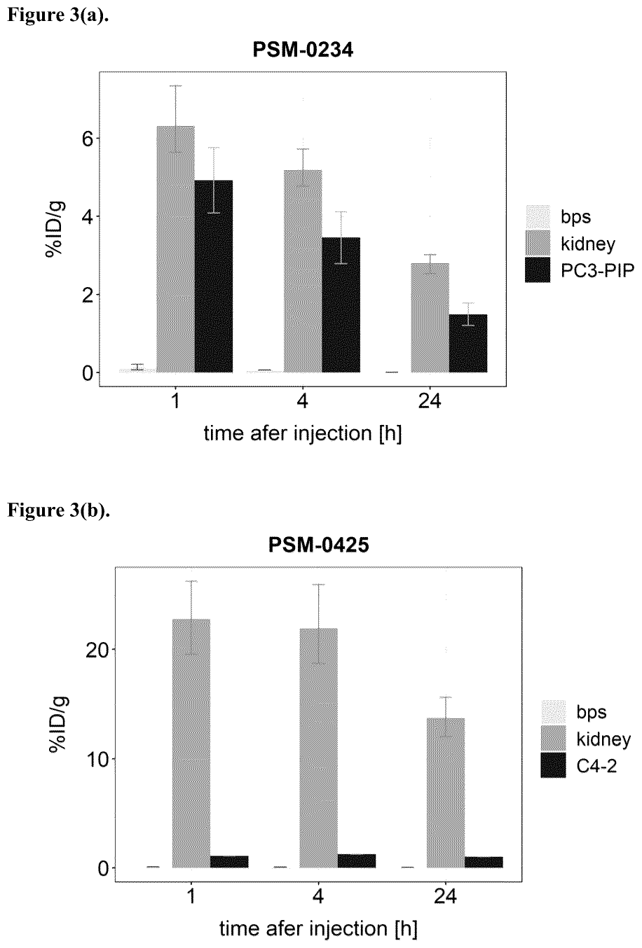

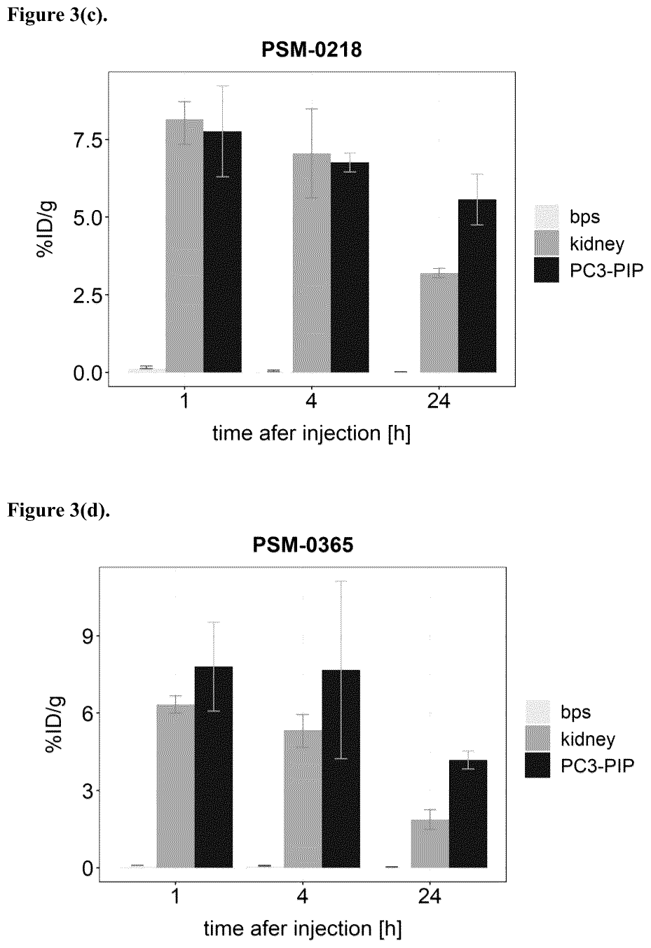

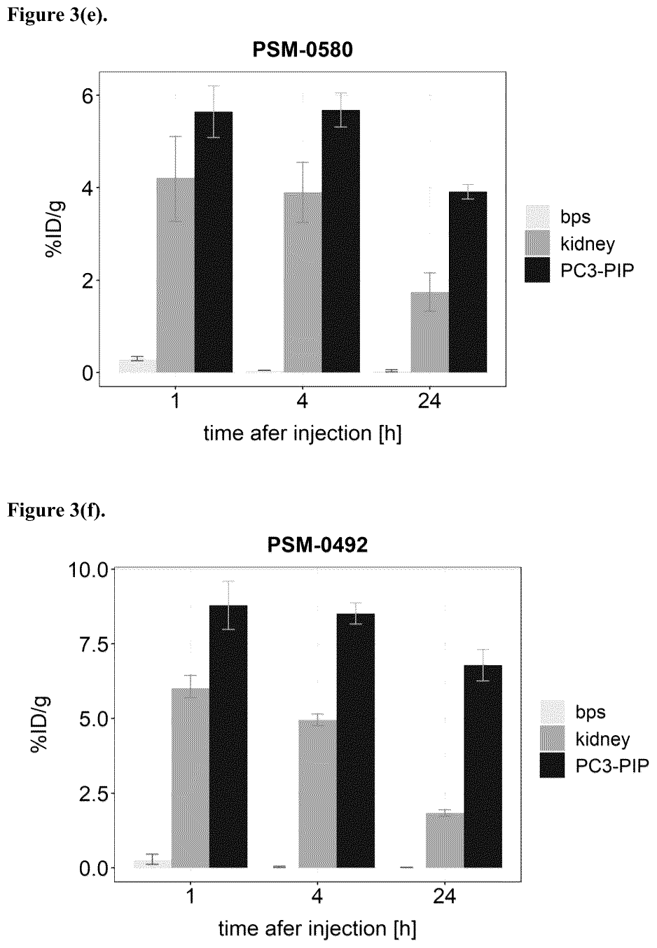

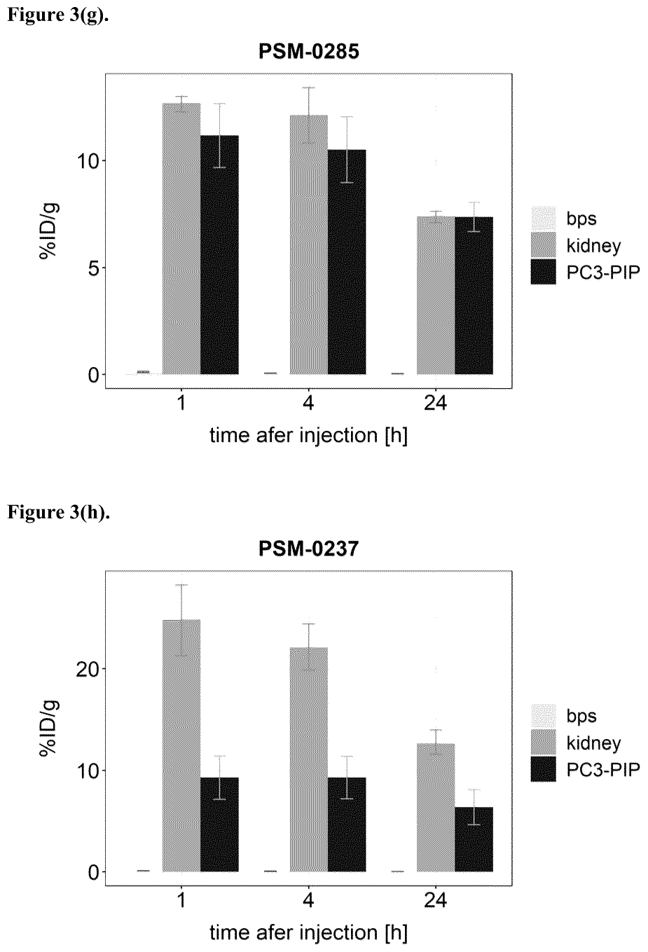

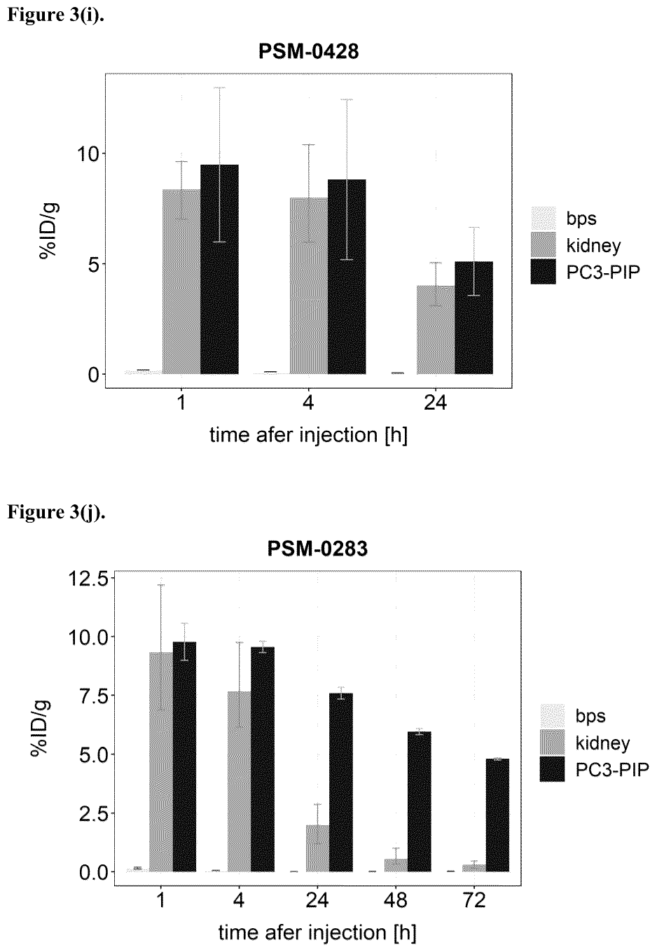

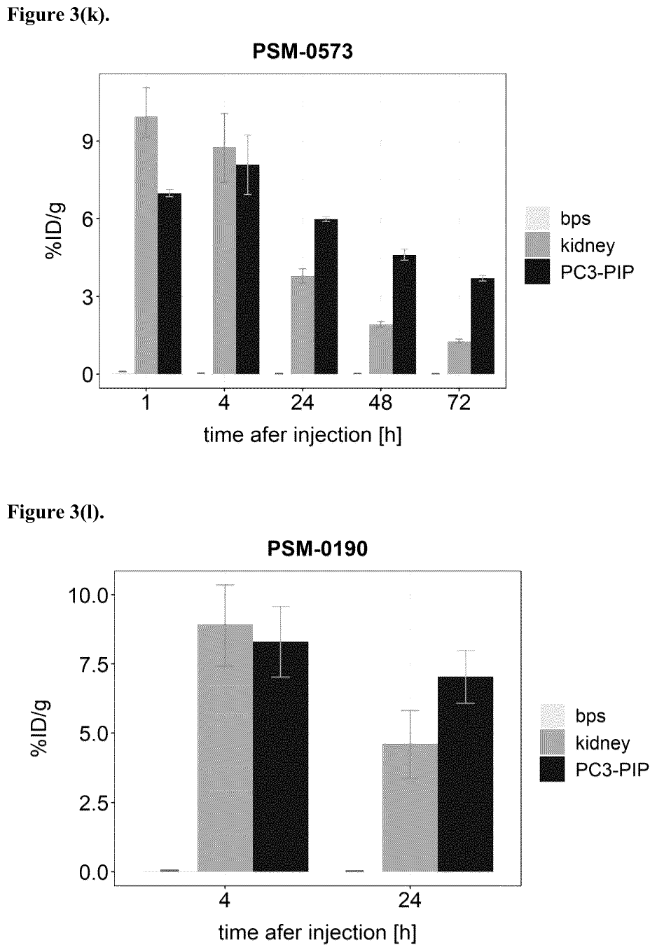

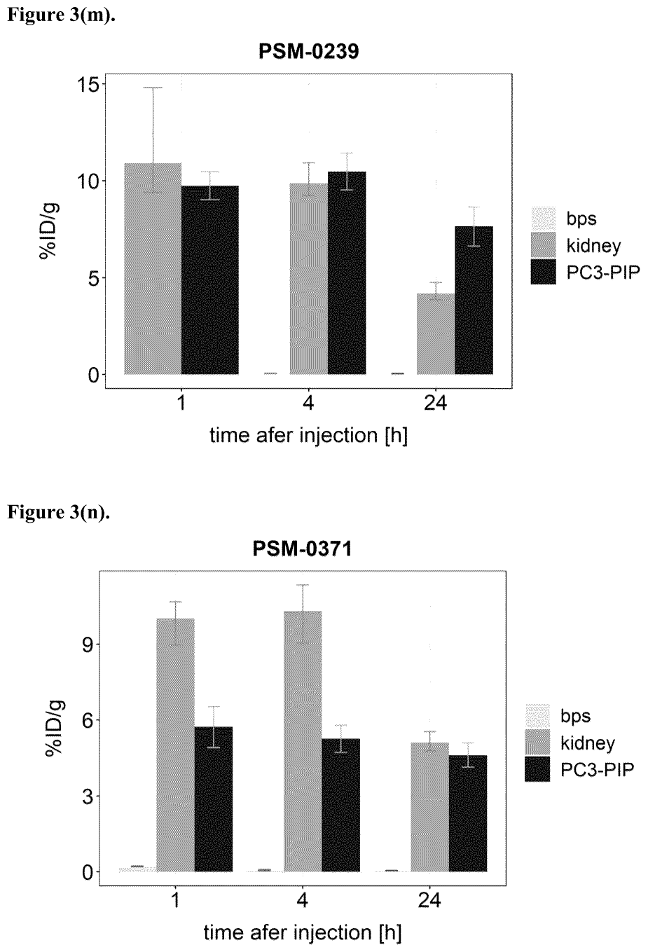

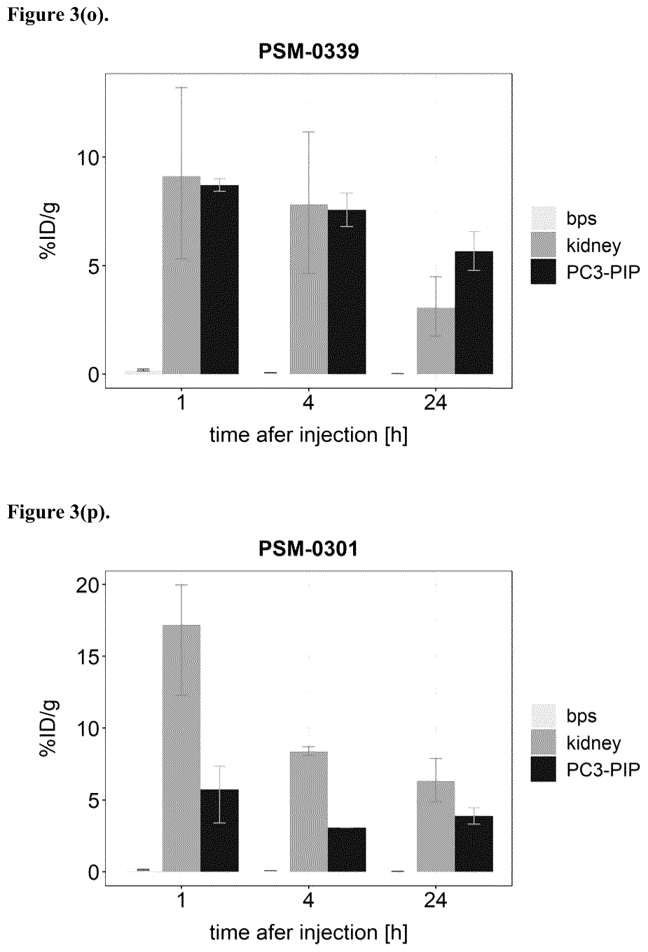

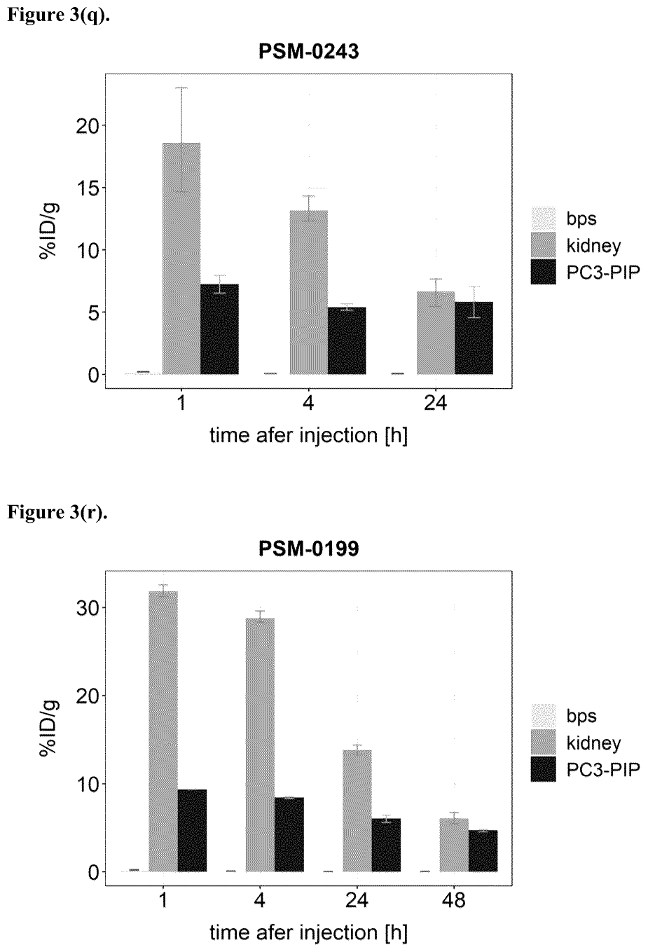

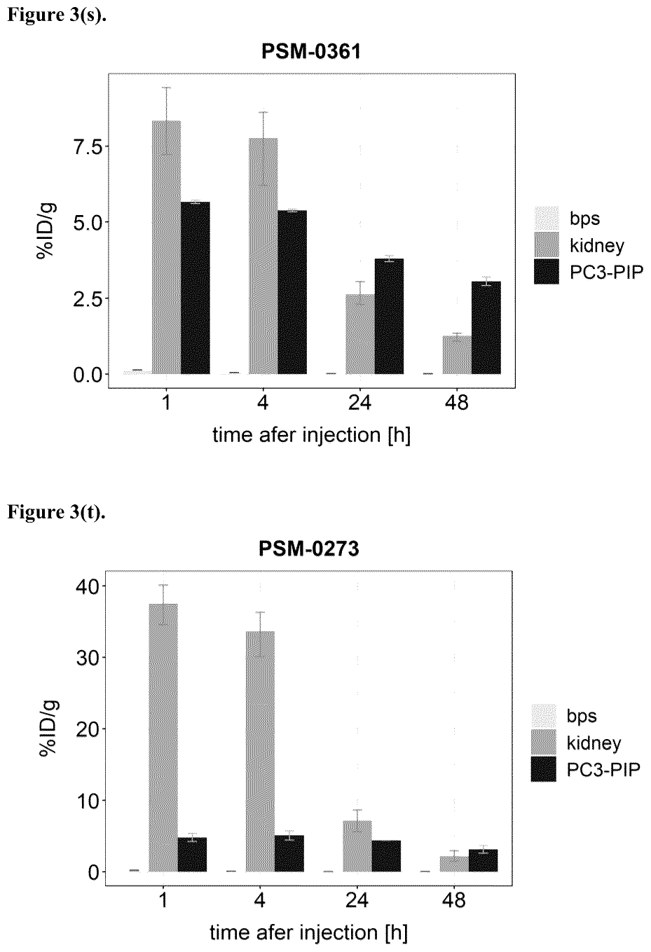

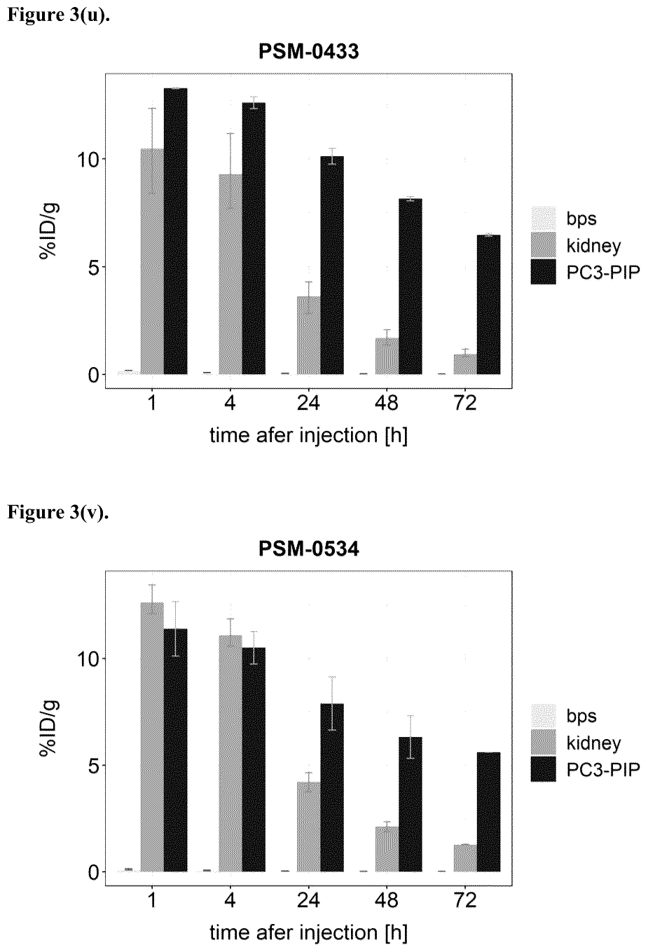

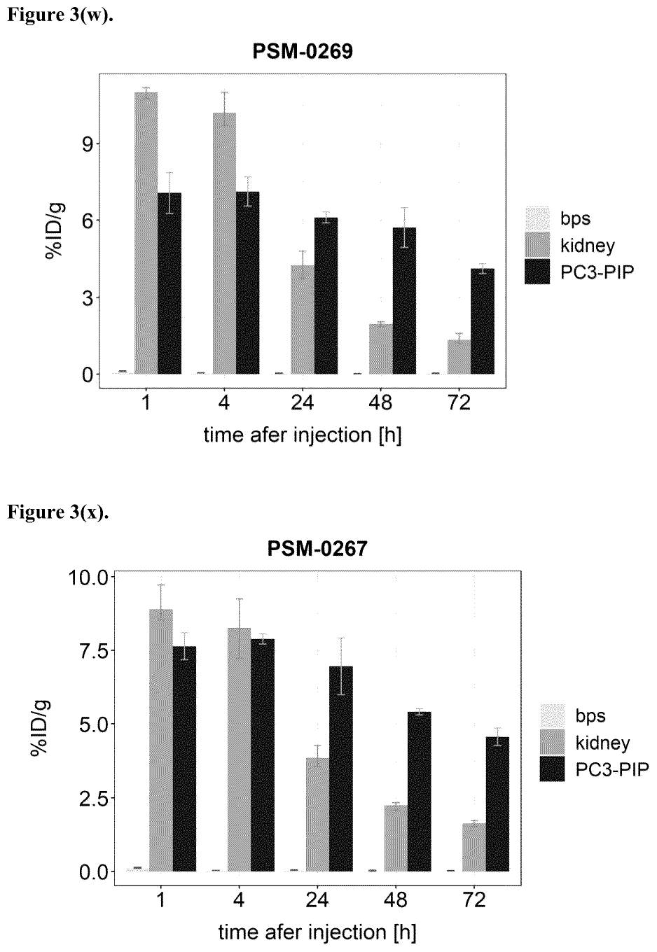

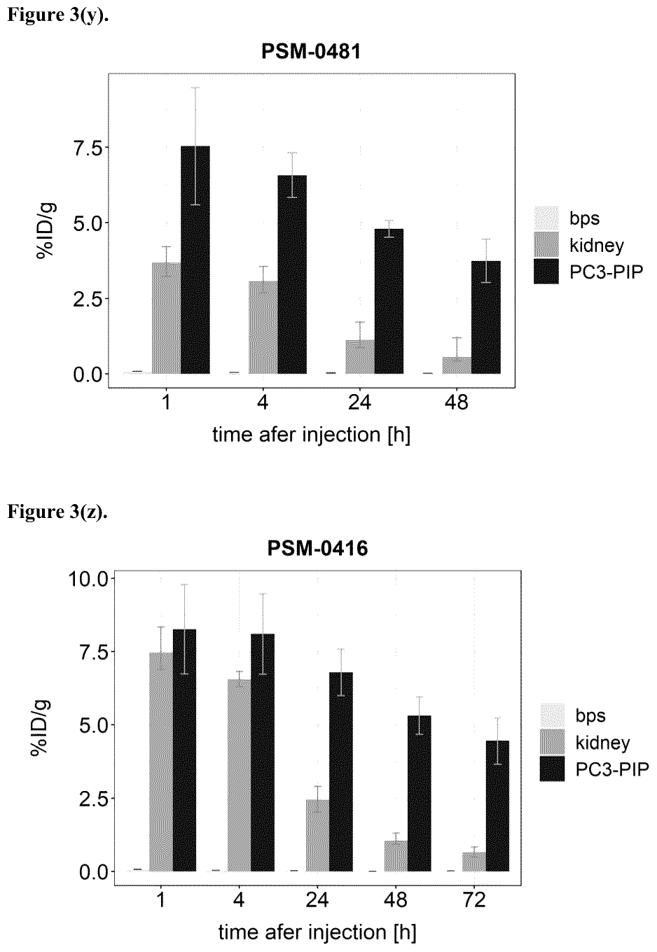

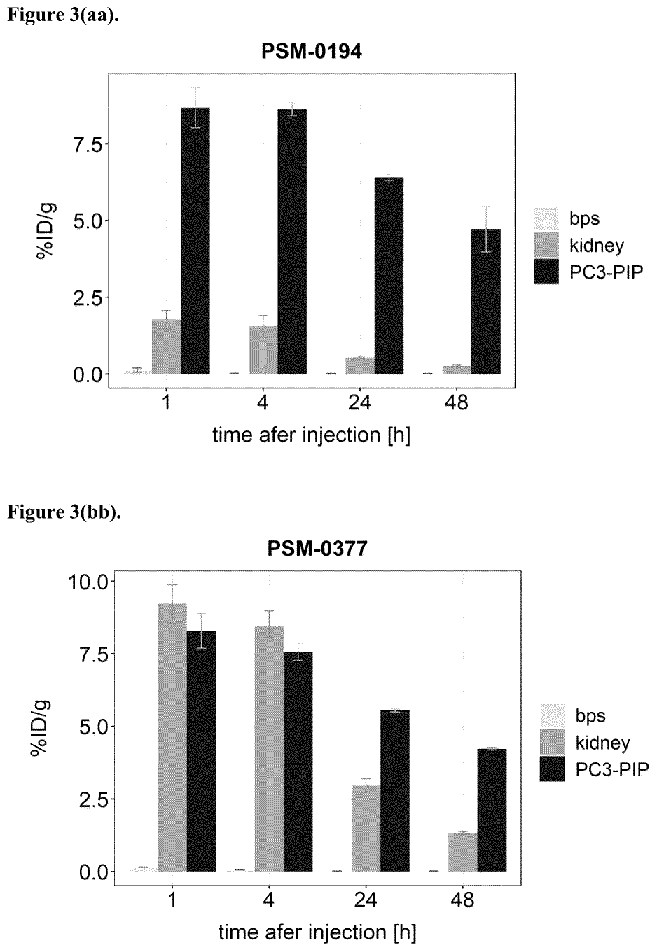

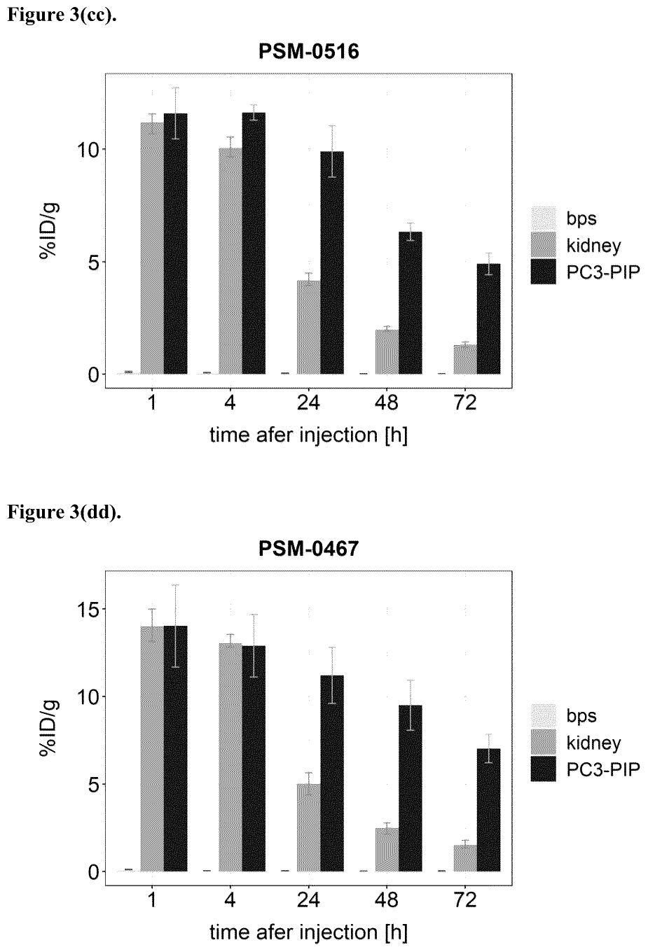

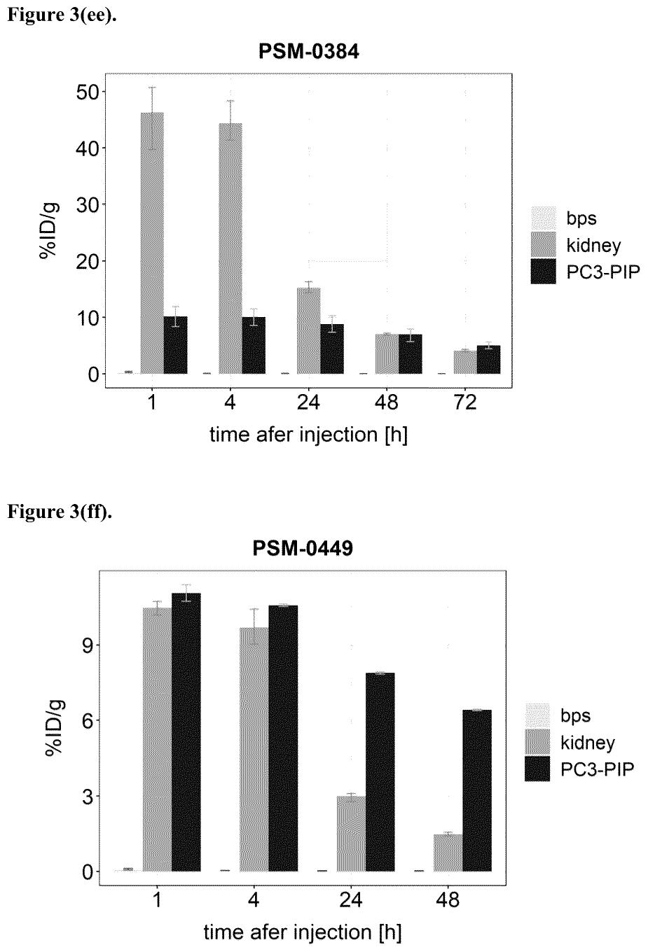

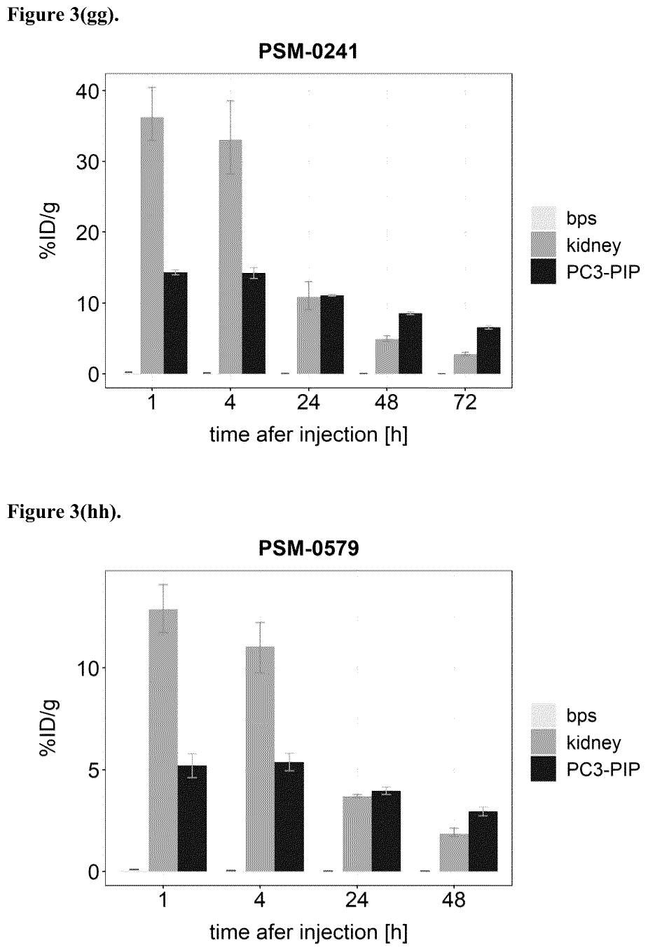

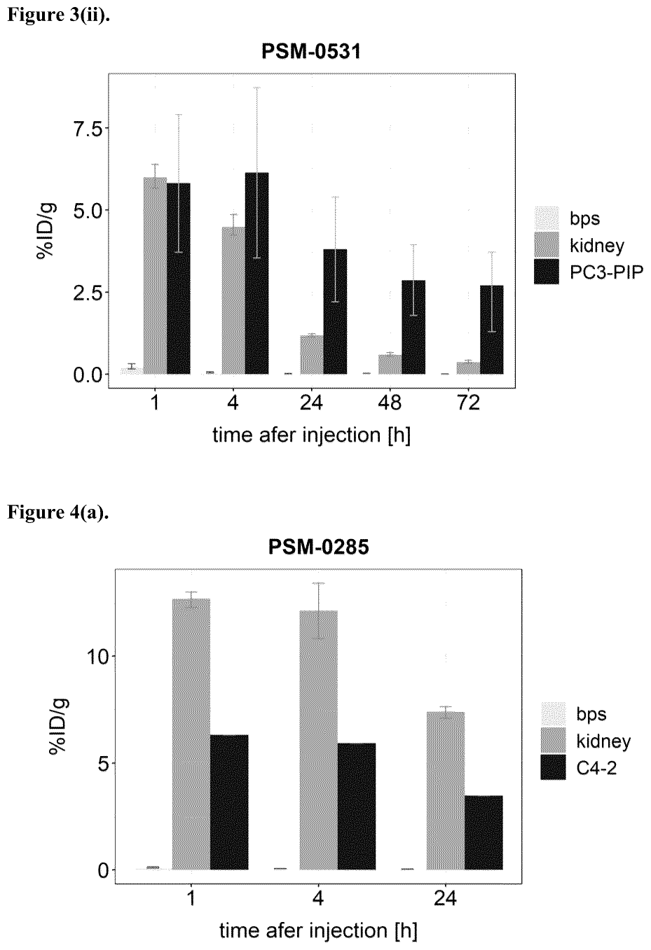

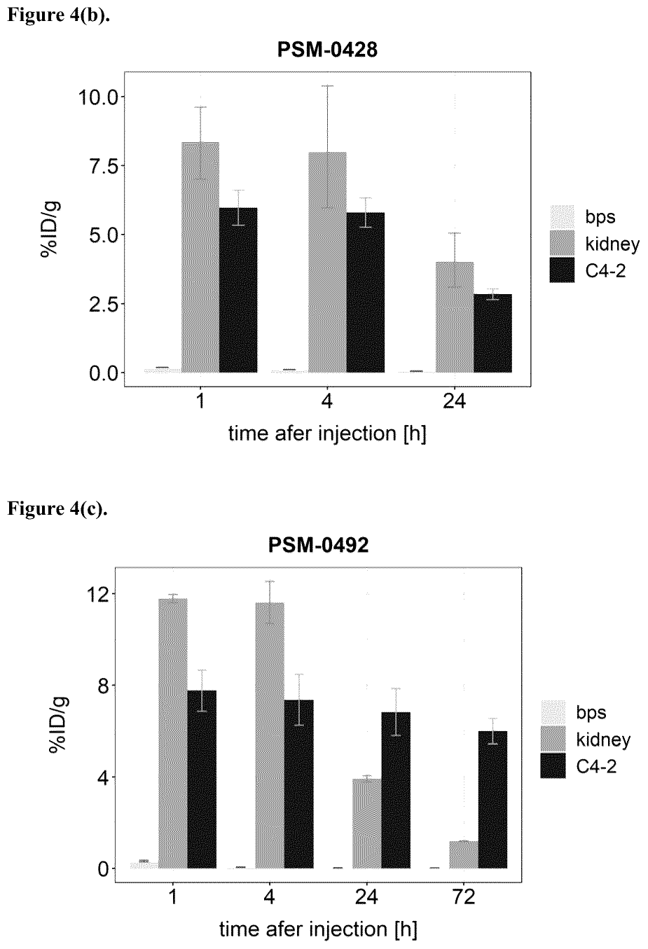

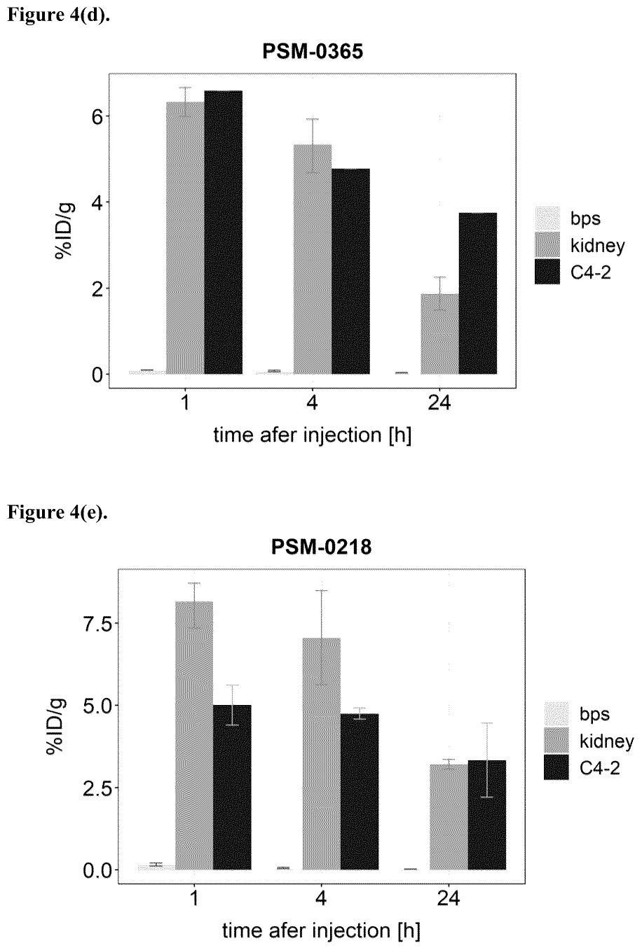

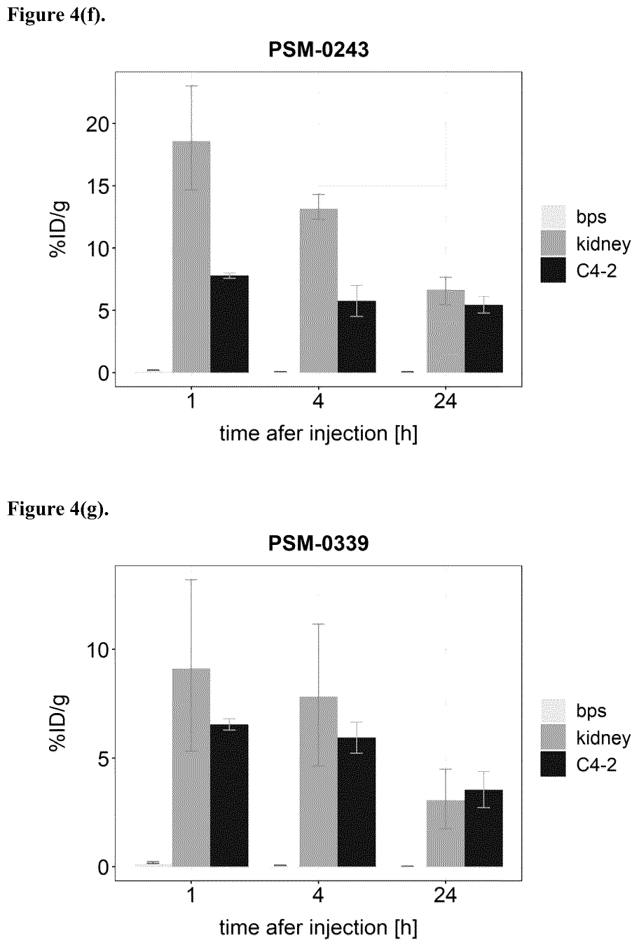

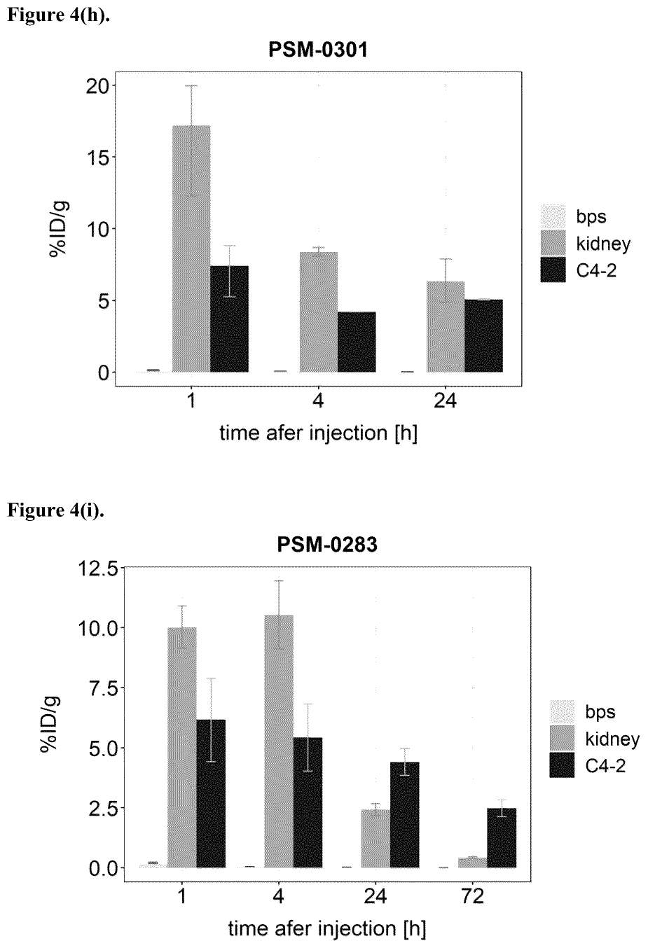

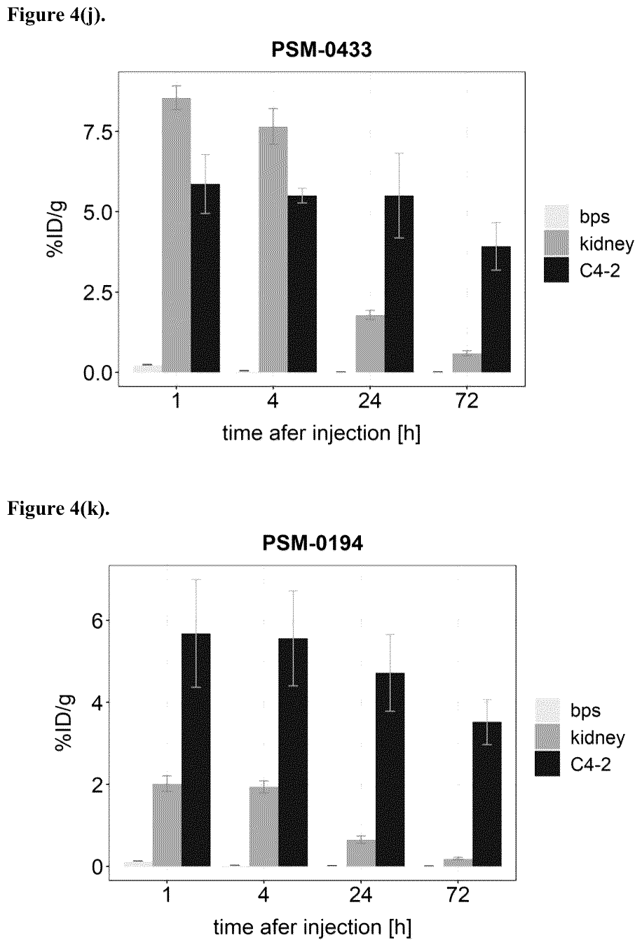

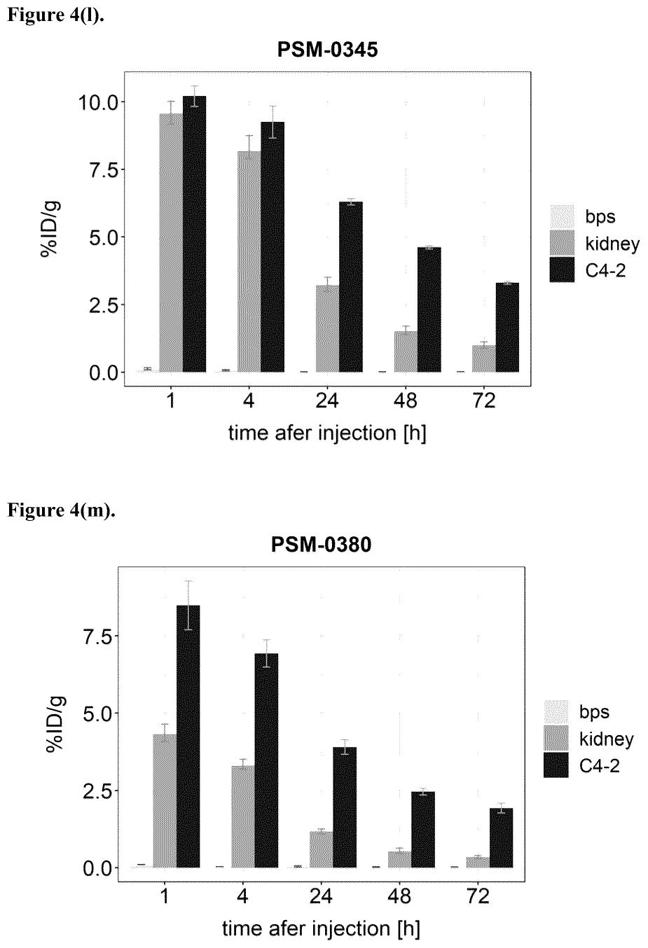

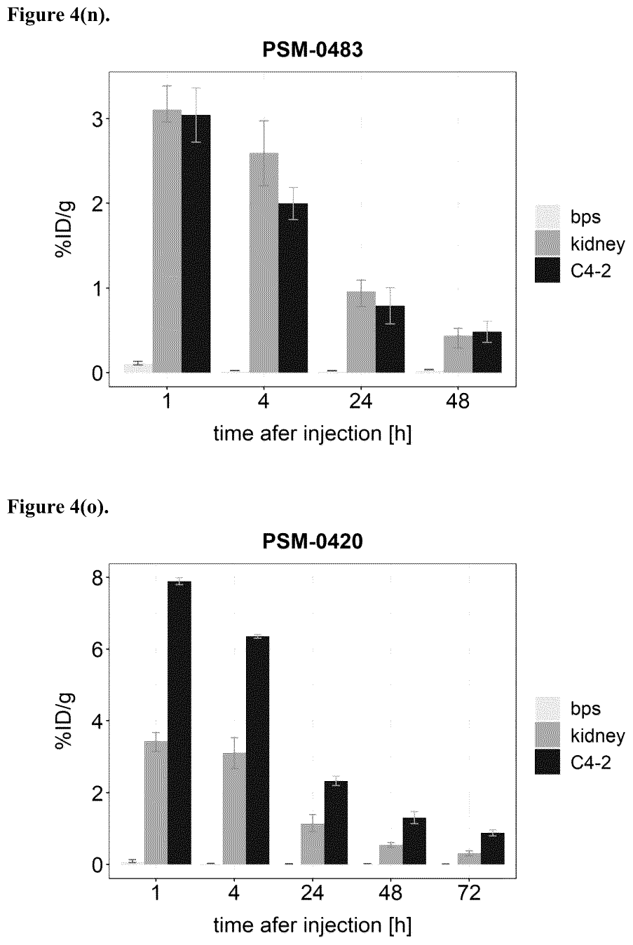

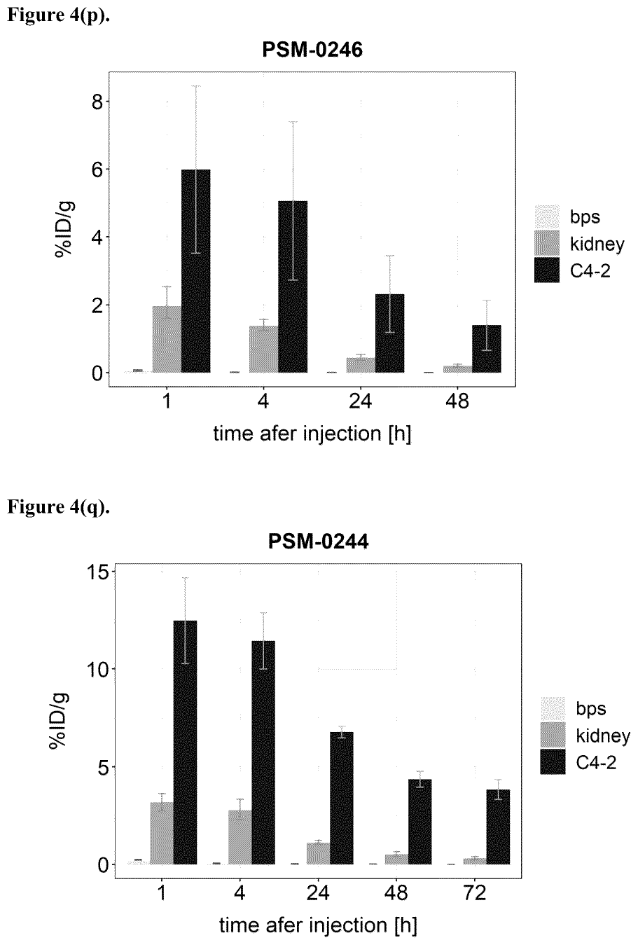

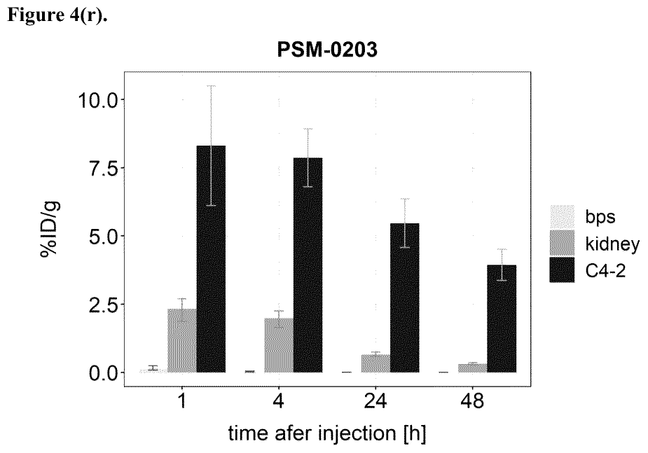

Imaging data were saved as DICOM files and analysed using VivoQuant™ software (Invicro, Boston, USA). In brief, regions of interest (ROIs) were drawn based on computed tomography images for the relevant organs and tissue regions such as heart (estimate of blood pool, bps), kidney, and tumor. The resulting numeric data expressed as a percentage of injected dose per gram of tissue (% ID/g) are presented in FIGS. 3(a)-3(ii) for the PC3-PIP model and FIGS. 4(a)-4(r) for the C4-2 model. Bars show mean % ID/g values of two to three animals per time point. Error bars indicate the value range (minimum and maximum values). Table 11 shows the AUC for tumor uptake in the 1-24 h time span and the AUC ratio (tumor/kidney) for the 1-24 h time span for compounds tested in the PC3-PIP model, and Table 12 shows the AUC for tumor uptake in the 1-24 h time span and the AUC ratio (tumor/kidney) for the 1-24 h time span for compounds tested in the C4-2 model. AUC values were calculated by trapezoidal rule from the mean % ID/g values at 1 h, 4 h and 24 h after injection.

TABLE 11
1-24 h time span Tumor AUC for 1-24 h time span [% ID/g*h] and
AUC tumor-to-kidney ratio [T/K] values in PC3-PIP model

| | | Tumor AUC[% | AUC ratio |
|---|---|---|---|
| Compound ID | Model | ID/g*h] | [T/K] |
| PSM-0194 | PC3-PIP | 180 | 6.7 |
| PSM-0199 | PC3-PIP | 170 | 0.33 |
| PSM-0218 | PC3-PIP | 150 | 1.2 |
| PSM-0234 | PC3-PIP | 62 | 0.64 |
| PSM-0237 | PC3-PIP | 180 | 0.44 |
| PSM-0239 | PC3-PIP | 210 | 1.2 |
| PSM-0241 | PC3-PIP | 300 | 0.54 |
| PSM-0243 | PC3-PIP | 130 | 0.53 |
| PSM-0267 | PC3-PIP | 170 | 1.2 |
| PSM-0269 | PC3-PIP | 150 | 0.87 |
| PSM-0273 | PC3-PIP | 110 | 0.21 |
| PSM-0283 | PC3-PIP | 200 | 1.6 |
| PSM-0285 | PC3-PIP | 210 | 0.91 |
| PSM-0301 | PC3-PIP | 83 | 0.45 |
| PSM-0339 | PC3-PIP | 160 | 1.2 |
| PSM-0361 | PC3-PIP | 110 | 0.85 |
| PSM-0365 | PC3-PIP | 140 | 1.6 |
| PSM-0371 | PC3-PIP | 120 | 0.62 |
| PSM-0377 | PC3-PIP | 160 | 1.1 |
| PSM-0384 | PC3-PIP | 220 | 0.30 |
| PSM-0416 | PC3-PIP | 170 | 1.6 |
| PSM-0425 | PC3-PIP | 230 | 0.53 |
| PSM-0428 | PC3-PIP | 170 | 1.2 |
| PSM-0433 | PC3-PIP | 270 | 1.7 |
| PSM-0449 | PC3-PIP | 220 | 1.4 |
| PSM-0467 | PC3-PIP | 280 | 1.3 |
| PSM-0481 | PC3-PIP | 130 | 2.6 |
| PSM-0492 | PC3-PIP | 180 | 2.1 |
| PSM-0516 | PC3-PIP | 250 | 1.4 |
| PSM-0531 | PC3-PIP | 120 | 1.6 |
| PSM-0534 | PC3-PIP | 220 | 1.1 |
| PSM-0573 | PC3-PIP | 160 | 1.1 |
| PSM-0579 | PC3-PIP | 110 | 0.6 |
| PSM-0580 | PC3-PIP | 110 | 1.6 |

TABLE 12
1-24 h time span Tumor AUC for 1-24 h time span [% ID/g*h] and
AUC tumor-to-kidney ratio [T/K] values in C4-2 model

| | | Tumor AUC | AUC ratio |
|---|---|---|---|
| Compound ID | Model | [% ID/g*h] | [T/K] |
| PSM-0194 | C4-2 | 180 | 4.5 |
| PSM-0203 | C4-2 | 160 | 4.8 |
| PSM-0218 | C4-2 | 95 | 0.76 |
| PSM-0243 | C4-2 | 130 | 0.54 |
| PSM-0244 | C4-2 | 220 | 4.5 |
| PSM-0246 | C4-2 | 90 | 3.9 |
| PSM-0283 | C4-2 | 120 | 0.72 |
| PSM-0285 | C4-2 | 110 | 0.48 |
| PSM-0301 | C4-2 | 110 | 0.59 |
| PSM-0339 | C4-2 | 110 | 0.84 |
| PSM-0345 | C4-2 | 180 | 1.3 |
| PSM-0365 | C4-2 | 100 | 1.1 |
| PSM-0380 | C4-2 | 130 | 2.3 |
| PSM-0420 | C4-2 | 110 | 2.1 |
| PSM-0428 | C4-2 | 100 | 0.72 |
| PSM-0433 | C4-2 | 130 | 1.1 |
| PSM-0483 | C4-2 | 35 | 0.80 |
| PSM-0492 | C4-2 | 160 | 0.86 |

### Example 36: Efficacy Study in Mice with PSMA-Expressing ST1273 Tumors

The efficacy of ¹⁷⁷Lu-PSM-0194 (PSM-0194 labeled with radioactive Lutetium-177) was investigated in the PDX tumor model ST1273. This model of a human PMSA-expressing prostate adenocarcinoma demonstrates uptake and efficacy of ¹⁷⁷Lu-PSM-0194. The ST1273 PDX model was developed at XenoSTART (San Antonio, Texas, USA) and PSMA expression was confirmed by immunohistochemistry. Specific tumor uptake of ¹⁷⁷Lu-PSM-0194 in ST1273 tumor bearing mice was demonstrated by SPECT/computed tomography imaging.

ST1273 tumor fragments were transplanted subcutaneously at the right flank of female NMRI nu/nu mice. In brief, the parent animals with ST1273 PDX tumors for transplantation were euthanized by cervical dislocation. The tumors were resected and trimmed to remove the connective tissues. The tumors were put in a Petri dish with PBS and cut with a scalpel into pieces of approximately 5×5×5 mm. Recipient mice were anesthetized (isoflurane, 1-3% in ambient air supplemented with 100% O₂) and an incision cut in the skin on the back. Room was made for the tumor between the muscle and skin with forceps. The tumor pieces were dipped in PBS before being placed under the skin with forceps. The incision was closed with a 7 mm wound clip. Another incision was cut in the skin on the back, where a testosterone rod was inserted subcutaneously with forceps (PreclinApps, Testosterone MedRod 75 μg/day, drug release duration 100 days). The incision was closed with non-absorbable prolene 5-0 sutures and animals were chipped for identification and allowed to recover from anesthesia. Tumor growth and animal weight were measured twice a week starting 7 days after inoculation. Tumor size was measured by caliper and the volume was estimated using the following formula 0.52×(length×width²).

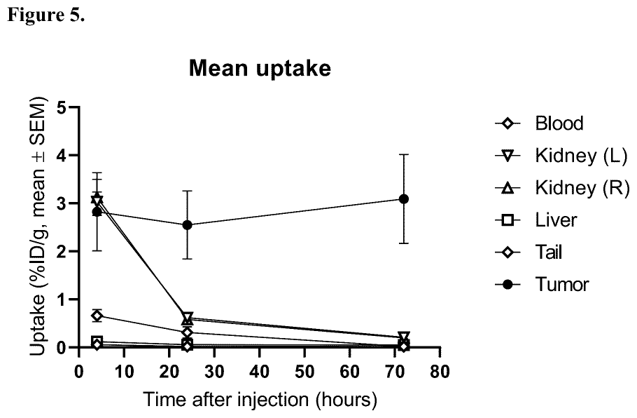

6-7 weeks after inoculation, three mice with ST1273 PDX tumors were dosed with ¹⁷⁷Lu-PSM-0194 and were subjected to SPECT/computed tomography scans. Tumor size measurement was continued for 42 days after dosing. In brief, PSM-0194 was labeled with radioactive Lutetium-177 with a molar activity of 30 MBq/nmol. An activity dose of 30 MBq per mouse was injected intravenously. The mice injected with ¹⁷⁷Lu—PSM-0194 underwent SPECT/computed tomography scans at 4, 24, and 72 hours p.i. to evaluate the distribution of ¹⁷⁷Lu-PSM-0194 in different tissues. Acquisition and reconstruction parameters of SPECT/computed tomography imaging are summarized in Table 13. Regions of interest (ROI) were drawn based on computed tomography images for the tumor and following organs: heart (estimate of blood), kidney (left), kidney (right), and tail. Uptake of ¹⁷⁷Lu-PSM-0194 (% ID/g, decay-corrected) in the corresponding ROI was determined by quantitative imaging analysis. FIG. 5 shows the observed in vivo biodistribution of ¹⁷⁷Lu—PSM-0194 over time (% ID/g, decay-corrected). The mean AUC for the timespan from 4 h to 72 h for the tumor was 190% ID/g*h. Highest uptake in non-tumor organs was found in the kidneys with a favorable tumor-to-kidney ratio of 3.3-3.4. AUC values for the timespan from 4 h to 72 h and the AUC-based tumor-to-organ ratios are summarized in Table 14. SPECT/computed tomography imaging confirmed the high specific tumor uptake of ¹⁷⁷Lu—PSM-0194 in the PDX model ST1273.

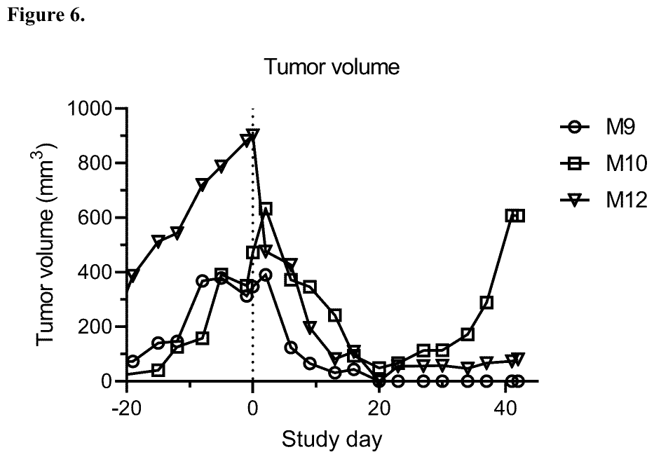

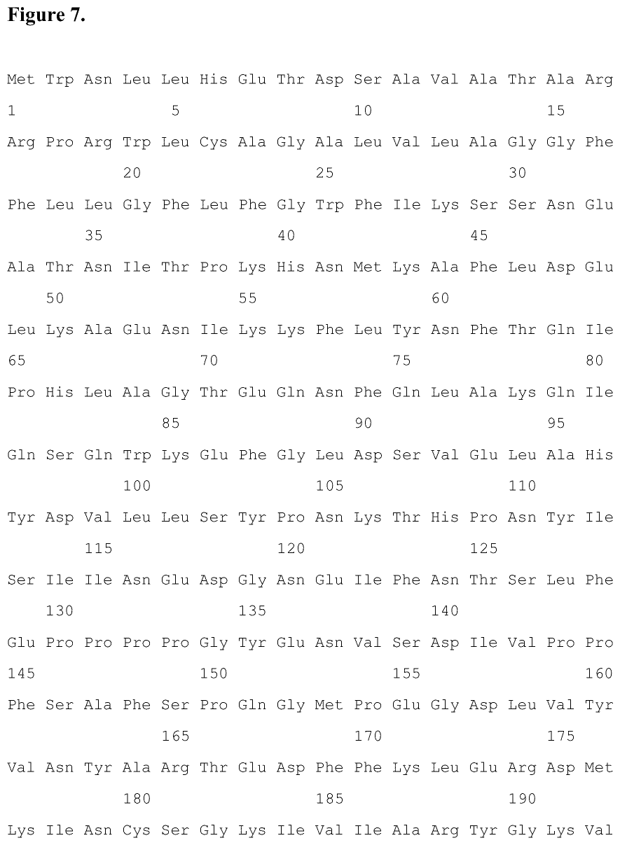

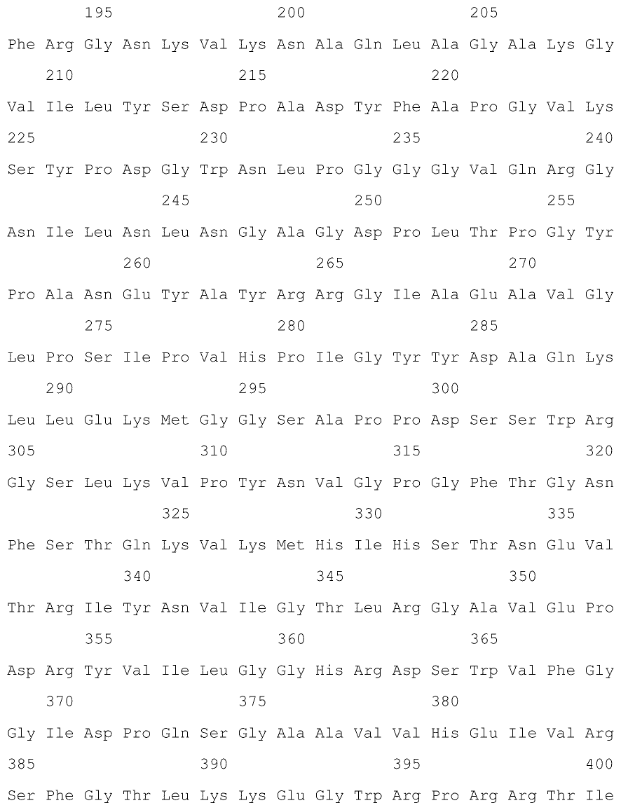

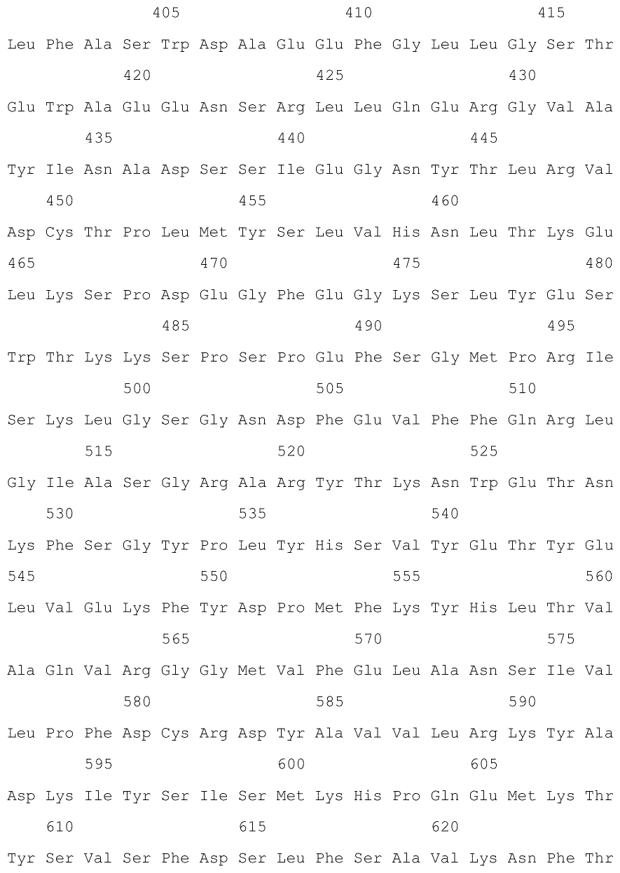

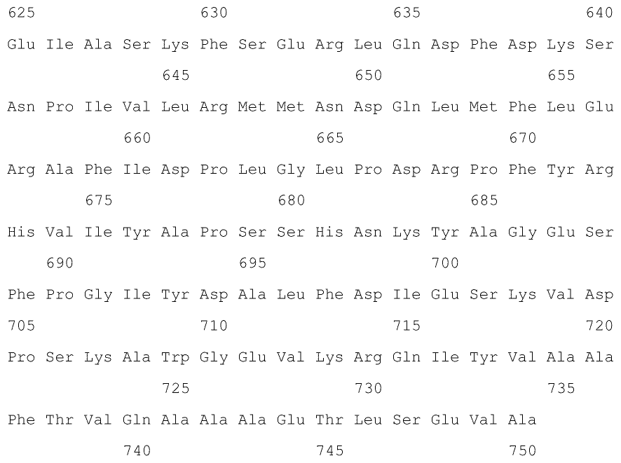

The tumor size measurements were continued for 42 days after dosing. The effect of therapy ¹⁷⁷Lu—PSM-0194 on the tumor volume was evaluated for each individual mouse. FIG. 6 shows the tumor volumes over time. A reduction of the tumor volumes after treatment with ¹⁷⁷Lu—PSM-0194 was observed in all mice. Tumor volume continued declining to a nadir on day 20, with a mean tumor volume (MTV) of 16±16 mm³ (mean SEM). Animal M10 showed re-growth of the tumor starting on day 23 and on day 42 tumor volume was 407 mm³, while the tumors for the other two mice remained suppressed. Tumor volumes (mm³) and relative tumor volumes (%) for each treated mouse are presented in Table 15. For the calculation of relative volumes, the tumor volumes on the day of dosing (day 0) were set to 100%.

The strong tumor regression and sustained tumor suppression after treatment with ¹⁷⁷Lu—PSM-0194 in a PSMA-expressing PDX mouse model confirms the therapeutic potential.

TABLE 13
Acquisition and reconstruction parameters
of SPECT/computed tomography imaging
SPECT Parameters

| Animals per bed | 3 | | |
|---|---|---|---|
| Type of SPECT scanning | Multi-pinhole | | |
| | Peaks (keV) | | Full width (%) |
| Energy Window(s) | Primary | 208.4 | 20% |
| | Secondary | 112.9 | 20% |
| | Tertiary | 56.1 | 20% |

| | |
|---|---|
| SPECT acquisition start | At 4, 24 and 72 hours after injection |
| Reconstruction | Tera Tomo 3D reconstruction with |
| | attenuation and scatter correction |

CT Parameters

| | |
|---|---|
| Animals per bed | 3 |
| Type of scan | Helical |
| Projections | 480 |
| Pitch | 1 |
| X-ray power (kVp) | 50 |
| Exposure time (ms) | 300 |
| Reconstruction | 250 |
| resolution (μm) | |
| Binning | 1:4 |

TABLE 14
4 h to 72 h time span AUC for the 4 h to 72 h time
span [% ID/g*h] and tumor-to-organ ratio values in
of ¹⁷⁷Lu- PSM-0194 in the ST1273 tumor model

| | Organ/ROI | | | | | |
|---|---|---|---|---|---|---|
| | | Kidney | Kidney | | | |
| | Blood | (left) | (right) | Liver | Tail | Tumor |
| AUC 4-72 h (mean | 1.6 | 56 | 56 | 4.2 | 18 | 190 |
| uptake, % ID/g*h) | | | | | | |
| Tumor-to-organ | 115 | 3.3 | 3.4 | 45 | 11 | NA |
| ratio | | | | | | |

TABLE 15
Tumor volumes (mm³) and relative tumor volumes (%)

| | Tumor volumes (mm³) | | | Relative tumor volume (%) | | |
|---|---|---|---|---|---|---|
| Study Day | M9 | M10 | M12 | M9 | M10 | M12 |
| −40 | 0.0 | 0.0 | 0.0 | 0.0 | 0.0 | 0.0 |
| −35 | 26 | 0.0 | 31 | 7.4 | 0.0 | 3.5 |
| −32 | 0.0 | 0.0 | 26 | 0.0 | 0.0 | 2.9 |
| −29 | 0.0 | 0.0 | 43 | 0.0 | 0.0 | 4.8 |
| −26 | 0.0 | 0.0 | 79 | 0.0 | 0.0 | 8.7 |
| −22 | 72 | 19 | 209 | 21 | 4.1 | 23 |
| −19 | 73 | ND | 387 | 21 | 0.0 | 43 |
| −15 | 140 | 40 | 512 | 40 | 8.6 | 57 |
| −12 | 147 | 126 | 543 | 42 | 27 | 60 |
| −8 | 368 | 158 | 720 | 106 | 34 | 80 |
| −5 | 379 | 391 | 788 | 109 | 83 | 87 |
| −1 | 313 | 350 | 882 | 90 | 74 | 98 |
| 0 | 348 | 472 | 902 | 100 | 100 | 100 |
| 2 | 390 | 633 | 476 | 112 | 134 | 53 |
| 6 | 124 | 372 | 427 | 36 | 79 | 47 |
| 9 | 65 | 346 | 196 | 19 | 73 | 23 |
| 13 | 31 | 242 | 82 | 9.0 | 51 | 9.1 |
| 16 | 43 | 94 | 109 | 12 | 20 | 12 |
| 20 | 0 | 49 | 0 | 0.0 | 10 | 0.0 |
| 23 | 0 | 66 | 55 | 0.0 | 14 | 6.1 |
| 27 | 0 | 113 | 56 | 0.0 | 24 | 6.2 |
| 30 | 0 | 114 | 58 | 0.0 | 24 | 6.4 |
| 34 | 0 | 172 | 47 | 0.0 | 36 | 5.2 |
| 37 | 0 | 289 | 67 | 0.0 | 61 | 7.4 |
| 41 | 0 | 608 | 74 | 0.0 | 129 | 8.2 |
| 42 | 0 | 607 | 81 | 0.0 | 129 | 9.0 |

### REFERENCES

The disclosure of each and any document recited herein is incorporated by reference.

1. A compound of Formula (I), or a pharmaceutically acceptable salt, a pharmaceutically acceptable solvate or a hydrate thereof, 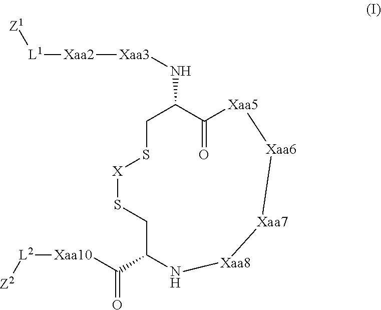
wherein
X is selected from the group consisting of bond and —CH₂—;
Z¹ is selected from the group consisting of chelator and NT;
NT is selected from the group consisting of H, Ac, Hex, HPA, HO-Succinyl, SaPr, Iva, HYDAc, Bio, nBuCAyl, AF488Ahx, and Hib;
L¹ is selected from the group consisting of a bond and -(Xaa1)ₖ-,
k is selected from the group consisting of 1, 2, and 3,
Xaa1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI), 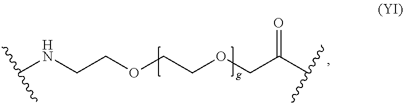
g is an integer selected from the group consisting of 0-23,
wherein,
if k=1, Xaa1 is covalently bound to Z¹ and covalently bound to Xaa2,
if k=2, a first of the two Xaa1 is covalently bound to Z¹ and covalently bound to a second of the two Xaa1, and the second of the two Xaa1 is covalently bound to the first of the two Xaa1 and covalently bound to Xaa2,
if k=3, a first of the three Xaa1 is covalently bound to Z¹ and covalently bound to a second of the three Xaa1, the second of the three Xaa1 is covalently bound to the first of the three Xaa1 and covalently bound to a third of the three Xaa1, and the third of the three Xaa1 is covalently bound to the second of the three Xaa1 and covalently bound to Xaa2;
wherein when L¹ is bond, then Z¹ is NT;
Xaa2 is selected from the group consisting of Formula (II) and Formula (III): 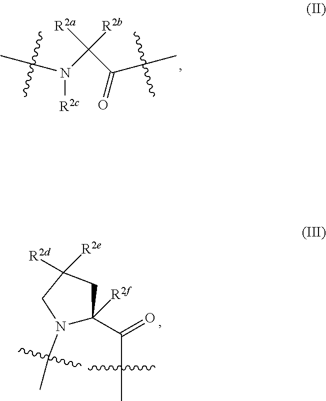
wherein R^{2a} is selected from the group consisting of H, (C₁-C₆)alkyl, and CH₂R^{2g};
R^{2g} is selected from the group consisting of OH, and CO₂H;
R^{2b} is selected from the group consisting of H and (C₁-C₆)alkyl;
or alternatively, R^{2a} and R^{2b} can together form a 5 or 6 membered carbocycle or heterocycle,
R^{2c} is selected from the group consisting of H and CH₃;
R^{2d} is selected from the group consisting of H, F, and OH;
R^{2e} is selected from the group consisting of H and F;
R^{2f} is selected from the group consisting of H and CH₃;
with the proviso that Xaa2 can be absent when L¹ is bond and NT is Hib;
Xaa3 is selected from Formula (IV): 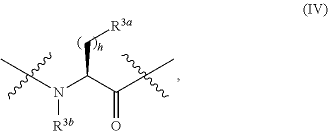
wherein R^{3a} is selected from the group consisting of aryl, (C₅-C₆)heteroaryl, indol-3-yl, 6-chloro-1H-indol-3-yl, and —S—CH₂-phenyl; and wherein said aryl or said heteroaryl ring of R^{3a} is optionally substituted by 1, 2, or 3 substituents independently selected from the group consisting of halogen, (C₁-C₆)alkyl, CN, OH, and —O(C₁-C₃)alkyl, wherein said (C₁-C₆)alkyl may optionally be substituted by one or more fluorine;
R^{3b} is selected from the group consisting of H and CH₃;
h is selected from the group consisting of 1 and 2;
Xaa5 is selected from the group consisting of Formula (Va), Formula (Vb), Hgn, lys, Nle, Tap, Aph, and Gln: 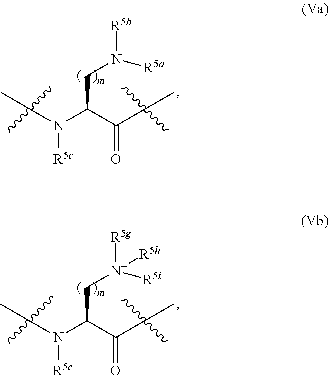
wherein R^{5a} is selected from the group consisting of H, (C₁-C₆)alkyl, Ac, C(═NR^{5d})NR^{5e}R^{5f}, and Bio;
R^{5d} is selected from the group consisting of H and CH₃;
R^{5e} and R^{5f} are independently selected from the group consisting of H and (C₁-C₆)alkyl;
R^{5b} is selected from the group consisting of H and (C₁-C₆)alkyl;
R^{5c} is selected from the group consisting of H and CH₃;
m is selected from the group consisting of 2, 3, 4, and 5; and
R^{5g}, R^{5h}, and R⁵ⁱ are independently selected from the group consisting of (C₁-C₆)alkyl;
Xaa6 is selected from the group consisting of Formula (VI), Formula (VII), Nle, and arg: 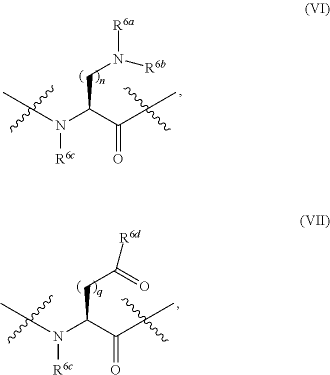
wherein R^{6a} is selected from the group consisting of H, C(═NR^{6e})NR^{6f}R^{6g}, C(═O)R^{6h}, and pyridyl;
R^{6e} is selected from the group consisting of H and CH₃;
R^{6f} is selected from the group consisting of H, (C₁-C₆)alkyl, Ac, NO₂, and C(═O)NR⁶ⁱR^{6j};
R⁶ⁱ and R^{6j} are independently selected from the group consisting of H and (C₁-C₂)alkyl;
R^{6g} is selected from the group consisting of H and (C₁-C₆)alkyl;
or alternatively, R^{6e} and R^{6f} can together form a 5 or 6 membered heterocycle;
R^{6h} is selected from the group consisting of (C₁-C₆)alkyl, NR^{6k}R^{6m}, and NR⁶ⁿC(═NR^{6p})NR^{6q}R^{6r};
R^{6k} and R^{6m} are independently selected from the group consisting of H and (C₁-C₆)alkyl;
R⁶ⁿ and R^{6p} are independently selected from the group consisting of H and CH₃;
R^{6q} and R^{6r} are independently selected from the group consisting of H and (C₁-C₆)alkyl;
R^{6b} is selected from the group consisting of H and (C₁-C₆)alkyl;
R^{6c} is selected from the group consisting of H and CH₃;
n is selected from the group consisting of 1, 2, 3, and 4;
R^{6d} is selected from the group consisting of NR^{6s}C(═NR^{6t})NR^{6u}R^{6v}, OH, and NR^{6w}R^{6x};
R^{6s} and R^{6t} are independently selected from the group consisting of H and CH₃;
R^{6u}, R^{6v}, R^{6w}, and R^{6x} are independently selected from the group consisting of H and (C₁-C₆)alkyl; and
q is selected from the group consisting of 2, 3, and 4;
Xaa7 is an amino acid residue, wherein the amino acid residue is preferably selected from the group consisting of Formula (VIII), Formula (IX), Formula (X), Formula (XI), Dtc, and Oic: 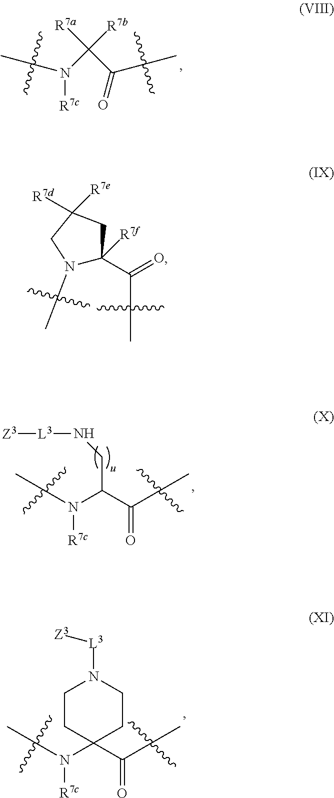
wherein R^{7a} is selected from the group consisting of H, (C₁-C₆)alkyl, and (CH₂)ₜR^{7g};
R^{7g} is selected from the group consisting of OH, CO₂H, and NR^{7h}R⁷ⁱ;
R^{7h} and R⁷ⁱ are independently selected from the group consisting of H and (C₁-C₆)alkyl;
t is selected from the group consisting of 1, 2, 3 and 4;
R^{7b} is selected from the group consisting of H and (C₁-C₆)alkyl;
or alternatively, R^{7a} and R^{7b} can together form a 5 or 6 membered carbocycle or heterocycle,
R^{7c} is selected from the group consisting of H and CH₃;
R^{7d} is selected from the group consisting of H, F, and OH;
R^{7e} is selected from the group consisting of H and F;
R^{7f} is selected from the group consisting of H and CH₃;
u is selected from the group consisting of 2, 3, and 4;
L³ is selected from the group consisting of bond and -(Xab1)ᵥ-,
wherein v is selected from the group consisting of 1, 2, and 3,
Xab1 is each and individually an amino acid residue, preferably the amino acid residue is selected from the group consisting of an α-amino acid residue, a β-amino acid residue, a γ-amino acid residue, a δ-amino acid residue, an ε-amino acid residue, an ω-amino acid residue, and an amino acid residue of Formula (YI),
if v=1, Xab1 is covalently bound to Z³ and covalently bound to a side chain amino function of Formula (X) or of Formula (XI),
if v=2, a first of the two Xab1 is covalently bound to Z³ and covalently bound to a second of the two Xab1, and the second of the two Xab1 is covalently bound to the first of the two Xab1 and covalently bound to a side chain amino function of Formula (X) or of Formula (XI),
if v=3, a first of the three Xab1 is covalently bound to Z³ and covalently bound to a second of the three Xab1, the second of the three Xab1 is covalently bound to the first of the three Xab1 and covalently bound to a third of the three Xab1, and the third of the three Xab1 is covalently bound to the second of the three Xab1 and covalently bound to a side chain amino function of Formula (X) or of Formula (XI);
Z³ is selected from the group consisting of H and chelator;
Xaa8 is an α-amino acid residue, wherein the α-nitrogen atom of Xaa8 is unsubstituted or is optionally substituted by CH₃;
Xaa10 is Formula (XII): 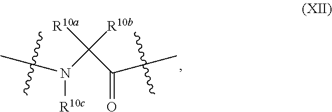
wherein:
R^{10a} is selected from the group consisting of (C₁-C₆)alkyl;
R^{10b} is selected from the group consisting of H and (C₁-C₆)alkyl;
or alternatively, R^{10a} and R^{10b} can together form a 5 or 6 membered carbocycle or heterocycle,
R^{10c} is selected from the group consisting of H and CH₃;
L² is selected from the group consisting of:
bond and -Xaa11-(Xaa12)ₛ-, such that Xaa11 is covalently bound to Xaa10;
Xaa11 is an amino acid residue, wherein the amino acid residue is preferably selected from the group consisting of an α-amino acid residue, a β-amino acid residue, a γ-amino acid residue, a δ-amino acid residue, an ε-amino acid residue, an ω-amino acid residue, and an amino acid residue of Formula (YI);
s is selected from the group consisting of 0, 1, 2, 3, 4, and 5; and
Xaa12 is each and individually an amino acid residue, wherein the amino acid residue is preferably selected from the group comprising an α-amino acid residue, a β-amino acid residue, a γ-amino acid residue, a δ-amino acid residue, an ε-amino acid residue, an ω-amino acid residue, and an amino acid residue of Formula (YI),
if s=0, Xaa11 is covalently bound to Z²,
if s=1, Xaa12 is covalently bound to Xaa11 and covalently bound to Z²,
if s=2, a first of the two Xaa12 is covalently bound to Xaa11 and covalently bound to a second of the two Xaa12, and the second of the two Xaa12 is covalently bound to the first of the two Xaa12 and covalently bound to Z²,
if s=3, a first of the three Xaa12 is covalently bound to Xaa11 and covalently bound to a second of the three Xaa12, the second of the three Xaa12 is covalently bound to the first of the three Xaa12 and covalently bound to a third of the three Xaa12, and the third of the three Xaa12 is covalently bound to the second of the three Xaa12 and covalently bound to Z²,
if s=4, a first of the four Xaa12 is covalently bound to Xaa11 and covalently bound to a second of the four Xaa12, the second of the four Xaa12 is covalently bound to the first of the four Xaa12 and covalently bound to a third of the four Xaa12, the third of the four Xaa12 is covalently bound to the second of the four Xaa12 and covalently bound to a fourth of the four Xaa12, and the fourth of the four Xaa12 is covalently bound to the third of the four Xaa12 and covalently bound to Z²,
if s=5, a first of the five Xaa12 is covalently bound to Xaa11 and covalently bound to a second of the five Xaa12, the second of the five Xaa12 is covalently bound to the first of the five Xaa12 and covalently bound to a third of the five Xaa12, the third of the five Xaa12 is covalently bound to the second of the five Xaa12 and covalently bound to a fourth of the five Xaa12, the fourth of the five Xaa12 is covalently bound to the third of the five Xaa12 and covalently bound to a fifth of the five Xaa12, and the fifth of the five Xaa12 is covalently bound to the fourth of the five Xaa12 and covalently bound to Z²;
Z² is selected from the group consisting of CT, XDa-chelator and an α-amino acid residue of Formula (CT-I): 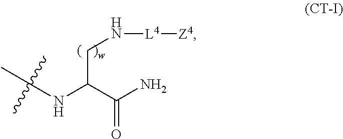
CT is selected from the group consisting of Formula (CT-II), Formula (CT-III), AF488N3K-NH₂, OH, and Throl-OH: 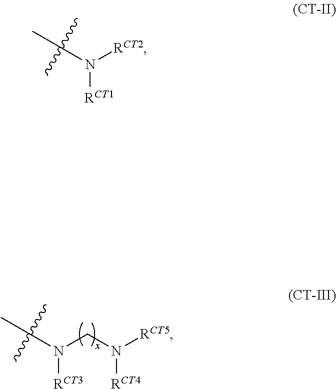
wherein:
R^{CT1} is selected from the group consisting of H and CH₃;
R^{CT2} is selected from the group consisting of H and (C₁-C₆)alkyl;
R^{CT3} and R^{CT4} are each and individually selected from the group consisting of H and CH₃;
R^{CT5} is selected from the group consisting of H and (C₁-C₆)alkyl;
x is selected from the group consisting of 2-10;
XDa is a diamine, wherein said diamine is preferably selected from the group consisting of en and Ape;
w is selected from the group consisting of 1, 2, 3, 4, 5, and 6;
L⁴ is selected from the group consisting of a bond and -(Xac1)_{y}-,
wherein:
y is selected from the group consisting of 0, 1, 2, and 3,
Xac1 is each and individually an amino acid residue, preferably the amino acid residue is selected from the group comprising an α-amino acid residue, a β-amino acid residue, a γ-amino acid residue, a δ-amino acid residue, an ε-amino acid residue, an ω-amino acid residue and an amino acid residue of Formula (YI),
if y=0, the side chain amino function of the α-amino acid residue of Formula (CT-I) is covalently bound to Z⁴,
if y=1, Xac1 is covalently bound to the side chain amino function of the α-amino acid residue of Formula (CT-I) and covalently bound to Z⁴,
if y=2, a first of the two Xac1 is covalently bound to the side chain amino function of the α-amino acid residue of Formula (CT-I) and covalently bound to a second of the two Xac1, and the second of the two Xac1 is covalently bound to the first of the two Xac1 and covalently bound to Z⁴,
if y=3, a first of the three Xac1 is covalently bound to the side chain amino function of the α-amino acid residue of Formula (CT-I) and covalently bound to a second of the three Xac1, the second of the three Xac1 is covalently bound to the first of the three Xac1 and covalently bound to a third of the three Xac1, and the third of the three Xac1 is covalently bound to the second of the three Xac1 and covalently bound to Z⁴,
if y=4, a first of the four Xac1 is covalently bound to the side chain amino function of the α-amino acid residue of Formula (CT-I) and covalently bound to a second of the four Xac1, the second of the four Xac1 is covalently bound to the first of the four Xac1 and covalently bound to a third of the four Xac1, the third of the four Xac1 is covalently bound to the second of the four Xac1 and covalently bound to a fourth of the four Xac1, and the fourth of the four Xac1 is covalently bound to the third of the four Xac1 and covalently bound to Z⁴;
Z⁴ is selected from the group consisting of H and chelator;
with the proviso that if L² is bond and CT is Formula (CT-II) wherein R^{CT1} is selected from the group consisting of H and CH₃ and R^{CT2} is (C₄-C₆)alkyl, then Xaa10 can be absent;
and wherein the compound of Formula (I) may optionally comprise a therapeutically active nuclide or a diagnostically active nuclide.

2. A compound of Formula (I), or a pharmaceutically acceptable salt, a pharmaceutically acceptable solvate or a hydrate thereof, 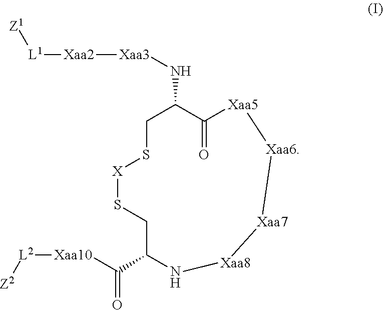
wherein
X is selected from the group consisting of bond and —CH₂—;
Z¹ is selected from the group consisting of chelator and NT;
NT is selected from the group consisting of H, Ac, Hex, HPA, HO-Succinyl, SaPr, Iva, HYDAc, Bio, nBuCAyl, AF488Ahx, and Hib;
L¹ is selected from the group consisting of bond and -(Xaa1)ₖ-;
k is selected from the group consisting of 1, 2, and 3;
wherein,
if k=1, Xaa1 is covalently bound to Z¹ and covalently bound to Xaa2,
if k=2, a first of the two Xaa1 is covalently bound to Z1 and covalently bound to a second of the two Xaa1, and the second of the two Xaa1 is covalently bound to the first of the two Xaa1 and covalently bound to Xaa2,
if k=3, a first of the three Xaa1 is covalently bound to Z1 and covalently bound to a second of the three Xaa1, the second of the three Xaa1 is covalently bound to the first of the three Xaa1 and covalently bound to a third of the three Xaa1, and the third of the three Xaa1 is covalently bound to the second of the three Xaa1 and covalently bound to Xaa2;
wherein when L¹ is bond, then Z¹ is NT;
Xaa1 is each and individually selected from the group consisting of Thr, Ala, Ser, Pamp, Leu, Ile, Nmt, Pamb, Ahx, APAc, PPAc, Bal, Cmp, Pab, O2Oc, Met, and Ttds;
Xaa2 is selected from the group consisting of Aib, Ala, Amd, Ams, amd, ams, Deg, Nmg, Pam, and Pro; with the proviso that Xaa2 can be absent when L¹ is bond and NT is Hib;
Xaa3 is selected from the group consisting of Phe, Nmf, 1Ni, 2Ni, 6Clw, Cys(Bzl), Hfe, Trp, Mpa, Opa, and Ppa, wherein Phe, Nmf, and Hfe are optionally substituted by 1 or 2 substituents independently selected from the group consisting of halogen, CH₃, CN, CF₃, and OH;
Xaa5 is selected from the group consisting of Formula (Va), Formula (Vb), Hgn, lys, Nle, Tap, Aph, and Gln: 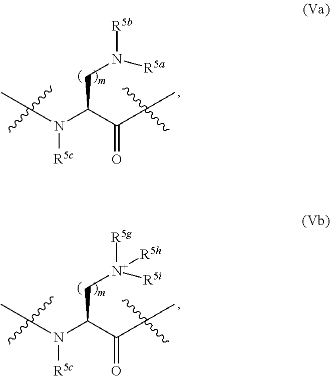
wherein R^{5a} is selected from the group consisting of H, (C₁-C₆)alkyl, Ac, C(═NR^{5d})NR^{5e}R^{5f}, and Bio;
R^{5d} is selected from the group consisting of H and CH₃;
R^{5e} and R^{5f} are independently selected from the group consisting of H and (C₁-C₆)alkyl;
R^{5b} is selected from the group consisting of H and (C₁-C₆)alkyl;
R^{5c} is selected from the group consisting of H and CH₃;
m is selected from the group consisting of 2, 3, 4, and 5; and
R^{5g}, R^{5h}, and R⁵ⁱ are independently selected from the group consisting of (C₁-C₆)alkyl;
Xaa6 is selected from the group consisting of Formula (VI), Formula (VII), Nle, and arg: 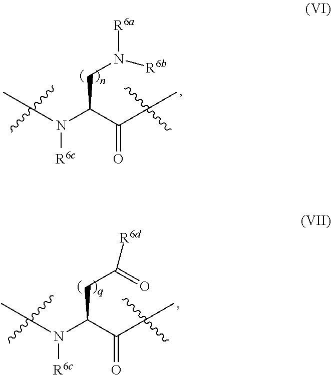
wherein R^{6a} is selected from the group consisting of H, C(═NR^{6e})NR^{6f}R^{6g}, C(═O)R^{6h}, and pyridyl;
R^{6e} is selected from the group consisting of H and CH₃;
R^{6f} is selected from the group consisting of H, (C₁-C₆)alkyl, Ac, NO₂, and C(═O)NR⁶ⁱR^{6j};
R⁶ⁱ and R^{6j} are independently selected from the group consisting of H and (C₁-C₂)alkyl;
R^{6g} is selected from the group consisting of H and (C₁-C₆)alkyl;
or alternatively, R^{6e} and R^{6f} can together form a 5 or 6 membered heterocycle;
R^{6h} is selected from the group consisting of (C₁-C₆)alkyl, NR^{6k}R^{6m}, and NR⁶ⁿC(═NR^{6p})NR^{6q}R^{6r};
R^{6k} and R^{6m} are independently selected from the group consisting of H and (C₁-C₆)alkyl;
R⁶ⁿ and R^{6p} are independently selected from the group consisting of H and CH₃;
R^{6q} and R^{6r} are independently selected from the group consisting of H and (C₁-C₆)alkyl;
R^{6b} is selected from the group consisting of H and (C₁-C₆)alkyl;
R^{6c} is selected from the group consisting of H and CH₃;
n is selected from the group consisting of 1, 2, 3, and 4;
R^{6d} is selected from the group consisting of NR^{6s}C(═NR^{6t})NR^{6u}R^{6v}, OH, and NR^{6w}R^{6x};
R^{6s} and R^{6t} are independently selected from the group consisting of H and CH₃;
R^{6u}, R^{6v}, R^{6w}, and R^{6x} are independently selected from the group consisting of H and (C₁-C₆)alkyl; and
q is selected from the group consisting of 2, 3, and 4;
Xaa7 is selected from the group consisting of Formula (VIII), Formula (IX), Formula (X), Formula (XI), Dtc, and Oic: 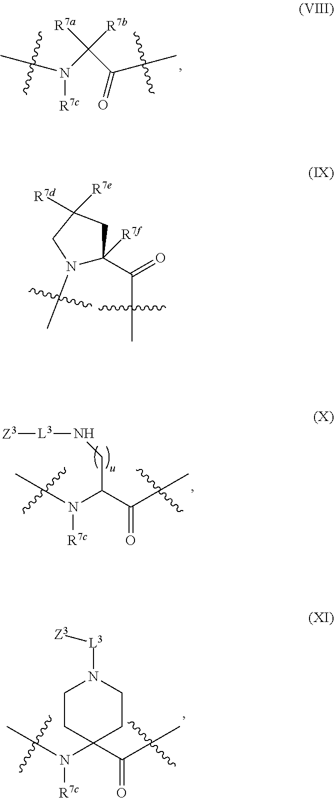
wherein R^{7a} is selected from the group consisting of H, (C₁-C₆)alkyl, and (CH₂)ₜR^{7g};
R^{7g} is selected from the group consisting of OH, CO₂H, and NR^{7h}R⁷ⁱ;
R^{7h} and R⁷ⁱ are independently selected from the group consisting of H and (C₁-C₆)alkyl;
t is selected from the group consisting of 1, 2, 3 and 4;
R^{7b} is selected from the group consisting of H and (C₁-C₆)alkyl;
or alternatively, R^{7a} and R^{7b} can together form a 5 or 6 membered carbocycle or heterocycle,
R^{7c} is selected from the group consisting of H and CH₃;
R^{7d} is selected from the group consisting of H, F, and OH;
R^{7e} is selected from the group consisting of H and F;
R^{7f} is selected from the group consisting of H and CH₃;
u is selected from the group consisting of 2, 3, and 4;
L³ is selected from the group consisting of bond and -(Xab1)ᵥ-;
v is selected from the group consisting of 1, 2, and 3;
Xab1 is each and individually selected from the group consisting of Ttds, Pamb, APAc, O2Oc, Ahx, Pab, and Cmp;
wherein, if v=1, Xab1 is covalently bound to Z³ and covalently bound to a side chain amino function of Formula (X) or of Formula (XI),
if v=2, a first of the two Xab1 is covalently bound to Z³ and covalently bound to a second of the two Xab1, and the second of the two Xab1 is covalently bound to the first of the two Xab1 and covalently bound to a side chain amino function of Formula (X) or of Formula (XI),
if v=3, a first of the three Xab1 is covalently bound to Z³ and covalently bound to a second of the three Xab1, the second of the three Xab1 is covalently bound to the first of the three Xab1 and covalently bound to a third of the three Xab1, and the third of the three Xab1 is covalently bound to the second of the three Xab1 and covalently bound to a side chain amino function of Formula (X) or of Formula (XI);
Z³ is selected from the group consisting of H and chelator;
Xaa8 is an α-amino acid residue, wherein the α-nitrogen atom of Xaa8 is unsubstituted or is optionally substituted by CH₃;
Xaa10 is Formula (XII) 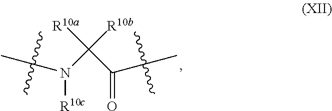
wherein
R^{10a} is selected from the group consisting of (C₁-C₆)alkyl;
R^{10b} is selected from the group consisting of H and CH₃;
R^{10c} is selected from the group consisting of H and CH₃;
L² is selected from the group consisting of
bond and -Xaa11-(Xaa12)ₛ-, such that Xaa11 is covalently bound to Xaa10;
Xaa11 is selected from the group consisting of Thr, Ala, Bal, Gab, Gln, Glu, Gly, Leu, Nmt, Phe, Pro, and Trp;
s is selected from the group consisting of 0, 1, 2, 3, 4, and 5; and
Xaa12 is each and individually selected from the group consisting of Asp, asp, Ala, Gab, Ttds, Pamb, Cmp, O2Oc, APAc, Gly, Ser, Lys(Bio), and Pab;
wherein, if s=0, Xaa11 is covalently bound to Z²,
if s=1, Xaa12 is covalently bound to Xaa11 and covalently bound to Z²,
if s=2, a first of the two Xaa12 is covalently bound to Xaa11 and covalently bound to a second of the two Xaa12, and the second of the two Xaa12 is covalently bound to the first of the two Xaa12 and covalently bound to Z²,
if s=3, a first of the three Xaa12 is covalently bound to Xaa11 and covalently bound to a second of the three Xaa12, the second of the three Xaa12 is covalently bound to the first of the three Xaa12 and covalently bound to a third of the three Xaa12, and the third of the three Xaa12 is covalently bound to the second of the three Xaa12 and covalently bound to Z²,
if s=4, a first of the four Xaa12 is covalently bound to Xaa11 and covalently bound to a second of the four Xaa12, the second of the four Xaa12 is covalently bound to the first of the four Xaa12 and covalently bound to a third of the four Xaa12, the third of the four Xaa12 is covalently bound to the second of the four Xaa12 and covalently bound to a fourth of the four Xaa12, and the fourth of the four Xaa12 is covalently bound to the third of the four Xaa12 and covalently bound to Z²,
if s=5, a first of the five Xaa12 is covalently bound to Xaa11 and covalently bound to a second of the five Xaa12, the second of the five Xaa12 is covalently bound to the first of the five Xaa12 and covalently bound to a third of the five Xaa12, the third of the five Xaa12 is covalently bound to the second of the five Xaa12 and covalently bound to a fourth of the five Xaa12, the fourth of the five Xaa12 is covalently bound to the third of the five Xaa12 and covalently bound to a fifth of the five Xaa12, and the fifth of the five Xaa12 is covalently bound to the fourth of the five Xaa12 and covalently bound to Z²;
Z² is selected from the group consisting of CT, -en-chelator, -Ape-chelator, and Formula (CT-I) 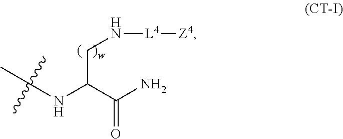
CT is selected from the group consisting of NH₂, en, en(Me)₂, en(Me), NHBu, NHnPen, AF488N3K-NH₂, OH, and Throl-OH;
w is selected from the group consisting of 2, 3, and 4;
L⁴ is selected from the group consisting of a bond, Ttds, Pamb, APAc, O2Oc, Ahx, Pab, and Cmp;
Z⁴ is chelator;
with the proviso that Xaa10 can be absent if L² is bond and CT is NHnPen;
and wherein the compound of Formula (I) may optionally comprise a therapeutically active nuclide or a diagnostically active nuclide.

3. The compound of claim 1, or a pharmaceutically acceptable salt, a pharmaceutically acceptable solvate or a hydrate thereof, wherein only 1 of Z¹, Z², Z³, and Z⁴ comprises a chelator.

4. The compound, or a pharmaceutically acceptable salt, a pharmaceutically acceptable solvate or a hydrate thereof, of claim 1, wherein
Z² is CT; and
Xaa7 is selected from the group consisting of Formula (VIII), Formula (IX), Dtc, and Oic.

5. The compound of claim 4, or a pharmaceutically acceptable salt, a pharmaceutically acceptable solvate or a hydrate thereof wherein Z¹ is chelator.

6. The compound of claim 4, or a pharmaceutically acceptable salt, a pharmaceutically acceptable solvate or a hydrate thereof, wherein L¹ is -(Xaa1)ₖ-; and wherein k is selected from the group consisting of 1 and 2.

7. The compound of claim 4, or a pharmaceutically acceptable salt, a pharmaceutically acceptable solvate or a hydrate thereof, wherein k is 2, and L¹ is Formula (XIII): 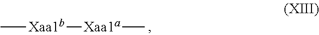
wherein Xaa1^{af66} is covalently bound to Xaa2;
Xaa1^{a} is selected from the group consisting of Thr, Ile, and Leu; and
Xaa1^{b} is selected from the group consisting of Cmp, Ttds, Pamb, Ahx, APAc, Bal, O2Oc, and Pab.

8. The compound of claim 4, or a pharmaceutically acceptable salt, a pharmaceutically acceptable solvate or a hydrate thereof, wherein k is 1, and Xaa1 is selected from the group consisting of Pamb, Bal, Cmp, Pab, Ahx, APAc, Thr, Pamp, and PPAc.

9. The compound of claim 4, or a pharmaceutically acceptable salt, a pharmaceutically acceptable solvate or a hydrate thereof, wherein L² is selected from the group consisting of bond and -Xaa11-(Xaa12)ₛ-; and s is selected from the group consisting of 0 and 1.

10. The compound of claim 9, or a pharmaceutically acceptable salt, a pharmaceutically acceptable solvate or a hydrate thereof, wherein L² is -Xaa11-(Xaa12)ₛ-;
s is selected from the group consisting of 0 and 1; and
Xaa11 is selected from the group consisting of Thr, Bal, Gln, Phe, Gab, Nmt, Gly, Leu, Trp, Glu, and Pro.

11. The compound of claim 10, or a pharmaceutically acceptable salt, a pharmaceutically acceptable solvate or a hydrate thereof, wherein s is 0.

12. The compound of claim 9, or a pharmaceutically acceptable salt, a pharmaceutically acceptable solvate or a hydrate thereof, wherein s is 1; and Xaa12 is Asp.

13. The compound of claim 9, or a pharmaceutically acceptable salt, a pharmaceutically acceptable solvate or a hydrate thereof, wherein L² is bond.

14. The compound of claim 9, or a pharmaceutically acceptable salt, a pharmaceutically acceptable solvate or a hydrate thereof, wherein CT is selected from the group consisting of NH₂, en, en(Me), en(Me)₂, NHBu, Throl-OH, and OH.

15. The compound of claim 4, or a pharmaceutically acceptable salt, a pharmaceutically acceptable solvate or a hydrate thereof, wherein L²-Z² is selected from the group consisting of Thr-NH₂, Bal-NH₂, Glu-NH₂, Pro-NH₂, Gln-NH₂, Trp-NH₂, Leu-NH₂, Gly-NH₂ and Nmt-NH₂.

16. The compound of claim 4, or a pharmaceutically acceptable salt, a pharmaceutically acceptable solvate or a hydrate thereof, wherein Xaa7 is selected from the group consisting of Formula (VIII), Formula (IX), Dtc, and Oic, wherein
R^{7a} is selected from the group consisting of H, (C₁-C₆)alkyl, and (CH₂)ₜR^{7g};
t is selected from the group consisting of 1 and 2; and
R^{7g} is selected from the group consisting of OH, CO₂H, and NH₂,
preferably Xaa7 is selected from the group consisting of Aib, Ala, Glu, Pro, Dfp, glu, Amd, 4Tfp, Pam, Deg, Nmg, Ams, ams, amd, Dtc, and Oic, or
preferably Xaa7 is selected from Formula (VIII), and wherein
R^{7a} is selected from the group consisting of H, (C₁-C₂)alkyl, CH₂OH, CH₂CO₂H, and CH₂CH₂CO₂H;
R^{7b} is selected from the group consisting of H and (C₁-C₂)alkyl; and
R^{7c} is H; more preferably wherein Xaa7 is selected from the group consisting of Aib and Ala.

17. A compound, or a pharmaceutically acceptable salt, a pharmaceutically acceptable solvate or a hydrate thereof, of claim 1, wherein
Z¹ is NT, and
Xaa7 is selected from the group consisting of Formula (VIII), Formula (IX), Dtc, and Oic.

18. A compound of claim 17, or a pharmaceutically acceptable salt, a pharmaceutically acceptable solvate or a hydrate thereof, wherein Z² is selected from the group consisting of en-chelator, -Ape-chelator, and Formula (CT-I), wherein if Z² is Formula (CT-I), then Z⁴ is chelator.

19. A compound of claim 17, or a pharmaceutically acceptable salt, a pharmaceutically acceptable solvate or a hydrate thereof, wherein
L¹ is selected from the group consisting of bond and (Xaa1)ₖ; and
k is selected from the group consisting of 1 and 2.

20. The compound of claim 19, or a pharmaceutically acceptable salt, a pharmaceutically acceptable solvate or a hydrate thereof, wherein k is 2 and L¹ is Formula (XIII) 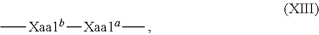
wherein Xaa1a is covalently bound to Xaa2 of Formula (I);
Xaa1^{a} is Thr; and
Xaa1^{b} is Met or Cmp.

21. The compound of claim 20, or a pharmaceutically acceptable salt, a pharmaceutically acceptable solvate or a hydrate thereof, wherein Xaa1^{b} is Met.

22. The compound of a claim 20, or a pharmaceutically acceptable salt, a pharmaceutically acceptable solvate or a hydrate thereof, wherein NT is selected from the group consisting of H, Ac, and nBuCAyl.

23. The compound of claim 19, or a pharmaceutically acceptable salt, a pharmaceutically acceptable solvate or a hydrate thereof, wherein k is 1, and Xaa1 is selected from the group consisting of Thr, Ala, Pamp, and Ser.

24. The compound of claim 23, or a pharmaceutically acceptable salt, a pharmaceutically acceptable solvate or a hydrate thereof, wherein Xaa1 is selected from the group consisting of Thr and Pamp; and NT is selected from the group consisting of Ac, nBuCAyl, and Hex.

25. The compound of claim 19, or a pharmaceutically acceptable salt, a pharmaceutically acceptable solvate or a hydrate thereof, wherein L¹ is bond, preferably NT is selected from the group consisting of Ac, HPA, HYDAc, Iva, SaPr, and HO-Succinyl.

26. The compound of claim 17, or a pharmaceutically acceptable salt, a pharmaceutically acceptable solvate or a hydrate thereof, wherein
L² is -Xaa11-(Xaa12)ₛ-; and
s is selected from the group consisting of 0, 1, and 2.

27. The compound of claim 26, or a pharmaceutically acceptable salt, a pharmaceutically acceptable solvate or a hydrate thereof, wherein Z² is Formula (CT-I).

28. The compound of claim 26, or a pharmaceutically acceptable salt, a pharmaceutically acceptable solvate or a hydrate thereof, wherein Xaa11 is Thr.

29. The compound of claim 26, or a pharmaceutically acceptable salt, a pharmaceutically acceptable solvate or a hydrate thereof, wherein L² is
a)
-Xaa11-(Xaa12)ₛ; s is 1; and
Xaa12 is selected from the group consisting of Asp, Cmp, Ttds, Pamb, O2Oc, APAc, and Pab,
or
b)
-Xaa11-(Xaa12)ₛ; and s is 2; and L² has the structure
-Xaa11-Xaa12^{a}-Xaa12^{b}-; wherein
Xaa12^{a} is selected from the group consisting of Asp, Cmp, Ttds, Pamb, O2Oc, APAc, and Pab; and
Xaa12^{b} is Ttds.

30. The compound of claim 17, or a pharmaceutically acceptable salt, a pharmaceutically acceptable solvate or a hydrate thereof, wherein Z² is selected from the group consisting of -en-chelator and -Ape-chelator.

31. The compound of claim 30, or a pharmaceutically acceptable salt, a pharmaceutically acceptable solvate or a hydrate thereof, wherein L² is a bond or Xaa11, wherein if L² is Xaa11, Xaa11 is preferably Thr.

32. The compound of claim 17, or a pharmaceutically acceptable salt, a pharmaceutically acceptable solvate or a hydrate thereof, wherein Xaa7 is selected from Formula (VIII), Formula (IX), Dtc, and Oic, wherein
R^{7a} is selected from the group consisting of H, (C₁-C₆) alkyl, and (CH₂)ₜR^{7g};
R^{7g} is selected from the group consisting of OH and CO₂H;
t is selected from the group consisting of 1 and 2; and
R^{7b} is selected from the group consisting of H and (C₁-C₆) alkyl; preferably Xaa7 is
a)
selected from the group consisting of Aib, Ala, Glu, Pro, Dfp, glu, Amd, 4Tfp, Pam, Deg, Nmg, Ams, ams, amd, Dtc, and Oic;
or
b)
of Formula (VIII), and wherein
R^{7a} is selected from the group consisting of H, (C₁-C₂)alkyl, CH₂OH, CH₂CO₂H, and CH₂CH₂CO₂H;
R^{7b} is selected from the group consisting of H and (C₁-C₂)alkyl; and
R^{7c} is H.

33. The compound of claim 32, or a pharmaceutically acceptable salt, a pharmaceutically acceptable solvate or a hydrate thereof, wherein Xaa7 is selected from the group consisting of Ala and Aib.

34. A compound, or a pharmaceutically acceptable salt, a pharmaceutically acceptable solvate or a hydrate thereof, of claim 1, wherein Xaa7 is selected from the group consisting of Formula (X) and Formula (XI);
Z³ is chelator;
Z¹ is NT; and
Z² is CT.

35. The compound of claim 34, or a pharmaceutically acceptable salt, a pharmaceutically acceptable solvate or a hydrate thereof, wherein
L¹ is selected from the group consisting of bond and -(Xaa1)ₖ-; and k is 1, wherein if L¹ is Xaa1, Xaa1 is preferably Thr.

36. The compound of claim 34, or a pharmaceutically acceptable salt, a pharmaceutically acceptable solvate or a hydrate thereof, wherein NT is selected from the group consisting of Ac, SaPr, Iva, and HPA.

37. The compound of claim 34, or a pharmaceutically acceptable salt, a pharmaceutically acceptable solvate or a hydrate thereof, wherein L² is selected from the group consisting of bond and -Xaa11-(Xaa12)ₛ-, wherein s is selected from the group consisting of 0 and 1; preferably Xaa11 is selected from the group consisting of Thr, Gln, Phe, Gab, Nmt, Bal, Gly, Leu, Trp, Glu, and Pro.

38. The compound of claim 37, or a pharmaceutically acceptable salt, a pharmaceutically acceptable solvate or a hydrate thereof, wherein CT is NH₂.

39. The compound of claim 34, or a pharmaceutically acceptable salt, a pharmaceutically acceptable solvate or a hydrate thereof, wherein Xaa7 is selected from the group consisting of Lys and Apc, wherein to the ε-nitrogen atom of Lys or the γ-nitrogen atom of Apc a chelator is attached, wherein an optional linker is interspersed between Apc or Lys and the chelator.

40. The compound of claim 34, or a pharmaceutically acceptable salt, a pharmaceutically acceptable solvate or a hydrate thereof, wherein Xaa7 is selected from the group consisting of Apc(DOTA), Lys(DOTAGA-O2Oc), Lys(DOTA-O2Oc), Lys(DOTA-Pab), Lys(DOTA-Ahx), Lys(DOTA-APAc), Lys(DOTA-Pamb), Lys(DOTA-Cmp), Lys(DOTA-Ttds), Lys(DOTA).

41. The compound of claim 34, or a pharmaceutically acceptable salt, a pharmaceutically acceptable solvate or a hydrate thereof, wherein Xaa7 is selected from Formula (X), wherein
u is 4;
L³ is -(Xab1)ᵥ-;
v is 1; and
Xab1 is selected from the group consisting of Ttds, Pamb, APAc, O2Oc, Ahx, and Pab.

42. The compound of claim 1, or a pharmaceutically acceptable salt, a pharmaceutically acceptable solvate or a hydrate thereof, wherein chelator is selected from the group consisting of DOTA, DOTAGA, LSC, NOPO, PCTA, DOTAM, Macropa, Crown, NOTA, and NODAGA.

43. The compound of claim 1, or a pharmaceutically acceptable salt, a pharmaceutically acceptable solvate or a hydrate thereof, wherein Xaa2 is selected from the group consisting of Aib, Ala, Ams, ams, Deg, Pam, and Pro.

44. The compound of claim 1, or a pharmaceutically acceptable salt, a pharmaceutically acceptable solvate or a hydrate thereof, wherein Xaa3 is selected from the group consisting of Phe, 1Ni, 2Ni, 6Clw, Cys(Bzl), Hfe, and Trp, wherein Phe, Nmf, and Hfe are optionally substituted by 1 or 2 substituents independently selected from the group consisting of Cl, CH₃, F, CN, CF₃, and OH; preferably Xaa3 is selected from the group consisting of Phe and Pcf.

45. The compound of claim 1, or a pharmaceutically acceptable salt, a pharmaceutically acceptable solvate or a hydrate thereof, wherein Xaa5 is selected from the group consisting of Formula (Va), Formula (Vb), Hgn, and Nle, and R^{5g}, R^{5h}, and R⁵ⁱ are CH₃;
preferably Xaa5 is selected from Formula (Va), and
R^{5a} is selected from the group consisting of H, CH₃, Ac, and C(═NR^{5d})NR^{5e}R^{5f};
R^{5e} and R^{5f} are independently selected from the group consisting of H and CH₃;
R^{5b} is H;
R^{5c} is H; and
m is selected from the group consisting of 3 and 4;
more preferably Xaa5 is selected from the group consisting of Lys, Lys(Me), Kip and KMe3.

46. The compound of claim 1, or a pharmaceutically acceptable salt, a pharmaceutically acceptable solvate or a hydrate thereof, wherein Xaa6 is selected from the group consisting of Formula (VI), Formula (VII), and Nle,
wherein R^{6a} is selected from the group consisting of H, C(═NR^{6e})NR^{6f}R^{6g}, and C(═O)R^{6h};
R^{6e} is H;
R^{6f} is selected from the group consisting of H, CH₃, Ac, NO₂, and C(═O)NR⁶ⁱR^{6j};
R^{6g} is selected from the group consisting of H and CH₃;
R^{6h} is selected from the group consisting of CH₃, NH₂, and NHC(═NH)NH₂;
R^{6b} is H;
R^{6c} is H; and
R^{6d} is selected from the group consisting of NHC(═NH)NH₂ and NH₂;
preferably Xaa6 is
a) selected from the group consisting of Arg, Arg(Me), Cit, Egd, RMe2a, RMe3, Nle, Gln, Lys(Ac), Hgn, Arg(EtCAyl), Urr, Arg(Ac), Gln(Gu), Orn, Har, RMe2, and Eew;
or
b) is of Formula (VI), and wherein
n is 3;
R^{6a} is C(═NH)NHR^{6f}; and
R^{6f} is selected from the group consisting of H, Ac, NO₂, and CH₃.

47. The compound of claim 46, or a pharmaceutically acceptable salt, a pharmaceutically acceptable solvate or a hydrate thereof, wherein Xaa6 is selected from the group consisting of Arg, Arg(Me), Cit, Egd, RMe2a and RMe3.

48. The compound of claim 1, or a pharmaceutically acceptable salt, a pharmaceutically acceptable solvate or a hydrate thereof, wherein Xaa8 is selected from the group consisting of Formula (XIV), Gly, Val, Met, Ile and Thr: 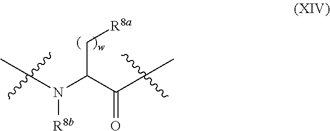
wherein R^{8a}, is selected from the group consisting of H, OH, NH₂, COOH, C(═O)NH₂, NHC(═NH)NH₂, (C₁-C₈)alkyl, aryl, and heteroaryl;
w is selected from the group consisting of 1, 2, and 3; and
R^{8b} is selected from the group consisting of H and CH₃;
preferably R^{8a} is selected from the group consisting of OH, COOH, C(═O)NH₂, phenyl, CH₂NHC(═NH)NH₂, indole, and CH(CH₃)₂; and
w is selected from the group consisting of 1 and 2.

49. The compound of claim 48, or a pharmaceutically acceptable salt, a pharmaceutically acceptable solvate or a hydrate thereof, wherein Xaa8 is selected from the group consisting of Asn, Trp, Phe, Arg, Ser, Gly, Leu, Asp, Nmn, Glu, and asn.

50. The compound of claim 1, or a pharmaceutically acceptable salt, a pharmaceutically acceptable solvate or a hydrate thereof, wherein Xaa10 is Formula (XII): 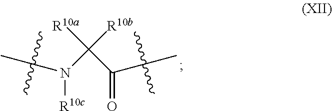
wherein
R^{10b} is selected from the group consisting of H and CH₃;
R^{10c} is H;
preferably Xaa10 is selected from the group consisting of Tle, Leu, Val, Npg, and Ile.

51. The compound of claim 1, or a pharmaceutically acceptable salt, a pharmaceutically acceptable solvate or a hydrate thereof, wherein the compound is a compound of Formula (Ia).

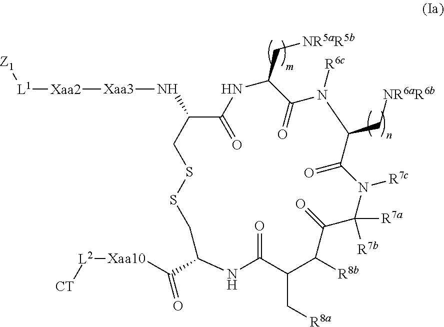

52. The compound of claim 51, or a pharmaceutically acceptable salt, a pharmaceutically acceptable solvate or a hydrate thereof, wherein Xaa3 is selected from the group consisting of Phe, 1Ni, 2Ni, 6Clw, Cys(Bzl), Hfe, and Trp, wherein Phe, Nmf, and Hfe are optionally substituted by 1 or 2 substituents independently selected from the group consisting of Cl, CH₃, F, CN, CF₃, and OH; preferably Xaa3 is selected from the group consisting of Phe and Pcf.

53. The compound of claim 51, or a pharmaceutically acceptable salt, a pharmaceutically acceptable solvate or a hydrate thereof, wherein Xaa10 is Formula (XII), wherein 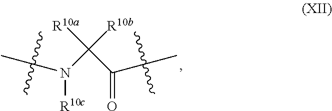
R^{10b} is selected from the group consisting of H and CH₃;
R^{10c} is H;
preferably Xaa10 is selected from the group consisting of a compound of Formula (XIV) 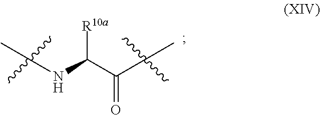
more preferably R^{10a} is selected from the group consisting of C(CH₃)₃, CH₂CH(CH₃)₂, CH(CH₃)₂, CH(CH₃)C₂H₅ and CH₂C(CH₃)₃.

54. The compound of claim 51, or a pharmaceutically acceptable salt, a pharmaceutically acceptable solvate or a hydrate thereof, wherein the compound is a compound of Formula (Ib) 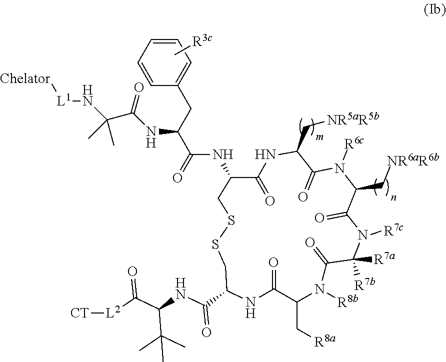
wherein R^{3c} is selected from the group consisting of H, Cl, CH₃, F, CN, CF₃, and OH; and
R^{3c} is at the meta or para position of the phenyl ring of Formula (Ib).

55. The compound of claim 1, or a pharmaceutically acceptable salt, a pharmaceutically acceptable solvate or a hydrate thereof, wherein
Xaa2 is Aib or Ala;
Xaa3 is Phe or Pcf;
Xaa5 is Lys(Me), Lys, Kip or KMe3;
Xaa6 is Arg(Me), Arg, Egd, Cit, RMe2a or RMe3;
Xaa7 is Aib or Ala;
Xaa8 is Asn; and
Xaa10 is Tle or Leu.

56. The compound of claim 17, or pharmaceutically acceptable salt, solvate or hydrate thereof, wherein
Xaa2 is Aib or Ala;
Xaa3 is Phe or Pcf;
Xaa5 is Lys or Lys(Me);
Xaa6 is Arg or Arg(Me);
Xaa7 is Aib or Ala;
Xaa8 is Ans; and
Xaa10 is Tle or Leu.

57. The compound of claim 1, or pharmaceutically acceptable salt, solvate or hydrate thereof, wherein the compound is selected from the following
DOTA-Cmp-Thr-Aib-Pcf-[Cys-Lys(Me)-Arg(Me)-Aib-Asn-Cys+-Tle-Thr-NH₂ (PSM-0194)_{;}
DOTA-Cmp-Thr-Aib-Pcf-[Cys-Lys-Arg-Aib-Asn-Cys+-Tle-NH₂ (PSM-0433);
DOTA-Cmp-Thr-Aib-Phe-[Cys-Lys-Arg-Aib-Asn-Cys+-Tle-Thr-NH₂ (PSM-0492);
Ac-Thr-Aib-Phe-[Cys-Lys-Arg-Aib-Asn-Cys+-Tle-Thr-NH₂ (PSM-0178);
Ac-Thr-Aib-Pcf-[Cys-Lys-Cit-Aib-Asn-Cys+-Tle-Thr-NH₂ (PSM-0179);
Ac-Thr-Aib-Phe-[Cys-Lys-Gln-Ala-Asn-Cys+-Tle-Thr-Asp-NH₂ (PSM-0180);
Ac-Thr-Aib-Phe-[Cys-Lys-Arg-Hyp-Asn-Cys+-Tle-Thr-Asp-NH₂ (PSM-0181);
Ac-Thr-Aib-Phe-[Cys-Lys-Arg-Aib-Asn-Cys+-Tle-Thr-Asp-Ttds-Ttds-AF488N3K-NH₂ (PSM-0183)_{;}
DOTA-Thr-Aib-Phe-[Cys-Lys-Arg-Aib-Asn-Cys+-Tle-NH₂ (PSM-0184);
Ac-Pamp-Aib-Pcf-[Cys-Lys-Arg-Aib-Asn-Cys+-Tle-en-DOTA (PSM-0186);
Ac-Thr-Deg-Phe-[Cys-Lys-Arg-Aib-Asn-Cys+-Tle-Thr-NH₂ (PSM-0187);
SaPr-Aib-Pcf-[Cys-Lys-Arg-Lys(DOTA-APAc)-Asn-Cys+-Tle-Thr-NH₂ (PSM-0188);
Ac-Thr-Aib-Pcf-[Cys-Lys-Gln(Gu)-Aib-Asn-Cys+-Tle-Thr-NH₂ (PSM-0189);
Ac-Thr-Aib-Pcf-[Cys-Lys-Arg-Aib-Asn-Cys+-Tle-Thr-lys(DOTA-Cmp)-NH₂ (PSM-0190);
Ac-Thr-Aib-Phe-[Cys-Lys-Arg-Aib-Asn-Cys+-Tle-Thr-APAc-lys(DOTA)-NH₂ (PSM-0191);
DOTA-Ttds-Thr-Aib-Phe-[Cys-Lys-Arg-Ala-Asn-Cys+-Tle-Thr-Asp-NH₂ (PSM-0193);
Ac-Thr-Ala-Phe-[Cys-Lys-Arg-Glu-Asn-Cys+-Nle-Thr-Asp-NH₂ (PSM-0197);
DOTA-Cmp-Tle-Aib-Phe-[Cys-Lys-Arg-Aib-Asn-Cys+-Tle-Thr-NH₂ (PSM-0198);
DOTA-Thr-Aib-Phe-[Cys-Lys-Arg-Aib-Asn-Cys+-Tle-en (PSM-0199);
DOTA-Cmp-Thr-Aib-Pcf-[Cys-Lys(Me)-Arg-Aib-Asn-Cys+-Tle-NH₂ (PSM-0200);
DOTA-Pamb-Thr-Aib-Phe-[Cys-Lys-Arg-Aib-Asn-Cys+-Tle-Thr-NH₂ (PSM-0202);
DOTA-Cmp-Thr-Aib-Pcf-[Cys-KMe2-Arg(Me)-Aib-Asn-Cys+-Tle-Thr-NH₂ (PSM-0203);
nBuCAyl-Thr-Aib-Pcf-[Cys-Lys-Cit-Aib-Asn-Cys+-Tle-Thr-Cmp-lys(DOTA)-NH₂ (PSM-0204);
Ac-Thr-Aib-Pcf-[Cys-Nle-Arg-Aib-Asn-Cys+-Tle-Thr-NH₂ (PSM-0205);
nBuCAyl-Thr-Aib-Mcf-[Cys-Lys-Cit-Aib-Asn-Cys]-Tle-Thr-Cmp-lys(DOTA)-NH₂ (PSM-0207);
Ac-Thr-Aib-Phe-[Cys-Lys-Arg-Aib-Asn-Cys]-NHnPen (PSM-0208);
DOTA-Bal-Thr-Aib-Phe-[Cys-Lys-Arg-Aib-Asn-Cys]-Tle-Thr-NH₂ (PSM-0209);
Ac-Thr-Aib-Phe-[Cys-Gln-Arg-Aib-Asn-Cys]-Tle-Thr-Asp-NH₂ (PSM-0210);
Ac-Thr-Aib-Phe-[Cys-Lys-Arg-Lys(DOTA-Pab)-Asn-Cys]-Tle-Thr-NH₂ (PSM-0211);
Ac-Thr-Aib-Phe-[Cys-Lys-Arg-Aib-Asn-Cys]-Tle-Thr-Asp-Ttds-lys(DOTA)-NH₂ (PSM-0212);
SaPr-Aib-Pcf-[Cys-Lys-Arg-Aib-Asn-Cys]-Tle-Thr-Cmp-lys(DOTA)-NH₂ (PSM-0215);
DOTA-Thr-Aib-Pcf-[Cys-Lys-Arg-Aib-Asn-Cys]-Tle-Bal-NH₂ (PSM-0216);
Ac-Thr-Aib-Phe-[Cys-Lys-Arg-Dfp-Asn-Cys]-Tle-Thr-Asp-NH₂ (PSM-0217);
DOTAGA-Cmp-Thr-Aib-Phe-[Cys-Lys-Arg-Aib-Asn-Cys]-Tle-Thr-NH₂ (PSM-0218);
DOTA-Cmp-Leu-Aib-Phe-[Cys-Lys-Arg-Aib-Asn-Cys]-Tle-Thr-NH₂ (PSM-0220);
DOTA-Pamb-Aib-Phe-[Cys-Lys-Arg-Aib-Asn-Cys]-Tle-en (PSM-0221);
DOTA-Thr-Aib-Pcf-[Cys-Lys-Arg-Aib-Asn-Cys]-Tle-en (PSM-0222);
DOTA-Cmp-Aib-Pcf-[Cys-Lys-Arg(Ac)-Aib-Asn-Cys]-Tle-Thr-NH₂ (PSM-0223);
SaPr-Aib-Phe-[Cys-Lys-Arg-Aib-Asn-Cys]-Tle-Thr-NH₂ (PSM-0224);
DOTA-Cmp-Thr-Aib-Pcf-[Cys-Har-Arg-Aib-Asn-Cys]-Tle-Thr-NH₂ (PSM-0225);
H-Met-Thr-Ala-Phe-[Cys-Lys-Arg-Ala-Asn-Cys]-Leu-Thr-Asp-Gly-Ser-Ttds-Ttds-
Ttds-Lys(Bio)-NH₂ (PSM-0226);
DOTA-Cmp-Thr-Aib-Pcf-[Cys-Lys-Arg-amd-Asn-Cys]-Tle-Thr-NH₂ (PSM-0227);
Ac-Aib-Phe-[Cys-Lys-Arg-Aib-Asn-Cys]-Tle-Thr-NH₂ (PSM-0228);
Ac-Thr-Aib-Pcf-[Cys-Lys-Arg-Aib-Met-Cys]-Tle-Thr-NH₂ (PSM-0229);
Ac-Thr-Aib-Phe-[Cys-Lys-Arg-Aib-Asn-Cys]-Tle-NH₂ (PSM-0230);
Ac-Thr-Aib-Phe-[Cys-Lys-Arg-Ala-Asn-Cys]-Tle-Thr-Ttds-lys(DOTA)-NH₂ (PSM-0231);
Ac-Thr-Aib-Phe-[Cys-Lys-Arg-Nmg-Asn-Cys]-Tle-Thr-NH₂ (PSM-0232);
Ac-Thr-Aib-Phe-[Cys-Lys-Arg-Pam-Asn-Cys]-Tle-Thr-NH₂ (PSM-0233);
DOTA-Ttds-Thr-Aib-Phe-[Cys-Lys-Cit-Aib-Asn-Cys]-Tle-Thr-NH₂ (PSM-0234);
Ac-Thr-Aib-Phe-[Cys-Lys-Nmr-Aib-Asn-Cys]-Tle-Thr-NH₂ (PSM-0235);
Ac-Thr-Aib-Pcf-[Cys-Nle-Gln(Gu)-Aib-Asn-Cys]-Tle-Thr-NH₂ (PSM-0236);
DOTA-Ttds-Thr-Aib-Phe-[Cys-Lys-Arg-Ala-Asn-Cys]-Tle-Thr-NH₂ (PSM-0237);
Ac-Thr-Aib-Pcf-[Cys-Lys-Cit-Aib-Trp-Cys]-Tle-Thr-NH₂ (PSM-0238);
Hex-Thr-Aib-Pcf-[Cys-Lys-Cit-Aib-Asn-Cys]-Tle-Thr-lys(DOTA-Cmp)-NH₂ (PSM-0239);
Ac-Thr-Aib-Pcf-[Cys-Lys-Cit-Aib-Arg-Cys]-Tle-Thr-NH₂ (PSM-0240);
DOTA-Cmp-Thr-Aib-Phe-[Cys-Lys-Arg-Aib-Asn-Cys]-Tle-en (PSM-0241);
Ac-Thr-Aib-Mcf-[Cys-Lys-Arg-Lys(DOTA-020c)-Asn-Cys]-Tle-Thr-NH₂ (PSM-0243);
DOTA-Cmp-Thr-Aib-Pcf-[Cys-Kip-Arg(Me)-Aib-Asn-Cys]-Tle-Thr-NH₂ (PSM-0244);
DOTA-Cmp-Thr-Aib-Pcf-[Cys-Lys-Orn-Aib-Asn-Cys]-Tle-Thr-NH₂ (PSM-0245);
DOTA-Cmp-Thr-Aib-Pcf-[Cys-Lys(Me)-Arg(Me)-ala-Asn-Cys]-Tle-Thr-NH₂ (PSM-0246);
DOTA-Cmp-Thr-Aib-Pcf-[Cys-Lys(Me)-RMe2-Aib-Asn-Cys]-Tle-Thr-NH₂ (PSM-0247);
DOTA-Cmp-Aib-Pcf-[Cys-Lys-Arg-Aib-Asn-Cys]-Tle-Thr-NH₂ (PSM-0248);
Ac-Thr-Aib-1Ni-[Cys-Lys-Arg-Aib-Asn-Cys]-Tle-Thr-Asp-NH₂ (PSM-0249);
DOTA-Cmp-Thr-Aib-Pcf-[Cys-Lys-Arg-Aib-Asn-Cys]-Tle-Thr-Ttds-Ttds-Lys(Bio)-NH₂ (PSM-0250);
DOTA-Ttds-Thr-Ala-Phe-[Cys-Lys-Arg-Ala-Asn-Cys]-Leu-Thr-Asp-NH₂ (PSM-0251);
Ac-Thr-Aib-Phe-[Cys-Lys-Cit-Aib-Asn-Cys]-Tle-Thr-Cmp-lys(DOTA)-NH₂ (PSM-0252);
Ac-Thr-Aib-Phe-[Cys-Lys-Glu-Aib-Asn-Cys]-Tle-Thr-Asp-NH₂ (PSM-0253);
DOTA-Cmp-Thr-Aib-Pcf-[Cys-Lys(Me)-Eew-Aib-Asn-Cys]-Tle-Thr-NH₂ (PSM-0254);
DOTA-Cmp-Thr-Aib-Mpa-[Cys-Lys-Cit-Aib-Asn-Cys]-Tle-Thr-NH₂ (PSM-0255);
DOTA-Cmp-Thr-ams-Pcf-[Cys-Lys-Arg-Aib-Asn-Cys]-Tle-Thr-NH₂ (PSM-0256);
DOTA-Cmp-Thr-Aib-Opa-[Cys-Lys(Me)-Arg(Me)-Aib-Asn-Cys]-Tle-Thr-NH₂ (PSM-0257);
nBuCAyl-Thr-Aib-Phe-[Cys-Lys-Opy-Aib-Asn-Cys]-Tle-Thr-Cmp-lys(DOTA)-NH₂ (PSM-0258);
DOTA-Ttds-Thr-Aib-Phe-[Cys-Lys-Arg-Aib-Asn-Cys]-Tle-Thr-NH₂ (PSM-0259);
DOTA-Cmp-Thr-Aib-Pcf-[Cys-Lys-Arg-glu-Asn-Cys]-Tle-Thr-NH₂ (PSM-0260);
DOTA-APAc-Aib-Phe-[Cys-Lys-Arg-Aib-Asn-Cys]-Tle-Thr-NH₂ (PSM-0261);
Ac-Thr-Aib-Phe-[Cys-Lys-Arg-Aib-Asn-Cys]-Tle-Glu-NH₂ (PSM-0262);
DOTA-APAc-Aib-Pcf-[Cys-Lys-Arg-Aib-Asn-Cys]-Tle-Thr-NH₂ (PSM-0263);
Ac-Thr-Aib-Mtf-[Cys-Lys-Arg-Aib-Asn-Cys]-Tle-Thr-NH₂ (PSM-0264);
Ac-Thr-Ala-Phe-[Cys-Lys-Arg-Ala-Asn-Cys]-Tle-Thr-Asp-Ttds-lys(DOTA)-NH₂ (PSM-0266);
HPA-Aib-Pcf-[Cys-Lys-Arg-Aib-Asn-Cys]-Tle-Thr-lys(DOTA)-NH₂ (PSM-0267);
Ac-Thr-Aib-Pcf-[Cys-Lys-Arg-Aib-Asn-Cys]-Tle-en-DOTA (PSM-0269);
DOTA-Pamb-Aib-Phe-[Cys-Lys-Arg-Aib-Asn-Cys]-Tle-Thr-NH₂ (PSM-0270);
AF488Ahx-Ttds-Ttds-Thr-Aib-Phe-[Cys-Lys-Arg-Aib-Asn-Cys]-Tle-Thr-Asp-NH₂ (PSM-0272);
DOTA-Pamb-Aib-Pcf-[Cys-Lys-Arg-Aib-Asn-Cys]-Tle-en (PSM-0273);
DOTA-Cmp-Thr-Aib-Eaa-[Cys-Lys-Cit-Aib-Asn-Cys]-Tle-Thr-NH₂ (PSM-0274);
Ac-Thr-Aib-Mcf-[Cys-Lys-Cit-Aib-Asn-Cys]-Tle-Thr-Cmp-lys(DOTA)-NH₂ (PSM-0275);
Ac-Thr-Aib-Pnf-[Cys-Lys-Arg-Aib-Asn-Cys]-Tle-Thr-NH₂ (PSM-0278);
Ac-Thr-Aib-Phe-[Cys-Lys-Arg-Aib-Ser-Cys]-Tle-Thr-NH₂ (PSM-0279);
Ac-Thr-Aib-Phe-[Cys-Lys-Cit-Aib-Asn-Cys]-Tle-Thr-Asp-NH₂ (PSM-0280);
Ac-Aib-Pcf-[Cys-Lys-Arg-Lys(DOTA-APAc)-Asn-Cys]-Tle-Thr-NH₂ (PSM-0282);
DOTA-Pamb-Aib-Pcf-[Cys-Lys-Arg-Aib-Asn-Cys]-Tle-Thr-NH₂ (PSM-0283);
Ac-Thr-Aib-Mcf-[Cys-Lys-Cit-Lys(DOTA-020c)-Asn-Cys]-Tle-Thr-NH₂ (PSM-0284);
Ac-Thr-Aib-Phe-[Cys-Lys-Arg-Aib-Asn-Cys]-Tle-Thr-Ttds-lys(DOTA)-NH₂ (PSM-0285);
Ac-Thr-Ala-Phe-[Cys-Lys-Arg-Ala-Asn-Cys]-Nle-Thr-Asp-NH₂ (PSM-0287);
Ac-Thr-Aib-Phe-[Cys-Lys-Arg-Aib-Asn-Cys]-Tle-Thr-lys(DOTA)-NH₂ (PSM-0288);
Hex-Thr-Aib-Phe-[Cys-Lys-Gln-Aib-Asn-Cys]-Tle-Thr-Ttds-lys(DOTA)-NH₂ (PSM-0289);
Iva-Aib-Phe-[Cys-Lys-Arg-Aib-Asn-Cys]-Tle-Thr-lys(DOTA)-NH₂ (PSM-0292);
Ac-Thr-Aib-Pcf-[Cys-Lys-Cit-Aib-Ser-Cys]-Tle-Thr-NH₂ (PSM-0293);
Ac-Thr-Aib-Pff-[Cys-Lys-Arg-Aib-Asn-Cys]-Tle-Thr-NH₂ (PSM-0294);
Ac-Thr-Aib-Phe-[Cys-Lys-Arg-Ala-Asn-Cys]-Tle-Thr-Asp-Ttds-lys(DOTA)-NH₂ (PSM-0295);
DOTA-Cmp-Thr-Aib-Mpa-[Cys-Lys(Me)-Arg(Me)-Aib-Asn-Cys]-Tle-Thr-NH₂ (PSM-0296);
Ac-Thr-Aib-Phe-[Cys-Lys-Arg-Aib-Asn-Cys]-Tle-Gab-NH₂ (PSM-0297);
Ac-Aib-Pcf-[Cys-Lys-Arg(Ac)-Aib-Asn-Cys]-Tle-Thr-Cmp-lys(DOTA)-NH₂ (PSM-0298);
Ac-Ala-Phe-[Cys-Lys-Arg-Ala-Asn-Cys]-Leu-Thr-Asp-Ttds-lys(DOTA)-NH₂ (PSM-0299);
Ac-Thr-Aib-Phe-[Cys-Lys-Arg-Aib-Asn-Cys]-Tle-OH (PSM-0300);
Ac-Thr-Aib-Mcf-[Cys-Lys-Arg-Lys(DOTAGA-020c)-Asn-Cys]-Tle-Thr-NH₂ (PSM-0301);
DOTA-Cmp-Thr-Aib-Pcf-[Cys-Lys-Arg-Amd-Asn-Cys]-Tle-Thr-NH₂ (PSM-0302);
Ac-Thr-Aib-Phe-[Cys-Lys-Gln-Aib-Asn-Cys]-Tle-Thr-Ttds-lys(DOTA)-NH₂ (PSM-0303);
DOTA-Cmp-Thr-Aib-6Clw-[Cys-Lys-Cit-Aib-Asn-Cys]-Tle-Thr-NH₂ (PSM-0304);
DOTA-Ttds-Thr-Ala-Phe-[Cys-Lys-Arg-Ala-Asn-Cys]-Leu-Thr-NH₂ (PSM-0305);
DOTA-Cmp-Thr-Aib-Pcf-[Cys-Lys-Arg-Aib-Asn-Cys]-Tle-en(Me)2 (PSM-0306);
Ac-Thr-Aib-Phe-[Cys-Lys-Cit-Ala-Asn-Cys]-Tle-Thr-Asp-NH₂ (PSM-0307);
DOTA-Cmp-Thr-Aib-5Clw-[Cys-Lys(Me)-Arg(Me)-Aib-Asn-Cys]-Tle-Thr-NH₂ (PSM-0308);
DOTA-Ahx-Aib-Pcf-[Cys-Lys-Arg-Aib-Asn-Cys]-Tle-Thr-NH₂ (PSM-0310);
Ac-Thr-Ala-Phe-[Cys-Lys-Arg-Ala-Asn-Cys]-Nle-Thr-Gab-OH (PSM-0313);
DOTA-Cmp-Thr-Aib-Pcf-[Cys-Lys-Arg-ams-Asn-Cys]-Tle-Thr-NH₂ (PSM-0314);
Ac-Thr-Aib-Phe-[Cys-Lys-arg-Ala-Asn-Cys]-Tle-Thr-Asp-NH₂ (PSM-0315);
DOTA-Pab-Aib-Phe-[Cys-Lys-Arg-Aib-Asn-Cys]-Tle-Thr-NH₂ (PSM-0316);
DOTA-Cmp-Thr-Aib-Pcf-[Cys-Lys-Har-Aib-Asn-Cys]-Tle-Thr-NH₂ (PSM-0317);
Ac-Thr-Aib-Pcf-[Cys-Lys-Arg-Aib-Asn-Cys]-Tle-Thr-Asp-NH₂ (PSM-0318);
Ac-Thr-Ala-Nmf-[Cys-Lys-Arg-Aib-Asn-Cys]-Tle-Thr-NH₂ (PSM-0319);
Ac-Aib-Pcf-[Cys-Lys-Arg(Me)-Aib-Asn-Cys]-Tle-en-DOTA (PSM-0320);
Ac-Thr-Aib-Phe-[Cys-Lys-Arg-Apc(DOTA)-Asn-Cys]-Tle-Thr-NH₂ (PSM-0321);
Ac-Thr-Aib-Pcf-[Cys-Lys-Cit-Aib-Asn-Cys]-Tle-Thr-lys(DOTA-Cmp)-NH₂ (PSM-0322);
HPA-Aib-Pcf-[Cys-Lys-Arg-Lys(DOTA-APAc)-Asn-Cys]-Tle-Thr-NH₂ (PSM-0324);
Ac-Thr-Aib-Phe-[Cys-Lys-Arg-Lys(DOTA)-Asn-Cys]-Tle-Thr-NH₂ (PSM-0326);
SaPr-Aib-Pcf-[Cys-Lys-Arg-Aib-Asn-Cys]-Tle-Thr-lys(DOTA)-NH₂ (PSM-0328);
Ac-Thr-Aib-Pcf-[Cys-Lys-Cit-Aib-Asn-Cys]-Tle-Thr-Cmp-lys(DOTA)-NH₂ (PSM-0329);
Ac-Thr-Ala-Phe-[Cys-Lys-Arg-Ala-Asn-Cys]-Leu-Thr-Asp-Ttds-lys(DOTA)-NH₂ (PSM-0330);
DOTA-Cmp-Thr-Aib-Pcf-[Smc-Lys-Arg-Aib-Asn-Cys]-Tle-NH₂
(alternative: DOTA-Cmp-Thr-Aib-Pcf-[Cys-Lys-Arg-Aib-Asn-Smc]-Tle-NH_{2) (PSM-0332);}
Ac-Thr-Aib-Phe-[Cys-Lys-Gln-Aib-Asn-Cys]-Tle-Thr-Asp-NH₂ (PSM-0334);
Ac-Thr-Ala-Phe-[Cys-Lys-Arg-Ala-Ala-Cys]-Nle-Thr-Asp-NH₂ (PSM-0335);
Ac-Thr-Aib-Pcf-[Cys-Lys-Arg-Aib-Asn-Cys]-Tle-Thr-NH₂ (PSM-0336);
Ac-Thr-Aib-Pcf-[Cys-Lys-Nle-Aib-Asn-Cys]-Tle-Thr-NH₂ (PSM-0338);
Ac-Thr-Aib-Phe-[Cys-Lys-Arg-Aib-Asn-Cys]-Tle-Thr-Cmp-lys(DOTAGA)-NH₂ (PSM-0339);
DOTA-Cmp-Thr-Aib-Pcf-[Cys-Lys-Nmr-Aib-Asn-Cys]-Tle-Thr-NH₂ (PSM-0340);
DOTA-Cmp-Thr-Aib-Pcf-[Cys-Lys-Lys(Ac)-Aib-Asn-Cys]-Tle-Thr-NH₂ (PSM-0341);
DOTA-Ttds-Thr-Ala-Phe-[Cys-Lys-Arg-Ala-Asn-Cys]-Tle-Thr-Asp-NH₂ (PSM-0342);
DOTA-Cmp-Thr-Aib-Pcf-[Cys-Lys-Arg(Me)-Aib-Asn-Cys]-Tle-NH₂ (PSM-0345);
DOTA-Cmp-Thr-Amd-Pcf-[Cys-Lys-Arg-Aib-Asn-Cys]-Tle-Thr-NH₂ (PSM-0346);
Ac-Aib-Pcf-[Cys-Lys-Arg-Aib-Asn-Cys]-Tle-Thr-Cmp-lys(DOTA)-NH₂ (PSM-0349);
Ac-Thr-Aib-Phe-[Cys-Lys-Arg-Aib-Asn-Cys]-leu-Thr-NH₂ (PSM-0350);
DOTA-Cmp-Thr-Aib-Pcf-[Cys-Lys-Arg(Me)-Aib-Asn-Cys]-Tle-Thr-NH₂ (PSM-0351);
DOTA-Bal-Aib-Pcf-[Cys-Lys-Arg-Aib-Asn-Cys]-Tle-Thr-NH₂ (PSM-0352);
Iva-Aib-Pcf-[Cys-Lys-Arg-Lys(DOTA-APAc)-Asn-Cys]-Tle-Thr-NH₂ (PSM-0353);
DOTA-Cmp-Thr-Aib-Mcf-[Cys-Lys(Me)-Arg(Me)-Aib-Asn-Cys]-Tle-NH₂ (PSM-0354);
DOTA-Cmp-Thr-Aib-Pcf-[Cys-Lys(Me)-RMe3-Aib-Asn-Cys]-Tle-Thr-NH₂ (PSM-0355);
DOTA-Cmp-Ile-Aib-Phe-[Cys-Lys-Arg-Aib-Asn-Cys]-Tle-Thr-NH₂ (PSM-0357);
DOTA-Cmp-Thr-Aib-Phe-[Cys-Lys-Arg-Aib-Asn-Cys]-Tle-NH₂ (PSM-0361);
Ac-Thr-Aib-Phe-[Cys-Lys-Glu-Ala-Asn-Cys]-Tle-Thr-Asp-NH₂ (PSM-0363);
DOTA-Cmp-Thr-Aib-Pcf-[Cys-Lys-Cit-Aib-Asn-Cys]-Tle-Thr-NH₂ (PSM-0365);
DOTA-Cmp-Thr-amd-Pcf-[Cys-Lys-Arg-Aib-Asn-Cys]-Tle-Thr-NH₂ (PSM-0366);
Ac-Thr-Aib-Pcf-[Cys-Lys-Arg-Aib-Asn-Cys]-Tle-NH₂ (PSM-0367);
DOTA-Ahx-Thr-Aib-Phe-[Cys-Lys-Arg-Aib-Asn-Cys]-Tle-Thr-NH₂ (PSM-0368);
DOTA-Cmp-Thr-Ams-Pcf-[Cys-Lys-Arg-Aib-Asn-Cys]-Tle-Thr-NH₂ (PSM-0369);
Ac-Thr-Aib-Pcf-[Cys-Lys-Arg-Lys(DOTA-O20c)-Asn-Cys]-Tle-Thr-NH₂ (PSM-0370);
nBuCAyl-Thr-Aib-Pcf-[Cys-Lys-Arg-Aib-Asn-Cys]-Tle-Thr-Cmp-lys(DOTA)-NH₂ (PSM-0371);
Ac-Thr-Aib-Pmf-[Cys-Lys-Arg-Aib-Asn-Cys]-Tle-Thr-NH₂ (PSM-0372);
Ac-Thr-Aib-Phe-[Cys-Lys-Arg-Aib-Asn-Cys]-Tle-Thr-Asp-NH₂ (PSM-0374);
Ac-Thr-Aib-Phe-[Cys-Lys-Arg-Deg-Asn-Cys]-Tle-Thr-NH₂ (PSM-0375);
Ac-Thr-Aib-Phe-[Cys-Lys-Arg-Aib-Asn-Cys]-Tle-Trp-NH₂ (PSM-0376);
DOTA-Cmp-Thr-Aib-Pcf-[Cys-Lys-Arg-Aib-Asn-Cys]-Val-Nmt-NH₂ (PSM-0377);
H-Cmp-Thr-Aib-Phe-[Cys-Lys-Arg-Aib-Asn-Cys]-Tle-Thr-Cmp-lys(DOTA)-NH₂ (PSM-0378);
Ac-Cmp-Thr-Aib-Pcf-[Cys-Lys-Arg-Aib-Asn-Cys]-Tle-en-DOTA (PSM-0379);
DOTA-Cmp-Thr-Aib-Pcf-[Cys-Lys(Me)-Arg(Me)-Aib-Asn-Cys]-Tle-NH₂ (PSM-0380);
DOTA-Pamb-Aib-Pcf-[Cys-Lys-Arg-Aib-Asn-Cys]-Tle-NH₂ (PSM-0381);
DOTA-Cmp-Thr-Aib-Pcf-[Cys-Lys-Arg-Ams-Asn-Cys]-Tle-Thr-NH₂ (PSM-0382);
Ac-Thr-Aib-Mcf-[Cys-Lys-Arg-Aib-Asn-Cys]-Tle-Thr-Asp-NH₂ (PSM-0383);
DOTA-Cmp-Thr-Aib-Pcf-[Cys-Lys-Arg-Aib-Asn-Cys]-Tle-en (PSM-0384);
Ac-Thr-Aib-Phe-[Cys-Lys-Gln-Lys(DOTA)-Asn-Cys]-Tle-Thr-NH₂ (PSM-0385);
Ac-Thr-Aib-Phe-[Cys-Lys-Arg-Aib-Asn-Cys]-Tle-Pro-NH₂ (PSM-0388);
Ac-Thr-Ala-Phe-[Cys-Lys-Arg-Ala-Asn-Cys]-Nle-Thr-asp-NH₂ (PSM-0389);
DOTA-Cmp-Thr-Aib-6Clw-[Cys-Lys(Me)-Arg(Me)-Aib-Asn-Cys]-Tle-NH₂ (PSM-0390);
Ac-Thr-Aib-Phe-[Cys-Lys-Arg-Aib-Asn-Cys]-Tle-Thr-Ape-DOTA (PSM-0391);
Ac-Thr-Aib-Phe-[Cys-Lys-Arg-4Tfp-Asn-Cys]-Tle-Thr-Asp-NH₂ (PSM-0392);
Ac-Thr-Aib-Pcf-[Cys-Lys-Arg-Aib-Asn-Cys]-Tle-en (PSM-0393);
Ac-Thr-Aib-Phe-[Cys-Lys-Arg-Aib-Asn-Cys]-Aib-Thr-NH₂ (PSM-0394);
DOTA-Cmp-Thr-Aib-Pff-[Cys-Lys(Me)-Arg(Me)-Aib-Asn-Cys]-Tle-NH₂ (PSM-0395);
DOTA-Cmp-Thr-Aib-Mtf-[Cys-Lys(Me)-Arg(Me)-Aib-Asn-Cys]-Tle-NH₂ (PSM-0396);
Ac-Thr-Aib-Phe-[Cys-Lys-Arg-Lys(DOTA)-Asn-Cys]-Tle-Thr-Asp-NH₂ (PSM-0397);
DOTA-Ttds-Thr-Aib-Phe-[Cys-Lys-Arg-Aib-Asn-Cys]-Tle-Thr-Asp-NH₂ (PSM-0398);
Hex-Thr-Aib-Pcf-[Cys-Lys-Arg-Aib-Asn-Cys]-Tle-Thr-lys(DOTA-Cmp)-NH₂ (PSM-0400);
Ac-Thr-Aib-Phe-[Cys-Lys-Arg-Lys(DOTA-O20c)-Asn-Cys]-Tle-Thr-NH₂ (PSM-0401);
Ac-Thr-Aib-Phe-[Cys-Lys-Arg-Aib-asn-Cys]-Tle-Thr-NH₂ (PSM-0402);
Ac-Thr-Aib-Phe-[Cys-Lys-Arg-Oic-Asn-Cys]-Tle-Thr-Asp-NH₂ (PSM-0403);
Ac-Aib-Pcf-[Cys-Lys-Arg-Aib-Asn-Cys]-Tle-Thr-lys(DOTA)-NH₂ (PSM-0404);
Hex-Thr-Ala-Phe-[Cys-Lys-Arg-Ala-Asn-Cys]-Leu-Thr-Asp-Ttds-lys(DOTA)-NH₂ (PSM-0405);
DOTA-Cmp-Thr-Ams-Pcf-[Cys-Lys-Arg-Ams-Asn-Cys]-Tle-Thr-NH₂ (PSM-0407);
Ac-Thr-Nmg-Phe-[Cys-Lys-Arg-Aib-Asn-Cys]-Tle-Thr-NH₂ (PSM-0408);
DOTA-Cmp-Thr-Aib-Opa-[Cys-Lys(Me)-Arg(Me)-Aib-Asn-Cys]-Tle-NH₂ (PSM-0409);
DOTA-Cmp-Thr-Aib-Pcf-[Cys-Lys(Me)-Arg-Aib-Asn-Cys]-Tle-Thr-NH₂ (PSM-0410);
Ac-Thr-Aib-Hfe-[Cys-Lys-Arg-Aib-Asn-Cys]-Tle-Thr-NH₂ (PSM-0411);
Ac-Thr-Aib-Mmf-[Cys-Lys-Arg-Aib-Asn-Cys]-Tle-Thr-NH₂ (PSM-0412);
DOTA-PPAc-Aib-Phe-[Cys-Lys-Arg-Aib-Asn-Cys]-Tle-Thr-NH₂ (PSM-0413);
Ac-Thr-Ala-Amf-[Cys-Lys-Arg-Aib-Asn-Cys]-Tle-Thr-NH₂ (PSM-0414);
Ac-Thr-Aib-Phe-[Cys-Lys-Arg-Lys(DOTA-Cmp)-Asn-Cys]-Tle-Thr-NH₂ (PSM-0415);
DOTA-Cmp-Thr-Aib-Pcf-[Cys-Lys-Arg-Glu-Asn-Cys]-Tle-Thr-NH₂ (PSM-0416);
Ac-Thr-Aib-Phe-[Cys-Lys-Arg-Lys(DOTA-Pamb)-Asn-Cys]-Tle-Thr-NH₂ (PSM-0419);
DOTA-Cmp-Thr-Aib-Pcf-[Smc-Lys(Me)-Arg(Me)-Aib-Asn-Cys]-Tle-NH₂
(alternative: DOTA-Cmp-Thr-Aib-Pcf-[Cys-Lys(Me)-Arg(Me)-Aib-Asn-Smc]-Tle-NH_{2) (PSM-0420);}
Ac-Thr-Aib-Pcf-[Cys-Lys-Cit-Aib-Asn-Cys]-leu-Thr-NH₂ (PSM-0421);
Ac-Thr-Aib-Phe-[Cys-Tap-Arg-Ala-Asn-Cys]-Tle-Thr-Asp-NH₂ (PSM-0422);
Ac-Thr-Aib-Phe-[Cys-Lys-Gln-Lys(DOTA-Ttds)-Asn-Cys]-Tle-Thr-NH₂ (PSM-0423);
Ac-Aib-Pcf-[Cys-Lys-Arg-Aib-Asn-Cys]-Tle-NH₂ (PSM-0424);
Hex-Thr-Aib-Phe-[Cys-Lys-Arg-Aib-Asn-Cys]-Tle-Thr-Asp-Ttds-lys(DOTA)-NH₂ (PSM-0425);
Ac-Thr-Aib-Phe-[Cys-Lys-Arg-Aib-Asn-Cys]-Tle-Bal-NH₂ (PSM-0426);
nBuCAyl-Thr-Aib-Pcf-[Cys-Lys-Opy-Aib-Asn-Cys]-Tle-Thr-Cmp-lys(DOTA)-NH₂ (PSM-0427);
Ac-Thr-Aib-Phe-[Cys-Lys-Arg-Aib-Asn-Cys]-Tle-Thr-Cmp-lys(DOTA)-NH₂ (PSM-0428);
DOTA-Cmp-Thr-Aib-Mcf-[Cys-Lys-Cit-Aib-Asn-Cys]-Tle-Thr-NH₂ (PSM-0431);
DOTA-Pamb-Aib-Pcf-[Cys-Lys-Arg-Aib-Asn-Cys]-Tle-Bal-NH₂ (PSM-0432);
nBuCAyl-Thr-Aib-Phe-[Cys-Lys-Cit-Aib-Asn-Cys]-Tle-Thr-Cmp-lys(DOTA)-NH₂ (PSM-0434);
Ac-Thr-Aib-Phe-[Cys-Gln-Arg-Ala-Asn-Cys]-Tle-Thr-Asp-NH₂ (PSM-0435);
DOTA-Bal-Aib-Phe-[Cys-Lys-Arg-Aib-Asn-Cys]-Tle-Thr-NH₂ (PSM-0437);
Ac-Thr-Aib-Phe-[Cys-Aph-Arg-Ala-Asn-Cys]-Tle-Thr-Asp-NH₂ (PSM-0439);
Ac-Thr-Aib-Cys(Bzl)-[Cys-Lys-Arg-Aib-Asn-Cys]-Tle-Thr-NH₂ (PSM-0441);
Ac-Aib-Phe-[Cys-Lys-Arg-Aib-Asn-Cys]-Tle-Thr-Asp-NH₂ (PSM-0442);
Ac-Aib-Pcf-[Cys-Lys-Arg-Aib-Asn-Cys]-NH₂ (PSM-0443);
Iva-Aib-Pcf-[Cys-Lys-Arg-Aib-Asn-Cys]-Tle-Thr-lys(DOTA)-NH₂ (PSM-0444);
DOTA-Cmp-Thr-Aib-Pcf-[Cys-Lys-Arg-Aib-Asn-Cys]-Tle-Thr-NH₂ (PSM-0445);
DOTA-Pab-Thr-Aib-Phe-[Cys-Lys-Arg-Aib-Asn-Cys]-Tle-Thr-NH₂ (PSM-0448);
DOTA-Cmp-Thr-Aib-Phe-[Cys-Lys-Arg-Aib-Asn-Cys]-Tle-Bal-NH₂ (PSM-0449);
Iva-Aib-Phe-[Cys-Lys-Arg-Aib-Asn-Cys]-Tle-Thr-NH₂ (PSM-0450);
Ac-Thr-Aib-Phe-[Cys-Nmk-Arg-Aib-Asn-Cys]-Tle-Thr-NH₂ (PSM-0451);
nBuCAyl-Thr-Aib-Pcf-[Cys-Lys-Arg-Aib-Asn-Cys]-Tle-Thr-lys(DOTA-Cmp)-NH₂ (PSM-0452);
Ac-Thr-Aib-Phe-[Cys-Lys-Arg-Aib-Asn-Cys]-Tle-Leu-NH₂ (PSM-0453);
DOTA-Cmp-Thr-Aib-Ppa-[Cys-Lys-Cit-Aib-Asn-Cys]-Tle-Thr-NH₂ (PSM-0454);
DOTA-Cmp-Nmt-Ala-Pcf-[Cys-Lys-Arg-Aib-Asn-Cys]-Tle-Thr-NH₂ (PSM-0455);
DOTA-Cmp-Thr-Aib-Pcf-[Cys-Lys-Arg-Aib-Asn-Cys]-Tle-en(Me) (PSM-0456);
DOTA-Pamb-Aib-Phe-[Cys-Lys-Arg-Aib-Asn-Cys]-Tle-NH₂ (PSM-0458);
DOTA-Cmp-Thr-Aib-Mnf-[Cys-Lys(Me)-Arg(Me)-Aib-Asn-Cys]-Tle-NH₂ (PSM-0459);
Ac-Thr-Ala-Phe-[Cys-Lys-Arg-Pro-Asn-Cys]-Nle-Thr-Asp-NH₂ (PSM-0460);
Hib-Pcf-[Cys-Lys-Arg-Aib-Asn-Cys]-Tle-Thr-NH₂ (PSM-0461);
Hex-Thr-Aib-Phe-[Cys-Lys-Arg-Ala-Asn-Cys]-Tle-Thr-Ttds-lys(DOTA)-NH₂ (PSM-0462);
Ac-Thr-Aib-Phe-[Cys-Lys-Arg-Lys(DOTA-APAc)-Asn-Cys]-Tle-Thr-NH₂ (PSM-0464);
Ac-Thr-Aib-Phe-[Cys-Lys-Arg-Lys(DOTA-Ttds)-Asn-Cys]-Tle-Thr-NH₂ (PSM-0465);
DOTA-Cmp-Thr-Aib-Pcf-[Cys-Lys(Ac)-Arg-Aib-Asn-Cys]-Tle-Thr-NH₂ (PSM-0466);
DOTA-Cmp-Thr-Aib-Pcf-[Cys-Lys-Arg-Aib-Asn-Cys]-Tle-Bal-NH₂ (PSM-0467);
Ac-Ser-Ala-Phe-[Cys-Lys-Arg-Ala-Asn-Cys]-Nle-Thr-Asp-NH₂ (PSM-0469);
nBuCAyl-Thr-Aib-Mcf-[Cys-Lys-Arg-Aib-Asn-Cys]-Tle-Thr-Cmp-lys(DOTA)-NH₂ (PSM-0470);
Ac-Thr-Aib-Phe-[Cys-Lys-Arg-Aib-Asn-Cys]-Tle-Thr-Pab-lys(DOTA)-NH₂ (PSM-0471);
Ac-Aib-Pcf-[Cys-Lys(Me)-Arg-Aib-Asn-Cys]-Tle-en-DOTA (PSM-0472);
Ac-Thr-Ala-Phe-[Cys-Lys-Arg-Ala-Asn-Cys]-Nle-Ala-Asp-NH₂ (PSM-0476);
Ac-Thr-Aib-Phe-[Cys-Lys-Arg-Aib-Asn-Cys]-Tle-Gly-NH₂ (PSM-0477);
Ac-Thr-Aib-Phe-[Cys-Lys-Arg-Aib-Asp-Cys]-Tle-Thr-NH₂ (PSM-0478);
Ac-Thr-Aib-Phe-[Cys-Lys-Arg-Aib-Gly-Cys]-Tle-Thr-NH₂ (PSM-0479);
DOTA-Ttds-Thr-Ala-Phe-[Cys-Lys-Arg-Ala-Asn-Cys]-Leu-NH₂ (PSM-0480);
Ac-Aib-Pcf-[Cys-Lys(Me)-Arg(Me)-Aib-Asn-Cys]-Tle-en-DOTA (PSM-0481);
Ac-Thr-Aib-Pcf-[Cys-Lys-Arg-Aib-Asn-Cys]-Tle-Thr-Cmp-lys(DOTA)-NH₂ (PSM-0482);
DOTA-Cmp-Thr-Aib-Pcf-[Cys-KMe3-Arg(Me)-Aib-Asn-Cys]-Tle-Thr-NH₂ (PSM-0483);
nBuCAyl-Thr-Aib-Phe-[Cys-Lys-Arg-Aib-Asn-Cys]-Tle-Thr-Cmp-lys(DOTA)-NH₂ (PSM-0484);
DOTA-Cmp-Thr-Aib-Pcf-[Cys-Hgn-Arg-Aib-Asn-Cys]-Tle-Thr-NH₂ (PSM-0485);
Ac-Thr-Aib-Mcf-[Cys-Lys-Arg-Aib-Asn-Cys]-Tle-Thr-Cmp-lys(DOTA)-NH₂ (PSM-0486);
Ac-Thr-Aib-Tyr-[Cys-Lys-Arg-Aib-Asn-Cys]-Tle-Thr-Asp-NH₂ (PSM-0488);
DOTA-Cmp-Aib-Pcf-[Cys-Lys-Gln(Gu)-Aib-Asn-Cys]-Tle-Thr-NH₂ (PSM-0489);
DOTA-Cmp-Thr-Aib-Pcf-[Cys-Lys(Bio)-Arg-Aib-Asn-Cys]-Tle-Thr-NH₂ (PSM-0490);
Ac-Ala-Ala-Phe-[Cys-Lys-Arg-Ala-Asn-Cys]-Nle-Thr-Asp-NH₂ (PSM-0491);
Ac-Thr-Aib-Pcf-[Cys-Lys-Arg-Aib-Asn-Cys]-Tle-Thr-OH (PSM-0493);
Ac-Aib-Pcf-[Cys-Lys-Gln(Gu)-Aib-Asn-Cys]-Tle-Thr-Cmp-lys(DOTA)-NH₂ (PSM-0494);
Crown-Cmp-Thr-Aib-Pcf-[Cys-Lys(Me)-Arg(Me)-Aib-Asn-Cys]-Tle-Thr-NH₂ (PSM-0495);
Ac-Thr-Aib-Pcf-[Cys-Lys-Cit-Aib-Glu-Cys]-Tle-Thr-NH₂ (PSM-0496);
DOTA-Cmp-Thr-Aib-Pcf-[Cys-Lys-Arg-Aib-Asn-Cys]-Nml-Thr-NH₂ (PSM-0497);
H-Met-Thr-Ala-Phe-[Cys-Lys-Arg-Ala-Asn-Cys]-Leu-Thr-Asp-Gly-Ser-NH₂ (PSM-0498);
DOTA-Cmp-Thr-Aib-Pcf-[Cys-Lys(Me)-Egd-Aib-Asn-Cys]-Tle-Thr-NH₂ (PSM-0499);
nBuCAyl-Thr-Aib-Pcf-[Cys-Lys-Cit-Aib-Asn-Cys]-Tle-Thr-lys(DOTA-Cmp)-NH₂ (PSM-0500);
Ac-Thr-Aib-Phe-[Cys-Lys-Arg-Ala-Asn-Cys]-Nle-Thr-Asp-NH₂ (PSM-0501);
Hex-Thr-Aib-Phe-[Cys-Lys-Arg-Ala-Asn-Cys]-Tle-Thr-Asp-Ttds-lys(DOTA)-NH₂ (PSM-0502);
DOTA-Cmp-Thr-Aib-Mmf-[Cys-Lys(Me)-Arg(Me)-Aib-Asn-Cys]-Tle-NH₂ (PSM-0503);
DOTA-Ttds-Thr-Ala-Phe-[Cys-Lys-Arg-Ala-Asn-Cys]-Nle-Thr-Asp-NH₂ (PSM-0504);
DOTA-O2Oc-Thr-Aib-Phe-[Cys-Lys-Arg-Aib-Asn-Cys]-Tle-Thr-NH₂ (PSM-0505);
Ac-Aib-Phe-[Cys-Lys-Arg-Ala-Asn-Cys]-Tle-Thr-Asp-NH₂ (PSM-0506);
DOTA-Cmp-Aib-Phe-[Cys-Lys-Arg-Aib-Asn-Cys]-Tle-Thr-NH₂ (PSM-0507);
Ac-Thr-Pam-Phe-[Cys-Lys-Arg-Aib-Asn-Cys]-Tle-Thr-NH₂ (PSM-0508);
HPA-Aib-Pcf-[Cys-Lys-Arg-Aib-Asn-Cys]-Tle-Thr-Cmp-lys(DOTA)-NH₂ (PSM-0509);
Ac-Thr-Aib-Phe-[Cys-Lys-Arg-Aib-Asn-Cys]-Tle-NHBu (PSM-0510);
DOTA-Cmp-Thr-Aib-Pcf-[Cys-Orn-Arg-Aib-Asn-Cys]-Tle-Thr-NH₂ (PSM-0511);
DOTA-Cmp-Thr-Aib-Opa-[Cys-Lys-Cit-Aib-Asn-Cys]-Tle-Thr-NH₂ (PSM-0512);
DOTA-Ahx-Aib-Phe-[Cys-Lys-Arg-Aib-Asn-Cys]-Tle-Thr-NH₂ (PSM-0513);
Ac-Thr-Aib-Pcf-[Cys-Lys-Arg-Aib-Asn-Cys]-Tle-Bal-NH₂ (PSM-0514);
Macropa-Cmp-Thr-Aib-Pcf-[Cys-Lys(Me)-Arg(Me)-Aib-Asn-Cys]-Tle-Thr-NH₂ (PSM-0515);
Ac-Aib-Pcf-[Cys-Lys-Arg-Aib-Asn-Cys]-Tle-en-DOTA (PSM-0516);
Ac-Thr-Aib-2Ni-[Cys-Lys-Arg-Aib-Asn-Cys]-Tle-Thr-Asp-NH₂ (PSM-0517);
HYDAc-Aib-Phe-[Cys-Lys-Arg-Aib-Asn-Cys]-Tle-Thr-NH₂ (PSM-0518);
DOTA-Ttds-Thr-Aib-Phe-[Cys-Lys-Gln-Aib-Asn-Cys]-Tle-Thr-NH₂ (PSM-0521);
Ac-Thr-Aib-Phe-[Cys-Lys-Arg-Aib-Asn-Cys]-Tle-Thr-Pamb-lys(DOTA)-NH₂ (PSM-0522);
Ac-Thr-Aib-Phe-[Cys-Lys-Arg-Aib-Asn-Cys]-Tle-Thr-O2Oc-lys(DOTA)-NH₂ (PSM-0529);
HPA-Aib-Phe-[Cys-Lys-Arg-Aib-Asn-Cys]-Tle-Thr-NH₂ (PSM-0530);
DOTA-Cmp-Thr-Aib-Pcf-[Cys-Lys-Hgn-Aib-Asn-Cys]-Tle-Thr-NH₂ (PSM-0531);
Ac-Thr-Aib-Phe-[Cys-Lys-Arg-Dtc-Asn-Cys]-Tle-Thr-Asp-NH₂ (PSM-0532);
Ac-Thr-Ala-Phe-[Cys-Lys-Arg-Ala-Asn-Cys]-Nle-Thr-Asp-Ttds-lys(DOTA)-NH₂ (PSM-0533);
DOTA-Thr-Aib-Pcf-[Cys-Lys-Arg-Aib-Asn-Cys]-Tle-NH₂ (PSM-0534);
Ac-Thr-Aib-Phe-[Cys-lys-Arg-Ala-Asn-Cys]-Tle-Thr-Asp-NH₂ (PSM-0535);
DOTA-Cmp-Tle-Aib-Pcf-[Cys-Lys(Me)-Arg(Me)-Aib-Asn-Cys]-Tle-Thr-NH₂ (PSM-0538);
DOTA-Thr-Aib-Phe-[Cys-Lys-Arg-Aib-Asn-Cys]-Tle-Bal-NH₂ (PSM-0539);
Ac-Thr-Aib-Phe-[Cys-Lys-Arg-Aib-Asn-Cys]-Tle-Gln-NH₂ (PSM-0540);
Ac-Thr-Aib-Phe-[Cys-Lys-Arg-Aib-Phe-Cys]-Tle-Thr-NH₂ (PSM-0541);
Ac-Thr-Aib-Phe-[Cys-Lys-Arg-Lys(DOTA-Ttds)-Asn-Cys]-Tle-Thr-Asp-NH₂ (PSM-0542);
DOTA-APAc-Thr-Aib-Phe-[Cys-Lys-Arg-Aib-Asn-Cys]-Tle-Thr-NH₂ (PSM-0543);
Ac-Thr-Aib-Phe-[Cys-Lys-Arg-Aib-Leu-Cys]-Tle-Thr-NH₂ (PSM-0545);
DOTA-Cmp-Thr-Aib-Pcf-[Cys-Lys-Urr-Aib-Asn-Cys]-Tle-Thr-NH₂ (PSM-0546);
Ac-Thr-Pro-Phe-[Cys-Lys-Arg-Ala-Asn-Cys]-Nle-Thr-Asp-NH₂ (PSM-0547);
DOTA-Cmp-Thr-Aib-Pcf-[Cys-Nmk-Arg-Aib-Asn-Cys]-Tle-Thr-NH₂ (PSM-0548);
DOTA-Cmp-Thr-Aib-Ptf-[Cys-Lys(Me)-Arg(Me)-Aib-Asn-Cys]-Tle-NH₂ (PSM-0549);
Ac-Thr-Aib-Ptf-[Cys-Lys-Arg-Aib-Asn-Cys]-Tle-Thr-NH₂ (PSM-0550);
Ac-Thr-Aib-Mff-[Cys-Lys-Arg-Aib-Asn-Cys]-Tle-Thr-NH₂ (PSM-0551);
Ac-Thr-Aib-Phe-[Cys-Lys-Arg-Ala-Asn-Cys]-Tle-Thr-NH₂ (PSM-0552);
DOTA-Cmp-Thr-Aib-Mff-[Cys-Lys(Me)-Arg(Me)-Aib-Asn-Cys]-Tle-NH₂ (PSM-0553);
Ac-Thr-Aib-Pcf-[Cys-Lys-Arg-Aib-Asn-Cys]-Tle-Throl-OH (PSM-0554);
Ac-Thr-Aib-Mnf-[Cys-Lys-Arg-Aib-Asn-Cys]-Tle-Thr-NH₂ (PSM-0555);
Ac-Thr-Aib-Phe-[Cys-Lys-Arg-Aib-Asn-Cys]-Tle-Phe-NH₂ (PSM-0556);
Ac-Thr-Aib-Ocf-[Cys-Lys-Arg-Aib-Asn-Cys]-Tle-Thr-Asp-NH₂ (PSM-0558);
Ac-Aib-Pcf-[Cys-Lys-Arg-Aib-Asn-Cys]-Tle-Thr-NH₂ (PSM-0559);
Ac-Thr-Aib-Phe-[Cys-Lys-Arg-Aib-Asn-Cys]-Npg-NH₂ (PSM-0560);
Ac-Thr-Ala-Phe-[Cys-Lys-Arg-Ala-Asn-Cys]-Nle-Thr-Ala-NH₂ (PSM-0562);
Ac-Thr-Aib-Phe-[Cys-Lys-Arg-Aib-Glu-Cys]-Tle-Thr-NH₂ (PSM-0563);
Ac-Thr-Aib-Phe-[Cys-Lys-Arg-Ala-Asn-Cys]-Tle-Thr-Asp-NH₂ (PSM-0565);
Ac-Thr-Aib-Phe-[Smc-Lys-Arg-Ala-Asn-Cys]-Tle-Thr-Asp-NH₂
(alternative: Ac-Thr-Aib-Phe-[Cys-Lys-Arg-Ala-Asn-Smc]-Tle-Thr-Asp-NH_{2) (PSM-0567);}
Hex-Thr-Ala-Phe-[Cys-Lys-Arg-Ala-Asn-Cys]-Tle-Thr-Asp-Ttds-lys(DOTA)-NH₂ (PSM-0568);
Ac-Thr-Aib-Pcf-[Cys-Lys-Arg(Ac)-Aib-Asn-Cys]-Tle-Thr-NH₂ (PSM-0569);
Ac-Thr-Aib-Pcf-[Cys-Nle-Arg(Ac)-Aib-Asn-Cys]-Tle-Thr-NH₂ (PSM-0570);
nBuCAyl-Thr-Aib-Mcf-[Cys-Lys-Opy-Aib-Asn-Cys]-Tle-Thr-Cmp-lys(DOTA)-NH₂ (PSM-0571);
DOTA-Thr-Aib-Pcf-[Cys-Lys-Arg-Aib-Asn-Cys]-Tle-Thr-NH₂ (PSM-0573);
DOTA-Pamb-Aib-Phe-[Cys-Lys-Arg-Aib-Asn-Cys]-Tle-Bal-NH₂ (PSM-0574);
Bio-Ttds-Ttds-Thr-Aib-Pcf-[Cys-Lys-Arg-Aib-Asn-Cys]-Tle-Thr-NH₂ (PSM-0575);
Hex-Thr-Aib-Phe-[Cys-Lys-Arg-Aib-Asn-Cys]-Tle-Thr-Ttds-lys(DOTA)-NH₂ (PSM-0576);
HO-Succinyl-Aib-Phe-[Cys-Lys-Arg-Aib-Asn-Cys]-Tle-Thr-NH₂ (PSM-0577);
Ac-Thr-Ala-Phe-[Cys-Lys-Arg-Aib-Asn-Cys]-Nle-Thr-Asp-NH₂ (PSM-0578);
DOTA-Cmp-Thr-Aib-Pcf-[Cys-Lys-Arg(EtCAyl)-Aib-Asn-Cys]-Tle-Thr-NH₂ (PSM-0579);
DOTA-Thr-Aib-Phe-[Cys-Lys-Arg-Aib-Asn-Cys]-Tle-Thr-NH₂ (PSM-0580);
Ac-Thr-Aib-Phe-[Cys-Lys-Arg-Lys(DOTA-Ahx)-Asn-Cys]-Tle-Thr-NH₂ (PSM-0582);
DOTA-Cmp-Thr-Aib-Mcf-[Cys-Lys-Arg-Aib-Asn-Cys]-Tle-Thr-NH₂ (PSM-0583);
Ac-Thr-Aib-Pcf-[Cys-Lys-Cit-Aib-Leu-Cys]-Tle-Thr-NH₂ (PSM-0584);
DOTA-Cmp-Thr-Aib-Pcf-[Cys-Lys-Arg-Ala-Nmn-Cys]-Tle-Thr-NH₂ (PSM-0585);
Ac-Thr-Aib-Trp-[Cys-Lys-Arg-Aib-Asn-Cys]-Tle-Thr-Asp-NH₂ (PSM-0587);
Ac-Thr-Aib-Phe-[Cys-Lys-Arg-Aib-Trp-Cys]-Tle-Thr-NH₂ (PSM-0589);
DOTA-Cmp-Thr-Aib-Pcf-[Cys-Lys(Me)-RMe2a-Aib-Asn-Cys]-Tle-Thr-NH₂ (PSM-0590):
Macropa-Cmp-Thr-Aib-Pcf-[Cys-Lys(Me)-Arg(Me)-Aib-Asn-Cys]-Tle-Thr-NH2 (PSM-0591);
DOTA-Cmp-Thr-Aib-Pcf-[Cys-Lys(Me)-RMe1-Aib-Asn-Cys]-Tle-Thr-NH2 (PSM-0592);
DOTAM-Cmp-Thr-Aib-Pcf-[Cys-Lys(Me)-Arg(Me)-Aib-Asn-Cys]-Tle-Thr-NH2 (PSM-0593);
DOTA-Cmp-Thr-Aib-Pcf-[Cys-Lys(Me)-Arg(Me)-Aib-Asn-Cys]-OH (PSM-0594);
LSC-Cmp-Thr-Aib-Pcf-[Cys-Lys(Me)-Arg(Me)-Aib-Asn-Cys]-Tle-Thr-NH2 (PSM-0601);
DOTA-Cmp-Thr-Aib-Phe-[Cys-Lys(Me)-Arg(Me)-Aib-Asn-Cys]-Tle-en-H (PSM-0605)
and
DOTA-Cmp-Thr-Aib-Pcf-[Cys-Lys(Me)-Arg(Me)-Aib-Asn-Cys]-Tle-Bal-NH2(PSM-0606).

58. The compound of claim 1, or pharmaceutically acceptable salt, solvate or hydrate thereof, wherein the compound comprises a diagnostically active nuclide or a therapeutically active nuclide.

59. The compound of claim 58, or pharmaceutically acceptable salt, solvate or hydrate thereof, wherein the compound comprises a diagnostically active nuclide; preferably diagnostically active nuclide is a diagnostically active radionuclide, more preferably the diagnostically active radionuclide is selected from the group consisting of ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I.

60. The compound of claim 1, or pharmaceutically acceptable salt, solvate or hydrate thereof, wherein the compound comprises a therapeutically active nuclide; preferably the therapeutically active nuclide is a therapeutically active radionuclide; more preferably the therapeutically active radionuclide is selected from the group consisting of ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, and ²¹¹At.

61. The compound or pharmaceutically acceptable salt, solvate or hydrate thereof of claim 1, for use in a method for the diagnosis of a disease.

62. The compound or pharmaceutically acceptable salt, solvate or hydrate thereof of claim 1, for use in a method for the treatment of a disease.

63. The compound or pharmaceutically acceptable salt, solvate or hydrate thereof for use of claim 61, wherein the disease involves cells showing upregulated expression of prostate specific membrane antigen (PSMA), preferably diseased tissue containing cells showing upregulated expression of PSMA.

64. The compound or pharmaceutically acceptable salt, solvate or hydrate thereof for use of claim 61, wherein the disease is a neoplasm, preferably a cancer or tumor; more preferably the tumor is selected from the group comprising an advanced tumor, a metastatic tumor, and a primary tumor; and most preferably the tumor is selected
a) from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, a carcinoma, a squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), and combinations thereof, or
b) from the group comprising: prostate cancer (e.g., metastatic castration resistant prostate cancer), renal cancer (e.g., clear cell carcinoma), head cancer, neck cancer, head and neck cancer, lung cancer (e.g., non-small cell lung cancer), salivary gland cancer, breast cancer, colorectal cancer, esophageal cancer, stomach cancer, liver cancer (e.g., hepatocellular cancer), thyroid cancer, glioblastoma, glioma, gall bladder cancer, laryngeal cancer, leukemia/lymphoma, uterine cancer, skin cancer (e.g., melanoma), endocrine cancer, sarcoma, urinary cancer, pancreatic cancer, gastrointestinal cancer, ovarian cancer, cervical cancer, endometrial cancer, fallopian tube cancer, primary peritoneal cancer, hematological cancer (e.g., diffuse large B cell lymphoma, Hodgkin's lymphoma, Non-Hodgkin's lymphoma, follicular lymphoma, acute myeloid leukemia, or multiple myeloma), cancer of unknown primary, adenomas, and tumor neovasculature.

65. The compound or pharmaceutically acceptable salt, solvate or hydrate thereof for use of claim 61, wherein the method for the diagnosis is an imaging method.

66. The compound or pharmaceutically acceptable salt, solvate or hydrate thereof of claim 1, for use in a method for the identification of a subject, wherein the subject is likely to respond or likely not to respond to a treatment of a disease, wherein the method for the identification of a subject comprises carrying out a method of diagnosis using the compound or pharmaceutically acceptable salt, solvate or hydrate thereof.

67. The compound or pharmaceutically acceptable salt, solvate or hydrate thereof of claim 1, for use in a method for the selection of a subject from a group of subjects, wherein the subject is likely to respond or likely not to respond to a treatment of a disease, wherein the method for the selection of a subject from a group of subjects comprises carrying out a method of diagnosis using the compound or pharmaceutically acceptable salt, solvate or hydrate thereof.

68. The compound or pharmaceutically acceptable salt, solvate or hydrate thereof of claim 1, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosis using the compound or pharmaceutically acceptable salt, solvate or hydrate thereof.

69. The compound or pharmaceutically acceptable salt, solvate or hydrate thereof for use of claim 66, wherein the disease is a disease involving the prostate specific membrane antigen (PSMA) protein, or wherein the disease involves cells showing upregulated expression of prostate specific membrane antigen (PSMA); preferably the tumor is selected from the group comprising a prostate tumor, a metastasized prostate tumor, a lung tumor, a renal tumor, a glioblastoma, a pancreatic tumor, a bladder tumor, a sarcoma, a melanoma, a breast tumor, a colon tumor, a pheochromocytoma, an esophageal tumor, a stomach tumor, a carcinoma, a squamous carcinoma (e.g., cervical canal, eyelid, tunica conjunctiva, vagina, lung, oral cavity, skin, urinary bladder, tongue, larynx, and gullet), and an adenocarcinoma (e.g., prostate, small intestine, endometrium, cervical canal, large intestine, lung, pancreas, gullet, rectum, uterus, stomach, mammary gland, and ovary), and combinations thereof.

70. The compound or pharmaceutically acceptable salt, solvate or hydrate thereof of claim 1, for use in a method for delivering a diagnostically active radionuclide or a therapeutically active radionuclide to prostate specific membrane antigen (PSMA); preferably the diagnostically active radionuclide is selected from the group consisting of ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I, preferably ¹⁸F, ⁴³Sc, ⁴⁴Sc, ⁶⁴Cu, ⁶⁷Ga, ⁶⁸Ga, ⁸⁶Y, ⁸⁹Zr, ^{99m}Tc, ¹¹¹In, ¹⁵²Tb, ¹⁵⁵Tb, and ²⁰³Pb, and more preferably ¹⁸F, ⁶⁴Cu, ⁶⁸Ga, and ¹¹¹In, and the therapeutically active radionuclide is selected from the group consisting of ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ac, ²²⁶Th, ²²⁷Th, ¹³¹I, ²¹¹At, preferably ⁴⁷Sc, ⁶⁷Cu, ⁹⁰Y, ¹⁶¹Tb, ¹⁷⁷Lu, ¹⁸⁸Re, ²¹²Pb, ²¹²Bi, ²¹³Bi, ²²⁵Ac, 227Th, and more preferably ⁹⁰Y, ¹⁶¹Tb, ¹⁷⁷Lu, ²¹²Pb, ²²⁵Ac, and ²²⁷Th.

71. The compound or pharmaceutically acceptable salt, solvate or hydrate thereof for use of claim 70, wherein the prostate specific membrane antigen (PSMA) is expressed by a cell, preferably a prostate cell, a metastasized prostate cell, a lung cell, a renal cell, a pancreatic cell, a bladder cell, a breast cell, a colon cell, a germ cell, an esophageal cell, a stomach cell, an endothelial cell and combinations thereof each showing upregulated expression of PSMA.

72. A composition, preferably a pharmaceutical composition, wherein the composition comprises a compound or pharmaceutically acceptable salt, solvate or hydrate thereof according to claim 1, and a pharmaceutically acceptable excipient.

73. A method for the diagnosis of a disease in a subject, wherein the method comprises administering to the subject a diagnostically effective amount of a compound or pharmaceutically acceptable salt, solvate or hydrate thereof according to claim 1; preferably the compound or pharmaceutically acceptable salt, solvate or hydrate thereof comprises a diagnostically active nuclide, whereby the nuclide is preferably a diagnostically active radionuclide.

74. A method for the treatment of a disease in a subject, wherein the method comprises administering to the subject a therapeutically effective amount of a compound or pharmaceutically acceptable salt, solvate or hydrate thereof according to claim 1; preferably the compound or pharmaceutically acceptable salt, solvate or hydrate thereof comprises a therapeutically active nuclide, whereby the nuclide is preferably a therapeutically active radionuclide.

75. The method of claim 73, wherein the disease is a disease involving the prostate specific membrane antigen (PSMA) protein; or the disease involves cells showing upregulated expression of prostate specific membrane antigen (PSMA), preferably diseased tissue containing cells showing upregulated expression of PSMA.

76. A kit comprising a compound or pharmaceutically acceptable salt, solvate or hydrate thereof according to claim 1, one or more optional excipient(s) and optionally one or more device(s), whereby the device(s) is/are selected from the group comprising a labeling device, a purification device, a handling device, a radioprotection device, an analytical device or an administration device.

77. The kit of claim 76 for use in any method as defined in the specification.
